(19) United States
(12) Patent Application Publication
(10) Pub. No.: US 2023/0116689 A1
(43) Pub. Date: Apr. 13, 2023

(54) METHODS AND BIOLOGICAL SYSTEMS FOR DISCOVERING AND OPTIMIZING LASSO PEPTIDES

(71) Applicant: Lassogen, Inc., San Diego, CA (US)

(72) Inventors: Mark J. Burk, San Diego, CA (US); I-Hsiung Brandon Chen, La Mesa, CA (US)

(21) Appl. No.: 17/906,102

(22) PCT Filed: Mar. 18, 2021
(86) PCT No.: PCT/US2021/023000

§ 371 Date: Sep. 12, 2022

Related U.S. Application Data
(60) Provisional application No. 62/992,105, filed on Mar. 19, 2020.

(51) Int. Cl. C12N 15/10; G01N 33/68; C07K 14/005

(52) U.S. Cl. CPC C12N 15/1037; C12N 15/1058; G01N 33/6845; C07K 14/005; C07K 2319/02; C07K 2319/034; C07K 2319/50; C12N 2795/10122; C12N 2795/10141; C12N 2795/14122; C12N 2795/14141

(57) ABSTRACT

Provided herein are lasso peptides libraries, and particularly phage display libraries of lasso peptides. Also provided herein are related methods and systems for producing the libraries and for screening the libraries to identify candidate lasso peptides having desirable properties.

This application claims the benefit of priority to U.S. Provisional Patent Application No. 62/992,105 filed Mar. 19, 2020, the disclosure of which is incorporated by reference herein in its entirety.

The instant application contains a Sequence Listing which has been submitted electronically in ASCII format and is hereby incorporated by reference in its entirety. Said ASCII copy, created on Mar. 18, 2021, is named 14619-008-228_Sequence Listing.txt and is 1,710,453 bytes in size.

### 1. FIELD

Provided herein are biological systems and related methods for discovering and optimizing lasso peptides.

### 2. BACKGROUND

Peptides serve as useful tools and leads for drug development since they often combine high affinity and specificity for their target receptor with low toxicity. However, their clinical use as efficacious drugs has been limited due to undesirable physicochemical and pharmacokinetic properties, including poor solubility and cell permeability, low bioavailability, and instability due to rapid proteolytic degradation under physiological conditions.

Ribosomally assembled natural peptides having a knotted topology may be used as molecular scaffold for drug design. For example, ribosomally assembled natural peptides sharing the cyclic cystine knot (CCK) motif as exemplified by the cyclotides and conotoxins, recently have been introduced as stable molecular frameworks for potential therapeutic applications (Weidmann, J.; Craik, D. J., J. Experimental Bot., 2016, 67, 4801-4812; Burman, R., et al., J. Nat. Prod. 2014, 77, 724-736; Reinwarth, M., et al., Molecules, 2012, 17, 12533-12552; Lewis, R. J., et al., Pharmacol. Rev., 2012, 64, 259-298). But these knotted peptides require the formation of three disulfide bonds to hold them into a defined conformation. As the biosynthetic machinery of plant-derived cyclotides and animal-derived conotoxins is not well understood, these knotted peptide scaffolds are not readily accessible by genetic manipulation and heterologous production in cells and discovery relies on traditional extraction and fractionation methods that are slow and costly. Moreover, their production relies either on solid phase peptide synthesis (SPPS) or on expressed protein ligation (EPL) methods to generate the circular peptide backbone, followed by oxidative folding to form the correct three disulfide bonds required for the knotted structure (Craik, D. J., et al., Cell Mol. Life Sci. 2010, 67, 9-16; Berrade, L. & Camarero, J. A. Cell Mol. Life Sci., 2009, 66, 3909-22).

There exists a need for new classes of peptide-based diagnostic and therapeutic compounds with readily available methods for their discovery, genetic manipulation and evolution, cost-effective production, and high-throughput screening. The present disclosure provided herein meet these needs.

### 3. SUMMARY

Provided herein are lasso peptides and related molecules, libraries and compositions. Also provided herein are methods for optimizing and screening lasso peptide libraries for candidates having desirable properties.

In one aspect, provided herein are fusion proteins comprising a bacteriophage coat protein fused to a lasso peptide component. In some embodiments the bacteriophage coat protein comprises p3, p6, p7, p8 or p9 of filamentous phages, small outer capsid (SOC) protein or highly antigenic outer capsid (HOC) protein of a T4 phage, pX of a T7 phage, pD or pV of a (lambda) phage or a functional variant thereof. In some embodiments, the functional variant is selected from a truncation, deletion, insertion, mutation, conjugation, domain-shuffling or domain-swapping.

In some embodiments, the lasso peptide component is a lasso precursor peptide, a lasso core peptide, a lasso peptide or a functional fragment of lasso peptide. In some embodiments, the lasso precursor peptide comprises a sequence of any one of the even numbers of SEQ ID NOS:1-2630, or a sequence having greater than 30% identity of any one of the even numbers of SEQ ID NOS:1-2630.

In some embodiments, the fusion protein further comprises a periplasmic secretion signal. In some embodiments, the periplasmic secretion signal is a periplasmic space targeting signal sequence derived from TorA, PelB, OmpA, pIII, PhoA, DsbA, TolB, TorT, a substrate of the Type II Secretion System (T2SS), or a functional variant thereof.

In some embodiments, the bacteriophage coat protein is fused to the lasso peptide component via a first linker. In some embodiments, the first linker is a cleavable linker. In some embodiments, the lasso peptide fragment comprises at least one unusual amino acid or unnatural amino acid.

In some embodiments, the fusion protein provided herein is encoded by a nucleic acid molecule. In some embodiments, the nucleic acid comprises a sequence of any one of the odd numbers of SEQ ID NOS:1-2630, or a sequence having greater than 30% identity of any one of the odd numbers of SEQ ID NOS:1-2630. In some embodiments, the nucleic acid molecule is a phagemid.

In some embodiments, the bacteriophage coat protein is derived from a filamentous bacteriophage, a polyhedral bacteriophage, a tailed bacteriophage, or a pleomorphic bacteriophage. In some embodiments, the bacteriophage coat protein is derived from an M13 phage, T4 phage, T7 phage or λ (lambda) phage.

In one aspect, provided herein are fusion proteins comprising at least one lasso peptide biosynthesis component fused to a secretion signal. In some embodiments, the secretion signal is a periplasmic secretion signal. In some embodiments, the periplasmic secretion signal is a periplasmic space targeting signal sequence derived from TorA, PelB, OmpA, pIII, PhoA, DsbA, TolB, TorT, a substrate of the Type II Secretion System (T2SS), or a functional variant thereof. In some embodiments, the secretion signal is an extracellular secretion signal. In some embodiments, the extracellular secretion signal is an extracellular space targeting signal sequence derived from HlyA, a substrate of the Type 1 Secretion System (TISS), or a functional variant thereof.

In some embodiments, the at least one lasso peptide biosynthesis component is a lasso peptidase, a lasso cyclase or a lasso RiPP Recognition Element (RRE). In some embodiments, the lasso peptidase comprises a sequence of any one of peptide Nos: 1316-2336, or a sequence having greater than 30% identity of any one of peptide Nos: 1316-2336. In some embodiments, the lasso cyclase comprises a sequence of any one of peptide Nos: 2337-3761, or a sequence having greater than 30% identity of any one of peptide Nos: 2337-3761. In some embodiments, the lasso RRE comprises a sequence of any one of peptide Nos: 3762-4593, or a sequence having greater than 30% identity of any one of peptide Nos: 3762-4593.

In some embodiments, the fusion protein comprises the lasso peptidase and the lasso RRE. In some embodiments, the fusion protein comprises a sequence of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562, or a sequence having greater than 30% identity of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562.

In some embodiments, the fusion protein comprises the lasso cyclase and the lasso RRE. In some embodiments, the fusion protein comprises a sequence selected from peptide Nos: 2504, 3608 or a sequence having greater than 30% identity of any one of peptide Nos: 2504 and 3608. In some embodiments, the fusion protein comprises the lasso peptidase and the lasso cyclase. In some embodiments, the fusion protein comprises a sequence having peptide No: 2903 or a sequence having greater than 30% identity thereof. In some embodiments, the fusion protein comprises the lasso peptidase, the lasso cyclase and the lasso RRE.

In some embodiments, the fusion protein comprises more than one lasso peptide biosynthesis component fused together via a first cleavable linker. In some embodiments, the lasso peptide biosynthesis component is fused to the secretion signal via a second cleavable linker.

In some embodiments, the fusion protein provided herein is encoded by a nucleic acid molecule. In some embodiments, the nucleic acid comprises a sequence of any one of the odd numbers of SEQ ID NOS:1-2630, or a sequence having greater than 30% identity of any one of the odd numbers of SEQ ID NOS:1-2630. In some embodiments, the nucleic acid molecule is a phagemid. In some embodiments, the nucleic acid comprises a sequence encoding any one of peptide Nos: 1316-2336, 2337-3761 and 3762-4593, or a peptide having greaterthan 30% sequence identity of any one of peptide Nos: 1316-2336, 2337-3761 and 3762-4593.

In one aspect, provided herein is a system comprising multiple nucleic acid sequences. Particularly, in some embodiments, the system comprising (i) a first nucleic acid sequence encoding one or more structural proteins of a bacteriophage; (ii) a second nucleic acid sequence encoding at least one lasso peptide component; and (iii) a third nucleic acid sequence encoding at least one lasso peptide biosynthesis component.

In some embodiments, the first nucleic acid sequence is one or more plasmid. In some embodiments, the bacteriophage is an M13 phage, a fd phage or a fl phage. In some embodiments, the first nucleic acid sequence encodes one or more of p3, p6, p7, p8 or p9 of filamentous phages, or a functional variant thereof.

In some embodiments, the third nucleic acid sequence encodes one or more fusion protein each comprising at least one lasso peptide biosynthesis component fused to a (a) first secretion signal or (b) purification tag. In some embodiments, the at least one lasso peptide biosynthesis component comprises one or more of a lasso peptidase, a lasso cyclase and a lasso RRE.

In some embodiments, the third nucleic acid sequence encodes a first fusion protein comprising a lasso peptidase and the (a) first secretion signal or (b) purification tag. In some embodiments, the third nucleic acid sequence further encodes a second fusion protein comprising a lasso cyclase and the (a) first secretion signal or (b) purification tag.

In some embodiments, the third nucleic acid sequence further encodes a third fusion protein comprising a lasso RRE and the (a) first secretion signal or (b) purification tag. In some embodiments, third nucleic acid sequence encodes a first fusion protein comprising a lasso peptidase, a lasso cyclase and the (a) first secretion signal or (b) purification tag. In some embodiments, the third nucleic acid sequence further encodes a second fusion protein comprising an RRE and the (a) first secretion signal or (b) purification tag.

In some embodiments, the third nucleic acid sequence encodes a first fusion protein comprising a lasso peptidase, a lasso RRE and the (a) first secretion signal or (b) purification tag. In some embodiments, the third nucleic acid sequence further encodes a second fusion protein comprising a lasso cyclase and the (a) first secretion signal or (b) purification tag.

In some embodiments, wherein the third nucleic acid sequence encodes a first fusion protein comprising a lasso cyclase, a lasso RRE and the (a) first secretion signal or (b) purification tag. In some embodiments, the third nucleic acid sequence further encodes a second fusion protein comprising a lasso peptidase and the (a) first secretion signal or (b) purification tag.

In some embodiments, the third nucleic acid sequence encodes a fusion protein comprising a lasso peptidase, a lasso cyclase, a lasso RRE and the (a) first secretion signal or (b) purification tag.

In some embodiments, the first secretion signal is a periplasmic secretion signal. In some embodiments, the first secretion signal is an extracellular secretion signal. In some embodiments, the third nucleic acid sequence is one or more plasmid. In some embodiments, the second nucleic acid sequence encodes a fourth fusion protein comprising a lasso peptide component, a bacteriophage coat protein and a second secretion signal, and wherein the second secretion signal is a periplasmic secretion signal. In some embodiments, the lasso peptide component is a lasso precursor peptide, a lasso core peptide, a lasso peptide or a functional fragment of lasso peptide.

In some embodiments, the lasso precursor peptide or the lasso core peptide is fused to the bacteriophage coat protein via a cleavable linker. In some embodiments, the bacteriophage coat protein comprises p3, p6, p8 or p9 of filamentous phages, or a functional variant thereof. In some embodiments, the second nucleic acid sequence is a plasmid or a phagemid.

In some embodiments, the second nucleic acid sequence comprises a sequence of (i) any one of the odd numbers of SEQ ID NOS:1-2630, (ii) a sequence having greater than 30% identity of any one of the odd numbers of SEQ ID NOS:1-2630, or (iii) a sequence encoding a polypeptide having greater than 30% identity of any one of the even numbers of SEQ ID NOS:1-2630.

In some embodiments, the third nucleic acid sequence comprises a sequence encoding a polypeptide having greater than 30% identify of any one of peptide Nos: 1316-2336, peptide Nos: 2337-3761, and peptide Nos: 3762-4593.

In some embodiments, two or more of the first nucleic acid sequence, the second nucleic acid sequence and the third nucleic acid sequence are in the same nucleic acid molecule. In some embodiments, the nucleic acid molecule is a phagemid.

In some embodiments, the periplasmic secretion signal is aperiplasmic space targeting signal sequence derived from TorA, PelB, OmpA, pIII, PhoA, DsbA, TolB, TorT, a substrate of the Type II Secretion System (T2SS), or a functional variant thereof. In some embodiments, the extracellular secretion signal is an extracellular space-targeting signal sequence derived from HlyA or a substrate of the Type 1 Secretion System (T1SS), or a functional variant thereof.

In some embodiments, the purification tag is Albumin-binding protein (ABP), Alkaline Phosphatase (AP), AU1 epitope, AU5 epitope, Bacteriophage 17 epitope (T7 tag), Bacteriophage V5 epitope (V5 tag), Biotin-carboxy carrier protein (BCCP), Bluetongue virus tag (B-tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S-transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1-tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

In some embodiments, the system further comprises a bacterial cell having an intracellular space, wherein the first and second nucleic acid sequences are in the intracellular space of the bacterial cell. In some embodiments, the third nucleic acid sequence is in the intracellular space of the bacterial cell. In some embodiments, the bacterial cell further comprises a periplasmic space, and wherein the at least one lasso peptide biosynthesis component encoded by the third nucleic acid sequence is in the periplasmic space or the extracellular space. In some embodiments, the third nucleic acid sequence is not in the intracellular space of the bacterial cell. In some embodiments, the bacterial cell is a cell of E. coli. In some embodiments, the lasso peptide fragment comprises at least one unusual amino acid or unnatural amino acid.

In one aspect, provided herein are non-naturally existing bacteriophages. In some embodiments, the phage comprises a first coat protein and a phagemid, wherein the first coat protein is fused to a lasso peptide component, and wherein the phagemid encodes at least a portion of the lasso peptide component. In some embodiments, the phagemid encodes a fusion protein comprising the first coat protein and the lasso peptide component. In some embodiments, the fusion protein further comprises a periplasmic secretion signal. In some embodiments, the fusion protein further comprises a cleavable linker.

In some embodiments, the first coat protein is p3, p6, p7, p8 or p9 of filamentous phages or a functional variant thereof. In some embodiments, the phagemid further encodes at least one lasso peptide biosynthesis component. In some embodiments, the phagemid encodes a fusion protein comprising the lasso peptide biosynthesis component and a secretion signal. In some embodiments, the secretion signal is a periplasmic secretion signal or an extracellular secretion signal. In some embodiments, the phagemid comprises a nucleic acid sequence of (i) any one of the odd numbers of SEQ ID NOS:1-2630, (ii) a sequence having greater than 30% identity of any one of the odd numbers of SEQ ID NOS:1-2630, or (iii) a sequence encoding a polypeptide having greater than 30% identify of any one of the even numbers of SEQ ID NOS:1-2630, peptide Nos: 1316-2336, peptide Nos: 2337-3761, and peptide Nos: 3762-4593.

In some embodiments, the phagemid further encodes at least one structural protein. In some embodiments, the at least one structural protein comprises p3, p6, p7, p8 or p9 of filamentous phages or a functional variant thereof. In some embodiments, the phage is an M13 phage. In some embodiments, the bacteriophage is in a culture medium of bacteria. In some embodiments, the culture medium thither comprises a bacterial host of the bacteriophage. In some embodiments, the culture medium thither comprises at least one lasso peptide biosynthesis component secreted by the bacterial host. In some embodiments, the bacterial host is E. coli. In some embodiments, the bacteriophage is purified.

In some embodiments, the bacteriophage is in contact with at least one lasso peptide biosynthesis component. In some embodiments, the at least one lasso peptide biosynthesis component is recombinantly produced or purified. In some embodiments, the lasso peptide component is a lasso precursor peptide and the at least one lasso biosynthesis component comprises a lasso peptidase and a lasso cyclase.

In some embodiments, the lasso peptide component is a lasso core peptide and the at least one lasso biosynthesis component comprises a lasso cyclase. In some embodiments, the lasso biosynthesis component thither comprises a lasso RRE. In some embodiments, two or more of the lasso peptidase, lasso cyclase and lasso RRE are fused together. In some embodiments, the lasso peptide component is a lasso peptide or a functional fragment of lasso peptide.

In some embodiments, the lasso peptide component comprises at least one unusual or unnatural amino acid. In some embodiments, the bacteriophage is a filamentous bacteriophage, a polyhedral bacteriophage, a tailed bacteriophage, or a pleomorphic bacteriophage.

In one aspect, provided herein are compositions comprising non-naturally existing bacteriophages. In some embodiments, the composition comprising at least two non-naturally existing bacteriophages according to any one of claims 73 to 96. In some embodiments, the lasso peptide components of the at least two non-naturally existing bacteriophages are the same. In some embodiments, each of the lasso peptide components of the at least two non-naturally existing bacteriophages is unique. In some embodiments, multiple bacteriophages as described herein are included in a phage display library.

In one aspect, provided herein are bacterial cells comprising the nucleic acid systems as described herein. In some embodiments, the bacterial cell is a cell of E. coli. In some embodiments, the bacterial cell is a cell of genetically engineered E. coli. In some embodiments, the genetically engineered E. coli cell comprises a nucleic acid sequence encoding a modified aminoacyl-tRNA synthetase (aaRS) capable of recognizing an unusual or unnatural amino acid residue. In some embodiments, the bacterial cell thither comprises a complementary tRNA that is aminoacylated by the modified aminoacyl-tRNA synthetase (aaRS). In some embodiments, the bacterial cell is included in a culture medium. In some embodiments, the culture medium comprises natural, non-natural or unusual amino acid residues.

In some embodiments, non-naturally existing bacteriophage described herein, or the composition described herein, or the bacteriophage display library described herein, or the bacterial cell described, or the cultural medium described herein, is in contact with a target molecule that is capable of binding to the lasso peptide component. In some embodiments, the target molecule is a cell surface protein or a secreted protein. In some embodiments, the cell surface protein comprises a transmembrane domain. In some embodiments, the cell surface protein does not comprise a transmembrane domain. In some embodiments, the target molecule is capable of modulating a cellular activity in a cell expressing the target molecule.

In one aspect, provided herein are methods for making a member of a bacteriophage display library. In some embodiments, the method comprises providing a system comprising (i) a first nucleic acid sequence encoding one or more structural proteins of a bacteriophage; (ii) a phagemid comprising a second nucleic acid sequence encoding a lasso peptide component fused to a bacteriophage coat protein; and (iii) a third nucleic acid sequence encoding at least one lasso peptide biosynthesis component; introducing the system into a population of bacterial cells; culturing the population of bacterial cells under a suitable condition to produce a plurality of bacteriophages each displaying the lasso peptide component on the coat protein; and wherein the lasso peptide biosynthesis component processes the lasso peptide component into a lasso peptide or a functional fragment of lasso peptide.

In some embodiments of the method, the bacterial cell comprises a periplasmic space, and wherein the lasso peptide component is fused to a first periplasmic secretion signal. In some embodiments, lasso peptide biosynthesis component is fused to a second periplasmic secretion signal; and wherein the lasso peptide biosynthesis component processes the lasso peptide component into the lasso peptide or functional fragment of lasso peptide in the periplasmic space. In some embodiments, the lasso peptide biosynthesis component is fused to an extracellular secretion signal; and wherein the lasso peptide biosynthesis component processes the lasso peptide component into the lasso peptide or functional fragment of lasso peptide in the extracellular space.

In one aspect, provided herein are methods for making a member of bacteriophage display library. In some embodiments, the method comprises providing a system comprising (i) a first nucleic acid sequence encoding one or more structural proteins of a bacteriophage; and (ii) a phagemid comprising a second nucleic acid sequence encoding a lasso peptide component fused to a bacteriophage coat protein; introducing the system into a population of bacterial cells; and culturing the population of bacterial cells under a first suitable condition to produce a plurality of bacteriophages each displaying the lasso peptide component on the coat protein; contacting the plurality of bacteriophages with at least one purified lasso peptide biosynthesis component under a second suitable condition to allow the lasso peptide biosynthesis component to process the lasso peptide component into a lasso peptide or functional fragment of lasso peptide.

In some embodiments, the plurality of bacteriophages are purified before the step of contacting. In some embodiments, the contacting is performed by adding a purified lasso peptide biosynthesis component into a culture medium containing the bacteriophages. In some embodiments, the population of bacterial cells are cells of E. coli as provided herein. In some embodiments, the lasso peptide components of the plurality of bacteriophages are the same. In some embodiments, each of the lasso peptide components of the plurality of bacteriophages is unique. In some embodiments, the system is the system as provided herein.

In one aspect, provided herein are methods for evolving a lasso peptide of interest for a target property. In some embodiments, the method comprises (a) providing a first bacteriophage display library comprising members derived from the lasso peptide of interest, wherein each member of the first lasso peptide display library comprises at least one mutation to the lasso peptide of interest; (b) subjecting the library to a first assay under a first condition to identify members having the target property; (c) identifying the mutations of the identified members as beneficial mutations; and (d) introducing the beneficial mutations into the lasso peptide of interest to provide an evolved lasso peptide.

In some embodiments, the method further comprises: (f) providing an evolved bacteriophage display library of lasso peptides comprising members derived from the evolved lasso peptide, wherein the members of the evolved bacteriophage display library retain at least one beneficial mutation; (g) repeating steps (b) through (d). In some embodiments, the method further comprises repeating steps f and g for at least one more round.

In some embodiments, the evolved bacteriophage display library is subjected to the first assay under a second condition more stringent for the target property than the first condition. In some embodiments, the evolved bacteriophage display library is subjected to a second assay to identify members having the target property. In some embodiments, the method further comprises validating the evolved lasso peptide using at least one additional assay different from the first or second assay.

In some embodiments, the target property comprises binding affinity for a target molecule. In some embodiments, the target property comprises binding specificity for a target molecule. In some embodiments, the target property comprises capability of modulating a cellular activity or cell phenotype. In some embodiments, the modulation is antagonist modulation or agonist modulation. In some embodiments, the mutation comprises substituting at least one amino acid with an unusual or unnatural amino acid. In some embodiments, the target property is at least two target properties screened simultaneously.

In one aspect, provided herein are methods for identifying a lasso peptide that specifically binds to a target molecule. In some embodiments, the method comprises providing a bacteriophage display library comprising a plurality of members, each member comprising a lasso peptide or a functional fragment of lasso peptide; contacting the library with the target molecule under a suitable condition that allows at least one member of the library to form a complex with the target molecule; and identifying the member of in the complex.

In some embodiments, the contacting is performed by contacting the library with the target molecule in the presence of a reference binding partner of the target molecule under a suitable condition that allows at least one member of the library to compete with the reference binding partner for binding to the target molecule; and wherein the identifying step is performed by detecting reduced binding of the reference binding partner to the target molecule; and identifying the member responsible for the reduced binding.

In some embodiments, the reference binding partner is a ligand for the target molecule. In some embodiments, the target molecule comprises one or more target sites, and the reference binding partner specifically binds to a target site of the target molecule. In some embodiments, the reference binding partner is a natural ligand or synthetic ligand for the target molecule. In some embodiments, the target molecule is at least two target molecules.

In one aspect, provided herein are methods for identifying a lasso peptide that modulates a cellular activity. In some embodiments, the method comprises (a) providing a bacteriophage display library comprising a plurality of members, each member comprising a lasso peptide or a functional fragment of lasso peptide; (b) subjecting the library to a suitable biological assay configured for measuring the cellular activity; (c) detecting a change in the cellular activity; and (d) identifying the members responsible for the detected change. In some embodiments, the step (b) is performed by subjecting the library to multiple biological assays configured for measuring the cellular activity; and the method further comprises selecting the members that have a high probability of being identified as responsible for the detected change in the cellular activity.

In one aspect, provided herein are methods for identifying an agonist or antagonist lasso peptide for a target molecule. In some embodiments, the method comprises providing a bacteriophage display library comprising a plurality of members, each member comprising a lasso peptide or a functional fragment of lasso peptide; contacting the library with a cell expressing the target molecule under a suitable condition that allows at least one member of the library to bind to the target molecule; measuring a cellular activity mediated by the target molecule; and identifying the member as an agonist ligand for the target molecule if said cellular activity is increased; or identifying the member as an antagonist ligand if said cellular activity is decreased.

In one aspect, provided herein is a nucleic acid molecule comprising a first sequence encoding one or more structural proteins of a bacteriophage and a second sequence encoding a first fusion protein comprising a lasso peptide component fused to a first coat protein of the bacteriophage. In some embodiments, the second sequence further encodes a second fusion protein comprising an identification peptide fused to a second coat protein of the bacteriophage. In some embodiments, the nucleic acid molecule is a mutated genome of the bacteriophage, wherein one or more endogenous sequence encoding the first and/or second coat protein(s) is deleted from the genome. In some embodiments, at least one of the first and second coat proteins is a nonessential outer capsid protein of the bacteriophage. In some embodiments, the second sequence is an exogenous sequence.

In some embodiments, the bacteriophage is a non-naturally occurring T4 phage, T7 phage or λ (lambda) phage. In some embodiments, the nucleic acid molecule is a mutated genome of the T4 phage with endogenous sequences coding for HOC and/or SOC deleted. In some embodiments, the second sequence encodes a fusion protein comprising the lasso peptide component fused to HOC. In some embodiments, the second sequence encodes a fusion protein comprising the identification peptide fused to SOC. In some embodiments, the lasso peptide component is a lasso precursor peptide, a lasso core peptide, a lasso peptide or a functional fragment of lasso peptide. In some embodiments, the lasso precursor peptide comprises a sequence of any one of the even numbers of SEQ ID NOS:1-2630, or a sequence having greater than 30% identity of any one of the even numbers of SEQ ID NOS:1-2630. In some embodiments, the nucleic acid comprises a sequence of any one of the odd numbers of SEQ ID NOS:1-2630, or a sequence having greater than 30% identity of any one of the odd numbers of SEQ ID NOS:1-2630.

In some embodiments, the identification peptide is a purification tag. In some embodiments, the purification tag is Albumin-binding protein (ABP), Alkaline Phosphatase (AP), AU1 epitope, AU5 epitope, Bacteriophage 17 epitope (T7 tag), Bacteriophage V5 epitope (V5 tag), Biotin-carboxy carrier protein (BCCP), Bluetongue virus tag (B-tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S-transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1 tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

In some embodiments, the first fusion protein further comprises a linker between the first protein and the lasso peptide component. In some embodiments, the linker is a cleavable linker.

In one aspect, provided herein are systems comprising multiple nucleic acid sequences. In some embodiments, the system comprising (i) a first nucleic acid sequence encoding one or more structural proteins of a bacteriophage; (ii) a second nucleic acid sequence encoding a first fusion protein comprising a lasso peptide component fused to a first coat protein of the bacteriophage; and (iii) a third nucleic acid sequence encoding at least one lasso peptide biosynthesis component. In some embodiments, the second nucleic acid sequence further encodes a second fusion protein comprising an identification peptide fused to a second coat protein of the bacteriophage.

In some embodiments, the first nucleic acid sequence does not encode the first and/or second nonessential outer capsid protein(s) of the bacteriophage. In some embodiments, the first nucleic acid sequence is a mutated genome of the bacteriophage. In some embodiments, the first nucleic acid sequence encodes the first and/or second coat protein(s) of the bacteriophage. In some embodiments, the first nucleic acid sequence is a wild-type genome of the bacteriophage. In some embodiments, at least one of the first and second coat proteins is a nonessential outer capsid protein of the bacteriophage.

In some embodiments, the bacteriophage is a non-naturally occurring T4 phage, T7 phage, or λ (lambda) phage. In some embodiments, the first nucleic acid sequence and the second nucleic acid sequence are in separate nucleic acid molecules. In some embodiments, comprising a site-specific recombinase capable of catalyzing homologous recombination between the first and second nucleic acid sequences to produce a recombinant sequence; wherein the recombinant sequence encodes for the one or more structural proteins of the bacteriophage and the first and/or second fusion protein.

In some embodiments, the mutated phage genome is T4 phage genome devoid of one or more sequence coding for the first and/or second nonessential outer capsid protein(s). In some embodiments, the second nucleic acid sequence is a plasmid. In some embodiments, the first nucleic acid sequence and the second nucleic acid sequence are in the same nucleic acid molecule. In some embodiments, the nucleic acid molecule is a mutated genome of the bacteriophage devoid of one or more endogenous sequence encoding the first and/or second nonessential outer capsid protein(s). In some embodiments, the second sequence is an exogenous sequence.

In some embodiments, the nucleic acid molecule is a mutated genome of the T4 phage with endogenous sequences coding for HOC and/or SOC deleted. In some embodiments, the second sequence encodes a fusion protein comprising the lasso peptide component fused to HOC. In some embodiments, the second sequence encodes a fusion protein comprising the identification peptide fused to SOC.

In some embodiments, the lasso peptide component is a lasso precursor peptide, a lasso core peptide, a lasso peptide or a functional fragment of lasso peptide.

In some embodiments, the lasso precursor peptide comprises a sequence of any one of the even numbers of SEQ ID NOS:1-2630, or a sequence having greater than 30% identity of any one of the even numbers of SEQ ID NOS:1-2630. In some embodiments, the nucleic acid comprises (i) a sequence of any one of the odd numbers of SEQ ID NOS:1-2630, (ii) a sequence having greater than 30% identity of any one of the odd numbers of SEQ ID NOS:1-2630, or (iii) a sequence encoding a polypeptide having greater than 30% identity of any one of the even numbers of SEQ ID NOS:1-2630.

In some embodiments, the third nucleic acid sequence encodes one or more lasso peptide biosynthesis component. In some embodiments, the at least one lasso peptide biosynthesis component comprises one or more of a lasso peptidase, a lasso cyclase and a lasso RRE. In some embodiments, the third nucleic acid sequence encodes a lasso peptidase. In some embodiments, the third nucleic acid sequence further encodes a lasso cyclase. In some embodiments, the third nucleic acid sequence further encodes a lasso RRE. In some embodiments, the third nucleic acid sequence encodes a fusion protein comprising a lasso peptidase and a lasso cyclase. In some embodiments, the third nucleic acid sequence further encodes a lasso RRE. In some embodiments, the third nucleic acid sequence encodes a fusion protein comprising a lasso peptidase and a lasso RRE. In some embodiments, the third nucleic acid sequence further encodes a lasso cyclase. In some embodiments, the third nucleic acid sequence encodes a fusion protein comprising a lasso cyclase and a lasso RRE. In some embodiments, the third nucleic acid sequence further encodes a lasso peptidase. In some embodiments, the third nucleic acid sequence encodes a fusion protein comprising a lasso peptidase, a lasso cyclase, and a lasso RRE.

In some embodiments, the third nucleic acid sequence comprises a sequence encoding a polypeptide having greater than 30% identify of any one of peptide Nos: 1316-2336, peptide Nos: 2337-3761, and peptide Nos: 3762-4593. In some embodiments, the third nucleic acid sequence is one or more plasmid.

In some embodiments, comprising a microbial cell having cytoplasm, wherein the first, second and third nucleic acid sequences are in the cytoplasm of the microbial cell. In some embodiments, the microbial cell is a bacterial cell or an archaea cell. In some embodiments, the bacterial cell is E. coli. In some embodiments, the system further comprises a cell-free biosynthesis reaction mixture, wherein the first, second and third nucleic acid sequence are in the cell-flee biosynthesis reaction mixture.

In some embodiments, the identification peptide is a purification tag. the purification tag is Albumin-binding protein (ABP), Alkaline Phosphatase (AP), AU1 epitope, AU5 epitope, Bacteriophage 17 epitope (17-tag), Bacteriophage V5 epitope (V5 tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S-transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1 tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G. In some embodiments, the first fusion protein further comprises a linker between the first protein and the lasso peptide component. In some embodiments, the liner is a cleavable linker.

In one aspect, provided herein is a system comprising a bacteriophage devoid of a first nonessential outer capsid protein, and a first fusion protein comprising a lasso peptide component fused to the first nonessential outer capsid protein of the bacteriophage. In some embodiments, the bacteriophage is devoid of a second nonessential outer capsid protein, and wherein the system further comprises a second fusion protein comprising an identification peptide fused to the second nonessential outer capsid protein of the bacteriophage.

In some embodiments, the bacteriophage comprises a mutated genome having one or more endogenous sequence encoding the first and/or second nonessential outer capsid protein(s) of the bacteriophage deleted. In some embodiments, the mutated genome further comprising an exogenous sequence encoding the first and/or second fusion protein.

In some embodiments, the bacteriophage is a non-naturally occurring T4 phage, T7 phage or λ (lambda) phage. In some embodiments, the bacteriophage is a non-naturally occurring T4 phage, and wherein the first nonessential outer capsid protein is HOC and the second nonessential outer capsid protein is SOC. In some embodiments, the lasso peptide component is a lasso precursor peptide, a lasso core peptide, a lasso peptide or a functional fragment of lasso peptide.

In some embodiments, the system further comprises at least one lasso peptide biosynthesis component. In some embodiments, the bacteriophage, the first and/or second fusion protein(s), and/or the at least one lasso peptide biosynthesis component is in a cytoplasm of the host microbial cell. In some embodiments, the bacteriophage, the first and/or second fusion protein(s), and/or the at least one lasso peptide biosynthesis component is in a cell-free biosynthesis reaction mixture. In some embodiments, the bacteriophage, the first and/or second fusion protein(s), and/or the at least one lasso peptide biosynthesis component is purified.

In some embodiments, the further comprises a solid support having at least one unique location, wherein the bacteriophage, the first and/or second fusion protein(s), and/or the at least one lasso peptide biosynthesis component is located at the unique location.

In some embodiments, the lasso precursor peptide comprises a sequence of any one of the even numbers of SEQ ID NOS:1-2630, or a sequence having greater than 30% identity of any one of the even numbers of SEQ ID NOS:1-2630.

In some embodiments, the at least one lasso peptide biosynthesis component comprises one or more of a lasso peptidase, a lasso cyclase and a lasso RRE. In some embodiments, the lasso peptidase comprises a sequence of any one of peptide Nos: 1316-2336, or a sequence having greater than 30% identity of any one of peptide Nos: 1316-2336. In some embodiments, the lasso cyclase comprises a sequence of any one of peptide Nos: 2337-3761, or a sequence having greater than 30% identity of any one of peptide Nos: 2337-3761. In some embodiments, the lasso RRE comprises a sequence of any one of peptide Nos: 3762-4593, or a sequence having greater than 30% identity of any one of peptide Nos: 3762-4593.

In some embodiments, the at least one lasso peptide biosynthesis component comprises a fusion protein comprising a lasso peptidase and a lasso cyclase. In some embodiments, the at least one lasso peptide biosynthesis component comprises a fusion protein comprising a lasso peptidase and a lasso RRE.

In some embodiments, the fusion protein comprising the lasso peptidase and the lasso RRE comprises a sequence of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562, or a sequence having greater than 30% identity of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562.

In some embodiments, the at least one lasso peptide biosynthesis component comprises a fusion protein comprising a lasso cyclase and a lasso RRE. In some embodiments, the fusion protein comprising the lasso cyclase and the lasso RRE comprises a sequence selected from peptide Nos: 2504, 3608 or a sequence having greater than 30% identity of any one of peptide Nos: 2504 and 3608.

In some embodiments, the at least one lasso peptide biosynthesis component comprises a fusion protein comprising a lasso peptidase and a lasso cyclase. In some embodiments, the fusion protein comprising the lasso peptidase and the lasso cyclase comprises a sequence having peptide No: 2903 or a sequence having greater than 30% identity thereof.

In some embodiments, the at least one lasso peptide biosynthesis component comprises a fusion protein comprising a lasso peptidase, a lasso cyclase, and a lasso RRE.

In some embodiments, the host microbial cell is a bacterial cell or an archaeal cell. In some embodiments, the host microbial cell is E. coli.

In some embodiments, the identification peptide is a purification tag. In some embodiments, the system further comprises a solid support having at least one unique location. In some embodiments, the purification tag is Albumin-binding protein (ABP), Alkaline Phosphatase (AP), AU1 epitope, AU5 epitope, Bacteriophage 17 epitope (17-tag), Bacteriophage V5 epitope (V5 tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S-transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1 tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

In some embodiments, the first fusion protein further comprises a linker between the first protein and the lasso peptide component. In some embodiments, the liner is a cleavable linker.

In one aspect, provided herein are non-naturally occurring bacteriophages. In some embodiments, the bacteriophage comprising a genome and a capsid, wherein the capsid comprises a plurality of a first coat proteins, and wherein at least one of the first coat proteins is fused to a lasso peptide component in a first fusion protein. In some embodiments, the phage further comprises a plurality of a second coat protein, and wherein at least one of the second coat protein is fused to an identification peptide in a second fusion protein.

In some embodiments, the genome is devoid of one or more endogenous sequence encoding the first and/or second coat protein(s). In some embodiments, the genome further comprises an exogenous sequence encoding the first and/or second fusion protein. In some embodiments, the genome is a wild-type genome. In some embodiments, at least one first coat protein is wild-type.

In some embodiments, at least one second coat protein is wild-type. In some embodiments, the genome is wild-type, and wherein the capsid comprises at least one first coat protein in the first fusion protein, and at least one first coat protein that is wild-type. In some embodiments, the capsid further comprises at least one second coat protein in the second fusion protein, and at least one second coat protein that is wild-type.

In some embodiments, the genome is devoid of an endogenous sequence coding for the first coat protein, and wherein the capsid comprises at least one first coat protein in the first fusion protein. In some embodiments, the genome further comprises an exogenous sequence encoding the first fusion protein. In some embodiments, the capsid further comprises at least one first coat protein that is wild-type. In some embodiments, the genome is further devoid of an endogenous sequence coding for the second coat protein, and wherein the capsid comprises at least one second coat protein in the second fusion protein. In some embodiments, the capsid further comprises at least one second coat protein that is wild-type. In some embodiments, the first coat protein is a nonessential outer capsid protein. In some embodiments, the second coat protein is a nonessential outer capsid protein.

In some embodiments, the bacteriophage is a non-naturally occurring T4 phage, T7 phage or a λ (lambda) phage. In some embodiments, the bacteriophage is a non-naturally occurring T4 phage, and wherein the first coat protein is HOC and the second coat protein is SOC. In some embodiments, the bacteriophage is capable of infection of a host microbial cell. In some embodiments, the host microbial organism is a bacterial cell or an archaea cell. In some embodiments, the host microbial organism is E. coli.

In some embodiments, the lasso peptide component is a lasso precursor peptide, a lasso core peptide, a lasso peptide or a functional fragment of lasso peptide. In some embodiments, the lasso precursor peptide comprises a sequence of any one of the even numbers of SEQ ID NOS:1-2630, or a sequence having greater than 30% identity of any one of the even numbers of SEQ ID NOS:1-2630.

In some embodiments, the bacteriophages as described herein are included in a library, wherein the first fusion proteins in the distinct members comprise distinct lasso peptide components. In some embodiments, the library further comprises a solid support comprising a plurality of unique locations, wherein each unique location contains a distinct member.

In one aspect, provided herein are methods for making a member of a bacteriophage display library. In some embodiments, the method comprises providing a system comprising (i) a first nucleic acid sequence encoding one or more structural proteins of a bacteriophage; (ii) a second nucleic acid sequence encoding a first fusion protein comprising a lasso peptide component fused to a first coat protein of the bacteriophage; and (iii) a third nucleic acid sequence encoding at least one lasso peptide biosynthesis component; introducing the system into a population of microbial cells or a cell-flee biosynthesis reaction mixture; incubating the population of microbial cells or the cell-flee biosynthesis reaction mixture under a suitable condition to produce a plurality of bacteriophages each displaying the lasso peptide component on the first coat protein; and wherein the lasso peptide biosynthesis component processes the lasso peptide component into a lasso peptide or a functional fragment of lasso peptide.

In some embodiments of the method, the first nucleic acid sequence comprises a mutated genome of the bacteriophage devoid of an endogenous sequence encoding the first coat protein. In some embodiments, the first nucleic acid sequence and the second nucleic acid sequence are in the same nucleic acid molecule. In some embodiments, the first, second and third nucleic acid sequences are in the same nucleic acid molecule. In some embodiments, the first nucleic acid sequence and the second nucleic acid sequence in different nucleic acid molecules that are configured to undergo homologous recombination to produce a recombinant sequence encoding the structural proteins and the first fusion protein. In some embodiments, the step of introducing the system into the population of microbial cells comprises infecting the population of microbial cells with a bacteriophage having a mutated genome comprising the first nucleic acid. In some embodiments, the step of introducing the system into the population of microbial cells comprises transfecting the population of microbial cells with one or more vectors comprising the second and/or third nucleic acid sequence.

In some embodiments of the method, the first nucleic acid comprises a mutated genome of the bacteriophage devoid of an endogenous sequence encoding a second coat protein of the bacteriophage, wherein the second nucleic acid sequence further encodes a second fusion protein comprising an identification peptide fused to the second coat protein; and wherein the step of incubating comprises incubating the population of microbial cells or cell-free biosynthesis reaction mixture under a suitable condition to produce a plurality of bacteriophages each displaying the lasso peptide component on the first coat protein and the identification peptide on the second coat protein.

In some embodiments, the method further comprises identifying the lasso peptide component based on the identification peptide. In some embodiments, the identification peptide is a purification tag, and the method further comprises purifying the produced plurality of bacteriophages.

In some embodiments of the methods, the first nucleic acid sequence comprises a wild-type genome of the bacteriophage. In some embodiments, the one or more structural proteins encoded by the first nucleic acid sequence comprises wild-type first coat protein. In some embodiments, the first and second nucleic acid sequences are in the same nucleic acid molecule.

In some embodiments of the method, the one or more structural proteins encoded by the first nucleic acid sequence further comprises a wild-type second coat protein; wherein the second nucleic acid sequence further encodes a second fusion protein comprising an identification peptide fused to the second coat protein; and wherein the step of incubating comprises incubating the population of microbial cells or cell-free biosynthesis reaction mixture under a suitable condition to produce a plurality of bacteriophages each comprising the wild-type second coat protein and the second fusion protein.

In some embodiments, the method further comprises identifying the lasso peptide component based on the identification peptide. In some embodiments, the identification peptide is a purification tag, and the method further comprises purifying the produced plurality of bacteriophages. In some embodiments, the first, second and third nucleic acid sequences are in the same nucleic acid molecule. In some embodiments, the nucleic acid molecule comprises a mutated genome of the bacteriophage. In some embodiments, the step of incubating is performed at a unique location configured to identify the lasso peptide component.

In some embodiments, the method further comprises identifying the lasso peptide component based on the unique location. In some embodiments, the bacteriophage is a non-naturally occurring T4 page, T7 phage or λ (lambda) phage. In some embodiments, the bacteriophage is a non-naturally occurring T4 page, and wherein the first coat protein is HOC and the second coat protein is SOC.

In one aspect, provided herein are methods for making a member of a bacteriophage display library. In some embodiments, the method comprises contacting a first bacteriophage devoid of a first nonessential outer capsid protein with a first fusion protein comprising a lasso peptide component fused to the first nonessential outer capsid protein of the bacteriophage under a suitable condition to produce a second bacteriophage displaying the lasso peptide component on the first coat protein.

In some embodiments of the methods, the first bacteriophage is further devoid of a second nonessential outer capsid protein, and wherein the method further comprises contacting the second bacteriophage with a second fusion protein comprising an identification peptide fused with the second nonessential outer capsid protein under a suitable condition to produce a third bacteriophage displaying the lasso peptide component on the first coat protein and the identification peptide on the second coat protein.

In some embodiments, the method further comprises contacting the second or the third bacteriophage with at least one lasso peptide biosynthesis component under a suitable condition to process the lasso peptide component into a lasso peptide or a functional fragment of lasso peptide. In some embodiments, the first bacteriophage comprises a mutated genome devoid of an endogenous sequence encoding the first nonessential outer capsid protein. In some embodiments, the first bacteriophage comprises a mutated genome devoid of an endogenous sequence encoding the second nonessential outer capsid protein. In some embodiments, the first bacteriophage comprises a mutated genome comprising an exogenous sequence encoding the first fusion protein. In some embodiments, the first bacteriophage comprises a mutated genome comprising an exogenous sequence encoding the second fusion protein. In some embodiments, the first bacteriophage comprises a wild-type genome of the bacteriophage. In some embodiments, the second or third bacteriophage is a non-naturally existing T4 phage, T7 phage or (lambda) phage. In some embodiments, the second or third bacteriophage is a non-naturally existing T4 phage, and wherein the first nonessential outer capsid protein is HOC, and the second nonessential outer capsid protein is SOC.

### 4. BRIEF DESCRIPTION OF THE FIGURES

The details of one or more embodiments of the present disclosure are set forth in the accompanying drawings and the description below. Other features, objects, and benefits of the present disclosure will be apparent from the description and drawings, and from the claims. All publications, patents and patent applications cited herein are hereby expressly incorporated by reference for all purposes.

The embodiments of the description described herein are not intended to be exhaustive or to limit the disclosure to the precise forms disclosed in the following drawings or detailed description. Rather, the embodiments are chosen and described so that others skilled in the art can appreciate and understand the principles and practices of the description.

FIG. 1 is a schematic illustration of the conversion of a lasso precursor peptide into a lasso peptide having the general structure 1 with the lariat-like topology.

FIG. 2 is a schematic illustration of a 26-mer linear core peptide corresponding to a lasso peptide.

FIG. 3 shows an exemplary system and process for producing a budding phage displaying a lasso peptide where the lasso formation occurs in the periplasmic space of the host cell of the phage.

FIG. 4 shows an exemplary system and process for producing a budding phage displaying a lasso peptide where the lasso formation occurs extracellularly to the host cell of the phage.

FIG. 5 shows an exemplary system and process for producing a budding phage displaying a lasso peptide where the lasso formation is catalyzed by contacting matured phage with purified lasso processing enzymes.

FIG. 6 shows exemplary methods for generation of a lytic phage particle displaying a lasso peptide, including genetic engineering of the lytic phage genome, or competitive assembly of T4 phage particles without genome editing.

FIG. 7 shows an exemplary system and method for producing lytic phage particles displaying a lasso peptide and a purification tag, where the phage assembly and lasso formation occurs in the cytoplasm of a host cell of the phage.

FIG. 8 shows an exemplary system and method for producing phage particles displaying a lasso peptide and a purification tag, where the phage assembly and lasso formation occurs in vitro in a cell-free system.

FIG. 9 shows an exemplary system and method for assembly fusion proteins containing a lasso peptide or a purification tag onto the capsid of a mutant T4 phage.

FIG. 10 shows exemplary methods for in vitro maturation of lasso peptide displayed on a mutant phage particle. Particularly, purified lasso peptide biosynthesis components are incubated with phage particles displaying a lasso precursor peptide under a condition suitable for lasso formation.

FIG. 11A and FIG. 11B show exemplary methods and systems for competitive assembly of T4 phage particles displaying a lasso peptide and a purification tag.

### 5. DETAILED DESCRIPTION

The features of the present disclosure are set forth specifically in the appended claims. A better understanding of the features and benefits of the present disclosure will be obtained by reference to the following detailed description that sets forth illustrative embodiments, in which the principles of the disclosure are utilized. To facilitate a full understanding of the disclosure set forth herein, a number of terms are defined below.

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (4th ed. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2nd ed. 2010). Molecular Biology of the Cell (6th Ed., 2014). Organic Chemistry, (Thomas Sorrell, 1999). March's Advanced Organic Chemistry (6^{th} ed. 2007). Lasso Peptides, (Li, Y.; Zirah, S.; Rebnffet, S., Springer; New York, 2015). Phage display—a powerful technique for immunotherapy (Bazan et al., Hum Vaccin Immunother. 2012, 8(12):1817-28) Engineering M13 for phage display (Sidhu S S., Biomol Eng. 2001, 18(2):57-63). T4 bacteriophage as a phage display platform (Gamkrelidze M. and Da̧browska K., Arch Microbiol. 2014, 196(7):473-9). Display of peptides and proteins on the surface of bacteriophage lambda (Sternberg N. and Hoess R H., Proc Natl Acad Sci USA. 1995, 92(5):1609-13); Phage Display in Biotechnology and Drug Discovery, 2^{nd} Ed., (Sidhu, S. S., Geyer, C. R. eds., CRC Press, New York, 2017).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

Generally, the nomenclature used herein and the laboratory procedures in organic chemistry, medicinal chemistry, molecular biology, microbiology, biochemistry, enzymology, computational biology, computational chemistry, and pharmacology described herein are those well-known and commonly employed in the art. Unless defined otherwise, all technical and scientific terms used herein generally have the same meaning as commonly understood by one of ordinary skill in the art to which this disclosure belongs. Methods and compounds of the present disclosure include those described generally above, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75^{th} Ed. General methods and principles of molecular biology and cloning are described in “Molecular Cloning: A Laboratory Manual”, 4^{th} edition, Michael R Green and Joseph Sambrook, Cold Spring Harbor Laboratory Press, 2012 and “Molecular Biology of the Cell”, 6^{th} Ed., Bruce Alberts, Alexander Johnson, Julian Lewis, David Morgan, Martin Raff, Keith Roberts, Peter Walter, Garland Science Press, 2014, the entire contents of which are hereby incorporated by reference. General methods and principles of phage display technology are described in “Phage Display in Biotechnology and Drug Discovery”, 2^{nd} Ed., Sidhu, S. S., Geyer, C. R. eds., CRC Press, New York, 2017, and “Phage Display of Peptides and Protein: A Laboratory Manual”, Kay, B. K. Winter, J., and McCafferty, J., Academic Press, New York, 1996. Additionally, general principles of organic chemistry are described in “Organic Chemistry”, Thomas Sorrell, University Science Books, Sausalito: 1999, and “March's Advanced Organic Chemistry”, 6^{th}Ed., Ed.: Smith, M. B. and March, J., John Wiley & Sons, New York: 2007, the entire contents of which are hereby incorporated by reference.

As used herein, the singular terms “a,” “an,” and “the” include the plural reference unless the context clearly indicates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, the term “naturally occurring” or “naturally existing” or “natural” or “native” when used in connection with biological materials such as nucleic acid molecules, polypeptides, bacteriophages, microbial host cells, oligonucleotides, amino acids, polypeptides, peptides, metabolites, small molecule natural products, host cells, and the like, refers to those that are found in or isolated directly from Nature and are not changed or manipulated by humans. The term “wild-type” refers to organisms, cells, genes, biosynthetic gene clusters, enzymes, proteins, oligonucleotides, and the like that are found in Nature and are unchanged relative to these components found in Nature (in the wild).

As defined herein, the term “natural product” refers to any product, a small molecule, organic compound, or peptide produced by living organisms, e.g., prokaryotes or eukaryotes, found in Nature, and which are produced through natural biosynthetic processes. As defined herein, “natural products” are produced through an organism's secondary metabolism or through biosynthetic pathways that are not essential for survival and not directly involved in cell growth and proliferation.

As used herein, the terms “non-naturally occurring” or “non-natural” or “unnatural” or “non-native” refer to a material, substance, molecule, cell, bacteriophage, enzyme, protein or peptide that is not known to exist or is not found in Nature or that has been structurally modified and/or synthesized by humans. The terms “non-natural” or “unnatural” or “non-naturally occurring” when used in reference to a microbial organism or microorganism or cell extract or gene or biosynthetic gene cluster of the present disclosure is intended to mean that the microbial organism (e.g., a phage) or derived cell extract or gene or biosynthetic gene cluster has at least one genetic alteration not normally found in a naturally occurring strain or a naturally occurring gene or biosynthetic gene cluster of the referenced species, including wild-type strains of the referenced species. Genetic alterations include, for example, introduction of expressible oligonucleotides or nucleic acids encoding polypeptides, other nucleic acid additions, nucleic acid deletions and/or other functional disruption of the microbial organism's genetic material. Such modifications include, for example, nucleotide changes, additions, or deletions in the genomic coding regions and functional fragments thereof, used for heterologous, homologous or both heterologous and homologous expression of polypeptides. Additional modifications include, for example, nucleotide changes, additions, or deletions in the genomic non-coding and/or regulatory regions in which the modifications alter expression of a gene or operon. Exemplary polypeptides include enzymes, proteins, or peptides within a lasso peptide biosynthetic pathway.

The terms “oligonucleotide” and “nucleic acid” refer to oligomers of deoxyribonucleotides (e.g., DNA) or ribonucleotides (e.g., RNA) and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A., et al., Nucleic Acid Res., 1991, 19, 5081-1585; Ohtsuka, E. et al., J. Biol. Chem., 1985, 260, 2605-2608; and Rossolini, G. M., et al., Mol. Cell. Probes, 1994, 8, 91-98). “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a lasso peptide of the present disclosure may include a bacterial and archaea host cells into which nucleic acids encoding the lasso peptide component have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “exogenous” as used herein with respect to a nucleic acid sequence in the genome of a bacteriophage is intended to mean that the referenced nucleic acid sequence is introduced into the phage genome. The molecule can be introduced to the phage genetic material, for example, via phage genetic cross, homologous recombination, DNA recombineering, CRISPR-Cas-mediated genetic engineering, genome fragment ligation, and de novo phage genome assembly (Pires et al., Microbiol Mol Biol Rev. 2016, 80(3):523-43). Such genetic engineering tools have aided the development of several display systems based on, e.g. T4, T7, or lambda (λ) phage for molecular evolution, such as affinity maturation of monoclonal antibodies and receptor ligands (Bazan et al., Hum Vaccin Immunother. 2012, 8(12):1817-28; Szardenings et al., J Biol Chem. 1997, 272(44):27943-8; Jiang et al., Infect Immun. 1997, 65(11):4770-7; Burgoon et al., J Immunol. 2001, 167(10):6009-14; Sternberg N. and Hoess R H., Proc Natl Acad Sci USA. 1995, 92(5):1609-13). Specifically, the term “exogenous” as it is used in reference to expression of an encoding nucleic acid refers to introduction of the encoding nucleic acid in an expressible form into the phage genome. The term “endogenous” as used herein with respect to a nucleic acid sequence in the genome of a bacteriophage is intended to refer to a referenced nucleic acid sequence that is present in the phage genome. Similarly, the term when used in reference to expression of an encoding nucleic acid refers to expression of an encoding nucleic acid contained by the phage genome.

An “isolated nucleic acid” is a nucleic acid, for example, an RNA, a DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially flee of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

As used herein, the term “biosynthetic gene cluster” refers to one or more nucleic acid molecule(s) independently or jointly comprising one or more coding sequences for a precursor and processing machinery capable of maturing the precursor into a biosynthetic end product. The coding sequences can comprise multiple open reading flames (ORFs) each independently coding for one component of the precursor and processing machinery. Alternatively, the coding sequences can comprise an ORF coding for two or more components of the precursor and processing machinery fused together, as further described herein. A biosynthetic gene cluster can be identified and isolated from the genome of an organism. Computer-based analytical tools can be used to mine genomic information and identify biosynthetic gene clusters encoding lasso peptides. For example, the genome-mining tool known as Rapid ORF Description and Evaluation Online (RODEO) has been used to identify more than a thousand of lasso biosynthetic gene clusters based on available genomic information (Tietz et al. Nat Chem Biol. 2017 May; 13(5): 470-478). Alternatively, a biosynthetic gene cluster can be assembled by artificially producing and combining the nucleic acid components of the gene cluster, using genetic manipulating methods and technology known in the art.

The term “amino acid” refers to naturally occurring and non-naturally occurring alpha-amino acids, as well as alpha-amino acid analogs and amino acid mimetics that function in a manner similar to the naturally occurring alpha-amino acids. Naturally encoded amino acids are the 22 common amino acids (alanine, arginine, asparagine, aspartic acid, cysteine, glutamine, glutamic acid. glycine, histidine, isoleucine, leucine, lysine, methionine, phenylalanine, proline, serine, threonine, tryptophan, tyrosine, valine, pyrrolysine and selenocysteine). Amino acid analogs or derivatives refers to compounds that have the same basic chemical structure as a naturally occurring amino acid, i.e., a carbon that is bound to a hydrogen, a carboxyl group, an amino group, and a side chain R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “non-natural amino acid” or “non-proteinogenic amino acid” or “unnatural amino acid” refer to alpha-amino acids that contain different side chains (different R groups) relative to those that appear in the twenty-two common or naturally occurring amino acids listed above. In addition, these terms also can refer to amino acids that are described as having D-stereochemistry, rather than L-stereochemistry of natural amino acids, despite the fact that some amino acids do occur in the D-stereochemical form in Nature (e.g., D-alanine and D-serine). Additional examples of non-natural amino acids are known in the art, such as those found in Hartman et al. PLoS One. 2007 Oct. 3; 2(10):e972; Hartman et al., Proc Natl Acad Sci USA. 2006 Mar. 21; 103(12):4356-61; and Fiacco et al. Chembiochem. 2016 Sep. 2; 17(17):1643-51.

The terms “polypeptide” and “protein” are used interchangeably herein to refer to a polymer of greater than about fifty (50) amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “peptide” as used herein refers to a polymer chain containing between two and fifty (2-50) amino acid residues. The terms apply to naturally occurring amino acid polymers as well as amino acid polymers in which one or more amino acid residues is a non-naturally occurring amino acid, e.g., an amino acid analog or non-natural amino acid.

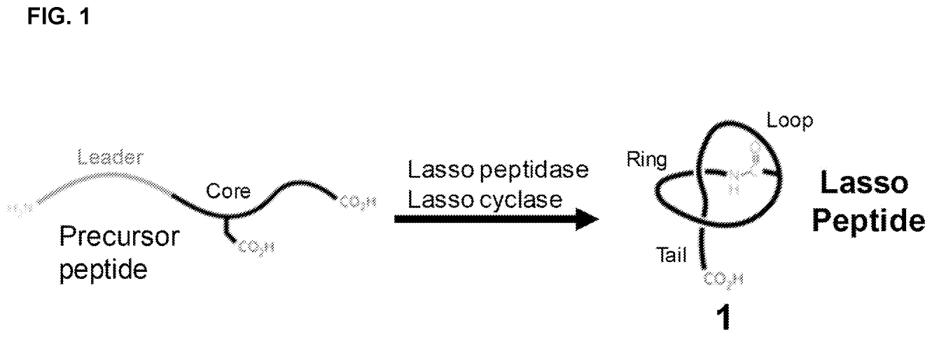

The terms “lasso peptide” and “lasso” are used interchangeably herein, and is used to refer to a class of peptide or polypeptide having the general lariat-like topology as exemplified in FIG. 1. As shown in the figure, the lariat-like topology can be generally divided into a ring portion, a loop portion, and a tail portion. Particularly, a region on one end of the peptide forms the ring around the tail on the other end of the peptide, the tail is threaded through the ring, and a middle loop portion connects the ring and the tail, together forming the lariat-like topology. Particularly, the amino acid residues that are joined together to form the ring are herein referred to as the “ring-forming amino acid.” A ring-forming amino acid can located at the N- or C-terminus of the lasso peptide (“terminal ring-forming amino acid”), or in the middle (but not necessarily the center) of a lasso peptide (“internal ring-forming amino acid”). The fragment of a lasso peptide between and including the two ring-forming amino acid residues is the ring portion; the fragment of a lasso peptide between the internal ring-forming amino acid and where the peptide threaded through the plane of the ring is the loop portion; and the remaining fragment of a lasso peptide starting from where the peptide is threaded through the plane of the ring is the tail portion. In addition to the lariat-like topology, additional topological features of a lasso peptide may further include intra-peptide disulfide bonding, such as disulfide bond(s) between the tail and the ring, between the ring and the loop, and/or between different locations within the tail. As used herein, “lasso peptide” or “lasso” refers to both naturally-existing peptides and artificially produced peptides that have the lariat-like topology as described herein. Similarly, “lasso peptide” or “lasso” also refers to analogs, derivatives, or variants of a lasso peptide, which analogs, derivatives or variants are also lasso peptides themselves.

The term “lasso precursor peptide” or “precursor peptide” as used herein refers to a precursor that is processed into or otherwise forms a lasso peptide. In some embodiments, a lasso precursor peptide comprises at least one a lasso core peptide portion. In some embodiments, a lasso precursor peptide comprises one or more amino acid residues or amino acid fragments that do not belong to a lasso core peptide, such as a leader sequence that facilitates recognition of the lasso precursor peptide by one or more lasso processing enzymes. In some embodiments, the lasso precursor peptide is enzymatically processed into a lasso peptide by removing the amino acid residues or fragments that do not belong to a lasso core peptide. In some embodiments, a lasso precursor peptide is the substrate of an enzyme that cleaves off the additional amino acid residues or fragments from a lasso precursor peptide to produce the lasso peptide. As used herein, the enzyme capable of catalyzing this reaction is referred to as the “lasso peptidase”.

The term “lasso core peptide” or “core peptide” refers to the peptide or the peptide segment of the precursor peptide that is processed into or otherwise forms a lasso peptide having the lariat-like topology. As used herein, a core peptide may have the same amino acid sequence as a lasso peptide, but has not matured to have the lariat-like topology of a lasso peptide. In various embodiments, core peptides can have different lengths of amino acid sequences. In some embodiments, the core peptide is at least about 5 amino acid long. In some embodiments, the core peptide is at least about 10 amino acid long. In some embodiments, the core peptide is at least about 11 amino acid long. In some embodiments, the core peptide is at least about 12 amino acid long. In some embodiments, the core peptide is at least about 13 amino acid long. In some embodiments, the core peptide is at least about 14 amino acid long. In some embodiments, the core peptide is at least about 15 amino acid long. In some embodiments, the core peptide is at least about 16 amino acid long. In some embodiments, the core peptide is at least about 17 amino acid long. In some embodiments, the core peptide is at least about 18 amino acid long. In some embodiments, the core peptide is at least about 19 amino acid long. In some embodiments, the core peptide is at least about 20 amino acid long. In some embodiments, the core peptide is at least about 25 amino acid long. In some embodiments, the core peptide is at least about 30 amino acid long. In some embodiments, the core peptide is at least about 35 amino acid long. In some embodiments, the core peptide is at least about 40 amino acid long. In some embodiments, the core peptide is at least about 45 amino acid long. In some embodiments, the core peptide is at least about 50 amino acid long. In some embodiments, the core peptide is at least about 55 amino acid long. In some embodiments, the core peptide is at least about 60 amino acid long. In some embodiments, the core peptide is at least about 65 amino acid long.

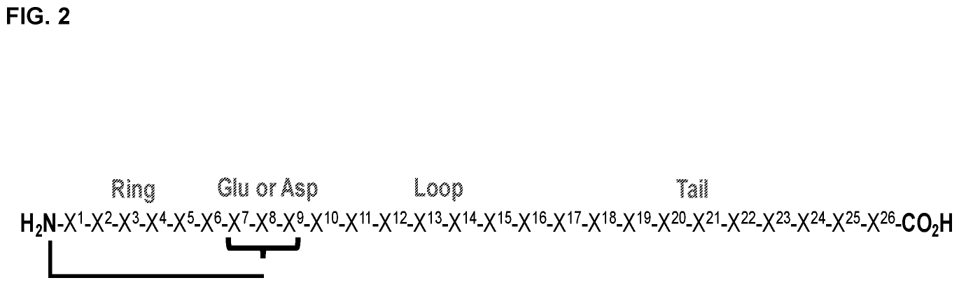

FIG. 2 shows an exemplary 26-mer linear lasso core peptide. Mutational analysis of the lasso precursor peptides McjA of microcin J25 and CapA of capistruin has revealed the high promiscuity of the biosynthetic machineries and the high plasticity of the lasso peptide structure, including the introduction of non-natural amino acids (See: Knappe, T. A., et al., Chem. Biol., 2009, 16, 1290-1298; Pavlova, O., et al. J. Biol. Chem., 2008, 283, 25589-25595; Al Toma, R S., et al., ChemBioChem, 2015, 16, 503-509). In addition, the feasible heterologous production of various variants in bacterial strains such as Escherichia coli and Streptomyces lividans indicates the relative ease of lasso peptide production. (See: Hegemann, J. D., et al., Biopolymers, 2013, 100, 527-542). The C-terminus of some lasso peptides has been shown to provide a source for diversification, for example through the formation of fusion peptides and proteins (See: Zong, C., et al., ACS Chem. Biol., 2016, 11, 61-68). Finally, the unique three-dimensional lariat-like topology of lasso peptides are difficult to achieve during chemical synthesis processes, but can be produced using a biosynthetically processes either in a host organism, or in a cell-flee biosynthesis system, having lasso precursors and lasso peptide biosynthetic enzymes.

Some naturally existing lasso peptides are encoded by a lasso peptide biosynthetic gene cluster, which typically comprises three main genes: one encodes for a lasso precursor peptide (referred to as Gene A), and two encode for processing enzymes including a lasso peptidase (referred to as Gene B) and a lasso cyclase (referred to as Gene C). The lasso precursor peptide comprises a lasso core peptide and additional peptidic fragments known as the “leader sequence” that facilitates recognition and processing by the processing enzymes. The leader sequence may determine substrate specificity of the processing enzymes. The processing enzymes encoded by the lasso peptide gene cluster convert the lasso precursor peptide into a matured lasso peptide having the lariat-like topology. Particularly, the lasso peptidase removes from the precursor peptide the additional portion that is not the lasso core peptide, and the lasso cyclase cyclize a terminal portion of the core peptide around a terminal tail portion to form the lariat-like topology.

Some lasso gene clusters further encodes for additional protein elements that facilitates the post-translational modification, including a facilitator protein known as the post-translationally modified peptide (RiPP) recognition element (RRE). A lasso peptide biosynthetic gene clusters may encode two or more of lasso peptidase, lasso cyclase and RRE as different domains in the same protein. Some lasso gene clusters further encodes for lasso peptide transporters, kinases, or proteins that play a role in immunity, such as isopeptidase. (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C. D., et al., Angew. Chem. Int. Ed., 2016, 55, 12717-12721; Zhu, S., et al., J. Biol. Chem. 2016, 291, 13662-13678).

As used herein, the term “lasso peptide component” refers to a protein comprising (i) a lasso peptide, (ii) a functional fragment of a lasso peptide, (iii) a lasso precursor peptide, or (iv) a lasso core peptide. As used herein, the term “lasso peptide biosynthesis component” refer to a protein comprising one or more of (i) a lasso peptidase, (ii) a lasso cyclase, and (iii) RRE.

Artificially produced lasso peptides may or may not be the same as a naturally-existing lasso peptide. For example, some artificially produced lasso peptides are non-naturally occurring lasso peptides. Some artificially produced lasso peptides can have a unique amino acid sequence and/or structure (e.g. lariat-like topology) that is different from those of any naturally-existing lasso peptide. Some artificially produced lasso peptides are analogs or derivatives of naturally-existing lasso peptides.

The terms “analog” and “derivative” are used interchangeably to refer to a molecule such as a lasso peptide, that have been modified in some fashion, through chemical or biological means, to produce a new molecule that is similar but not identical to the original molecule. For example, analogs or derivatives of a naturally-existing lasso peptide include a peptide or polypeptide that comprises an amino acid sequence of the naturally-existing lasso peptide, which has been altered by the introduction of amino acid residue substitutions, deletions, or additions. Analogs or derivatives of a naturally-existing lasso peptide also include a lasso peptide which has been chemically modified, e.g., by the covalent attachment of any type of molecule to the polypeptide. For example, but not by way of limitation, a lasso peptide may be chemically modified, e.g., by increase or decrease of glycosylation, acetylation, pegylation, phosphorylation, amidation, derivatization by known protecting/blocking groups, proteolytic cleavage, chemical cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. Further, a derivative of a lasso peptide, or a fragment of a lasso peptide may contain one or more non-classical or non-natural amino acids. A peptide or polypeptide derivative possesses a similar or identical function as a lasso peptide or a fragment of a lasso peptide. Analogs or derivatives also include a lasso peptide created by modifying the position of the ring-foaming nucleic acid residue in a lasso peptide sequence, while the remaining portions of the sequence unchanged. As used herein, an analog or derivative of a lasso peptide may but not necessarily have a similar amino acid sequence as the original lasso peptide. A peptide or polypeptide that has a similar amino acid sequence refers to a peptide or polypeptide that satisfies at least one of the followings: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a lasso peptide or a fragment of a lasso peptide; (b) a peptide of polypeptide encoded by a nucleotide sequence that hybridizes under stringent conditions to a nucleotide sequence encoding a lasso peptide or a fragment of a lasso peptide described herein of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al., Molecular Cloning: A Laboratory Manual (2001); and Maniatis et al., Molecular Cloning: A Laboratory Manual (1982)); or (c) a peptide or polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a lasso peptide or a fragment of a lasso peptide. A peptide or polypeptide with similar structure to a lasso peptide or a fragment of a lasso peptide refers to a peptide or polypeptide that has a similar secondary, tertiary, or quaternary structure of a lasso peptide or a fragment of a lasso peptide. The structure of a peptide or polypeptide can be determined by methods known to those skilled in the art, including but not limited to, X-ray crystallography, nuclear magnetic resonance, and crystallographic electron microscopy.

The term “variant” as used herein refers to a peptide or polypeptide comprising one or more (such as, for example, about 1 to about 25, about 1 to about 20, about 1 to about 15, about 1 to about 10, about 1 to about 5, or about 1 to about 3) amino acid sequence substitution, deletions, and/or additions as compared to a native or unmodified sequence. For example, a lasso peptide variant may result from one or more (such as, for example, about 1 to about 25, about 1 to about 20, about 1 to about 15, about 1 to about 10, about 1 to about 5, or about 1 to about 3) changes to an amino acid sequence of the native counterpart. Similarly, a phage protein variant may result from one or more (such as, for example, about 1 to about 25, about 1 to about 20, about 1 to about 15, about 1 to about 10, about 1 to about 5, or about 1 to about 3) changes to an amino acid sequence of the native counterpart.

Variants may be naturally occurring, such as allelic or splice variants, or may be artificially constructed. Polypeptide variants may be prepared from the corresponding nucleic acid molecules encoding the variants. In specific embodiments, the lasso peptide variant at least retains functionality of the native lasso peptide. For example, a variant of an antagonist lasso peptide. In specific embodiments, a lasso peptide variant binds to a target molecule and/or is antagonistic to the target molecule activity. In specific embodiments, a lasso peptide variant binds a target molecule and/or is agonistic to the target molecule activity. In certain embodiments, the variant is encoded by a single nucleotide polymorphism (SNP) variant of a nucleic acid molecule that encodes a lasso peptide, regions or sub-regions thereof, such as the ring, loop and/or tail portions of the lasso core peptide. In certain embodiments, variants of lasso peptides can be generated by modifying a lasso peptide, for example, by (i) introducing an amino acid sequence substitution or mutation, including the introduction of an unnatural or unusual amino acid, (ii) creating fragment of a lasso peptide; (iii) creating a fusion protein comprising one or more lasso peptides or fragment(s) of lasso peptides, and/or other non-lasso proteins or peptides, (iv) introducing chemical or biological transformation of the chemical functionality present in naturally-existing lasso peptides (e.g., inducing acylation, biotinylation, O-methylation, N-methylation, amidation, etc.), (v) making isotopic variants of naturally-existing lasso peptides, or any combinations of (i) to (v). For example, in one embodiment, one or more target-binding motif is introduced into a lasso peptide to provide a lasso peptide that specifically binds to a target molecule. For example, in some embodiments, a tripeptide Arg-Gly-Asp consists of Arginine, Glycine and Aspartate residues is introduced into a lasso peptide to create a lasso peptide variant that binds to a target integrin receptor. Artificially produced lasso peptides can be recombinantly produced using, for example, in vitro or in vivo recombinant expression systems, or synthetically produced.

The term “isotopic variant” when used in relation to a lasso peptide, refers to lasso peptides that contains an unnatural proportion of an isotope at one or more of the atoms that constitute such a peptide. In certain embodiments, an “isotopic variant” of a lasso peptide contains unnatural proportions of one or more isotopes, including, but not limited to, hydrogen (¹H), deuterium (²H), tritium (³H), carbon-11 (¹¹C), carbon-12 (¹²C) carbon-13 (¹³C), carbon-14 (¹⁴C), nitrogen-13 (¹³N) nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I) iodine-127 (¹²⁷I) iodine-129 (¹²⁹I) and iodine-131 (¹³¹I) In certain embodiments, an “isotopic variant” of a lasso peptide is in a stable form, that is, non-radioactive. In certain embodiments, an “isotopic variant” of a lasso peptide contains unnatural proportions of one or more isotopes, including, but not limited to, hydrogen (¹H) deuterium (²H), carbon-12 (¹²C), carbon-13 (¹³C), nitrogen-14 (¹⁴N), nitrogen-15 (¹⁵N), oxygen-16 (¹⁶O) oxygen-17 (¹⁷O), oxygen-18 (¹⁸O) fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a lasso peptide is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P) sulfur-35 (³⁵S), chlorine-36 (³⁶Cl), iodine-123 (¹²³I) iodine-125 (¹²⁵I), iodine-129 (¹²⁹I) and iodine-131 (¹³¹I). It will be understood that, in a lasso peptide as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a lasso peptide contains an unnatural proportion of deuterium. Unless otherwise stated, structures depicted herein are also meant to include lasso peptides that differ only in the presence of one or more isotopically enriched atoms from their naturally-existing counterparts. For example, lasso peptides having the present structures including the replacement of hydrogen by deuterium or tritium, or the replacement of a carbon by a ¹³C- or ¹⁴C-enriched carbon are within the scope of the present disclosure. Such lasso peptides are useful, for example, as analytical tools, as probes in biological assays, or as therapeutic agents in accordance with the present disclosure.

An “isolated” peptide or polypeptide (e.g., lasso peptide or a lasso processing enzyme) is substantially free of cellular material or other contaminating proteins from the cell or tissue source and/or other contaminant components from which the peptide or polypeptide is derived (such as culture medium of the host organism), or substantially free of chemical precursors or other chemicals when chemically synthesized. The language “substantially free” of cellular material or other contaminant components includes preparations of a peptide or polypeptide in which the peptide or polypeptide is separated from components of the cells from which it is isolated, recombinantly produced or biosynthesized. Thus, a peptide or polypeptide that is substantially free of cellular material includes preparations of lasso peptide having less than about 30%, 25%, 20%, 15%, 10%, 5%, or 1% (by dry weight) of heterologous protein (also referred to herein as a “contaminating protein”). In certain embodiments, when the peptide or polypeptide is recombinantly produced, it is substantially flee of culture medium, e.g., culture medium represents less than about 20%, 15%, 10%, 5%, or 1% of the volume of the protein preparation. In certain embodiments, when the peptide or polypeptide is produced by chemical synthesis, it is substantially free of chemical precursors or other chemicals, for example, it is separated from chemical precursors or other chemicals that are involved in the synthesis of the protein. In specific embodiments, where a lasso processing enzyme is produced by cell-free biosynthesis, it is substantially free of lasso precursors, other lasso processing enzymes, and/or in vitro TX-TL machinery in the cell flee biosynthesis system. Accordingly, such preparations of the lasso processing enzyme have less than about 30%, 25%, 20%, 15%, 10%, 5%, or 1% (by dry weight) of chemical precursors or compounds other than the lasso processing enzyme of interest. Contaminant components can also include, but are not limited to, materials that would interfere with activities for the lasso processing enzymes, and may include enzymes, hormones, and other proteinaceous or non-proteinaceous solutes. In certain embodiments, a peptide or polypeptide will be purified (1) to greater than 95% by weight of lasso peptide as determined by the Lowry method (Lowry et al., 1951, J. Bio. Chem. 193: 265-75), such as 96%, 97%, 98%, or 99%, (2) to a degree sufficient to obtain at least 15 residues of N terminal or internal amino acid sequence by use of a spinning cup sequenator, or (3) to homogeneity by SDS-PAGE under reducing or nonreducing conditions using Coomassie blue or silver stain. In specific embodiments, an isolated lasso processing enzyme includes the lasso processing enzyme in situ within recombinant cells since at least one component of the lasso processing enzyme natural environment will not be present. Ordinarily, however, isolated peptide and polypeptide will be prepared by at least one purification step. In specific embodiments, lasso peptides, or lasso precursors, one or more of lasso processing enzymes, co-factors, or a bacteriophage provided herein is isolated.

As used herein, the terms “in vitro transcription and translation” and “in vitro TX-TL” are used interchangeably and refer to a biosynthetic process outside an intact cell, where genes or oligonucleotides are transcribed into messenger ribonucleic acids (mRNAs), and mRNAs are translated into proteins or peptides. As used herein, the term “in vitro TX-TL machinery” refers to the components that act in concert to carry out the in vitro TX-TL. For the sole purpose of illustration, and by way of non-exhaustive and non-limiting examples, in some embodiments, an in vitro TX-TL machinery comprises enzyme(s) and co-factor(s) that carry out DNA transcription and/or mRNA translation. In some embodiments, an in vitro TX-TL machinery further comprises other small organic or inorganic molecules, such as amino acids, tRNAs or ATP, that facilitate the DNA transcription and/or mRNA translation. Various cellular components known to participate in in vivo transcription and translation can form part of the in vitro TX-TL machinery, see for example, Matsubayashi et al, “Purified cell-free systems as standard parts for synthetic biology.”; Curr Opin Chem Biol. 2014 October; 22:158-62; Li, et al. “Improved cell-free RNA and protein synthesis system.” PLoS One. 2014 Sep. 2; 9 (9):e106232. In some embodiments, different components can be provided individually and combined to assemble the in vitro TX-TL machinery. Exemplary ways of providing the in vitro TX-TL machinery components include recombinantly production, synthesis, and isolation from a cell. In some embodiments, the in vitro TX-TL machinery is provided in the form of one or more cell extract, or one or more supplemented cell extract that comprises the in vitro TX-TL machinery.

The terms “cell-free biosynthesis” and “CFB” are used interchangeably herein and refer to an in vitro (outside the cell) biosynthetic process for the production of one or more peptides or proteins. In some embodiments, cell-free biosynthesis occurs in a “cell-free biosynthesis reaction mixture” or “CFB reaction mixture” which provides various components, such as RNA, proteins, enzymes, co-factors, natural products, small molecules, organic molecules, to carry out protein synthesis outside a living cell. In some embodiments, the CFB reaction mixture can comprise one or more cell extracts or supplemented cell extracts, or commercially available cell-free reaction media (e.g. PURExpress®). Exemplary CFB methods and systems, including those involving the use of in vitro TX-TL, are described in Culler, S. et al., PCT Application WO2017/031399 A1, and is incorporated herein by reference.

Depending on the context, the term “condition suitable for lasso formation” may refer to, for example, a condition suitable for the expression of one or more protein products in a bacterial host (e.g., a lasso precursor peptide, or a processing enzyme). Exemplary suitable conditions included are not limited to a suitable culturing condition of the bacterial host that enable the protein synthesis and transportation in the host cell. Additionally or alternatively, depending on the context, the term “condition suitable for lasso formation” may refer to, for example, a condition suitable for post-translational modification of a lasso precursor peptide. Exemplary suitable conditions include but are not limited to a suitable temperature and/or incubation time for a lasso cyclase and/or lasso peptidase to process the lasso precursor in to a matured lasso peptide.

The term “display” and its grammatical variants, as used herein with respect to a chemical entity (e.g. a lasso peptide or functional fragment of lasso peptide), means to present or the presentation of the chemical entity (the “displayed entity”) in a manner so that it is chemically accessible in its environment and can be identified and/or distinguished from other chemical entities also present in the same environment. For example, a displayed entity can interact (e.g., bind to) or react (e.g. form covalent bonds) with other chemical entities (e.g., a target molecule) when the displayed entity is in contact with the other chemical entities. As disclosed herein, a displayed entity is affixed on a phage, where other components of the phage do not interfere with the chemical accessibility, activity, or reactivity intended for the displayed entity. For example, in certain embodiments, where the displayed entity is a lasso peptide for binding with a target protein (e.g., a cell surface protein), and/or modulating a biological activity of the target protein, then the phage capsid proteins are chemically inert with respect to the intended target binding or modulating activity of the lasso peptide.

“Bacteriophage” and “phage” are terms of art, and are used interchangeably to refer to a virus that infects and replicates within bacteria or archaea. Phages are composed of proteins that encapsulate a nucleic acid genome. Phages are classified by the International Committee on Taxonomy of Viruses (ICTV) according to morphology and nucleic acid, such as tailed phages, non-tailed phages, polyhedral phages, filamentous phages, and pleomorphic phages, DNA-containing phages, and RNA-containing phages, etc. Many phage species have been well-studied, and some are used as model organisms in various studies, such as a 186 phage, a λ phage, a Φ6 phage, a Φ29 phage, a ΦX 174, a G4 phage, an M13 phage, a fl phage, a fd phage, an MS2 phage, a N4 phage, a P1 phage, a P2 phage, a P4 phage, an RT7 phage, a T2 phage, a T4 phage, a T7 phage, or a T12 phage. Additional phage species can be found in Novik et al. in Antimicrobial research: Novel bioknowledge and educational programs; A. Mendex-Vilas, Ed.; pp. 251-259, 2017.

The term “structural protein” as used herein refers to one or more protein components of a phage that (i) form part of the protein capsid, (ii) facilitate packaging of the nucleic acid genome into the capsid, (iii) aid assembly of a phage particle, and/or (iv) for a budding phage, aid extrusion and budding of the phage particle, or for a lytic phage, aid lysis of the host cell. Exemplary phage structural proteins that can be used in connection with the present disclosure include but are not limited to protein p3, p4, p5, p6, p7, p8 and p9 of an M13 phage, and the protein components of a T4 phage, T7 phage or a X phage.

Particularly, a “coat protein” refers to a structural protein that locates on the surface of a phage, where at least a portion of the coat protein is chemically accessible in the environment containing the phage. Exemplary phage coat protein that can be used in connection with the present disclosure include but are not limited to protein p3, p6, p7, p8 and p9 of an M13 phage. A “nonessential outer capsid protein” refers to a phage coat protein that is nonessential for phage capsid assembly, and functional disruption and/or structural alteration of the protein does not affect phage productivity, viability, or infectivity. Examples of nonessential outer capsid proteins include but are not limited to HOC (highly antigenic outer capsid protein) and SOC (small outer capsid protein) of T4 phage. Other coat proteins that can be used for displaying a lasso peptide include but are not limited to pX of a T7 phage, pD or pV of a lambda (λ) phage (Bazan et al., Hum Vaccin Immunother. 2012, 8(12):1817-28), MS2 Coat Protein (CP) of an MS2 phage (Lino C A. et al., J Nanobiotechnology. 2017, 15(1):13), or the ΦX174 major spike protein G of a ΦX174 phage (Christakos K J. Virology. 2016, 488:242-8). Depending on the context, the term “bacteriophage” or “phage” as used herein may refer to a virus in its natural form or an artificially engineered version of the virus that is non-naturally existing.

The genome of a phage can be DNA- or RNA-based, and can encode as few as a handful of genes, or as many as hundreds of genes. According to the present disclosure, the genome of a phage may be genetically edited to encode more or less proteins as compared to its natural form, or to encode a variant, particularly a functional variant, of the natural phage protein. The term “functional variant” when used in connection with a phage protein refers to a protein that differs in the amino acid sequence from its natural counterpart, while retaining the function of the natural counterpart. For example, a functional variant of a bacteriophage coat protein retains the ability of assembly onto the surface of the phage where chemically accessible to agents present in the environment containing the phage. In exemplary embodiments, the functional variant of a coat protein can be a truncated version of the coat protein. In exemplary embodiments, the functional variant of a coat protein can be a fusion protein comprising a lasso peptide component fused to the coat protein or a variant thereof. In some embodiments, the genome of a phage is replaced by a phagemid. In some embodiments, a functional variant of protein or peptide has greater than 30% sequence identity of the protein or peptide. In various embodiments, a functional variant of a protein or a peptide can have greater than 30%, or greater than 40%, or greater than 50%, or greater than 60%, or greater than 70%, or greater than 880%, or greater than 90%, or greater than 95%, or greater than 99%, sequence identity to the protein or peptide.

“Phagemid” is also a term of art, and refers to a nucleic acid cloning vector that comprises a sequence encoding one or more proteins of interest as well as a sequence that signals for the packaging of the phagemid into a protein capsid of a phage. Proteins of the phage capsid that encapsulate the phagemid can be encoded by the phagemid itself or by one or more separate nucleic acid molecule. Proteins of the phage capsid and the packaging signal sequence of the phagemid can be derived from the same or distinct phage species. In some embodiments, the phagemid is packaged into the phage capsid in the form of a single-stranded (ss) nucleic acid molecule. In various embodiments, a phagemid can be a DNA-based vector or a RNA-based vector. For example, in some embodiments, a phagemid may contain an origin of replication from an fl phage (fl ori) that enables ssDNA replication and packaging into the phage capsid. In some embodiments, a phagemid may further contain an origin of replication derived from a bacterial double-stranded (ds) DNA plasmid that enables replication of dsDNA. In some embodiments, a phagemid can be used in combination with another vector encoding filamentous phage M13 structural proteins; the fl ori sequence enables packaging of the phagemid into an M13 phage capsid.

The term “display library” as used herein refers to the collection of a plurality of displayed entities, and each of the plurality of displayed entities in a library is a “member” of the library. To be clear, a “member” of the library refers to a unique displayed entity that is distinct from any other displayed entity(ies) that are present in the library. A library may comprise multiple identical copies of the same displayed entity, and the identical copies are collectively referred to as one member of the library. As used herein, two lasso peptides are considered “different” or “distinct” if they have different amino acid sequences or different structures (e.g., secondary, tertiary, or quaternary structure), or both different amino acid sequences and structures with respect to each other. For example, lasso cyclases having different selectivity for ring-forming amino acid residues can produce different lasso peptides from the same lasso core peptide by forming different ring structures.

Particularly, a “phage display library” is a collection of phages (e.g., filamentous phages), each phage comprising (i) at least one coat protein containing a lasso peptide component, and (ii) a nucleic acid molecule encoding at least a portion of the lasso peptide component. The coat protein is assembled on the surface of the phage where the lasso peptide component is chemically accessible to entities contacted with the phage. For example, the lasso peptide component can be a lasso precursor peptide or lasso core peptide capable of being processed into a matured lasso peptide or functional fragment of lasso peptide when contacted with one or more lasso biosynthesis components (e.g., lasso cyclase, lasso peptidase, and/or RRE). For another example, the lasso peptide component can be a lasso peptide or functional fragment of lasso peptide capable of binding to a target protein when contacted with the target protein.

A microbial cell (e.g., a bacteria or archaea cell) infected or susceptible to infection by a phage is referred to as the “host” of the phage.

“Periplasmic space” is a term of art and refers to the space between the inner cytoplasmic membrane and the bacterial outer membrane of a bacteria or archaea.

A “secretion signal” as used herein refers to a peptide, when becoming part of a protein, functions to direct transportation of the protein to a particular intracellular location or to the outside of the cell. A periplasmic secretion signal directs transportation of a protein containing the secretion signal to the periplasmic space. The transported protein can be soluble and floating in the periplasmic space, or can be attached to the inner cytoplasmic membrane. An extracellular secretion signal directs transportation of a protein containing the secretion signal to the outside of the cell. In some embodiments, the secretion signal peptide works in concert with other cellular proteins to effectuate the transportation. These other cellular proteins may be endogenously encoded by the cell's genome or exogenously introduced into the cell. In some embodiments, the secretion signal is removed from the transported protein after the transportation is completed or during the transportation process via endogenous or exogenous mechanisms.

The term “solid support” or “solid surface” means, without limitation, any column (or column material), plate (including multi-well plates), bead, test tube, microliter dish, solid particle (for example, agarose or sepharose), microchip (for example, silicon, silicon-glass, or gold chip), or membrane (for example, the membrane of a liposome or vesicle) to which a sample may be placed or affixed, either directly or indirectly (for example, through other binding partner intermediates such as antibodies).

The term “attached” or “associated” as used herein describes the interaction between or among two or more groups, moieties, compounds, monomers etc., e.g., a lasso peptide and a nucleic acid molecule. When two or more entities are “attached” to or “associated” with one another as described herein, they are linked by a direct or indirect covalent or non-covalent interaction. In some embodiments, the attachment is covalent. The covalent attachment may be, for example, but without limitation, through an amide, ester, carbon-carbon, disulfide, carbamate, ether, thioether, urea, amine, or carbonate linkage. The covalent attachment may also include a linker moiety, for example, a cleavable linker. Exemplary non-covalent interactions include hydrogen bonding, van der Waals interactions, dipole-dipole interactions, pi stacking interactions, hydrophobic interactions, magnetic interactions, electrostatic interactions, etc. Exemplary non-covalent binding pairs that can be used in connection with the present disclosure includes but are not limited to binding interaction between a ligand and its receptor, such as avidin or streptavidin and its binding moieties, including biotin or other streptavidin binding proteins.

The term “intact” as used herein with respect to a lasso peptide refers to the status of topologically intact. Thus, an “intact” lasso peptide is one comprising the complete lariat-like topology as described herein, including the terminal ring, middle loop and terminal tail. A sequence variant or a fragment of a lasso peptide may still be an intact lasso peptide, as long as the sequence variant or fragment of the lasso peptide still forms the lariat-like topology. For example, a lasso peptide having an amino acid residue truncated from its tail portion and another amino acid residue deleted from its ring portion may still form the lariat-like topology, even though the tail is shortened, and the ring is tightened. Such a variant is still considered an intact lasso peptide. In some embodiments, an intact lasso peptide has one or more effector functions.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In various embodiments, protein fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the protein. In a specific embodiment, a fragment of a protein retains at least 1, at least 2, at least 3, or more functions of the protein.

A “functional fragment,” “binding fragment,” or “target-binding fragment” of a lasso peptide retains some but not all of the topological features of an intact lasso peptide, while retaining at least one if not some or all of the biological functions attributed to the intact lasso peptide. The function comprises at least binding to or associating with a target molecule, directly or indirectly. For example, a functional fragment of a lasso peptide may retain only the ring structure without the loop and the tail (i.e., a head-to-tail cyclic peptide) or with an unthreaded tail loosely extended from the ring (i.e., a branched-cyclic peptide). In some embodiments, the loose tail may have the complete or partial amino acid sequence of the loop and tail portions of an intact lasso peptide. For example, lassomycin as described in Garvish et al. (Chem Biol. 2014 Apr. 24; 21(4): 509-518) is a functional fragment of lasso peptide that has the same amino acid sequence as lassomycin and the lariat-like topology. A functional fragment of a lasso peptide may only retain the ring and the loop structures without a tail portion. The various topologies assumed by functional fragments of lasso peptides are herein collectively referred to as the “lasso-related topologies.” Functional fragments of lasso peptides can be recombinantly produced in cells or produced via cell-flee biosynthesis as described further below.

As used herein, the term “contacting” and its grammatical variations, when used in reference to two or more components, refers to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting. For example, “contacting A with B and C” encompasses embodiments where A is first contacted with B then C, as well as embodiments where C is contacted with A then B, as well as embodiments where a mixture of A and C is contacted with B, and the like. Furthermore, such contacting does not necessarily require that the end result of the contacting process be a mixture including all of the referenced components, as long as at some point during the contacting process all of the referenced components are simultaneously present or simultaneously included in the same mixture or solution. Where one or more of the referenced components to be contacted includes a plurality (e.g., “contacting a library of candidate lasso peptides with the target molecule”), then each member of the plurality can be viewed as an individual component of the contacting process, such that the contacting can include contacting of any one or more members of the plurality with any other member of the plurality and/or with any other referenced component (e.g., some or all of the plurality of candidate lasso peptides can be contacted with a target molecule) in any order or combination.

The terms “target molecule” and “target protein” are used interchangeably herein and refer to a protein with which a lasso peptide binds under a physiological condition that mimics the native environment where the protein is isolated or derived from. As used herein, the target molecule is a cell surface protein or an extracellularly secreted protein. “Cell surface protein” is a term of art, and is used herein to refer to any protein that is known by the skilled person as a cell surface protein, and including those with any form of post-translational modifications, such as glycosylation, phosphorylation, lipidation, etc. In various embodiments, a cell surface protein can be a peptide or protein that has at least one part exposed to the extracellular environment, while embedded in or span the lipid layer of the cell membrane, or associated with a molecule integrated in the lipid layer. Exemplary types of cell surface proteins that can be used in connection with the present application include but are not limited to cell surface receptors, biomarkers, transporters, ion channels, and enzymes, where one particular protein may fit into one or more of these categories. In specific embodiments, cell surface protein is a cell surface receptor, such as a glucagon receptor, an endothelin receptor, an atrial natriuretic factor receptor, a G protein-coupled receptor (GPCR). In specific embodiments, cell surface protein is a cell surface ligand for a receptor, such as a PD-1 ligand (PD-L1 or PD-L2). In certain embodiments, a target molecule mediates one or more cellular activities (e.g., through a cellular signaling pathway), and as a result of the binding of a lasso peptide to the target molecule, the cellular activities are modulated. In some embodiments, a target molecule can be a protein secreted by a cell to the extracellular environment, such as growth factors, cytokines, etc.

The term “target site” as used herein refers to the amino acid residue or the group of amino acid residues with which a particular lasso peptide interacts to form the binding with the target molecule. According to the present disclosure, different lasso peptides may bind to different target sites or compete for binding with the same target site of a target molecule. In some embodiments, a lasso peptide specifically binds to a target molecule or a target site thereof.

The term “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single target-binding site of a binding protein and a single target site of a target molecule is the affinity of the binding protein or functional fragment for that target site. The ratio of dissociation rate (k_{off}) to association rate (kₒₙ) of a binding protein to a monovalent target site (k_{off}/kₒₙ) is the dissociation constant K_{D}, which is inversely related to affinity. The lower the K_{D} value, the higher the affinity of the antibody. The value of K_{D} varies for different complexes of lasso peptides or target proteins depends on both kₒₙ and k_{off}. The dissociation constant K_{D} for a binding protein (e.g., a lasso peptide) provided herein can be determined using any method provided herein or any other method well known to those skilled in the art. The affinity at one binding site does not always reflect the true strength of the interaction between a binding protein and the target molecule. When complex target molecule containing multiple, repeating target sites, such as a polyvalent target protein, come in contact with lasso peptides containing multiple target binding sites, the interaction of the lasso peptide with the target protein at one site will increase the probability of a reaction at a second site.

The terms “lasso peptides that specifically bind to a target molecule,” “lasso peptides that specifically bind to a target site,” and analogous terms are also used interchangeably herein and refer to lasso peptides that specifically bind to a target molecule, such as a polypeptide, or fragment, or ligand-binding domain. A lasso peptide that specifically binds to a target protein may bind to the extracellular domain or a peptide derived from the extracellular domain of the target protein. A lasso peptide that specifically binds to a target protein of a specific species origin (e.g., a human protein) may be cross-reactive with the target protein of a different species origin (e.g., a cynomolgus protein). In certain embodiments, a lasso peptide that specifically binds to a target protein of a specific species origin does not cross-react with the target protein from another species of origin.

A lasso peptide that specifically binds to a target protein can be identified, for example, by immunoassays (e.g., ELISA, fluorescent immunosorbent assay, chemiluminescence immune assay, radioimmunoassay (RIA), enzyme multiplied immunoassay, solid phase radioimmunoassay (SPRIA), a surface plasmon resonance (SPR) assay (e.g., Biacore®), a fluorescence polarization assay, a fluorescence resonance energy transfer (FRET) assay, Dot-blot assay, fluorescence activated cell sorting (FACS) assay, or other techniques known to those of skill in the art. A lasso peptide binds specifically to a target protein when it binds to the target protein with higher affinity than to any cross-reactive target molecule as determined using experimental techniques, such as radioimmunoassays (RIA) and enzyme linked immunosorbent assays (ELISAs). Typically a specific or selective reaction will be at least twice background signal or noise and may be more than 10 times background.

A lasso peptide which “binds a target molecule of interest” is one that binds the target molecule with sufficient affinity such that the lasso peptide is useful, for example, as a diagnostic or therapeutic agent in targeting a cell or tissue expressing the target molecule, and does not significantly cross-react with other molecules. In such embodiments, the extent of binding of the lasso peptide to a “non target” molecule will be less than about 10% of the binding of the lasso peptide to its particular target molecule, for example, as determined by fluorescence activated cell sorting (FACS) analysis or RIA.

With regard to the binding of a lasso peptide to a target molecule, the term “specific binding,” “specifically binds to,” or “is specific for” a particular polypeptide or an fragment on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding,” “specifically binds to,” or “is specific for” a particular polypeptide or a fragment on a particular polypeptide target as used herein refers to binding where a molecule binds to a particular polypeptide or fragment on a particular polypeptide without substantially binding to any other polypeptide or polypeptide fragment. In certain embodiments, a lasso peptide that binds to a target molecule has a dissociation constant (K_{D}) of less than or equal to 100 μM, 80 μM, 50 μM, 25 μM, 10 μM, 5 μM, 1 μM, 900 nM, 800 nM, 700 nM, 600 nM, 500 nM, 400 nM, 300 nM, 200 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM.

In the context of the present disclosure, a target protein is said to specifically bind or selectively bind to a lasso peptide, for example, when the dissociation constant (K_{D}) is <10⁻⁷ M. In some embodiments, the lasso peptides specifically bind to a target protein with a K_{D} of from about 10⁻⁷ M to about 10⁻¹² M. In certain embodiments, the lasso peptides specifically bind to a target protein with high affinity when the K_{D} is <10⁻⁸M or K_{D} is <10⁻⁹M. In one embodiment, the lasso peptides may specifically bind to a purified human target protein with a K_{D} of from 1×10⁻⁹ M to 10×10⁻⁹ M as measured by Biacore®. In another embodiment, the lasso peptides may specifically bind to a purified human target protein with a K_{D} of from 0.1×10⁻⁹ M to 1×10⁻⁹ M as measured by KinExA™ (Sapidyne, Boise, Id.). In yet another embodiment, the lasso peptides specifically bind to a target protein expressed on cells with a K_{D} of from 0.1×10⁻⁹M to 10×10⁻⁹M. In certain embodiments, the lasso peptides specifically bind to a human target protein expressed on cells with a K_{D} of from 0.1×10⁻⁹M to 1×10⁻⁹M. In some embodiments, the lasso peptides specifically bind to a human target protein expressed on cells with a K_{D} of 1×10⁻⁹M to 10×10⁻⁹M. In certain embodiments, the lasso peptides specifically bind to a human target protein expressed on cells with a K_{D} of about 0.1×10⁻⁹M, about 0.5×10⁻⁹M, about 1×10⁻⁹M, about 5×10⁻⁹M, about 10×10⁻⁹M, or any range or interval thereof. In still another embodiment, the lasso peptides specifically bind to a non-human target protein expressed on cells with a K_{D} of 0.1×10⁻⁹M to 10×10⁻⁹M. In certain embodiments, the lasso peptides specifically bind to a non-human target protein expressed on cells with a K_{D} of from 0.1×10⁻⁹M to 1×10⁻⁹M. In some embodiments, the lasso peptides specifically bind to a non human target protein expressed on cells with a K_{D} of 1×10⁻⁹M to 10×10⁻⁹M. In certain embodiments, the lasso peptides specifically bind to a non-human target protein expressed on cells with a K_{D} of about 0.1×10⁻⁹M, about 0.5×10⁻⁹M, about 1×10⁻⁹M, about 5×10⁻⁹M, about 10×10⁻⁹M, or any range or interval thereof.

“Binding affinity” generally refers to the strength of the sum total of noncovalent interactions between a single binding site of a molecule (e.g., a binding protein such as a lasso peptide) and its binding partner (e.g., a target protein). Unless indicated otherwise, as used herein, “binding affinity” refers to intrinsic binding affinity which reflects a 1:1 interaction between members of a binding pair (e.g., lasso peptide and target protein). The affinity of a binding molecule X for its binding partner Y can generally be represented by the dissociation constant (K_{D}). Affinity can be measured by common methods known in the art, including those described herein. Low-affinity lasso peptides generally bind target proteins slowly and tend to dissociate readily, whereas high-affinity lasso peptides generally bind target proteins faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the lasso peptide of interest and its target protein. The K_{D} or K_{D} value may also be measured by using surface plasmon resonance assays by Biacore®, using, for example, a Biacore® TM-2000 or a Biacore® TM-3000, or by biolayer interferometry using, for example, the Octet® QK384 system. An “on-rate” or “rate of association” or “association rate” or “kₒₙ” may also be determined with the same surface plasmon resonance or biolayer interferometry techniques described above using, for example, a Biacore® TM-2000 or a Biacore® TM-3000, or the Octet® QK384 system.

The term “compete” when used in the context of lasso peptides (e.g., a lasso peptide and other binding proteins that bind to and compete for the same target molecule or target site on the target molecule) means competition as determined by an assay in which the lasso peptide (or binding fragment) thereof under study prevents or inhibits the specific binding of a reference molecule (e.g., a reference ligand of the target molecule) to a common target molecule. Numerous types of competitive binding assays can be used to determine if a test lasso peptide competes with a reference ligand for binding to a target molecule. Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using I-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified target molecule bound to a solid surface, or cells bearing either of an unlabeled test target-binding lasso peptide or a labeled reference target-binding protein (e.g., reference target-binding ligand). Competitive inhibition may be measured by determining the amount of label bound to the solid surface in the presence of the test target-binding lasso peptide. Usually the test target-binding protein is present in excess. Target-binding lasso peptides identified by competition assay (e.g., competing lasso peptides) include lasso peptides binding to the same target site as the reference and lasso peptides binding to an adjacent target site sufficiently proximal to the target site bound by the reference for steric hindrance to occur. Additional details regarding methods for determining competitive binding are described herein. Usually, when a competing lasso peptide is present in excess, it will inhibit specific binding of a reference to a common target molecule by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

A “blocking” lasso peptide or an “antagonist” lasso peptide is one which inhibits or reduces biological activity of the target molecule it binds. For example, blocking lasso peptide or antagonist lasso peptide may substantially or completely inhibit the biological activity of the target molecule.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

The term “attenuate,” “attenuation,” or “attenuated,” when used herein, refers to partial (such as, 1%, 2%, 5%, 10%, 20%, 25%, 50%, 75%, 90%, 95%, 99%) or complete (i.e., 100%) reduction in a property, activity, effect, or value.

An “agonist” lasso peptide is a lasso peptide that triggers a response, e.g., one that mimics at least one of the functional activities of a polypeptide of interest (e.g., an agonist lasso peptide for glucagon-like peptide-1 receptor (GLP-1R) wherein the agonist lasso peptide mimics the functional activities of glucagon-like peptide-1). An agonist lasso peptide includes a lasso peptide that is a ligand mimetic, for example, wherein a ligand binds to a cell surface receptor and the binding induces cell signaling or activities via an intercellular cell signaling pathway and wherein the lasso peptide induces a similar cell signaling or activation. For the sole purpose of illustration, an “agonist” of glucagon-like peptide-1 receptor refers to a molecule that is capable of activating or otherwise increasing one or more of the biological activities of glucagon-like peptide-1 receptor, such as in a cell expressing glucagon-like peptide-1 receptor. In some embodiments, an agonist of glucagon-like peptide-1 receptor (e.g., an agonistic lasso peptide as described herein) may, for example, act by activating or otherwise increasing the activation and/or cell signaling pathways of a cell expressing a glucagon receptor protein, thereby increasing a glucagon-like peptide-1 receptor-mediated biological activity of the cell relative to the glucagon-like peptide-1 receptor-mediated biological activity in the absence of agonist.

The phrase “substantially similar” or “substantially the same” denotes a sufficiently high degree of similarity between two numeric values (e.g., one associated with a lasso peptide of the present disclosure and the other associated with a reference ligand) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference ligand.

The phrase “substantially increased,” “substantially reduced,” or “substantially different,” as used herein, denotes a sufficiently high degree of difference between two numeric values (e.g., one associated with a lasso peptide of the present disclosure and the other associated with a reference ligand) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference ligand.

As used herein, the term “modulating” or “modulate” refers to an effect of altering a biological activity (i.e. increasing or decreasing the activity), especially a biological activity associated with a particular biomolecule such as a cell surface receptor. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) of the compound for an inhibitor with respect to, for example, an enzyme.

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

The term “IC₅₀” refers to an amount, concentration, or dosage of a substance that is required for 50% inhibition of a maximal response in an assay that measures such response. The term “EC₅₀” refers to an amount, concentration, or dosage of a substance that is required for 50% of a maximal response in an assay that measures such response. The term “CC₅₀” refers an amount, concentration, or dosage of a substance that results in 50% reduction of the viability of a host. In certain embodiments, the CC₅₀ of a substance is the amount, concentration, or dosage of the substance that is required to reduce the viability of cells treated with the compound by 50%, in comparison with cells untreated with the compound. The term “Kₐ” refers to the equilibrium dissociation constant for a ligand and a protein, which is measured to assess the binding strength that a small molecule ligand (such as a small molecule drug) has for a protein or receptor, such as a cell surface receptor. The dissociation constant, K_{d}, is commonly used to describe the affinity between a ligand and a protein or receptor; i.e., how tightly a ligand binds to a particular protein or receptor, and is the inverse of the association constant. Ligand-protein affinities are influenced by non-covalent intermolecular interactions between the two molecules such as hydrogen bonding, electrostatic interactions, hydrophobic and van der Waals forces. The analogous term “Kᵢ” is the inhibitor constant or inhibition constant, which is the equilibrium dissociation constant for an enzyme inhibitor, and provides an indication of the potency of an inhibitor.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared. Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter off. Those skilled in the art will know what modifications can be made to the above parameters to either increase or decrease the stringency of the comparison, for example, and determine the relatedness of two or more sequences.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “vector” refers to a substance that is used to carry or include a nucleic acid sequence, including for example, a nucleic acid sequence encoding a lasso precursor peptide, or lasso processing enzymes as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both a lasso core peptide and a lasso cyclase), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a lasso precursor peptide as described herein), and it is further understood that expression levels can be optimized to obtain sufficient expression using methods well known in the art.

The term “identification peptide” as used herein refers to a peptide configured to identify a corresponding lasso peptide fragment. Various mechanisms of identification are contemplated. For example, in some embodiments, the identification peptide can produce a unique signal indicating the identity of the corresponding lasso peptide fragment. Thus, in some embodiments, the identification peptide can be a detectable probe or agent. In other embodiments, the identification peptide can enable specific isolation of the corresponding lasso peptide component from other components for further identification, characterization and/or use. In some embodiments, the identification peptide can be a purification tag. Other mechanisms of identification that are within the knowledge of those of ordinary skill in the art are also contemplated for the present disclosure.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as a complex between a lasso peptide and a target molecule as described herein, in a sample or subject. A detectable agent can be a substance that is capable of being visualized or a substance that is otherwise able to be determined and/or measured (e.g., by quantitation).

The term “purification tag” refers to any peptide sequence suitable for purification or identification of a polypeptide. The purification tag specifically binds to another moiety with affinity for the purification tag. Such moieties which specifically bind to a purification tag are usually attached to a matrix or a resin, such as agarose beads. Moieties which specifically bind to purification tags include antibodies, other proteins (e.g. Protein A or Streptavidin), nickel or cobalt ions or resins, biotin, amylose, maltose, and cyclodextrin. Exemplary purification tags include histidine (HIS) tags (such as a hexahistidine peptide), which will bind to metal ions such as nickel or cobalt ions. Other exemplary purification tags are the myc tag (EQKLISEEDL), the Strep tag (WSHPQFEK), the Flag tag (DYKDDDDK) and the V5 tag (GKPIPNPLLGLDST). The term “purification tag” also includes “epitope tags”, i.e., peptide sequences which are specifically recognized by antibodies. Exemplary epitope tags include the FLAG tag, which is specifically recognized by a monoclonal anti-FLAG antibody. The peptide sequence recognized by the anti-FLAG antibody consists of the sequence DYKDDDDK or a substantially identical variant thereof. In some embodiments, the polypeptide domain fused to the transposase comprises two or more tags, such as a SUMO tag and a STREP tag. The term “purification tag” also includes substantially identical variants of purification tags. “Substantially identical variant” as used herein refers to derivatives or fragments of purification tags which are modified compared to the original purification tag (e.g. via amino acid substitutions, deletions or insertions), but which retain the property of the purification tag of specifically binding to a moiety which specifically recognizes the purification tag. Additional exemplary purification tags that can be used in connection with the present disclosure include Albumin-binding protein (ABP), Alkaline Phosphatase (AP), AU1 epitope, AU5 epitope, Bacteriophage 17 epitope (T7 tag), Bacteriophage V5 epitope (V5-tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S-transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1 tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

### 5.3 Phage Display Library of Lasso Peptides and Methods of Making the Same

Provided herein are phage display libraries that comprises diversified species of lasso peptides or functional fragments of lasso peptides. In some embodiments, the library comprises a plurality of phage each expresses on its surface a coat protein, and the coat protein comprises a lasso peptide fragment. In some embodiments, the coat protein further comprises a non-lasso component having the amino acid sequence of a coat protein of the phage. In some embodiments, the coat protein comprises the lasso peptide component fused to non-lasso component. Particularly, in some embodiments, the lasso peptide component is fused to the non-lasso component via a cleavable linker, and upon cleavage of the linker, the lasso peptide component is severed from the phage.

According to the present disclosure, the lasso peptide fragment can assume the form of (i) an intact lasso peptide, (ii) a functional fragment of a lasso peptide, (iii) a lasso precursor peptide, or (iv) a lasso core peptide. A lasso peptide fragment can undergo transition among the different forms under a suitable condition. For example, when in contact with one or more lasso peptide biosynthesis component (e.g., a lasso peptidase, a lasso cyclase, and/or an RRE), a lasso peptide component in the form of a lasso precursor can be processed into the form of a lasso core peptide, and/or further processed into the form of an intact lasso peptide or a functional fragment of lasso peptide. In some embodiments, neither the non-lasso component of the coat protein nor other components of the phage interferes with either the functional or structural feature of the lasso peptide component.

According to the present disclosure, the amino acid sequence of the lasso peptide component can be encoded by a natural gene sequence (e.g., Gene A sequence of a lasso peptide biosynthesis gene cluster). In some embodiments, the lasso peptide component has the same amino acid sequence as a natural protein or peptide. Alternatively, the amino acid sequence of the lasso peptide component can be encoded by an artificially designed nucleic acid sequence that is non-naturally existing. In some embodiments, the lasso peptide component is a variant of a natural protein or peptide. Particularly, in some embodiments, one or more mutations can be introduced into the sequence of Gene A of a lasso peptide biosynthesis gene cluster to modify the coding sequence for a lasso peptide component. In some embodiments, the phage further comprises a nucleic acid molecule encoding at least part of the lasso peptide component displayed on the phage.

Protein and nucleic acid components of the phage display libraries, and methods and systems for producing the phage display library are described in further details below.

5.3.1 Lasso Peptides

As provided herein, an intact lasso peptide comprises the complete lariat-like topology as exemplified in FIG. 1. In some embodiments, the ring structure of a lasso peptide is formed through, for example, covalent bonding between a terminal amino acid residue and an internal amino acid residue. In some embodiments, the ring is formed via disulfide bonding between two or more amino acid residues of the lasso peptide. In alternative embodiments, the ring is formed via non-covalent interaction between two or more amino acid residues of the lasso peptide. In yet alternative embodiments, the ring is formed via both covalent and non-covalent interactions between at least two amino acid residues of the lasso peptide. In some embodiments, the ring is located at the C-terminus of the lasso peptide. In other embodiments, the ring is located at the N-terminus of the lasso peptide.

In specific embodiments, an N-terminal ring structure is formed by the formation of a bond between the N-terminal amino acid residue of the lasso peptide and an internal amino acid residue of the lasso peptide. In specific embodiment, an N-terminal ring structure is formed by formation of an isopeptide bond between the N-terminal amino group and the carboxyl group in the side chain of an internal amino acid residue, such as glutamate or aspartate residue, of the lasso peptide. In specific embodiments, an N-terminal ring structure is formed by the formation of an isopeptide bond between the N-terminal amino group and the carboxyl group in the side chain of an internal amino acid residue, such as glutamate or aspartate residue, located at the 6^{th} to 20^{th} position in the lasso peptide amino acid sequence, counting from its N terminus.

In specific embodiments, an N-terminal ring structure is formed by the formation of an isopeptide bond between the N-terminal amino group and the carboxyl group in the side chain of a glutamate located at the 6^{th} position in the lasso peptide amino acid sequence, counting from its N terminus, such that the lasso peptide has an N-terminal 6-member ring. In specific embodiments, an N-terminal ring structure is formed by the formation of an isopeptide bond between the N terminal amino group and the carboxyl group in the side chain of a glutamate located at the 7^{th} position in the lasso peptide amino acid sequence, counting from its N terminus, such that the lasso peptide has an N-terminal 7-member ring. In specific embodiments, an N-terminal ring structure is formed by the formation of an isopeptide bond between the N-terminal amino group and the carboxyl group in the side chain of a glutamate located at the 8^{th} position in the lasso peptide amino acid sequence, counting from its N terminus, such that the lasso peptide has an N-terminal 8-member ring. In specific embodiments, an N-terminal ring structure is formed by the formation of an isopeptide bond between the N-terminal amino group and the carboxyl group in the side chain of a glutamate located at the 9^{th} position in the lasso peptide amino acid sequence, counting from its N terminus, such that the lasso peptide has an N-terminal 9-member ring. In specific embodiments, an N-terminal ring structure is formed by the formation of an isopeptide bond between the N-terminal amino group and the carboxyl group in the side chain of a glutamate located at the 10^{th} position in the lasso peptide amino acid sequence, counting from its N terminus, such that the lasso peptide has an N-terminal 10-member ring. In specific embodiments, an N-terminal ring structure is formed by the formation of an isopeptide bond between the N-terminal amino group and the carboxyl group in the side chain of a glutamate located at the 11^{th} position in the lasso peptide amino acid sequence, counting from its N terminus, such that the lasso peptide has an N-terminal 11-member ring. In specific embodiments, an N-terminal ring structure is formed by the formation of an isopeptide bond between the N-terminal amino group and the carboxyl group in the side chain of a glutamate located at the 12^{th} position in the lasso peptide amino acid sequence, counting from its N terminus, such that the lasso peptide has an N-terminal 12-member ring. In specific embodiments, an N terminal ring structure is formed by the formation of an isopeptide bond between the N terminal amino group and the carboxyl group in the side chain of a glutamate located at the 13^{th} position in the lasso peptide amino acid sequence, counting from its N terminus, such that the lasso peptide has an N-terminal 13-member ring. In specific embodiments, an N terminal ring structure is formed by the formation of an isopeptide bond between the N-terminal amino group and the carboxyl group in the side chain of a glutamate located at the 14^{th} position in the lasso peptide amino acid sequence, counting from its N terminus, such that the lasso peptide has an N-terminal 14-member ring. In specific embodiments, an N terminal ring structure is formed by the formation of an isopeptide bond between the N-terminal amino group and the carboxyl group in the side chain of a glutamate located at the 15^{th} position in the lasso peptide amino acid sequence, counting from its N terminus, such that the lasso peptide has an N-terminal 15-member ring. In specific embodiments, an N terminal ring structure is formed by the formation of an isopeptide bond between the N-terminal amino group and the carboxyl group in the side chain of a glutamate located at the 16^{th} position in the lasso peptide amino acid sequence, counting from its N terminus, such that the lasso peptide has an N-terminal 16-member ring. In specific embodiments, an N terminal ring structure is formed by the formation of an isopeptide bond between the N-terminal amino group and the carboxyl group in the side chain of a glutamate located at the 17^{th} position in the lasso peptide amino acid sequence, counting from its N terminus, such that the lasso peptide has an N-terminal 17-member ring. In specific embodiments, an N terminal ring structure is formed by the formation of an isopeptide bond between the N-terminal amino group and the carboxyl group in the side chain of a glutamate located at the 18^{th} position in the lasso peptide amino acid sequence, counting from its N terminus, such that the lasso peptide has an N-terminal 18-member ring. In specific embodiments, an N terminal ring structure is formed by the formation of an isopeptide bond between the N-terminal amino group and the carboxyl group in the side chain of a glutamate located at the 19^{th} position in the lasso peptide amino acid sequence, counting from its N terminus, such that the lasso peptide has an N-terminal 19-member ring. In specific embodiments, an N terminal ring structure is formed by the formation of an isopeptide bond between the N-terminal amino group and the carboxyl group in the side chain of a glutamate located at the 20^{th} position in the lasso peptide amino acid sequence, counting from its N terminus, such that the lasso peptide has an N-terminal 20-member ring.

In specific embodiments, an N-terminal ring structure is formed by the formation of an isopeptide bond between the N-terminal amino group and the carboxyl group in the side chain of an aspartate located at the 6^{th} position in the lasso peptide amino acid sequence, counting from its N terminus, such that the lasso peptide has an N-terminal 6-member ring. In specific embodiments, an N terminal ring structure is formed by the formation of an isopeptide bond between the N terminal amino group and the carboxyl group in the side chain of an aspartate located at the 7^{th} position in the lasso peptide amino acid sequence, counting from its N terminus, such that the lasso peptide has an N-terminal 7-member ring. In specific embodiments, an N-terminal ring structure is formed by the formation of an isopeptide bond between the N-terminal amino group and the carboxyl group in the side chain of an aspartate located at the 8^{th} position in the lasso peptide amino acid sequence, counting from its N terminus, such that the lasso peptide has an N-terminal 8-member ring. In specific embodiments, an N-terminal ring structure is formed by the formation of an isopeptide bond between the N-terminal amino group and the carboxyl group in the side chain of an aspartate located at the 9^{th} position in the lasso peptide amino acid sequence, counting from its N terminus, such that the lasso peptide has an N-terminal 9-member ring. In specific embodiments, an N-terminal ring structure is formed by the formation of an isopeptide bond between the N-terminal amino group and the carboxyl group in the side chain of an aspartate located at the 10^{th} position in the lasso peptide amino acid sequence, counting from its N terminus, such that the lasso peptide has an N-terminal 10-member ring. In specific embodiments, an N-terminal ring structure is formed by the formation of an isopeptide bond between the N-terminal amino group and the carboxyl group in the side chain of an aspartate located at the 1 position in the lasso peptide amino acid sequence, counting from its N terminus, such that the lasso peptide has an N-terminal 11-member ring. In specific embodiments, an N-terminal ring structure is formed by the formation of an isopeptide bond between the N-terminal amino group and the carboxyl group in the side chain of an aspartate located at the 12^{th} position in the lasso peptide amino acid sequence, counting from its N terminus, such that the lasso peptide has an N-terminal 12-member ring. In specific embodiments, an N terminal ring structure is formed by the formation of an isopeptide bond between the N-terminal amino group and the carboxyl group in the side chain of an aspartate located at the 13^{th} position in the lasso peptide amino acid sequence, counting from its N terminus, such that the lasso peptide has an N-terminal 13-member ring. In specific embodiments, an N terminal ring structure is formed by the formation of an isopeptide bond between the N-terminal amino group and the carboxyl group in the side chain of an aspartate located at the 14^{th} position in the lasso peptide amino acid sequence, counting from its N terminus, such that the lasso peptide has an N-terminal 14-member ring. In specific embodiments, an N terminal ring structure is formed by the formation of an isopeptide bond between the N-terminal amino group and the carboxyl group in the side chain of an aspartate located at the 15^{th} position in the lasso peptide amino acid sequence, counting from its N terminus, such that the lasso peptide has an N-terminal 15-member ring. In specific embodiments, an N terminal ring structure is formed by the formation of an isopeptide bond between the N-terminal amino group and the carboxyl group in the side chain of an aspartate located at the 16^{th} position in the lasso peptide amino acid sequence, counting from its N terminus, such that the lasso peptide has an N-terminal 16-member ring. In specific embodiments, an N terminal ring structure is formed by the formation of an isopeptide bond between the N-terminal amino group and the carboxyl group in the side chain of an aspartate located at the 17^{th} position in the lasso peptide amino acid sequence, counting from its N terminus, such that the lasso peptide has an N-terminal 17-member ring. In specific embodiments, an N terminal ring structure is formed by the formation of an isopeptide bond between the N terminal amino group and the carboxyl group in the side chain of an aspartate located at the 18^{th} position in the lasso peptide amino acid sequence, counting from its N terminus, such that the lasso peptide has an N-terminal 18-member ring. In specific embodiments, an N terminal ring structure is formed by the formation of an isopeptide bond between the N terminal amino group and the carboxyl group in the side chain of an aspartate located at the 19^{th} position in the lasso peptide amino acid sequence, counting from its N terminus, such that the lasso peptide has an N-terminal 19-member ring. In specific embodiments, an N terminal ring structure is formed by the formation of an isopeptide bond between the N terminal amino group and the carboxyl group in the side chain of an aspartate located at the 20^{th} position in the lasso peptide amino acid sequence, counting from its N terminus, such that the lasso peptide has an N-terminal 20-member ring.

In specific embodiments, a C-terminal ring structure is formed by the formation of a bond between the C terminal amino acid residue of the lasso peptide and an internal amino acid residue of the lasso peptide. In specific embodiment, a C-terminal ring structure is formed by formation of an isopeptide bond between the C-terminal carboxyl group and the amino or amide group in the side chain of an internal amino acid residue, such as Asparagine, Glutamine or lysine residue, of the lasso peptide. In specific embodiments, a C-terminal ring structure is formed by the formation of an isopeptide bond between the C-terminal carboxyl group and the amino or amide group in the side chain of an internal amino acid residue, such as Asparagine, Glutamine or lysine residue, located at the 6^{th} to 20^{th} position in the lasso peptide amino acid sequence, counting from its C terminus.

As described herein, a lasso peptide can have one or more structural features that contribute to the stability of the lariat-like topology of the lasso peptide. In some embodiments, the ring is formed around the tail, which is threaded through the ring, and a middle loop portion connects the ring and the tail portions of the lasso peptide. In some embodiments, one or more disulfide bond(s) are formed (i) between the ring and tail portions, (ii) between the ring and loop portions, (iii) between the loop and tail portions; (iv) between different amino acid residues of the tail portion, or (v) any combination of (i) through (iv), which contribute to hold the lariat-like topology in place and increase the stability of the lasso peptide. In particular embodiments, one or more disulfide bonds are formed between the loop and the ring. In particular embodiments, one or more disulfide bonds are formed between the ring and the tail. In particular embodiments, one or more disulfide bonds are formed between the tail and the loop. In particular embodiments, one or more disulfide bonds are formed between different amino acid residues of the tail.

In particular embodiments, at least one disulfide bond is formed between the loop and ring portions of a lasso peptide, and at least one disulfide bond is formed between the tail and ring portions of a lasso peptide. In particular embodiments, at least one disulfide bond is formed between the loop and ring portions of a lasso peptide, and at least one disulfide bond is formed between the loop and tail portions of a lasso peptide. In particular embodiments, at least one disulfide bond is formed between the loop and ring portions of a lasso peptide, and at least one disulfide bond is formed between the different amino acid residues of the tail portion of a lasso peptide. In particular embodiments, at least one disulfide bond is formed between the tail and ring portions of a lasso peptide, and at least one disulfide bond is formed between the loop and tail portions of a lasso peptide. In particular embodiments, at least one disulfide bond is formed between the tail and ring portions of a lasso peptide, and at least one disulfide bond is formed between the different amino acid residues of the tail portion of a lasso peptide. In particular embodiments, at least one disulfide bond is formed between the loop and tail portions of a lasso peptide, and at least one disulfide bond is formed between the different amino acid residues of the tail portion of a lasso peptide. In particular embodiments, at least one disulfide bond is formed between the loop and ring portions of a lasso peptide, and at least one disulfide bond is formed between the tail and ring portions of a lasso peptide, and at least one disulfide bond is formed between the loop and tail portions of a lasso peptide. In particular embodiments, at least one disulfide bond is formed between the loop and ring portions of a lasso peptide, and at least one disulfide bond is formed between the tail and ring portions of a lasso peptide, an and at least one disulfide bond is formed between the different amino acid residues of the tail portion of a lasso peptide. In particular embodiments, at least one disulfide bond is formed between the loop and ring portions of a lasso peptide, and at least one disulfide bond is formed between the loop and tail portions of a lasso peptide, an and at least one disulfide bond is formed between the different amino acid residues of the tail portion of a lasso peptide. In particular embodiments, at least one disulfide bond is formed between the tail and ring portions of a lasso peptide, and at least one disulfide bond is formed between the loop and tail portions of a lasso peptide, an and at least one disulfide bond is formed between the different amino acid residues of the tail portion of a lasso peptide. In particular embodiments, at least one disulfide bond is formed between the loop and ring portions of a lasso peptide, and at least one disulfide bond is formed between the tail and ring portions of a lasso peptide, and at least one disulfide bond is formed between the loop and tail portions of a lasso peptide, and at least one disulfide bond is formed between the different amino acid residues of the tail portion of a lasso peptide.

In some embodiments, structural features of a lasso peptide that contribute to its topological stability comprise bulky side chains of amino acid residues located on the ring, the tail and/or the loop portion(s) of the lasso peptide, and these bulky side chains create an steric effect that holds the lariat-like topology in place. In some embodiments, the tail portion comprises at least one amino acid residue having a sterically bulky side chain. In some embodiments, the tail portion comprises at least one amino acid residue having a sterically bulky side chain that is located approximate to where the tail threads through the ring. In some embodiments, the amino acid residue having the sterically bulky side chain is located on the tail portion and is about 1, 2 or 3 amino acid residue(s) away from where the tail threads through the plane of the ring.

In some embodiments, the loop portion comprises at least one amino acid residue having a sterically bulky side chain that is located approximate to where the tail threads through the plane of the ring. In some embodiments, the amino acid residue having the sterically bulky side chain is located on the loop portion and is about 1, 2 or 3 amino acid residue(s) away from where the tail threads through the plane of the ring.

In some embodiments, the loop portion and the tail portion each comprises at least one amino acid residue having a sterically bulky side chain, and the bulky side chains from the tail and the loop portions flank the plane of the ring to hold the tail in position with respect to the ring. In some embodiments, the loop portion and the tail portion each comprises at least one amino acid residues having a sterically bulky side chain that is about 1, 2, 3 amino acid residue(s) away from where the tail threads through the plane of the ring.

In some embodiments, structural features of a lasso peptide that contribute to its topological stability comprise the size of the ring and the number of amino acid residues in the ring that have a sterically bulky side chain. Without being bound by the theory, it is contemplated that the larger the size of the ring is, the greater number of amino acid residues having sterically bulky side chains are needed to maintain topological stability of a lasso peptide. In some embodiments, a lasso peptide has a 6-member ring, and about 0 to about 3 amino acid residues in the ring that has a bulky side chain. In some embodiments, a lasso peptide has a 7-member ring, and about 0 to about 3 amino acid residues in the ring that has a bulky side chain. In some embodiments, a lasso peptide has an 8-member ring, and about 0 to about 4 amino acid residues in the ring that has a bulky side chain. In some embodiments, a lasso peptide has a 9-member ring, and about 0 to about 4 amino acid residues in the ring that has a bulky side chain.

In various embodiments, the amino acid residues having a sterically bulky side chain are natural amino acids, such as one or more selected from Proline (Pro), Phenylalanine (Phe), Tryptophan (Trp), Methionine (Met), Tyrosine (Tyr), Lysine (Lys), Arginine (Arg), and Histidine (His) residues. In some embodiments, the amino acid residues having a sterically bulky side chain can be unusual or unnatural amino acids, such as citrulline (Cit), hydroxyproline (Hyp), norleucine (Nle), 3-nitrotyrosine, nitroarginine, omithine (Om), naphtylalanine (Nal), Abu, DAB, methionine sulfoxide or methionine sulfone, and those commercially available or known to one of ordinary skill in the art.

According to the present disclosure, the size of ring, loop and/or tail portions of a lasso peptide can be variable. In certain embodiments, the ring portion has about 6 to about 20 amino acid residues including the two ring-forming amino acid residues. In certain embodiments, the loop portion has more than 4 amino acid residues. In certain embodiments, the tail portion has more than 1 amino acid residue.

5.3.2 Fusion Proteins

In one aspect, provided herein are fusion proteins comprising a lasso peptide component. In some embodiments, the fusion proteins are assembled into a phage, where the lasso peptide component is displayed on the surface of the capsid of the phage.

In various embodiments, the lasso peptide component of the fusion protein can be (i) an intact lasso peptide, (ii) a functional fragment of a lasso protein, (iii) a lasso precursor peptide; or (iv) a lasso core peptide. In some embodiments, the lasso peptide component of the fusion protein can undergo transition under a suitable condition among the different forms (i), (ii), (iii) and (iv).

In some embodiments, the lasso peptide component has the same amino acid sequence as a natural protein or peptide. In other embodiments, the lasso peptide component has an amino acid sequence that is a variant of a natural protein or peptide. Particularly, the lasso peptide component is a functional variant of a natural protein or peptide. Particularly, in some embodiments, the natural protein or peptide is a product of Gene A of a lasso peptide biosynthesis gene cluster.

In some embodiments, the lasso peptide component of the fusion protein has an amino acid sequence selected from the even numbers of SEQ ID NOS:1-2630. In some embodiments, the lasso peptide component of the fusion protein has an amino acid sequence that has greater than 30% sequence identity to any one of the even numbers of SEQ ID NOS:1-2630. Particularly, in some embodiments, the lasso peptide component of the fusion protein has an amino acid sequence that has greater than 40% sequence identity to any one of the even numbers of SEQ ID NOS:1-2630. In some embodiments, the lasso peptide component of the fusion protein has an amino acid sequence that has greater than 50% sequence identity to any one of the even numbers of SEQ ID NOS:1-2630. In some embodiments, the lasso peptide component of the fusion protein has an amino acid sequence that has greater than 60% sequence identity to any one of the even numbers of SEQ ID NOS:1-2630. In some embodiments, the lasso peptide component of the fusion protein has an amino acid sequence that has greater than 70% sequence identity to any one of the even numbers of SEQ ID NOS:1-2630. In some embodiments, the lasso peptide component of the fusion protein has an amino acid sequence that has greater than 80% sequence identity to any one of the even numbers of SEQ ID NOS:1-2630. In some embodiments, the lasso peptide component of the fusion protein has an amino acid sequence that has greater than 90% sequence identity to any one of the even numbers of SEQ ID NOS:1-2630. In some embodiments, the lasso peptide component of the fusion protein has an amino acid sequence that has greater than 95% sequence identity to any one of the even numbers of SEQ ID NOS:1-2630. In some embodiments, the lasso peptide component of the fusion protein has an amino acid sequence that has greater than 97% sequence identity to any one of the even numbers of SEQ ID NOS:1-2630. In some embodiments, the lasso peptide component of the fusion protein has an amino acid sequence that has greater than 99% sequence identity to any one of the even numbers of SEQ ID NOS:1-2630.

In some embodiments, the fusion protein further comprises a non-lasso component. Particularly, in some embodiments, the non-lasso component does not interfere with the functional and/or structural features of the lasso peptide component of the fusion protein. In some embodiments, the fusion protein retains one or more features of the lasso peptide component including (i) capability of transition from a lasso precursor peptide to a lasso core peptide when contacted with a lasso peptidase under a suitable condition; (ii) capability of transition from a lasso core peptide to an intact lasso peptide or a functional fragment of lasso peptide when in contact with a lasso cyclase; (iii) capability of binding to a target molecule of the lasso peptide or functional fragment of lasso peptide under a suitable condition; (iv) the lariat-like topology of an intact lasso peptide; (v) the lasso-related topologies of a functional fragment of lasso peptide. Exemplary suitable conditions include the condition for the lasso processing enzyme(s) to recognize its substrate and catalyze the reaction, or the presence of one or more cofactors of the lasso processing enzyme(s) such as RRE, or the condition suitable for a stand-alone lasso peptide (or functional fragment thereof) to bind to the target molecule, and those known to those of ordinary skill in the art.

In some embodiments, the fusion protein further comprises a phage structural protein or a functional variant thereof. In some embodiments, the phage structural protein is a coat protein which when assembled into the phage, is located on the surface of the phage capsid. In some embodiments, the orientation between the lasso peptide component and the phage coat protein in the fusion protein enables the lasso peptide component to be displayed on the surface of the phage.

According to the present disclosure, the phage coat protein can be derived from a phage that assembles new phage particles in the periplasmic space of the host cell, such as an M13 phage, a fl phage and a fd phage, and phages that assembles new phage particles in the cytosol of the host cell, such as a T4 phage, a T7 phage, a λ (lambda) phage, an MS2 phage, or a ΦX174 phage. Particularly, in some embodiments, the phage coat protein is derived from p3, p6, p7, p8 or p9 of filamentous phages. In other embodiments, the phage coat protein is derived from SOC (small outer capsid) protein or HOC (highly antigenic outer capsid) protein of a T4 phage, pX of a T7 phage, pD or pV of a λ (lambda) phage, MS2 Coat Protein (CP) of an MS2 phage, or the ΦX 174 major spike protein G of a ΦX 174 phage.

In some embodiments, the phage coat protein is a functional variant of a wild-type phage coat protein. Particularly, in some embodiments, the functional variant comprises one or more mutations to the wild-type phage coat protein, including but not limited to a deletion mutant (e.g., a truncation mutant), an insertion mutant, a missense mutant, a domain shuffling mutant, and a domain-swapping mutant.

In particular embodiments, the phage coat protein is derived from protein p3 of M13 phage. In some embodiment, the phage coat protein is a wild-type p3 protein. In other embodiments, the phage coat protein is a functional variant of the p3 protein that can be assembled onto the surface of a phage. Particularly, in some embodiments, the functional variant can be a truncated version of the p3 protein. In particular embodiments, the lasso peptide component is fused to the N terminus of the p3 protein or a functional variant thereof.

In particular embodiments, the phage coat protein is derived from a nonessential outer capsid protein of a phage, such as the SOC or HOC protein of the T4 phage, pX of a T7 phage, pD or pV of a λ (lambda) phage, MS2 Coat Protein (CP) of an MS2 phage, or the (DX 174 major spike protein G of a (DX 174 phage. In some embodiments, the phage coat protein is capable of assembly into a partially or fully assembled phage capsid.

In some embodiments, the lasso peptide component is fused to the non-lasso component of the fusion protein via a cleavable linker, such as an amino acid sequence comprising the cleavage site of a protease. Various cleavable linkers are known in the art. In some embodiments, when in contact with a suitable protease, the lasso peptide component is severed from the fusion protein. In particular embodiments, contacting a population of phage with a suitable protease can sever the lasso peptide component from the phage.

In some embodiments, the fusion protein further comprises a secretion signal that enables transportation of the fusion protein into a particular intracellular location or outside of a cell comprising the fusion protein. In some embodiments, the secretion signal directs the fusion protein to an intracellular location wherein the fusion protein is assembled into a phage. In some embodiments, a wild type version of the coat protein can compete with a fusion protein comprising the coat protein for assembly into a phage capsid. In some embodiments, a wild type version of the nonessential outer capsid protein can compete with a fusion protein comprising the nonessential outer capsid protein for assembly into a phage capsid.

In some embodiments, the secretion signal is a periplasmic secretion signal. In some embodiments, the secretion signal is an extracellular secretion signal. In some embodiments, the fusion protein comprising a periplasmic secretion signal is transported into the periplasmic space where the fusion protein is assembled into a phage. In some embodiments, the fusion protein is associated with the inner cytoplasmic membrane. In some embodiments, the lasso peptide component of the fusion protein is in the periplasmic space, wherein the lasso peptide component is processed to become an intact lasso peptide or a functional fragment of lasso peptide. In some embodiments, the secretion signal is removed from the fusion protein after the fusion protein arrives at the destination. In some embodiments, the secretion signal is fused at the N terminal end of the fusion protein. In some embodiments, the secretion signal is fused at the C-terminal end of the fusion protein. Exemplary periplasmic secretion signals that can be used in connection with the present disclosure include but are not limited to a periplasmic space-targeting signal sequence derived from TorA, PelB, OmpA, pIII, PhoA, DsbA, TolB, TorT, a substrate of the Type II Secretion System (T2SS), or a functional variant thereof. Exemplary extracellular secretion signals that can be used in connection with the present disclosure include but are not limited to an extracellular space targeting signal sequence derived from HlyA, a substrate of the Type 1 Secretion System (T1SS), or a functional variant thereof.

In another aspect, provided herein fusion proteins comprising at least one lasso peptide biosynthesis component. According to the present disclosure, the lasso peptide biosynthesis component can comprise (i) a lasso peptidase, (ii) a lasso cyclase, (iii) an RRE, or any combination of (i) to (iii). In some embodiments, the fusion protein comprises one or more of a lasso peptidase, a lasso cyclase and an RRE. In particular embodiments, the fusion protein comprise a lasso peptidase. In other embodiments, the fusion protein comprises a lasso cyclase. In other embodiments, the fusion protein comprises an RRE. In other embodiments, the fusion protein comprises a lasso peptidase fused with a lasso cyclase. In other embodiments, the fusion protein comprises a lasso peptidase fused with an RRE. In other embodiments, the fusion protein comprises a lasso cyclase fused with an RRE. In yet other embodiments, the fusion protein comprises a lasso peptidase, a lasso cyclase and an RRE fused together.

In some embodiments, the lasso peptide biosynthesis component has the same amino acid sequence as a natural protein or peptide. In other embodiments, the lasso peptide biosynthesis component has an amino acid sequence that is a variant of a natural protein or peptide. Particularly, the lasso peptide biosynthesis component is a functional variant of a natural protein or peptide. In some embodiments, the natural protein or peptide is a product of a gene of a lasso peptide biosynthesis gene cluster. Particularly, in some embodiments, the natural protein or peptide is a product of Gene B of a lasso peptide biosynthesis gene cluster. Particularly, in some embodiments, the natural protein or peptide is a product of Gene C of a lasso peptide biosynthesis gene cluster.

In some embodiments, the lasso peptide biosynthesis component of the fusion protein comprises the sequences of a lasso peptidase or a functional variant thereof. Particularly, the lasso peptide biosynthesis component of the fusion protein has an amino acid sequence selected from peptide Nos: 1316-2336. In some embodiments, the lasso peptide biosynthesis component of the fusion protein has an amino acid sequence that has greater than 30% sequence identity to any one of peptide Nos: 1316-2336. In some embodiments, the lasso peptide biosynthesis component of the fusion protein has an amino acid sequence that has greater than 40% sequence identity to any one of peptide Nos: 1316-2336. In some embodiments, the lasso peptide biosynthesis component of the fusion protein has an amino acid sequence that has greater than 50% sequence identity to any one of peptide Nos: 1316-2336. In some embodiments, the lasso peptide biosynthesis component of the fusion protein has an amino acid sequence that has greater than 60% sequence identity to any one of peptide Nos: 1316-2336. In some embodiments, the lasso peptide biosynthesis component of the fusion protein has an amino acid sequence that has greater than 70% sequence identity to any one of peptide Nos: 1316-2336. In some embodiments, the lasso peptide biosynthesis component of the fusion protein has an amino acid sequence that has greater than 80% sequence identity to any one of peptide Nos: 1316-2336. In some embodiments, the lasso peptide biosynthesis component of the fusion protein has an amino acid sequence that has greater than 90% sequence identity to any one of peptide Nos: 1316-2336. In some embodiments, the lasso peptide biosynthesis component of the fusion protein has an amino acid sequence that has greater than 95% sequence identity to any one of peptide Nos: 1316-2336. In some embodiments, the lasso peptide biosynthesis component of the fusion protein has an amino acid sequence that has greater than 99% sequence identity to any one of peptide Nos: 1316-2336.

In some embodiments, the lasso peptide biosynthesis component of the fusion protein comprises the sequences of a lasso cyclase or a functional variant thereof. Particularly, the lasso peptide biosynthesis component of the fusion protein has an amino acid sequence selected from peptide Nos: 2337-3761. In some embodiments, the lasso peptide biosynthesis component of the fusion protein has an amino acid sequence that has greater than 30% sequence identity to any one of peptide Nos: 2337-3761. In some embodiments, the lasso peptide biosynthesis component of the fusion protein has an amino acid sequence that has greater than 40% sequence identity to any one of peptide Nos: 2337-3761. In some embodiments, the lasso peptide biosynthesis component of the fusion protein has an amino acid sequence that has greater than 50% sequence identity to any one of peptide Nos: 2337-3761. In some embodiments, the lasso peptide biosynthesis component of the fusion protein has an amino acid sequence that has greater than 60% sequence identity to any one of peptide Nos: 2337-3761. In some embodiments, the lasso peptide biosynthesis component of the fusion protein has an amino acid sequence that has greater than 70% sequence identity to any one of peptide Nos: 2337-3761. In some embodiments, the lasso peptide biosynthesis component of the fusion protein has an amino acid sequence that has greater than 80% sequence identity to any one of peptide Nos: 2337-3761. In some embodiments, the lasso peptide biosynthesis component of the fusion protein has an amino acid sequence that has greater than 90% sequence identity to any one of peptide Nos: 2337-3761. In some embodiments, the lasso peptide biosynthesis component of the fusion protein has an amino acid sequence that has greater than 95% sequence identity to any one of peptide Nos: 2337-3761. In some embodiments, the lasso peptide biosynthesis component of the fusion protein has an amino acid sequence that has greater than 99% sequence identity to any one of peptide Nos: 2337-3761.

In some embodiments, the lasso peptide biosynthesis component of the fusion protein comprises the sequences of an RRE or a functional variant thereof. Particularly, the lasso peptide biosynthesis component of the fusion protein has an amino acid sequence selected from peptide Nos: 3762-4593. In some embodiments, the lasso peptide biosynthesis component of the fusion protein has an amino acid sequence that has greater than 30% sequence identity to any one of peptide Nos: 3762-4593. In some embodiments, the lasso peptide biosynthesis component of the fusion protein has an amino acid sequence that has greater than 40% sequence identity to any one of peptide Nos: 3762-4593. In some embodiments, the lasso peptide biosynthesis component of the fusion protein has an amino acid sequence that has greater than 50% sequence identity to any one of peptide Nos: 3762-4593. In some embodiments, the lasso peptide biosynthesis component of the fusion protein has an amino acid sequence that has greater than 60% sequence identity to any one of peptide Nos: 3762-4593. In some embodiments, the lasso peptide biosynthesis component of the fusion protein has an amino acid sequence that has greater than 70% sequence identity to any one of peptide Nos: 3762-4593. In some embodiments, the lasso peptide biosynthesis component of the fusion protein has an amino acid sequence that has greater than 80% sequence identity to any one of peptide Nos: 3762-4593. In some embodiments, the lasso peptide biosynthesis component of the fusion protein has an amino acid sequence that has greater than 90% sequence identity to any one of peptide Nos: 3762-4593. In some embodiments, the lasso peptide biosynthesis component of the fusion protein has an amino acid sequence that has greater than 95% sequence identity to any one of peptide Nos: 3762-4593. In some embodiments, the lasso peptide biosynthesis component of the fusion protein has an amino acid sequence that has greater than 99% sequence identity to any one of peptide Nos: 3762-4593.

In some embodiments, the lasso peptide biosynthesis component of the fusion protein comprises the sequences of a lasso peptidase and an RRE. Particularly, in some embodiments, the lasso peptide biosynthesis component of the fusion protein comprises the sequences of a functional variant of the lasso peptidase and an RRE. In some embodiments, the lasso peptide biosynthesis component of the fusion protein comprises the sequences of a lasso peptidase and a functional variant of an RRE. In some embodiments, the lasso peptide biosynthesis component of the fusion protein comprises the sequences of a functional variant of the lasso peptidase and a functional variant of the RRE. Particularly, the lasso peptide biosynthesis component of the fusion protein has an amino acid sequence selected from peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greaterthan 30% sequence identity to any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 40% sequence identity to any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 50% sequence identity to any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 60% sequence identity to any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 70% sequence identity to any one ofpeptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 80% sequence identity to any one ofpeptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 90% sequence identity to any one ofpeptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greater than 95% sequence identity to any one ofpeptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562. In some embodiments, the lasso peptide biosynthesis component of the fusion protein has an amino acid sequence that has greaterthan 99% sequence identity to any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, or 4562.

In some embodiments, the lasso peptide biosynthesis component of the fusion protein comprises the sequences of a lasso cyclase and an RRE. Particularly, in some embodiments, the lasso peptide biosynthesis component of the fusion protein comprises the sequences of a functional variant of the lasso cyclase and an RRE. In some embodiments, the lasso peptide biosynthesis component of the fusion protein comprises the sequences of a lasso cyclase and a functional variant of an RRE. In some embodiments, the lasso peptide biosynthesis component of the fusion protein comprises the sequences of a functional variant of the lasso cyclase and a functional variant of the RRE. Particularly, the lasso peptide biosynthesis component of the fusion protein has an amino acid sequence selected from peptide NO: 2504 or 3608. In some embodiments, the lasso peptide biosynthesis component of the fusion protein has an amino acid sequence that has greater than 30% sequence identity to any one of peptide Nos: 2504 or 3608. In some embodiments, the lasso peptide biosynthesis component of the fusion protein has an amino acid sequence that has greater than 40% sequence identity to any one of peptide Nos: 2504 or 3608. In some embodiments, the lasso peptide biosynthesis component of the fusion protein has an amino acid sequence that has greater than 50% sequence identity to any one of peptide Nos: 2504 or 3608. In some embodiments, the lasso peptide biosynthesis component of the fusion protein has an amino acid sequence that has greater than 60% sequence identity to any one of peptide Nos: 2504 or 3608. In some embodiments, the lasso peptide biosynthesis component of the fusion protein has an amino acid sequence that has greater than 70% sequence identity to any one of peptide Nos: 2504 or 3608. In some embodiments, the lasso peptide biosynthesis component of the fusion protein has an amino acid sequence that has greater than 80% sequence identity to any one of peptide Nos: 2504 or 3608. In some embodiments, the lasso peptide biosynthesis component of the fusion protein has an amino acid sequence that has greater than 90% sequence identity to any one of peptide Nos: 2504 or 3608. In some embodiments, the lasso peptide biosynthesis component of the fusion protein has an amino acid sequence that has greater than 95% sequence identity to any one of peptide Nos: 2504 or 3608. In some embodiments, the lasso peptide biosynthesis component of the fusion protein has an amino acid sequence that has greater than 99% sequence identity to any one of peptide Nos: 2504 or 3608.

In some embodiments, the lasso peptide biosynthesis component of the fusion protein comprises the sequences of a lasso peptidase and a lasso cyclase. Particularly, in some embodiments, the lasso peptide biosynthesis component of the fusion protein comprises the sequences of a functional variant of the lasso peptidase and a lasso cyclase. In some embodiments, the lasso peptide biosynthesis component of the fusion protein comprises the sequences of a lasso peptidase and a functional variant of a lasso cyclase. In some embodiments, the lasso peptide biosynthesis component of the fusion protein comprises the sequences of a functional variant of the lasso peptidase and a functional variant of the lasso cyclase. Particularly, the lasso peptide biosynthesis component of the fusion protein has an amino acid of peptide NO: 2903. In some embodiments, the lasso peptide biosynthesis component of the fusion protein has an amino acid sequence that has greater than 30% sequence identity to peptide No: 2903. In some embodiments, the lasso peptide biosynthesis component of the fusion protein has an amino acid sequence that has greater than 40% sequence identity to peptide No: 2903. In some embodiments, the lasso peptide biosynthesis component of the fusion protein has an amino acid sequence that has greater than 50% sequence identity to peptide No: 2903. In some embodiments, the lasso peptide biosynthesis component of the fusion protein has an amino acid sequence that has greater than 60% sequence identity to peptide No: 2903. In some embodiments, the lasso peptide biosynthesis component of the fusion protein has an amino acid sequence that has greater than 70% sequence identity to peptide No: 2903. In some embodiments, the lasso peptide biosynthesis component of the fusion protein has an amino acid sequence that has greater than 80% sequence identity to peptide No: 2903. In some embodiments, the lasso peptide biosynthesis component of the fusion protein has an amino acid sequence that has greater than 90% sequence identity to peptide No: 2903. In some embodiments, the lasso peptide biosynthesis component of the fusion protein has an amino acid sequence that has greater than 95% sequence identity to peptide No: 2903. In some embodiments, the lasso peptide biosynthesis component of the fusion protein has an amino acid sequence that has greater than 99% sequence identity to peptide No: 2903.

In some embodiments, the lasso peptide biosynthesis component of the fusion protein comprises the sequences of a lasso peptidase, a lasso cyclase, and an RRE. In some embodiments, the lasso peptide biosynthesis component of the fusion protein comprises the sequences of a functional variant of a lasso peptidase, a lasso cyclase, and an RRE. In some embodiments, the lasso peptide biosynthesis component of the fusion protein comprises the sequences of a lasso peptidase, a functional variant of a lasso cyclase, and an RRE. In some embodiments, the lasso peptide biosynthesis component of the fusion protein comprises the sequences of a lasso peptidase, a lasso cyclase, and a functional variant of an RRE. In some embodiments, the lasso peptide biosynthesis component of the fusion protein comprises the sequences of a functional variant of a lasso peptidase, a functional variant of a lasso cyclase, and an RRE. In some embodiments, the lasso peptide biosynthesis component of the fusion protein comprises the sequences of a functional variant of a lasso peptidase, a lasso cyclase, and a functional variant of an RRE. In some embodiments, the lasso peptide biosynthesis component of the fusion protein comprises the sequences of a lasso peptidase, a functional variant of a lasso cyclase, and a functional variant of an RRE. In some embodiments, the lasso peptide biosynthesis component of the fusion protein comprises the sequences of a functional variant of a lasso peptidase, a functional variant of a lasso cyclase, and a functional variant of an RRE.

In some embodiments, at least two of the lasso peptide biosynthesis components are fused via a cleavable linker, which upon cleavage, sever the at least two lasso peptide biosynthesis components from each other.

In some embodiments, the fusion protein comprising at least one lasso peptide biosynthesis component fused to (i) a secretion signal, or (ii) a purification tag. In some embodiments, the secretion signal is a periplasmic secretion signal. In particular embodiments, the periplasmic signal is a periplasmic space targeting signal sequence derived from TorA, PelB, OmpA, pIII, PhoA, DsbA, TolB, TorT, a substrate of the Type II Secretion System (T2SS), or a functional variant thereof. In particular embodiments, a fusion protein comprising at least one lasso peptide biosynthesis component and a periplasmic secretion signal is transported into the periplasmic space of a cell containing the fusion protein. In other embodiments, the secretion signal is an extracellular secretion signal. In particular embodiment, the extracellular signal is an extracellular space-targeting signal sequence derived from HlyA, a substrate of the Type 1 Secretion System (T1SS), or a functional variant thereof. In particular embodiments, a fusion protein comprising at least one lasso peptide biosynthesis component and an extracellular secretion signal is transported outside a cell containing the fusion protein. In some embodiments, the secretion signal is located at the N terminal end of the fusion protein. In other embodiments, the secretion signal is located at the C terminal end of the fusion protein.

In various embodiments, the fusion protein comprising at least one lasso peptide biosynthesis component fused to a purification tag. Any peptidic purification tag known in the art may be used in connection with the present disclosure, such as but not limited to, a His₆ tag, a FLAG tag, a streptavidin tag, etc. In some embodiments, fusion between the lasso peptide biosynthesis component and the purification tag is via a cleavable linker, which upon cleavage severs the biosynthesis component from the purification tag.

In some embodiments, the fusion protein comprising the lasso peptide biosynthesis component retains functionality of the lasso peptide biosynthesis. For example, a fusion protein comprising a lasso peptidase as provided herein is capable of processing a lasso precursor peptide into a lasso core peptide when contacted with the lasso precursor peptide under a suitable condition. For example, a fusion protein comprising a lasso cyclase as provided herein is capable of processing a lasso core peptide into a lasso peptide or a functional fragment of lasso peptide when contacted with the lasso core peptide under a suitable condition. For example, a fusion protein comprising a lasso peptidase and a lasso cyclase as provided herein is capable of processing a lasso precursor peptide into a lasso peptide or a functional fragment of lasso peptide when contacted with the lasso precursor peptide under a suitable condition. For example, a fusion protein comprising an RRE can function as a cofactor of a lasso peptidase or a lasso cyclase under a suitable condition.

In some embodiments, a fusion protein comprising at least one lasso peptide biosynthesis component is capable of processing a lasso precursor peptide into a lasso peptide or a functional fragment of lasso peptide in the periplasmic space of a cell comprising the fusion protein. In some embodiments, a fusion protein comprising at least one lasso peptide biosynthesis component is capable of processing a lasso core peptide into a lasso peptide or a functional fragment of lasso peptide in the periplasmic space of a cell comprising the fusion protein. In other embodiments, a fusion protein comprising at least one lasso peptide biosynthesis component is capable of processing a lasso precursor peptide displayed on a phage into a lasso peptide or a functional fragment of a lasso peptide. In other embodiments, a fusion protein comprising at least one lasso peptide biosynthesis component is capable of processing a lasso core peptide displayed on a phage into a lasso peptide or a functional fragment of a lasso peptide.

According to the present disclosure, the fusion protein described herein can be produced recombinantly. For example, one or more nucleic acid molecules encoding the fusion protein can be introduced into cells of a microbial strain that expresses the fusion protein. Particularly, in some embodiments, the expressed fusion protein can be isolated or purified using methods known in the art. In some embodiments, the microbial strain used to produce the fusion protein is a microbial organism known to be applicable to fermentation processes. Various microbial strains suitable for this purpose are known in the art, and some exemplary strains are Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Vibrio natriegens, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include yeast such as Saccharomyces cerevisiae.

In some embodiments, one or more fusion proteins as provided herein are expressed in a microbial cell, followed by the assembly into a phage. In some embodiments, the microbial cell is a host of the phage. In some embodiments, endogenous mechanism (e.g., endogenous proteins and/or cofactors) of the host cell enables the expression and assembly into a phage of the fusion protein. In other embodiments, exogenous mechanisms (e.g., exogenous genes) are introduced into the host cell to facilitate the expression and assembly into a phage of the fusion protein. In some embodiments, the host cell of the phage is also a microbial organism known to be applicable to fermentation processes as described herein. In some embodiments, the microbial cell is a bacterial cell or an archaeal cell. In some embodiments, the microbial cell is a natural host for the phage. Exemplary microbial organisms that can be used in connection with the present disclosure include but are not limited to Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Vibrio natriegens, Pseudomonas fluorescens, and Pseudomonas putida. E. coli is a particularly useful host organism since it is a well characterized microbial organism suitable for genetic engineering.

5.3.3 Nucleic Acids

In another aspect, provided herein are nucleic acid molecules encoding the fusion proteins as described herein and systems comprising one or more such nucleic acid molecules. Particularly, in some embodiments, systems comprising one or more nucleic acid molecules encoding the fusion proteins as described herein can be used to generate a phage display library of lasso peptides.

In some embodiments, provided herein is a nucleic acid molecule that encodes a fusion protein comprising a lasso peptide fragment. In some embodiments, the nucleic acid molecule encodes a fusion protein comprising the lasso peptide fragment fused to a phage coat protein. As described herein, the phage coat protein can be derived from a phage that assembles new phage particles in the periplasmic space of the host cell, such as an M13 phage, a fl phage or a fd phage, and phages that assembles new phage particles in the cytosol of the host cell, such as a T4 phage, a T7 phage, a λ (lambda) phage, an MS2 phage or a ΦX 174 phage. Particularly, in some embodiments, the phage coat protein is derived from p3, p6, p7, p8 or p9 of filamentous phages. In other embodiments, the phage coat protein is derived from SOC (small outer capsid) protein or HOC (highly antigenic outer capsid) protein of a T4 phage, pX of a T7 phage, pD or pV of a λ (lambda) phage, MS2 Coat Protein (CP) of an MS2 phage, or the Φ174 major spike protein G of a Φ174 phage.

In some embodiments, the nucleic acid molecule comprises a sequence encoding a phage coat protein, or a function variant thereof. In some embodiments, the functional variant of the phage coat protein has a different amino acid sequence as compared to the wild-type coat protein, but retain the functionality of the phage coat protein of assembly into the phage. In some embodiments, the sequence encoding the phage coat protein in the nucleic acid molecule contains one or more point mutations as compared to the wild-type sequence encoding the phage coat protein. In some embodiments, the sequence encoding the phage coat protein in the nucleic acid molecule comprises one or more deletion mutations as compared to the wild-type sequence encoding the phage coat protein. In some embodiments, the sequence encoding the phage coat protein in the second nucleic acid molecule comprises one or more insertion mutations as compared to the wild-type sequence encoding the phage coat protein. In some embodiments, the sequence encoding the phage coat protein in the nucleic acid molecule comprises one or more missense mutations as compared to the wild-type sequence encoding the phage coat protein. In some embodiments, the nucleic acid molecule comprises a truncated open reading frame that encodes a truncated version of the phage coat protein. In some embodiments, the truncation is at the 5′ end of the open reading frame. In other embodiments, the truncation is at the 3′ end of the open reading frame. In some embodiments, the nucleic acid encodes a domain shuffling mutant of the phage coat protein. In some embodiments, the second nucleic acid encodes a domain swapping mutant of the phage coat protein.

In some embodiments, the nucleic acid molecule further comprises a sequence encoding for a lasso peptide component. According to the present disclosure, the lasso peptide component can be (i) a lasso peptide; (ii) a functional fragment of a lasso peptide; (iii) a lasso precursor peptide, or (iv) a lasso core peptide. In some embodiments, the nucleic acid molecule comprises a sequence derived from Gene A of a lasso peptide biosynthesis gene cluster. Particularly, in some embodiments, the nucleic acid molecule comprises a sequence having the same sequence of a Gene A, or a fragment thereof. For example, in some embodiments, the fragment of Gene A comprised in the nucleic acid molecule is the open reading frame of Gene A. In other embodiments, the nucleic acid molecule comprises a variant of Gene A sequence, or a fragment thereof. For example, one or more mutations can be introduced into the Gene A sequence, or into a fragment of the Gene A sequence. In some embodiments, a variant of the Gene A sequence or a fragment of Gene A sequence (e.g. the ORF) has greater than 30% sequence identity to the Gene A sequence or the fragment of Gene A sequence (e.g., the ORF). The mutations can be introduced using various methods as described herein or known in the art.

Particularly, in some embodiments, the nucleic acid molecule comprises a sequence selected from any one of the odd numbers of SEQ ID NOS:1-2630. In some embodiments, the nucleic acid molecule comprises a sequence that has greater than 30% sequence identity to any one of the odd numbers of SEQ ID NOS:1-2630. In some embodiments, the nucleic acid molecule comprises a sequence that has greater than 40% sequence identity to any one of the odd numbers of SEQ ID NOS:1-2630. In some embodiments, the nucleic acid molecule comprises a sequence that has greater than 50% sequence identity to any one of the odd numbers of SEQ ID NOS:1-2630. In some embodiments, the nucleic acid molecule comprises a sequence that has greater than 60% sequence identity to any one of the odd numbers of SEQ ID NOS:1-2630. In some embodiments, the nucleic acid molecule comprises a sequence that has greater than 70% sequence identity to any one of the odd numbers of SEQ ID NOS:1-2630. In some embodiments, the nucleic acid molecule comprises a sequence that has greater than 80% sequence identity to any one of the odd numbers of SEQ ID NOS:1-2630. In some embodiments, the nucleic acid molecule comprises a sequence that has greater than 90% sequence identity to any one of the odd numbers of SEQ ID NOS:1-2630. In some embodiments, the nucleic acid molecule comprises a sequence that has greater than 95% sequence identity to any one of the odd numbers of SEQ ID NOS:1-2630. In some embodiments, the nucleic acid molecule comprises a sequence that has greater than 99% sequence identity to any one of the odd numbers of SEQ ID NOS:1-2630.

In some embodiments, the nucleic acid molecule further comprises a sequence encoding a secretion signal peptide. As provided herein, in some embodiments, the secretion signal peptide is a periplasmic secretion signal. In other embodiments, the secretion signal peptide is an extracellular secretion signal. In some embodiments, the sequence encoding the secretion signal peptide is located upstream to the sequences encoding the coat protein and the lasso peptide component. In some embodiments, the sequence encoding the secretion signal peptide is located downstream to the sequences encoding the coat protein and the lasso peptide component.

In some embodiments, the nucleic acid molecule further comprises one or more sequence encoding for a peptidic linker sequence. In some embodiments, the peptidic linker sequence is located between the lasso peptide fragment and the phage coat protein. In some embodiments, the peptidic linker sequence is located between the secretion signal peptide and the lasso peptide component. In some embodiments, the peptidic linker sequence is located between the secretion signal and the phage coat protein. In some embodiments, the peptidic linker is a cleavable linker. In some embodiments, the peptidic linker comprises cleavage site recognized and cleaved by a protease.

In some embodiments, the sequences encoding different components of the fusion protein are fused in frame with one another to code for a fusion protein comprising the different components. In some embodiments, the sequences coding for different components of the fusion protein are operably linked to the same expression regulatory element. In some embodiments, the sequences coding for different components of the fusion protein are operably linked to at least two different expression regulatory elements. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence.

In some embodiments, the nucleic acid molecule encoding the fusion protein comprising a lasso peptide component further comprises a replication origin sequence, such that the nucleic acid molecule can be replicated inside a cell. In some embodiments, the nucleic acid molecule encoding the fusion protein comprising a lasso peptide component further comprises a packaging signal sequence that enables packaging of the nucleic acid molecule into a phage. Various packaging signal sequences in genomes of phages can be used in connection with the present disclosure, such as those described in Fujisawa et al. Genes to Cells (1997) 2, 537-545. Various packaging signal sequences in genomes of other viruses can also be used in connection with the present disclosure, such as those described in Sun et al., Curr. Opin. Struct. Biol. 2010 February; 20(1): 114-120. In some embodiments, the replication origin sequence also serves as the packaging signal, such as the replication origin sequence of the fl phage. In some embodiments, the nucleic acid molecule encoding the fusion protein comprising a lasso peptide component is part of a cloning vector. In particular embodiments, the nucleic acid molecule encoding the fusion protein comprising a lasso peptide component is part of a plasmid. In particular embodiments, the nucleic acid molecule encoding the fusion protein comprising a lasso peptide component is part of a phagemid.

In particular embodiments, the nucleic acid molecule encoding the fusion protein is part of a phage genome. In some embodiments, the nucleic acid molecule encoding the fusion protein is configured to undergo homologous recombination to insert the coding sequence for the fusion protein into a phage genome sequence.

In some embodiments, provided herein is a nucleic acid molecule that encodes a fusion protein comprising a lasso peptide biosynthesis component. In some embodiments, the nucleic acid molecule encodes a fusion protein comprising the lasso peptide biosynthesis component fused to a (i) secretion signal, or (ii) a purification tag. The secretion signal or purification tag can be any secretion signal or purification tag described herein. In some embodiments, the lasso peptide biosynthesis component comprises one or more of a lasso peptidase, a lasso cyclase and an RRE.

In some embodiments, the nucleic acid comprises one or more sequence(s) derived from one or more gene(s) of a lasso peptide biosynthesis gene cluster. Particularly, in some embodiments, the nucleic acid comprises a sequence derived from Gene B of a lasso peptide biosynthesis gene cluster. In some embodiments, the nucleic acid comprises a sequence derived from Gene C of a lasso peptide biosynthesis gene cluster. In some embodiments, the nucleic acid comprises a sequence derived from Gene B and a sequence derived from Gene C of a lasso peptide biosynthesis gene cluster. In some embodiments, the nucleic acid comprises a sequence derived from a lasso peptide biosynthesis gene cluster that encodes an RRE. In some embodiments, the nucleic acid comprises a sequence derived from Gene B and a sequence derived from a lasso peptide biosynthesis gene cluster that encodes an RRE. In some embodiments, the nucleic acid comprises a sequence derived from Gene C and a sequence derived from a lasso peptide biosynthesis gene cluster that encodes an RRE. In some embodiments, the nucleic acid comprises a sequence derived from Gene B, a sequence derived from Gene C, and a sequence derived from a lasso peptide biosynthesis gene cluster that encodes an RRE.

According to the present disclosure, the nucleic acid molecule encoding a fusion protein comprising a lasso peptide biosynthesis component may comprises a sequence that is the same as a sequence of the lasso peptide biosynthesis gene cluster. Alternatively, the nucleic acid molecule encoding a fusion protein comprising a lasso peptide biosynthesis component may comprise a sequence that is a variant of a sequence of the lasso peptide biosynthesis gene cluster. In some embodiments, a variant of a sequence of the lasso peptide biosynthesis gene cluster has a different nucleic acid sequence as compared to the wild-type gene sequence, but still encodes a functional protein product of the lasso peptide biosynthesis gene cluster. In some embodiments, a nucleic acid variant has greater than 30% sequence identity to the wild-type gene sequence.

Particularly, in some embodiments, the nucleic acid molecule encoding a fusion protein comprising a lasso peptide biosynthesis component comprises a sequence encoding a lasso peptidase.

Particularly, in some embodiments, the nucleic acid molecule encoding a fusion protein comprising a lasso peptide biosynthesis component comprises a sequence encoding a lasso peptidase and a sequence encoding a lasso cyclase. In some embodiments, the nucleic acid molecule encoding a fusion protein comprising a lasso peptide biosynthesis component comprises a sequence encoding a lasso peptidase and a sequence encoding an RRE. In some embodiments, the nucleic acid molecule encoding a fusion protein comprising a lasso peptide biosynthesis component comprises a sequence encoding a lasso cyclase and a sequence encoding an RRE. In some embodiments, the nucleic acid molecule encoding a fusion protein comprising a lasso peptide biosynthesis component comprises a sequence encoding a lasso peptidase, a sequence encoding a lasso cyclase, and a sequence encoding an RRE.

Particularly, in some embodiment, the nucleic acid molecule encodes a fusion protein comprising a lasso peptidase and a lasso cyclase. In some embodiment, the nucleic acid molecule encodes a fusion protein comprising a lasso peptidase and an RRE. In some embodiment, the nucleic acid molecule encodes a fusion protein comprising a lasso cyclase and an RRE. In some embodiment, the nucleic acid molecule encodes a fusion protein comprising a lasso peptidase, a lasso cyclase, and an RRE. In these embodiments, the nucleic acid sequences encoding the two or more lasso peptide biosynthesis components can be any of the corresponding coding sequences disclosed herein.

Alternatively, in some embodiments, the nucleic acid molecule encodes one or more fusion proteins each comprises a lasso peptide biosynthesis component. Particularly, in some embodiments, the nucleic acid molecule encodes two fusion proteins, and one fusion protein comprises a lasso peptidase, and the other fusion protein comprises a lasso cyclase. Particularly, in some embodiments, the nucleic acid molecule encodes two fusion proteins, and one fusion protein comprises a lasso peptidase, and the other fusion protein comprises an RRE. Particularly, in some embodiments, the nucleic acid molecule encodes two fusion proteins, and one fusion protein comprises a lasso cyclase, and the other fusion protein comprises an RRE. Particularly, in some embodiments, the nucleic acid molecule encodes three fusion proteins, and the first fusion protein comprises a lasso peptidase, the second fusion protein comprises a lasso cyclase, and the third fusion protein comprises an RRE. In these embodiments, the nucleic acid sequences encoding the two or more lasso peptide biosynthesis components can be any of the corresponding coding sequences disclosed herein.

In some embodiments, the nucleic acid molecule further comprises a sequence encoding a secretion signal peptide. As provided herein, in some embodiments, the secretion signal peptide is a periplasmic secretion signal. In other embodiments, the secretion signal peptide is an extracellular secretion signal. In some embodiments, the sequence encoding the secretion signal peptide is located upstream to the sequences encoding the lasso peptide biosynthesis component. In some embodiments, the sequence encoding the secretion signal peptide is located downstream to the sequences encoding the lasso peptide biosynthesis component.

In some embodiments, the nucleic acid molecule further comprises one or more sequence encoding for a peptidic linker sequence. In some embodiments, the peptidic linker sequence is located between the lasso peptide biosynthesis component and the secretion signal peptide. In some embodiments, the peptidic linker sequence is located between two or more of lasso peptide biosynthesis components comprised with the fusion protein. In some embodiments, the peptidic linker is a cleavable linker. In some embodiments, the peptidic linker comprises cleavage site recognized and cleaved by a protease.

In some embodiments, the sequences encoding different components of the fusion protein and fused in flame with one another to code for a fusion protein comprising the different components (e.g., a fusion protein comprising a secretion signal peptide, a lasso peptidase and a lasso cyclase). In other embodiments, the sequences encoding different components of the fusion protein forms multiple open reading frames, each encoding a different protein or peptide. For example, in some embodiments, the nucleic acid molecule comprises three open reading flames, encoding a lasso peptidase, a lasso cyclase and an RRE, respectively. Particularly, in some embodiments, the nucleic acid molecule comprises three open reading frames, encoding a lasso peptidase fused to a secretion signal, a lasso cyclase fused to a secretion signal, and an RRE fused to a secretion signal, respectively. Particularly, in some embodiments, the nucleic acid molecule comprises three open reading frames, encoding a lasso peptidase fused to a purification tag, a lasso cyclase fused to a purification tag, and an RRE fused to a purification tag, respectively.

In some embodiments, the sequences coding for different components of the fusion protein are operably linked to the same expression regulatory element. In some embodiments, the sequences coding for different components of the fusion protein are operably linked to at least two different expression regulatory elements. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence.

In some embodiments, the nucleic acid molecule encoding the fusion protein comprising a lasso peptide biosynthesis component further comprises a replication origin sequence, such that the nucleic acid molecule can be replicated inside a cell. In some embodiments, the nucleic acid molecule encoding the fusion protein comprising a lasso peptide biosynthesis component is part of a cloning vector. In particular embodiments, the nucleic acid molecule encoding the fusion protein comprising a lasso peptide biosynthesis component is part of a plasmid.

In some embodiments, the nucleic acid sequences encoding the lasso peptide component and/or the lasso peptide biosynthesis component are derived from one or more naturally-existing lasso peptide biosynthetic gene clusters. In some embodiments, the coding sequences can be identified using the methods and systems described herein (e.g., in the section titled ‘Genomic Mining Tools for Genes coding Natural Lasso Peptides’). In some embodiments, a coding sequence can be mutated using methods described herein (e.g. in the section titled “Diversifying Lasso Peptides”).

5.3.4 Systems for Producing Phage Display Libraries

In one aspect, provided herein are also systems for producing phage display libraries of lasso peptides. In some embodiments, the system comprises one or more of the nucleic acid molecules provided herein. In some embodiments, the system further comprises components for expression of proteins encoded by the nucleic acid molecule. In some embodiments, the system further comprises components for assembling at least one of the expressed proteins into a phage displaying a lasso peptide component. In some embodiments, the system further comprises components for processing the lasso peptide component in the form of a lasso precursor peptide into a matured lasso peptide or functional fragment of lasso peptide. In some embodiments, the system further comprises components for processing the lasso peptide component in the form of a lasso core peptide into a matured lasso peptide or functional fragment of lasso peptide.

Particularly, in some embodiments, the system further comprises a cell. In some embodiments, the cell is capable of expressing one or more protein products encoded by the nucleic acid molecules of the system. In some embodiments, the cell is also capable of assembling one or more protein products encoded by the nucleic acid molecules of the system into a phage displaying a lasso peptide component. In some embodiments, the cell is also capable of processing a lasso peptide component in the form of a lasso precursor peptide into a matured lasso peptide or functional fragment of lasso peptide. In some embodiments, the cell is also capable of processing a lasso peptide component in the form of a lasso core peptide into a matured lasso peptide or functional fragment of lasso peptide.

In some embodiments, the system further comprises a cell-free biosynthesis system comprising a cell-free biosynthesis reaction mixture. In some embodiments, the cell-flee biosynthesis system is capable of expressing one or more protein products encoded by the nucleic acid molecules of the system. In some embodiments, the cell-free biosynthesis system is also capable of assembling one or more protein products encoded by the nucleic acid molecules of the system into a phage displaying a lasso peptide component. In some embodiments, the cell-free biosynthesis system is also capable of processing a lasso peptide component in the form of a lasso precursor peptide into a matured lasso peptide or functional fragment of lasso peptide. In some embodiments, the cell-free biosynthesis system is also capable of processing a lasso peptide component in the form of a lasso core peptide into a matured lasso peptide or functional fragment of lasso peptide.

5.3.4.1 Assembly of Lasso-Displaying Phage in the Periplasmic Space

In one aspect, provided herein are systems for producing a phage display library using a phage species that assembles progeny phage particles in the periplasmic space of a host cell (such as an M13 phage). Particularly, in some embodiments, the systems comprise (i) a first nucleic acid sequence encoding one or more structural proteins of a phage; (ii) a second nucleic acid sequence encoding at least one lasso peptide component; and (iii) a third nucleic acid sequence encoding at least one lasso peptide biosynthesis component.

Particularly, in some embodiments, the first nucleic acid sequence encodes one or more structural proteins of a phage. According to the present disclosure, the first nucleic acid sequence can be provided in the form of one or more vectors, such as plasmids. For example, in some embodiments, the first nucleic acid sequence is in the form of a plurality of different plasmids each encoding at least one structural protein of a phage. In some embodiments, the first nucleic is in the form of one plasmid encoding a plurality of phage structural proteins. Alternatively, in some embodiments, the first nucleic acid sequence is provided as a helper phage having the first nucleic acid sequence in the helper phage genome. In some embodiments, the helper phage genome lacks a packaging signal sequence that enables the packaging of the helper phage genome sequence into a phage. In some embodiments, the helper phage genome further comprises a sequence that prevents the packaging of the helper phage genome sequence into a phage. In some embodiments, the helper phage genome further comprises a sequence that reduces the efficiency of packaging the helper phage genome sequence into a phage. In particular embodiments, the helper phage is M13KO7. In particular embodiments, the helper phage is VCSM13.

In some embodiments, the phage structural proteins encoded by the first nucleic acid sequence can form a phage capsid. Particularly, in some embodiments, the first nucleic acid sequence encodes one structural protein that is capable of forming a phage capsid composed of the structural protein. In other embodiments, the first nucleic acid sequence encodes multiple different structural proteins that are capable of forming a phage capsid composed of different structural proteins.

In some embodiments, the first nucleic acid sequences encode at least one structural protein of a phage that is capable of assembling into a phage capsid together with a phage coat protein. Particularly, in some embodiments, the phage coat protein is encoded by a nucleic acid molecule different from the nucleic acid molecule containing the first nucleic acid sequence. For example, in some embodiments, the phage coat protein is encoded by the second nucleic acid sequence as provided herein. In some embodiments, the at least one phage structural protein encoded by the first nucleic acid sequence and the phage coat protein encoded by the second nucleic acid sequence are proteins derived from the same phage species. In other embodiments, the at least one phage structural protein encoded by the first nucleic acid sequence and the phage coat protein encoded by the second nucleic acid sequence are proteins derived from the different phage species.

In some embodiments, the first nucleic acid sequence encodes one or more structural protein of a phage that is a tailed phage, a non-tailed phage, a polyhedral phage, a filamentous phage, or a pleomorphic phage. Particularly, in some embodiments, the first nucleic acid sequences encodes one or more structural protein of a phage that is an M13 phage, a fl phage or a fd phage. Particularly, in some embodiments, the first nucleic acid sequence encodes one or more of proteins p3, p6, p7, p8, p9 of the M13 phage. In some embodiments, the first nucleic acid sequence encodes proteins p3, p6, 157, p8, and p9 of the M13 phage.

In some embodiments, in the first nucleic acid sequence, the sequences coding for different components of the fusion protein are operably linked to the same expression regulatory element. In some embodiments, the sequences coding for different components of the fusion protein are operably linked to at least two different expression regulatory elements. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence.

In some embodiments, the first nucleic acid sequence encoding the fusion protein comprising a lasso peptide biosynthesis component further comprises a replication origin sequence, such that a nucleic acid molecule comprising the first nucleic acid sequence can be replicated inside a cell. In some embodiments, the first nucleic acid sequence encoding the fusion protein comprising a lasso peptide biosynthesis component is part of a cloning vector. In particular embodiments, the first nucleic acid sequence encoding the fusion protein comprising a lasso peptide biosynthesis component is part of a plasmid.

In some embodiments, the second nucleic acid sequence encodes a fusion protein comprising a lasso peptide component, a phage coat protein and a periplasmic secretion signal. According to the present disclosure, the lasso peptide component in the fusion protein encoded by the second nucleic acid sequence can be (i) a lasso peptide; (ii) a functional fragment of lasso peptide; (iii) a lasso precursor peptide; and (iv) a lasso core peptide. In particular embodiments, the lasso peptide component in the fusion protein encoded by the second nucleic acid sequence is a lasso precursor peptide.

Particularly, in some embodiments, the second nucleic acid sequence comprises a sequence derived from a lasso peptide biosynthesis gene cluster. In some embodiments, the second nucleic acid sequence comprises a sequence derived from Gene A of a lasso peptide biosynthesis gene cluster. Particularly, in some embodiments, the nucleic acid molecule comprises a sequence having the same sequence of a Gene A, or a fragment thereof. For example, in some embodiments, the fragment of Gene A comprised in the nucleic acid molecule is the open reading flame of Gene A. In other embodiments, the nucleic acid molecule comprises a variant of Gene A sequence, or a fragment thereof. For example, one or more mutations can be introduced into the Gene A sequence, or into a fragment of the Gene A sequence. In some embodiments, a variant of the Gene A sequence or a fragment of Gene A sequence (e.g. the ORF) has greater than 30% sequence identity to the Gene A sequence or the fragment of Gene A sequence (e.g., the ORF). The mutations can be introduced using various methods as described herein or known in the art.

Particularly, in some embodiments, the nucleic acid molecule comprises a sequence selected from any one of the odd numbers of SEQ ID NOS:1-2630. In some embodiments, the nucleic acid molecule comprises a sequence that has greater than 30% sequence identity to any one of the odd numbers of SEQ ID NOS:1-2630. In some embodiments, the nucleic acid molecule comprises a sequence that has greater than 40% sequence identity to any one of the odd numbers of SEQ ID NOS:1-2630. In some embodiments, the nucleic acid molecule comprises a sequence that has greater than 50% sequence identity to any one of the odd numbers of SEQ ID NOS:1-2630. In some embodiments, the nucleic acid molecule comprises a sequence that has greater than 60% sequence identity to any one of the odd numbers of SEQ ID NOS:1-2630. In some embodiments, the nucleic acid molecule comprises a sequence that has greater than 70% sequence identity to any one of the odd numbers of SEQ ID NOS:1-2630. In some embodiments, the nucleic acid molecule comprises a sequence that has greater than 80% sequence identity to any one of the odd numbers of SEQ ID NOS:1-2630. In some embodiments, the nucleic acid molecule comprises a sequence that has greater than 90% sequence identity to any one of the odd numbers of SEQ ID NOS:1-2630. In some embodiments, the nucleic acid molecule comprises a sequence that has greater than 95% sequence identity to any one of the odd numbers of SEQ ID NOS:1-2630. In some embodiments, the nucleic acid molecule comprises a sequence that has greater than 99% sequence identity to any one of the odd numbers of SEQ ID NOS:1-2630.

In some embodiments, the second nucleic acid sequence further comprises a sequence encoding a phage coat protein. In some embodiments, the phage coat protein in the fusion protein encoded by the second nucleic acid is a functional variant of a phage coat protein.

In some embodiments, the second nucleic acid molecule comprises a sequence encoding a phage coat protein, or a function variant thereof. In some embodiments, the functional variant of the phage coat protein has a different amino acid sequence as compared to the wild-type coat protein, but retain the functionality of the phage coat protein of assembly into the phage. In some embodiments, the sequence encoding the coat protein in the second nucleic acid molecule contains one or more point mutations as compared to the wild-type sequence encoding the phage coat protein. In some embodiments, the sequence encoding the phage coat protein in the second nucleic acid molecule comprises one or more deletion mutations as compared to the wild-type sequence encoding the phage coat protein. In some embodiments, the sequence encoding the phage coat protein in the second nucleic acid molecule comprises one or more insertion mutations as compared to the wild-type sequence encoding the phage coat protein. In some embodiments, the sequence encoding the phage coat protein in the second nucleic acid molecule comprises one or more missense mutations as compared to the wild-type sequence encoding the phage coat protein. In some embodiments, the second nucleic acid molecule comprises a truncated open reading frame that encodes a truncated version of the phage coat protein. In some embodiments, the truncation is at the 5′ end of the open reading frame. In other embodiments, the truncation is at the 3′ end of the open reading frame. In some embodiments, the second nucleic acid encodes a domain shuffling mutant of the phage coat protein. In some embodiments, the second nucleic acid encodes a domain swapping mutant of the phage coat protein.

In some embodiments, the second nucleic acid sequence further comprises a sequence encoding a periplasmic secretion signal. In some embodiments, the periplasmic secretion signal in the fusion protein encoded by the second nucleic acid sequence is a periplasmic space-targeting signal sequence derived from TorA, PelB, OmpA, pi, PhoA, DsbA, TolB, TorT, a substrate of the Type II Secretion System (T2SS), or a functional variant thereof.

According to the present disclosure, the different fragments of the second nucleic acid sequence can have various orientations with respect to one another. For example, in some embodiments, the sequence encoding for the lasso peptide component is located upstream to the sequence encoding the phage coat protein. In some embodiments, the sequence encoding for the lasso peptide component is located upstream to the sequence encoding the periplasmic secretion signal. In some embodiments, the sequence encoding the coat protein is located upstream to the sequence encoding the lasso peptide component. In some embodiments, the sequence encoding for the lasso peptide component is located upstream to the sequence encoding the periplasmic secretion signal. In some embodiments, the sequence encoding the periplasmic secretion signal is located upstream to the sequence encoding the lasso peptide component. In some embodiments, the sequence encoding the periplasmic secretion signal is located upstream to the sequence encoding the phage coat protein. In some embodiments, the sequence encoding the periplasmic secretion signal is located upstream of the sequence encoding the lasso peptide component, which in turn is upstream to the sequence encoding the phage coat protein.

In some embodiments, the second nucleic acid molecule further comprises one or more sequence encoding for a peptidic linker sequence. In some embodiments, the sequence encoding the peptidic linker sequence is located between the sequence encoding the lasso peptide fragment and the sequence encoding the phage coat protein. In some embodiments, the sequence encoding the peptidic linker sequence is located between the sequence encoding the secretion signal peptide and the sequence encoding the lasso peptide component. In some embodiments, the peptidic linker sequence is located between the sequence encoding the secretion signal and the sequence encoding the phage coat protein. In some embodiments, the peptidic linker is a cleavable linker. In some embodiments, the peptidic linker comprises cleavage site recognized and cleaved by a protease.

In some embodiments, in the second nucleic acid sequence, the different sequences encoding different components of the fusion protein are fused in frame with one another to code for the fusion protein comprising the different components. In some embodiments, the sequence encoding the fusion protein is operably linked to an expression regulatory element. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence.

In some embodiments, the second nucleic acid sequence encoding the fusion protein comprising a lasso peptide component further comprises a replication origin sequence, such that the nucleic acid molecule can be replicated inside a cell. In some embodiments, the second nucleic acid sequence encoding the fusion protein comprising a lasso peptide component further comprises a packaging signal sequence that enables packaging of a nucleic acid molecule comprising the second nucleic acid sequence into a phage. Various packaging signal sequences in genomes of phages can be used in connection with the present disclosure, such as those described in Fujisawa et al. Genes to Cells (1997) 2, 537-545; Supra. Various packaging signal sequences in genomes of other viruses can also be used in connection with the present disclosure, such as those described in Sun et al., Curr. Opin. Struct. Biol. 2010 February; 20(1): 114-120; Supra. In some embodiments, the replication origin sequence also serves as the packaging signal, such as the replication origin sequence of the fl phage. In some embodiments, the second nucleic acid sequence encoding the fusion protein comprising a lasso peptide component is part of a cloning vector. In particular embodiments, the second nucleic acid sequence encoding the fusion protein comprising a lasso peptide component is part of a plasmid. In particular embodiments, the second nucleic acid sequence encoding the fusion protein comprising a lasso peptide component is part of a phagemid.

In some embodiments, the third nucleic acid sequence encodes one or more fusion protein each comprising at least one lasso peptide biosynthesis component. In some embodiments, the third nucleic acid sequence encodes one or more fusion protein each comprising a lasso peptide biosynthesis component fused to a (i) secretion signal, or (ii) a purification tag. In various embodiments, the secretion signal or purification tag can be any secretion signal or purification tag described herein. In some embodiments, the lasso peptide biosynthesis component of the fusion protein encoded by the third nucleic acid sequence comprises one or more of a lasso peptidase, a lasso cyclase and an RRE.

In some embodiments, the third nucleic acid sequence comprises one or more sequence(s) derived from one or more gene(s) of a lasso peptide biosynthesis gene cluster. Particularly, in some embodiments, the third nucleic acid sequence comprises a sequence derived from Gene B of a lasso peptide biosynthesis gene cluster. In some embodiments, the third nucleic acid sequence comprises a sequence derived from Gene C of a lasso peptide biosynthesis gene cluster. In some embodiments, the third nucleic acid sequence comprises a sequence derived from Gene B and a sequence derived from Gene C of a lasso peptide biosynthesis gene cluster. In some embodiments, the third nucleic acid sequence comprises a sequence derived from a lasso peptide biosynthesis gene cluster that encodes an RRE. In some embodiments, the third nucleic acid sequence comprises a sequence derived from Gene B and a sequence derived from a lasso peptide biosynthesis gene cluster that encodes an RRE. In some embodiments, the third nucleic acid sequence comprises a sequence derived from Gene C and a sequence derived from a lasso peptide biosynthesis gene cluster that encodes an RRE. In some embodiments, the third nucleic acid sequence comprises a sequence derived from Gene B, a sequence derived from Gene C, and a sequence derived from a lasso peptide biosynthesis gene cluster that encodes an RRE.

According to the present disclosure, in some embodiments, the third nucleic acid sequence encoding a fusion protein comprising a lasso peptide biosynthesis component may comprises a sequence that is the same as a sequence of the lasso peptide biosynthesis gene cluster. Alternatively, the third nucleic acid sequence encoding a fusion protein comprising a lasso peptide biosynthesis component may comprise a sequence that is a variant of a sequence of the lasso peptide biosynthesis gene cluster. In some embodiments, a variant of a sequence of the lasso peptide biosynthesis gene cluster has a different nucleic acid sequence as compared to the wild-type gene sequence, but still encodes a functional protein product of the lasso peptide biosynthesis gene cluster. In some embodiments, a nucleic acid variant has greater than 30% sequence identity to the wild-type gene sequence.

Particularly, in some embodiments, the third nucleic acid sequence encoding a fusion protein comprising a lasso peptide biosynthesis component comprises a sequence encoding a lasso peptidase.

Particularly, in some embodiments, the third nucleic acid sequence encoding a fusion protein comprising a lasso peptide biosynthesis component comprises a sequence encoding a lasso cyclase.

Particularly, in some embodiments, the third nucleic acid sequence encoding a fusion protein comprising a lasso peptide biosynthesis component comprises a sequence encoding an RRE.

Particularly, in some embodiments, the third nucleic acid sequence encoding a fusion protein comprising a lasso peptide biosynthesis component comprises a sequence encoding a lasso peptidase and a sequence encoding a lasso cyclase. In some embodiments, the third nucleic acid sequence encoding a fusion protein comprising a lasso peptide biosynthesis component comprises a sequence encoding a lasso peptidase and a sequence encoding an RRE. In some embodiments, the third nucleic acid sequence encoding a fusion protein comprising a lasso peptide biosynthesis component comprises a sequence encoding a lasso cyclase and a sequence encoding an RRE. In some embodiments, the third nucleic acid sequence encoding a fusion protein comprising a lasso peptide biosynthesis component comprises a sequence encoding a lasso peptidase, a sequence encoding a lasso cyclase, and a sequence encoding an RRE.

In some embodiments, the third nucleic acid sequence further comprises a sequence encoding a secretion signal peptide. As provided herein, in some embodiments, the secretion signal peptide is a periplasmic secretion signal. In other embodiments, the secretion signal peptide is an extracellular secretion signal. In some embodiments, the sequence encoding the secretion signal peptide is located upstream to the sequences encoding the lasso peptide biosynthesis component. In some embodiments, the sequence encoding the secretion signal peptide is located downstream to the sequences encoding the lasso peptide biosynthesis component.

In some embodiments, the third nucleic acid sequence further comprises a sequence encoding a purification tag. The encoded purification tag can be any purification tag provided herein. In some embodiments, the sequence encoding the purification tag is located upstream to the sequences encoding the lasso peptide biosynthesis component. In some embodiments, the sequence encoding the purification tag is located downstream to the sequences encoding the lasso peptide biosynthesis component.

In some embodiments, the third nucleic acid sequence further comprises one or more sequence encoding for a peptidic linker sequence. In some embodiments, the peptidic linker sequence is located between the lasso peptide biosynthesis component and the secretion signal peptide. In some embodiments, the peptidic linker sequence is located between two or more of lasso peptide biosynthesis components comprised with the fusion protein. In some embodiments, the peptidic linker is a cleavable linker. In some embodiments, the peptidic linker comprises cleavage site recognized and cleaved by a protease.

In some embodiments, in the third nucleic acid sequence, the sequences encoding different components of the fusion protein and fused in flame with one another to code for a fusion protein comprising the different components (e.g., a fusion protein comprising a secretion signal peptide, a lasso peptidase and a lasso cyclase). In other embodiments, the sequences encoding different components of the fusion protein forms multiple open reading frames, each encoding a different protein or peptide. For example, in some embodiments, the third nucleic acid sequence comprises three open reading frames, encoding a lasso peptidase, a lasso cyclase and an RRE, respectively. Particularly, in some embodiments, the third nucleic acid sequence comprises three open reading flames, encoding a lasso peptidase fused to a secretion signal, a lasso cyclase fused to a secretion signal, and an RRE fused to a secretion signal, respectively. Particularly, in some embodiments, the nucleic acid molecule comprises three open reading flames, encoding a lasso peptidase fused to a purification tag, a lasso cyclase fused to a purification tag, and an RRE fused to a purification tag, respectively.

According to the present disclosure, the third nucleic acid sequence can be provided in the form of one or more vectors, such as plasmids. For example, in some embodiments, the third nucleic acid sequence is in the form of a plurality of different plasmids each encoding a fusion protein comprising at least one lasso peptide biosynthesis component. In some embodiments, the third nucleic is in the form of one plasmid encoding a plurality of fusion proteins each comprising a lasso peptide biosynthesis component.

In some embodiments, in the third nucleic acid sequence, the sequences coding for different components of the fusion protein are operably linked to the same expression regulatory element. In some embodiments, the sequences coding for different components of the fusion protein are operably linked to at least two different expression regulatory elements. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence.

In some embodiments, the third nucleic acid sequence encoding the fusion protein comprising a lasso peptide biosynthesis component further comprises a replication origin sequence, such that a nucleic acid molecule comprising the third nucleic acid sequence can be replicated inside a cell. In some embodiments, the third nucleic acid sequence encoding the fusion protein comprising a lasso peptide biosynthesis component is part of a cloning vector. In particular embodiments, the third nucleic acid sequence encoding the fusion protein comprising a lasso peptide biosynthesis component is part of a plasmid.

According to the present disclosure, in a system for producing a phage display library of lasso peptides, one or more of the first, second and third nucleic acid sequences can form part of the same nucleic acid molecule. Particularly, in some embodiments, the system comprises (i) a first nucleic acid molecule comprising any one of the first nucleic acid sequences as provided herein; (ii) a second nucleic acid molecule comprising any one of the second nucleic acid sequences as provided herein; and (iii) a third nucleic acid molecule comprising any one of the third nucleic acid sequences as provided herein. In some embodiments, the system comprises (i) a first nucleic acid molecule comprising any one of the first nucleic acid sequences and any one of the second nucleic acid sequences as provided herein; and (ii) a second nucleic acid molecule comprising any one of the third nucleic acid sequences as provided herein. In some embodiments, the system comprises (i) a first nucleic acid molecule comprising any one of the first nucleic acid sequences and any one of the third nucleic acid sequences as provided herein; and (ii) a second nucleic acid molecule comprising any one of the second nucleic acid sequences as provided herein. In some embodiments, the system comprises (i) a first nucleic acid molecule comprising any one of the second nucleic acid sequences and any one of the third nucleic acid sequences as provided herein; and (ii) a second nucleic acid molecule comprising any one of the first nucleic acid sequences as provided herein. In some embodiments, the system comprises a nucleic acid molecule comprising any one of the first nucleic acid sequences, any one of the second nucleic acid sequences as provided herein, and any one of the third nucleic acid sequences as provided herein.

Furthermore, as disclosed herein, in various embodiments, at least one of the nucleic acid molecule in the system is a cloning vector. In various embodiments, at least one of the nucleic molecule in the system is a phagemid. In various embodiments, at least one of the nucleic acid molecule in the system is provided as a phage having a genome comprising the nucleic acid molecule.

In some embodiments, the system for producing the phage display library further comprises a cell. In some embodiments, the cell comprises one or more of the first nucleic acid sequence, the second nucleic acid sequence and the third nucleic acid sequence. In some embodiments, the cell is susceptible to transfection by a vector comprising one or more of the first nucleic acid sequence, the second nucleic acid sequence and the third nucleic acid sequence. In some embodiments, the cell is a host for a phage having a genome comprising the one or more of the first nucleic acid sequence, the second nucleic acid sequence and the third nucleic acid sequence.

In some embodiments, the cell is capable of expressing proteins encoded by the nucleic acid molecules of the system. In some embodiments, the cell is capable of assembling the proteins encoded by the first nucleic acid sequence into a phage capsid. In some embodiments, the cell is capable of assembling a protein encoded by the second nucleic acid sequence into a phage capsid. In some embodiments, the cell is capable of packaging a nucleic acid molecule comprising the second nucleic acid sequence into the phage capsid. In some embodiments, the cell has a periplasmic space. Particularly, in some embodiments, the cell is capable of transporting a protein encoded by the second nucleic acid sequence into the periplasmic space. In some embodiments, the cell is capable of transporting a protein encoded by the third nucleic acid sequence into the periplasmic space. In some embodiments, the cell is capable of transporting a protein encoded by the third nucleic acid sequence to the outside of the cell. In some embodiments, the cell is capable of processing a lasso precursor peptide into a lasso peptide or functional fragment of lasso peptide in the periplasmic space. In some embodiments, the cell is capable of assembling a protein encoded by the second nucleic acid sequence into a phage capsid. In some embodiments, the cell can perform the functions disclosed herein via an endogenous mechanism (e.g., endogens protein or signal pathway). In other embodiments, exogenous mechanism (e.g., exogenous genes) can be introduced into the cell to confer the one or more cellular functions described herein that lead to the production of a phage displaying a lasso peptide component. In some embodiments, exogenous mechanism can be introduced into the cell to supplement or strengthen an existing endogenous mechanism that lead to the production of a phage displaying a lasso peptide component.

In some embodiments, the cell is a microbial organism known to be applicable to fermentation processes as described herein. In some embodiments, the microbial cell is a bacterial cell or an archaeal cell. In some embodiments, the microbial cell is a host for the phage from which the structural protein encoded by the first nucleic acid sequence is derived. In some embodiments, the microbial cell is a host for the phage from which the coat protein encoded by the second nucleic acid sequence is derived. In some embodiments, the microbial cell is a host of a helper phage having a genome comprising the first nucleic acid sequence. Exemplary microbial organisms that can be used in connection with the present disclosure include but are not limited to Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Vibrio natriegens, Pseudomonas fluorescens, and Pseudomonas putida. E. coli is a particularly useful host organism since it is a well characterized microbial organism suitable for genetic engineering.

In some embodiments, the system for producing the phage display library further comprises a culture medium suitable for the growth of a microbial cell containing one or more of the first nucleic acid sequence, the second nucleic acid sequence and the third nucleic acid sequence is in a culture medium. In some embodiments, the system for producing the phage display library further comprises a culture medium suitable for the expression of phage protein by a microbial cell containing one or more of the first nucleic acid sequence, the second nucleic acid sequence and the third nucleic acid sequence is in a culture medium. In some embodiments, the system for producing the phage display library further comprises a culture medium suitable for the production of a phage by a microbial cell containing one or more of the first nucleic acid sequence, the second nucleic acid sequence and the third nucleic acid sequence is in a culture medium. In some embodiments, the culture medium comprises natural amino acid molecules. In some embodiments, the culture medium comprises non-natural amino acid molecules. In some embodiments, the culture medium comprises unusual amino acid molecules.

In some embodiments, one or more components of the system is purified. Particularly, in some embodiments, the system comprises one or more purified nucleic acid molecules comprising one or more of the first nucleic acid sequence, the second nucleic acid sequence and the third nucleic acid sequence. In some embodiments, the system comprises one or more purified proteins or peptide encoded by the first nucleic acid sequence, the second nucleic acid sequence or the third nucleic acid sequence. In particular embodiments, the system comprises purified fusion protein comprising one or more lasso peptide biosynthesis component. For example, in some embodiments, the system comprises a purified fusion protein comprising a lasso peptidase fused to a purification tag.

In particular embodiments, provided herein is a system comprising (i) one or more plasmid comprising any of the first nucleic acid sequence as described herein; (ii) a phagemid comprising any of the second nucleic acid sequences as described herein; and (iii) one or more plasmid comprising any of the third nucleic acid sequences as described herein.

In particular embodiments, provided herein is a system comprising (i) a helper phage comprising any of the first nucleic acid sequence as described herein; (ii) a phagemid comprising any of the second nucleic acid sequences as described herein; (iii) one or more plasmid comprising any of the third nucleic acid sequences as described herein; and (iv) a host cell of the helper phage.

In particular embodiments, provided herein is a system comprising (i) one or more plasmid comprising any of the first nucleic acid sequence as described herein; (ii) a phagemid comprising any of the second nucleic acid sequences as described herein; and (iii) one or more purified lasso peptide biosynthesis components.

In particular embodiments, provided herein is a system comprising (i) a helper phage comprising any of the first nucleic acid sequence as described herein; (ii) a phagemid comprising any of the second nucleic acid sequences as described herein; (iii) a host cell of the helper phage; and (iv) one or more purified lasso peptide biosynthesis components.

5.3.4.2 Assembly of Lasso-Displaying Phage in the Cytoplasm

In another aspect, provided herein are systems for producing a phage display library using a phage species that assembles progeny phage particles in the cytoplasm space of a host cell (such as a T4 phage). Particularly, in some embodiments, the systems comprise (i) a first nucleic acid sequence encoding one or more structural proteins of a bacteriophage; (ii) a second nucleic acid sequence encoding a first fusion protein comprising a lasso peptide component fused to a first coat protein of the bacteriophage; and (iii) a third nucleic acid sequence encoding at least one lasso peptide biosynthesis component.

Particularly, in some embodiments, the first nucleic acid sequence encodes one or more structural proteins of a phage. In some embodiments, the one or more structural proteins of the phage encoded by the first nucleic acid sequence include one or more coat proteins selected for displaying a peptide or protein on the phage capsid. In alternative embodiments, the first nucleic acid does not encode the one or more coat protein selected for displaying a peptide or protein on the phage capsid. In various embodiments, the displayed peptide or protein can be a lasso peptide component or a non-lasso peptide or protein.

According to the present disclosure, the first nucleic acid sequence can be provided in the form of a phage genome. In some embodiments, the phage genome is wild-type. In other embodiments, the phage genome is mutated. Particularly, in some embodiments, the mutated phage genome contains one or more null mutations in at least one endogenous sequence encoding the coat protein selected for displaying a peptide or protein on the phage capsid, such that the mutated phage genome can no longer produce the wild-type coat protein. In particular embodiments, the null mutation is made by deleting the endogenous sequence encoding the coat protein from the phage genome. In some embodiments, the coat protein is a nonessential outer capsid protein, such that null mutations to their respective coding sequences do not affect the viability, reproduction or infectivity of the phage. In various embodiments, the displayed peptide or protein can be a lasso peptide component or a non-lasso peptide or protein.

In some embodiments, the second nucleic acid sequence encodes for at least one fusion protein comprising the displayed peptide or protein fused to the selected phage coat protein. In particular embodiments, the second nucleic acid sequence encodes for a fusion protein comprising a lasso peptide component fused to a first phage coat protein. In some embodiments, the second nucleic acid sequence further encodes for a fusion protein comprising a non-lasso peptide or protein fused to a second phage coat protein. According to the present disclosure, the phage coat protein in the first and second fusion proteins can be the same coat protein or different coat proteins of the phage.

In some embodiments, the first and second nucleic acid sequences are in the same nucleic acid molecule. In other embodiments, the first and second nucleic acid sequence are in different nucleic acid molecules. In particular embodiments, the different nucleic acid molecules are configured to undergo homologous recombination to produce a recombinant molecule comprising both the first and second nucleic acid sequences. In some embodiments, the system further comprises enzymes catalyzing the recombination. In some embodiments, the enzymes catalyzing the recombination is provided in a host cell. In some embodiments, the enzyme catalyzing the recombination is provided in a cell-free biosynthesis reaction mixture.

Accordingly, in some embodiments, the present system comprises a mutated phage genome wherein the mutated genome comprises the first nucleic acid sequence encoding structural proteins of the phage. In some embodiments, the mutated phage genome further comprises the second nucleic acid sequence encoding for a first fusion protein comprising a lasso peptide component fused to a first coat protein. In some embodiments, the second nucleic acid sequence in the mutated phage genome further comprises a second fusion protein comprising a non-lasso peptide or protein fused to a second coat protein. In various embodiments, the first and second fusion proteins can be the same or different.

In some embodiments, the mutated phage genome comprises a null mutation in the endogenous sequence encoding the first protein coat protein. In some embodiments, the mutated phage genome comprises a null mutation in the endogenous sequence encoding the second protein coat protein. In various embodiments, the null mutation is a deletion of the endogenous encoding sequence from the phage genome.

In alternative embodiments, the mutated genome comprises the endogenous sequence encoding the first and/or second coat protein. In some embodiments, the expression levels of the endogenous coat protein and the fusion protein comprising the coat protein are controlled such that the expressed proteins are assembled onto a phage capsid at a desirable ratio. Particularly, in some embodiments, the expression levels are controlled via the use of expression regulatory elements. Particularly, the endogenous sequence encoding the coat protein and the sequence encoding the fusion protein comprising the coat protein can be operably linked to the same or different expression regulatory elements. Suitable expression regulatory elements are within the common knowledge of the art, such as a cis-regulatory element (CRE) of a gene, a promoter sequence, an enhancer sequence or an attenuator sequence.

In various embodiments, the non-lasso peptide or protein in the second fusion protein is configured to identify and/or manipulate its displaying phage, and thus the lasso peptide component displayed on said phage. In some embodiments, the non-lasso peptide or protein in the second fusion protein is an identification peptide. In some embodiments, the identification peptide is a detectable probe. In other embodiments, the identification peptide is a purification tag.

In some embodiments, the lasso peptide component and the identification peptide to be displayed are fused to different coat proteins of the phage. Particularly, in some embodiments, the phage is a non-naturally occurring T4 phage, and the lasso peptide component is fused to HOC, and the identification peptide is fused to SOC. Particularly, in some embodiments, the phage is a non-naturally occurring T4 phage, and the lasso peptide component is fused to SOC, and the identification peptide is fused to HOC. In some embodiments, the phage is a non-naturally occurring λ (lambda) phage, and the lasso peptide component is fused to pV, and the identification peptide is fused to pD. In some embodiments, the phage is a non-naturally occurring λ (lambda) phage, and the lasso peptide component is fused to pD, and the identification peptide is fused to pV.

In some embodiments, the lasso peptide component and the identification peptide to be displayed are fused to the same coat protein of the phage. Particularly, in some embodiments, the phage is a non-naturally occurring T4 phage, and the lasso peptide component is fused to HOC, and the identification peptide is fused to HOC. In some embodiments, the phage is a non-naturally occurring T4 phage, and the lasso peptide component is fused to SOC, and the identification peptide is fused to SOC. In some embodiments, the phage is a non-naturally occurring T7 phage, and the lasso peptide component is fused to pX, and the identification peptide is fused to pX. In some embodiments, the phage is a non-naturally occurring λ (lambda) phage, and the lasso peptide component is fused to pD, and the identification peptide is fused to pD. In some embodiments, the phage is a non-naturally occurring λ (lambda) phage, and the lasso peptide component is fused to pV, and the identification peptide is fused to pV.

In some embodiments, the second nucleic acid sequence encodes a fusion protein comprising a lasso peptide component and a phage coat protein. According to the present disclosure, the lasso peptide component in the fusion protein encoded by the second nucleic acid sequence can be (i) a lasso peptide; (ii) a functional fragment of lasso peptide; (iii) a lasso precursor peptide; and (iv) a lasso core peptide. In particular embodiments, the lasso peptide component in the fusion protein encoded by the second nucleic acid sequence is a lasso precursor peptide.

Particularly, in some embodiments, the second nucleic acid sequence comprises a sequence derived from a lasso peptide biosynthesis gene cluster. In some embodiments, the second nucleic acid sequence comprises a sequence derived from Gene A of a lasso peptide biosynthesis gene cluster. Particularly, in some embodiments, the nucleic acid molecule comprises a sequence having the same sequence of a Gene A, or a fragment thereof. For example, in some embodiments, the fragment of Gene A comprised in the nucleic acid molecule is the open reading frame of Gene A. In other embodiments, the nucleic acid molecule comprises a variant of Gene A sequence, or a fragment thereof. For example, one or more mutations can be introduced into the Gene A sequence, or into a fragment of the Gene A sequence. In some embodiments, a variant of the Gene A sequence or a fragment of Gene A sequence (e.g. the ORF) has greater than 30% sequence identity to the Gene A sequence or the fragment of Gene A sequence (e.g., the ORF). The mutations can be introduced using various methods as described herein or known in the art.

Particularly, in some embodiments, the nucleic acid molecule comprises a sequence selected from any one of the odd numbers of SEQ ID NOS:1-2630. In some embodiments, the nucleic acid molecule comprises a sequence that has greater than 30% sequence identity to any one of the odd numbers of SEQ ID NOS:1-2630. In some embodiments, the nucleic acid molecule comprises a sequence that has greater than 40% sequence identity to any one of the odd numbers of SEQ ID NOS:1-2630. In some embodiments, the nucleic acid molecule comprises a sequence that has greater than 50% sequence identity to any one of the odd numbers of SEQ ID NOS:1-2630. In some embodiments, the nucleic acid molecule comprises a sequence that has greater than 60% sequence identity to any one of the odd numbers of SEQ ID NOS:1-2630. In some embodiments, the nucleic acid molecule comprises a sequence that has greater than 70% sequence identity to any one of the odd numbers of SEQ ID NOS:1-2630. In some embodiments, the nucleic acid molecule comprises a sequence that has greater than 80% sequence identity to any one of the odd numbers of SEQ ID NOS:1-26308. In some embodiments, the nucleic acid molecule comprises a sequence that has greater than 90% sequence identity to any one of the odd numbers of SEQ ID NOS:1-2630. In some embodiments, the nucleic acid molecule comprises a sequence that has greater than 95% sequence identity to any one of the odd numbers of SEQ ID NOS:1-2630. In some embodiments, the nucleic acid molecule comprises a sequence that has greater than 99% sequence identity to any one of the odd numbers of SEQ ID NOS:1-2630.

In some embodiments, the second nucleic acid sequence further comprises a sequence encoding a phage coat protein. As described herein, the phage coat protein in the fusion protein encoded by the second nucleic acid can be derived from a T4 page, a T7 phage, a λ phage, an MS2 phage, or a ΦX174 phage. More particularly, in some embodiments, the phage coat protein in the fusion protein encoded by the second nucleic acid is derived from the SOC (small outer capsid) protein or HOC (highly antigenic outer capsid) protein of a T4 phage, pX of a T7 phage, pD or pV of a λ (lambda) phage, the MS2 Coat Protein (CP) of an MS2 phage, or the ΦX 174 major spike protein G of a ΦX 174 phage. In some embodiments, the phage coat protein in the fusion protein encoded by the second nucleic acid is a functional variant of a phage coat protein.

In some embodiments, the second nucleic acid molecule comprises a sequence encoding a phage coat protein, or a function variant thereof. In some embodiments, the functional variant of the phage coat protein has a different amino acid sequence as compared to the wild-type coat protein, but retain the functionality of the phage coat protein of assembly into the phage. In some embodiments, the sequence encoding the coat protein in the second nucleic acid molecule contains one or more point mutations as compared to the wild-type sequence encoding the phage coat protein. In some embodiments, the sequence encoding the phage coat protein in the second nucleic acid molecule comprises one or more deletion mutations as compared to the wild-type sequence encoding the phage coat protein. In some embodiments, the sequence encoding the phage coat protein in the second nucleic acid molecule comprises one or more insertion mutations as compared to the wild-type sequence encoding the phage coat protein. In some embodiments, the sequence encoding the phage coat protein in the second nucleic acid molecule comprises one or more missense mutations as compared to the wild-type sequence encoding the phage coat protein. In some embodiments, the second nucleic acid molecule comprises a truncated open reading frame that encodes a truncated version of the phage coat protein. In some embodiments, the truncation is at the 5′ end of the open reading frame. In other embodiments, the truncation is at the 3′ end of the open reading frame. In some embodiments, the second nucleic acid encodes a domain shuffling mutant of the phage coat protein. In some embodiments, the second nucleic acid encodes a domain swapping mutant of the phage coat protein.

According to the present disclosure, the different fragments of the second nucleic acid sequence can have various orientations with respect to one another. For example, in some embodiments, the sequence encoding for the lasso peptide component is located upstream to the sequence encoding the phage coat protein. In some embodiments, the sequence encoding the coat protein is located upstream to the sequence encoding the lasso peptide component.

In some embodiments, the second nucleic acid molecule further comprises one or more sequence encoding for a peptidic linker sequence. In some embodiments, the sequence encoding the peptidic linker sequence is located between the sequence encoding the lasso peptide fragment and the sequence encoding the phage coat protein. In some embodiments, the peptidic linker is a cleavable linker. In some embodiments, the peptidic linker comprises cleavage site recognized and cleaved by a protease.

In some embodiments, in the second nucleic acid sequence, the different sequences encoding different components of the fusion protein are fused in frame with one another to code for the fusion protein comprising the different components. In some embodiments, the sequence encoding the fusion protein is operably linked to an expression regulatory element. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence. In some embodiments, the second nucleic acid sequence encoding the fusion protein comprising a lasso peptide component further comprises a replication origin sequence, such that the nucleic acid molecule can be replicated inside a cell.

In some embodiments, the third nucleic acid sequence encodes one or more lasso peptide biosynthesis component. In some embodiments, the third nucleic acid sequence encodes one or more fusion protein each comprising a lasso peptide biosynthesis component fused to a purification tag. In various embodiments, the purification tag can be any purification tag described herein. In some embodiments, the lasso peptide biosynthesis component of the fusion protein encoded by the third nucleic acid sequence comprises one or more of a lasso peptidase, a lasso cyclase and an RRE.

In some embodiments, the third nucleic acid sequence comprises one or more sequence(s) derived from one or more gene(s) of a lasso peptide biosynthesis gene cluster. Particularly, in some embodiments, the third nucleic acid sequence comprises a sequence derived from Gene B of a lasso peptide biosynthesis gene cluster. In some embodiments, the third nucleic acid sequence comprises a sequence derived from Gene C of a lasso peptide biosynthesis gene cluster. In some embodiments, the third nucleic acid sequence comprises a sequence derived from Gene B and a sequence derived from Gene C of a lasso peptide biosynthesis gene cluster. In some embodiments, the third nucleic acid sequence comprises a sequence derived from a lasso peptide biosynthesis gene cluster that encodes an RRE. In some embodiments, the third nucleic acid sequence comprises a sequence derived from Gene B and a sequence derived from a lasso peptide biosynthesis gene cluster that encodes an RRE. In some embodiments, the third nucleic acid sequence comprises a sequence derived from Gene C and a sequence derived from a lasso peptide biosynthesis gene cluster that encodes an RRE. In some embodiments, the third nucleic acid sequence comprises a sequence derived from Gene B, a sequence derived from Gene C, and a sequence derived from a lasso peptide biosynthesis gene cluster that encodes an RRE.

According to the present disclosure, in some embodiments, the third nucleic acid sequence encoding a lasso peptide biosynthesis component may comprises a sequence that is the same as a sequence of the lasso peptide biosynthesis gene cluster. Alternatively, the third nucleic acid sequence encoding a lasso peptide biosynthesis component may comprise a sequence that is a variant of a sequence of the lasso peptide biosynthesis gene cluster. In some embodiments, a variant of a sequence of the lasso peptide biosynthesis gene cluster has a different nucleic acid sequence as compared to the wild-type gene sequence, but still encodes a functional protein product of the lasso peptide biosynthesis gene cluster. In some embodiments, a nucleic acid variant has greater than 30% sequence identity to the wild-type gene sequence.

Particularly, in some embodiments, the third nucleic acid sequence encoding a lasso peptide biosynthesis component comprises a sequence encoding a lasso peptidase.

Particularly, in some embodiments, the third nucleic acid sequence encoding a lasso peptide biosynthesis component comprises a sequence encoding a lasso cyclase.

Particularly, in some embodiments, the third nucleic acid sequence encoding a lasso peptide biosynthesis component comprises a sequence encoding an RRE

Particularly, in some embodiments, the third nucleic acid sequence encoding a lasso peptide biosynthesis component comprises a sequence encoding a lasso peptidase and a sequence encoding a lasso cyclase. In some embodiments, the third nucleic acid sequence encoding a fusion protein comprising a lasso peptide biosynthesis component comprises a sequence encoding a lasso peptidase and a sequence encoding an RRE. In some embodiments, the third nucleic acid sequence encoding a fusion protein comprising a lasso peptide biosynthesis component comprises a sequence encoding a lasso cyclase and a sequence encoding an RRE. In some embodiments, the third nucleic acid sequence encoding a fusion protein comprising a lasso peptide biosynthesis component comprises a sequence encoding a lasso peptidase, a sequence encoding a lasso cyclase, and a sequence encoding an RRE.

In some embodiments, the third nucleic acid sequence further comprises a sequence encoding a purification tag. The encoded purification tag can be any purification tag provided herein. In some embodiments, the sequence encoding the purification tag is located upstream to the sequences encoding the lasso peptide biosynthesis component. In some embodiments, the sequence encoding the purification tag is located downstream to the sequences encoding the lasso peptide biosynthesis component.

In some embodiments, the third nucleic acid sequence further comprises one or more sequence encoding for a peptidic linker sequence. In some embodiments, the peptidic linker sequence is located between the lasso peptide biosynthesis component and the secretion signal peptide. In some embodiments, the peptidic linker sequence is located between two or more of lasso peptide biosynthesis components comprised with the fusion protein. In some embodiments, the peptidic linker is a cleavable linker. In some embodiments, the peptidic linker comprises cleavage site recognized and cleaved by a protease.

In some embodiments, in the third nucleic acid sequence, the sequences encoding different components of the fusion protein and fused in flame with one another to code for a fusion protein comprising the different components (e.g., a fusion protein comprising a lasso peptidase and a lasso cyclase). In other embodiments, the sequences encoding different components of the fusion protein forms multiple open reading frames, each encoding a different protein or peptide. For example, in some embodiments, the third nucleic acid sequence comprises three open reading flames, encoding a lasso peptidase, a lasso cyclase and an RRE, respectively. Particularly, in some embodiments, the third nucleic acid sequence comprises three open reading flames, encoding a lasso peptidase fused to a purification tag, a lasso cyclase fused to a purification tag, and an RRE fused to a purification tag, respectively.

According to the present disclosure, the third nucleic acid sequence can be provided in the form of one or more vectors, such as plasmids. For example, in some embodiments, the third nucleic acid sequence is in the form of a plurality of different plasmids each encoding at least one lasso peptide biosynthesis component. In some embodiments, the third nucleic is in the form of one plasmid encoding multiple lasso peptide biosynthesis components.

In some embodiments, in the third nucleic acid sequence, the sequences coding for different lasso peptide biosynthesis components are operably linked to the same expression regulatory element. In some embodiments, the sequences coding for different lasso peptide biosynthesis components are operably linked to at least two different expression regulatory elements. In some embodiments, the expression regulatory element is a cis-regulatory element (CRE) of a gene. In some embodiments, the expression regulatory element is a promoter sequence. In some embodiments, the expression regulator element is an enhancer sequence. In some embodiments, the expression regulator element is an attenuator sequence.

In some embodiments, the third nucleic acid sequence encoding a lasso peptide biosynthesis component further comprises a replication origin sequence, such that a nucleic acid molecule comprising the third nucleic acid sequence can be replicated inside a cell. In some embodiments, the third nucleic acid sequence encoding a lasso peptide biosynthesis component is part of a cloning vector. In particular embodiments, the third nucleic acid sequence encoding a lasso peptide biosynthesis component is part of a plasmid.

According to the present disclosure, in a system for producing a phage display library of lasso peptides, one or more of the first, second and third nucleic acid sequences can form part of the same nucleic acid molecule. In some embodiments, the nucleic acid molecule can be a wild-type or mutated phage genome. In some embodiments, the structural proteins encoded by the first sequence can assemble into a protein capsid. In some embodiments, the phage genome comprising one or more of the first, second and third nucleic acid sequences can be packaged into the protein capsid.

In some embodiments, the second nucleic acid sequence encodes at least one fusion protein. In some embodiments, the at least one fusion proteins comprises a first fusion protein comprising a lasso peptide component fused to a coat protein of the phage. In some embodiments, the at least one fusion proteins further comprises a second fusion protein comprising a non-lasso peptide or protein fused to a coat protein of the phage. In various embodiments, the coat proteins in the first and the second fusion proteins can be the same or different.

In some embodiments, the first and second nucleic acid sequences of the present system are in the same nucleic acid molecule. In other embodiments, the first and second nucleic acid sequences of the present system are in separate nucleic acid molecules. Particularly, in some embodiments, the molecules containing the first and second nucleic acid sequences are capable of undergoing homologous recombination to produce a recombinant sequence containing both the first and second nucleic acid sequence.

In some embodiments, the first and second nucleic acid sequence can be provided in the form of a phage genome. Particularly, in some embodiments

5.3.5 Phage Display Library Members

In one aspect, provided herein are phage display libraries comprising a plurality of lasso peptide components. According to the present disclosure, the lasso peptide component present in the phage display library can be (i) a lasso peptide, (ii) a functional fragment of lasso peptide, (iii) a lasso precursor peptide; or (iv) a lasso core peptide. In some embodiments, the lasso peptide component of the fusion protein can undergo transition under a suitable condition among the different forms (i), (ii), (iii) and (iv).

In some embodiments, the library comprises at least one phage comprising a coat protein comprising the lasso peptide component. Particularly, in some embodiments, the lasso peptide component is displayed on the surface of the phage capsid. In some embodiments, the phage further comprises a nucleic acid molecule encoding at least part of the lasso peptide component. In some embodiments, the phage capsid encloses the nucleic acid molecule encoding at least part of the lasso peptide component. In some embodiments, the nucleic acid molecule is a phagemid.

In some embodiments, the nucleic acid molecule comprises the phage genome sequences. In specific embodiments, the nucleic acid sequence comprises the wild-type phage genome. In specific embodiments, the nucleic acid sequence comprises a mutated version of the phage genome. For example, in some embodiments, the mutated phage genome does not encode one or more wild-type coat proteins that are selected to make the fusion proteins for displaying lasso peptide component and other non-lasso peptide or protein components. In some embodiments, the mutated genome has a null mutation is one or more endogenous sequences encoding such coat proteins. In particular embodiments, the null mutation is introduced by deleting the endogenous sequence from the phage genome. Furthermore, in some embodiments, the mutated phage genome further comprises an exogenous sequence encoding a fusion protein containing the coat protein.

In particular embodiments, the nucleic acid molecule encodes a fusion protein comprising the lasso peptide component and the phage coat protein. In particular embodiments, the nucleic acid encodes a fusion protein comprising the lasso peptide component, the phage coat protein and a periplasmic secretion signal. In particular embodiments, the nucleic acid encodes a fusion protein comprising an identification peptide and a phage coat protein. In some embodiments, one or more of the phage coat protein forming the fusion proteins described herein are nonessential outer capsid proteins of the phage.

In some embodiments, the nucleic acid molecule encodes (i) a fusion protein comprising the lasso peptide component and the phage coat protein; and (ii) one or more phage structural proteins. Particularly, the one or more phage structural proteins and the fusion protein are capable of assembling together into a phage capsid. In some embodiments, the nucleic acid molecule further comprises a packaging signal that is recognized by the one or more phage structural proteins and is packaged into the phage capsid. In some embodiments, the coat protein in the fusion protein and the one or more structural proteins are derived from the same phage species. In other embodiments, the coat protein in the fusion protein and the one or more structural proteins are derived from different phage species. Many phage species are known in the art and can be used in connection with the present disclosure. For example, the coat protein or the one or more structural protein may be derived from a phage that assembles new phage particles in the periplasmic space of the host cell, such as an M13 phage, a fl phage or a fd phage, and phages that assembles new phage particles in the cytosol of the host cell, such as a T4 phage, a T7 phage, a λ (lambda) phage, an MS2 phage or a ΦX714 phage.

Particularly, in some embodiments, the phage coat protein is derived from p3, p6, p7, p8 or p9 of filamentous phages. In other embodiments, the phage coat protein is derived from SOC (small outer capsid) protein or HOC (highly antigenic outer capsid) protein of a T4 phage, pX of a T7 phage, pD or pV of a λ (lambda) phage, the MS2 Coat Protein (CP) of an MS2 phage, or the ΦX174 major spike protein G of a ΦX174 phage.

In some embodiments, the nucleic acid encodes a phage protein (e.g., the coat protein portion of the fusion protein, or the structural protein) that is a functional variant of the wild-type phage protein. In some embodiments, the phage protein encoded by the nucleic acid has greater than 30% sequence identity to the wild-type phage protein. In some embodiments, the phage protein encoded by the nucleic acid has greater than 40% sequence identity to the wild-type phage protein. In some embodiments, the phage protein encoded by the nucleic acid has greater than 50% sequence identity to the wild-type phage protein. In some embodiments, the phage protein encoded by the nucleic acid has greater than 60% sequence identity to the wild-type phage protein. In some embodiments, the phage protein encoded by the nucleic acid has greater than 70% sequence identity to the wild-type phage protein. In some embodiments, the phage protein encoded by the nucleic acid has greater than 80% sequence identity to the wild-type phage protein. In some embodiments, the phage protein encoded by the nucleic acid has greater than 90% sequence identity to the wild-type phage protein. In some embodiments, the phage protein encoded by the nucleic acid has greater than 95% sequence identity to the wild-type phage protein. In some embodiments, the phage protein encoded by the nucleic acid has greater than 99% sequence identity to the wild-type phage protein. In particular embodiments, the phage protein encoded by the nucleic acid is a truncated version of the wild-type protein. In particular embodiments, the nucleic acid molecule comprises any one of the first nucleic acid sequences as described herein, and any one of the second nucleic acid sequences as described herein.

In some embodiments, the nucleic acid molecule encodes (i) a fusion protein comprising the lasso peptide component and the phage coat protein; (ii) one or more phage structural proteins; and (iii) at least one fusion protein each comprising one or more lasso peptide biosynthesis components. In some embodiments, the nucleic acid molecule comprises any one of the first nucleic acid sequences as described herein, any one of the second nucleic acid sequences as described herein, and any one of the third nucleic acid sequences as described herein.

In some embodiments, the phage displays a lasso peptide. In some embodiments, the phage displays a functional fragment of lasso peptide. In some embodiments, the phage displays a lasso precursor peptide. In some embodiments, the phage displays a lasso core peptide.

In some embodiments, the phage is in contact with one or more lasso peptide biosynthesis component. Particularly, in some embodiments, the phage is in contact with a lasso peptidase. Additionally or alternatively, in some embodiments, the phage is in contact with a lasso cyclase. Additionally or alternatively, in some embodiments, the phage is in contact with a REE. In some embodiments, the phage is in contact with a fusion protein comprising one or more lasso peptide biosynthesis component. In some embodiments, the phage is in contact with a fusion protein comprising a lasso peptidase and a lasso cyclase. In some embodiments, the phage is in contact with a fusion protein comprising a lasso peptidase and an RRE. In some embodiments, the phage is in contact with a fusion protein comprising a lasso cyclase and an RRE. In some embodiments, the phage is in contact with a fusion protein comprising a lasso peptidase, a lasso cyclase and an RRE. In some embodiments, the phage is in contact with any of the fusion proteins described herein. I some embodiments, the phage is in contact with any of the proteins encoded by the nucleic acid molecules described herein. In some embodiments, the phage is in contact with any of the proteins encoded by any of the third nucleic acid sequences described herein. In some embodiments, the phage is in contact with one or more lasso peptide biosynthesis components that are purified.

In particular embodiments, a phage displaying a lasso precursor peptide is in contact with a lasso peptidase and a lasso cyclase. In some embodiments, the phage is further in contact with an RRE. In some embodiments, the phage is contacted with the lasso peptide biosynthesis components under a suitable condition for the lasso peptide biosynthesis components to convert the lasso precursor peptide into a lasso peptide or a functional fragment of lasso peptide. In Particular embodiments, a phage displaying a lasso core peptide is in contact with a lasso cyclase. In some embodiments, the phage is further in contact with an RRE. In some embodiments, the phage is in contact with one or more lasso peptide biosynthesis components that are purified. In some embodiments, the phage is contacted with the lasso peptide biosynthesis components under a suitable condition for the lasso peptide biosynthesis components to convert the lasso core peptide into a lasso peptide or a functional fragment of lasso peptide. In some embodiments, the phage is in a culture medium of a host microbial organism. In some embodiments, the phage is purified. In some embodiments, the one or more lasso peptide biosynthesis components are purified.

In some embodiments, a phage displaying a lasso peptide component is produced by a host cell. In some embodiments, the host cell produces the phage in its periplasmic space. In other embodiments, the host cell produces the phage in its cytoplasm. In some embodiments, a phage displaying a lasso peptide component is produced in a cell-free biosynthesis reaction mixture as described herein.

In some embodiments, the phage display library comprises one member. In some embodiments, the phage display library comprises a plurality of different members. In some embodiments, each member of the library comprises a phage displaying a unique lasso peptide or functional fragment of lasso peptide. In some embodiments, each member of the library also comprises a unique identification mechanism for identifying or manipulation of the member. For example, in some embodiments, each member of the library is associated with a unique location on a solid support, and the locational information is used to identify the member associated therewith. In other embodiments, each member of the library comprises a phage displaying a unique lasso peptide component, and also displaying an identification peptide. Particularly, in some embodiments, the identification peptide is configured to produce a detectable signal for identification of the phage, and the unique lasso peptide component displayed thereon. In some embodiments, the identification peptide is configured to manipulate the phage and thus the unique lasso peptide component displayed thereon. In particular embodiments, the identification peptide is a purification tag configured for isolating and/or enriching a member of the library.

In some embodiments, the phage display library further comprises a solid support. In some embodiments, the solid support houses one or more members of the library. In some embodiments, the phage is an M13 phage, a fl phage, a fd phage, a T4 phage, a T7 phage, a lambda (λ) phage, an MS2 phage, or a ΦX 174 phage.

5.3.6 Production of Phage Display Libraries

Provided herein are methods for producing a phage displaying a lasso peptide component. In certain embodiments, the methods provided herein can produce a large number of phages each displaying a lasso peptide component in a short period of time. In some embodiments, the methods provided herein can produce a plurality of phages displaying diversified species of lasso peptide components simultaneously. Particularly, in some embodiments, the methods provided herein can produce a plurality of phages each displaying a lasso peptide component, wherein the lasso peptide components of the different phages are the same. In some embodiments, the methods provided herein can produce a plurality of phages each displaying a lasso peptide component, wherein each of the lasso peptide components of the plurality of phages is unique. Also provided herein are methods for assembling a plurality of phages displaying diversified species of lasso peptide component into a phage display library.

In various embodiments, the lasso peptide component can assume the form of (i) an intact lasso peptide, (ii) a functional fragment of a lasso peptide, (iii) a lasso precursor peptide, or (iv) a lasso core peptide. A lasso peptide component can undergo transition among the different forms under a suitable condition. For example, when in contact with one or more lasso peptide biosynthesis component (e.g., a lasso peptidase, a lasso cyclase, and/or an RRE), a lasso peptide component in the form of a lasso precursor can be processed into the form of a lasso core peptide, and/or further processed into the form of an intact lasso peptide or a functional fragment of lasso peptide. In some embodiments, neither the non-lasso component of the coat protein nor other components of the phage interferes with either the functional or structural feature of the lasso peptide component.

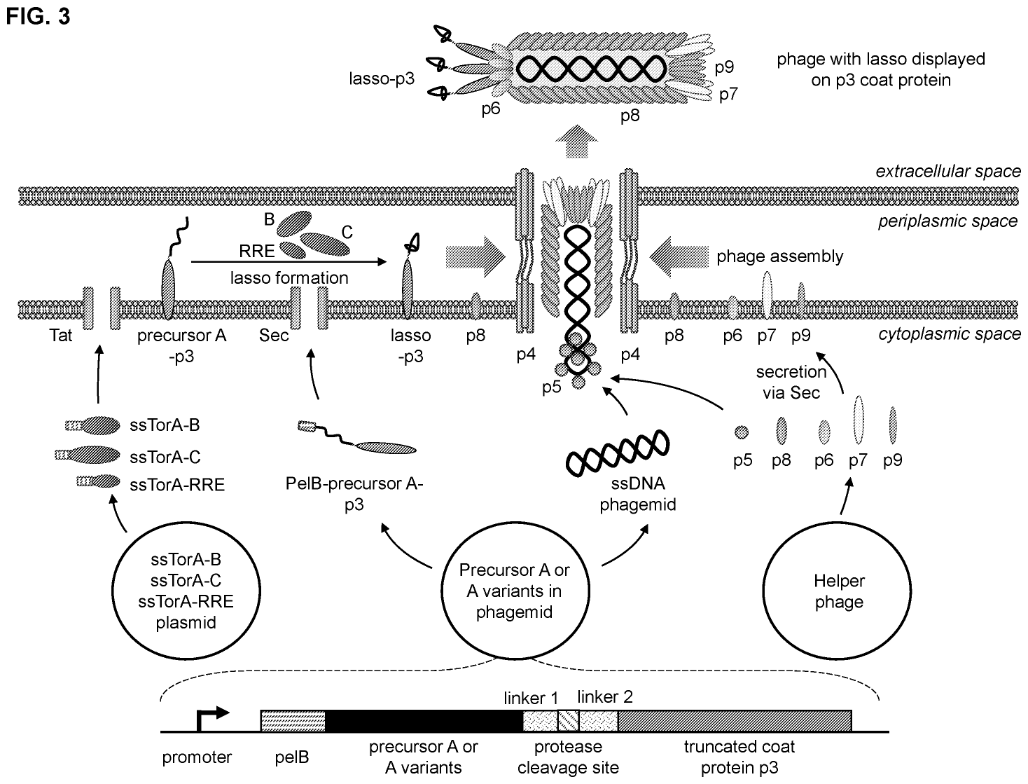

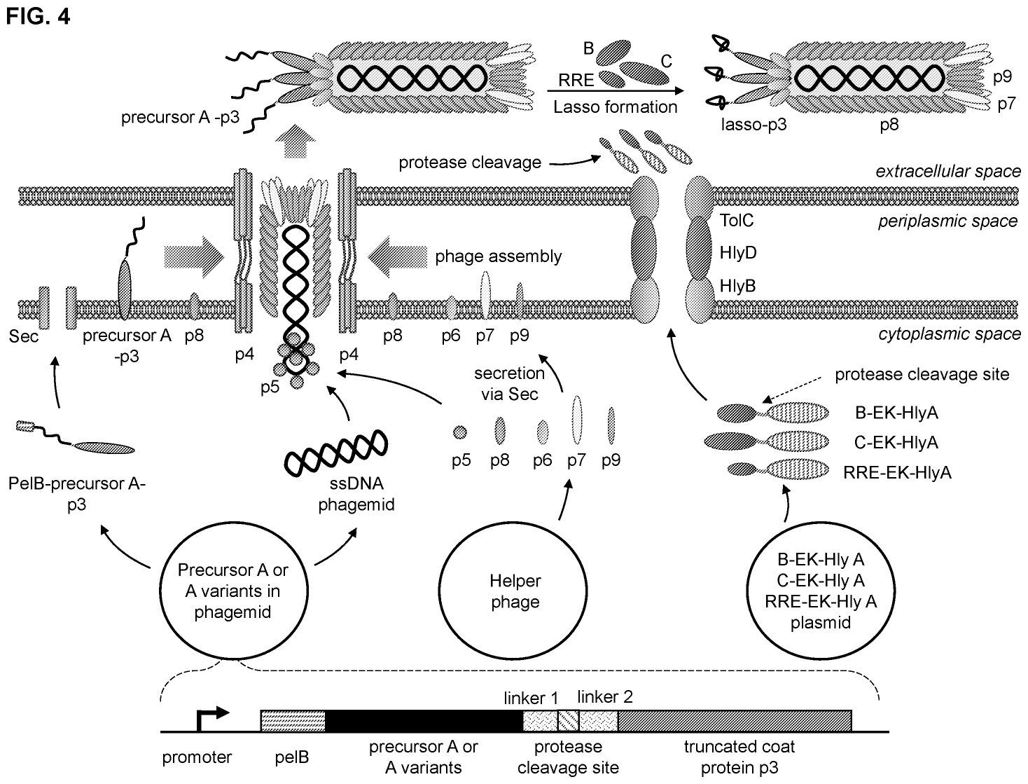

As shown in FIGS. 3 and 4, a lasso-displaying phage can be produced using a suitable host microorganism, such as E. coli. In some embodiments, the method involves providing a system comprising (i) a first nucleic acid sequence encoding one or more structural proteins of a phage; (ii) a phagemid comprising a second nucleic acid sequence encoding a lasso peptide component fused to a phage coat protein; and (iii) a third nucleic acid sequence encoding at least one lasso peptide biosynthesis component. Next, the system is introduced into a population of host cells, such as E. coli cells. Next, the host cells comprising the introduced nucleic acid components can be cultured in a suitable culturing media and under a suitable condition to produce a plurality of phages each displaying a lasso peptide component on a coat protein.

Furthermore, as shown in FIG. 3, in some embodiments, processing the lasso peptide component into lasso peptides having the lariat-like topology can take place in the periplasmic space of the host cell, where the lasso peptide biosynthesis component is transported. Alternatively, as shown in FIG. 4, in some embodiments, processing the lasso peptide component into a lasso peptide having the lariat-like topology can take place extracellularly where the lasso peptide biosynthesis component is secreted. Alternatively, in some embodiments, processing the lasso peptide component into a lasso peptide having the lariat-like structure can take place in the cytoplasm of the host cell, where the lasso peptide biosynthesis component is produced. In any of the embodiments described in this paragraph, the lasso peptide component comprises one or more selected from a lasso peptidase, a lasso cyclase and an RRE.

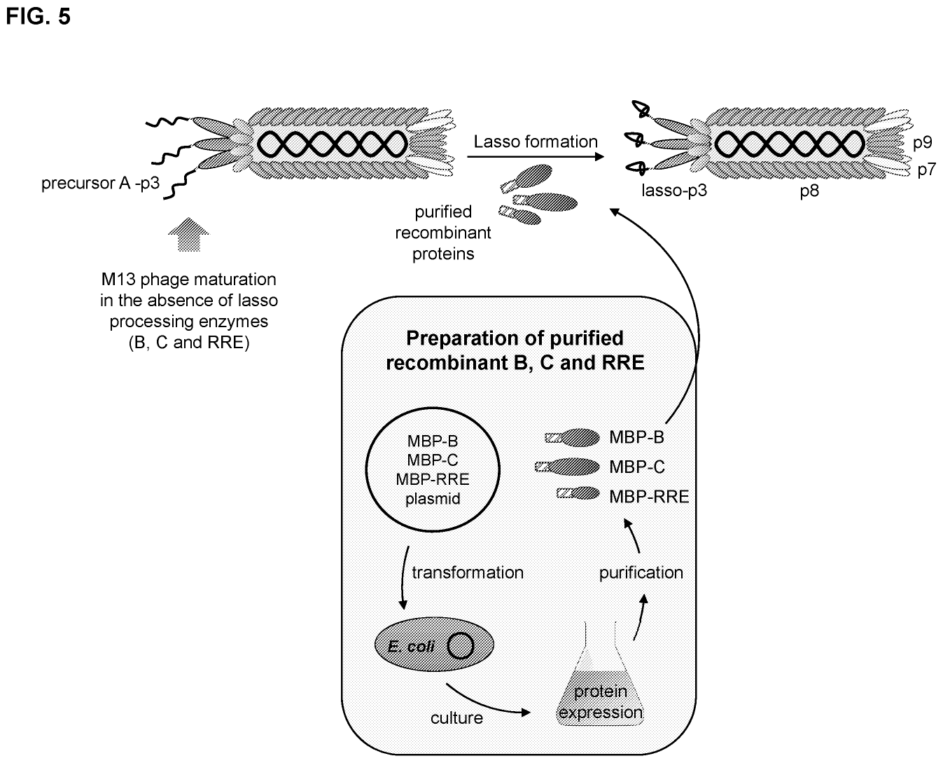

As shown in FIG. 5, a lasso-displaying phage can be produced using a suitable host microorganism, such as E. coli. In some embodiments, the method involves providing a system comprising (i) a first nucleic acid sequence encoding one or more structural proteins of a phage; and (ii) a phagemid comprising a second nucleic acid sequence encoding a lasso peptide component fused to a phage coat protein. Next, the system is introduced into a population of host cells, such as E. coli cells. Next, the host cells comprising the introduced nucleic acid components can be cultured in a suitable culturing media and under a suitable condition to produce a plurality of phages each displaying a lasso peptide component on a coat protein. Next, the produced phages are contacted with lasso peptide biosynthesis components under a suitable condition to process the lasso peptide component into matured lasso peptide having the lariat-like structure. In some embodiments, the phages produced by the host cells are purified from the culturing media before contacted with the lasso peptide biosynthesis components. In some embodiments, lasso peptide biosynthesis components are added into the culture medium to process the lasso peptide component displayed on the phage into matured a lasso peptide having the lariat-like structure. In some embodiments, the lasso peptide biosynthesis component is recombinantly produced by a microorganism. In some embodiments, the lasso peptide biosynthesis component is produced by a cell-free biosynthesis system. In some embodiments, the lasso peptide biosynthesis component is chemically synthesized. In some embodiments, the lasso peptide biosynthesis component is purified before contacted with the phage displaying the lasso peptide component. In any of the embodiments described in this paragraph, the lasso peptide component comprises one or more selected from a lasso peptidase, a lasso cyclase and an RRE.

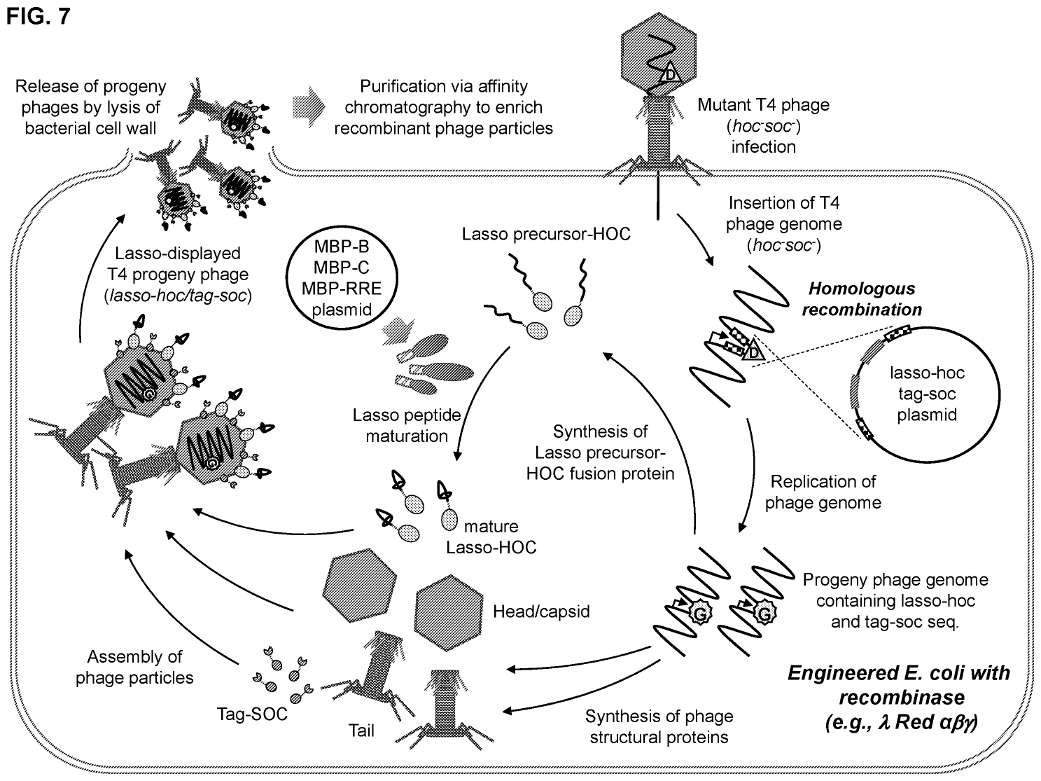

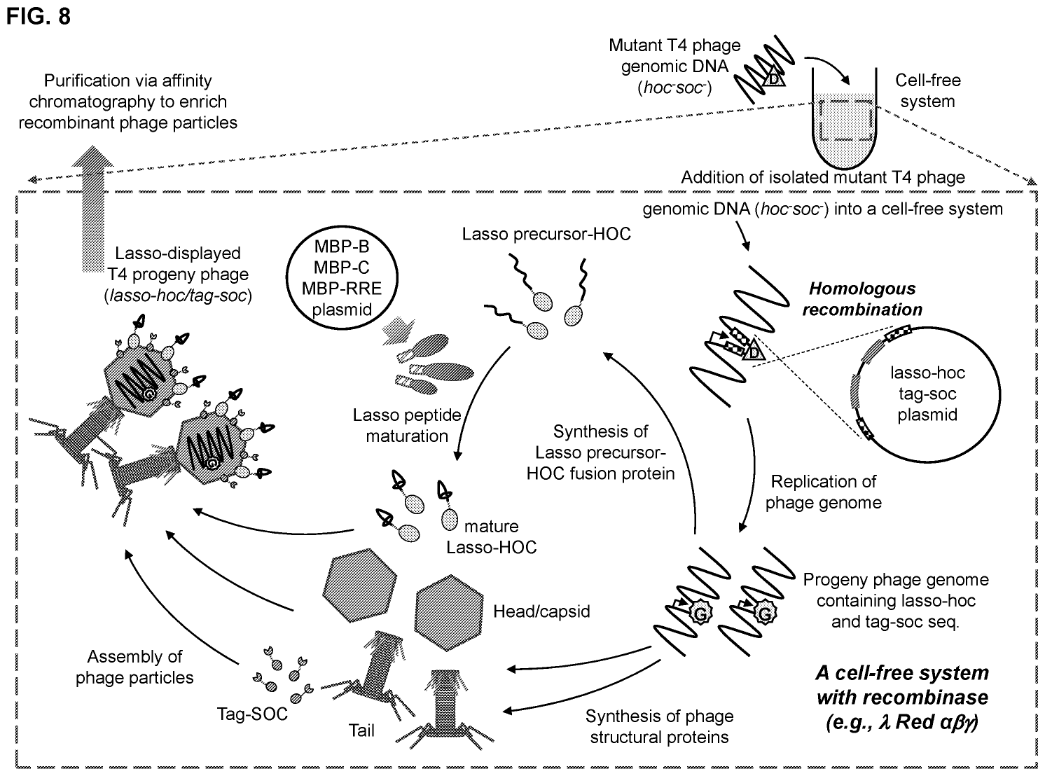

As shown in FIGS. 7 and 8, a lasso-displaying phage can be produced in the cytoplasm of a suitable host microorganism, or in a cell-free biosynthesis reaction mixture. In some embodiments, the method involves providing a system comprising (i) a first nucleic acid sequence encoding one or more structural proteins of a phage; (ii) a second nucleic acid sequence encoding a lasso peptide component fused to a phage coat protein; and (iii) a third nucleic acid sequence encoding at least one lasso peptide biosynthesis component. Next, the system is introduced into a population of host cells, such as E. coli cells. Next, the host cells comprising the introduced nucleic acid components can be cultured in a suitable culturing media and under a suitable condition to produce a plurality of phages each displaying a lasso peptide component on a coat protein.

Particularly, the first and second nucleic acid sequences can be provided in the same nucleic acid molecule. Particularly, in some embodiments, the nucleic acid molecule encodes all essential structural proteins for the phage as well as a fusion protein containing a coat protein. In some embodiments, the nucleic acid molecule encodes both a stand-alone version of the coat protein as well as a fusion protein comprising the coat protein. In some embodiments, the nucleic acid molecule does not encode a stand-alone version of the coat protein, but encodes a fusion protein comprising the coat protein. In some embodiments, the coat protein is nonessential. In some embodiments, the coat protein is nonessential outer capsid protein, such as HOC or SOC of the T4 phage, pX of the T7 phage, pD or pV of a λ (lambda) phage, the MS2 Coat Protein (CP) of an MS2 phage, or the ΦX174 major spike protein G of a ΦX174 phage. In some embodiments, the nucleic acid molecule comprises a mutated phage genome, and can be packaged into the phage capsid formed by the encoded structural proteins.

In some embodiments, sequences encoding the stand-alone version of the coat protein and sequence encoding the fusion protein containing the coat protein are operably linked to the same expression regulatory element. In other embodiments, sequences encoding the stand-alone version of the coat protein and sequence encoding the fusion protein containing the coat protein are operably linked to different expression regulatory elements. Particularly, the expression regulatory elements are selected to control the expression levels, such that the stand-alone version of the coat protein and the fusion protein comprising the coat protein are produced at a desirable ratio by the host cell or in the cell-free biosynthesis reaction mixture.

Alternatively, as shown in FIGS. 7 and 8, in some embodiments, the first and second nucleic acid sequences are provided in separate nucleic acid molecules. Particularly, the separate nucleic acid molecules are configured, upon introducing into the host cell or the cell-free biosynthesis reaction mixture, to produce a recombinant nucleic acid molecule comprising both the first and second nucleic acid sequence. Particularly, in the exemplary embodiments shown in the figures, the first nucleic acid sequence comprises homologous recombination sites flanking the location where the second nucleic acid sequence is to be inserted through recombination. Accordingly, the second nucleic acid sequence is flanked by the homologous recombination sites. Then, a site-specific recombinase or recombinase complex in the cell cytoplasm or cell-free biosynthesis reaction mixture catalyzes homologous recombination between the two molecules to produce the recombinant nucleic acid molecule comprising both the first and second nucleic acid sequences. In some embodiments, the functionality of the recombinase is provided by the host cell or the cell-free biosynthesis reaction mixture. In other embodiments, the present system further comprises components for providing the functionality of the recombinase.

In some embodiments, the first nucleic acid sequence is configured to be packaged into the phage capsid formed by the encoded structural proteins. In some embodiments, the first nucleic acid sequence comprises the phage genome and can be assembled into the capsid formed by the encoded structural proteins. In some embodiments, the phage genome is wild-type. In other embodiments, the phage genome is mutated.

In particular embodiments, the mutated phage genome sequence does not encode a stand-alone version of a phage coat protein that is selected for displaying other peptide or protein components. Particularly, in some embodiments, the mutated phage genome has one or more null mutations in the endogenous sequence encoding the coat protein. For example, in some embodiments, the endogenous sequence encoding the coat protein is deleted from the phage genome. In some embodiments, a sequence encoding the stand-alone version of the coat protein is replaced by the second nucleic acid sequence encoding the fusion protein comprising the coat protein during the recombination process. In some embodiments, the recombinant nucleic acid molecule is capable of being packaged into the phage capsid formed by the encoded structural proteins.

In particular embodiments, the mutated phage genome encodes both a stand-alone version of the coat protein as well as a fusion protein comprising the coat protein. In other embodiments, sequences encoding the stand-alone version of the coat protein and sequence encoding the fusion protein containing the coat protein are operably linked to different expression regulatory elements. Particularly, the expression regulatory element are selected to control the expression levels, such that the stand-alone version of the coat protein and the fusion protein comprising the coat protein are produced at a desirable ratio by the host cell or in the cell-free biosynthesis reaction mixture.

In some embodiments, the genotype of the phage produced as described herein at matches at least partially the phenotype of the phage. In these embodiments, the lasso peptide component displayed on the phage can be identified by analyzing genetic materials of the phage. Accordingly, in some of these embodiments, identification of the lasso peptide component displayed on a phage depends on packaging into the phage capsid a nucleic acid sequence encoding the lasso peptide component. As described herein, in some embodiments, the second nucleic acid sequence encoding the fusion protein comprising the lasso peptide component is packaged into the phage capsid. In some embodiments, a nucleic acid molecule comprising both the first and second nucleic acid sequences are packaged into the phage capsid.

In other embodiments, the genotype of the phage produced as described herein does not match the phenotype of the phage. In some of these embodiments, an identification mechanism is provided for identifying and/or manipulating the phage, and the lasso peptide component displayed on the phage. For example, in some embodiments, the second nucleic acid sequence further encodes a fusion protein comprising an identification peptide fused to a coat protein of the phage. In various embodiments, the identification peptide is configured to identify and/or manipulate the phage displaying the identification peptide, as well as the lasso peptide component also displayed on the phage. For example, the identification peptide can produce a unique detectable signal identifying the phage or the lasso peptide component. The identification peptide can be a purification tag for isolating and/or enriching the population of phages displaying a lasso peptide component. In another exemplary embodiment, the process for making the phage takes place at a unique location, and the location information can be used to identify the phage and the lasso peptide component displayed thereon. For example, in some embodiments, the lasso-displaying phage is produced in a well of a multi-well plate that is assigned with a unique well ID number.

Accordingly, in some of these embodiments, identification of the lasso peptide component displayed on a phage does not require packaging into the phage capsid a nucleic acid sequence encoding the lasso peptide component. Thus, in some embodiments, the second sequence encoding the fusion protein comprising the lasso peptide component is not packaged into the phage capsid. For example, in some embodiments, the second sequence does not contain a packaging signal. In some embodiments, the second sequence is not part of a sequence containing a packaging signal.

In particular embodiments, the first nucleic acid sequence is provided in the form of an expression vector. In some embodiments, the second nucleic acid sequence is provided in the form of an expression vector. In some embodiments, both the first and second nucleic acid sequences are provided in the same expression vector. In some embodiments, the vector containing the first and/or second nucleic sequence is a plasmid. In some embodiments, the phage structural proteins assembled into an empty capsid without any genome sequence, and the phage displays a lasso peptide component on the capsid.

In particular embodiments, the first nucleic acid sequence but not the second nucleic acid sequence is packaged into the phage capsid, and the phage displays a lasso peptide component on the capsid. In some embodiments, the first nucleic acid sequence comprises a wild-type genome of the phage. In some embodiments, the first nucleic acid sequence comprises a mutated genome of the phage having a null mutation in an endogenous sequence encoding the coat protein. In particular embodiments, the endogenous sequence encoding the coat protein is deleted from the genome.

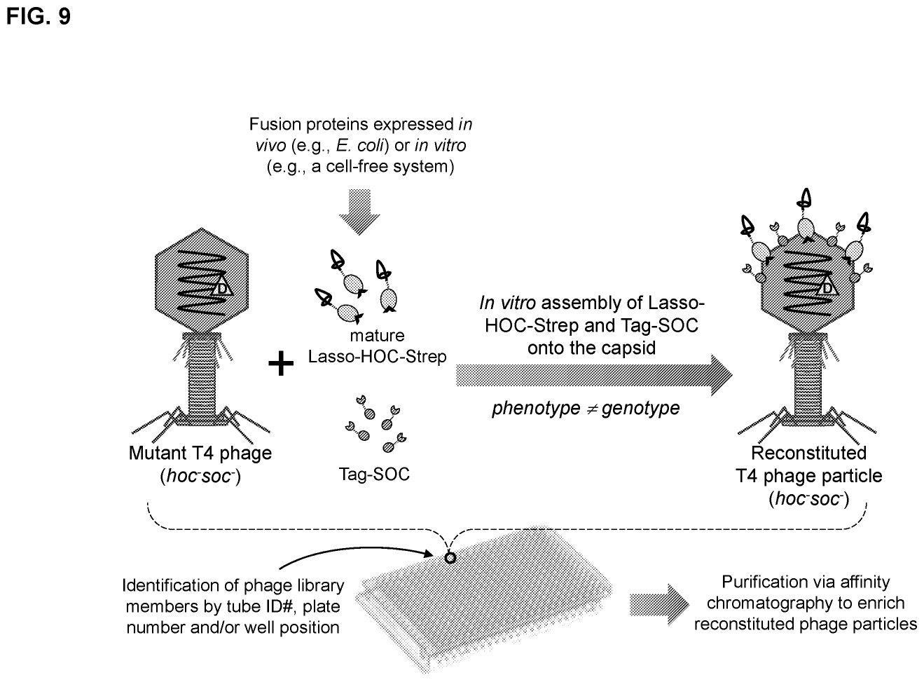

As shown in FIG. 9, a lasso-displaying phage can be produced in vitro by contacting a partially assembled phage capsid with a fusion protein comprising the lasso peptide component fused to a selected coat protein of the phage. Particularly, in some embodiments, the selected coat protein is a nonessential outer capsid protein.

Without being bound by the theory, it is contemplated that in certain phage species only a maximum number of copies of a coat protein can be assembled into one capsid. For example, T4 phage capsid is decorated with 155 copies of Hoc. (Sathaliyawala et al. Journal of Virology, August, 2006, pp. 7688-7698). Thus, in some embodiments, the partially assembled phage capsid is devoid of the selected coat protein, and contacting the partially assembled phage capsid with a population of fusion proteins comprising the coat protein leads to the assembly of up to the maximum number of the fusion proteins onto the phage capsid.

It is also contemplated that the density of the fusion proteins on the phage capsid can be controlled in various ways. For example, to reduce the density of the fusion proteins on the phage capsid, in some embodiments, the partially assembled phage capsid contains some but less than the maximum number of the coat proteins, and contacting the partially assembled phage capsid with a population of fusion proteins comprising the coat protein leads to the assembly of less than the maximum number of copies of the fusion proteins onto the phage capsid.

In some embodiment, to reduce the density of the fusion proteins on the phage capsid, the partially assembled phage capsid devoid of the coat protein is contacted with a mixture containing both the stand-alone version of the coat proteins and the fusion protein containing the coat protein. In these embodiments, the stand-alone coat proteins compete with the fusion proteins for assembling onto the phage capsid, and lead to assembly of less than the maximum number of copies of the fusion protein on the phage capsid.

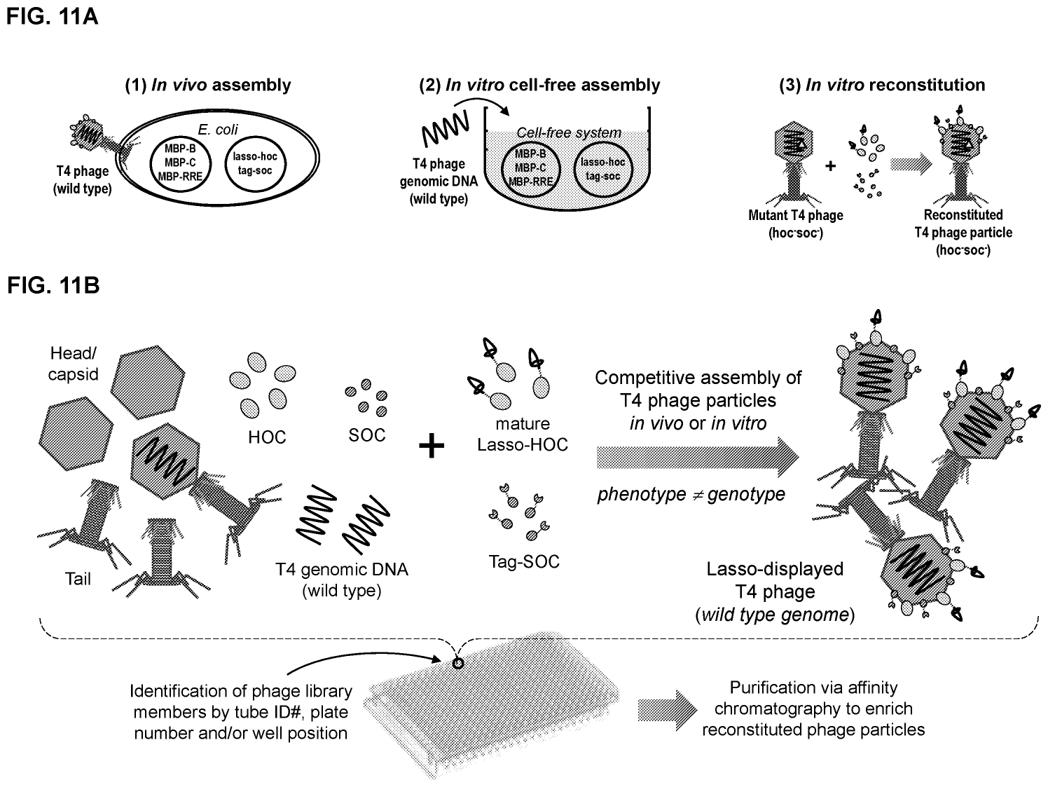

In particular embodiments, such as shown in the first and second panels of FIGS. 11A, competitive assembly of both a stand-alone coat protein and a fusion protein containing the coat protein can be performed in vivo in a host cell or in vitro using a cell-free biosynthesis reaction mixture. Particularly, as shown in FIG. 11B, a wild-type genome of a phage is introduced into a host cell or a cell-free biosynthesis reaction mixture to produce encoded phage proteins, including a first coat protein of the phage. Also introduced into the host cell or cell-free biosynthesis reaction mixture is a second nucleic acid sequence encoding a fusion protein comprising a lasso peptide component fused to the first coat protein. The encoded phage proteins produced in the cell cytoplasm or cell-free biosynthesis reaction mixture assemble into the capsid in the presence of the fusion protein expressed from the second nucleic acid sequence. Thus, the stand-alone coat protein and the fusion protein compete for assembly on the phage capsid. In some embodiments, the phage is a T4 phage, and the coat protein is HOC or SOC.

In other embodiments, such as shown in the third panel of FIG. 11A, competitive assembly of both a stand-alone coat protein and a fusion protein containing the coat protein can be performed in vitro by mixing isolated partially assembled phage capsids and protein components together. Particularly, as shown in the figure, the partially assembled phage capsid does not contain a nucleic acid sequence encoding the lasso peptide component in the fusion protein. Particularly, in some embodiments, the partially assembled phage capsid contains a mutated genome devoid of endogenous sequence encoding the coat protein. In some embodiments, the partially assembled phage capsid is produced by introducing a mutated phage genome sequence that does not encode the coat protein into a host cell or a cell-free biosynthesis reaction mixture, followed by culturing the host cell or incubating the cell-free biosynthesis reaction mixture under a suitable condition to produce the partially assembled phage capsid. The partially assembled phage capsid is then isolated and contacted with a mixture of both stand-alone coat proteins and fusion proteins comprising the coat protein for competitive assembly.

Other methods for controlling the fusion protein density can be envisioned by those of ordinary skills in the art based on the present disclosure. For example, controlling the density of the fusion protein on the phage capsid can be achieved by adjusting the concentration of the partially assembled phage particles and/or the concentration of the fusion proteins that are contacted together. For example, controlling the density of the fusion protein on the phage capsid can be achieved by adjusting the incubation time during which the partially assembled phage capsid and the fusion protein is contacted. For example, controlling the density of the fusion protein on the phage capsid can be achieved by adjusting the ratio of the stand-alone coat protein and the fusion protein in the mixture contacted with the partially assembled phage capsid.

In various embodiments, the partially assembled phage capsid is further contacted with a fusion protein comprising an identification peptide fused to a coat protein of the phage. In some embodiments, the identification peptide is a purification tag. In some embodiments, the identification peptide produces a detectable signal. In some embodiments, the identification peptide and the lasso peptide components are fused to the same coat protein of the phage. In other embodiments, the identification peptide and the lasso peptide components are fused to different coat proteins of the phage. In various embodiments, contacting the partially assembled phage capsid with one or more fusion proteins occurs in a unique location on a solid support, such as in a well of a multi-well plate.

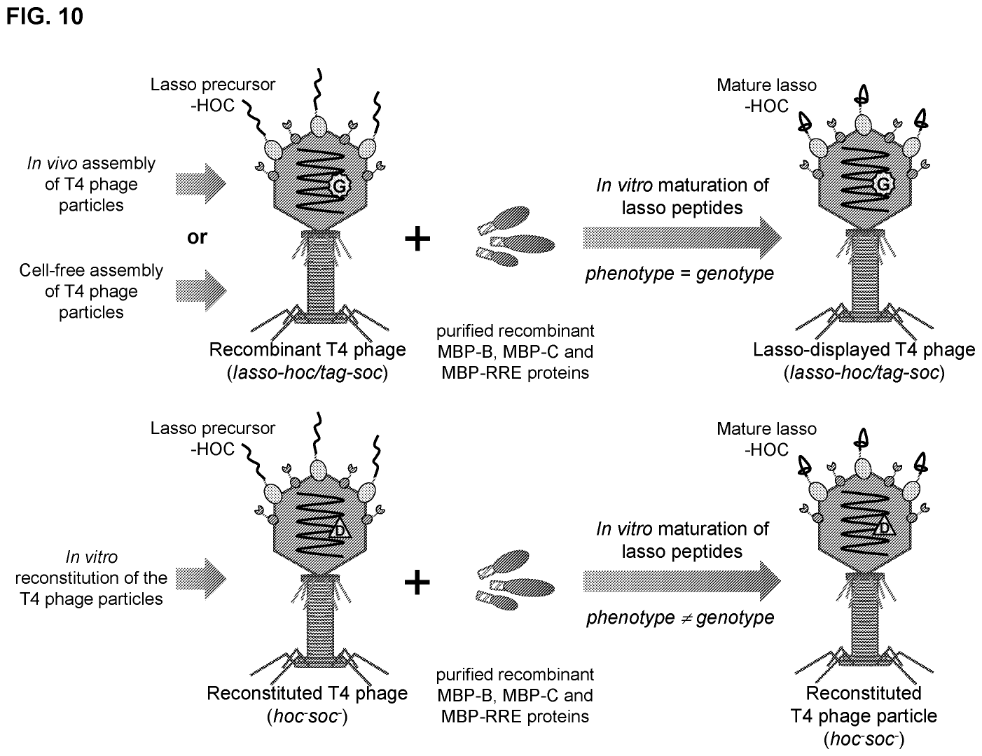

As shown in FIG. 10, the lasso peptide component displayed on the phage capsid can be processed by at least one lasso peptide biosynthesis component into a lasso peptide or a functional fragment of lasso peptide. Particularly, in some embodiments, the lasso maturation step can occur in a host cell cytoplasm or a cell-flee biosynthesis reaction mixture where the phage components are expressed and assembled. A third nucleic acid molecule encoding at least one lasso peptide biosynthesis components can be introduced into the same host cell or the cell-free biosynthesis reaction mixture. The lasso peptide biosynthesis components produced in the cell cytoplasm of cell-free biosynthesis reaction mixture then process a lasso precursor peptide or lasso core peptide displayed on the phage capsid into a lasso peptide or functional fragment of lasso peptide. Alternatively, in some embodiments, such as shown in FIG. 5 or FIG. 10 (bottom), a lasso-displaying phage are isolated before contacting with the lasso peptide biosynthesis components. In some embodiments, lasso peptide biosynthesis components are added into the culture medium to process the lasso peptide component displayed on the phage into matured a lasso peptide having the lariat-like structure. In some embodiments, the lasso peptide biosynthesis component is recombinantly produced by a microorganism. In some embodiments, the lasso peptide biosynthesis component is produced by a cell-free biosynthesis system. In some embodiments, the lasso peptide biosynthesis component is chemically synthesized. In some embodiments, the lasso peptide biosynthesis component is purified before contacted with the phage displaying the lasso peptide component. In any of the embodiments described in this paragraph, the lasso peptide component comprises one or more selected from a lasso peptidase, a lasso cyclase and an RRE.

In various embodiments described herein, one or more of the nucleic acid sequence to be introduced into the host cell encodes a fusion protein. For example, in some embodiments, the nucleic acid sequence encodes a fusion protein comprising a lasso peptide component fused to a phage coat protein. In particular embodiments, the lasso peptide component is fused to the phage coat protein via a linker. In some embodiments, the fusion protein comprises the lasso peptide component fused to a secretion signal. In particular embodiments, the lasso peptide component is fused to a secretion signal via a linker. In some embodiments, the fusion protein comprises the phage coat protein fused to the secretion signal. In particular embodiments, the phage coat protein is fused to the secretion signal via a linker.

For example, in some embodiments, the nucleic acid sequence encodes a fusion protein comprising a lasso peptide biosynthesis component fused to a secretion signal. In particular embodiments, the lasso peptide biosynthesis component is fused to a secretion signal via a linker. Particularly, in some embodiments, the fusion protein comprises a lasso peptidase fused to a secretion signal. In particular embodiments, the lasso peptidase is fused to a secretion signal via a linker. In some embodiments, the fusion protein comprises a lasso cyclase fused to a secretion signal. In particular embodiments, the lasso cyclase is fused to a secretion signal via a linker. In some embodiments, the fusion protein comprises an RRE fused to a secretion signal. In particular embodiments, the RRE is fused to the secretion signal via a linker.

For example, in some embodiments, the nucleic acid sequence encodes a fusion protein comprising a lasso peptide biosynthesis component fused to a purification tag. In particular embodiments, the lasso peptide biosynthesis component is fused to a purification tag via a linker. Particularly, in some embodiments, the fusion protein comprises a lasso peptidase fused to a purification tag. In particular embodiments, the lasso peptidase is fused to a purification tag via a linker. In some embodiments, the fusion protein comprises a lasso cyclase fused to a purification tag. In particular embodiments, the lasso cyclase is fused to a purification tag via a linker. In some embodiments, the fusion protein comprises an RRE fused to a purification tag. In particular embodiments, the RRE is fused to the purification tag via a linker.

For example, in some embodiments, the nucleic acid sequence encodes a fusion protein comprising two or more lasso peptide biosynthesis components fused to each other. In particular embodiments, the two or more lasso peptide biosynthesis components are fused to each other via a linker. Particularly, in some embodiments, the fusion protein comprises a lasso cyclase fused to a lasso peptidase. In particular embodiments, the lasso cyclase is fused to the lasso peptidase via a linker. In some embodiments, the fusion protein comprises a lasso peptidase fused to an RRE via a linker. In particular embodiments, the lasso peptidase is fused to an RRE via a linker. In some embodiments, the fusion protein comprises a lasso cyclase fused to an RRE. In particular embodiments, the lasso cyclase is fused to an RRE via a linker.

In any of the embodiments described in the above paragraph, the fusion protein may further comprise a purification tag or a secretion signal fused to the lasso peptide biosynthesis component via a linker. For example, in some embodiments, the fusion protein comprises a lasso cyclase, a lasso peptidase and a purification tag. Particularly, in some embodiments, the lasso cyclase is fused to a lasso peptidase via a linker, and further the lasso cyclase or the lasso peptidase is fused to the purification tag via a linker. For example, in some embodiments, the fusion protein comprises a lasso cyclase, an RRE and a secretion signal. Particularly, in some embodiments, the lasso cyclase is fused to the RRE via a linker, and further the lasso cyclase or the RRE is fused to the secretion signal via a linker. For example, in some embodiments, the fusion protein comprises a lasso peptidase, an RRE and a purification tag. Particularly, in some embodiments, the lasso peptidase is fused to the RRE via a linker, and further the lasso peptidase or the RRE is fused to the purification tag via a linker. For example, in some embodiments, the fusion protein comprises a lasso peptidase, an RRE and a secretion signal. Particularly, in some embodiments, the lasso peptidase is fused to the RRE via a linker, and further the lasso peptidase or the RRE is fused to the secretion signal via a linker. For example, in some embodiments, the fusion protein comprises a lasso peptidase, a lasso cyclase, an RRE and a purification tag. Particularly, in some embodiments, one or more connections between the lasso peptidase, lasso cyclase, RRE and/or purification tag is via a linker. For example, in some embodiments, the fusion protein comprises a lasso peptidase, a lasso cyclase, an RRE and a secretion signal. Particularly, in some embodiments, one or more connections between the lasso peptidase, lasso cyclase, RRE and/or secretion signal is via a linker.

The linker used in any of the embodiments described herein can be a cleavable peptidic linker. Exemplary endo- and exo-proteases that can be used for cleaving the peptidic linker and thus the separation of the different domains of the fusion proteins include but are not limited to Enteropeptidase, Enterokinase, Thrombin, Factor Xa, TEV protease, Rhinovirus 3C protease; a SUMO-specific and aNEDD8-specific protease from Brachypodium distachyon (bdSENP1 and bdNEDP1), the NEDP1 protease from Salmo salar (ssNEDP1), Saccharomyces cerevisiae Atg4p (scAtg4) and Xenopus laevis Usp2 (xlUsp2). Additional examples of proteases and their recognition site (i.e., sequences that can be used to form the peptidic linker) for cleavage can be found in Waugh Protein Expr Purif. 2011 December; 80(2): 283-293. In some embodiments, commercially available proteases and corresponding recognition site sequences can be used in connection with the present disclosure.

The purification tag used in any of the embodiments described herein can be selected from Albumin-binding protein (ABP), Alkaline Phosphatase (AP), AU1 epitope, AU5 epitope, Bacteriophage 17 epitope (17-tag), Bacteriophage V5 epitope (V5 tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S-transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg tag), Polyaspartate (Asp-tag), Polycysteine (Cys-tag), Polyhistidine (His-tag), Polyphenylalanine (Poly-tag), Profinity eXact™, Protein C, S1 tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, or VSV-G.

### 5.3.6.1 Genomic Mining Tools for Genes Coding Natural Lasso Peptides

According to the present disclosure, nucleic acid sequences encoding the lasso peptide component and/or the lasso peptide biosynthesis component can derive from naturally existing lasso peptide biosynthetic gene clusters.

Some naturally existing lasso peptides are encoded by a lasso peptide biosynthetic gene cluster, which typically comprises three main genes: one encodes for a lasso precursor peptide (referred to as Gene A), and two encode for processing enzymes including a lasso peptidase (referred to as Gene B) and a lasso cyclase (referred to as Gene C). The lasso precursor peptide comprises a lasso core peptide and additional peptidic fragments known as the “leader sequence” that facilitates recognition and processing by the processing enzymes. The leader sequence may determine substrate specificity of the processing enzymes. The processing enzymes encoded by the lasso peptide gene cluster convert the lasso precursor peptide into a matured lasso peptide having the lariat-like topology. Particularly, the lasso peptidase removes additional sequences from the precursor peptide to generate a lasso core peptide, and the lasso cyclase cyclizes a terminal portion of the core peptide around a terminal tail portion to form the lariat-like topology. Some lasso gene clusters further encodes for additional protein elements that facilitates the post-translational modification, including a facilitator protein known as the post-translationally modified peptide (RiPP) recognition element (RRE). Some lasso gene clusters further encodes for lasso peptide transporters, kinases, acetyltransferases, or proteins that play a role in immunity, such as isopeptidase. (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C. D., et al., Angew. Chem. Int. Ed., 2016, 55, 12717-12721; Zhu, S., et al., J. Biol. Chem. 2016, 291, 13662-13678; Zong, C. et al., Chem Commun (Camb), 2018; 54(11), 1339-1342).

Computer-based genome-mining tools can be used to identify lasso biosynthetic gene clusters based on known genomic information. For example, one algorithm known as RODEO can rapidly analyze a large number of biosynthetic gene clusters (BGCs) by predicting the function for genes flanking query proteins. This is accomplished by retrieving sequences from GenBank followed by analysis with HMMER3. The results are compared against the Pfam database with the data being returned to the users in the form of spreadsheet. For analysis of BGCs not encoding proteins not covered by Pfam, RODEO allows usage of additional pH Ms (either curated databases or user-generated). Taking advantage of RODEO's ability to rapidly analyze genes neighboring a query, it is possible to compile a list of all observable lasso peptide biosynthetic gene clusters in GeneBank (Online Methods). A comprehensive evaluation of this data set would provide great insight into the lasso peptide family. Lasso peptide biosynthetic gene clusters can be identified by looking for the local presence of genes encoding proteins matching the Pfams for the lasso cyclase, lasso peptidase, and RRE.

To confidently predict lasso precursors, RODEO next performed a six-frame translation of the intergenic regions within each of the identified potential lasso biosynthetic gene clusters. The resulting peptides can be assessed based on length and essential sequence features and split into predicted leader and core regions. A series of heuristics based on known lasso peptide characteristics can be defined to predict precursors from a pool of false positives. After optimization of heuristic scoring, good prediction accuracy for biosynthetic gene clusters closely related to known lasso peptides can be obtained.

Machine learning, particularly, support vector machine (SVM) classification, would be effective in locating precursor peptides from predicted BGCs more distant to known lasso peptides. SVM is well-suited for RiPP discovery due to availability of SVM libraries that perform well with large data sets with numerous variables and the ability of SVM to minimize unimportant features. The SVM classifier can be optimized using a randomly selected and manually curated training set from the unrefined whole data. Of these, a random subpopulation was withheld as a test set to avoid over-fitting. By combining SVM classification with motif (MEME) analysis, along with our original heuristic scoring, prediction accuracy was greatly enhanced as evaluated by recall and precision metrics. This tripartite procedure can yield a high-scoring, well-separated population of lasso precursor peptide from candidate peptides. The training set was found to display nearly identical scoring distributions upon comparison to the full data set.

Other examples of genomic or biosynthetic gene search engine that can be used in connection with the present disclosure include the WARP DRIVE BIO™ software, anti-SMASH (ANTI-SMASH™) software (See: Min, K., et al., Nucleic Acids Res., 2017, 45, W36-W41), iSNAP™ algorithm (See: Ibrahim, A., et al., Proc. Nat. Acad. Sci., USA., 2012, 109, 19196-19201), CLUSTSCAN™ (Starcevic, et al., Nucleic Acids Res., 2008, 36, 6882-6892), NP searcher (Li et al. (2009) Automated genome mining for natural products. BMC Bioinformatics, 10, 185), SBSPKS™ (Anand, et al. Nucleic Acids Res., 2010, 38, W487 W496), BAGEL3™ (Van Heel, et al., Nucleic Acids Res., 2013, 41, W448-W453), SMURF™ (Khaldi et al., Fungal Genet. Biol., 2010, 47, 736-741), ClusterFinder (CLUSTERFINDER™) or ClusterBlast (CLUSTERBLAST™) algorithms, and an Integrated Microbial Genomes (IMG)-ABC system (DOE Joint Genome Institute (JGI)). In some embodiments, lasso peptide biosynthetic gene clusters for use in CFB methods and processes as provided herein are identified by mining genome sequences of known bacterial natural product producers using established genome mining tools, such as anti-SMASH, BAGEL3, and RODEO. These genome mining tools can also be used to identify novel biosynthetic genes within metagenomic based DNA sequences. Lasso peptide biosynthetic gene clusters can be used in the methods and systems described herein to produce various lasso peptides and libraries of lasso peptides.

5.3.6.2 Diversifying Lasso Peptides

In some embodiments, the present system and methods are configured to produce a phage display library comprising a plurality of distinct species of lasso peptide component. In some embodiments, the present systems are used to facilitate the creation of mutational variants of lasso peptides using methods involving, for example, the synthesis of codon mutants of the lasso precursor peptide or lasso core peptide gene sequence. Lasso precursor peptide or lasso core peptide gene or oligonucleotide mutants can be introduced into the host organism, thus enabling the creation of a phage population displaying highly diversified lasso peptide components. In some embodiments, the present system and methods are used to facilitate the creation of large mutational lasso peptide libraries using, for example, site-saturation mutagenesis and recombination methods. In some embodiments, the present system and method are used to facilitate the creation of mutational variants of lasso peptides by introducing non-natural amino acids into the core peptide sequence, followed by formation of the lasso structure as described herein.

Without being bound by the theory, it is contemplated that different lasso peptidase can process the same lasso precursor peptide into different lasso core peptide by recognizing and cleaving different leader peptide off the lasso precursor. Additionally, different lasso cyclase can process the same lasso core peptide into distinct lasso peptides by cyclizing the core peptide at different ring-forming amino acid residues. Additionally, different RREs can facilitate different processing by the lasso peptidase and/or lasso cyclase, and thus lead to formation of distinct lasso peptides from the same lasso precursor peptide.

Accordingly, in some embodiments, to produce a natural lasso peptide, the nucleic acid sequences encoding the lasso precursor peptide, lasso peptidase, and lasso cyclase are derived from the same lasso peptide biosynthetic gene cluster (such as Genes A, B, and C of the same lasso peptide biosynthetic gene cluster). In some embodiments, to produce a natural lasso peptide, the nucleic acid sequences encoding the lasso precursor peptide, lasso peptidase, lasso cyclase, and RRE are derived from coding sequences of the same lasso peptide biosynthetic gene cluster.

In some embodiments, to produce a natural lasso peptide, the nucleic acid sequences coding the lasso core peptide, and lasso cyclase are derived from coding sequences of the same lasso peptide biosynthetic gene cluster (such as Genes A and C of the same lasso peptide biosynthetic gene cluster). In some embodiments, to produce a natural lasso peptide, the nucleic acid sequences coding the lasso core peptide, lasso cyclase, and RRE are derived from coding sequences of the same lasso peptide biosynthetic gene cluster.

In alternative embodiments, to produce a derivative of a natural lasso peptide, at least two of the nucleic acid sequences encoding the lasso precursor peptide, lasso peptidase and lasso cyclase are derived from coding sequences of different lasso peptide biosynthetic gene clusters (such as Gene A from one, and Genes B and C from another, lasso peptide biosynthetic gene cluster). In alternative embodiments, to produce a derivative of a natural lasso peptide, at least two of the nucleic acid sequences encoding the lasso precursor peptide, lasso peptidase, lasso cyclase and RRE are derived from coding sequences of different lasso peptide biosynthetic gene clusters.

In alternative embodiments, to produce a derivative of a natural lasso peptide, the nucleic acid sequences encoding the lasso core peptide and lasso cyclase are derived from coding sequences of different lasso peptide biosynthetic gene clusters (such as Gene A from one, and Gene C from another, lasso peptide biosynthetic gene cluster). In alternative embodiments, to produce a derivative of a natural lasso peptide, at least two of the nucleic acid sequences encoding the lasso core peptide, lasso cyclase and RRE are derived from coding sequences of different lasso peptide biosynthetic gene clusters.

In some embodiments, the coding sequences derived from the lasso peptide biosynthesis component are mutated in order to further diversify the lasso peptide species presented in the phage display library.

In some embodiments, the nucleic acid sequence coding for the lasso peptide component is derived from a natural sequence, such as a Gene A sequence or open reading frame thereof. In some embodiments, a plurality of nucleic acid sequences coding for the lasso peptide component are derived from the same or different natural sequences. In specific embodiments, derivation of a nucleic acid sequence (e.g., a Gene A sequence) is performed by introducing one or more mutation(s) to the nucleic acid sequence. In various embodiments, the one or more mutation(s) are one or more selected from amino acid substitution, deletion, and addition. In various embodiments, the one or more mutation(s) can be introduced using mutation methods described herein and/or known in the art.

Particularly, in specific embodiments, a plurality of coding sequences each encoding a different lasso peptide component is provided. In some embodiments, the plurality of coding sequences comprise sequences from a plurality of different lasso peptide biosynthetic gene clusters (such as a plurality of different Gene A sequences or open reading flames thereof). In some embodiments, the plurality of coding sequences are derived from one or more Gene A sequences or open reading frames thereof.

In some embodiments, the plurality of coding sequences are derived from the same Gene A sequence or open reading flame thereof. In specific embodiments, to produce a library comprising diversified species of lasso peptides, a coding sequence of lasso precursor peptide of interest is mutated to produce a plurality of coding sequences encoding lasso peptide components having different amino acid sequences. In some embodiments, a lasso peptide having one or more desirable target properties is selected, and its corresponding precursor peptide is used as the initial scaffold to generate the diversified species of precursor peptides in a library. In some embodiments, one or more mutation(s) are introduced by methods of directed mutagenesis. In alternative embodiments, one or more mutation(s) are introduced by methods of random mutagenesis.

Without being bound by the theory, it is contemplated that the leader sequence of a lasso precursor peptide is recognized by the lasso processing enzymes and can determine specificity and selectivity of the enzymatic activity of the lasso peptidase or lasso cyclase. Accordingly, in some embodiments, only the core peptide portion of the lasso precursor peptide is mutated, while the leader sequence remains unchanged. In some embodiments, the leader sequence of a lasso precursor peptide is replaced by the leader sequence of a different lasso precursor peptide.

Without being bound by theory, it is contemplated that certain lasso cyclases can cyclize the lasso core peptide by joining the N-terminal amino group with the carboxyl group on side chains of glutamate or aspartate residue located at the 7^{th}, 8^{th} or 9^{th} position (counting from the N-terminus) in the core peptide. Accordingly, in some embodiments, random mutations can be introduced to any amino acid residues in a lasso core peptide, or a core peptide region of a lasso precursor peptide, except that at least one of the 7^{th}, 8^{th} or 9^{th} positions (counting from the N-terminus) in the lasso core peptide or core peptide region of a lasso precursor has a glutamate or aspartate residue. In some embodiments, a glutamate residue is introduced to the 7^{th}, 8^{th} or 9^{th} positions (counting from the N-terminus) in the lasso core peptide or core peptide region of a lasso precursor by amino acid addition or amino acid substitution mutations using the methods described herein and/or known in the art. In some embodiments, an aspartate residue is introduced to the 7^{th}, 8^{th} or 9^{th} positions (counting from the N-terminus) in the lasso core peptide or core peptide region of a lasso precursor by amino acid addition or amino acid substitution mutations using the methods described herein and/or known in the art.

Without being bound by theory, it is contemplated that intra-peptide disulfide bond(s), including one or more disulfide bonds (i) between the loop and the ring portions, (ii) between the ring and tail portions, (iii) between the loop and tail portions, and/or (iv) between different amino acid residues of the tail portion of a lasso peptide can contribute to maintain or improve stability of the lariat-like topology of a lasso peptide. Accordingly, in some embodiments, a lasso core peptide or lasso precursor peptide is engineered to have at least two cysteine residues. In specific embodiments, at least two cysteine residues locate on the loop and ring portions of a lasso peptide, respectively. In specific embodiments, at least two cysteine residues locate on the ring and tail portions of a lasso peptide, respectively. In specific embodiments, the at least two cysteine residues locate on the loop and tail portions of a lasso peptide, respectively. In specific embodiments, at least two cysteine residues locate on tail portion of a lasso peptide, respectively. In various embodiments, one or more cysteine residues as described herein are introduced to the nucleic acid sequence of a lasso peptide by amino acid addition or amino acid substitution mutations using the methods described herein and/or known in the art.

Without being bound by theory, it is contemplated that steric effects (e.g., steric hindrance) can contribute to maintain or improve stability of the lariat-like topology of a lasso peptide. Accordingly, in some embodiments, amino acid residues having sterically bulky side chains are located and/or introduced to the locations in the lasso core peptide or the core peptide region of a lasso precursor peptide that are in close proximity to the plane of the ring. In some embodiments, at least one amino acid residue(s) having sterically bulky side chains are located and/or introduced to the tail portion of the lasso peptide. In particular embodiments, multiple bulky amino acids can be consecutive amino acid residues in the tail portion of the lasso peptide. The bulky amino acid residue(s) prevent the tail from unthreading from the ring. In some embodiments, amino acid residue(s) having sterically side chains are located and/or introduced to both the loop and the tail portions of the lasso peptide. In particular embodiments, a bulky amino acid residue in the loop portion is away from a bulky amino acid residue in the tail portion of the lasso peptide by at least 1 non-bulky amino acid residues. In particular embodiments, a bulky amino acid residue in the loop portion is away from a bulky amino acid residue in the tail portion of the lasso peptide by about 2, 3, 4, 5, or 6 non-bulky amino acid residues. In various embodiments, one or more sterically bulky amino acid residues as described herein are introduced to the nucleic acid sequence of a lasso peptide by amino acid addition or amino acid substitution mutations using the methods described herein and/or known in the art.

Various methods have been developed for mutagenesis of genes. A few examples of such mutagenesis methods are provided below. One or more of these methods can be used in connection with the present disclosure to produced diversified nucleic acids sequences coding for different lasso precursor peptides or lasso core peptides, which can be used to produce libraries of lasso peptides using the CFB methods and systems described herein.

Error-prone PCR, or epPCR (Pritchard, L., D. Come, D. Kell, J. Rowland, and M. Winson, 2005, A general model of error-prone PCR J Theor. Biol 234:497-509), introduces random point mutations by reducing the fidelity of DNA polymerise in PCR reactions by the addition of Mn²⁺ ions, by biasing dNTP concentrations, or by other conditional variations. The five step cloning process to confine the mutagenesis to the target gene of interest involves: 1) error-prone PCR amplification of the gene of interest; 2) restriction enzyme digestion; 3) gel purification of the desired DNA fragment; 4) ligation into a vector; 5) expression of the gene variants using a CFB system and screening of the library of expressed lasso peptides for improved performance. This method can generate multiple mutations in a single gene or coding sequence simultaneously, which can be useful. A high number of mutants can be generated by epPCR, so a high-throughput screening assay or a selection method (especially using robotics) is useful to identify those with desirable characteristics.

Error-prone Rolling Circle Amplification (epRCA) (Fujii, R, M. Kitaoka, and K. Hayashi, 2004, One-step random mutagenesis by error-prone rolling circle amplification. Nucleic Acids Res 32:e 145; and Fujii, R., M. Kitaoka, and K. Hayashi, 2006, Error-prone rolling circle amplification: the simplest random mutagenesis protocol. Nat. Protoc. 1:2493-2497.) has many of the same elements as epPCR except a whole circular plasmid is used as the template and random 6-mers with exonuclease resistant thiophosphate linkages on the last 2 nucleotides are used to amplify the plasmid followed by expression of the variants in a CFB system, in which the plasmid is re-circularized at tandem repeats. Adjusting the Mn²⁺ concentration can vary the mutation rate somewhat. This technique uses a simple error-prone, single-step method to create a full copy of the plasmid with 3-4 mutations/kbp. No restriction enzyme digestion or specific primers are required. Additionally, this method is typically available as a kit.

DNA or Family Shuffling (Stemmer, W. P. 1994, DNA shuffling by random fragmentation and reassembly: in vitro recombination for molecular evolution. Proc Natl Acad Sci US.A 91:10747-10751; and Stemmer, W. P. 1994. Rapid evolution of a protein in vitro by DNA shuffling Nature 370:389-391.) typically involves digestion of 2 or more variant genes or coding sequences with nucleases such as DNase I or EndoV to generate a pool of random fragments that are reassembled by cycles of annealing and extension in the presence of DNA polymerase to create a library of chimeric genes. Fragments prime each other and recombination occurs when one copy primes another copy (template switch). This method can be used with >1 kbp DNA sequences. In addition to mutational recombinants created by fragment reassembly, this method introduces point mutations in the extension steps at a rate similar to error-prone PCR.

Staggered Extension (StEP) (Zhao, H., L. Giver, Z. Shao, J. A. Affholter, and F. H. Arnold, 1998, Molecular evolution by staggered extension process (StEP) in vitro recombination. Nat. Biotechnol., 16:258-261.) entails template priming followed by repeated cycles of 2-step PCR with denaturation and very short duration of annealing/extension (as short as 5 sec). Growing fragments anneal to different templates and extend further, which is repeated until full-length sequences are made. Template switching means most resulting fragments have multiple parents. Combinations of low-fidelity polymerases (Taq and Mutazyme) reduce error-prone biases because of opposite mutational spectra.

In Random Priming Recombination (RPR) random sequence primers are used to generate many short DNA fragments complementary to different segments of the template. (Shao, Z., H. Zhao, L. Giver, and F. H. Arnold, 1998, Random-priming in vitro recombination: an effective tool for directed evolution. Nucleic Acids Res, 26:681-683.) Base misincorporation and mispriming via epPCR give point mutations. Short DNA fragments prime one another based on homology and are recombined and reassembled into full-length by repeated thermocycling. Removal of templates prior to this step assures low parental recombinants. This method, like most others, can be performed over multiple iterations to evolve distinct properties. This technology avoids sequence bias, is independent of gene length, and requires very little parent DNA for the application.

In Heteroduplex Recombination linearized plasmid DNA is used to form heteroduplexes that are repaired by mismatch repair. (Volkov, A. A., Z. Shao, and F. H. Arnold. 1999. Recombination and chimeragenesis by in vitro heteroduplex formation and in vivo repair. Nucleic Acids Res, 27:e18; and Volkov, A. A., Z. Shao, and F. H. Arnold. 2000. Random chimeragenesis by heteroduplex recombination. Methods Enzymol., 328:456-463.) The mismatch repair step is at least somewhat mutagenic. Heteroduplexes transform more efficiently than linear homoduplexes. This method is suitable for large genes and whole operons.

Random Chimeragenesis on Transient Templates (RACHTIT) (Coco, W. M., W. E. Levinson, M. J. Crist, H. J. Hektor, A. Darzins, P. T. Pienkos, C. H. Squires, and D. J. Monticello, 2001, DNA shuffling method for generating highly recombined genes and evolved enzymes. Nat. Biotechnol., 19:354-359.) employs DNase I fragmentation and size fractionation of ssDNA. Homologous fragments are hybridized in the absence of polymerase to a complementary ssDNA scaffold. Any overlapping unhybridized fragment ends are trimmed down by an exonuclease. Gaps between fragments are filled in, and then ligated to give a pool of full-length diverse strands hybridized to the scaffold (that contains U to preclude amplification). The scaffold then is destroyed and is replaced by a new strand complementary to the diverse strand by PCR amplification. The method involves one strand (scaffold) that is from only one parent while the priming fragments derive from other genes; the parent scaffold is selected against. Thus, no reannealing with parental fragments occurs. Overlapping fragments are trimmed with an exonuclease. Otherwise, this is conceptually similar to DNA shuffling and StEP. Therefore, there should be no siblings, few inactives, and no unshuffled parentals. This technique has advantages in that few or no parental genes are created and many more crossovers can result relative to standard DNA shuffling.

Recombined Extension on Truncated templates (RE 1′1) entails template switching of unidirectionally growing strands from primers in the presence of unidirectional ssDNA fragments used as a pool of templates. (Lee, S. H., E. J. Ryu, M. J. Kang, E.-S. Wang, Z. C. Y. Piao, K. J. J. Jung, and Y. Shin, 2003, A new approach to directed gene evolution by recombined extension on truncated templates (RETT). J. Molec. Catalysis 26:119-129.) No DNA endonucleases are used. Unidirectional ssDNA is made by DNA polymerase with random primers or serial deletion with exonuclease. Unidirectional ssDNA are only templates and not primers. Random priming and exonucleases don't introduce sequence bias as true of enzymatic cleavage of DNA shuffling/RACHTIT. RETT can be easier to optimize than StEP because it uses normal PCR conditions instead of very short extensions. Recombination occurs as a component of the PCR steps—no direct shuffling. This method can also be more random than StEP due to the absence of pauses.

In Degenerate Oligonucleotide Gene Shuffling (DOGS) degenerate primers are used to control recombination between molecules; (Bergquist, P. L. and M. D. Gibbs, 2007, Degenerate oligonucleotide gene shuffling Methods Mol. Biol., 352:191-204; Bergquist, P. L., R A. Reeves, and M. D. Gibbs, 2005, Degenerate oligonucleotide gene shuffling (DOGS) and random drift mutagenesis (RNDM): two complementary techniques for enzyme evolution. Biomol. Eng., 22:63-72; Gibbs, M. D., K. M. Nevalainen, and P. L. Bergquist, 2001, Degenerate oligonucleotide gene shuffling (DOGS): a method for enhancing the frequency of recombination with family shuffling Gene 271:13-20.) this can be used to control the tendency of other methods such as DNA shuffling to regenerate parental genes. This method can be combined with random mutagenesis (epPCR) of selected gene segments. This can be a good method to block the reformation of parental sequences. No endonucleases are needed. By adjusting input concentrations of segments made, one can bias towards a desired backbone. This method allows DNA shuffling from unrelated parents without restriction enzyme digests and allows a choice of random mutagenesis methods.

Incremental Truncation for the Creation of Hybrid Enzymes (ITCHY) creates a combinatorial library with 1 base pair deletions of a gene or gene fragment of interest. (Ostermeier et al., Proc. Natl. Acad. Sci. US.A. 96:3562-3567 (1999); Ostermeier et al., 1999 Nat. Biotechnol., 17:1205-1209 (1999)) Truncations are introduced in opposite direction on pieces of 2 different genes. These are ligated together and the fusions are cloned. This technique does not require homology between the 2 parental genes. When ITCHY is combined with DNA shuffling, the system is called SCRATCHY (see below). A major advantage of both is no need for homology between parental genes; for example, functional fusions between an E. coli and a human gene were created via ITCHY. When ITCHY libraries are made, all possible crossovers are captured.

Thio-Incremental Truncation for the Creation of Hybrid Enzymes (TRIO-ITCHY) is almost the same as ITCHY except that phosphothioate dNTPs are used to generate truncations. (Lutz, S., M. Ostermeier, and S. J. Benkovic, 2001, Rapid generation of incremental truncation libraries for protein engineering using alpha-phosphothioate nucleotides. Nucleic Acids Res 29:E16.) Relative to ITCHY, TRIO-ITCHY can be easier to optimize, provide more reproducibility, and adjustability.

SCRATCHY-ITCHY combined with DNA shuffling is a combination of DNA shuffling and ITCHY; therefore, allowing multiple crossovers. (Lutz et al., Proc. Natl. Acad. Sci. US.A. 98:11248-11253 (2001).) SCRATCHY combines the best features of ITCHY and DNA shuffling Computational predictions can be used in optimization. SCRATCHY is more effective than DNA shuffling when sequence identity is below 80%.

In Random Drift Mutagenesis (RNDM) mutations made via epPCR followed by screening/selection for those retaining usable activity. (Bergquist et al., Biomol. Eng., 22:63-72 (2005)) Then, these are used in DOGS to generate recombinants with fusions between multiple active mutants or between active mutants and some other desirable parent. Designed to promote isolation of neutral mutations; its purpose is to screen for retained catalytic activity whether or not this activity is higher or lower than in the original gene. RNDM is usable in high throughput assays when screening is capable of detecting activity above background. RNDM has been used as a front end to DOGS in generating diversity. The technique imposes a requirement for activity prior to shuffling or other subsequent steps; neutral drift libraries are indicated to result in higher/quicker improvements in activity from smaller libraries. Though published using epPCR, this could be applied to other large-scale mutagenesis methods.

Sequence Saturation Mutagenesis (SeSaM) is a random mutagenesis method that: 1) generates pool of random length fragments using random incorporation of a phosphothioate nucleotide and cleavage; this pool is used as a template to 2) extend in the presence of “universal” bases such as inosine; 3) replication of a inosine-containing complement gives random base incorporation and, consequently, mutagenesis. (Wong et al., Biotechnol J. 3:74-82 (2008); Wong Nucleic Acids Res 32:e26; Wong et al., Anal. Biochem., 341:187-189 (2005).) Using this technique it can be possible to generate a large library of mutants within 2-3 days using simple methods. This is very non-directed compared to mutational bias of DNA polymerases. Differences in this approach makes this technique complementary (or alternative) to epPCR

In Synthetic Shuffling, overlapping oligonucleotides are designed to encode “all genetic diversity in targets” and allow a very high diversity for the shuffled progeny. (Ness, et al., Nat. Biotechnol., 20:1251-1255 (2002)) In this technique, one can design the fragments to be shuffled. This aids in increasing the resulting diversity of the progeny. One can design sequence/codon biases to make more distantly related sequences recombine at rates approaching more closely related sequences and it doesn't require possessing the template genes physically.

Nucleotide Exchange and Excision Technology NexT exploits a combination of dUTP incorporation followed by treatment with uracil DNA glycosylase and then piperidine to perform endpoint DNA fragmentation. (Muller et al., Nucleic Acids Res 33:e117 (2005)) The gene is reassembled using internal PCR primer extension with proofreading polymerase. The sizes for shuffling are directly controllable using varying dUTP::dTTP ratios. This is an end point reaction using simple methods for uracil incorporation and cleavage. One can use other nucleotide analogs such as 8-oxo-guanine with this method. Additionally, the technique works well with very short fragments (86 bp) and has a low error rate. Chemical cleavage of DNA means very few unshuffled clones.

In Sequence Homology-Independent Protein Recombination (SHIPREC) a linker is used to facilitate fusion between 2 distantly/unrelated genes; nuclease treatment is used to generate a range of chimeras between the two. Result is a single crossover library of these fusions. (Sieber, V., C. A. Martinez, and F. H. Arnold. 2001. Libraries of hybrid proteins from distantly related sequences. Nat. Biotechnol., 19:456-460.) This produces a limited type of shuffling; mutagenesis is a separate process. This technique can create a library of chimeras with varying factions of each of 2 unrelated parent genes. No homology is needed. SHIPREC was tested with a heme-binding domain of a bacterial CP450 fused to N-terminal regions of a mammalian CP450; this produced mammalian activity in a more soluble enzyme.

Saturation mutagenesis is a random mutagenesis technique, in which a single codon or set of codons is randomised to produce all possible amino acids at the position. Saturation mutagenesis is commonly achieved by artificial gene synthesis, with a mixture of nucleotides used at the codons to be randomised. Different degenerate codons can be used to encode sets of amino acids. Because some amino acids are encoded by more codons than others, the exact ratio of amino acids cannot be equal. Additionally, it is usual to use degenerate codons that minimise stop codons (which are generally not desired). Consequently, the fully randomised ‘NNN’ is not ideal, and alternative, more restricted degenerate codons are used. ‘NNK’ and ‘NNS’ have the benefit of encoding all 20 amino acids, but still encode a stop codon 3% of the time. Alternative codons such as ‘NDT’, ‘DBK’ avoid stop codons entirely, and encode a minimal set of amino acids that still encompass all the main biophysical types (anionic, cationic, aliphatic hydrophobic, aromatic hydrophobic, hydrophilic, small).

Gene Reassembly is a DNA shuffling method that can be applied to multiple genes at one time or to creating a lie library of chimeras (multiple mutations) of a single gene. Typically this technology is used in combination with ultra-high-throughput screening to query the represented sequence space for desired improvements. This technique allows multiple gene recombination independent of homology. The exact number and position of cross-over events can be pre-determined using fragments designed via bioinformatic analysis. This technology leads to a very high level of diversity with virtually no parental gene reformation and a low level of inactive genes. Combined with GSSM, a large range of mutations can be tested for improved activity. The method allows “blending” and “fine tuning” of DNA shuffling, e.g. codon usage can be optimized.

In Gene Site Saturation Mutagenesis (GSSM) the starting materials are a supercoiled dsDNA plasmid with insert and 2 primers degenerate at the desired site for mutations. (Kretz, K. A., T. H. Richardson, K. A. Gray, D. E. Robertson, X. Tan, and J. M. Short, 2004, Gene site saturation mutagenesis: a comprehensive mutagenesis approach. Methods Enzymol., 388:3-11.) Primers carry the mutation of interest and anneal to the same sequence on opposite strands of DNA; mutation in the middle of the primer and ˜20 nucleotides of correct sequence flanking on each side. The sequence in the primer is NNN or NNK (coding) and MNN (noncoding) (N=all 4, K=G, T, M=A, C). After extension, DpnI is used to digest dam-methylated DNA to eliminate the wild-type template. This technique explores all possible amino acid substitutions at a given locus (i.e., one codon). The technique facilitates the generation of all possible replacements at one site with no nonsense codons and equal or near-equal representation of most possible alleles. It does not require prior knowledge of structure, mechanism, or domains of the target enzyme. If followed by shuffling or Gene Reassembly, this technology creates a diverse library of recombinants containing all possible combinations of single-site up-mutations. The utility of this technology combination has been demonstrated for the successful evolution of over 50 different enzymes, and also for more than one property in a given enzyme.

Combinatorial Cassette Mutagenesis (CCM) involves the use of short oligonucleotide cassettes to replace limited regions with a large number of possible amino acid sequence alterations. (Reidhaar-Olson, J. F., J. U. Bowie, R M. Breyer, J. C. Hu, K. L. Knight, W. A. Lim, M. C. Mossing, D. A. Parsell, K. R Shoemaker, and R T. Sauer, 1991, Random mutagenesis of protein sequences using oligonucleotide cassettes. Methods Enzymol., 208:564-586; and Reidhaar-Olson, J. F. and R T. Sauer, 1988, Combinatorial cassette mutagenesis as a probe of the informational content of protein sequences. Science 241:53-57.) Simultaneous substitutions at 2 or 3 sites are possible using this technique. Additionally, the method tests a large multiplicity of possible sequence changes at a limited range of sites. It has been used to explore the information content of lambda repressor DNA-binding domain.

Combinatorial Multiple Cassette Mutagenesis (CMCM) is essentially similar to CCM except it is employed as part of a larger program: 1) Use of epPCR at high mutation rate, 2) Identification of hot spots and hot regions and then 3) extension by CMCM to cover a defined region of protein sequence space. (Reetz, M. T., S. Wilensek, D. Zha, and K. E. Jaeger, 2001, Directed Evolution of an Enantioselective Enzyme through Combinatorial Multiple-Cassette Mutagenesis. Angew. Chem. Int. Ed Engl. 40:3589-3591.) As with CCM, this method can test virtually all possible alterations over a target region. If used along with methods to create random mutations and shuffled genes, it provides an excellent means of generating diverse, shuffled proteins. This approach was successful in increasing, by 51-fold, the enantioselectivity of an enzyme.

In the Mutator Strains technique conditional is mutator plasmids allow increases of 20- to 4000-X in random and natural mutation frequency during selection and to block accumulation of deleterious mutations when selection is not required. (Selifonova, O., F. Valle, and V. Schellenberger, 2001, Rapid evolution of novel traits in microorganisms. Appl Environ Microbiol., 67:3645-3649.) This technology is based on a plasmid-derived mutD5 gene, which encodes a mutant subunit of DNA polymerase III. This subunit binds to endogenous DNA polymerise III and compromises the proofreading ability of polymerise III in any of the strain that harbors the plasmid. A broad-spectrum of base substitutions and frameshift mutations occur. In order for effective use, the mutator plasmid should be removed once the desired phenotype is achieved; this is accomplished through a temperature sensitive origin of replication, which allows plasmid curing at 41° C. It should be noted that mutator strains have been explored for quite some time (e.g., see Winter and coworkers, 1996, J. Mol. Biol. 260, 359-3680. In this technique very high spontaneous mutation rates are observed. The conditional property minimizes non-desired background mutations. This technology could be combined with adaptive evolution to enhance mutagenesis rates and more rapidly achieve desired phenotypes.

“Look-Through Mutagenesis (LTM) is a multidimensional mutagenesis method that assesses and optimizes combinatorial mutations of selected amino acids.” (Rajpal, A., N. Beyaz, L. Haber, G. Cappuccilli, H. Yee, R R Bhatt, T. Takeuchi, R A. Lerner, and R Crea, 2005, A general method for greatly improving the affinity of antibodies by using combinatorial libraries. Proc. Natl. Acad. Sci. USA., 102:8466-8471.) Rather than saturating each site with all possible amino acid changes, a set of 9 is chosen to cover the range of amino acid R group chemistry. Fewer changes per site allows multiple sites to be subjected to this type of mutagenesis. A >800-fold increase in binding affinity for an antibody from low nanomolar to picomolar has been achieved through this method. This is a rational approach to minimize the number of random combinations and should increase the ability to find improved traits by greatly decreasing the numbers of clones to be screened. This has been applied to antibody engineering, specifically to increase the binding affinity and/or reduce dissociation. The technique can be combined with either screens or selections.

In Silico Protein Design Automation PDA is an optimization algorithm that anchors the structurally defined protein backbone possessing a particular fold, and searches sequence space for amino acid substitutions that can stabilize the fold and overall protein energetics. (Hayes, R. J., J. Bentzien, M. L. Ary, M. Y. Hwang, J. M. Jacinto, J. Vielmetter, A. Kundu, and B. I. Dahiyat, 2002, Combining computational and experimental screening for rapid optimization of protein properties. Proc. Natl. Acad. Sci. USA., 99:15926-15931.) This technology allows in silico structure-based entropy predictions in order to search for structural tolerance toward protein amino acid variations. Statistical mechanics is applied to calculate coupling interactions at each position—structural tolerance toward amino acid substitution is a measure of coupling. Ultimately, this technology is designed to yield desired modifications of protein properties while maintaining the integrity of structural characteristics. The method computationally assesses and allows filtering of a very large number of possible sequence variants (10⁵⁰). Choice of sequence variants to test is related to predictions based on most favorable thermodynamics and ostensibly only stability or properties that are linked to stability can be effectively addressed with this technology. The method has been successfully used in some therapeutic proteins, especially in engineering immunoglobulins. In silico predictions avoid testing extraordinarily large numbers of potential variants. Predictions based on existing three-dimensional structures are more likely to succeed than predictions based on hypothetical structures. This technology can readily predict and allow targeted screening of multiple simultaneous mutations, something not possible with purely experimental technologies due to exponential increases in numbers.

Iterative Saturation Mutagenesis (ISM) involves: (1) use knowledge of structure/function to choose a likely site for enzyme improvement, (2) saturation mutagenesis at the chosen site using Agilent QuickChange (or other suitable means), (3) screen/select for desired properties, (4) with improved clone(s), start over at another site and continue repeating. (Reetz, M. T. and J. D. Carballeira, 2007, Iterative saturation mutagenesis (ISM) for rapid directed evolution of functional enzymes. Nat. Protoc. 2:891-903; and Reetz, M. T., J. D. Carballeira, and A. Vogel, 2006, Iterative saturation mutagenesis on the basis of B factors as a strategy for increasing protein thermos stability. Angew. Chem. Int. Ed Engl. 45:7745-7751.) This is a proven methodology assures all possible replacements at a given position are made for screening/selection.

Any of the aforementioned methods for mutagenesis can be used alone or in any combination. Additionally, any one or combination of the directed evolution methods can be used in conjunction with adaptive evolution techniques.

Additional diversification of a lasso peptide library can be achieved via chemical or enzymatic modifications. In specific embodiments, the lasso peptide component is further modified chemically or enzymatically. Particularly, in some embodiments, enzyme modifications of the lasso peptide component comprises modification by halogenation, lipidation, pegylation, glycosylation, adding hydrophobic groups, myristoylation, palmitoylation, isoprenylation, prenylation, lipoylation, adding a flavin moiety (optionally comprising addition of a flavin adenine dinucleotide (FAD) an FADH₂, a flavin mononucleotide (FMN), an FMNH₂), phospho-pantetheinylation, heme C addition, phosphorylation, acylation, alkylation, butyrylation, carboxylation, malonylation, hydroxylation, adding a halide group, iodination, propionylation, S-glutathionylation, succinylation, glycation, adenylation, thiolation, condensation. Particularly, in some embodiments condensation comprises addition of an amino acid to an amino acid, an amino acid to a fatty acid, or an amino acid to a sugar. In some embodiments, enzymatic modification of the lasso peptide component comprises a combination of one or more aforementioned modifications. For example, in some embodiments, enzyme modification comprises modification of the lasso peptide component by one or more enzymes selected from a CoA ligase, a phosphorylase, a kinase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof. In some embodiments, the enzymes comprise one or more central metabolism enzyme (e.g., tricarboxylic acid cycle (TCA, or Krebs cycle) enzymes, glycolysis enzymes or Pentose Phosphate Pathway enzymes). In some embodiments, chemical or enzyme modifications to the lasso peptide component comprise addition, deletion or replacement of a substituent or functional groups, e.g., a hydroxyl group, an amino group, a halogen, an alkyl or a cycloalkyl group, or by hydration, biotinylation, hydrogenation, an aldol condensation reaction, condensation polymerization, halogenation, oxidation, dehydrogenation, or creating one or more double bonds.

In some embodiments, the diversified species of lasso peptides are screened for one or more desirable target properties, and one or more lasso peptides are further selected to serve as the new scaffold for at least one additional round of mutagenesis and screening.

5.3.6.3 Phage Production by Host Organisms

As described herein, the nucleic acids and systems of nucleic acids for producing one or more lasso-displaying phage as described herein (e.g., in above sections titled ‘Nucleic Acid’ and ‘System for Producing Phage Display Libraries’) can be introduced into a suitable host cell, which host cell can then be cultured under a suitable condition to produce the phages. In some embodiments, the host organism can be used to produce either a population of phages displaying the same lasso peptide component, or a library comprising a plurality of phages displaying diversified lasso peptide components. Particularly, to produce the phage display library, one or more nucleic acid sequences encoding the displayed lasso peptide components can be diversified as described herein (e.g., in above section titled ‘Diversifying Lasso Peptides’) before introducing into the host organism. Further, a nucleic acid sequence encoding a displayed lasso peptide component can be introduced into the host organism in combination with different nucleic acid sequences encoding the lasso peptide biosynthesis component to further diversify the library as described herein (e.g., in above section titled ‘Diversifying Lasso Peptides’).

In some embodiments, the host organisms for producing the lasso-displaying phages is a bacteria. In some embodiments, the host organism for producing the lasso-displaying phages is an archaea. In some embodiments, the host is a bacteria susceptible to phage infection. In some embodiments, the host is a Gram-negative bacteria. In some embodiments, the host is a Gram-positive bacteria. In some embodiments, the host is an archaea susceptible to phage infection. In some embodiments, the host is susceptible to infection by a budding phage. In some embodiments, the host is susceptible to infection by a lytic phage. In some embodiments, the host is E. coli.

In some embodiments, the host microorganism is genetically engineered to express a protein that contain at least one non-natural or unusual amino acid residues. For example, Wals et al. “Unnatural amino acid incorporation in E. coli: current and future applications in the design of therapeutic proteins” Front Chem. 2014 Apr. 1; 2:15 describes genetically modified E. coli expression systems capable of incorporating unnatural or unusual amino acid residues into protein products.

In some embodiments, the such expression system uses amber codon suppression. This technology allows the incorporation of a single UAA at a specific site in a protein using a tRNA that recognizes an amber codon (TAG in DNA, UAG in mRNA, and CUA in tRNA). Amber codon suppression involves the following components: mRNA containing the amber codon at the position to incorporate a UAA, modified aminoacyl-tRNA synthetase (aaRS) that is capable of recognizing the UAA, and complementary tRNA (amber tRNA^{CUA}) that can be aminoacylated by the modified aaRS. To incorporate a UAA, the modified aaRS is orthogonal to the tRNA^{CUA} loading machinery of the expression host to allow loading of the UAA onto the tRNA^{CUA}. The tRNA^{CUA} then recognizes the amber codon in the mRNA, resulting in protein with incorporated UAA at a specific site.

Another exemplary host expression system that is genetically modified for incorporating UAAs into protein products uses four-base codon suppression. Four-base codon can encode multiple distinct UAA into protein and requires aaRS and tRNA pairs that can decode the four-base codons. For example, Hohsaka et al. used four-base codons, such as AGGU and CGGG, together in a single transcript and inserted two different UAAs into the same protein site-specifically (Hohsaka et al., J. Am. Chem. Soc., 1999, 121, 12194-12195).

It is also possible to combine UAA incorporation with library-based screening procedures of protein or polypeptides for a desirable target property (Wals et al. Supra.). Specifically, screening can possibly be carried out by combination of three libraries in the host, such as E coli, namely an aaRS mutant and tRNA mutant library, a protein or peptide mutant library, and a UAA library. For example, the three libraries described above can be co-transformed into E. coli to produce mutant proteins or polypeptides and to select or screen them for a desirable target property using proper screening procedures.

In some embodiments, the genetically engineered E. coli cell comprises a nucleic acid sequence encoding a modified aminoacyl-tRNA synthetase (aaRS) capable of recognizing an unusual or unnatural amino acid. In some embodiments, the nucleic acid sequence further encode a complementary tRNA that can be aminoacylated by the modified aaRS. In some embodiments, the genetically engineered E. coli cell comprises a complementary tRNA (e.g., amber tRNA^{CUA}) that can be aminoacylated by the modified aaRS. In some embodiments, the complementary tRNA can be selected from an amber tRNA^{CUA} and a tRNA decodes a four-base codon. In some embodiments, the genetically engineered host cell comprises a mRNA that contains the amber codon UAG. In some embodiments, the genetically engineered host cell comprises a mRNA that contains a four-base codon. In some embodiments, the host microorganism is cultured in a medium comprising at least one unnatural or unusual amino acid. In some embodiments, the UAA incorporation and screen of a phage display lasso peptide library can be carried out at the same time. In some embodiments, the UAA incorporation uses amber codon suppression and/or four-base codon suppression. In some embodiment, a phage display lasso peptide library, an aaRS and tRNA library, and a UAA library can be co-transformed into a host to produce and screen mutant lasso peptides having incorporated UAAs and a desirable target property.

In some embodiments, the UAA incorporated in the produced protein product can be utilized to introduce post-translational modifications, such as lysine methylation (Nguyen et al. J. Am. Chem. Soc., 2009, 131, 14194-14195), acetylation (Neumann et al., Mol. Cell, 2009, 36, 153-163), and ubiquitination (Virdee et al., Nat. Chem. Biol., 2010, 6, 750-757).

In some embodiments, the host microorganism is genetically engineered to introduce one or more non-natural post-translational modifications to an expressed protein product, such as glycosylation, lysine methylation (Nguyen et al. J. Am. Chem. Soc., 2009, 131, 14194-14195), acetylation (Neumann et al., Mol. Cell, 2009, 36, 153-163), and ubiquitination (Virdee et al., Nat. Chem. Biol., 2010, 6, 750-757). For example, E coli. strains that are developed by transplanting and adapting the N-glycosylation system found in Campylobacter jejuni can be used to introduce glycosylation to an expressed protein product (Wacker et al., Science, 2002, 298, 1790-1793). Eukaryotic host Pichia pastoris can be modified to produce antibodies with specific human N-glycan structure (Li et al., Nat. Biotechnol., 2006, 24, 210-215). Furthermore, to obtain correct disulfide formation in the production of proinsulin, a therapeutic protein that containing 3 disulfide bridges, Rudolph et al. used a fusion of pro-insulin to the periplasmic E. coli protein disulfide oxidoreductase (DsbA). In some embodiments, the host microorganism is genetically engineered to introduce one or more non-natural post-translational modifications to lasso peptides produced. The post-translational modifications include, but are not limited to, glycosylation, lysine methylation, acetylation, and ubiquitination.

Metabolic modeling and simulation algorithms can be utilized. Modeling can also be used to design gene knockouts that additionally optimize utilization of the lasso peptide pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on shifting the primary metabolism towards more efficient production of exogenously encoded lasso peptide component, lasso peptide biosynthesis component, and phage proteins by the host cells.

One computational method for identifying and designing metabolic alterations favoring biosynthesis of a desired product is the OptKnock computational framework (Burgard et al., Biotechnol. Bioeng., 2003, 84, 647-657). OptKnock is a metabolic modeling and simulation program that suggests gene deletion or disruption strategies that result in genetically stable metabolic network which overproduces the target product. Specifically, the framework examines the complete metabolic and/or biochemical network in order to suggest genetic manipulations that lead to maximum production of a lasso peptide or related molecules thereof. Such genetic manipulations can be performed on strains used to produce cell lines optimized for the exogenously encoded proteins described herein. Also, this computational methodology can be used to either identify alternative pathways that lead to biosynthesis of a desired lasso peptide or used in connection with non-naturally occurring systems for further optimization of biosynthesis of a lasso peptide.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which biosynthetic performance can be predicted.

Given the teachings and guidance provided herein, those skilled in the art will be able to apply various computational frameworks for metabolic modeling and simulation to design and implement biosynthesis of exogenously encoded protein components in the host cell. Such metabolic modeling and simulation methods include, for example, the computational systems exemplified above as SimPheny® and OptKnock. Those skilled in the art will know how to apply the identification, design and implementation of the metabolic alterations using OptKnock to any of such other metabolic modeling and simulation computational frameworks and methods well known in the art.

Methods for constructing and testing the levels expression of exogenously encoded proteins and production of lasso-presenting phages by the host microorganism can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences encoding the phage component, lasso peptide component or lasso peptide biosynthesis component as described herein can be introduced stably or transiently into a host cell using techniques well known in the art including, but not limited to, conjugation, electroporation, chemical transformation, transduction, transfection, and ultrasound transformation. One or more exogenous nucleic acid sequences can be included in the genome of an infectious phage, and introduced into the host cell through infection of the host cell by the phage.

For exogenous expression in E. coli or other prokaryotic cells, some nucleic acid sequences in the genes or cDNAs of eukaryotic nucleic acids can encode targeting signals such as an N-terminal mitochondria) or other targeting signal, which can be removed before transformation into prokaryotic host cells, if desired. For example, removal of a mitochondria) leader sequence led to increased expression in E. coli (Hofliueister et al., J. Biol. Chem. 280:4329-4338 (2005)). Genes can be expressed in the cytosol without the addition of leader sequence, or can be targeted to an organelle, or periplasmic space, or targeted for secretion, by the addition of a suitable targeting sequence such as a periplasmic targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more encoding nucleic acid sequences as exemplified herein operably linked to expression control sequences functional in the host organism. Expression vectors applicable for use in the microbial host organisms of the invention include, for example, plasmids, phage vectors (e.g. phagemid), viral vectors, episomes and artificial chromosomes, including vectors and selection sequences or markers operable for stable integration into a host chromosome. Particularly, a particularly embodiment of an expression vector is a phagemid, comprising both a replication origin for duplicating the double-stranded sequence in the host microorganism, and a phage replication origin for duplicating the single-stranded sequence and packaging the single-stranded sequence into a phage capsid.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences encoding the phage component, lasso peptide component or lasso peptide biosynthesis component can be confirmed using methods well known in the art. Such methods include, for example, nucleic acid analysis such as Northern blots or polymerise chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

Suitable purification and/or assays to test for the production of the encoded proteins can be performed using well known methods. The individual enzyme or protein activities from the exogenous nucleic acid sequences can also be assayed using methods well known in the art (see, for example, WO/2008/115840 and Hanai et al., Appl. Environ. Microbiol. 73:7814-7818 (2007)).

The host microorganisms can be cultured in a medium with carbon source and other essential nutrients to grow and produce lasso-displaying phages. For certain host organisms, culturing can be maintained under anaerobic conditions. Such conditions can be obtained, for example, by first spying the medium with nitrogen and then sealing the flasks with a septum and crimp-cap. For host organisms where growth is not observed anaerobically, microaerobic conditions can be applied by perforating the septum with a small hole for limited aeration. Exemplary anaerobic conditions have been described previously and are well-known in the art. Exemplary aerobic and anaerobic conditions are described, for example, in United States Publication No. US-2009-0047719, filed Aug. 10, 2007.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

Host organisms of the present invention can utilize, for example, any carbohydrate source which can supply a source of carbon to the non-naturally occurring microorganism. Such sources include, for example, sugars such as glucose, xylose, arabinose, galactose, mannose, fructose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention.

Suitable purification and/or assays to test the production of phages can be performed using well known methods. For example, the phages can be separated from host cells or cell debris by centrifugation at a suitable speed. The phages can be harvested from supernatants while the host cell components are pelleted and discarded. The harvested phages can be subjected to one or more rounds of washing using a suitable buffer. Yield of the phage can be determined by UV absorbance as described by Day and Wiseman (The Single-Stranded DNA Phages, Cold Spring Harbor, N.Y., 1978, p 605): phage concentration (phages/mL)=((A₂₆₉−A₃₂₀)×6×10¹⁶)/(phage genome size in nt)×dilution factor, or the plaque assay, for lytic phages, as described by Jiang et al., Infect Immun. 1997, 65(11):4770-7.

Display of the lasso peptide component on the phage can be detected using methods known in the art. For example, a specific peptidase can be added to the harvested phage to cleave the peptidic linker between the lasso peptide component and the phage coat protein. The protease digestion reaction mixture is then centrifuged to precipitate insoluble debris. The soluble faction which contains released lasso peptide component can be then subjected to analysis using methods known in the art. For example, suitable replicates such as triplicate of the soluble faction, can be collected and analyzed to verify lasso peptide production and concentrations. The final concentrations of lasso peptide components can be analyzed by methods such as HPLC (High Performance Liquid Chromatography), GC-MS (Gas Chromatography-Mass Spectrometry), LC-MS (Liquid Chromatography-Mass Spectrometry), MALDI or other suitable analytical methods using routine procedures well known in the art. The presence of the phage nucleic acid sequences encoding the lasso peptide component in the pelleted phage-containing faction can be independently detected by PCR amplification and nucleic acid sequencing.

Lasso peptide components released from the phage can be isolated, separated purified using a variety of methods well known in the art. Such separation methods include, for example, extraction procedures, including using organic solvents such as methanol, butanol, ethyl acetate, and the like, as well as methods that include continuous liquid-liquid extraction, solid-liquid extraction, solid phase extraction, pervaporation, membrane filtration, membrane separation, reverse osmosis, electrodialysis, dialysis, distillation, crystallization, centrifugation, extractive filtration, ion exchange chromatography, size exclusion chromatography, adsorption chromatography, ultrafiltration, medium pressure liquid chromatograpy (MPLC), and high pressure liquid chromatography (HPLC). Additional separation and analytical methods suitable for recombinant proteins, such as affinity chromatography and ELISA can be used. All of the above methods are well known in the art and can be implemented in either analytical or preparative modes.

In some embodiments, a harvested phage population displaying the same lasso peptide component are placed in a separate location on a solid support, to be distinguished from another phage population displaying a different lasso peptide component. In other embodiments, a phage population displaying diversified lasso peptide components are mixed together in a library.

### 5.4 Screening and Evolution

The lasso peptides and functional fragments of lasso peptides provided herein can find uses in various aspects, including but are not limited to, diagnostic uses, prognostic uses, therapeutic uses, or as nutraceuticals or food supplements, for humans and animals. In some embodiments, the phage display libraries provided herein can be screened for members having one or more desirable properties, for example, by subjecting the library to various biological assays. In some embodiments, the library can be screened using assays known in the art.

According to the present disclosure, phage display library can be used in directed evolution of candidate lasso peptides for the generation of improved lasso peptides having those target properties. In some embodiments, the phage display library used in evolution can be produced using the methods described herein or any other methods.

Characteristics of lasso peptides that can be target properties include, for example, binding selectivity or specificity—for target-specific effects and avoiding off-target side effects or toxicity; binding affinity—for target-modulating potency and duration; temperature stability—for robust high temperature processing; pH stability—for bioprocessing under lower or higher pH conditions; expression level—increased protein yields. Other desirable target properties include, for example, solubility, metabolic stability, bioavailability, and pharmacokinetics. The present methods thus enable the discovery and optimization of lasso peptides and related molecules thereof for use in pharmaceutical, agricultural, and consumer applications.

Evolution of lasso peptide of interest using phage display library can be accomplished by various techniques known in the art. For example, a target molecule (e.g., a glucagon receptor (GCGR) polypeptide or fragment) can be used to coat the wells of adsorption plates, expressed on host cells affixed to adsorption plates or used in cell sorting, conjugated to biotin for capture with streptavidin-coated beads, or used in any other method for panning display libraries. The selection of lasso peptides with slow dissociation kinetics (e.g., good binding affinities) can be promoted by use of long washes and stringent panning conditions as described in Bass et al., 1990, Proteins 8:309-14 and WO 92/09690, and by use of a low coating density of target molecules as described in Marks et al., 1992, Biotechnol. 10:779-83.

Lasso peptides having one or more desirable target property(ies) can be obtained by designing a suitable screening procedure to select for one or more candidate members from the phage-displayed lasso peptide library as scaffold(s), followed by evolving the scaffolds towards improved target property.

5.4.1 Screening Lasso Peptides for Desirable Target Properties Using a Phage Display Library

Provided herein are phage display libraries that comprise lasso peptide components. In various embodiments, the lasso peptide component can assume the form of (i) an intact lasso peptide, (ii) a functional fragment of a lasso peptide, (iii) a lasso precursor peptide, or (iv) a lasso core peptide. In particular embodiments, the phage displayed lasso peptide component is lasso peptides having the lariat-like topology. In particular embodiments, the phage displayed lasso peptide component is a function fragment of a lasso peptide as described herein. In some embodiments, neither the non-lasso component of the coat protein nor other components of the phage interferes with either the functional or structural feature of the lasso peptide component.

A phage display library that comprises lasso peptide components can be screened for one or more target properties. In some embodiments, the phage display library is screened for library member(s) that shows affinity to a target molecule. In some embodiments, the phage display library is screened for library member(s) that specifically binds to a target molecule. In some embodiments, the phage display library is screened for library member(s) that specifically binds to a target site within a target molecule that has multiple sites capable of being bound by a ligand. In some embodiments, the phage display library is screened for library member(s) that compete for binding with a known ligand to a target molecule. In specific embodiments, such known ligand can also be a lasso peptide. In other embodiments, such known molecule can be a non-lasso ligand of the target molecule, such as a drug compound or a non-lasso protein. Various binding assays have been developed for testing the binding activity of members of a lasso peptide display library to a target molecule.

In one aspect, provided herein are methods for identifying a lasso peptide that specifically binds to a target molecule. In some embodiment, the method comprises providing a phage display library comprising a plurality of members, each member comprising a lasso peptide or a functional fragment of lasso peptide; contacting the library with the target molecule under a suitable condition that allows at least one member of the library to form a complex with the target molecule; and identifying the member of in the complex. In some embodiment, the contacting is performed by contacting the library with the target molecule in the presence of a reference binding partner of the target molecule under a suitable condition that allows at least one member of the library to compete with the reference binding partner for binding to the target molecule. In some embodiment, the identifying step is performed by detecting reduced binding of the reference binding partner to the target molecule; and identifying the member responsible for the reduced binding. In some embodiments, the reference binding partner is a ligand for the target molecule. In some embodiments, the target molecule comprises one or more target sites, and the reference binding partner specifically binds to a target site of the target molecule. In some embodiments, the reference binding partner is a natural ligand or synthetic ligand for the target molecule. In some embodiments, the target molecule is at least two target molecules.

Various binding assays can be used in connection with the present disclosure include immunoassays (e.g., ELISA, fluorescent immunosorbent assay, chemiluminescence immune assay, radioimmunoassay (RIA), enzyme multiplied immunoassay, solid phase radioimmunoassay (SPRIA)), a surface plasmon resonance (SPR) assay (e.g., Biacore®), a fluorescence polarization assay, a fluorescent resonance energy transfer (FRET) assay, Dot-blot assay, fluorescence activated cell sorting (FACS) assay. Depending on the target cellular activity of interest, those of ordinary skill in the art knows how to select a suitable binding assay for the screening.

In some embodiments, to identify a lasso peptide that modulates a cellular activity, a phage display library comprising lasso peptide components is screened for library members(s) that is capable of modulating one or more cellular activities. In some embodiments, a phage display library is subjected to a suitable biological assay that monitors the level of a cellular activity of interest. When a change in the level of the cellular activity of interest is detected, the member responsible for the detected change can be identified. In some embodiments, the library is subject to multiple biological assays configured for measuring the cellular activity; and the method further comprises selecting the members that have a high probability of being identified as responsible for the detected change in the cellular activity.

In some embodiments, the target molecule is a cell surface protein. In some embodiments, the phage display library comprising lasso peptide components is screened for library members(s) that is capable of modulating one or more cellular activities mediated by the cell surface protein. In some embodiments, a phage display library is subjected to a suitable biological assay that monitors the level of a cellular activity of interest, after the library is contacted with a cell expressing the target molecule. In some embodiments, a phage display library is subjected to a suitable biological assay that monitors a phenotype of interest of a cell after the library is contacted with a cell expressing the target molecule. In some embodiments, the target molecule is an unidentified cell surface protein expressed by a cell of interest. In some embodiments, a phage display library is subjected to a biological assay that monitors the level of a cellular activity of interest, after the library is contacted with a population of the cells of interest. In some embodiments, library member(s) that causes and/or enhances a cellular activity and/or cell phenotype of interest is selected. In other embodiments, library member(s) of that reduces and/or prevents a cellular activity and/or cell phenotype of interest is selected. Additionally or alternatively, in some embodiments, a phage display library is subjected to a biological assay that monitors a phenotype of the cell of interest, after the library is contacted with the cell.

In some embodiments, a phage display library is subjected to biological assays that monitor multiple related cellular activities. For example, in some embodiments, each of the multiple related cellular activities induces or inhibits the same cellular signaling pathway. In some embodiments, the multiple related cellular activities are implicated in the same pathological process. In some embodiments, the multiple related cellular activities are implicated in regulating the cell cycle. In some embodiments, each of the multiple related cellular activities induces or inhibits cell proliferation. In some embodiments, each of the multiple related cellular activities induces or inhibits cell differentiation. In some embodiments, each of the multiple related cellular activities induces or inhibits cell apoptosis. In some embodiments, each of the multiple related cellular activities induces or inhibits cell migration.

In some embodiments, to identify an agonist or antagonist lasso peptide for a target molecule, a phage display library comprising lasso peptide components is screened for library members(s) that is capable of binding to the target molecule. In some embodiments, a phage display library is contacted with a cell expressing the target molecule under a suitable condition that allows at least one member of the library to bind to the target molecule, and a cellular activity mediated by the target molecule is measured. In some embodiments, the cellular activity can be increased, and the member can be identified as an agonist ligand for the target molecule. In other embodiments, the cellular activity can be decreased, and the member can be identified as an antagonist ligand for the target molecule.

In some embodiments, library member(s) identified as responsible for a detected change in at least one monitored cellular activity is selected. In some embodiments, library member(s) identified as responsible for a detected change in at least two monitored cellular activities is selected. In some embodiments, library member(s) identified as responsible for a detected change in at least three monitored cellular activities is selected. In some embodiments, library member(s) identified as responsible for a detected change in at least 10% monitored cellular activities is selected. In some embodiments, library member(s) identified as responsible for a detected change in at least 20% monitored cellular activities is selected. In some embodiments, library member(s) identified as responsible for a detected change in at least 30% monitored cellular activities is selected. In some embodiments, library member(s) identified as responsible for a detected change in at least 40% monitored cellular activities is selected. In some embodiments, library member(s) identified as responsible for a detected change in at least 50% monitored cellular activities is selected. In some embodiments, library member(s) identified as responsible for a detected change in at least 60% monitored cellular activities is selected. In some embodiments, library member(s) identified as responsible for a detected change in at least 70% monitored cellular activities is selected. In some embodiments, library member(s) identified as responsible for a detected change in at least 80% monitored cellular activities is selected. In some embodiments, library member(s) identified as responsible for a detected change in at least 90% monitored cellular activities is selected.

In some embodiments, members of a first phage display library selected during a first round of screening for a first desirable property are assembled to into a second phage display library, and the second phage display library has an enriched population of members having the first desirable property. In some embodiments, the second phage display library is further subjected to a second round of screening for a second desirable property, and the selected library members are assembled into a third phage display library. The screening and selection processes can be repeated multiple times to produce one or more final selected member. In various embodiments, the first desirable property is the same as the second desirable property, and/or desirable property(ies) screened for in further round(s) of screens. In alternative embodiments, the first desirable property is different from the second desirable property, and/or desirable property(ies) screened for in further round(s) of screens. In some embodiments, the same desirable property is screened for under different conditions during the first and the second, or further round(s) of screens. For example, in specific embodiments, the desirable property is binding specificity of candidate library members to a target molecule, and during the sequential rounds of screens, the phage display library is subjected to more and more stringent conditions for the library members to bind to the target molecule. For example, in specific embodiments, the first desirable property is a high binding affinity (e g, binding affinity above a certain threshold value) of the candidate library members to a cell surface molecule, and the second desirable property is the ability of the candidate library members to enhance cell apoptosis mediated by the cell surface molecule.

In some embodiments, any method for screening for a desired enzyme activity, e.g., production of a desired product, e.g., such as a lasso peptide or related molecule thereof, can be used. Any method for isolating enzyme products or final products, e.g., lasso peptides or related molecules thereof, can be used. In alternative embodiments, methods and compositions of the present disclosure comprise use of any method or apparatus to detect a purposefully biosynthesized organic product, e.g., lasso peptide or related molecule thereof, or supplemented or microbially-produced organic products (e.g., amino acids, CoA, ATP, carbon dioxide), by e.g., employing invasive sampling of either cell extract or headspace followed by subjecting the sample to gas chromatography or liquid chromatography often coupled with mass spectrometry.

5.4.2 Directed Evolving of Lasso Peptides Using a Phage Display Library

Provided herein are phage display libraries that comprise lasso peptide components. In various embodiments, the lasso peptide component can assume the form of (i) an intact lasso peptide, (ii) a functional fragment of a lasso peptide, (iii) a lasso precursor peptide, or (iv) a lasso core peptide. In particular embodiments, the phage displayed lasso peptide component is lasso peptides having the lariat-like topology. In particular embodiments, the phage displayed lasso peptide component is a function fragment of a lasso peptide as described herein. In some embodiments, neither the non-lasso component of the coat protein nor other components of the phage interferes with either the functional or structural feature of the lasso peptide component.

Directed evolution is a powerful approach that involves the introduction of mutations targeted to a specific gene or an oligonucleotide sequence containing a gene in order to improve and/or alter the properties or production of an enzyme, protein or peptide (e.g., a lasso peptide). Improved and/or altered enzymes, proteins or peptides can be identified through the development and implementation of sensitive high-throughput assays that allow automated screening of many enzyme or peptide variants (for example, >10⁴). Iterative rounds of mutagenesis and screening typically are performed to afford an enzyme or peptide with optimized properties.

Computational algorithms that can help to identify areas of the gene for mutagenesis also have been developed and can significantly reduce the number of enzyme or peptide variants that need to be generated and screened (See: Fox, R J., et al., Trends Biotechnol., 2008, 26, 132-138; Fox, R J., et al., Nature Biotechnol., 2007, 25, 338-344). Numerous directed evolution technologies have been developed and shown to be effective at creating diverse variant libraries, and these methods have been successfully applied to the improvement of a wide range of properties across many enzyme and protein classes (for reviews, see: Hibbert et al., Biomol. Eng., 2005, 22, 11-19; Huisman and Lalonde, In Biocatalysis in the pharmaceutical and biotechnology industries, pgs. 717-742 (2007), Patel (ed.), CRC Press; Otten and Quax, Biomol. Eng., 2005, 22, 1-9; and Sen et al., Appl. Biochem. Biotechnol., 2007, 143, 212-223). Enzyme and protein characteristics that have been improved and/or altered by directed evolution technologies include, for example: selectivity/specificity, for conversion of non-natural substrates, temperature stability, for robust high temperature processing; pH stability, for bioprocessing under lower or higher pH conditions; substrate or product tolerance, so that high product titers can be achieved; binding (Kₘ), including broadening of ligand or substrate binding to include non-natural substrates, inhibition (Kᵢ), to remove inhibition by products, substrates, or key intermediates; activity (kₘ), to increase enzymatic reaction rates to achieve desired flux; isoelectric point (p1) to improve protein or peptide solubility; acid dissociation (pKₐ) to vary the ionization state of the protein or peptide with respect to pH; expression levels, to increase protein or peptide yields and overall pathway flux; oxygen stability, for operation of air-sensitive enzymes or peptides under aerobic conditions; and anaerobic activity, for operation of an aerobic enzyme or peptide in the absence of oxygen.

In one embodiment, a lasso peptide of interest is selected as the initial scaffold for directed evolution. Random mutations are introduced to a nucleic acid sequence encoding the initial scaffold, thereby producing a plurality of different mutated versions of the coding nucleic acid sequence. In some embodiments, a coding sequence of lasso precursor or lasso core peptide is mutated using the methods described herein or known in the art to produce a plurality of mutated versions of the coding sequence. Particularly, in some embodiments, the initial scaffold sequence is mutated by replacing one codon with a randomized codon (e.g., NNN) or a degenerated codon (e.g., NNK). In some embodiments such as those exemplified in Example 6, a plurality of initial scaffold sequences are individually mutated such that each mutated sequence has one codon replaced with a randomized or degenerated codon, and the replaced codons in the plurality of mutated sequences are each different from one another. In some embodiments such as those exemplified in Example 7, the initial scaffold sequence encoding a lasso core peptide is mutated by replacing all codons except the one coding for the ring-forming amino acid with a randomized or degenerated codon. In particular embodiments, the non-mutated codon encodes a glutamate residue (Glu) at the 7^{th}, 8^{th} or 9^{th} position counting from the N terminus of the encoded lasso core peptide. In particular embodiments, the non-mutated codon encodes an aspartate residue (Asp) at the 7^{th}, 8^{th} or 9^{th} position counting from the N terminus of the encoded lasso core peptide.

The plurality of mutated versions of the coding sequence are then used to produce a first phage display library comprising a plurality of members displaying distinct lasso peptides or functional fragments of lasso peptides using, for example, the methods disclosed herein. The library is then screened for candidate members having a desirable target property. Sequences of library members selected during the screen are analyze to identify beneficial mutations that lead to or improves the target property of the lasso peptides. One or more beneficial mutations are then introduced to the nucleic acid molecule encoding the initial scaffold to produce an improved version of the lasso peptide.

Optionally, in some embodiments, the coding sequence of the improved version of the lasso peptide is further mutated to introduce one or more additional mutations, while maintain the beneficial mutations, in the coding sequence. In some embodiments, a plurality of mutated versions of the coding sequences, each comprising at least one beneficial mutation identified in the first round of screen and at least one additional mutation is provided. These plurality of mutated versions of the coding sequences are then used to produce a second phage display library using, for example, the methods described herein. As such, the second phage display library is enriched with lasso peptides having at least one beneficial mutations. In some embodiments, the second phage display library is subjected to at least one more round of screening to identify improved members having the desirable target property. In some embodiments, additional beneficial mutations can be identified during the second round of the screening, and these additional beneficial mutations can also be used to design improved versions of the lasso peptide.

In some embodiments, additional beneficial mutations are also incorporated into members of a third or further phage display library(ies), which library(ies) can be subjected to a third or further round of screening and selection to identify candidate member(s) having the desirable target property. Additional beneficial mutations can be further identified for the evolution of the initial scaffold toward variants having improved target property. Examples 6 and 7 provide detailed exemplary procedures for directed evolution of lasso peptides.

In some embodiments, a later round of screening is performed at a more stringent condition as compared to an earlier round of screening, such that in the later round of screening, library members exhibiting the target property to a great extent (i.e. a better candidate) can be identified. Various adjustments for obtaining a more stringent screening condition are within the knowledge and skill in the art. For example, in specific embodiments, to identify lasso peptides that specifically binds to a target molecule, a more stringent screening condition can be achieved by performing the screening in the presence of a higher concentration of a molecule known to compete for binding to the target molecule. For example, in specific embodiments, to identify lasso peptides of improved thermal stability, a more stringent screening condition can be achieved by performing the screening at a higher temperature. For example, in specific embodiments, to identify lasso peptides capable of modulating a cellular activity or cell phenotype of interest, a more stringent screening condition can be achieved by performing the screening using less (or at a lower concentration of) candidate lasso peptides. In other embodiments, a more stringent screening condition can be achieved by setting forth a higher threshold for selection (e.g., a lower EC₅₀ or IC₅₀ in an assay measuring modulation of a cellular activity of interest, or a lower CC₅₀ in an assay measuring induced cell death, or a lower K_{d} in a binding assay, etc.).

Furthermore, a number of exemplary methods have been developed for the mutagenesis and diversification of genes and oligonucleotides to introduce into, and/or improve desirable target properties of, specific enzymes, proteins and peptides. Such methods are well known to those skilled in the art. Any of these can be used to alter and/or optimize the activity of a lasso peptide biosynthetic pathway enzyme, protein, or peptide, including a lasso precursor peptide, a lasso core peptide, or a lasso peptide. Such methods include, but are not limited to error-prone polymerase chain reaction (epPCR), which introduces random point mutations by reducing the fidelity of DNA polymerase in PCR reactions (See: Pritchard et al., J. Theor. Biol., 2005, 234:497-509); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res., 2004, 32:e 145; and Fujii et al., Nat. Protoc., 2006, 1, 2493-2497); DNA, Gene, or Family Shuffling, which typically involves digestion of two or more variant genes with nucleases such as DNase I or EndoV to generate a pool of random fragments that are reassembled by cycles of annealing and extension in the presence of DNA polymerase to create a library of chimeric genes (Stemmer, Proc. Natl. Acad. Sci. U.S.A., 1994, 91, 10747-10751; and Stemmer, Nature, 1994, 370, 389-391); Staggered Extension (StEP), which entails template priming followed by repeated cycles of 2-step PCR with denaturation and very short duration of annealing/extension (as short as 5 sec) (Zhao et al., Nat. Biotechnol., 1998, 16, 258-261); Random Priming Recombination (RPR), in which random sequence primers are used to generate many short DNA fragments complementary to different segments of the template (Shao et al., Nucleic Acids Res., 1998, 26, 681-683).

Additional methods include Heteroduplex Recombination, in which linearized plasmid DNA is used to form heteroduplexes that are repaired by mismatch repair (See: Volkov et al, Nucleic Acids Res., 1999, 27:e18; Volkov et al., Methods Enzymol., 2000, 328, 456-463); Random Chimeragenesis on Transient Templates (RACHITT), which employs DNase I fragmentation and size fractionation of single-stranded DNA (ssDNA) (See: Coco et al., Nat. Biotechnol., 2001, 19, 354-359); Recombined Extension on Truncated Templates (RE 1′1), which entails template switching of unidirectionally growing strands from primers in the presence of unidirectional ssDNA fragments used as a pool of templates (See: Lee et al., J. Mol. Cat., 2003, 26, 119-129); Degenerate Oligonucleotide Gene Shuffling (DOGS), in which degenerate primers are used to control recombination between molecules; (Bergquist and Gibbs, Methods Mol. Biol., 2007, 352, 191-204; Bergquist et al., Biomol. Eng., 2005, 22, 63-72; Gibbs et al., Gene, 2001, 271, 13-20); Incremental Truncation for the Creation of Hybrid Enzymes (ITCHY), which creates a combinatorial library with 1 base pair deletions of a gene or gene fragment of interest (See: Ostermeier et al., Proc. Natl. Acad. Sci. U.S.A., 1999, 96, 3562-3567; and Ostermeier et al., Nat. Biotechnol., 1999, 17, 1205-1209); Thio-Incremental Truncation for the Creation of Hybrid Enzymes (THIO-ITCHY), which is similar to ITCHY except that phosphothioate dNTPs are used to generate truncations (See: Lutz et al., Nucleic Acids Res., 2001, 29, E16); SCRATCHY, which combines two methods for recombining genes, ITCHY and DNA Shuffling (See: Lutz et al., Proc. Natl. Acad. Sci. U.S.A., 2001, 98, 11248-11253); Random Drift Mutagenesis (RNDM), in which mutations made via epPCR are followed by screening/selection for those retaining usable activity (See: Bergquist et al., Biomol. Eng., 2005, 22, 63-72); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (See: Wong et al., Biotechnol. J., 2008, 3, 74-82; Wong et al., Nucleic Acids Res., 2004, 32, e26; Wong et al., Anal. Biochem., 2005, 341, 187-189); Synthetic Shuffling, which uses overlapping oligonucleotides designed to encode “all genetic diversity in targets” and allows a very high diversity for the shuffled progeny (See: Ness et al., Nat. Biotechnol., 2002, 20, 1251-1255); Nucleotide Exchange and Excision Technology NexT, which exploits a combination of dUTP incorporation followed by treatment with uracil DNA glycosylase and then piperidine to perform endpoint DNA fragmentation (See: Muller et al., Nucleic Acids Res., 33:e117).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (See: Sieber et al., Nat. Biotechnol., 2001, 19, 456-460); Gene Site Saturation Mutagenesis™ (GSSM™), in which the starting materials include a supercoiled double stranded DNA (dsDNA) plasmid containing an insert and two primers which are degenerate at the desired site of mutations, enabling all amino acid variations to be introduced individually at each position of a protein or peptide (See: Kretz et al., Methods Enzymol., 2004, 388, 3-11); Combinatorial Cassette Mutagenesis (CCM), which involves the use of short oligonucleotide cassettes to replace limited regions with a large number of possible amino acid sequence alterations (See: Reidhaar-Olson et al. Methods Enzymol., 1991, 208, 564-586; Reidhaar-Olson et al. Science, 1988, 241, 53-57); Combinatorial Multiple Cassette Mutagenesis (CMCM), which is essentially similar to CCM and uses epPCR at high mutation rate to identify hot spots and hot regions and then extension by CMCM to cover a defined region of protein sequence space (See: Reetz et al., Angew. Chem. Int. Ed Engl., 2001, 40, 3589-3591); the Mutator Strains technique, in which conditional is mutator plasmids, utilizing the mutD5 gene, which encodes a mutant subunit of DNA polymerase III, to allow a 20 to 4000-fold increase in random and natural mutation frequency during selection and block accumulation of deleterious mutations when selection is not required (See: Selifonova et al., Appl. Environ. Microbiol., 2001, 67, 3645-3649); Low et al., J. Mol. Biol., 1996, 260, 3659-3680).

Additional exemplary methods include Look-Through Mutagenesis (LTM), which is a multidimensional mutagenesis method that assesses and optimizes combinatorial mutations of a selected set of amino acids (See: Rajpal et al., Proc. Natl. Acad. Sci. U.S.A., 2005, 102, 8466-8471); Gene Reassembly, which is a homology-independent DNA shuffling method that can be applied to multiple genes at one time or to create a large library of chimeras (multiple mutations) of a single gene (See: Short, J. M., U.S. Pat. No. 5,965,408, Tunable GeneReassembly™); in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (See: Hayes et al., Proc. Natl. Acad. Sci. U.S.A., 2002, 99, 15926-15931); and Iterative Saturation Mutagenesis (ISM), which involves using knowledge of structure/function to choose a likely site for enzyme improvement, performing saturation mutagenesis at chosen site using a mutagenesis method such as Agilent QuikChange Lightning Site-Directed Mutagenesis (Agilent Technologies; Santa Clara Calif.), screening/selecting for desired properties, and, using improved clone(s), starting over at another site and continue repeating until a desired activity is achieved (See: Reetz et al., Nat. Protoc., 2007, 2, 891-903; Reetz et al., Angew. Chem. Int. Fd Engl., 2006, 45, 7745-7751).

Any of the aforementioned methods for lasso peptide mutagenesis and/or display can be used alone or in any combination to improve the performance of lasso peptide biosynthesis pathway enzymes, proteins, and peptides. Similarly, any of the aforementioned methods for mutagenesis and/or display can be used alone or in any combination to enable the creation of lasso peptide variants which may be selected for improved properties.

In alternative embodiments, the present disclosure provides a method or composition according to any embodiment of the present disclosure, substantially as herein before described, or described herein, with reference to any one of the examples. In alternative embodiments, practicing the present disclosure comprises use of any conventional technique commonly used in molecular biology, microbiology, and recombinant DNA, which are within the skill of the art. Such techniques are known to those of skill in the art and are described in numerous texts and reference works (See e.g., Green and Sambrook, “Molecular Cloning: A Laboratory Manual,” 4th Edition, Cold Spring Harbor, 2012; and Ausubel et al., “Current Protocols in Molecular Biology,” 1987). Unless defined otherwise herein, all technical and scientific terms used herein have the same meaning as commonly understood by one of ordinary skill in the art to which the present disclosure pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, NY (1991) provides those of skill in the art with general dictionaries of many of the terms used in the present disclosure. Although any methods and materials similar or equivalent to those described herein find use in the practice of the present disclosure, the preferred methods and materials are described herein. Accordingly, the terms defined below are more fully described by reference to the Specification as a whole.

### 6. EXAMPLES

TABLE A
The list of protein sequences described in the following Examples 1-9.

| SEQ ID | | | Gen Bank |
|---|---|---|---|
| NO: | Name | A.A. sequence | Accession # |
| 2631 | Fusilassin | WYTAEWGLELIFVFPRFI (W1-E9 cyclized) | N/A |
| | (Thermobifida | | |
| | fusca) | | |
| 2632 | Fusilassin precursor | MEKKKYTAPQLAKVGEFKEATGWYTAEWGLELIF | N/A |
| | A (Thermobifida | VFPRFI | |
| | fusca) | | |
| 2633 | Fusilassin peptidase | MSENVVLQRSNVRLSWRTKWAARCAVGAARLLAR | WP_ |
| | B (Thermobifida | KPPERIRATLLRLRGEVRPATYEEAKAARDAVLAVS | 011291590 |
| | fusca) | LRCAGLRACLQRSLAIALLCRMRGTWATWCVGVPR | |
| | | RPPFIGHAWVEAEGRLVEEGVGYDYFSRLITVD | |
| 2634 | Fusilassin cyclase C | MVGCISPYFAVFPDKDVLGQATDRLPAAQTLASHPS | WP_ |
| | (Thermobifida | GRPWLVGALPADQLLLVEAGERRLAVIGHCSAEPE | 011291592 |
| | fusca) | RLRAELAQIDDVAQFDRIARTLDGSFHLVVVVGDQ | |
| | | MRIQGSVSGLRRVFHAHVGTARIAADRSDVLAAVL | |
| | | GVSPDPDVLALRMFNGLPYPLSELPPWPGVEHVPA | |
| | | WHYLSLGLHDGRHRVVQWWHPPEAELAVTAAAPL | |
| | | LRTALAGAVDTRTRGGGVVSADLSGGLDSTPLCAL | |
| | | AARGPAKVVALTFSSGLDTDDDLRWAKIAHQSFPS | |
| | | VEHVVLSPEDIPGFYAGLDGEFPLLDEPSVAMLSTPR | |
| | | ILSRLHrARAHGSRLHMDGLGGDQLLTGSLSLYHDL | |
| | | LWQRPWTALPLIRGHRLLAGLSLSETFASLADRRDL | |
| | | RAWLADIRHSIATGEPPRRSLFGWDVLPKCGPWLTA | |
| | | EARERVLARFDAVLESLEPLAPTRGRHADLAAIRAA | |
| | | GRDLRLLHQLGSSDLPRMESPFLDDRVVEACLQVR | |
| | | HEGRMNPFEFKSLMKTAMASLLPAEFLTRQSKTDG | |
| | | TPLAAEGFTEQRDRIIQIWRESRLAELGLIHPDVLVER | |
| | | VKQPYSFRGPDWGMELTLTVELWLRSRERVLQGAN | |
| | | GGDNRS | |
| 2635 | Fusilassin RRE | METFGAEFRLRPEISVAQTDYGMVLLDGRSGEYWQ | WP_ |
| | (Thermobifida | LNDTAALIVQRLLDGHSPADVAQFLTSEYEVERTDA | 011291591 |
| | fusca) | ERDIAALVTSLKENGMALP | |
| 2636 | BI-32169 | GLPWGCPSDIPGWNTPWAC (G1-D9 cyclized) | N/A |
| | (Streptomyces sp. | | |
| | DSM 14996) | | |
| 2637 | BI-32169 analog | GLPWGCPNDLFFVNTPFAC (G1-D9 cyclized) | N/A |
| | (Kibdelosporangium | | |
| | sp. MJ126-NF4) | | |
| 2638 | BI-32169 analog | MIKDDEIYEVPTLVEVGDFAELTLGLPWGCPNDLFF | N/A |
| | precursor A | VNTPFAC | |
| | (Kibdelosporangium | | |
| | sp. MJ126-NF4) | | |
| 2639 | Hybrid BI-32169 | MIKDDEIYEVPTLVEVGDFAELTLGLPWGCPSDIPG | N/A |
| | precursor A | WNTPWAC | |
| 2640 | BI-32169 analog | MTMPVAAETTVPLPWHRHITARLATGSARVLIRLRP | WP_ |
| | peptidase B | RRLRVVLRMVSRGARPATAAQALSARQAVVSVSV | 042177890 |
| | (Kibdelosporangium | RCAGQGCLQRAVATALLCRLAGDWPDWCTGFRTR | |
| | sp. MJ126-NF4) | PFRAHAWVEAEGGAVGEPGDMPLFHTVISVRHPAR | |
| | | EAR | |
| 2641 | BI-32169 analog | MRDRRWRAGVRPSTADAGTKGKGLLVGGNEFLVF | WP_ |
| | cyclase C | PDCPVALDAPGGRTVPHASGRPWLVGDWSDDDIVV | 083466052 |
| | (Kibdelosporangium | ISAGTRRLAIVGQARVNVHAVERSLEAAGSVRDLD | |
| | sp. MJ126-NF4) | AVVGTIPGNFHLIASIDGRTRVQGTVSTVRQVFTATI | |
| | | VGTTVAASGPGLLAAATGSRVDGDALALRLVPVVP | |
| | | WPLCLRPVWSGVEQVAAGHWL | |
| 2642 | BI-32169 analog | MTIALTPNVTATDSEDGLVLLNESTGRYWTLNGTG | WP_ |
| | RRE | AATLRLLLAGNSPAQTASRLAERYPDAVDRTQRDV | 042177888 |
| | (Kibdelosporangium | VALLAALRNARLVTSS | |
| | sp. MJ126-NF4) | | |
| 2643 | PelB secretion | MKYLLPTAAAGLLLLAAQPAMAV↓ | N/A |
| | sequence (ssPelB) | | |
| 2644 | TorA secretion | MNNNDLFQASRRRFLAQLGGLTVAGMLGPSLLTPR | N/A |
| | sequence (ssTorA) | RATA↓VAQA | |
| 2645 | TEV cleavage site | ENLYFQ↓G | N/A |
| 2646 | Linker 1 | GAAAKGAAAKGAAAKGAAAK | N/A |
| 2647 | Linker 2 | SGGGGSGGGGSGGGGSGGGGSGGGG | N/A |
| 2648 | Truncated M13 | DCAFHSGFNEDPFVCEYQGQSSDLPQPPVNAGGGSG | NP_510891 |
| | phage p3 (205-406) | GGSGGGSEGGGSEGGGSEGGGSEGGGSGGGSGSGD | |
| | | FDYEKMANANKGAMTENADENALQSDAKGKLDS | |
| | | VATDYGAAIDGFIGDVSGLANGNGATGDFAGSNSQ | |
| | | MAQVGDGDNSPLMNNFRQYLPSLPQSVECRPFVFS | |
| | | AGKPYEFSIDCDKINLFRGVFAFLLYVA | |
| 2649 | M13 phage p8 (24- | AEGDDPAKAAFNSLQASATEYIGYAWAMVVVIVG | NP_510890 |
| | 73) | ATIGIKLFKKFTSKAS | |
| 2650 | Hemolysin A | QGNSLAKNVLSGGKGNDKLYGSEGADLLDGGEGN | WP_ |
| | (HlyA) (806-1024) | DLLKGGYGNDIYRYLSGYGHHIIDDEGGKDDKLSL | 001142370 |
| | | ADIDFRDVAFKREGNDLIMYKAEGNVLSIGHKNGIT | |
| | | FKNWFEKESDDLSNHQIEQIFDKDGRVITPDSLKKAF | |
| | | EYQQSNNKVSYVYGHDASTYGSQDNLNPLINEISKII | |
| | | SAAGNFDVKEERSAASLLQLSGNASDFSYGRNSITL | |
| | | TASA | |
| 2651 | Hemolysin B (HlyB) | MMSKCSSHNSLYALILLAQYHNITVNAETIRHQYNT | WP_ |
| | | HTQDFGVTEWLLAAKSIGLKAKYVEKHFSRLSIISLP | 000987091 |
| | | ALIWRDDGKHYILSRITKDSSRYLVYDPEQHQSLTFS | |
| | | RDEFEKLYQGKVILVTSRATVVGELAKFDFSWFIPS | |
| | | VVKYRRILLEVLTVSAFIQFLALITPLFFQVVMDKVL | |
| | | VHRGFSTLNIITIAFIIVILFEVILTGARTYIFSHTT | |
| | | SRIDVELGAKLFRHLLALPVSYFENRRVGETVARVREL | |
| | | EQIRNFLTGQALTSVLDLFFSVIFFCVMWYYSPQLTLV | |
| | | ILLSLPCYVIWSLFISPLLRRRLDDKFLRNAENQAFLV | |
| | | ETVTAINTIKSMAVSPQMIATWDKQLAGYVASSFRV | |
| | | NLVAMTGQQGIQLIQKSVMVISLWMGAHLVISGEISI | |
| | | GQLIAFNMLAGQVIAPVIRLAHLWQDFQQVGISVER | |
| | | LGDVLNTPVEKKSGRNILPE1QGDIEFKNVRFRYSSD | |
| | | GNVILNNINLYISKGDVIGIVGRSGSGKSTLTKLLQRF | |
| | | YIPETGQILIDGHDLSLADPEWLRRQIGVVLQENILL | |
| | | NRSIIDNITLASPAVSMEQAIEAARLAGAHDFIRELKE | |
| | | GYNTIVGEQGVGLSGGQRQRIAIARALVTNPRILIFD | |
| | | EATSALDYESENIIMKNMSRICKNRTVIIIAHRLSTVK | |
| | | NANRIIVMDNGFISEDGTHKELISKKDSLYAYLYQL | |
| | | QA | |
| 2652 | Hemolysin D | MRFYMKGLWDLVCRYKTVFSDVWKIRHTLDAPVREK | WP_ |
| | (HlyD) | DEYAFLPAHLELIETPVSRRSHFVVWSILLFVIISLL | 100028866 |
| | | LSVLGKVEVVSVANGKFTHSGRSKE1KPIENAIVEKI | |
| | | MVKDGSFVKKNDPLVELTVPGVESDILKSEASLLYE | |
| | | KTEQYRYAILSESIQRNELPEIR1TDFPGGEDNAGGE | |
| | | HFQRVSSLIKEQFMTWQNRKNQKQLTLNKKTVERDA | |
| | | ALARVSLYEHQVSQEGRKLNDFKYLLNKKAVSQHS | |
| | | VMEQENSYIQAKNEHAVWLAQVSQLEKEIELVREEL | |
| | | ALETNIFRSE1IEKHRKSTDNIVLLEHELEKNRQRKA | |
| | | SSFIKAPVSGTVQELNIHTEGGVVTTAETLMIIVPDN | |
| | | DILEVTASVLNKDIGFIQPGQEVVIKVDAYPYTRHGY | |
| | | LTGKVKNITADSVSVPDTGLVFNVIISVDRNDIQGER | |
| | | KKIPVTAGMTVMAE1KTGVRSVISYLLSPLKETINES | |
| | | LRER | |
| 2653 | Enterokinase | DDDDK | N/A |
| | cleavage site (EK) | | |
| 2654 | Truncated maltose- | MKIEEGKLVIWINGDKGYNGLAEVGKKFEKDTGIK | WP_ |
| | binding protein | VTVEHPDKLEEKFPQVAATGDGPDIIFWAHDRFGGY | 052916395 |
| | (MBP) (deletion 2- | AQSGLLAE1TPDKAFQDKLYPFTWDAVRYNGKL1A | |
| | 29) | YPIAVEALSL1YNKDLLPNPPKTWEE1PALDKELKAK | |
| | | GKSALMFNLQEPYFTWPL1AADGGYAFKYENGKYD | |
| | | IKDVGVDNAGAKAGLTFLVDL1KNKHMNADTDYSI | |
| | | AEAAFNKGETAMTINGPWAWSNIDTSKVNYGVTVL | |
| | | PTFKGQPSKPFVGVLSAGINAASPNKELAKEFLENYL | |
| | | LTDEGLEAVNKDKPLGAVALKSYEEELAKDPRIAAT | |
| | | MENAQKGEIMPNIPQMSAFWYAVRTAVINAASGRQ | |
| | | TVDEALKDAQTRITK | |
| 2655 | Capistruin | GTPGFQTPDARVISRFGFN(G1-D9 cyclized) | |
| | (Burkholderia | | |
| | thailandensis) | | |
| 2656 | Capistruin precursor | MVRLLAKLLRSTIHGSNGVSLDAVSSTHGTPGFQTP | WP_ |
| | A | DARVISRFGFN | 009905508 |
| | (Burkholderia | | |
| | thailandensis) | | |
| 2657 | Capistruin peptidase | MTPASHCHIAVFDQAIVALDMQRSRYFLYDEACAK | WP_ |
| | B | AFADHYLDFKPIDAPHALKPLISDRIVVAASPASVPK | 009905509 |
| | (Burkholderia | RIADYRGWAFDAFDSGIWASRTLGERSAAGFEWLP | |
| | thailandensis) | FWRIVRGAVSLKMRGFRALSALDRLARLDAGAEQR | |
| | | ARTDGGPSRTAERYLRASIWSPFRITCLQMSFALATH | |
| | | LRRENVPAQLVIGVRPMPFVAHAWVEIDGRVCGDE | |
| | | PELKKSYGEIYRTPRHDERAGPFGLAA | |
| 2658 | Capistruin cyclase C | MTLLEAGARARAYLRDAHSRIERSLARARTLQEAR | WP_ |
| | (Burkholderia | DTVTRSVWGAYLLVLDEAASGRRLFMPDPLHSVRL | 045600732 |
| | thailandensis) | YYRTDERGRVDVDPRAANLLDRASIDWNLDYLIEF | |
| | | ACTQFGPLDETPFASVRVVPPGCALVVGPDGRCAIE | |
| | | RAWLPRAQAAGDVRASCAAALDDVYSRIAHSHPSV | |
| | | CAALSGGVDSSAGAIFLRKALGANAPLAAVHLYSTS | |
| | | SPDCYERDMAARVADSIGAQLICIDIDRHLPFSERIVR | |
| | | TPPAALNQDMLFLGIDRAVSNALGPSSVLLEGQGGD | |
| | | LLFRAVPDANAVLDALRSNGWSFALRTAEKLAMLH | |
| | | NDSIPRILLMAAKIALRRRLFGQDAPASQQTMSRLFA | |
| | | SSAPRAAAGRSRRHAPRADAPLDESISMLDRFVSIM | |
| | | TPVTDAAYTSRLNPYLAQPVVEAAFGLRSYDSFDHR | |
| | | NDRIVLREIASAHTPVDVLWRRTKGSFGIGFVKGIVS | |
| | | HYDALRELIRDGVLMRSGRLDEAELEHALKAVRVG | |
| | | QNAAAISVALVGCVEVFCASWQNFVTNRHAAVC | |

### 6.1 Example 1: Making M13 Phage Having a Single Lasso Peptide on p3 Coat Protein with Lasso Formation in the Periplasmic Space

This example describes the process for making M13 phage having a single lasso peptide fused to the p3 coat protein, wherein the lasso is formed in the periplasmic space of an E. coli cell.

To display a lasso peptide on the surface of M13 phage, two recombinant DNA plasmids are generated: the ssPelB-fusilassin-TEV-p3 phagemid and the ssTorA-B/ssTorA-C/ssTorA-RRE plasmid as shown in FIG. 3. The phagemid and plasmid vectors are constructed to express the proteins and enzymes for lasso peptide formation and used in conjunction with a helper phage for displaying fusilassin lasso peptide as a p3 fusion protein on M13 phage. Helper phage M13K07 (New England Biolabs, Cat. #N0315S), containing the P15A E. coli replication origin and the kanamycin resistance gene, is used to supply phage structural proteins, such as p2, p3, p5, p6, p7, p8 and p9 for single-stranded phagemid packaging and phage particle maturation. M13K07 caries a gene II mutation that renders it 50-fold less efficient than the recombinant ssPelB-fusilassin-TEV-p3 phagemid vector at producing progeny (+) strands for packaging. Therefore, the vast majority of phage particles contain the ssPelB-fusilassin-TEV-p3 phagemid vector, not the M13KO7 genome.

To generate the ssPelB-fusilassin-TEV-p3 phagemid, the fusilassin precursor sequence A is fused in front of a truncated M13 phage p3 coat protein (residues 205-406) and behind an IPTG-inducible promoter and a PelB secretion sequence (Met-Lys-Tyr-Leu-Leu-Pro-Thr-Ala-Ala-Ala-Gly-Leu-Leu-Leu-Leu-Ala-Ala-Gln-Pro-Ala-Met-Ala↓)(SEQ ID NO: 2643). The TEV protease recognition sequence (Glu-Asn-Leu-Tyr-Phe-Gln↓Gly) (SEQ ID NO: 2645) flanked by two linker sequences, Linker 1 and Linker 2, is then inserted in-flame in between the fusilassin precursor sequence A and the truncated p3 coat protein. The PelB secretion sequence (ssPelB) targets the ssPelB-fusilassin-TEV-p3 fusion protein for periplasmic secretion via the Sec-mediated secretion machinery. And the TEV protease recognition sequence can be cleaved by TEV protease to release fusilassin from the p3 coat protein on the mature M13 phage for validation of lasso conformation by mass spectrometry. The constructed ssPelB-fusilassin-TEV-p3 fusion sequence is then cloned into the pComb3 vector (Creative Biolabs, Cat. #VPT4010), an M13 phagemid containing the pUC E. coli replication origin, the F1 phage replication origin, and the ampicillin resistance gene. Upon the periplasmic secretion of the ssPelB-fusilassin-TEV-p3 fusion protein, the PelB secretion sequence is cleaved off and the fusilassin precursor peptide A fused to the p3 coat protein is subsequently inserted into the inner membranes of E. coli.

To generate the ssTorA-B/ssTorA-C/ssTorA-RRE plasmid, the fusilassin peptidase (B), cyclase (C) and RiPP Recognition Element (RRE) are individually cloned behind an IPTG-inducible promoter and a TorA secretion sequence (ssTorA) on a separate plasmid containing the chloramphenicol resistance gene to create three ssTorA fusion proteins, ssTorA-B, ssTorA-C and ssTorA-RRE. The TorA secretion sequence targets the folded fusilassin processing enzymes B, C and RRE to the periplasm via the Tat secretion machinery. Upon the periplasmic secretion, the TorA secretion sequence is cleaved off to yield untagged B, C and RRE proteins that can catalyze lasso peptide formation in the periplasm.

To produce the M13 phage displaying lasso peptide, the fusilassin phagemid and the ssTorA-B/ssTorA-C/ssTorA-RRE plasmid are first transformed into E. coli SS320 (Lucigen, Cat #60512-1) via electroporation following the manufacturer's instructions. The E. coli SS320 strain contains the tetracycline resistance gene as a selection marker. Following transformation, the E. coli cells are recovered in 1 mL of 2×YT medium for 1 hour at 37° C. in an incubator shaker at 250 rpm. After one-hour incubation, one-tenth of the culture (100 μL) is spread on 2×YT agar containing 100 μg/mL ampicillin, 25 μg/mL chloramphenicol, and 10 μg/mL tetracycline. The 2×YT agar plate is incubated overnight at 37° C. to yield single colonies. The next day, a single isolated colony from the overnight plate is used to prepare a 5 mL overnight culture in 2×YT containing 2% (w/v) glucose, 100 μg/mL ampicillin, 25 μg/mL chloramphenicol, and 10 μg/mL tetracycline. This overnight culture is subsequently used to inoculate a fresh culture of 2×YT at 1% v/v (1 mL/100 mL) containing 2% (w/v) glucose and the same antibiotics. The freshly inoculated culture is grown at 37° C. in an incubator shaker at 250 rpm for 4 to 5 hours with OD₆₀₀ monitored every 30 minutes. When the culture reaches mid-log phase (OD₆₀₀=0.4-0.5), helper phage M13KO7 stock at 10¹² pfu/mL is added to the culture at a ratio of 1:500 (v/v) helper phage:culture media. After addition of helper phage, the culture is further incubated at 37° C. in an incubator shaker at 250 rpm for 1 hour to allow phage transfection. Following the one-hour incubation, kanamycin is added at 60 μg/mL to remove any uninfected E. coli cells. To initiate phage production, the expression of ssPelB-fusilassin-TEV-p3, ssTorA-B, ssTorA-C and ssTorA-RRE is induced with IPTG at 1 mM. The induced culture is then incubated at 28° C. in an incubator shaker at 250 rpm for 24 hours to produce phage. During the phage assembly, the simultaneous presence of two to three copies of the wild-type p3 coat protein (encoded by the helper phage) facilitates efficient assembly of infective phage. As the result, the fusilassin-TEV-p3 fusion protein is displayed at two to three copies per phage particle.

Following the production of phage, the E. coli cells are removed by two successive centrifugation steps (14,000×g, 15 minutes, 4° C.). The upper 80% of the supernatant is collected and mixed with one-fourth volume of polyethylene glycol 8000 (PEG 8000)/NaCl solution (20% PEG 8000, 2.5 M NaCl). The thoroughly mixed sample is placed on ice overnight to precipitate the phage. After overnight incubation on ice, the phage is pelleted by centrifugation at 11,000×g for 10 minutes at 4° C. The supernatant is discarded, and the pellet is resuspended in 2 mL of PBS buffer (pH=7.4). The resuspended sample is then centrifuged again at 14,000×g for 15 minutes at 4° C. to pellet insoluble debris. After precipitation of insoluble debris, the supernatant is transferred to a fresh tube and the phage is precipitated for the second time by adding one-fourth volume of polyethylene glycol 8000 (PEG 8000)/NaCl solution (20% PEG 8000, 2.5 M NaCl). The sample is then thoroughly mixed and placed on ice for at least two hours. The phage is again pelleted by centrifugation at 11,000×g for 10 minutes at 4° C. The supernatant is discarded, and the pelleted phage is resuspended in 500 mL of PBS buffer (pH=7.4). The concentration of the phage is determined by UV absorbance as described by Day and Wiseman (The Single-Stranded DNA Phages, Cold Spring Harbor, N.Y., 1978, p 605): phage concentration (phages/mL)=((A₂₆₉−A320)×6×10¹⁶)/(phage genome size in nt)×dilution factor. The resuspended phage supernatant is passed through a 0.22 μm filter for sterilization.

To detect display of fusilassin lasso peptide on the mature phage, the filtered M13 phage is treated with TEV protease (Sigma Cat. #T4455) to release fusilassin lasso peptide following the manufacturer's instructions. The protease digestion reaction is then treated with an equal volume of methanol, thoroughly mixed and centrifuged to precipitate insoluble debris. The soluble faction which contains released fusilassin lasso peptide fused to Linker 1 and part of TEV protease recognition site (Fusilassin-Linker 1-Glu-Asn-Leu-Tyr-Phe-Gln) is concentrated and subjected to MALDT-TOF MS analysis. The presence of the ssPelB-fusilassin-TEV-p3 DNA sequence in the mature phage is also independently detected by PCR amplification and DNA sequencing.

### 6.2 Example 2: Making M13 Phage Having a Single Lasso Peptide on p8 Coat Protein with Lasso Formation in the Periplasmic Space

This example describes methods for making M13 phage having a single lasso peptide on p8 coat protein, wherein the lasso is formed in the periplasmic space of an E. coli cell.

To display a lasso peptide on the surface of M13 phage, two recombinant DNA plasmids are generated: the ssPelB-fusilassin-TEV-p8 phagemid and the ssTorA-B/ssTorA-C/ssTorA-RRE plasmid. The phagemid and plasmid vectors are constructed to express the proteins and enzymes for lasso peptide formation and used in conjunction with a helper phage for displaying fusilassin lasso peptide as a p8 fusion protein on M13 phage. Helper phage M13KO7 (New England Biolabs, Cat. #N0315S), containing the P15A E. coli replication origin and the kanamycin resistance gene, is used to supply the phage structural proteins, such as p2, p3, p5, p6, p7, p8 and p9 for single-stranded phagemid packaging and phage particle maturation. M13K07 carries a gene II mutation that renders it 50-fold less efficient than the recombinant fusilassin-p8 phagemid vector at producing progeny (+) strands for packaging. Therefore, the vast majority of phage particles contain the ssPelB-fusilassin-TEV-p8 phagemid vector, not the M13KO7 genome.

To generate the ssPelB-fusilassin-TEV-p8 phagemid, the fusilassin precursor sequence A is fused to the N terminus of an M13 phage p8 coat protein (residues 24-73) and behind an IPTG-inducible promoter and a PelB secretion sequence (Met-Lys-Tyr-Leu-Leu-Pro-Thr-Ala-Ala-Ala-Gly-Leu-Leu-Leu-Leu-Ala-Ala-Gln-Pro-Ala-Met-Ala↓)(SEQ ID NO: 2643). The TEV protease recognition sequence (Glu-Asn-Leu-Tyr-Phe-Gln↓Gly) (SEQ ID NO: 2645) flanked by two linker sequences, Linker 1 and Linker 2, is then inserted in-flame in between the fusilassin precursor sequence A and the p8 coat protein. The PelB secretion sequence (ssPelB) targets the ssPelB-fusilassin-TEV-p8 fusion protein for periplasmic secretion via the Sec-mediated secretion machinery. And the TEV protease recognition sequence can be cleaved by TEV protease to release fusilassin from the p8 coat protein on the mature M13 phage for validation of lasso conformation by mass spectrometry. The constructed ssPelB-fusilassin-TEV-p8 fusion sequence is then cloned into the pComb8 vector (Creative Biolabs, Cat. #VPT4010), an M13 phagemid containing the pUC E. coli replication origin, the F1 phage replication origin, and the ampicillin resistance gene. Upon the periplasmic secretion of the ssPelB-fusilassin-TEV-p8 fusion protein, the PelB secretion sequence is cleaved off and the fusilassin precursor peptide A fused to the p8 coat protein is subsequently inserted into the inner membranes of E. coli.

To generate the ssTorA-B/ssTorA-C/ssTorA-RRE plasmid, the fusilassin peptidase (B), cyclase (C) and RiPP Recognition Element (RRE) are individually cloned behind an IPTG-inducible promoter and a TorA secretion sequence (ssTorA) on a separate plasmid containing the chloramphenicol resistance gene to create three ssTorA fusion proteins, ssTorA-B, ssTorA-C and ssTorA-RRE. The TorA secretion sequence targets the folded fusilassin processing enzymes B, C and RRE to the periplasm via the Tat secretion machinery. Upon the periplasmic secretion, the TorA secretion sequence is cleaved off to yield untagged B, C and RRE proteins that can catalyze lasso peptide formation in the periplasm.

To produce the M13 phage displaying lasso peptide, the fusilassin phagemid and the ssTorA-B/ssTorA-C/ssTorA-RRE plasmid are first transformed into E. coli SS320 (Lucigen, Cat #60512-1) via electroporaiion following the manufacturer's instructions. The E. coli SS320 strain contains the tetracycline resistance gene as a selection marker. Following transformation, the E. coli cells are recovered in 1 mL of 2×YT medium for 1 hour at 37° C. in an incubator shaker at 250 rpm. After one-hour incubation, one-tenth of the culture (100 μL) is spread on 2×YT agar containing 100 μg/mL ampicillin, 25 μg/mL chloramphenicol, and 10 μg/mL tetracycline. The 2×YT agar plate is incubated overnight at 37° C. to yield single colonies. The next day, a single isolated colony from the overnight plate is used to prepare a 5 mL overnight culture in 2×YT containing 2% (w/v) glucose, 100 μg/mL ampicillin, 25 μg/mL chloramphenicol, and 10 μg/mL tetracycline. This overnight culture is subsequently used to inoculate a fresh culture of 2×YT at 1% v/v (1 mL/100 mL) containing 2% (w/v) glucose and the same antibiotics. The freshly inoculated culture is grown at 37° C. in an incubator shaker at 250 rpm for 4 to 5 hours with OD₆₀₀ monitored every 30 minutes. When the culture reaches mid-log phase (OD600=0.4-0.5), helper phage M13KO7 stock at 10¹² pfu/mL is added to the culture at a ratio of 1:500 (v/v) helper phage:culture media. After addition of helper phage, the culture is further incubated at 37° C. in an incubator shaker at 250 rpm for 1 hour to allow phage transfection. Following the one-hour incubation, kanamycin is added at 60 μg/mL to remove any uninfected E. coli cells. To initiate phage production, the expression of ssPelB-fusilassin-p8, ssTorA-B, ssTorA-C and ssTorA-RRE is induced with IPTG at 1 mM. The induced culture is then incubated at 28° C. in an incubator shaker at 250 rpm for 24 hours to produce phage. During the phage assembly, the simultaneous presence of the wild-type p8 coat protein (encoded by the helper phage) facilitates efficient assembly of infective phage. As the result, the fusilassin-TEV-p8 fusion protein is displayed at approximately two hundred copies per phage particle. Following the production of phage, the E. coli cells are removed by two successive centrifugation steps (14,000×g, 15 minutes, 4° C.). The upper 80% of the supernatant is collected and mixed with one-fourth volume of polyethylene glycol 8000 (PEG 8000)/NaCl solution (20% PEG 8000, 2.5 M NaCl). The thoroughly mixed sample is placed on ice overnight to precipitate the phage. After overnight incubation on ice, the phage is pelleted by centrifugation at 11,000×g for 10 minutes at 4° C. The supernatant is discarded, and the pellet is resuspended in 2 mL of PBS buffer (pH=7.4). The resuspended sample is then centrifuged again at 14,000×g for 15 minutes at 4° C. to pellet insoluble debris. After precipitation of insoluble debris, the supernatant is transferred to a fresh tube and the phage is precipitated for the second time by adding one-fourth volume of polyethylene glycol 8000 (PEG 8000)/NaCl solution (20% PEG 8000, 2.5 M NaCl). The sample is then thoroughly mixed and placed on ice for at least two hours. The phage is again pelleted by centrifugation at 11,000×g for 10 minutes at 4° C. The supernatant is discarded, and the pelleted phage is resuspended in 500 mL of PBS buffer (pH=7.4). The concentration of the phage is determined by UV absorbance as described by Day and Wiseman (The Single-Stranded DNA Phages, Cold Spring Harbor, N.Y., 1978, p 605): phage concentration (phages/mL)=((A₂₆₉−A₃₂₀)×6×10¹⁶)/(phage genome size in nt)×dilution factor. The resuspended phage supernatant is passed through a 0.22 μm filter for sterilization.

To detect display of fusilassin lasso peptide on the mature phage, the filtered M13 phage is treated with TEV protease (Sigma Cat. #T4455) to release fusilassin lasso peptide following the manufacturer's instructions. The protease digestion reaction is then treated with an equal volume of methanol, thoroughly mixed and centrifuged to precipitate insoluble debris. The soluble faction which contains released fusilassin lasso peptide fused to Linker 1 and part of TEV protease recognition site (Fusilassin-Linker 1-Glu-Asn-Leu-Tyr-Phe-Gln) is concentrated and subjected to MALDT-TOF MS analysis. The presence of the PelB-fusilassin-TEV-p8 DNA sequence in the mature phage is also independently detected by PCR amplification and DNA sequencing.

### 6.3 Example 3: Making M13 Phage Having a Single Lasso Peptide on p3 Coat Protein with Lasso Formation in the Extracellular Space

This example describes methods for making M13 phage having a single lasso peptide on p3 coat protein, wherein the lasso is formed in the extracellular space of an E. coli cell.

To display a lasso peptide on the surface of M13 phage, generate two recombinant DNA plasmids are generated: the ssPelB-fusilassin-TEV-p3 phagemid and the B-HlyA/C-HlyA/RRE-HlyA plasmid as shown in FIG. 4. The phagemid and plasmid vectors are constructed to express the proteins and enzymes for lasso peptide formation and used in conjunction with a helper phage for displaying fusilassin lasso peptide as a p3 fusion protein on M13 phage. Helper phage M13KO7 (New England Biolabs, Cat. #N0315 S), containing the PISA E. coli replication origin and the kanamycin resistance gene, is used to supply the phage structural proteins, such as p2, p3, p5, p6, p7, p8 and p9 for single-stranded phagemid packaging and phage particle maturation. M13KO7 carves a gene II mutation that renders it 50-fold less efficient than the recombinant ssPelB-fusilassin-TEV-p3 phagemid vector at producing progeny (+) strands for packaging. Therefore, the vast majority of phage particles contain the ssPelB-fusilassin-TEV-p3 phagemid vector, not the M13KO7 genome.

To generate the ssPelB-fusilassin-TEV-p3 phagemid, the fusilassin precursor sequence A is fused to the N terminus of a truncated M13 phage p3 coat protein (residues 205-406) and behind an IPTG-inducible promoter and a PelB secretion sequence (Met-Lys-Tyr-Leu-Leu-Pro-Thr-Ala-Ala-Ala-Gly-Leu-Leu-Leu-Leu-Ala-Ala-Gln-Pro-Ala-Met-Ala.↓)(SEQ ID NO: 2643). The TEV protease recognition sequence (Glu-Asn-Leu-Tyr-Phe-Gln↓Gly) (SEQ ID NO: 2645) flanked by two linker sequences, Linker 1 and Linker 2, is then inserted in-frame in between the fusilassin precursor sequence A and the truncated p3 coat protein. The PelB secretion sequence (ssPelB) targets the ssPelB-fusilassin-TEV-p3 fusion protein for periplasmic secretion via the Sec-mediated secretion machinery. And the TEV protease recognition sequence can be cleaved by TEV protease to release fusilassin from the p3 coat protein on the mature M13 phage for validation of lasso conformation by mass spectrometry. The constructed ssPelB-fusilassin-TEV-p3 fusion sequence is then cloned into the pComb3 vector (Creative Biolabs, Cat. #VPT4010), an M13 phagemid containing the pUC E. coli replication origin, the F1 phage replication origin, and the ampicillin resistance gene. Upon the periplasmic secretion of the ssPelB-fusilassin-TEV-p3 fusion protein, the PelB secretion sequence is cleaved off and the fusilassin precursor peptide A fused to the p3 coat protein is subsequently inserted into the inner membranes of E. coli and incorporated into the phage particle during phage assembly.

To generate the B-HyA/C-HlyA/RRE-HlyA plasmid, the fusilassin peptidase (B), cyclase (C) and RiPP Recognition Element (RRE) are fused in-frame with an enterokinase cleavage site (EK)(Asp-Asp-Asp-Asp-Lys) (SEQ ID NO:2653) and the C-terminal portion of HlyA (residues 806-1024) to create three fusion sequences, B-EK-HlyA, C-EK-HlyA and RRE-EK-HlyA, each of which is independently expressed by an IPTG-inducible promoter. The most C temrinal portion of HlyA sequence (residues 965-1024) is a secretion signal that directs the extracellular secretion of the three fusion proteins via the alpha-hemolysin secretion complex, composed of HlyB, HlyD and TolC, spanning across both the inner and outer membranes. TolC is an endogenous E. coli outer membrane protein. To supply HlyB and HlyD, a HlyB/HlyD gene expression cassette is cloned into the same plasmid under a constitutive promoter. Upon the extracellular secretion, the fused HlyA sequence can be cleaved off by the addition of recombinant enterokinase (EMD Millipore, Cat. #69066-3) to yield untagged B, C and RRE proteins, which can process the fusilassin precursor peptide A fused to p3 coat protein and catalyze lasso peptide formation on the mature phage in the extracellular space.

To produce the M13 phage displaying lasso peptide, the fusilassin phagemid and the B-EK-HlyA/C-EK-HlyA/RRE-EK-HlyA plasmid are first transformed into E. coli SS320 (Lucigen, Cat #60512-1) via electroporation following the manufacturer's instructions. The E. coli SS320 strain contains the tetracycline resistance gene as a selection marker. Following transformation, the E. coli cells are recovered in 1 mL of 2×YT medium for 1 hour at 37° C. in an incubator shaker at 250 rpm. After one-hour incubation, one tenth of the culture (100 μL) is spread on 2×YT agar containing 100 μg/mL ampicillin, 25 μg/mL chloramphenicol, and 10 μg/mL tetracycline. The 2×YT agar plate is incubated overnight at 37° C. to yield single colonies. The next day, a single isolated colony from the overnight plate is used to prepare a 5 mL overnight culture in 2×YT containing 2% (w/v) glucose, 100 μg/mL ampicillin, 25 μg/mL chloramphenicol, and 10 μg/mL tetracycline. This overnight culture is subsequently used to inoculate a fresh culture of 2×YT at 1% v/v (1 mL/100 mL) containing 2% (w/v) glucose and the same antibiotics. The freshly inoculated culture is grown at 37° C. in an incubator shaker at 250 rpm for 4 to 5 hours with OD₆₀₀ monitored every 30 minutes. When the culture reaches mid-log phase (OD₆₀₀=0.4-0.5), helper phage M13K07 stock at 10¹² pfu/mL is added to the culture at a ratio of 1:500 (v/v) helper phage:culture media. After addition of helper phage, the culture is further incubated at 37° C. in an incubator shaker at 250 rpm for 1 hour to allow phage transfection. Following the one-hour incubation, kanamycin is added at 60 μg/mL to remove any uninfected E. coli cells. To initiate phage production, the expression of ssPelB-fusilassin-TEV-p3, B-EK-HlyA, C-EK-HlyA and RRE-EK-HlyA is induced with IPTG at 1 mM. The induced culture is then incubated at 28° C. in an incubator shaker at 250 rpm for 24 hours to produce phage. During the phage assembly, the simultaneous presence of two to three copies of the wild-type p3 coat protein (encoded by the helper phage) facilitates efficient assembly of infective phage. As the result, the fusilassin precursor peptide A-TEV-p3 fusion protein is displayed at two to three copies per phage particle. To catalyze the formation of fusilassin lasso peptide on the mature phage, recombinant enterokinase (EMD Millipore, Cat. #69066-3) is added to the culture media to cleave off the fused HlyA sequence. These extracellular B, C and RRE proteins can then catalyze lasso peptide formation on the mature phage.

Following the production of phage, the E. coli cells are removed by two successive centrifugation steps (14,000×g, 15 min, 4° C.). The upper 80% of the supernatant is collected and mixed with one-fourth volume of polyethylene glycol 8000 (PEG 8000)/NaCl solution (20% PEG 8000, 2.5 M NaCl). The thoroughly mixed sample is placed on ice overnight to precipitate the phage. After overnight incubation on ice, the phage is pelleted by centrifugation at 11,000×g for 10 minutes at 4° C. The supernatant is discarded, and the pellet is resuspended in 2 mL of PBS buffer (pH=7.4). The resuspended sample is then centrifuged again at 14,000×g for 15 minutes at 4° C. to pellet insoluble debris. After precipitation of insoluble debris, the supernatant is transferred to a fresh tube and the phage is precipitated for the second time by adding one-fourth volume of polyethylene glycol 8000 (PEG 8000)/NaCl solution (20% PEG 8000, 2.5 M NaCl). The sample is then thoroughly mixed and placed on ice for at least two hours. The phage is again pelleted by centrifugation at 11,000×g for 10 minutes at 4° C. The supernatant is discarded, and the pelleted phage is resuspended in 500 mL of PBS buffer (pH=7.4). The concentration of the phage is determined by UV absorbance as described by Day and Wiseman (The Single-Stranded DNA Phages, Cold Spring Harbor, N.Y., 1978, p 605): phage concentration (phages/mL)=((A₂₆₉−A₃₂₀)×6×10¹⁶)/(phage genome size in nt)×dilution factor. The resuspended phage supernatant is passed through a 0.22 μm filter for sterilization.

To detect display of fusilassin lasso peptide on the mature phage, the filtered M13 phage is treated with TEV protease (Sigma Cat. #T4455) to release fusilassin lasso peptide following the manufacturer's instructions. The protease digestion reaction is then treated with an equal volume of methanol, thoroughly mixed and centrifuged to precipitate insoluble debris. The soluble faction which contains released fusilassin lasso peptide fused to Linker 1 and part of TEV protease recognition site (Fusilassin-Linker 1-Glu-Asn-Leu-Tyr-Phe-Gln) is concentrated and subjected to MALDT-TOF MS analysis. The presence of the ssPelB-fusilassin-TEV-p3 DNA sequence in the mature phage is also independently detected by PCR amplification and DNA sequencing.

### 6.4 Example 4: Making M13 Phage Having a Single Lasso Peptide on p3 Coat Protein with Lasso Formation Catalyzed by Purified Peptidase (B), Cyclase (C) and RRE

This example describes methods for making M13 phage having a single lasso peptide on p3 coat protein, wherein the lasso formation is catalyzed by purified peptidase (B), cyclase (C) and RRE.

To display a lasso peptide on the surface of M13 phage, two recombinant DNA plasmids are generated: the ssPelB-fusilassin-TEV-p3 phagemid shown in FIG. 4 and the MBP-B/MBP-C/MBP-RRE plasmid as shown in FIG. 5. The phagemid and plasmid vectors are constructed to express the proteins and enzymes for lasso peptide formation and used in conjunction with a helper phage for displaying fusilassin lasso peptide as a p3 fusion protein on M13 phage. Helper phage M13K07 (New England Biolabs, Cat. #N0315 S), containing the P 15A E. coli replication origin and the kanamycin resistance gene, is used to supply the phage structural proteins, such as p2, p3, p5, p6, p7, p8 and p9 for single-stranded phagemid packaging and phage particle maturation. M13KO7 carries a gene II mutation that renders it 50-fold less efficient than the recombinant ssPelB-fusilassin-TEV-p3 phagemid vector at producing progeny (+) strands for packaging. Therefore, the vast majority of phage particles contain the ssPelB-fusilassin-TEV-p3 phagemid vector, not the M13KO7 genome.

To generate the ssPelB-fusilassin-TEV-p3 phagemid, the fusilassin precursor sequence A is fused to the N terminus of a truncated M13 phage p3 coat protein (residues 205-406) and behind an IPTG-inducible promoter and a PelB secretion sequence (Met-Lys-Tyr-Leu-Leu-Pro-Thr-Ala-Ala-Ala-Gly-Leu-Leu-Leu-Leu-Ala-Ala-Gln-Pro-Ala-Met-Ala↓)(SEQ ID NO:2643). The TEV protease recognition sequence (Glu-Asn-Leu-Tyr-Phe-Gln↓Gly) (SEQ ID NO:2645) flanked by two linker sequences, Linker 1 and Linker 2, is then inserted in-frame in between the fusilassin precursor sequence A and the truncated p3 coat protein. The PelB secretion sequence (ssPelB) targets the ssPelB-fusilassin-TEV-p3 fusion protein for periplasmic secretion via the Sec-mediated secretion machinery. And the TEV protease recognition sequence can be cleaved by TEV protease to release fusilassin from the p3 coat protein on the mature M13 phage for validation of lasso conformation by mass spectrometry. The constructed ssPelB-fusilassin-TEV-p3 fusion sequence is then cloned into the pComb3 vector (Creative Biolabs, Cat. #VPT4010), an M13 phagemid containing the pUC E. coli replication origin, the F1 phage replication origin, and the ampicillin resistance gene. Upon the periplasmic secretion of the ssPelB-fusilassin-TEV-p3 fusion protein, the PelB secretion sequence is cleaved off and the fusilassin precursor peptide A fused to the p3 coat protein is subsequently inserted into the inner membranes of E. coli and incorporated into the phage particle during phage assembly.

To generate the recombinant peptidase (B), cyclase (C) and RRE, the truncated maltose binding protein (MBP) devoid of the secretion sequence residues 2-29 is individually fused in-flame with B, C and RRE to created three fusion sequences, MBP-B, MBP-C and MBP-RRE. Each of the three fusion sequences is cloned behind an IPTG-inducible promoter of an E. coli expression vector containing the chloramphenicol resistance gene. To express the fusion proteins, the three expression vectors are individually transformed into E. coli BL21 and induced with 1 mM IPTG for 16 hours at 29° C. The recombinant MBP-B, MBP-C and MBP-RRE proteins are purified using pMAL™ Protein Fusion and Purification System (New England Biolabs, Cat. #E8200S) following the manufacturer's instructions.

To produce the M13 phage displaying lasso peptide, the ssPelB-fusilassin-TEV-p3 phagemid is first transformed into E. coli SS320 (Lucigen, Cat #60512-1) via electroporaiion following the manufacturer's instructions. The E. coli SS320 strain contains the tetracycline resistance gene as a selection marker. Following transformation, the E. coli cells are recovered in 1 mL of 2×YT medium for 1 hour at 37° C. in an incubator shaker at 250 rpm. After one-hour incubation, one-tenth of the culture (100 μL) is spread on 2×YT agar containing 100 μg/mL ampicillin and 10 μg/mL tetracycline. The 2×YT agar plate is incubated overnight at 37° C. to yield single colonies. The next day, a single isolated colony from the overnight plate is used to prepare a 5 mL overnight culture in 2×YT containing 2% (w/v) glucose, 100 μg/mL ampicillin and 10 μg/mL tetracycline. This overnight culture is subsequently used to inoculate a fresh culture of 2×YT at 1% v/v (1 mL/100 mL) containing 2% (w/v) glucose and the same antibiotics. The freshly inoculated culture is grown at 37° C. in an incubator shaker at 250 rpm for 4 to 5 hours with OD₆₀₀ monitored every 30 minutes. When the culture reaches mid-log phase (OD₆₀₀=0.4-0.5), helperphage M13K07 stock at 10¹² pfu/mL is added to the culture at a ratio of 1:500 (v/v) helper phage:culture media. After addition of helper phage, the culture is further incubated at 37° C. in an incubator shaker at 250 rpm for 1 hour to allow phage transfection. Following the one-hour incubation, kanamycin is added at 60 μg/mL to remove any uninfected E. coli cells. To initiate phage production, the expression of ssPelB-fusilassin-TEV-p3 is induced with IPTG at 1 mM. The induced culture is then incubated at 28° C. in an incubator shaker at 250 rpm for 24 hours to produce phage. During the phage assembly, the simultaneous presence of two to three copies of the wild-type p3 coat protein (encoded by the helper phage) facilitates efficient assembly of infective phage. As the result, the fusilassin precursor peptide A-TEV-p3 fusion protein is displayed at two to three copies per phage particle.

Following the production of phage, the E. coli cells are removed by two successive centrifugation steps (14,000×g, 15 min, 4° C.). The upper 80% of the supernatant is collected and mixed with one-fourth volume of polyethylene glycol 8000 (PEG 8000)/NaCl solution (20% PEG 8000, 2.5 M NaCl). The thoroughly mixed sample is placed on ice overnight to precipitate the phage. After overnight incubation on ice, the phage is pelleted by centrifugation at 11,000×g for 10 minutes at 4° C. The supernatant is discarded, and the pellet is resuspended in 2 mL of PBS buffer (pH=7.4). The resuspended sample is then centrifuged again at 14,000×g for 15 minutes at 4° C. to pellet insoluble debris. After precipitation of insoluble debris, the supernatant is transferred to a fresh tube and the phage is precipitated for the second time by adding one-fourth volume of polyethylene glycol 8000 (PEG 8000)/NaCl solution (20% PEG 8000, 2.5 M NaCl). The sample is then thoroughly mixed and placed on ice for at least two hours. The phage is again pelleted by centrifugation at 11,000×g for 10 minutes at 4° C. The supernatant is discarded, and the pelleted phage is resuspended in 500 mL of PBS buffer (pH=7.4). The concentration of the phage is determined by UV absorbance as described by Day and Wiseman (The Single-Stranded DNA Phages, Cold Spring Harbor, N.Y., 1978, p 605): phage concentration (phages/mL)=((A₂₆₉−A₃₂₀)×6×10¹⁶)/(phage genome size in nt)×dilution factor. The resuspended phage supernatant is passed through a 0.22 μm filter for sterilization.

To catalyze the formation of fusilassin lasso peptide on the mature phage, recombinant MBP-B, MBP-C and MBP-RRE proteins are added to the sterilized phage sample in a buffer containing 50 mM Tris-HCl pH 7.5, 125 mM NaCl, 20 mM MgCl₂, 10 mM DTT, and 5 mM ATP. The sample is incubated at 29° C. for 16 hours to catalyze the formation of fusilassin lasso peptide. Following the 16-hour incubation, the sample is passing through an amylose resin column (New England Biolabs, Cat. #E8021 S) to remove the recombinant MBP-B, MBP-C and MBP-RRE proteins. The sample containing the mature phage displaying fusilassin lasso peptide is subject to another around of precipitation and sterilization as described in the previous paragraph.

To detect display of fusilassin lasso peptide on the mature phage, the filtered M13 phage is treated with TEV protease (Sigma Cat. #T4455) to release fusilassin lasso peptide following the manufacturer's instructions. The protease digestion reaction is then treated with an equal volume of methanol, thoroughly mixed and centrifuged to precipitate insoluble debris. The soluble faction which contains released fusilassin lasso peptide fused to Linker 1 and part of TEV protease recognition site (Fusilassin-Linker 1-Glu-Asn-Leu-Tyr-Phe-Gln) is concentrated and subjected to MALDT-TOF MS analysis. The presence of the ssPelB-fusilassin-TEV-p3 DNA sequence in the mature phage is also independently detected by PCR amplification and DNA sequencing.

### 6.5 Example 5: Making M13 Phage Display Library Having Lasso Peptides on p3 Coat Protein with Lasso Formation in the Periplasmic Space

This example describes methods for making M13 phage display library having lasso peptides on p3 coat protein, wherein the lasso is formed in the periplasmic space of an E. coli cell.

To produce an M13 phage library displaying wild-type and mutant fusilassin lasso peptides, a ssPelB-fusilassin A*-TEV-p3 phagemid library is generated and the ssTorA-B/ssTorA-C/ssTorA-RRE plasmid as shown in FIG. 3. The phagemid library and plasmid vectors are constructed to express the proteins and enzymes for lasso peptide formation and used in conjunction with a helper phage for displaying both wild-type and mutant fusilassin lasso peptides as a p3 fusion protein on M13 phage. Helper phage M13KO7 (New England Biolabs, Cat. #N0315S), containing the P15A E. coli replication origin and the kanamycin resistance gene, is used to supply the phage structural proteins, such as p2, p3, p5, p6, p7, p8 and p9 for single-stranded phagemid packaging and phage particle maturation. M13K07 carries a gene II mutation that renders it 50-fold less efficient than the recombinant ssPelB-fusilassin A*-TEV-p3 phagemid vector at producing progeny (+) strands for packaging. Therefore, the vast majority of phage particles contain the PelB-fusilassin A*-TEV-p3 phagemid vector, not the M13KO7 genome.

To generate the ssPelB-fusilassin A*-TEV-p3 phagemid library, the DNA sequences encoding either wild-type or mutant fusilassin precursor peptides (fusilassin A*) are individually synthesized and arrayed on 96-well plates by Twist Bioscience, Corp. The synthesized DNA sequences are cloned into a modified phagemid derived from pComb3 vector (Creative Biolabs, Cat. #VPT4010), an M13 phagemid containing the pUC E. coli replication origin, the F1 phage replication origin, and the ampicillin resistance gene. The resulting phagemid library expresses wild-type or mutant fusilassin precursor peptides as a PelB-fusilassin A*-TEV-p3 fusion protein from an IPTG-inducible promoter. The PelB secretion sequence (ssPelB) targets the ssPelB-fusilassin A*-TEV-p3 fusion protein for periplasmic secretion via the Sec-mediated secretion machinery. And the TEV protease recognition sequence, flanked by two linker sequences, Linker 1 and Linker 2, can be cleaved by TEV protease to release lasso peptides from the p3 coat protein on the mature M13 phage for validation of lasso conformation by mass spectrometry. Upon the periplasmic secretion of the ssPelB-fusilassin A*-TEV-p3 fusion protein, the PelB secretion sequence is cleaved off and each fusilassin precursor A* peptide fused to the p3 coat protein is subsequently inserted into the inner membranes of E. coli.

To generate the ssTorA-B/ssTorA-C/ssTorA-RRE plasmid, the fusilassin peptidase (B), cyclase (C) and RiPP Recognition Element (RRE) are individually cloned behind an IPTG-inducible promoter and a TorA secretion sequence (ssTorA) on a separate plasmid containing the chloramphenicol resistance gene to create three ssTorA fusion proteins, ssTorA-B, ssTorA-C and ssTorA-RRE. The TorA secretion sequence targets the folded fusilassin processing enzymes B, C and RRE to the periplasm via the Tat secretion machinery. Upon the periplasmic secretion, the TorA secretion sequence is cleaved off to yield untagged B, C and RRE proteins that can catalyze lasso peptide formation in the periplasm.

To produce the M13 phage library displaying lasso peptides, the ssPelB-fusilassin A*-TEV-p3 phagemid library and the ssTorA-B/ssTorA-C/ssTorA-RRE plasmid are first transformed into E. coli SS320 (Lucigen, Cat #60512-1) via electroporation following the manufacturer's instructions. The E. coli SS320 strain contains the tetracycline resistance gene as a selection marker. Following transformation, the E. coli cells are recovered in 1 mL of 2×YT medium for 1 hour at 37° C. in an incubator shaker at 250 rpm. After one-hour incubation, the culture is spread on 2×YT agar containing 100 μg/mL ampicillin, 25 μg/mL chloramphenicol, and 10 μg/mL tetracycline. The 2×YT agar plate is incubated overnight at 37° C. to yield single colonies. The next day, the colonies, consisting of 3× coverage of the library size, from the overnight agar plate are harvested and used to prepare a 5 mL overnight culture in 2×YT containing 2% (w/v) glucose, 100 μg/mL ampicillin, 25 μg/mL chloramphenicol, and 10 μg/mL tetracycline. This overnight culture is subsequently used to inoculate a fresh culture of 2×YT at 1% v/v (1 mL/100 mL) containing 2% (w/v) glucose and the same antibiotics. The freshly inoculated culture is grown at 37° C. in an incubator shaker at 250 rpm for 4 to 5 hours with OD₆₀₀ monitored every 30 minutes. When the culture reaches mid-log phase (OD₆₀₀=0.4-0.5), helper phage M13K07 stock at 10¹² pfu/mL is added to the culture at a ratio of 1:500 (v/v) helper phage:culture media. After addition of helper phage, the culture is further incubated at 37° C. in an incubator shaker at 250 rpm for 1 hour to allow phage transfection. Following the one-hour incubation, kanamycin is added at 60 μg/mL to remove any uninfected E. coli cells. To initiate phage production, the expression of ssPelB-fusilassin A*-TEV-p3, ssTorA-B, ssTorA-C and ssTorA-RRE is induced with IPTG at 1 mM. The induced culture is then incubated at 28° C. in an incubator shaker at 250 rpm for 24 hours to produce phage. During the phage assembly, the simultaneous presence of two to three copies of the wild-type p3 coat protein (encoded by the helper phage) facilitates efficient assembly of infective phage. As the result, each lasso peptide-TEV-p3 fusion protein is displayed at two to three copies per phage particle.

Following the production of phage, the E. coli cells are removed by two successive centrifugation steps (14,000×g, 15 min, 4° C.). The upper 80% of the supernatant is collected and mixed with one-fourth volume of polyethylene glycol 8000 (PEG 8000)/NaCl solution (20% PEG 8000, 2.5 M NaCl). The thoroughly mixed sample is placed on ice overnight to precipitate the phage. After overnight incubation on ice, the phage is pelleted by centrifugation at 11,000×g for 10 minutes at 4° C. The supernatant is discarded, and the pellet is resuspended in 2 mL of PBS buffer (pH=7.4). The resuspended sample is then centrifuged again at 14,000×g for 15 minutes at 4° C. to pellet insoluble debris. After precipitation of insoluble debris, the supernatant is transferred to a fresh tube and the phage is precipitated for the second time by adding one-fourth volume of polyethylene glycol 8000 (PEG 8000)/NaCl solution (20% PEG 8000, 2.5 M NaCl). The sample is then thoroughly mixed and placed on ice for at least two hours. The phage is again pelleted by centrifugation at 11,000×g for 10 minutes at 4° C. The supernatant is discarded, and the pelleted phage is resuspended in 500 mL of PBS buffer (pH=7.4). The concentration of the phage is determined by UV absorbance as described by Day and Wiseman (The Single-Stranded DNA Phages, Cold Spring Harbor, N.Y., 1978, p 605): phage concentration (phages/mL)=((A₂₆₉−A₃₂₀)×6×10¹⁶)/(phage genome size in nt)×dilution factor. The resuspended phage supernatant is passed through a 0.22 μm filter for sterilization.

To detect display of wild-type and mutant fusilassin lasso peptides on the mature phage, the filtered M13 phage library is diluted and used to infect E. coli cells on soft agar to obtain individual plagues derived from single-phage infection. Ten isolated plaques are individually cultured in 2YT media containing 2% (w/v) glucose and the same antibiotics at 28° C. for 16 hours and subjected to the phage purification procedure as described in the previous paragraph to obtain purified individual phage variants. The purified phage variant samples are individually treated with TEV protease (Sigma Cat. #T4455) to release wild-type and mutant fusilassin lasso peptides following the manufacturer's instructions. The protease digestion reactions are then treated with an equal volume of methanol, thoroughly mixed and centrifuged to precipitate insoluble debris. The soluble factions which contain released wild-type and mutant fusilassin lasso peptides fused to Linker 1 and part of TEV protease recognition site (fusilassin-Linker 1-Glu-Asn-Leu-Tyr-Phe-Gln) are concentrated and subjected to MALDT-TOF MS analysis. The presence of ssPelB-fusilassin A*-TEV-p3 DNA sequences in the mature phage is also independently detected by PCR amplification and DNA sequencing.

### 6.6 Example 6: Directed Evolution of a Single Lasso Peptide to Produce High-Affinity Ligands Via Whole Cell Panning Using M13 Phage Display

This example describes methods for directed evolution of a single lasso peptide to produce high-affinity ligands of glucagon receptor (GCGR) via whole cell panning using M13 phage display.

To evolve a lasso peptide to become a high-affinity antagonist of glucagon receptor (GCGR), BI-32169 (Gly-Leu-Pro-Trp-Gly-Cys-Pro-Ser-Asp-Ile-Pro-Gly-Trp-Asn-Thr-Pro-Trp-Ala-Cys) (SEQ ID NO:2636) discovered in Streptomyces sp. (Streicher et al., J. Nat. Prod. 2004, 67, 1528-1531) is chosen as a starting scaffold for evolution. Since the sequence of peptidase (B), cyclase (C) and RRE of BI-32169 have not been identified, peptidase (B), cyclase (C) and RRE of a BI-32169 analog (Gly-Leu-Pro-Trp-Gly-Cys-Pro-Asn-Asp-Leu-Phe-Phe-Val-Asn-Thr-Pro-Phe-Ala-Cys) (SEQ ID NO: 2637) identified in Kibdelosporangium sp. MJ126-NF4 are used to construct the ssTorA-B/ssTorA-C/ssTorA-RRE plasmid. Pavlova et al. (J. Biol. Chem. 2008, 283:25589-95) have shown that lasso peptide processing enzymes B, C and RRE recognize the leader peptide of a lasso precursor peptide and exhibit plasticity toward the core peptide. Moreover, the amino acid sequence of the core peptide can be altered to include mutations, deletions and C terminal extension (Pan and Link. J. Am. Chem. Soc. 2011, 133:5016-23; Zong et al. ACS Chem. Biol. 2016, 11:61-8). Therefore, the leader peptide sequence of BI-32169 is replaced with the leader peptide sequence of the BI-32169 analog to construction the hybrid BI-32169 precursor peptide A (Met-Ile-Lys-Asp-Asp-Glu-Ile-Tyr-Glu-Val-Pro-Thr-Leu-Val-Glu-Val-Gly-Asp-Phe-Ala-Glu-Leu-Thr-Leu-Gly-Leu-Pro-Trp-Gly-Cys-Pro-Ser- Asp-Ile-Pro-Gly-Trp-Asn-Thr-Pro-Trp-Ala-Cys) (SEQ ID NO: 2639) so that this hybrid precursor peptide A can be processed by the BI-32169 analog processing enzymes B, C and RRE from Kibdelosporangium sp. MJ126-NF4 for formation of BI-32169 lasso peptide. Leveraging the plasticity of lasso peptide processing enzymes, individual NNK phage libraries per mutated amino acid position are generated following the procedures described in Example 5.

To select for antagonists of glucagon receptor (GCGR), the individual NNK phage libraries are screened for their ability to bind GCGR expressed on the surface of CHO-S cells (Life Technologies) in the presence of glucagon (GCG). Following a similar procedure to the whole cell panning method reported by Jones et al., Sci Rep. 2016, 18; 6:26240, the CHO-S cells expressing GCGR are first washed in PBS, then blocked in 5 mL 2% (w/v) milk-PBS (MPBS) with rotation for 30 minutes at 4° C. Approximately, 10¹² phage particles from the phage library stock are also blocked in MPBS. The blocked phage particles are then added to the blocked cells and incubated with rotation for 1 hour at 4° C. in the presence of glucagon. The cells are then washed three times using Wash Buffer (PBS, 0.1% (v/v) Tween-20, pH 5.0), followed by 3 washes with PBS (pH 7.4) to remove unbound phage particles. The bound phage particles are eluted from the cells by incubating the cells in Elution Buffer (75 mM Citrate, pH 2.3) for 6 minutes at room temperature. After centrifugation at 800×g for 5 minutes, the supernatant is neutralized with 1 M Tris (pH 7.5). The neutralized phage eluate is used to infect E. coli SS320 cells transformed with the ssTorA-B/ssTorA-C/ssTorA-RRE plasmid. Phage particles are then prepared for subsequent rounds of phage panning by using M13K07 helper phage. After the first round of phage panning, the phagemid DNA is amplified for DNA sequencing analysis to reveal the amino acids mutations and positions that are beneficial in antagonizing GCG-GCGR binding. These beneficial mutations and positions are then incorporated into the design of a combinatorial phagemid library for next round of sequence selection. Such sequence selection via phage panning can be continued for several rounds with the sequence diversity monitored by DNA sequencing after each round of selection. To evolve for high-affinity antagonists of GCGR, the screening parameters and the composition of binding and washing media, such as incubation time, temperature, pH, salts and detergents, are adjusted to select for antagonists with increased binding affinity. The resulting high-affinity BI32169 mutants are further examined individually for their ability to inhibit calcium influx induced by GCG-GCGR binding using FLIPR® Calcium Assay (Molecular Devices, Cat. #FLIPR Calcium 6) with Ready-to-Assay™ Glucagon Receptor Frozen Cells (EMD Millipore, Cat. #HTS112RTA).

### 6.7 Example 7: In Vitro Selection and Evolution of a Lasso Peptide Library to Enrich High-Affinity Ligands Via Whole Cell Panning Using M13 Phage Display

The example describes methods of in vitro selection and evolution of a lasso peptide library to enrich high-affinity ligands of glucagon receptor (GCGR) via whole cell panning using M13 phage display.

To screen for high-affinity antagonists of glucagon receptor (GCGR) using M13 phage display, a phage library is designed to display lasso peptides with the size of the ring ranging from 7, 8 to 9 amino acid residues and each of the core peptide residues mutated, except for the residue(s) for the ring formation. To produce this phage library, the fusilassin precursor peptide A (Met-Glu-Lys-Lys-Lys-Tyr-Thr-Ala-Pro-Gln-Leu-Ala-Lys-Val-Gly-Glu-Phe-Lys-Glu-Ala-Thr-Gly↓Trp-Tyr-Thr-Ala-Glu-Trp-Gly-Leu-Glu-Leu-Ile-Phe-Val-Phe-Pro-Arg-Phe-Ile) (SEQ ID NO: 2632) is chosen as a starting sequence and follow the procedures described in Examples 5 and 6 to replace the fusilassin core peptide sequence (Trp-Tyr-Thr-Ala-Glu-Trp-Gly-Leu-Glu-Leu-Ile-Phe-Val-Phe-Pro-Arg-Phe-Ile)(SEQ ID NO: 2631) with one of the following coding sequences NNK-NNK-NNK-NNK-NNK-NNK-Glu-NNK-NNK-NNK-NNK-NNK-NNK-NNK-NNK-NNK-NNK-NNK-NNK (7-member ring), NNK-NNK-NNK-NNK-NNK-NNK-NNK-Glu-NNK-NNK-NNK-NNK-NNK-NNK-NNK-NNK-NNK-NNK-NNK (8-member ring), or NNK-NNK-NNK-NNK-NNK-NNK-NNK-NNK-Glu-NNK-NNK-NNK-NNK-NNK-NNK-NNK-NNK-NNK-NNK (9-member ring). Each of these coding sequences are synthesized as a pool of oligonucleotides by Twist Bioscience, Corp. and cloned into the modified pComb3 vector followed by the procedures described in Example 5 to produce a large phage library displaying diverse lasso peptides.

To select for antagonists of glucagon receptor (GCGR), the phage library is screened for their ability to bind GCGR expressed on the surface of CHO-S cells (Life Technologies) in the presence of glucagon (GCG). Following a similar procedure to the whole cell panning method reported by Jones et al., Sci Rep. 2016, 18; 6:26240, the CHO-S cells expressing GCGR are first washed in PBS, then blocked in 5 mL 2% (w/v) milk-PBS (MPBS) with rotation for 30 minutes at 4° C. Approximately, 10¹² phage particles from the phage library stock are also blocked in MPBS. The blocked phage particles are then added to the blocked cells and incubated with rotation for 1 hour at 4° C. in the presence of glucagon. The cells are then washed three times using Wash Buffer (PBS, 0.1% (v/v) Tween-20, pH 5.0), followed by 3 washes with PBS (pH 7.4) to remove unbound phage particles. The bound phage particles are eluted from the cells by incubating the cells in Elution Buffer (75 mM Citrate, pH 2.3) for 6 min at room temperature. After centrifugation at 800 g for 5 minutes, the supernatant is neutralized with 1M Tris (pH 7.5). The neutralized phage eluate is used to infect E. coli SS320 cells transformed with the ssTorA-B/ssTorA-C/ssTorA-RRE plasmid. Phage particles are then prepared for subsequent rounds of phage panning by using M13K07 helper phage. During each round of phage panning, a subpopulation of the phage library is enriched, and the sequence diversity of lasso peptides is monitored by Illumina Next-Gen DNA sequencing. To select for high-affinity antagonists of GCGR, the screening parameters and the composition of binding and washing media, such as incubation time, temperature, pH, salts and detergents, are adjusted to select for antagonists with increased binding affinity. The resulting high-affinity lasso peptides are further examined individually for their ability to inhibit calcium influx induced by GCG-GCGR binding using FLIPR® Calcium Assay (Molecular Devices, Cat. #FLIPR Calcium 6) with Ready-to-Assay™ Glucagon Receptor Frozen Cells (EMD Millipore, Cat. #HTS112RTA).

### 6.8 Example 8: In Vitro Selection and Evolution of a Phage-Display Lasso Peptide Library to Enrich High-Affinity Ligands Targeting Different Binding Pockets of Programmed Cell Death Protein-1 (PD-1)

The example describes methods for in vitro selection and evolution of a phage-display lasso peptide library to enrich high-affinity ligands targeting different binding pockets of programmed cell death protein-1 (PD-1).

Inhibition of T-cell immune checkpoints is one of the survival mechanisms that cancer cells elicit to evade the surveillance of the immune system. Among currently known immune checkpoint molecules, programmed cell death protein 1 (PD-1) has attracted much attention from researchers in the immune oncology field in the recent years. The successful development of monoclonal antibodies against PD-1 for treating cancers is typified by nivolumab (Opdivo) and pembrolizumab (Keytruda). At the molecular level, nivolumab and pembrolizumab recognize different epitopes, also known as “binding pockets,” of PD-1; while nivolumab binds the N-loop of PD-1 (Kd=3.06 pM), pembrolizumab targets the CD loop of PD-1 (Kd=29 pM) (Fessas et al. Seminars in Oncology. 2017, 44:136-140).

To screen and evolve lasso peptides for high affinity ligands targeting different binding pockets of PD-1, a phage-display lasso peptide library is generated following the procedure descried in Example 7. The generated lasso peptide library is then used to target immobilized recombinant PD-1 protein in the presence of recombinant PD-Li (programmed death ligand 1, a native PD-1 ligand), nivolumab or pembrolizumab. Such selection strategies apply directed evolution forces to yield ligands targeting three distinct binding pockets of PD-1 that are separately occupied by PD-L1, nivolumab and pembrolizumab.

To carry out an in vitro bio-panning, the recombinant human PD-1/Fc chimera protein is purchased from R&D Systems (Cat. #1086-PD) and immobilized on a Protein A coated plate (ThermoFisher, Cat. #15155) following the manufacturer's instruction. The uncoated surface of the plate is blocked with SuperBlock (PBS) blocking buffer (ThermoFisher, Cat. #37515) in the presence of 5% bovine serum albumin (BSA). The SuperBlock blocking buffer is removed and replaced with PBS buffer (10 mM bicarbonate phosphate buffer pH 7.4 and 150 mM NaCl). Approximately, 10¹² phage particles from the phage library stock are also blocked in 2% (w/v) milk-PBS (MPBS). The blocked phage particles are then added to the immobilized PD-1 protein on the plate in the presence of PD-L1, nivolumab or pembrolizumab. The plate is incubated for 1 hour at 4° C. and then washed three times using Wash Buffer (PBS, 0.1% (v/v) Tween-20, pH 5.0), followed by 3 washes with PBS (pH 7.4) to remove unbound phage particles. The bound phage particles are eluted from the cells by incubating the cells in Elution Buffer (75 mM Citrate, pH 2.3) for 6 min at room temperature. After centrifugation at 800 g for 5 minutes, the supernatant is neutralized with IM Tris (pH 7.5). The neutralized phage eluate is used to infect E. coli SS320 cells transformed with the ssTorA-B/ssTorA-C/ssTorA-RRE plasmid. Phage particles are then prepared for subsequent rounds of phage panning by using M13K07 helper phage. During each round of phage panning, a subpopulation of the phage library is enriched, and the sequence diversity of lasso peptides is monitored by Illumina Next-Gen DNA sequencing.

To evolve for high-affinity ligands of PD-1, the screening parameters and the composition of binding and washing media, such as incubation time, temperature, pH, salts and detergents, are adjusted to select for ligands with increased binding affinity. The resulting high-affinity lasso peptides are further examined individually for their ability to specifically block the binding of PD-L1, nivolumab or pembrolizumab to PD-1. The Kd values are obtained from a dose-response curve with ELISA using anti-SBP-tag mouse monoclonal antibody (EMD Millipore, Cat. #MAB 10764) and goat anti-mouse IgG antibody labeled with Alexa Fluor 488 (Abeam, Cat. #ab150077).

### 6.9 Example 9: Making a Phage-Display Lasso Peptide Library from Multiple Lasso Peptide Biosynthetic Gene Clusters

This example describes the methods for production of a phage-display lasso peptide library from multiple lasso peptide biosynthetic gene clusters (BGCs).

To produce a phage-display lasso peptide library from multiple lasso peptide biosynthetic gene clusters (BGCs), the DNA coding sequences for lasso peptide precursor (A), peptidase (B), cyclase (C) and Ripp Recognition Element (RRE) from each BGC are codon-optimized, synthesized and used for the construction of the two recombinant DNA plasmids per BGC: the ssPelB-lasso peptide precursor A-TEV-p3 phagemid shown in FIG. 4 and the MBP-B/MBP-C/MBP-RRE plasmid as shown in FIG. 5.

Following the procedure described in Example 4, each lasso peptide member of the phage-display library is individually generated with lasso formation catalyzed by purified peptidase (B), cyclase (C) and RRE from the respective BGC. For example, fusilassin precursor peptide A, displayed on the phage particle, is converted to fusilassin lasso peptide by purified MBP-fusilassin B, MBP-fusilassin C and MBP-fusilassin RRE; the BI-32169 analog precursor peptide A, displayed on the phage particle, is converted to the BI-32169 analog lasso peptide by purified MBP-the BI-32169 analog B, MBP-the BI-32169 analog C and MBP-the BI-32169 analog RRE; capistruin precursor peptide A, displayed on the phage particle, is converted to capistruin lasso peptide by purified MBP-capistruin B, and MBP-capistruin C.

The formation of lasso conformation is detected by MALDT-TOF MS analysis as described in Example 4. Upon formation of lasso peptides on the phage particles, the individual lasso peptide members are either pooled to create a phage-display lasso peptide library or individually deposited in the separate wells of a 96-well plate to create an arrayed phage-display lasso peptide library.

TABLE B
The list of protein sequences described
in the following Examples 10-14.

| SEQ | | | GenBank |
|---|---|---|---|
| ID | | | Accession |
| NO: | Name | A.A. sequence | # |
| 2659 | HOC | MTFTVDITPKTPTGVIDETK | NP_ |
| | (T4 phage) | QFTATPSGQTGGGTITYAWS | 049793 |
| | | VDNVPQDGAEATFSYVLKGP | |
| | | AGQKTIKVVATNTLSEGGPE | |
| | | TAEATTTITVKNKTQTTTLA | |
| | | VTPASPAAGVIGTPVQFTAA | |
| | | LASQPDGASATYQWYVDDSQ | |
| | | VGGETNSTFSYTPTTSGVKR | |
| | | IKCVAQVTATDYDALSVTSN | |
| | | EVSLTVNKKTMNPQVTLTPP | |
| | | SINVQQDASATFTANVTGAP | |
| | | EEAQITYSWKKDSSPVEGST | |
| | | NVYTVDTSSVGSQTIEVTAT | |
| | | VTAADYNPVTVTKTGNVTVT | |
| | | AKVAPEPEGELPYVHPLPHR | |
| | | SSAYIWCGWWVMDEIQKMTE | |
| | | EGKDWKTDDPDSKYYLHRYT | |
| | | LQKMMKDYPEVDVQESRNGY | |
| | | IIHKTALETGIIYTYP | |
| 2660 | SOC | MASTRGYVNIKTFEQKLDGN | NP_ |
| | (T4 phage) | KKIEGKEISVAFPLYSDVHK | 049644 |
| | | ISGAHYQTFPSEKAAYSTVY | |
| | | EENQRTEWIAANEDLWKVTG | |

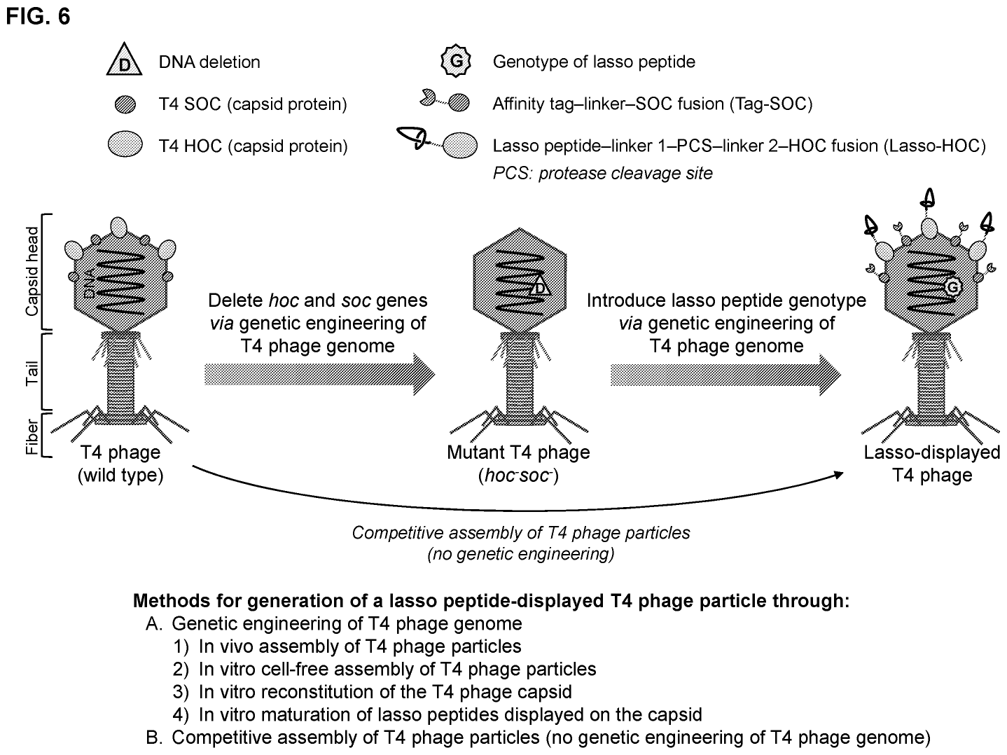

T4 phage is a large double-stranded DNA virus that infects E. coli. The phage particle consists of a capsid head and a tail with a sheath terminating in a base plate to which six tail fibers are attached. The 168 kb DNA genome of T4 phage is packed into the capsid head during the assembly of phage particles (Miller E S. et al., Microbiol Mol Biol Rev. 2003, 67(1):86-156). Unlike filamentous phages (e.g. M13 phage) that require periplasmic secretion of coat proteins for assembly of progeny phage particles, T4 phage, an archetype of lytic phages, assembles the progeny phage particles in the cytoplasm of the bacterial host cell. Therefore, lytic phages, such as T4, T7, lambda (λ), phi X 174 (ϕX 174) and MS2, do not require periplasmic secretion of phage coat proteins. Instead, the T4 progeny phages are released from the cytoplasm by lysis of the bacterial cell wall at the late stage of the lytic infection cycle (Bazan et al., Hum Vaccin Immunother. 2012, 8(12):1817-28). Furthermore, recent studies demonstrated that lytic phages, such as T4, T7, phi X 174 (ϕX 174) and MS2, can be entirely synthesized from their genome in one pot reactions using an E. coli, cell-free TX-TL system (Shin J. et al., ACS Synth Biol. 2012, 1(9):408-13; Rustad M. et al., J Vis Exp. 2017, (126); Rustad M. et al., Synthetic Biology, Volume 3, Issue 1, 1 Jan. 2018, ysy002). Since the discovery of T4 phage in the 1940s, several genetic engineering methods have been developed to enable manipulation of T4 phage genome. These methods include phage genetic cross, DNA homologous recombination, DNA recombineering, CRISPR-Cas-mediated genetic engineering, genome fragment ligation, and de novo phage genome assembly (Pires et al., Microbiol Mol Biol Rev. 2016, 80(3):523-43). Such genetic engineering tools have aided the development of several display systems based on T4, T7, or lambda (λ) phages for molecular evolution, such as affinity maturation of monoclonal antibodies and receptor ligands (Bazan et al., Hum Vaccin Immunother. 2012, 8(12):1817-28; Szardenings et al., J Biol Chem. 1997, 272(44):27943-8; Jiang et al., Infect Immun. 1997, 65(11):4770-7; Burgoon et al., J Immunol. 2001, 167(10):6009-14; Sternberg N. and Hoess R H., Proc Natl Acad Sci USA. 1995, 92(5):1609-13). The examples provided below utilize T4 phage HOC (highly immunogenic outer capsid) protein to display a lasso peptide fused to the N terminus of HOC protein on the surface of the T4 capsid (Jiang et al., Infect Immun. 1997, 65(11):4770-7) (FIG. 6). To further isolate or enrich the lasso peptide-displayed phage particles with affinity chromatography, T4 phage SOC (small outer capsid) protein is also manipulated to display an affinity tag fused to the N-terminus of SOC protein (Li Q. et al., J Mol Biol. 2006, 363(2):577-88; Ceglarek et al., Sci Rep. 2013, 3:3220; Da̧browska K. et al., Methods Mol Biol., 1898:81-87). T4 HOC and SOC are non-essential capsid protein that exhibits high-affinity binding capability to the core capsid. Several studies demonstrated that T4 HOC and SOC can be assembled onto the capsid either during in vivo phage particle assembly (Jiang et al., Infect Immun. 1997, 65(11):4770-7; Ren Z. and Black L W., Gene. 1998, 215(2):439-44) or through in vitro reconstitution of the capsid (Shivachandra S B. Et al., Virology. 2006, 345(1):190-8; Li Q. et al., J Mol Biol. 2007, 370(5):1006-19). Thus, a lasso peptide fused to HOC or SOC can be displayed on the T4 phage capsid: (1) during in vivo assembly of T4 phage particles in an E. coli cell (Example 10), (2) during in vitro assembly of T4 phage particles in a cell-free system (Example 11), (3) by in vitro reconstitution of the T4 phage capsid (Example 12), (4) by in vitro maturation of lasso peptides displayed on the capsid (Example 13), or (5) via competitive assembly of T4 phage particles (Example 14).

### 6.10 Example 10: In Vivo Assembly of T4 Phage Particles in an E. coli Cell

This example describes the process for making T4 phage having a single lasso peptide fused to the T4 HOC protein, wherein the lasso peptide is formed during in vivo assembly of T4 phage particles in the cytoplasm of an E. coli cell as shown in FIG. 7.

The wild type T4 phage (ATCC 11303-B4) and E. coli strain B (ATCC 11303) are purchased from ATCC. The mutant T4 phage lacking the hoc and soc gene (hoc⁻soc⁻) is created from the wild type T4 phage by deleting hoc and soc genes with homologous recombination while simultaneously inserting an IPTG inducible E. coli promoter (e.g., pA1). The E. coli strain B is engineered to express lambda (λ) recombinase αβγ enzymes that enable efficient homologous recombination between T4 phage genome and a transformed plasmid vector. Prior to the infection of the mutant T4 phage (hoc⁻soc⁻), the engineered E. coli strain B is first transformed with the plasmid encoding lasso peptide biosynthesis enzymes fused to a maltose-binding protein (MBP-B, MBP-C and MBP-RRE), and subsequently with the second plasmid encoding the protein for lasso precursor peptide-HOC (preLasso-HOC) fusion and the protein for affinity tag-SOC (Tag-SOC) fusion. The double-transformed E. coli cells are then infected with the mutant T4 phage (hoc⁻soc⁻). Following the infection, the parent T4 phage genome (hoc⁻soc⁻) is inserted into the cytoplasm of the E. coli cell, recombined with the lasso-hoc/tag-hoc plasmid, and replicated to produce multiple copies of progeny phage genome that carries the recombined lasso-hoc/tag-hoc coding sequence. From the progeny phage genome, the expression of the recombined lasso-hoc and tag-soc coding sequences is under the control of the pA1 promoter previously inserted next to the site of homologous recombination. During the synthesis of phage structural proteins, the preLasso-HOC fusion protein is simultaneously expressed upon the IPTG induction. Once expressed, the lasso precursor peptide portion of the preLasso-HOC fusion protein is further processed into a mature lasso peptide as a Lasso-HOC fusion protein. During the assembly of T4 progeny phage particles, Lasso-HOC and Tag-SOC are incorporated into the capsid. At the late stage of the lytic infection cycle, the lasso-displayed T4 progeny phage particles are released into the culture media by lysis of the bacterial cell wall.

The plasmid encoding MBP-B, MBP-C and MBP-RRE is constructed similarly to the ssTorA-B/ssTorA-C/ssTorA-RRE plasmid described in Example 1 by replacing the ssTorA sequence with the sequence encoding the truncated maltose binding protein (MBP) devoid of the secretion sequence residues 2-29. The lasso-hoc/tag-soc plasmid is constructed by cloning the sequence encoding the fusilassin precursor peptide-HOC (fusilassin-HOC) fusion protein and the sequence encoding the six-histidine tag-SOC (6×His-SOC) fusion protein into a cloning (non-expression) vector. The presence of the two 250 bp DNA homology arms in the cloning vector allows insertion of the cloned sequence into the mutant T4 phage genome at the designated recombination site. Following transformation of the two plasmids, the double-transformed E. coli cells are incubated at 37° C. for 18 hours (overnight) under the selection of appropriate antibiotics. The overnight culture is then diluted at 1:100 in LB media and further incubated at 37° C. to reach the exponential growth phase (OD600 of 0.2 to 0.4). This fresh E. coli culture is then infected with the mutant T4 phage (hoc⁻soc⁻) at the multiplicity of infection (MOI) of 10 in the presence of 0.5 mM IPTG to induce expression of fusilassin-HOC and 6×His-SOC. Following the infection, the culture is incubated at 37° C. for 5 to 6 hours until cell lysis occurs. The cell lysate containing the phage particles is cleared of cellular debris by centrifugation at 5,000×g for 30 minutes at 4° C. The resulting supernatant is then filtered through a vacuum-driven filtration system with 0.2 μm pore size (Stericup, Millipore). If the cell lysis is incomplete, PEG precipitation and chloroform extraction may be necessary prior to the filtration step. Following the filtration step, the recombinant T4 phage particles in the filtered supernatant are isolated with affinity chromatography using Ni-NTA resin (QIAGEN) as described by Ceglarek et al. (Sci Rep. 2013, 3:3220). Optionally, the isolated recombinant T4 phage particles can be further purified using sucrose gradient centrifugation or chromatography.

### 6.11 Example 11: In Vitro Assembly of T4 Phage Particles in a Cell-Free System

This example describes the process for making T4 phage having a single lasso peptide fused to the T4 HOC protein, wherein the lasso peptide is formed during in vitro assembly of T4 phage particles in a cell-free system as shown in FIG. 8.

The wild type T4 phage (ATCC 11303-B4) and E. coli strain B (ATCC 11303) are purchased from ATCC. The mutant T4 phage lacking the hoc and soc gene (hoc⁻soc⁻) is created from the wild type T4 phage by deleting hoc and soc genes with homologous recombination while simultaneously inserting an IPTG inducible E. coli promoter (e.g., pA1). The T4 phage genomic DNA is extracted as described by Rustad M. et al. (Synthetic Biology, Volume 3, Issue 1, 1 Jan. 2018, ysy002). The E. coli strain B is engineered to express lambda (λ) recombinase αβγ enzymes that enable efficient homologous recombination between T4 phage genome and an added plasmid vector. The cell extracts of the engineering E. coli strain B and the energy buffer are prepared as described by Sun et al. (J Vis Exp. 2013, (79):e50762) and Rustad M. et al. (Synthetic Biology, Volume 3, Issue 1, 1 Jan. 2018, ysy002). The MBP-B/MBP-C/MBP-RRE plasmid and the Fusilassin-HOC/6×His-SOC plasmid are constructed as described in Example 10.

To produce the fusilassin-displayed T4 phage, the genomic DNA of mutant T4 phage (hoc⁻soc⁻) is added at 1 nM into 40 μL of the cell-free reaction containing 33% of the cell extracts and 66% of the energy buffer. Simultaneously, the MBP-B/MBP-C/MBP-RRE plasmid is added at 20 nM and the fusilassin-HOC/6×His-SOC plasmid is added at 10 nM. Upon the addition of IPTG at 0.5 mM, the cell-flee reaction mixture is incubated at 29° C. for 10-12 hours. During the incubation, the added T4 phage genome is recombined with the fusilassin-HOC/6×His-SOC plasmid and replicated to produce multiple copies of progeny phage genome that carries the recombined fusilassin-HOC/6×His-SOC coding sequence. From the progeny phage genome, the expression of the recombined fusilassin-HOC and 6×His-SOC coding sequences is under the control of the pA1 promoter previously inserted next to the site of homologous recombination. During the synthesis of phage structural proteins, the fusilassin precursor peptide-HOC fusion protein is also expressed upon the IPTG induction. Once expressed, the fusilassin precursor peptide is further processed into a mature lasso peptide. During the assembly of T4 progeny phage particles, fusilassin-HOC and 6×His-SOC are incorporated into the capsid to produce the fusilassin-displayed T4 phage particles in the reaction mixture.

The cell-flee reaction mixture containing the phage particles is cleared of cellular debris by centrifugation at 5,000×g for 30 minutes at 4° C. The supernatant is further cleared by chloroform extraction and then filtered through a vacuum-driven filtration system with 0.2 μm pore size (Stericup, Millipore). Following the filtration step, the recombinant T4 phage particles in the filtered supernatant are isolated with affinity chromatography using Ni-NTA resin (QIAGEN) as described by Ceglarek et al. (Sci Rep. 2013, 3:3220). Optionally, the isolated recombinant T4 phage particles can be further purified using sucrose gradient centrifugation or chromatography.

### 6.12 Example 12: In Vitro Reconstitution of the T4 Phage Capsid

This example describes the process for making T4 phage having a single lasso peptide fused to the T4 HOC protein, wherein the isolated lasso peptide-HOC fusion protein is reconstituted in vitro onto the T4 capsid lacking HOC (HOC) as shown in FIG. 9.

The wild type T4 phage (ATCC 11303-B4) and E. coli strain B (ATCC 11303) are purchased from ATCC. The mutant T4 phage lacking the hoc and soc gene (hoc⁻soc⁻) is created from the wild type T4 phage by deleting hoc and soc genes with homologous recombination. To propagate the mutant T4 phage (hoc⁻soc⁻), the phage particles are prepared in the absence of the MBP-B/MBP-C/MBP-RRE and the lasso-hoc/tag-soc plasmids by either in vivo assembly as described in Example 10 or in vitro cell-free assembly as described in Example 11. To facilitate affinity purification, a plasmid vector encoding the fusilassin-HOC-Strep fusion protein is created to expression the fusilassin-HOC protein fused to a C terminal Strep tag. Both the fusilassin-HOC-Strep and 6×His-SOC fusion proteins are expressed either in vivo (e.g., E. coli) or in vitro (e.g., in a cell-free system) and purified using Strep-Tactin resin (IBA Lifesciences) and Ni-NTA resin (QIAGEN), respectively. The in vitro assembly of fusilassin-HOC-Strep and 6×His-SOC onto the capsid of the mutant T4 phage (hoc⁻soc⁻) is carried out as described by Sathaliyawala et al. (J Virol. 2006, 80(15):7688-98). Briefly, 2×10¹⁰ PFU of isolated mutant T4 phage (hoc⁻soc⁻) are centrifuged at 13,000×g at 4° C. for an hour. The pellets are resuspended in 10 μL of buffer containing 50 mM phosphate buffer [pH 7.0], 75 mM NaCl, and 1 mM MgSO₄. Purified fusilassin-HOC-Strep and 6×His-SOC fusion proteins are added at the desired concentration in a total reaction mixture of 100 μL and incubated at 37° C. for 45 minutes. After the incubation, phages are precipitated by centrifugation at 13,000×g at 4° C. for an hour. The pellet is washed twice with 1 mL of the same buffer and transferred to a new tube or a new well of a 96-well plate. Optionally, the reconstituted T4 phage particles are further purified with affinity chromatography using Ni-NTA resin (QIAGEN) as described by Ceglarek et al. (Sci Rep. 2013, 3:3220).

Following the similar procedure in parallel, a phage display library is constructed to vary the amino acid composition of the lasso peptide displayed on the capsid. Each member of the phage display library is identified by tube ID number or well position plus plate ID number.

### 6.13 Example 13: In Vitro Maturation of Lasso Peptides Displayed on the Capsid

This example describes the process for making T4 phage having a single lasso peptide fused to the T4 HOC protein, wherein the lasso precursor peptide-HOC fusion protein, displayed on the T4 capsid, is processed in vitro by isolated lasso peptide biosynthesis enzymes as shown in FIG. 10.

The recombinant T4 phage (lasso-hoc/tag-soc) displaying fusilassin precursor peptide-HOC and 6×His-SOC fusion proteins is prepared in the absence of the MBP-B/MBP-C/MBP-RRE plasmid as described in Examples 10 and 11. The maturation of fusilassion is catalyze by the purified recombinant MBP-B, MBP-C and MBP-RRE proteins as described in Example 4 (FIG. 5). In this case, the amino acid composition of the lasso peptide (phenotype) displayed on the phage capsid is identified by the genotype of the phage.

Alternative, the in vitro reconstituted T4 phage (hoc⁻soc⁻) displaying fusilassin precursor peptide-HOC and 6×His-SOC fusion proteins is prepared as described in Example 12, except that the fusilassin precursor peptide-HOC-Strep fusion protein is not pre-processed by the lasso biosynthetic enzyme MBP-B, MBP-C and MBP-RRE. Instead, the maturation of fusilassion is catalyze by the purified recombinant MBP-B, MBP-C and MBP-RRE proteins as described in Example 4 (FIG. 5). In this case, the amino acid composition of the lasso peptide (phenotype) displayed on the phage capsid is identified by tube ID number or well position plus plate ID number.

### 6.14 Example 14: Competitive Phage Display

This example describes the process for making a competitive T4 phage display having a single lasso peptide fused to the T4 HOC protein, wherein the lasso precursor-HOC fusion protein is competing with unmodified HOC protein for assembly of T4 phage capsid as shown in FIGS. 11A and 11B.

Without insertion of the lasso peptide coding sequence into the T4 phage genome, the fusilassin-HOC and the 6×His-SOC fusion proteins are incorporated onto the capsid in the presence of wild type HOC and SOC proteins through a technique termed competitive phage display (Ceglarek et al., Sci Rep. 2013, 3:3220). The competitive T4 phage display is generated from one of the three following systems: (1) in vivo assembly as described in Example 10, except that wild type T4 phage is used to infect E. coli cells instead of the mutant T4 phage (hoc⁻soc⁻), (2) in vitro cell-free assembly as described in Example 11, except that wild type T4 phage genome is added into the cell extracts instead of the mutant T4 phage genome (hoc soc), and (3) in vitro reconstitution as described in Example 12, except that HOC and SOC are also presence in the mixture with the fusilassin-HOC-Strep and 6×His-SOC fusion proteins. In the case of competitive T4 phage display, the amino acid composition of the lasso peptide (phenotype) displayed on the phage capsid is identified by tube ID number or well position plus plate ID number.

### 7. SEQUENCES

Various exemplary amino acid and nucleic acid sequences are disclosed in this application, a summary of which are provided in the Summary Table. Additionally, Table 1 lists exemplary combinations of various components that can be used in connection with the present methods and systems. Table 2 lists example of lasso precursor and lasso core peptides. Table 3 lists examples of lasso peptidase. Table 4 lists examples of lasso cyclase. Table 5 lists examples of RREs.

TABLE 1
Summary Table

| Class | Description | Peptide No: # |
|---|---|---|
| A | Precursors | 1-1315 |
| B | Peptidase | 1316-2336 |
| C* | Cyclase | 2337-3761 |
| E** | RRE | 3762-4593 |
| CE | cyclase-RRE fusion | 2504 |
| CB | cyclase-peptidase fusion | 2903 |
| CE | cyclase-RRE fusion | 3608 |
| EB | RRE-peptidase fusion | 3768 |
| EB | RRE-peptidase fusion | 3770 |
| EB | RRE-peptidase fusion | 3793 |
| EB | RRE-peptidase fusion | 3811 |
| EB | RRE-peptidase fusion | 3818 |
| EB | RRE-peptidase fusion | 3851 |
| EB | RRE-peptidase fusion | 3855 |
| EB | RRE-peptidase fusion | 3887 |
| EB | RRE-peptidase fusion | 4004 |
| EB | RRE-peptidase fusion | 4018 |
| EB | RRE-peptidase fusion | 4045 |
| EB | RRE-peptidase fusion | 4076 |
| EB | RRE-peptidase fusion | 4132 |
| EB | RRE-peptidase fusion | 4150 |
| EB | RRE-peptidase fusion | 4167 |
| EB | RRE-peptidase fusion | 4168 |
| EB | RRE-peptidase fusion | 4225 |
| EB | RRE-peptidase fusion | 4262 |
| EB | RRE-peptidase fusion | 4379 |
| EB | RRE-peptidase fusion | 4414 |
| EB | RRE-peptidase fusion | 4499 |
| EB | RRE-peptidase fusion | 4504 |
| EB | RRE-peptidase fusion | 4507 |
| EB | RRE-peptidase fusion | 4512 |
| EB | RRE-peptidase fusion | 4517 |
| EB | RRE-peptidase fusion | 4518 |
| EB | RRE-peptidase fusion | 4529 |
| EB | RRE-peptidase fusion | 4532 |
| EB | RRE-peptidase fusion | 4542 |
| EB | RRE-peptidase fusion | 4559 |
| EB | RRE-peptidase fusion | 4561 |
| EB | RRE-peptidase fusion | 4562 |

*including CE and CB fusion sequences
**Including EB fusion sequences

TABLE 2
Exemplary Combinations of (i) Lasso Precursor Peptide;
(ii) Lasso Peptidase; (iii) Lasso Cyclase; (iv) RRE;
(v) Peptidase Fusion; and/or (vi) Cyclase Fusion

| Peptide No: #; GI#; | | | | | |
|---|---|---|---|---|---|
| Accession#; Nucleic | | | RRE | CE | EB |
| Acid SEQ ID NO: #; | | | Pep- | Pep- | Pep- |
| Amino Acid SEQ ID | Peptidase | Cyclase | tide | tide | tide |
| NO: #; Junction | Peptide | Peptide | No: | No: | No: |
| Position | No: # | No: # | # | # | # |
| 1; 167643973; | 1598 | 3360 | n/a | n/a | n/a |
| NC_010338.1; | | | | | |
| 1; 2; 22/23 | | | | | |
| 2; 167643973; | 1598 | 3360 | n/a | n/a | n/a |
| NC_010338.1; | | | | | |
| 3; 4; 21/22 | | | | | |
| 3; 167643973; | 1324 | 2349 | n/a | n/a | n/a |
| NC_010338.1; | | | | | |
| 5; 6; 21/22 | | | | | |
| 4; 167643973; | 1324 | 2349 | n/a | n/a | n/a |
| NC_010338.1; | | | | | |
| 7; 8; 22/23 | | | | | |
| 5; 737103862; | 1943 | 3191 | n/a | n/a | n/a |
| NZ_JQJP01000023.1; | | | | | |
| 9; 10; 21/22 | | | | | |
| 6; 737089868; | 1943 | 3191 | n/a | n/a | n/a |
| NZ_JQJN01000025.1; | | | | | |
| 11; 12; 21/22 | | | | | |
| 7; 737089868; | 1942 | 3190 | n/a | n/a | n/a |
| NZ_JQJN01000025.1; | | | | | |
| 13; 14; 21/22 | | | | | |
| 8; 737089868; | 1942 | 3190 | n/a | n/a | n/a |
| NZ_JQJN01000025.1; | | | | | |
| 15; 16; 21/22 | | | | | |
| 9; 930490730; | 2056 | 3614 | 4407 | n/a | n/a |
| NZ_LJCU01000014.1; | | | | | |
| 17; 18; 13/14 | | | | | |
| 10; 930490730; | 2279 | 3681 | 4541 | n/a | n/a |
| NZ_LJCU01000014.1; | | | | | |
| 19; 20; 13/14 | | | | | |
| 11; 657284919; | 1438 | 2500 | 3861 | n/a | n/a |
| JJMG01000143.1; | | | | | |
| 21; 22; 21/22 | | | | | |
| 12; 657284919; | 2114 | 3635 | 4459 | n/a | n/a |
| JJMG01000143.1; | | | | | |
| 23; 24; 21/22 | | | | | |
| 13; 657284919; | 1988 | 3570 | 4347 | n/a | n/a |
| JJMG01000143.1; | | | | | |
| 25; 26; 21/22 | | | | | |
| 14; 663380895; | n/a | 3091 | 4259 | n/a | n/a |
| NZ_JNZW01000001.1; | | | | | |
| 27; 28; 21/22 | | | | | |
| 15; 485035557; | 1566 | 3438 | n/a | n/a | n/a |
| NZ_AECN01000315.1; | | | | | |
| 29; 30; 28/29 | | | | | |
| 16; 485035557; | 1566 | 2971 | n/a | n/a | n/a |
| NZ_AECN01000315.1; | | | | | |
| 31; 32; 28/29 | | | | | |
| 17; 485035557; | 1566 | 2981 | n/a | n/a | n/a |
| NZ_AECN01000315.1; | | | | | |
| 33; 34; 28/29 | | | | | |
| 18; 485035557; | 1565 | 2970 | n/a | n/a | n/a |
| NZ_AECN01000315.1; | | | | | |
| 35; 36; 28/29 | | | | | |
| 19; 485035557; | 1318 | 2339 | n/a | n/a | n/a |
| NZ_AECN01000315.1; | | | | | |
| 37; 38; 28/29 | | | | | |
| 20; 485035557; | 1644 | 2772 | n/a | n/a | n/a |
| NZ_AECN01000315.1; | | | | | |
| 39; 40; 28/29 | | | | | |
| 21; 485035557; | 1533 | 3393 | n/a | n/a | n/a |
| NZ_AECN01000315.1; | | | | | |
| 41; 42; 28/29 | | | | | |
| 22; 485035557; | 1399 | 2451 | n/a | n/a | n/a |
| NZ_AECN01000315.1; | | | | | |
| 43; 44; 28/29 | | | | | |
| 23; 149147045; | 1571 | 3436 | n/a | n/a | n/a |
| NZ_ABBG01000168.1; | | | | | |
| 45; 46; 28/29 | | | | | |
| 24; 67639376; | 1525 | 3349 | n/a | n/a | n/a |
| NZ_AAHO01000116.1; | | | | | |
| 47; 48; 28/29 | | | | | |
| 25; 149147045; | 1570 | 3300 | n/a | n/a | n/a |
| NZ_ABBG01000168.1; | | | | | |
| 49; 50; 28/29 | | | | | |
| 26; 67639376; | 1523 | 2613 | n/a | n/a | n/a |
| NZ_AAHO01000116.1; | | | | | |
| 51; 52; 28/29 | | | | | |
| 27; 67639376; | 1525 | 3292 | n/a | n/a | n/a |
| NZ_AAHO01000116.1; | | | | | |
| 53; 54; 28/29 | | | | | |
| 28; 67639376; | 1523 | 3283 | n/a | n/a | n/a |
| NZ_AAHO01000116.1; | | | | | |
| 55; 56; 28/29 | | | | | |
| 29; 67639376; | 1526 | 3287 | n/a | n/a | n/a |
| NZ_AAHO01000116.1; | | | | | |
| 57; 58; 28/29 | | | | | |
| 30; 67639376; | 1525 | 2612 | n/a | n/a | n/a |
| NZ_AAHO01000116.1; | | | | | |
| 59; 60; 28/29 | | | | | |
| 31; 67639376; | 1525 | 3280 | n/a | n/a | n/a |
| NZ_AAHO01000116.1; | | | | | |
| 61; 62; 28/29 | | | | | |
| 32; 67639376; | 1526 | 3350 | n/a | n/a | n/a |
| NZ_AAHO01000116.1; | | | | | |
| 63; 64; 28/29 | | | | | |
| 33; 67639376; | 1525 | 3295 | n/a | n/a | n/a |
| NZ_AAHO01000116.1; | | | | | |
| 65; 66; 28/29 | | | | | |
| 34; 67639376; | 1525 | 3285 | n/a | n/a | n/a |
| NZ_AAHO01000116.1; | | | | | |
| 67; 68; 28/29 | | | | | |
| 35; 67639376; | 1523 | 3298 | n/a | n/a | n/a |
| NZ_AAHO01000116.1; | | | | | |
| 69; 70; 28/29 | | | | | |
| 36; 67639376; | 1526 | 3296 | n/a | n/a | n/a |
| NZ_AAHO01000116.1; | | | | | |
| 71; 72; 28/29 | | | | | |
| 37; 67639376; | 1525 | 3544 | n/a | n/a | n/a |
| NZ_AAHO01000116.1; | | | | | |
| 73; 74; 28/29 | | | | | |
| 38; 67639376; | 1526 | 3545 | n/a | n/a | n/a |
| NZ_AAHO01000116.1; | | | | | |
| 75; 76; 28/29 | | | | | |
| 39; 67639376; | 1524 | 2611 | n/a | n/a | n/a |
| NZ_AAHO01000116.1; | | | | | |
| 77; 78; 28/29 | | | | | |
| 40; 67639376; | 1523 | 2614 | n/a | n/a | n/a |
| NZ_AAHO01000116.1; | | | | | |
| 79; 80; 28/29 | | | | | |
| 41; 67639376; | 1526 | 3352 | n/a | n/a | n/a |
| NZ_AAHO01000116.1; | | | | | |
| 81; 82; 28/29 | | | | | |
| 42; 67639376; | 1525 | 3297 | n/a | n/a | n/a |
| NZ_AAHO01000116.1; | | | | | |
| 83; 84; 28/29 | | | | | |
| 43; 67639376; | 1525 | 3290 | n/a | n/a | n/a |
| NZ_AAHO01000116.1; | | | | | |
| 85; 86; 28/29 | | | | | |
| 44; 67639376; | 1396 | 2448 | n/a | n/a | n/a |
| NZ_AAHO01000116.1; | | | | | |
| 87; 88; 28/29 | | | | | |
| 45; 67639376; | 1523 | 3409 | n/a | n/a | n/a |
| NZ_AAHO01000116.1; | | | | | |
| 89; 90; 28/29 | | | | | |
| 46; 67639376; | 1525 | 3293 | n/a | n/a | n/a |
| NZ_AAHO01000116.1; | | | | | |
| 91; 92; 28/29 | | | | | |
| 47; 67639376; | 1526 | 3392 | n/a | n/a | n/a |
| NZ_AAHO01000116.1; | | | | | |
| 93; 94; 28/29 | | | | | |
| 48; 67639376; | 1525 | 3291 | n/a | n/a | n/a |
| NZ_AAHO01000116.1; | | | | | |
| 95; 96; 28/29 | | | | | |
| 49; 67639376; | 1525 | 2951 | n/a | n/a | n/a |
| NZ_AAHO01000116.1; | | | | | |
| 97; 98; 28/29 | | | | | |
| 50; 67639376; | 1525 | 3440 | n/a | n/a | n/a |
| NZ_AAHO01000116.1; | | | | | |
| 99; 100; 28/29 | | | | | |
| 51; 67639376; | 1997 | 3282 | n/a | n/a | n/a |
| NZ_AAHO01000116.1; | | | | | |
| 101; 102; 28/29 | | | | | |
| 52; 67639376; | 1526 | 2615 | n/a | n/a | n/a |
| NZ_AAHO01000116.1; | | | | | |
| 103; 104; 28/29 | | | | | |
| 53; 67639376; | 1395 | 2447 | n/a | n/a | n/a |
| NZ_AAHO01000116.1; | | | | | |
| 105; 106; 28/29 | | | | | |
| 54; 67639376; | 1523 | 2610 | n/a | n/a | n/a |
| NZ_AAHO01000116.1; | | | | | |
| 107; 108; 28/29 | | | | | |
| 55; 67639376; | 1523 | 3437 | n/a | n/a | n/a |
| NZ_AAHO01000116.1; | | | | | |
| 109; 110; 28/29 | | | | | |
| 56; 67639376; | 1526 | 3289 | n/a | n/a | n/a |
| NZ_AAHO01000116.1; | | | | | |
| 111; 112; 28/29 | | | | | |
| 57; 67639376; | 1523 | 3351 | n/a | n/a | n/a |
| NZ_AAHO01000116.1; | | | | | |
| 113; 114; 28/29 | | | | | |
| 58; 67639376; | 1525 | 3294 | n/a | n/a | n/a |
| NZ_AAHO01000116.1; | | | | | |
| 115; 116; 28/29 | | | | | |
| 59; 67639376; | 1526 | 3281 | n/a | n/a | n/a |
| NZ_AAHO01000116.1; | | | | | |
| 117; 118; 28/29 | | | | | |
| 60; 67639376; | 1317 | 2338 | n/a | n/a | n/a |
| NZ_AAHO01000116.1; | | | | | |
| 119; 120; 28/29 | | | | | |
| 61; 67639376; | 1525 | 3286 | n/a | n/a | n/a |
| NZ_AAHO01000116.1; | | | | | |
| 121; 122; 28/29 | | | | | |
| 62; 67639376; | 1526 | 2690 | n/a | n/a | n/a |
| NZ_AAHO01000116.1; | | | | | |
| 123; 124; 28/29 | | | | | |
| 63; 67639376; | 1447 | 2509 | n/a | n/a | n/a |
| NZ_AAHO01000116.1; | | | | | |
| 125; 126; 28/29 | | | | | |
| 64; 67639376; | 1404 | 2458 | n/a | n/a | n/a |
| NZ_AAHO01000116.1; | | | | | |
| 127; 128; 28/29 | | | | | |
| 65; 67639376; | 1526 | 3284 | n/a | n/a | n/a |
| NZ_AAHO01000116.1; | | | | | |
| 129; 130; 28/29 | | | | | |
| 66; 67639376; | n/a | 2511 | n/a | n/a | n/a |
| NZ_AAHO01000116.1; | | | | | |
| 131; 132; 28/29 | | | | | |
| 67; 67639376; | 1523 | 3383 | n/a | n/a | n/a |
| NZ_AAHO01000116.1; | | | | | |
| 133; 134; 28/29 | | | | | |
| 68; 740958729; | 1998 | 3288 | n/a | n/a | n/a |
| NZ_JPWT01000001.1; | | | | | |
| 135; 136; 28/29 | | | | | |
| 69; 485035557; | 1348 | 2380 | n/a | n/a | n/a |
| NZ_AECN01000315.1; | | | | | |
| 137; 138; 28/29 | | | | | |
| 70; 67639376; | 1520 | 2606 | n/a | n/a | n/a |
| NZ_AAHO01000116.1; | | | | | |
| 139; 140; 28/29 | | | | | |
| 71; 149147045; | 1571 | 2982 | n/a | n/a | n/a |
| NZ_ABBG01000168.1; | | | | | |
| 141; 142; 28/29 | | | | | |
| 72; 149147045; | 1570 | 3299 | n/a | n/a | n/a |
| NZ_ABBG01000168.1; | | | | | |
| 143; 144; 28/29 | | | | | |
| 73; 657295264; | n/a | 3465 | 4235 | n/a | n/a |
| NZ_AZSD01000040.1; | | | | | |
| 145; 146; 25/26 | | | | | |
| 74; 754788309; | 1695 | 2846 | 4184 | n/a | n/a |
| NZ_BBNO01000002.1; | | | | | |
| 147; 148; 29/30 | | | | | |
| 75; 928897585; | 2094 | 3458 | 4440 | n/a | n/a |
| NZ_LGKG01000196.1; | | | | | |
| 149; 150; 29/30 | | | | | |
| 76; 928897585; | 2271 | 3671 | 4537 | n/a | n/a |
| NZ_LGKG01000196.1; | | | | | |
| 151; 152; 29/30 | | | | | |
| 77; 754788309; | 2039 | 3370 | 4393 | n/a | n/a |
| NZ_BBNO01000002.1; | | | | | |
| 153; 154; 29/30 | | | | | |
| 78; 739918964; | 1901 | 3267 | 4494 | n/a | n/a |
| NZ_JJOH01000097.1; | | | | | |
| 155; 156; 29/30 | | | | | |
| 79; 928897585; | 1354 | 2386 | 3791 | n/a | n/a |
| NZ_LGKG01000196.1; | | | | | |
| 157; 158; 29/30 | | | | | |
| 80; 374982757; | 2058 | 3397 | 4029 | n/a | n/a |
| NC_016582.1; | | | | | |
| 159; 160; 13/14 | | | | | |
| 81; 374982757; | 2058 | 3397 | 4029 | n/a | n/a |
| NC_016582.1; | | | | | |
| 161; 162; 28/29 | | | | | |
| 82; 739918964; | 1901 | 3583 | 4295 | n/a | n/a |
| NZ_JJOH01000097.1; | | | | | |
| 163; 164; 29/30 | | | | | |
| 83; 852460626; | 1357 | 2392 | 3794 | n/a | n/a |
| CP011799.1; | | | | | |
| 165; 166; 29/30 | | | | | |
| 84; 514918665; | 1661 | 2797 | 4073 | n/a | n/a |
| NZ_AOPZ01000109.1; | | | | | |
| 167; 168; 32/33 | | | | | |
| 85; 396995461; | 2024 | 3338 | 3939 | n/a | n/a |
| AJGV01000085.1; | | | | | |
| 169; 170; 28/29 | | | | | |
| 86; 739830131; | n/a | 3259 | 4351 | n/a | n/a |
| NZ_JOJE01000039.1; | | | | | |
| 171; 172; 32/33 | | | | | |
| 87; 396995461; | 1400 | 2452 | 3833 | n/a | n/a |
| AJGV01000085.1; | | | | | |
| 173; 174; 28/29 | | | | | |
| 88; 374982757; | 1332 | 2357 | 3767 | n/a | 3768 |
| NC_016582.1; | | | | | |
| 175; 176; 13/14 | | | | | |
| 89; 374982757; | 1332 | 2357 | 3767 | n/a | 3768 |
| NC_016582.1; | | | | | |
| 177; 178; 28/29 | | | | | |
| 90; 664481891; | 2144 | 3121 | 4289 | n/a | n/a |
| NZ_JOJI01000011.1; | | | | | |
| 179; 180; 27/28 | | | | | |
| 91; 663732121; | n/a | 3094 | 4498 | n/a | n/a |
| NZ_JNZQ01000012.1; | | | | | |
| 181; 182; 22/23 | | | | | |
| 92; 742921760; | 1492 | 2571 | n/a | n/a | n/a |
| NZ_JWKL01000093.1; | | | | | |
| 183; 184; 37/38 | | | | | |
| 93; 742921760; | 1492 | 3303 | n/a | n/a | n/a |
| NZ_JWKL01000093.1; | | | | | |
| 185; 186; 37/38 | | | | | |
| 94; 389809081; | 2150 | 3328 | n/a | n/a | n/a |
| NZ_AJXW01000057.1; | | | | | |
| 187; 188; 26/27 | | | | | |
| 95; 389809081; | 1398 | 2450 | n/a | n/a | n/a |
| NZ_AJXW01000057.1; | | | | | |
| 189; 190; 26/27 | | | | | |
| 96; 655566937; | 1830 | 3056 | n/a | n/a | n/a |
| NZ_JAES01000046.1; | | | | | |
| 191; 192; 26/27 | | | | | |
| 97; 749673329; | 2020 | 3333 | 4374 | n/a | n/a |
| NZ_JROO01000009.1; | | | | | |
| 193; 194; 20/21 | | | | | |
| 98; 755108320; | 2046 | 3378 | 4399 | n/a | n/a |
| NZ_BBPN01000056.1; | | | | | |
| 195; 196; 16/17 | | | | | |
| 99; 755108320; | 2049 | 3380 | 4402 | n/a | n/a |
| NZ_BBPN01000056.1; | | | | | |
| 197; 198; 16/17 | | | | | |
| 100; 755077919; | 2047 | 3612 | 4400 | n/a | n/a |
| NZ_BBPQ01000048.1; | | | | | |
| 199; 200; 16/17 | | | | | |
| 101; 755077919; | 2048 | 3613 | 4401 | n/a | n/a |
| NZ_BBPQ01000048.1; | | | | | |
| 201; 202; 16/17 | | | | | |
| 102; 167643973; | 2136 | 2697 | n/a | n/a | n/a |
| NC_010338.1; | | | | | |
| 203; 204; 19/20 | | | | | |
| 103; 167643973; | 2136 | 2697 | n/a | n/a | n/a |
| NC_010338.1; | | | | | |
| 205; 206; 19/20 | | | | | |
| 104; 646523831; | 1607 | 2708 | n/a | n/a | n/a |
| NZ_BATN01000047.1; | | | | | |
| 207; 208; 18/19 | | | | | |
| 105; 646523831; | 2231 | 3420 | n/a | n/a | n/a |
| NZ_BATN01000047.1; | | | | | |
| 209; 210; 18/19 | | | | | |
| 106; 739598481; | 2190 | 3237 | n/a | n/a | n/a |
| NZ_JFHR01000062.1; | | | | | |
| 211; 212; 18/19 | | | | | |
| 107; 739598481; | 2190 | 3237 | n/a | n/a | n/a |
| NZ_JFHR01000062.1; | | | | | |
| 213; 214; 18/19 | | | | | |
| 108; 484272664; | 2203 | 3239 | n/a | n/a | n/a |
| NZ_AKIB01000015.1; | | | | | |
| 215; 216; 18/19 | | | | | |
| 109; 484272664; | 1666 | 2805 | n/a | n/a | n/a |
| NZ_AKIB01000015.1; | | | | | |
| 217; 218; 18/19 | | | | | |
| 110; 646523831; | 2241 | 2972 | n/a | n/a | n/a |
| NZ_BATN01000047.1; | | | | | |
| 219; 220; 18/19 | | | | | |
| 111; 312794749; | 2033 | 2722 | n/a | n/a | n/a |
| NC_014722.1; | | | | | |
| 221; 222; 10/11 | | | | | |
| 112; 312794749; | n/a | 2721 | n/a | n/a | n/a |
| NC_014722.1; | | | | | |
| 223; 224; 25/26 | | | | | |
| 113; 652527059; | n/a | 3434 | n/a | n/a | n/a |
| NZ_KE384226.1; | | | | | |
| 225; 226; 27/28 | | | | | |
| 114; 652527059; | n/a | 3007 | n/a | n/a | n/a |
| NZ_KE384226.1; | | | | | |
| 227; 228; 27/28 | | | | | |
| 115; 652527059; | 1790 | 3006 | n/a | n/a | n/a |
| NZ_KE384226.1; | | | | | |
| 229; 230; 28/29 | | | | | |
| 116; 652527059; | 1790 | 3006 | n/a | n/a | n/a |
| NZ_KE384226.1; | | | | | |
| 231; 232; 29/30 | | | | | |
| 117; 652527059; | 1790 | 3006 | n/a | n/a | n/a |
| NZ_KE384226.1; | | | | | |
| 233; 234; 28/29 | | | | | |
| 118; 483624586; | n/a | 2883 | n/a | n/a | n/a |
| NZ_KB889561.1; | | | | | |
| 235; 236; 23/24 | | | | | |
| 119; 221717172; | 1425 | 2481 | 3856 | n/a | n/a |
| DS999644.1; | | | | | |
| 237; 238; 27/28 | | | | | |
| 120; 221717172; | 1569 | 3148 | 3935 | n/a | n/a |
| DS999644.1; | | | | | |
| 239; 240; 27/28 | | | | | |
| 121; 221717172; | 1917 | 3526 | 3935 | n/a | n/a |
| DS999644.1; | | | | | |
| 241; 242; 27/28 | | | | | |
| 122; 221717172; | 1918 | 3536 | 3935 | n/a | n/a |
| DS999644.1; | | | | | |
| 243; 244; 27/28 | | | | | |
| 123; 664184565; | 1443 | 2505 | 3864 | n/a | n/a |
| NZ_JOGA01000019.1; | | | | | |
| 245; 246; 27/28 | | | | | |
| 124; 664184565; | 1919 | 3151 | 4305 | n/a | n/a |
| NZ_JOGA01000019.1; | | | | | |
| 247; 248; 27/28 | | | | | |
| 125; 764464761; | 1568 | 3140 | 3965 | n/a | n/a |
| NZ_JYBE01000113.1; | | | | | |
| 249; 250; 27/28 | | | | | |
| 126; 664184565; | 1882 | 3146 | 3965 | n/a | n/a |
| NZ_JOGA01000019.1; | | | | | |
| 251; 252; 27/28 | | | | | |
| 127; 764464761; | 1890 | 3156 | 3965 | n/a | n/a |
| NZ_JYBE01000113.1; | | | | | |
| 253; 254; 27/28 | | | | | |
| 128; 764464761; | 1452 | 2516 | 3867 | n/a | n/a |
| NZ_JYBE01000113.1; | | | | | |
| 255; 256; 27/28 | | | | | |
| 129; 764464761; | 1890 | 3411 | 3965 | n/a | n/a |
| NZ_JYBE01000113.1; | | | | | |
| 257; 258; 27/28 | | | | | |
| 130; 664051798; | 1873 | 3145 | 4269 | n/a | n/a |
| NZ_JNZK01000024.1; | | | | | |
| 259; 260; 27/28 | | | | | |
| 131; 664095100; | 1859 | 3154 | 4248 | n/a | n/a |
| NZ_JOED01000028.1; | | | | | |
| 261; 262; 24/25 | | | | | |
| 132; 664095100; | 1859 | 3147 | 4248 | n/a | n/a |
| NZ_JOED01000028.1; | | | | | |
| 263; 264; 24/25 | | | | | |
| 133; 664095100; | 1852 | 3531 | 4292 | n/a | n/a |
| NZ_JOED01000028.1; | | | | | |
| 265; 266; 24/25 | | | | | |
| 134; 664095100; | 1852 | 3123 | 4248 | n/a | n/a |
| NZ_JOED01000028.1; | | | | | |
| 267; 268; 24/25 | | | | | |
| 135; 664095100; | 1852 | 3649 | 4248 | n/a | n/a |
| NZ_JOED01000028.1; | | | | | |
| 269; 270; 24/25 | | | | | |
| 136; 664095100; | 1852 | 3144 | 4248 | n/a | n/a |
| NZ_JOED01000028.1; | | | | | |
| 271; 272; 24/25 | | | | | |
| 137; 664095100; | 1852 | 3141 | 4248 | n/a | n/a |
| NZ_JOED01000028.1; | | | | | |
| 273; 274; 24/25 | | | | | |
| 138; 664095100; | 1852 | 3534 | 4248 | n/a | n/a |
| NZ_JOED01000028.1; | | | | | |
| 275; 276; 24/25 | | | | | |
| 139; 664095100; | 1859 | 3530 | 4248 | n/a | n/a |
| NZ_JOED01000028.1; | | | | | |
| 277; 278; 24/25 | | | | | |
| 140; 664095100; | 1883 | 3527 | 4276 | n/a | n/a |
| NZ_JOED01000028.1; | | | | | |
| 279; 280; 24/25 | | | | | |
| 141; 664095100; | 1852 | 3391 | 4248 | n/a | n/a |
| NZ_JOED01000028.1; | | | | | |
| 281; 282; 24/25 | | | | | |
| 142; 664095100; | 1852 | 3528 | 4248 | n/a | n/a |
| NZ_JOED01000028.1; | | | | | |
| 283; 284; 24/25 | | | | | |
| 143; 484070161; | 1708 | 2862 | 4109 | n/a | n/a |
| NZ_KB898999.1; | | | | | |
| 285; 286; 24/25 | | | | | |
| 144; 664095100; | 1852 | 3529 | 4248 | n/a | n/a |
| NZ_JOED01000028.1; | | | | | |
| 287; 288; 24/25 | | | | | |
| 145; 664095100; | 1883 | 3651 | 4276 | n/a | n/a |
| NZ_JOED01000028.1; | | | | | |
| 289; 290; 24/25 | | | | | |
| 146; 664095100; | 1878 | 3152 | 4247 | n/a | n/a |
| NZ_JOED01000028.1; | | | | | |
| 291; 292; 24/25 | | | | | |
| 147; 664095100; | 1851 | 3153 | 4247 | n/a | n/a |
| NZ_JOED01000028.1; | | | | | |
| 293; 294; 24/25 | | | | | |
| 148; 664049400; | 1872 | 3176 | 4268 | n/a | n/a |
| NZ_JOEZ01000021.1; | | | | | |
| 295; 296; 24/25 | | | | | |
| 149; 695845602; | 1343 | 2375 | 3782 | n/a | n/a |
| NZ_JNWU01000018.1; | | | | | |
| 297; 298; 24/25 | | | | | |
| 150; 695845602; | 1645 | 3404 | 4413 | n/a | n/a |
| NZ_JNWU01000018.1; | | | | | |
| 299; 300; 24/25 | | | | | |
| 151; 695845602; | 1916 | 3143 | 4304 | n/a | n/a |
| NZ_JNWU01000018.1; | | | | | |
| 301; 302; 24/25 | | | | | |
| 152; 943927948; | 1902 | 3150 | 4296 | n/a | n/a |
| NZ_LIQV01000315.1; | | | | | |
| 303; 304; 24/25 | | | | | |
| 153; 654969845; | 2256 | 3647 | 4119 | n/a | n/a |
| NZ_ARPF01000020.1; | | | | | |
| 305; 306; 16/17 | | | | | |
| 154; 664095100; | 1869 | 3149 | 4265 | n/a | n/a |
| NZ_JOED01000028.1; | | | | | |
| 307; 308; 24/25 | | | | | |
| 155; 664021017; | 1869 | 3149 | 4265 | n/a | n/a |
| NZ_JOEM01000009.1; | | | | | |
| 309; 310; 26/27 | | | | | |
| 156; 664095100; | 1702 | 2856 | 4108 | n/a | n/a |
| NZ_JOED01000028.1; | | | | | |
| 311; 312; 24/25 | | | | | |
| 157; 654969845; | 1701 | 2855 | 4107 | n/a | n/a |
| NZ_ARPF01000020.1; | | | | | |
| 313; 314; 16/17 | | | | | |
| 158; 654969845; | 1821 | 3142 | 4119 | n/a | n/a |
| NZ_ARPF01000020.1; | | | | | |
| 315; 316; 16/17 | | | | | |
| 159; 221717172; | 1391 | 2441 | 3829 | n/a | n/a |
| DS999644.1; | | | | | |
| 317; 318; 27/28 | | | | | |
| 160; 315497051; | 1334 | 2360 | n/a | n/a | n/a |
| NC_014816.1; | | | | | |
| 319; 320; 28/29 | | | | | |
| 161; 315497051; | 1612 | 3364 | n/a | n/a | n/a |
| NC_014816.1; | | | | | |
| 321; 322; 28/29 | | | | | |
| 162; 380356103; | 1368 | 2406 | 3803 | n/a | n/a |
| AB593691.1; | | | | | |
| 323; 324; 26/27 | | | | | |
| 163; 383755859; | 1369 | 2407 | n/a | n/a | n/a |
| NC_017075.1; | | | | | |
| 325; 326; 20/21 | | | | | |
| 164; 383755859; | 1630 | 3401 | n/a | n/a | n/a |
| NC_017075.1; | | | | | |
| 327; 328; 20/21 | | | | | |
| 165; 381171950; | 2146 | 2596 | n/a | n/a | n/a |
| NZ_CAHO01000029.1; | | | | | |
| 329; 330; 29/30 | | | | | |
| 166; 325923334; | 1534 | 2622 | n/a | n/a | n/a |
| NZ_AEQX01000392.1; | | | | | |
| 331; 332; 26/27 | | | | | |
| 167; 325923334; | 1534 | 2622 | n/a | n/a | n/a |
| NZ_AEQX01000392.1; | | | | | |
| 333; 334; 28/29 | | | | | |
| 168; 565808720; | 2065 | 2946 | n/a | n/a | n/a |
| NZ_CM002307.1; | | | | | |
| 335; 336; 26/27 | | | | | |
| 169; 565808720; | 2065 | 2946 | n/a | n/a | n/a |
| NZ_CM002307.1; | | | | | |
| 337; 338; 28/29 | | | | | |
| 170; 825139250; | 2099 | 3467 | n/a | n/a | n/a |
| NZ_JZEH01000001.1; | | | | | |
| 339; 340; 26/27 | | | | | |
| 171; 325923334; | 2099 | 3467 | n/a | n/a | n/a |
| NZ_AEQX01000392.1; | | | | | |
| 341; 342; 28/29 | | | | | |
| 172; 507418017; | 2008 | 3314 | n/a | n/a | n/a |
| NZ_APMC02000050.1; | | | | | |
| 343; 344; 26/27 | | | | | |
| 173; 746486416; | 2008 | 3314 | n/a | n/a | n/a |
| NZ_KL638873.1; | | | | | |
| 345; 346; 28/29 | | | | | |
| 174; 746366822; | 2010 | 3316 | n/a | n/a | n/a |
| NZ_JSZF01000067.1; | | | | | |
| 347; 348; 26/27 | | | | | |
| 175; 746366822; | 2010 | 3316 | n/a | n/a | n/a |
| NZ_JSZF01000067.1; | | | | | |
| 349; 350; 28/29 | | | | | |
| 176; 825156557; | 2100 | 3468 | n/a | n/a | n/a |
| NZ_JZEI01000001.1; | | | | | |
| 351; 352; 25/26 | | | | | |
| 177; 920684790; | 2100 | 3468 | n/a | n/a | n/a |
| NZ_LHBW01000046.1; | | | | | |
| 353; 354; 28/29 | | | | | |
| 178; 507418017; | 2091 | 3451 | n/a | n/a | n/a |
| NZ_APMC02000050.1; | | | | | |
| 355; 356; 26/27 | | | | | |
| 179; 810489403; | 2091 | 3451 | n/a | n/a | n/a |
| NZ_CP011256.1; | | | | | |
| 357; 358; 28/29 | | | | | |
| 180; 746366822; | 2006 | 3312 | n/a | n/a | n/a |
| NZ_JSZF01000067.1; | | | | | |
| 359; 360; 26/27 | | | | | |
| 181; 746366822; | 2006 | 3312 | n/a | n/a | n/a |
| NZ_JSZF01000067.1; | | | | | |
| 361; 362; 28/29 | | | | | |
| 182; 507418017; | 2007 | 3313 | n/a | n/a | n/a |
| NZ_APMC02000050.1; | | | | | |
| 363; 364; 26/27 | | | | | |
| 183; 507418017; | 2007 | 3313 | n/a | n/a | n/a |
| NZ_APMC02000050.1; | | | | | |
| 365; 366; 28/29 | | | | | |
| 184; 507418017; | 1665 | 3323 | n/a | n/a | n/a |
| NZ_APMC02000050.1; | | | | | |
| 367; 368; 26/27 | | | | | |
| 185; 507418017; | 1665 | 3323 | n/a | n/a | n/a |
| NZ_APMC02000050.1; | | | | | |
| 369; 370; 28/29 | | | | | |
| 186; 507418017; | 2007 | 3386 | n/a | n/a | n/a |
| NZ_APMC02000050.1; | | | | | |
| 371; 372; 26/27 | | | | | |
| 187; 507418017; | 2007 | 3386 | n/a | n/a | n/a |
| NZ_APMC02000050.1; | | | | | |
| 373; 374; 28/29 | | | | | |
| 188; 746494072; | 2009 | 3315 | n/a | n/a | n/a |
| NZ_KL638866.1; | | | | | |
| 375; 376; 26/27 | | | | | |
| 189; 507418017; | 2009 | 3315 | n/a | n/a | n/a |
| NZ_APMC02000050.1; | | | | | |
| 377; 378; 28/29 | | | | | |
| 190; 507418017; | 1665 | 2804 | n/a | n/a | n/a |
| NZ_APMC02000050.1; | | | | | |
| 379; 380; 26/27 | | | | | |
| 191; 507418017; | 1665 | 2804 | n/a | n/a | n/a |
| NZ_APMC02000050.1; | | | | | |
| 381; 382; 28/29 | | | | | |
| 192; 507418017; | 2245 | 3633 | n/a | n/a | n/a |
| NZ_APMC02000050.1; | | | | | |
| 383; 384; 26/27 | | | | | |
| 193; 920684790; | 2245 | 3633 | n/a | n/a | n/a |
| NZ_LHBW01000046.1; | | | | | |
| 385; 386; 28/29 | | | | | |
| 194; 941965142; | 1477 | 2551 | n/a | n/a | n/a |
| NZ_LKIT01000002.1; | | | | | |
| 387; 388; 26/27 | | | | | |
| 195; 941965142; | 1477 | 2551 | n/a | n/a | n/a |
| NZ_LKIT01000002.1; | | | | | |
| 389; 390; 29/30 | | | | | |
| 196; 893711378; | 1574 | 2663 | n/a | n/a | n/a |
| NZ_KQ236029.1; | | | | | |
| 391; 392; 23/24 | | | | | |
| 197; 893711378; | 2125 | 3501 | n/a | n/a | n/a |
| NZ_KQ236029.1; | | | | | |
| 393; 394; 23/24 | | | | | |
| 198; 893711378; | 1676 | 2818 | n/a | n/a | n/a |
| NZ_KQ236029.1; | | | | | |
| 395; 396; 23/24 | | | | | |
| 199; 763092879; | 2066 | 3403 | n/a | n/a | n/a |
| NZ_JXZE01000003.1; | | | | | |
| 397; 398; 23/24 | | | | | |
| 200; 103485498; | 1320 | 2342 | n/a | n/a | n/a |
| NC_008048.1; | | | | | |
| 399; 400; 18/19 | | | | | |
| 201; 103485498; | 1320 | 2342 | n/a | n/a | n/a |
| NC_008048.1; | | | | | |
| 401; 402; 21/22 | | | | | |
| 202; 103485498; | 2134 | 3357 | n/a | n/a | n/a |
| NC_008048.1; | | | | | |
| 403; 404; 18/19 | | | | | |
| 203; 103485498; | 2134 | 3357 | n/a | n/a | n/a |
| NC_008048.1; | | | | | |
| 405; 406; 21/22 | | | | | |
| 204; 924898949; | 1361 | 2396 | n/a | n/a | n/a |
| NZ_CP009452.1; | | | | | |
| 407; 408; 21/22 | | | | | |
| 205; 738613868; | 1964 | 3217 | n/a | n/a | n/a |
| NZ_JFYZ01000002.1; | | | | | |
| 409; 410; 21/22 | | | | | |
| 206; 834156795; | n/a | 2497 | n/a | n/a | n/a |
| BBRO01000001.1; | | | | | |
| 411; 412; 12/13 | | | | | |
| 207; 834156795; | n/a | 2506 | n/a | n/a | n/a |
| BBRO01000001.1; | | | | | |
| 413; 414; 12/13 | | | | | |
| 208; 834156795; | 1985 | 3251 | n/a | n/a | n/a |
| BBRO01000001.1; | | | | | |
| 415; 416; 12/13 | | | | | |
| 209; 924898949; | 2255 | 3646 | n/a | n/a | n/a |
| NZ_CP009452.1; | | | | | |
| 417; 418; 21/22 | | | | | |
| 210; 937372567; | 2281 | 3689 | n/a | n/a | n/a |
| NZ_CP012700.1; | | | | | |
| 419; 420; 20/21 | | | | | |
| 211; 834156795; | 1434 | 2495 | n/a | n/a | n/a |
| BBRO01000001.1; | | | | | |
| 421; 422; 21/22 | | | | | |
| 212; 834156795; | 1434 | 2495 | n/a | n/a | n/a |
| BBRO01000001.1; | | | | | |
| 423; 424; 12/13 | | | | | |
| 213; 103485498; | 1321 | 2343 | n/a | n/a | n/a |
| NC_008048.1; | | | | | |
| 425; 426; 21/22 | | | | | |
| 214; 103485498; | 2028 | 3358 | n/a | n/a | n/a |
| NC_008048.1; | | | | | |
| 427; 428; 21/22 | | | | | |
| 215; 167621728; | 1597 | 2696 | n/a | n/a | n/a |
| NC_010335.1; | | | | | |
| 429; 430; 23/24 | | | | | |
| 216; 167621728; | 1597 | 2696 | n/a | n/a | n/a |
| NC_010335.1; | | | | | |
| 431; 432; 23/24 | | | | | |
| 217; 167621728; | 1597 | 2696 | n/a | n/a | n/a |
| NC_010335.1; | | | | | |
| 433; 434; 23/24 | | | | | |
| 218; 196476886; | 1326 | 2351 | n/a | n/a | n/a |
| CP000747.1; | | | | | |
| 435; 436; 16/17 | | | | | |
| 219; 295429362; | 1331 | 2356 | n/a | n/a | n/a |
| CP002008.1; | | | | | |
| 437; 438; 21/22 | | | | | |
| 220; 295429362; | 1331 | 2356 | n/a | n/a | n/a |
| CP002008.1; | | | | | |
| 439; 440; 18/19 | | | | | |
| 221; 295429362; | 1331 | 2356 | n/a | n/a | n/a |
| CP002008.1; | | | | | |
| 441; 442; 23/24 | | | | | |
| 222; 654573246; | 1817 | 3554 | n/a | n/a | n/a |
| NZ_AUEO01000025.1; | | | | | |
| 443; 444; 21/22 | | | | | |
| 223; 654573246; | 1817 | 3554 | n/a | n/a | n/a |
| NZ_AUEO01000025.1; | | | | | |
| 445; 446; 18/19 | | | | | |
| 224; 654573246; | 1817 | 3554 | n/a | n/a | n/a |
| NZ_AUEO01000025.1; | | | | | |
| 447; 448; 41/42 | | | | | |
| 225; 297196766; | 1389 | 2437 | 3825 | n/a | n/a |
| NZ_CM000951.1; | | | | | |
| 449; 450; 24/25 | | | | | |
| 226; 297196766; | n/a | 3543 | 3944 | n/a | n/a |
| NZ_CM000951.1; | | | | | |
| 451; 452; 24/25 | | | | | |
| 227; 754819815; | 1378 | 2424 | 3817 | n/a | n/a |
| NZ_CDME01000002.1; | | | | | |
| 453; 454; 24/25 | | | | | |
| 228; 754819815; | 1378 | 2424 | 3817 | n/a | n/a |
| NZ_CDME01000002.1; | | | | | |
| 455; 456; 24/25 | | | | | |
| 229; 754819815; | 2042 | 3615 | 4396 | n/a | n/a |
| NZ_CDME01000002.1; | | | | | |
| 457; 458; 24/25 | | | | | |
| 230; 754819815; | 2042 | 3615 | 4396 | n/a | n/a |
| NZ_CDME01000002.1; | | | | | |
| 459; 460; 24/25 | | | | | |
| 231; 487385965; | 1719 | 2878 | 4123 | n/a | n/a |
| NZ_KB911613.1; | | | | | |
| 461; 462; 23/24 | | | | | |
| 232; 487385965; | 1719 | 2878 | 4123 | n/a | n/a |
| NZ_KB911613.1; | | | | | |
| 463; 464; 22/23 | | | | | |
| 233; 458977979; | 1403 | 2457 | 3837 | n/a | n/a |
| NZ_AORZ01000024.1; | | | | | |
| 465; 466; 16/17 | | | | | |
| 234; 458977979; | 1528 | 3549 | 3930 | n/a | n/a |
| NZ_AORZ01000024.1; | | | | | |
| 467; 468; 16/17 | | | | | |
| 235; 825314728; | 2239 | 3470 | n/a | n/a | n/a |
| NZ_LASZ01000003.1; | | | | | |
| 469; 470; 26/27 | | | | | |
| 236; 483972948; | 1704 | 2858 | 4185 | n/a | n/a |
| NZ_KB891808.1; | | | | | |
| 471; 472; 28/29 | | | | | |
| 237; 937505789; | 1476 | 2550 | n/a | n/a | n/a |
| NZ_LJGM01000026.1; | | | | | |
| 473; 474; 26/27 | | | | | |
| 238; 938883590; | 2283 | 3692 | n/a | n/a | n/a |
| NZ_CP012900.1; | | | | | |
| 475; 476; 25/26 | | | | | |
| 239; 663737675; | 2191 | 3572 | 4263 | n/a | n/a |
| NZ_JOJF01000002.1; | | | | | |
| 477; 478; 29/30 | | | | | |
| 240; 835885587; | 2104 | 3593 | n/a | n/a | n/a |
| NZ_KN265462.1; | | | | | |
| 479; 480; 26/27 | | | | | |
| 241; 825314716; | 2101 | 3469 | n/a | n/a | n/a |
| NZ_LASZ01000002.1; | | | | | |
| 481; 482; 26/27 | | | | | |
| 242; 67639376; | 1449 | 2512 | n/a | n/a | n/a |
| NZ_AAHO01000116.1; | | | | | |
| 483; 484; 28/29 | | | | | |
| 243; 835885587; | 1448 | 2510 | n/a | n/a | n/a |
| NZ_KN265462.1; | | | | | |
| 485; 486; 33/34 | | | | | |
| 244; 433601838; | n/a | 2758 | 4044 | n/a | n/a |
| NC_019673.1; | | | | | |
| 487; 488; 26/27 | | | | | |
| 245; 653330442; | 1812 | 3032 | n/a | n/a | n/a |
| NZ_KE386531.1; | | | | | |
| 489; 490; 26/27 | | | | | |
| 246; 389798210; | 1543 | 2633 | n/a | n/a | n/a |
| NZ_AJXV01000032.1; | | | | | |
| 491; 492; 26/27 | | | | | |
| 247; 469816339; | 1643 | 2769 | n/a | n/a | n/a |
| NC_020541.1; | | | | | |
| 493; 494; 26/27 | | | | | |
| 248; 653308965; | 1809 | 3029 | n/a | n/a | n/a |
| NZ_AXBJ01000026.1; | | | | | |
| 495; 496; 24/25 | | | | | |
| 249; 919546651; | n/a | 3629 | n/a | n/a | n/a |
| NZ_JOEL01000060.1; | | | | | |
| 497; 498; 27/28 | | | | | |
| 250; 653321547; | 1810 | 3030 | n/a | n/a | n/a |
| NZ_ATYF01000013.1; | | | | | |
| 499; 500; 26/27 | | | | | |
| 251; 332527785; | 1564 | 2658 | n/a | n/a | n/a |
| NZ_AEWG01000155.1; | | | | | |
| 501; 502; 20/21 | | | | | |
| 252; 269954810; | 1605 | 3541 | 4000 | n/a | n/a |
| NC_013530.1; | | | | | |
| 503; 504; 20/21 | | | | | |
| 253; 943674269; | 1656 | 3565 | 4070 | n/a | n/a |
| NZ_LIQO01000205.1; | | | | | |
| 505; 506; 21/22 | | | | | |
| 254; 663414324; | 1656 | 2794 | 4070 | n/a | n/a |
| NZ_JOHQ01000068.1; | | | | | |
| 507; 508; 21/22 | | | | | |
| 255; 943674269; | 1656 | 3568 | 4070 | n/a | n/a |
| NZ_LIQO01000205.1; | | | | | |
| 509; 510; 21/22 | | | | | |
| 256; 269954810; | 1328 | 2353 | 3765 | n/a | n/a |
| NC_013530.1; | | | | | |
| 511; 512; 20/21 | | | | | |
| 257; 937505789; | 1760 | 3516 | n/a | n/a | n/a |
| NZ_LJGM01000026.1; | | | | | |
| 513; 514; 26/27 | | | | | |
| 258; 663414324; | 1864 | 3563 | 4070 | n/a | n/a |
| NZ_JOHQ01000068.1; | | | | | |
| 515; 516; 21/22 | | | | | |
| 259; 663414324; | 1656 | 3575 | 4070 | n/a | n/a |
| NZ_JOHQ01000068.1; | | | | | |
| 517; 518; 21/22 | | | | | |
| 260; 389759651; | 1548 | 3229 | n/a | n/a | n/a |
| NZ_AJXS01000437.1; | | | | | |
| 519; 520; 26/27 | | | | | |
| 261; 928998800; | 2274 | 3675 | n/a | n/a | n/a |
| NZ_BBYR01000083.1; | | | | | |
| 521; 522; 16/17 | | | | | |
| 262; 943674269; | 1656 | 3673 | 4070 | n/a | n/a |
| NZ_LIQO01000205.1; | | | | | |
| 523; 524; 21/22 | | | | | |
| 263; 856992287; | 2113 | 3484 | 4458 | n/a | n/a |
| NZ_LFKW01000127.1; | | | | | |
| 525; 526; 20/21 | | | | | |
| 264; 938956730; | 2285 | 3694 | n/a | n/a | n/a |
| NZ_CP009429.1; | | | | | |
| 527; 528; 19/20 | | | | | |
| 265; 563282524; | 1419 | 2474 | n/a | n/a | n/a |
| AYSC01000019.1; | | | | | |
| 529; 530; 22/23 | | | | | |
| 266; 399058618; | 1545 | 2636 | n/a | n/a | n/a |
| NZ_AKKE01000021.1; | | | | | |
| 531; 532; 22/23 | | | | | |
| 267; 937372567; | n/a | 3690 | n/a | n/a | n/a |
| NZ_CP012700.1; | | | | | |
| 533; 534; 19/20 | | | | | |
| 268; 825353621; | 2102 | 3471 | 4445 | n/a | n/a |
| NZ_LAYX01000011.1; | | | | | |
| 535; 536; 21/22 | | | | | |
| 269; 937505789; | 2282 | 3691 | n/a | n/a | n/a |
| NZ_LJGM01000026.1; | | | | | |
| 537; 538; 26/27 | | | | | |
| 270; 739702045; | 1446 | 2508 | n/a | n/a | n/a |
| NZ_JNFC01000030.1; | | | | | |
| 539; 540; 18/19 | | | | | |
| 271; 484867900; | n/a | 3448 | 4110 | n/a | n/a |
| NZ_AGNH01000612.1; | | | | | |
| 541; 542; 15/16 | | | | | |
| 272; 162960844; | 1989 | 3257 | 4349 | n/a | n/a |
| NC_003155.4; | | | | | |
| 543; 544; 23/24 | | | | | |
| 273; 162960844; | n/a | 2403 | 3800 | n/a | n/a |
| NC_003155.4; | | | | | |
| 545; 546; 23/24 | | | | | |
| 274; 399069941; | 1544 | 2635 | n/a | n/a | n/a |
| NZ_AKKF01000033.1; | | | | | |
| 547; 548; 22/23 | | | | | |
| 275; 399069941; | 1544 | 2635 | n/a | n/a | n/a |
| NZ_AKKF01000033.1; | | | | | |
| 549; 550; 22/23 | | | | | |
| 276; 738615271; | 1428 | 2485 | n/a | n/a | n/a |
| NZ_JFYZ01000008.1; | | | | | |
| 551; 552; 22/23 | | | | | |
| 277; 739659070; | 1445 | 2507 | n/a | n/a | n/a |
| NZ_JNFD01000017.1; | | | | | |
| 553; 554; 19/20 | | | | | |
| 278; 749188513; | 2011 | 3317 | n/a | n/a | n/a |
| NZ_CP009122.1; | | | | | |
| 555; 556; 19/20 | | | | | |
| 279; 345007964; | 1624 | 3548 | 4025 | n/a | n/a |
| NC_015957.1; | | | | | |
| 557; 558; 24/25 | | | | | |
| 280; 345007964; | 1624 | 3548 | 4025 | n/a | n/a |
| NC_015957.1; | | | | | |
| 559; 560; 24/25 | | | | | |
| 281; 345007964; | 1337 | 2364 | 3771 | n/a | n/a |
| NC_015957.1; | | | | | |
| 561; 562; 24/25 | | | | | |
| 282; 345007964; | 1337 | 2364 | 3771 | n/a | n/a |
| NC_015957.1; | | | | | |
| 563; 564; 24/25 | | | | | |
| 283; 928998724; | 1436 | 2498 | n/a | n/a | n/a |
| NZ_BBYR01000007.1; | | | | | |
| 565; 566; 19/20 | | | | | |
| 284; 484007841; | n/a | 2822 | 4087 | n/a | n/a |
| NZ_ANAD01000138.1; | | | | | |
| 567; 568; 20/21 | | | | | |
| 285; 162960844; | 1583 | 3256 | 4348 | n/a | n/a |
| NC_003155.4; | | | | | |
| 569; 570; 21/22 | | | | | |
| 286; 162960844; | 1366 | 2404 | 3801 | n/a | n/a |
| NC_003155.4; | | | | | |
| 571; 572; 21/22 | | | | | |
| 287; 662133033; | 1894 | 3271 | 4287 | n/a | n/a |
| NZ_KL570321.1; | | | | | |
| 573; 574; 21/22 | | | | | |
| 288; 662133033; | 1850 | 3494 | 4246 | n/a | n/a |
| NZ_KL570321.1; | | | | | |
| 575; 576; 21/22 | | | | | |
| 289; 487404592; | 1725 | 2886 | 4131 | n/a | n/a |
| NZ_ARVW01000001.1; | | | | | |
| 577; 578; 22/23 | | | | | |
| 290; 739659070; | 2215 | 3245 | n/a | n/a | n/a |
| NZ_JNFD01000017.1; | | | | | |
| 579; 580; 19/20 | | | | | |
| 291; 702808005; | 1925 | 3167 | 4311 | n/a | n/a |
| NZ_JNZA01000041.1; | | | | | |
| 581; 582; 21/22 | | | | | |
| 292; 664277815; | 1889 | 3574 | 4281 | n/a | n/a |
| NZ_JOIX01000041.1; | | | | | |
| 583; 584; 21/22 | | | | | |
| 293; 499136900; | 1972 | 3234 | 4345 | n/a | n/a |
| NZ_ASJB01000015.1; | | | | | |
| 585; 586; 20/21 | | | | | |
| 294; 487404592; | 1725 | 2886 | 4131 | n/a | n/a |
| NZ_ARVW01000001.1; | | | | | |
| 587; 588; 22/23 | | | | | |
| 295; 716912366; | 1928 | 3172 | 4314 | n/a | n/a |
| NZ_JRHJ01000016.1; | | | | | |
| 589; 590; 21/22 | | | | | |
| 296; 381200190; | 1567 | 2660 | 3964 | n/a | n/a |
| NZ_JH164855.1; | | | | | |
| 591; 592; 19/20 | | | | | |
| 297; 663300513; | 1856 | 3255 | 4252 | n/a | n/a |
| NZ_JNZY01000033.1; | | | | | |
| 593; 594; 21/22 | | | | | |
| 298; 822214995; | 1355 | 2388 | 3792 | n/a | n/a |
| NZ_CP007699.1; | | | | | |
| 595; 596; 21/22 | | | | | |
| 299; 664013282; | 1868 | 3261 | 4264 | n/a | n/a |
| NZ_JOAP01000011.1; | | | | | |
| 597; 598; 12/13 | | | | | |
| 300; 822214995; | 2095 | 3460 | 4441 | n/a | n/a |
| NZ_CP007699.1; | | | | | |
| 599; 600; 21/22 | | | | | |
| 301; 514916021; | 1409 | 2463 | 3841 | n/a | n/a |
| NZ_AOPZ01000017.1; | | | | | |
| 601; 602; 21/22 | | | | | |
| 302; 514916021; | 1658 | 3258 | 4071 | n/a | n/a |
| NZ_AOPZ01000017.1; | | | | | |
| 603; 604; 21/22 | | | | | |
| 303; 663421576; | 1865 | 3579 | 4260 | n/a | n/a |
| NZ_JOGE01000134.1; | | | | | |
| 605; 606; 21/22 | | | | | |
| 304; 928897596; | 2272 | 3672 | 4538 | n/a | n/a |
| NZ_LGKG01000207.1; | | | | | |
| 607; 608; 21/22 | | | | | |
| 305; 484007121; | n/a | 2756 | 4042 | n/a | n/a |
| NZ_ANAC01000010.1; | | | | | |
| 609; 610; 29/30 | | | | | |
| 306; 484007121; | 1779 | 3377 | 4042 | n/a | n/a |
| NZ_ANAC01000010.1; | | | | | |
| 611; 612; 29/30 | | | | | |
| 307; 646523831; | 2241 | 2972 | n/a | n/a | n/a |
| NZ_BATN001000047.1; | | | | | |
| 613; 614; 18/19 | | | | | |
| 308; 484007121; | 1779 | 2820 | 4042 | n/a | n/a |
| NZ_ANAC01000010.1; | | | | | |
| 615; 616; 29/30 | | | | | |
| 309; 651281457; | 1782 | 3556 | 4488 | n/a | n/a |
| NZ_JADG01000010.1; | | | | | |
| 617; 618; 19/20 | | | | | |
| 310; 664428976; | 1854 | 3080 | 4250 | n/a | n/a |
| NZ_KL585179.1; | | | | | |
| 619; 620; 21/22 | | | | | |
| 311; 926412104; | 2266 | 3663 | 4533 | n/a | n/a |
| NZ_LGDY01000113.1; | | | | | |
| 621; 622; 18/19 | | | | | |
| 312; 703210604; | n/a | 3169 | n/a | n/a | n/a |
| NZ_JNYM01000124.1; | | | | | |
| 623; 624; 44/45 | | | | | |
| 313; 471319476; | 1647 | 2774 | 4059 | n/a | n/a |
| NC_020504.1; | | | | | |
| 625; 626; 21/22 | | | | | |
| 314; 485454803; | 2057 | 3525 | 4408 | n/a | n/a |
| NZ_AFRP01001656.1; | | | | | |
| 627; 628; 21/22 | | | | | |
| 315; 664487325; | 1896 | 3157 | 4290 | n/a | n/a |
| NZ_JOJI01000036.1; | | | | | |
| 629; 630; 29/30 | | | | | |
| 316; 297189896; | 1390 | 2438 | 3826 | n/a | n/a |
| NZ_CM000950.1; | | | | | |
| 631; 632; 21/22 | | | | | |
| 317; 297189896; | 1531 | 3268 | 3933 | n/a | n/a |
| NZ_CM000950.1; | | | | | |
| 633; 634; 21/22 | | | | | |
| 318; 398790069; | 2040 | 3371 | 4394 | n/a | n/a |
| NZ_JH725387.1; | | | | | |
| 635; 636; 21/22 | | | | | |
| 319; 754221033; | n/a | 3277 | 4362 | n/a | n/a |
| NZ_CP007574.1; | | | | | |
| 637; 638; 22/23 | | | | | |
| 320; 928998724; | 2273 | 3674 | n/a | n/a | n/a |
| NZ_BBYR01000007.1; | | | | | |
| 639; 640; 19/20 | | | | | |
| 321; 931609467; | n/a | 3683 | 4543 | n/a | n/a |
| NZ_CP012752.1; | | | | | |
| 641; 642; 24/25 | | | | | |
| 322; 484017897; | 1776 | 2829 | 4124 | n/a | n/a |
| NZ_ANBB01000025.1; | | | | | |
| 643; 644; 20/21 | | | | | |
| 323; 943388237; | 2055 | 3606 | 4406 | n/a | n/a |
| NZ_LIQD01000001.1; | | | | | |
| 645; 646; 21/22 | | | | | |
| 324; 398790069; | 1536 | 2625 | 3938 | n/a | n/a |
| NZ_JH725387.1; | | | | | |
| 647; 648; 21/22 | | | | | |
| 325; 224581107; | 1517 | 2602 | 3926 | n/a | n/a |
| NZ_GG657757.1; | | | | | |
| 649; 650; 19/20 | | | | | |
| 326; 664245663; | 1888 | 3109 | 4279 | n/a | n/a |
| NZ_JODF01000003.1; | | | | | |
| 651; 652; 21/22 | | | | | |
| 327; 664026629; | 1870 | 3096 | 4266 | n/a | n/a |
| NZ_JOAP01000049.1; | | | | | |
| 653; 654; 21/22 | | | | | |
| 328; 764439507; | 1848 | 3410 | 4245 | n/a | n/a |
| NZ_JRKI01000027.1; | | | | | |
| 655; 656; 21/22 | | | | | |
| 329; 662059070; | 1845 | 3076 | 4242 | n/a | n/a |
| NZ_KL571162.1; | | | | | |
| 657; 658; 29/30 | | | | | |
| 330; 739830264; | 1991 | 3260 | 4352 | n/a | n/a |
| NZ_JOJE01000040.1; | | | | | |
| 659; 660; 21/22 | | | | | |
| 331; 662063073; | 2082 | 3432 | 4426 | n/a | n/a |
| NZ_JNXV01000303.1; | | | | | |
| 661; 662; 22/23 | | | | | |
| 332; 664141810; | 1881 | 3105 | 4275 | n/a | n/a |
| NZ_JOCQ01000106.1; | | | | | |
| 663; 664; 29/30 | | | | | |
| 333; 799161588; | n/a | 2525 | 3873 | n/a | n/a |
| NZ_JZWZ01000076.1; | | | | | |
| 665; 666; 25/26 | | | | | |
| 334; 664523889; | 1897 | 3603 | 4291 | n/a | n/a |
| NZ_JOFH01000020.1; | | | | | |
| 667; 668; 23/24 | | | | | |
| 335; 754862786; | 1767 | 2968 | 4177 | n/a | n/a |
| NZ_CP007155.1; | | | | | |
| 669; 670; 40/41 | | | | | |
| 336; 655416831; | 1828 | 3054 | 4226 | n/a | n/a |
| NZ_KE386846.1; | | | | | |
| 671; 672; 20/21 | | | | | |
| 337; 662063073; | n/a | 3077 | 4243 | n/a | n/a |
| NZ_JNXV01000303.1; | | | | | |
| 673; 674; 22/23 | | | | | |
| 338; 664523889; | 1993 | 3552 | 4354 | n/a | n/a |
| NZ_JOFH01000020.1; | | | | | |
| 675; 676; 23/24 | | | | | |
| 339; 663122276; | 1853 | 3252 | 4249 | n/a | n/a |
| NZ_JOFJ01000001.1; | | | | | |
| 677; 678; 20/21 | | | | | |
| 340; 654239557; | 1814 | 3269 | 4213 | n/a | n/a |
| NZ_AZWL01000018.1; | | | | | |
| 679; 680; 21/22 | | | | | |
| 341; 926344107; | 2260 | 3654 | 4525 | n/a | n/a |
| NZ_LGEA01000058.1; | | | | | |
| 681; 682; 19/20 | | | | | |
| 342; 765016627; | 2074 | 3416 | 4416 | n/a | n/a |
| NZ_LK022849.1; | | | | | |
| 683; 684; 22/23 | | | | | |
| 343; 765016627; | 2074 | 3416 | 4416 | n/a | n/a |
| NZ_LK022849.1; | | | | | |
| 685; 686; 22/23 | | | | | |
| 344; 755908329; | 1353 | 2385 | 3790 | n/a | n/a |
| CP007219.1; | | | | | |
| 687; 688; 20/21 | | | | | |
| 345; 664061406; | 1863 | 3668 | 3923 | n/a | n/a |
| NZ_JOES01000059.1; | | | | | |
| 689; 690; 29/30 | | | | | |
| 346; 799161588; | n/a | 3620 | 4431 | n/a | n/a |
| NZ_JZWZ01000076.1; | | | | | |
| 691; 692; 25/26 | | | | | |
| 347; 664061406; | 1514 | 3103 | 3923 | n/a | n/a |
| NZ_JOES01000059.1; | | | | | |
| 693; 694; 29/30 | | | | | |
| 348; 664434000; | 1516 | 2601 | 3925 | n/a | n/a |
| NZ_JOIA01001078.1; | | | | | |
| 695; 696; 21/22 | | | | | |
| 349; 429195484; | 2120 | 2653 | 3959 | n/a | n/a |
| NZ_AEJC01000118.1; | | | | | |
| 697; 698; 22/23 | | | | | |
| 350; 664325162; | 1892 | 3112 | 4284 | n/a | n/a |
| NZ_JOJB01000032.1; | | | | | |
| 699; 700; 21/22 | | | | | |
| 351; 664061406; | 1875 | 3160 | 3923 | n/a | n/a |
| NZ_JOES01000059.1; | | | | | |
| 701; 702; 29/30 | | | | | |
| 352; 657301257; | 2070 | 3412 | 4236 | n/a | n/a |
| NZ_AZSD01000480.1; | | | | | |
| 703; 704; 21/22 | | | | | |
| 353; 657301257; | n/a | 3486 | 4236 | n/a | n/a |
| NZ_AZSD01000480.1; | | | | | |
| 705; 706; 21/22 | | | | | |
| 354; 458984960; | 1529 | 3550 | 3931 | n/a | n/a |
| NZ_AORZ01000079.1; | | | | | |
| 707; 708; 12/13 | | | | | |
| 355; 657301257; | 1835 | 3066 | 4236 | n/a | n/a |
| NZ_AZSD01000480.1; | | | | | |
| 709; 710; 21/22 | | | | | |
| 356; 925315417; | 1863 | 3090 | 3923 | n/a | n/a |
| LGCQ01000244.1; | | | | | |
| 711; 712; 29/30 | | | | | |
| 357; 926371517; | 2262 | 3656 | 4527 | n/a | n/a |
| NZ_LGCW01000271.1; | | | | | |
| 713; 714; 29/30 | | | | | |
| 358; 925315417; | 1514 | 3101 | 3923 | n/a | n/a |
| LGCQ01000244.1; | | | | | |
| 715; 716; 29/30 | | | | | |
| 359; 664325162; | 1858 | 3084 | 4254 | n/a | n/a |
| NZ_JOJB01000032.1; | | | | | |
| 717; 718; 21/22 | | | | | |
| 360; 664061406; | 1514 | 3162 | 3923 | n/a | n/a |
| NZ_JOES01000059.1; | | | | | |
| 719; 720; 29/30 | | | | | |
| 361; 926403453; | 2265 | 3661 | 4530 | n/a | n/a |
| NZ_LGDD01000321.1; | | | | | |
| 721; 722; 21/22 | | | | | |
| 362; 671472153; | 1905 | 2915 | 4152 | n/a | n/a |
| NZ_JOFR01000001.1; | | | | | |
| 723; 724; 21/22 | | | | | |
| 363; 471319476; | 1646 | 2773 | 4058 | n/a | n/a |
| NC_020504.1; | | | | | |
| 725; 726; 18/19 | | | | | |
| 364; 739854483; | 1992 | 3262 | 4353 | n/a | n/a |
| NZ_KL997447.1; | | | | | |
| 727; 728; 21/22 | | | | | |
| 365; 926371520; | n/a | 2540 | 3884 | n/a | n/a |
| NZ_LGCW01000274.1; | | | | | |
| 729; 730; 27/28 | | | | | |
| 366; 485454803; | n/a | 3546 | n/a | n/a | n/a |
| NZ_AFRP01001656.1; | | | | | |
| 731; 732; 21/22 | | | | | |
| 367; 738615271; | 2182 | 3218 | n/a | n/a | n/a |
| NZ_JFYZ01000008.1; | | | | | |
| 733; 734; 21/22 | | | | | |
| 368; 738615271; | 2182 | 3218 | n/a | n/a | n/a |
| NZ_JFYZ01000008.1; | | | | | |
| 735; 736; 21/22 | | | | | |
| 369; 738615271; | 2182 | 3218 | n/a | n/a | n/a |
| NZ_JFYZ01000008.1; | | | | | |
| 737; 738; 22/23 | | | | | |
| 370; 664479796; | n/a | 3120 | n/a | n/a | n/a |
| NZ_JOJI01000005.1; | | | | | |
| 739; 740; 19/20 | | | | | |
| 371; 357397620; | 1628 | 2747 | 4035 | n/a | n/a |
| NC_016111.1; | | | | | |
| 741; 742; 13/14 | | | | | |
| 372; 665604093; | 1904 | 3126 | 4299 | n/a | n/a |
| NZ_JNXR01000023.1; | | | | | |
| 743; 744; 21/22 | | | | | |
| 373; 739674258; | 1981 | 3247 | n/a | n/a | n/a |
| NZ_JQMC01000050.1; | | | | | |
| 745; 746; 23/24 | | | | | |
| 374; 664061406; | 1461 | 2532 | 3876 | n/a | n/a |
| NZ_JOES01000059.1; | | | | | |
| 747; 748; 29/30 | | | | | |
| 375; 664061406; | 1467 | 2538 | 3882 | n/a | n/a |
| NZ_JOES01000059.1; | | | | | |
| 749; 750; 29/30 | | | | | |
| 376; 926371517; | 1469 | 2541 | 3885 | n/a | n/a |
| NZ_LGCW01000271.1; | | | | | |
| 751; 752; 29/30 | | | | | |
| 377; 664244706; | 1886 | 3108 | 4277 | n/a | n/a |
| NZ_JOBD01000002.1; | | | | | |
| 753; 754; 24/25 | | | | | |
| 378; 925315417; | 1463 | 2534 | 3878 | n/a | n/a |
| LGCQ01000244.1; | | | | | |
| 755; 756; 29/30 | | | | | |
| 379; 646529442; | 1769 | 2973 | n/a | n/a | n/a |
| NZ_BATN001000092.1; | | | | | |
| 757; 758; 18/19 | | | | | |
| 380; 906344334; | 2132 | 3513 | n/a | n/a | n/a |
| NZ_LFXA01000002.1; | | | | | |
| 759; 760; 12/13 | | | | | |
| 381; 926344331; | 2261 | 3655 | 4526 | n/a | n/a |
| NZ_LGEA01000105.1; | | | | | |
| 761; 762; 21/22 | | | | | |
| 382; 664421883; | 1893 | 3115 | 4286 | n/a | n/a |
| NZ_JODC01000023.1; | | | | | |
| 763; 764; 21/22 | | | | | |
| 383; 755134941; | 2240 | 3626 | n/a | n/a | n/a |
| NZ_BBPI01000030.1; | | | | | |
| 765; 766; 22/23 | | | | | |
| 384; 663596322; | 1866 | 3602 | 4261 | n/a | n/a |
| NZ_JOEF01000022.1; | | | | | |
| 767; 768; 21/22 | | | | | |
| 385; 664063830; | 1876 | 3098 | 4271 | n/a | n/a |
| NZ_JODT01000002.1; | | | | | |
| 769; 770; 13/14 | | | | | |
| 386; 484203522; | 1691 | 2842 | 4100 | n/a | n/a |
| NZ_AQUI01000002.1; | | | | | |
| 771; 772; 12/13 | | | | | |
| 387; 365867746; | 1394 | 2445 | 3832 | n/a | n/a |
| NZ_AGSW01000272.1; | | | | | |
| 773; 774; 22/23 | | | | | |
| 388; 759802587; | 2059 | 3399 | 4409 | n/a | n/a |
| NZ_CP009438.1; | | | | | |
| 775; 776; 21/22 | | | | | |
| 389; 664325162; | 1358 | 2393 | 3795 | n/a | n/a |
| NZ_JOJB01000032.1; | | | | | |
| 777; 778; 21/22 | | | | | |
| 390; 484008051; | 1680 | 2824 | 4089 | n/a | n/a |
| NZ_ANAD01000197.1; | | | | | |
| 779; 780; 24/25 | | | | | |
| 391; 458848256; | 1540 | 3327 | 3942 | n/a | n/a |
| NZ_AOH001000055.1; | | | | | |
| 781; 782; 21/22 | | | | | |
| 392; 458848256; | 1402 | 2456 | 3836 | n/a | n/a |
| NZ_AOHO01000055.1; | | | | | |
| 783; 784; 21/22 | | | | | |
| 393; 664478668; | 1855 | 3272 | 4251 | n/a | n/a |
| NZ_JOJI01000002.1; | | | | | |
| 785; 786; 19/20 | | | | | |
| 394; 484008051; | 1778 | 2825 | 4090 | n/a | n/a |
| NZ_ANAD01000197.1; | | | | | |
| 787; 788; 24/25 | | | | | |
| 395; 365867746; | n/a | 3155 | 3946 | n/a | n/a |
| NZ_AGSW01000272.1; | | | | | |
| 789; 790; 22/23 | | | | | |
| 396; 873282818; | n/a | 3487 | 4461 | n/a | n/a |
| NZ_LFEH01000123.1; | | | | | |
| 791; 792; 25/26 | | | | | |
| 397; 664061406; | 1514 | 3382 | 3923 | n/a | n/a |
| NZ_JOES01000059.1; | | | | | |
| 793; 794; 29/30 | | | | | |
| 398; 873282818; | n/a | 3466 | 4234 | n/a | n/a |
| NZ_LFEH01000123.1; | | | | | |
| 795; 796; 25/26 | | | | | |
| 399; 906344339; | 2133 | 3514 | 4471 | n/a | n/a |
| NZ_LFXA01000007.1; | | | | | |
| 797; 798; 19/20 | | | | | |
| 400; 759944049; | 2061 | 3609 | n/a | n/a | n/a |
| NZ_JOAG01000029.1; | | | | | |
| 799; 800; 28/29 | | | | | |
| 401; 557839714; | 1745 | 2913 | n/a | n/a | n/a |
| NZ_AWGF01000010.1; | | | | | |
| 801; 802; 28/29 | | | | | |
| 402; 695870063; | n/a | 3537 | 4306 | n/a | n/a |
| NZ_JNWW01000028.1; | | | | | |
| 803; 804; 23/24 | | | | | |
| 403; 749181963; | 2013 | 3598 | 4368 | n/a | n/a |
| NZ_CP003987.1; | | | | | |
| 805; 806; 12/13 | | | | | |
| 404; 852460626; | 1359 | 2394 | 3796 | n/a | n/a |
| CP011799.1; | | | | | |
| 807; 808; 13/14 | | | | | |
| 405; 374982757; | 1332 | 2357 | 3767 | n/a | 3768 |
| NC_016582.1; | | | | | |
| 809; 810; 13/14 | | | | | |
| 406; 374982757; | 1332 | 2357 | 3767 | n/a | 3768 |
| NC_016582.1; | | | | | |
| 811; 812; 28/29 | | | | | |
| 407; 914607448; | n/a | 2529 | n/a | n/a | n/a |
| NZ_JYNE01000028.1; | | | | | |
| 813; 814; 22/23 | | | | | |
| 408; 663373497; | 1861 | 3088 | 4257 | n/a | n/a |
| NZ_JOFL01000043.1; | | | | | |
| 815; 816; 19/20 | | | | | |
| 409; 764442321; | n/a | 3625 | 4415 | n/a | n/a |
| NZ_JRKI01000041.1; | | | | | |
| 817; 818; 29/30 | | | | | |
| 410; 739702045; | 2214 | 3250 | n/a | n/a | n/a |
| NZ_JNFC01000030.1; | | | | | |
| 819; 820; 18/19 | | | | | |
| 411; 485090585; | n/a | 2870 | 4115 | n/a | n/a |
| NZ_KB907209.1; | | | | | |
| 821; 822; 20/21 | | | | | |
| 412; 764442321; | 1847 | 3586 | 4501 | n/a | n/a |
| NZ_JRKI01000041.1; | | | | | |
| 823; 824; 29/30 | | | | | |
| 413; 514916412; | 1659 | 3591 | 4350 | n/a | n/a |
| NZ_AOPZ01000028.1; | | | | | |
| 825; 826; 33/34 | | | | | |
| 414; 514916412; | 1408 | 2462 | 3840 | n/a | n/a |
| NZ_AOPZ01000028.1; | | | | | |
| 827; 828; 33/34 | | | | | |
| 415; 970574347; | 1839 | 2873 | 4118 | n/a | n/a |
| NZ_LNZF01000001.1; | | | | | |
| 829; 830; 20/21 | | | | | |
| 416; 970574347; | 1768 | 2969 | 4084 | n/a | n/a |
| NZ_LNZF01000001.1; | | | | | |
| 831; 832; 20/21 | | | | | |
| 417; 906292938; | 1915 | 3139 | n/a | n/a | n/a |
| CXPB01000073.1; | | | | | |
| 833; 834; 18/19 | | | | | |
| 418; 906292938; | 1383 | 2431 | n/a | n/a | n/a |
| CXPB01000073.1; | | | | | |
| 835; 836; 18/19 | | | | | |
| 419; 970574347; | 1662 | 2799 | 4074 | n/a | n/a |
| NZ_LNZF01000001.1; | | | | | |
| 837; 838; 20/21 | | | | | |
| 420; 671525382; | n/a | 3130 | 4496 | n/a | n/a |
| NZ_JODL01000019.1; | | | | | |
| 839; 840; 31/32 | | | | | |
| 421; 652698054; | 1748 | 2934 | 4159 | n/a | n/a |
| NZ_KI912610.1; | | | | | |
| 841; 842; 26/27 | | | | | |
| 422; 652698054; | 1750 | 2936 | 4159 | n/a | n/a |
| NZ_KI912610.1; | | | | | |
| 843; 844; 26/27 | | | | | |
| 423; 756828038; | 2050 | 3381 | 4403 | n/a | n/a |
| NZ_CCNC01000143.1; | | | | | |
| 845; 846; 26/27 | | | | | |
| 424; 662140302; | 2135 | 3356 | 3988 | n/a | n/a |
| NZ_JMUB01000087.1; | | | | | |
| 847; 848; 22/23 | | | | | |
| 425; 751285871; | 2224 | 3342 | 4382 | n/a | n/a |
| NZ_CCNA01000001.1; | | | | | |
| 849; 850; 26/27 | | | | | |
| 426; 662140302; | n/a | 2348 | 3763 | n/a | n/a |
| NZ_JMUB01000087.1; | | | | | |
| 851; 852; 22/23 | | | | | |
| 427; 751292755; | n/a | 3343 | 4381 | n/a | n/a |
| NZ_CCNE01000004.1; | | | | | |
| 853; 854; 26/27 | | | | | |
| 428; 970574347; | n/a | 3419 | 4418 | n/a | n/a |
| NZ_LNZF01000001.1; | | | | | |
| 855; 856; 20/21 | | | | | |
| 429; 484099183; | 1721 | 2880 | 4126 | n/a | n/a |
| NZ_AJTY01001072.1; | | | | | |
| 857; 858; 19/20 | | | | | |
| 430; 484099183; | n/a | 3324 | n/a | n/a | n/a |
| NZ AJTY01001072.1; | | | | | |
| 859; 860; 19/20 | | | | | |
| 431; 751265275; | n/a | 3340 | 4380 | n/a | n/a |
| NZ_CCMY01000220.1; | | | | | |
| 861; 862; 26/27 | | | | | |
| 432; 662140302; | 2189 | 3079 | 4240 | n/a | n/a |
| NZ_JMUB01000087.1; | | | | | |
| 863; 864; 22/23 | | | | | |
| 433; 428296779; | n/a | 2764 | 4053 | n/a | n/a |
| NC_019751.1; | | | | | |
| 865; 866; 21/22 | | | | | |
| 434; 662140302; | 2162 | 3075 | 4240 | n/a | n/a |
| NZ_JMUB01000087.1; | | | | | |
| 867; 868; 22/23 | | | | | |
| 435; 563312125; | 1319 | 2340 | n/a | n/a | n/a |
| AYTZ01000052.1; | | | | | |
| 869; 870; 31/32 | | | | | |
| 436; 357028583; | n/a | 2621 | 3936 | n/a | n/a |
| NZ_AGSN01000187.1; | | | | | |
| 871; 872; 26/27 | | | | | |
| 437; 655569633; | 1971 | 3057 | 4491 | n/a | n/a |
| NZ_JIAI01000002.1; | | | | | |
| 873; 874; 32/33 | | | | | |
| 438; 655569633; | 1971 | 3057 | 4491 | n/a | n/a |
| NZ_JIAI01000002.1; | | | | | |
| 875; 876; 43/44 | | | | | |
| 439; 655569633; | 1971 | 3057 | 4491 | n/a | n/a |
| NZ_JIAI01000002.1; | | | | | |
| 877; 878; 32/33 | | | | | |
| 440; 970574347; | 2017 | 3330 | 4373 | n/a | n/a |
| NZ_LNZF01000001.1; | | | | | |
| 879; 880; 20/21 | | | | | |
| 441; 482849861; | 1563 | 2656 | 3963 | n/a | n/a |
| NZ_AKBU01000001.1; | | | | | |
| 881; 882; 3/4 | | | | | |
| 442; 482849861; | 1506 | 2779 | 3985 | n/a | n/a |
| NZ_AKBU01000001.1; | | | | | |
| 883; 884; 3/4 | | | | | |
| 443; 737350949; | 1945 | 3198 | 4328 | n/a | n/a |
| NZ_APVL01000034.1; | | | | | |
| 885; 886; 27/28 | | | | | |
| 444; 482849861; | 1590 | 2689 | 3985 | n/a | n/a |
| NZ_AKBU01000001.1; | | | | | |
| 887; 888; 3/4 | | | | | |
| 445; 671546962; | n/a | 3131 | n/a | n/a | n/a |
| NZ_KL370786.1; | | | | | |
| 889; 890; 33/34 | | | | | |
| 446; 652698054; | 1346 | 2379 | 3788 | n/a | n/a |
| NZ_KI912610.1; | | | | | |
| 891; 892; 26/27 | | | | | |
| 447; 808064534; | 2088 | 3445 | 4433 | n/a | n/a |
| NZ_KQ040798.1; | | | | | |
| 893; 894; 17/18 | | | | | |
| 448; 808051893; | 2088 | 3445 | 4433 | n/a | n/a |
| NZ_KQ040793.1; | | | | | |
| 895; 896; 17/18 | | | | | |
| 449; 808051893; | 2088 | 3445 | 4433 | n/a | n/a |
| NZ_KQ040793.1; | | | | | |
| 897; 898; 10/11 | | | | | |
| 450; 808051893; | 2088 | 3445 | 4433 | n/a | n/a |
| NZ_KQ040793.1; | | | | | |
| 899; 900; 11/12 | | | | | |
| 451; 484016872; | n/a | 2828 | n/a | n/a | n/a |
| NZ_ANAY01000016.1; | | | | | |
| 901; 902; 27/28 | | | | | |
| 452; 736629899; | n/a | 3185 | 4322 | n/a | n/a |
| NZ_JOTN01000004.1; | | | | | |
| 903; 904; 19/20 | | | | | |
| 453; 483219562; | 1698 | 2850 | 4104 | n/a | n/a |
| NZ_KB901875.1; | | | | | |
| 905; 906; 43/44 | | | | | |
| 454; 375307420; | 1542 | 2632 | 3945 | n/a | n/a |
| NZ_JH601049.1; | | | | | |
| 907; 908; 20/21 | | | | | |
| 455; 664540649; | 1898 | 3124 | 4293 | n/a | n/a |
| NZ_JOAX01000009.1; | | | | | |
| 909; 910; 21/22 | | | | | |
| 456; 765315585; | 2075 | 3417 | 4417 | n/a | n/a |
| NZ_LN812103.1; | | | | | |
| 911; 912; 27/28 | | | | | |
| 457; 765315585; | 2075 | 3417 | 4417 | n/a | n/a |
| NZ_LN812103.1; | | | | | |
| 913; 914; 19/20 | | | | | |
| 458; 484099183; | 1771 | 2976 | 4179 | n/a | n/a |
| NZ_AJTY01001072.1; | | | | | |
| 915; 916; 19/20 | | | | | |
| 459; 647274605; | 1752 | 2948 | 4164 | n/a | n/a |
| NZ_ASSA01000134.1; | | | | | |
| 917; 918; 20/21 | | | | | |
| 460; 970574347; | 1770 | 2974 | 4008 | n/a | n/a |
| NZ_LNZF01000001.1; | | | | | |
| 919; 920; 20/21 | | | | | |
| 461; 970574347; | 1610 | 2717 | 4008 | n/a | n/a |
| NZ_LNZF01000001.1; | | | | | |
| 921; 922; 20/21 | | | | | |
| 462; 749188513; | 2012 | 3318 | 4505 | n/a | n/a |
| NZ_CP009122.1; | | | | | |
| 923; 924; 25/26 | | | | | |
| 463; 749188513; | 2012 | 3318 | 4505 | n/a | n/a |
| NZ_CP009122.1; | | | | | |
| 925; 926; 19/20 | | | | | |
| 464; 647269417; | n/a | 2977 | 4180 | n/a | n/a |
| NZ_ASSB01000031.1; | | | | | |
| 927; 928; 20/21 | | | | | |
| 465; 749188513; | 1350 | 2382 | 3789 | n/a | n/a |
| NZ_CP009122.1; | | | | | |
| 929; 930; 25/26 | | | | | |
| 466; 749188513; | 1350 | 2382 | 3789 | n/a | n/a |
| NZ_CP009122.1; | | | | | |
| 931; 932; 19/20 | | | | | |
| 467; 746717390; | n/a | 3321 | n/a | n/a | n/a |
| NZ_JSEF01000015.1; | | | | | |
| 933; 934; 16/17 | | | | | |
| 468; 738760618; | 1966 | 3221 | 4503 | n/a | n/a |
| NZ_JQCR01000002.1; | | | | | |
| 935; 936; 19/20 | | | | | |
| 469; 647230448; | n/a | 2975 | 4178 | n/a | n/a |
| NZ_ASRY01000102.1; | | | | | |
| 937; 938; 20/21 | | | | | |
| 470; 485067426; | 1714 | 2869 | 4114 | n/a | n/a |
| NZ_KB235914.1; | | | | | |
| 939; 940; 26/27 | | | | | |
| 471; 378759075; | 1522 | 3498 | 3929 | n/a | n/a |
| NZ_AFXE01000029.1; | | | | | |
| 941; 942; 22/23 | | | | | |
| 472; 924434005; | 1840 | 3071 | 4238 | n/a | n/a |
| LIYK01000027.1; | | | | | |
| 943; 944; 20/21 | | | | | |
| 473; 647274605; | 1772 | 2978 | 4181 | n/a | n/a |
| NZ_ASSA01000134.1; | | | | | |
| 945; 946; 20/21 | | | | | |
| 474; 152991597; | 1594 | 2693 | 3989 | n/a | n/a |
| NC_009663.1; | | | | | |
| 947; 948; 36/37 | | | | | |
| 475; 647274605; | 2064 | 2716 | 4007 | n/a | n/a |
| NZ_ASSA01000134.1; | | | | | |
| 949; 950; 20/21 | | | | | |
| 476; 751292755; | n/a | 3341 | 4381 | n/a | n/a |
| NZ_CCNE01000004.1; | | | | | |
| 951; 952; 26/27 | | | | | |
| 477; 256419057; | 1602 | 2702 | 3995 | n/a | n/a |
| NC_013132.1; | | | | | |
| 953; 954; 27/28 | | | | | |
| 478; 256419057; | 1602 | 2702 | 3995 | n/a | n/a |
| NC_013132.1; | | | | | |
| 955; 956; 27/28 | | | | | |
| 479; 806905234; | 2236 | 3443 | 4432 | n/a | n/a |
| NZ_LARW01000040.1; | | | | | |
| 957; 958; 11/12 | | | | | |
| 480; 663372343; | 1860 | 3086 | 4256 | n/a | n/a |
| NZ_JOFL01000022.1; | | | | | |
| 959; 960; 44/45 | | | | | |
| 481; 808064534; | 2089 | 3622 | 4434 | n/a | n/a |
| NZ_KQ040798.1; | | | | | |
| 961; 962; 10/11 | | | | | |
| 482; 808064534; | 2089 | 3622 | 4434 | n/a | n/a |
| NZ_KQ040798.1; | | | | | |
| 963; 964; 17/18 | | | | | |
| 483; 808064534; | 2089 | 3622 | 4434 | n/a | n/a |
| NZ_KQ040798.1; | | | | | |
| 965; 966; 10/11 | | | | | |
| 484; 808064534; | 2089 | 3622 | 4434 | n/a | n/a |
| NZ_KQ040798.1; | | | | | |
| 967; 968; 17/18 | | | | | |
| 485; 566226100; | 1422 | 2477 | 3853 | n/a | n/a |
| AZLX01000058.1; | | | | | |
| 969; 970; 27/28 | | | | | |
| 486; 662097244; | 1846 | 3078 | 4244 | n/a | n/a |
| NZ_KL575165.1; | | | | | |
| 971; 972; 20/21 | | | | | |
| 487; 647274605; | 1823 | 3045 | 4181 | n/a | n/a |
| NZ_ASSA01000134.1; | | | | | |
| 973; 974; 20/21 | | | | | |
| 488; 924434005; | 2000 | 3306 | 4366 | n/a | n/a |
| LIYK01000027.1; | | | | | |
| 975; 976; 20/21 | | | | | |
| 489; 378759075; | 1522 | 2609 | 3929 | n/a | n/a |
| NZ_AFXE01000029.1; | | | | | |
| 977; 978; 22/23 | | | | | |
| 490; 647274605; | 1752 | 3637 | 4520 | n/a | n/a |
| NZ_ASSA01000134.1; | | | | | |
| 979; 980; 20/21 | | | | | |
| 491; 751299847; | n/a | 3344 | 4381 | n/a | n/a |
| NZ_CCMZ01000015.1; | | | | | |
| 981; 982; 26/27 | | | | | |
| 492; 375307420; | 1576 | 2665 | 3967 | n/a | n/a |
| NZ_JH601049.1; | | | | | |
| 983; 984; 20/21 | | | | | |
| 493; 906344334; | 2131 | 3512 | 4470 | n/a | n/a |
| NZ_LFXA01000002.1; | | | | | |
| 985; 986; 25/26 | | | | | |
| 494; 759948103; | 2063 | 3611 | 4412 | n/a | n/a |
| NZ_JOAG01000045.1; | | | | | |
| 987; 988; 27/28 | | | | | |
| 495; 664478668; | 1895 | 3119 | 4288 | n/a | n/a |
| NZ_JOJI01000002.1; | | | | | |
| 989; 990; 19/20 | | | | | |
| 496; 662043624; | n/a | 3264 | 4241 | n/a | n/a |
| NZ_JNXL01000469.1; | | | | | |
| 991; 992; 22/23 | | | | | |
| 497; 906344334; | 1458 | 2528 | 3874 | n/a | n/a |
| NZ_LFXA01000002.1; | | | | | |
| 993; 994; 25/26 | | | | | |
| 498; 664104387; | 1879 | 3102 | 3924 | n/a | n/a |
| NZ_JOJJ01000005.1; | | | | | |
| 995; 996; 19/20 | | | | | |
| 499; 664104387; | 1862 | 3089 | 4258 | n/a | n/a |
| NZ_JOJJ01000005.1; | | | | | |
| 997; 998; 19/20 | | | | | |
| 500; 664104387; | 1880 | 3104 | 4274 | n/a | n/a |
| NZ_JOJJ1000005.1; | | | | | |
| 999; 1000; 19/20 | | | | | |
| 501; 664565137; | 1900 | 3605 | 4511 | n/a | n/a |
| NZ_KL591029.1; | | | | | |
| 1001; 1002; 19/20 | | | | | |
| 502; 664104387; | 1466 | 2537 | 3881 | n/a | n/a |
| NZ_JOJJ01000005.1; | | | | | |
| 1003; 1004; 19/20 | | | | | |
| 503; 664104387; | 1462 | 2533 | 3877 | n/a | n/a |
| NZ_JOJJ01000005.1; | | | | | |
| 1005; 1006; 19/20 | | | | | |
| 504; 664104387; | 1515 | 3669 | 3924 | n/a | n/a |
| NZ_JOJJ01000005.1; | | | | | |
| 1007; 1008; 19/20 | | | | | |
| 505; 664104387; | 1515 | 3161 | 4307 | n/a | n/a |
| NZ_JOJJ01000005.1; | | | | | |
| 1009; 1010; 19/20 | | | | | |
| 506; 664104387; | 1515 | 2600 | 3924 | n/a | n/a |
| NZ_JOJJ01000005.1; | | | | | |
| 1011; 1012; 19/20 | | | | | |
| 507; 664323078; | 1891 | 3111 | 4283 | n/a | n/a |
| NZ_JOIB01000032.1; | | | | | |
| 1013; 1014; 19/20 | | | | | |
| 508; 315499382; | 2137 | 2723 | n/a | n/a | n/a |
| NC_014817.1; | | | | | |
| 1015; 1016; 25/26 | | | | | |
| 509; 315499382; | 2137 | 2723 | n/a | n/a | n/a |
| NC_014817.1; | | | | | |
| 1017; 1018; 25/26 | | | | | |
| 510; 664066234; | 2263 | 3658 | 4272 | n/a | n/a |
| NZ_JOES01000124.1; | | | | | |
| 1019; 1020; 19/20 | | | | | |
| 511; 740092143; | n/a | 3585 | 4358 | n/a | n/a |
| NZ_JFCB01000064.1; | | | | | |
| 1021; 1022; 19/20 | | | | | |
| 512; 930029075; | 2276 | 3677 | n/a | n/a | n/a |
| NZ_LJHO01000007.1; | | | | | |
| 1023; 1024; 18/19 | | | | | |
| 513; 664104387; | 1515 | 3100 | 4273 | n/a | n/a |
| NZ_JOJJ01000005.1; | | | | | |
| 1025; 1026; 19/20 | | | | | |
| 514; 664104387; | 1515 | 3127 | 4258 | n/a | n/a |
| NZ_JOJJ01000005.1; | | | | | |
| 1027; 1028; 19/20 | | | | | |
| 515; 664104387; | 1464 | 2535 | 3879 | n/a | n/a |
| NZ_JOJJ01000005.1; | | | | | |
| 1029; 1030; 19/20 | | | | | |
| 516; 902792184; | n/a | 3511 | 4469 | n/a | n/a |
| NZ_LFVW01000692.1; | | | | | |
| 1031; 1032; 22/23 | | | | | |
| 517; 485125031; | 2161 | 3553 | 4378 | n/a | n/a |
| NZ_BAGL01000055.1; | | | | | |
| 1033; 1034; 18/19 | | | | | |
| 518; 759934284; | 2223 | 3607 | 4410 | n/a | n/a |
| NZ_JOAG01000009.1; | | | | | |
| 1035; 1036; 23/24 | | | | | |
| 519; 759934284; | 2223 | 3607 | 4410 | n/a | n/a |
| NZ_JOAG01000009.1; | | | | | |
| 1037; 1038; 23/24 | | | | | |
| 520; 746288194; | 2004 | 3310 | n/a | n/a | n/a |
| NZ_JRVC01000013.1; | | | | | |
| 1039; 1040; 22/23 | | | | | |
| 521; 664194528; | n/a | 2389 | n/a | n/a | n/a |
| NZ_JOIG01000002.1; | | | | | |
| 1041; 1042; 23/24 | | | | | |
| 522; 664194528; | n/a | 3455 | n/a | n/a | n/a |
| NZ_JOIG01000002.1; | | | | | |
| 1043; 1044; 23/24 | | | | | |
| 523; 664066234; | 1877 | 3099 | 4272 | n/a | n/a |
| NZ_JOES01000124.1; | | | | | |
| 1045; 1046; 19/20 | | | | | |
| 524; 664066234; | 1468 | 2539 | 3883 | n/a | n/a |
| NZ_JOES01000124.1; | | | | | |
| 1047; 1048; 19/20 | | | | | |
| 525; 72160406; | 1584 | 2676 | 3975 | n/a | n/a |
| NC_007333.1; | | | | | |
| 1049; 1050; 22/23 | | | | | |
| 526; 926371520; | n/a | 3657 | 4528 | n/a | n/a |
| NZ_LGCW01000274.1; | | | | | |
| 1051; 1052; 27/28 | | | | | |
| 527; 664244706; | 1887 | 3577 | 4278 | n/a | n/a |
| NZ_JOBD01000002.1; | | | | | |
| 1053; 1054; 27/28 | | | | | |
| 528; 739594477; | 1973 | 3236 | n/a | n/a | n/a |
| NZ_JFHR01000025.1; | | | | | |
| 1055; 1056; 22/23 | | | | | |
| 529; 808402906; | 1376 | 2422 | n/a | n/a | n/a |
| CCBH010000144.1; | | | | | |
| 1057; 1058; 23/24 | | | | | |
| 530; 746242072; | 2217 | 3308 | n/a | n/a | n/a |
| NZ_JIDI01000011.1; | | | | | |
| 1059; 1060; 23/24 | | | | | |
| 531; 72160406; | 1584 | 2790 | 3975 | n/a | n/a |
| NC_007333.1; | | | | | |
| 1061; 1062; 22/23 | | | | | |
| 532; 664194528; | n/a | 3106 | n/a | n/a | n/a |
| NZ_JOIG01000002.1; | | | | | |
| 1063; 1064; 23/24 | | | | | |
| 533; 483527356; | 1709 | 2863 | n/a | n/a | n/a |
| NZ_BARE01000016.1; | | | | | |
| 1065; 1066; 22/23 | | | | | |
| 534; 936191447; | n/a | 3687 | n/a | n/a | n/a |
| NZ_LBLZ01000002.1; | | | | | |
| 1067; 1068; 22/23 | | | | | |
| 535; 484226753; | 1692 | 2843 | n/a | n/a | n/a |
| NZ_AQWM01000013.1; | | | | | |
| 1069; 1070; 21/22 | | | | | |
| 536; 664104387; | 1465 | 2536 | 3880 | n/a | n/a |
| NZ_JOJJ01000005.1; | | | | | |
| 1071; 1072; 19/20 | | | | | |
| 537; 484227180; | 1694 | 2845 | 4101 | n/a | n/a |
| NZ_AQWO01000002.1; | | | | | |
| 1073; 1074; 18/19 | | | | | |
| 538; 664104387; | 1515 | 3667 | 3924 | n/a | n/a |
| NZ_JOJJ01000005.1; | | | | | |
| 1075; 1076; 19/20 | | | | | |
| 539; 936191447; | n/a | 2399 | n/a | n/a | n/a |
| NZ_LBLZ01000002.1; | | | | | |
| 1077; 1078; 22/23 | | | | | |
| 540; 484113405; | 1730 | 2895 | n/a | n/a | n/a |
| NZ_BACX01000237.1; | | | | | |
| 1079; 1080; 23/24 | | | | | |
| 541; 664063830; | 1990 | 3571 | 4497 | n/a | n/a |
| NZ_JODT01000002.1; | | | | | |
| 1081; 1082; 28/29 | | | | | |
| 542; 451338568; | 1530 | 2617 | 3932 | n/a | n/a |
| NZ_ANMG01000060.1; | | | | | |
| 1083; 1084; 18/19 | | | | | |
| 543; 544819688; | 1728 | 2892 | n/a | n/a | n/a |
| NZ_AIHL01000147.1; | | | | | |
| 1085; 1086; 18/19 | | | | | |
| 544; 557833377; | 1742 | 2910 | n/a | n/a | n/a |
| NZ_AWGE01000008.1; | | | | | |
| 1087; 1088; 20/21 | | | | | |
| 545; 557833377; | 1742 | 2910 | n/a | n/a | n/a |
| NZ_AWGE01000008.1; | | | | | |
| 1089; 1090; 22/23 | | | | | |
| 546; 347526385; | 1625 | 2743 | n/a | n/a | n/a |
| NC_015976.1; | | | | | |
| 1091; 1092; 21/22 | | | | | |
| 547; 334133217; | 2031 | 2732 | n/a | n/a | n/a |
| NC_015579.1; | | | | | |
| 1093; 1094; 23/24 | | | | | |
| 548; 746241774; | 2002 | 3594 | n/a | n/a | n/a |
| NZ_JTDI01000009.1; | | | | | |
| 1095; 1096; 24/25 | | | | | |
| 549; 659864921; | 1843 | 3074 | n/a | n/a | n/a |
| NZ_JONW01000006.1; | | | | | |
| 1097; 1098; 20/21 | | | | | |
| 550; 659864921; | 1843 | 3074 | n/a | n/a | n/a |
| NZ_JONW01000006.1; | | | | | |
| 1099; 1100; 20/21 | | | | | |
| 551; 294023656; | 1608 | 2709 | n/a | n/a | n/a |
| NC_014007.1; | | | | | |
| 1101; 1102; 23/24 | | | | | |
| 552; 749321911; | 1765 | 2966 | n/a | n/a | n/a |
| NZ_CP006644.1; | | | | | |
| 1103; 1104; 18/19 | | | | | |
| 553; 739630357; | 1977 | 3559 | n/a | n/a | n/a |
| NZ_JFYY01000027.1; | | | | | |
| 1105; 1106; 21/22 | | | | | |
| 554; 739622900; | 1975 | 3240 | n/a | n/a | n/a |
| NZ_JPPQ01000069.1; | | | | | |
| 1107; 1108; 12/13 | | | | | |
| 555; 663365281; | n/a | 3589 | 4255 | n/a | n/a |
| NZ_JODN01000094.1; | | | | | |
| 1109; 1110; 22/23 | | | | | |
| 556; 484226810; | 1693 | 2844 | n/a | n/a | n/a |
| NZ_AQWM01000032.1; | | | | | |
| 1111; 1112; 24/25 | | | | | |
| 557; 759429528; | 2177 | 3387 | n/a | n/a | n/a |
| NZ_JEMV01000036.1; | | | | | |
| 1113; 1114; 23/24 | | | | | |
| 558; 654975403; | 2173 | 3043 | 4486 | n/a | n/a |
| NZ_KI601366.1; | | | | | |
| 1115; 1116; 27/28 | | | | | |
| 559; 541476958; | 1729 | 3334 | 4375 | n/a | n/a |
| AWSB01000006.1; | | | | | |
| 1117; 1118; 58/59 | | | | | |
| 560; 484207511; | 1720 | 2879 | 4125 | n/a | n/a |
| NZ_AQUZ01000008.1; | | | | | |
| 1119; 1120; 20/21 | | | | | |
| 561; 484867900; | n/a | 2864 | n/a | n/a | n/a |
| NZ_AGNH01000612.1; | | | | | |
| 1121; 1122; 15/16 | | | | | |
| 562; 544811486; | 1908 | 2891 | n/a | n/a | n/a |
| NZ_ATDP01000107.1; | | | | | |
| 1123; 1124; 17/18 | | | | | |
| 563; 783211546; | 2085 | 3439 | 4428 | n/a | n/a |
| NZ_JZKH01000064.1; | | | | | |
| 1125; 1126; 30/31 | | | | | |
| 564; 873296042; | 2116 | 3488 | n/a | n/a | n/a |
| NZ_LECE01000021.1; | | | | | |
| 1127; 1128; 14/15 | | | | | |
| 565; 651281457; | 1937 | 3557 | 4489 | n/a | n/a |
| NZ_JADG01000010.1; | | | | | |
| 1129; 1130; 20/21 | | | | | |
| 566; 664348063; | n/a | 3495 | 4465 | n/a | n/a |
| NZ_JOFN01000002.1; | | | | | |
| 1131; 1132; 29/30 | | | | | |
| 567; 893711343; | 2123 | 3246 | n/a | n/a | n/a |
| NZ_KQ235994.1; | | | | | |
| 1133; 1134; 12/13 | | | | | |
| 568; 893711343; | 2123 | 3499 | n/a | n/a | n/a |
| NZ_KQ235994.1; | | | | | |
| 1135; 1136; 12/13 | | | | | |
| 569; 663365281; | n/a | 3576 | 4255 | n/a | n/a |
| NZ_JODN01000094.1; | | | | | |
| 1137; 1138; 22/23 | | | | | |
| 570; 739661773; | 1980 | 3587 | n/a | n/a | n/a |
| NZ_JGVR01000002.1; | | | | | |
| 1139; 1140; 13/14 | | | | | |
| 571; 739661773; | 1978 | 2608 | n/a | n/a | n/a |
| NZ_JGVR01000002.1; | | | | | |
| 1141; 1142; 13/14 | | | | | |
| 572; 749188513; | 1349 | 2381 | n/a | n/a | n/a |
| NZ_CP009122.1; | | | | | |
| 1143; 1144; 23/24 | | | | | |
| 573; 734983422; | 1932 | 3181 | n/a | n/a | n/a |
| NZ_JSXI01000079.1; | | | | | |
| 1145; 1146; 18/19 | | | | | |
| 574; 930029077; | 2277 | 3678 | n/a | n/a | n/a |
| NZ_LJHO01000009.1; | | | | | |
| 1147; 1148; 22/23 | | | | | |
| 575; 664556736; | 1899 | 3604 | 4294 | n/a | n/a |
| NZ_KL591003.1; | | | | | |
| 1149; 1150; 40/41 | | | | | |
| 576; 739701660; | 1984 | 3249 | n/a | n/a | n/a |
| NZ_JNFC01000024.1; | | | | | |
| 1151; 1152; 20/21 | | | | | |
| 577; 737322991; | 2200 | 3195 | n/a | n/a | n/a |
| NZ_JMQR01000005.1; | | | | | |
| 1153; 1154; 20/21 | | | | | |
| 578; 737322991; | 2200 | 3195 | n/a | n/a | n/a |
| NZ_JMQR01000005.1; | | | | | |
| 1155; 1156; 20/21 | | | | | |
| 579; 557839256; | 1744 | 2912 | n/a | n/a | n/a |
| NZ_AWGF01000005.1; | | | | | |
| 1157; 1158; 24/25 | | | | | |
| 580; 737322991; | 1437 | 2499 | n/a | n/a | n/a |
| NZ_JMQR01000005.1; | | | | | |
| 1159; 1160; 20/21 | | | | | |
| 581; 737322991; | 1437 | 2499 | n/a | n/a | n/a |
| NZ_JMQR01000005.1; | | | | | |
| 1161; 1162; 20/21 | | | | | |
| 582; 783211546; | 2086 | 3621 | 4429 | n/a | n/a |
| NZ_JZKH01000064.1; | | | | | |
| 1163; 1164; 30/31 | | | | | |
| 583; 893711364; | 2124 | 3500 | n/a | n/a | n/a |
| NZ_KQ236015.1; | | | | | |
| 1165; 1166; 21/22 | | | | | |
| 584; 543418148; | 1429 | 2487 | n/a | n/a | n/a |
| BATC01000005.1; | | | | | |
| 1167; 1168; 26/27 | | | | | |
| 585; 797049078; | 2269 | 3666 | 4536 | n/a | n/a |
| JZWX01001028.1; | | | | | |
| 1169; 1170; 25/26 | | | | | |
| 586; 893711364; | 1979 | 3244 | n/a | n/a | n/a |
| NZ_KQ236015.1; | | | | | |
| 1171; 1172; 21/22 | | | | | |
| 587; 327367349; | 1335 | 2361 | n/a | n/a | n/a |
| CP002599.1; | | | | | |
| 1173; 1174; 27/28 | | | | | |
| 588; 494022722; | 1539 | 3242 | n/a | n/a | n/a |
| NZ_CAVK010000217.1; | | | | | |
| 1175; 1176; 21/22 | | | | | |
| 589; 893711343; | 1457 | 2527 | n/a | n/a | n/a |
| NZ_KQ235994.1; | | | | | |
| 1177; 1178; 12/13 | | | | | |
| 590; 930473294; | 2278 | 3680 | 4540 | n/a | n/a |
| NZ_LJCV01000275.1; | | | | | |
| 1179; 1180; 36/37 | | | | | |
| 591; 514419386; | 1827 | 2894 | n/a | n/a | n/a |
| NZ_KE148338.1; | | | | | |
| 1181; 1182; 22/23 | | | | | |
| 592; 930473294; | 1472 | 2546 | 3888 | n/a | n/a |
| NZ_LJCV01000275.1; | | | | | |
| 1183; 1184; 36/37 | | | | | |
| 593; 893711364; | 1521 | 2607 | n/a | n/a | n/a |
| NZ_KQ236015.1; | | | | | |
| 1185; 1186; 21/22 | | | | | |
| 594; 483682977; | 1700 | 2852 | 4483 | n/a | n/a |
| NZ_KB904636.1; | | | | | |
| 1187; 1188; 29/30 | | | | | |
| 595; 893711364; | 1546 | 2637 | n/a | n/a | n/a |
| NZ_KQ236015.1; | | | | | |
| 1189; 1190; 21/22 | | | | | |
| 596; 914607448; | 2148 | 3539 | n/a | n/a | n/a |
| NZ_JYNE01000028.1; | | | | | |
| 1191; 1192; 22/23 | | | | | |
| 597; 753809381; | n/a | 2967 | n/a | n/a | n/a |
| NZ_CP006850.1; | | | | | |
| 1193; 1194; 23/24 | | | | | |
| 598; 759941310; | n/a | n/a | n/a | 3608 | n/a |
| NZ_JOAG01000020.1; | | | | | |
| 1195; 1196; 30/31 | | | | | |
| 599; 484023808; | n/a | 2833 | 4092 | n/a | n/a |
| NZ_ANBF01000204.1; | | | | | |
| 1197; 1198; 22/23 | | | | | |
| 600; 763095630; | 2067 | 3405 | n/a | n/a | n/a |
| NZ_JXZE01000009.1; | | | | | |
| 1199; 1200; 23/24 | | | | | |
| 601; 797049078; | 1471 | 2543 | 3886 | n/a | n/a |
| JZWX01001028.1; | | | | | |
| 1201; 1202; 25/26 | | | | | |
| 602; 663818579; | 1867 | 3095 | n/a | n/a | n/a |
| NZ_JNAC01000042.1; | | | | | |
| 1203; 1204; 23/24 | | | | | |
| 603; 541476958; | 1414 | 2468 | 3846 | n/a | n/a |
| AWSB01000006.1; | | | | | |
| 1205; 1206; 58/59 | | | | | |
| 604; 663300941; | 1857 | 3083 | 4253 | n/a | n/a |
| NZ_JNZY01000037.1; | | | | | |
| 1207; 1208; 25/26 | | | | | |
| 605; 196476886; | 1325 | 2350 | n/a | n/a | n/a |
| CP000747.1; | | | | | |
| 1209; 1210; 23/24 | | | | | |
| 606; 797049078; | 1455 | 2524 | 3872 | n/a | n/a |
| JZWX01001028.1; | | | | | |
| 1211; 1212; 25/26 | | | | | |
| 607; 402821166; | 1555 | 2645 | n/a | n/a | n/a |
| NZ_ALVC01000003.1; | | | | | |
| 1213; 1214; 23/24 | | | | | |
| 608; 763095630; | 1451 | 2515 | n/a | n/a | n/a |
| NZ_JXZE01000009.1; | | | | | |
| 1215; 1216; 23/24 | | | | | |
| 609; 483996974; | 1675 | 2817 | n/a | n/a | n/a |
| NZ_AMYX01000026.1; | | | | | |
| 1217; 1218; 21/22 | | | | | |
| 610; 759944490; | 2062 | 3610 | 4411 | n/a | n/a |
| NZ_JOAG01000030.1; | | | | | |
| 1219; 1220; 26/27 | | | | | |
| 611; 269095543; | 1327 | 2352 | 3764 | n/a | n/a |
| CP001819.1; | | | | | |
| 1221; 1222; 13/14 | | | | | |
| 612; 393773868; | 2060 | 2647 | n/a | n/a | n/a |
| NZ_AKFJ01000097.1; | | | | | |
| 1223; 1224; 18/19 | | | | | |
| 613; 765344939; | 1982 | 2657 | n/a | n/a | n/a |
| NZ_CP010954.1; | | | | | |
| 1225; 1226; 22/23 | | | | | |
| 614; 873296295; | n/a | 3490 | n/a | n/a | n/a |
| NZ_LECE01000071.1; | | | | | |
| 1227; 1228; 23/24 | | | | | |
| 615; 759431957; | 2053 | 3388 | n/a | n/a | n/a |
| NZ_JEMV01000094.1; | | | | | |
| 1229; 1230; 12/13 | | | | | |
| 616; 765344939; | 2076 | 3421 | n/a | n/a | n/a |
| NZ_CP010954.1; | | | | | |
| 1231; 1232; 22/23 | | | | | |
| 617; 262193326; | 1603 | 2703 | n/a | n/a | n/a |
| NC_013440.1; | | | | | |
| 1233; 1234; 24/25 | | | | | |
| 618; 329889017; | 1508 | 2591 | n/a | n/a | n/a |
| NZ_GL883086.1; | | | | | |
| 1235; 1236; 19/20 | | | | | |
| 619; 664428976; | 1854 | 3116 | 4250 | n/a | n/a |
| NZ_KL585179.1; | | | | | |
| 1237; 1238; 21/22 | | | | | |
| 620; 764364074; | 2230 | 3407 | n/a | n/a | n/a |
| NZ_CP010836.1; | | | | | |
| 1239; 1240; 22/23 | | | | | |
| 621; 764364074; | 2230 | 3407 | n/a | n/a | n/a |
| NZ_CP010836.1; | | | | | |
| 1241; 1242; 19/20 | | | | | |
| 622; 402821307; | 2183 | 3219 | n/a | n/a | n/a |
| NZ_ALVC01000008.1; | | | | | |
| 1243; 1244; 12/13 | | | | | |
| 623; 484115568; | 1775 | 2985 | n/a | n/a | n/a |
| NZ_BACX01000797.1; | | | | | |
| 1245; 1246; 22/23 | | | | | |
| 624; 402821307; | 1556 | 2646 | n/a | n/a | n/a |
| NZ_ALVC01000008.1; | | | | | |
| 1247; 1248; 12/13 | | | | | |
| 625; 386845069; | 1633 | 3599 | 4037 | n/a | n/a |
| NC_017803.1; | | | | | |
| 1249; 1250; 22/23 | | | | | |
| 626; 386845069; | 1339 | 2366 | 3773 | n/a | n/a |
| NC_017803.1; | | | | | |
| 1251; 1252; 22/23 | | | | | |
| 627; 347526385; | n/a | 2742 | n/a | n/a | n/a |
| NC_015976.1; | | | | | |
| 1253; 1254; 12/13 | | | | | |
| 628; 696542396; | 2207 | 3163 | n/a | n/a | n/a |
| NZ_JQFJ01000002.1; | | | | | |
| 1255; 1256; 20/21 | | | | | |
| 629; 702914619; | 1926 | 3168 | 4312 | n/a | n/a |
| NZ_JNXI01000006.1; | | | | | |
| 1257; 1258; 25/26 | | | | | |
| 630; 602262270; | 1427 | 2484 | 3857 | n/a | n/a |
| JENI01000029.1; | | | | | |
| 1259; 1260; 21/22 | | | | | |
| 631; 739629085; | 1976 | 3241 | n/a | n/a | n/a |
| NZ_JFYY01000016.1; | | | | | |
| 1261; 1262; 23/24 | | | | | |
| 632; 602262270; | 1956 | 3213 | 3980 | n/a | n/a |
| JENI01000029.1; | | | | | |
| 1263; 1264; 21/22 | | | | | |
| 633; 602262270; | n/a | 2683 | 3980 | n/a | n/a |
| JENI01000029.1; | | | | | |
| 1265; 1266; 21/22 | | | | | |
| 634; 602262270; | 1421 | 2476 | 3852 | n/a | n/a |
| JENI01000029.1; | | | | | |
| 1267; 1268; 21/22 | | | | | |
| 635; 659889283; | 1844 | 3253 | n/a | n/a | n/a |
| NZ_JOOE01000001.1; | | | | | |
| 1269; 1270; 18/19 | | | | | |
| 636; 737322991; | 2201 | 3196 | n/a | n/a | n/a |
| NZ_JMQR01000005.1; | | | | | |
| 1271; 1272; 19/20 | | | | | |
| 637; 444405902; | 1509 | 2592 | n/a | n/a | n/a |
| NZ_KB291784.1; | | | | | |
| 1273; 1274; 20/21 | | | | | |
| 638; 444405902; | 1509 | 2592 | n/a | n/a | n/a |
| NZ_KB291784.1; | | | | | |
| 1275; 1276; 20/21 | | | | | |
| 639; 602262270; | 1956 | 3210 | 3980 | n/a | n/a |
| JENI01000029.1; | | | | | |
| 1277; 1278; 21/22 | | | | | |
| 640; 546154317; | 1415 | 2469 | 3847 | n/a | n/a |
| NZ_ACVN02000045.1; | | | | | |
| 1279; 1280; 18/19 | | | | | |
| 641; 602262270; | 1956 | 3212 | 4333 | n/a | n/a |
| JENI01000029.1; | | | | | |
| 1281; 1282; 21/22 | | | | | |
| 642; 938956730; | 2284 | 3693 | n/a | n/a | n/a |
| NZ_CP009429.1; | | | | | |
| 1283; 1284; 20/21 | | | | | |
| 643; 602262270; | 1439 | 2501 | 3862 | n/a | n/a |
| JENI01000029.1; | | | | | |
| 1285; 1286; 21/22 | | | | | |
| 644; 737323704; | n/a | 3197 | n/a | n/a | n/a |
| NZ_JMQR01000012.1; | | | | | |
| 1287; 1288; 19/20 | | | | | |
| 645; 737323704; | n/a | 3197 | n/a | n/a | n/a |
| NZ_JMQR01000012.1; | | | | | |
| 1289; 1290; 18/19 | | | | | |
| 646; 602262270; | 1441 | 2503 | 3863 | n/a | n/a |
| JENI01000029.1; | | | | | |
| 1291; 1292; 21/22 | | | | | |
| 647; 657605746; | 1836 | 3067 | n/a | n/a | n/a |
| NZ_JNIX01000010.1; | | | | | |
| 1293; 1294; 18/19 | | | | | |
| 648; 647728918; | 1774 | 2980 | n/a | n/a | n/a |
| NZ_JHOF01000018.1; | | | | | |
| 1295; 1296; 19/20 | | | | | |
| 649; 938989745; | 2288 | 3697 | n/a | n/a | n/a |
| NZ_CP012897.1; | | | | | |
| 1297; 1298; 20/21 | | | | | |
| 650; 938989745; | 2288 | 3697 | n/a | n/a | n/a |
| NZ_CP012897.1; | | | | | |
| 1299; 1300; 19/20 | | | | | |
| 651; 664434000; | n/a | 3118 | n/a | n/a | n/a |
| NZ_JOIA01001078.1; | | | | | |
| 1301; 1302; 21/22 | | | | | |
| 652; 703243990; | n/a | 3588 | n/a | n/a | n/a |
| NZ_JNYM01001430.1; | | | | | |
| 1303; 1304; 20/21 | | | | | |
| 653; 739699072; | 1983 | 3248 | n/a | n/a | n/a |
| NZ_JNFC01000001.1; | | | | | |
| 1305; 1306; 19/20 | | | | | |
| 654; 739699072; | 1983 | 3248 | n/a | n/a | n/a |
| NZ_JNFC01000001.1; | | | | | |
| 1307; 1308; 19/20 | | | | | |
| 655; 739699072; | 1983 | 3319 | n/a | n/a | n/a |
| NZ_JNFC01000001.1; | | | | | |
| 1309; 1310; 19/20 | | | | | |
| 656; 739699072; | 1983 | 3319 | n/a | n/a | n/a |
| NZ_JNFC01000001.1; | | | | | |
| 1311; 1312; 19/20 | | | | | |
| 657; 343957487; | 1573 | 2662 | n/a | n/a | n/a |
| NZ_AEWF01000005.1; | | | | | |
| 1313; 1314; 31/32 | | | | | |
| 658; 343957487; | 1573 | 2662 | n/a | n/a | n/a |
| NZ_AEWF01000005.1; | | | | | |
| 1315; 1316; 31/32 | | | | | |
| 659; 938154362; | 1364 | 2401 | n/a | n/a | n/a |
| CP009430.1; | | | | | |
| 1317; 1318; 23/24 | | | | | |
| 660; 566155502; | 1746 | 2914 | 4151 | n/a | n/a |
| NZ_CM002285.1; | | | | | |
| 1319; 1320; 37/38 | | | | | |
| 661; 399903251; | n/a | 2453 | 3834 | n/a | n/a |
| ALJK01000024.1; | | | | | |
| 1321; 1322; 22/23 | | | | | |
| 662; 399903251; | n/a | 2453 | 3834 | n/a | n/a |
| ALJK01000024.1; | | | | | |
| 1323; 1324; 21/22 | | | | | |
| 663; 399903251; | n/a | 2453 | 3834 | n/a | n/a |
| ALJK01000024.1; | | | | | |
| 1325; 1326; 24/25 | | | | | |
| 664; 763097360; | 2229 | 3617 | n/a | n/a | n/a |
| NZ_JXZE01000017.1; | | | | | |
| 1327; 1328; 21/22 | | | | | |
| 665; 746290581; | 2218 | 3595 | n/a | n/a | n/a |
| NZ_JRVC01000028.1; | | | | | |
| 1329; 1330; 22/23 | | | | | |
| 666; 739287390; | 2206 | 3137 | 4303 | n/a | n/a |
| NZ_JMFA01000010.1; | | | | | |
| 1331; 1332; 21/22 | | | | | |
| 667; 694033726; | 2206 | 3137 | 4303 | n/a | n/a |
| NZ_JMEM01000016.1; | | | | | |
| 1333; 1334; 21/22 | | | | | |
| 668; 739287390; | 2206 | 3137 | 4303 | n/a | n/a |
| NZ_JMFA01000010.1; | | | | | |
| 1335; 1336; 21/22 | | | | | |
| 669; 483997957; | 1677 | 2819 | n/a | n/a | n/a |
| NZ_AMYY01000002.1; | | | | | |
| 1337; 1338; 20/21 | | | | | |
| 670; 898301838; | n/a | 3510 | n/a | n/a | n/a |
| NZ_LAVK01000307.1; | | | | | |
| 1339; 1340; 36/37 | | | | | |
| 671; 739287390; | 2205 | 3138 | 4303 | n/a | n/a |
| NZ_JMFA01000010.1; | | | | | |
| 1341; 1342; 21/22 | | | | | |
| 672; 739287390; | 2205 | 3138 | 4303 | n/a | n/a |
| NZ_JMFA01000010.1; | | | | | |
| 1343; 1344; 21/22 | | | | | |
| 673; 739287390; | 2205 | 3138 | 4303 | n/a | n/a |
| NZ_JMFA01000010.1; | | | | | |
| 1345; 1346; 21/22 | | | | | |
| 674; 739287390; | 2205 | 3230 | 4303 | n/a | n/a |
| NZ_JMFA01000010.1; | | | | | |
| 1347; 1348; 21/22 | | | | | |
| 675; 739287390; | 2205 | 3230 | 4303 | n/a | n/a |
| NZ_JMFA01000010.1; | | | | | |
| 1349; 1350; 21/22 | | | | | |
| 676; 739287390; | 2205 | 3230 | 4303 | n/a | n/a |
| NZ_JMFA01000010.1; | | | | | |
| 1351; 1352; 21/22 | | | | | |
| 677; 766589647; | 1754 | 2950 | 4166 | n/a | n/a |
| NZ_CEHJ01000007.1; | | | | | |
| 1353; 1354; 18/19 | | | | | |
| 678; 938989745; | 2289 | 3698 | n/a | n/a | n/a |
| NZ_CP012897.1; | | | | | |
| 1355; 1356; 20/21 | | | | | |
| 679; 938989745; | 2289 | 3698 | n/a | n/a | n/a |
| NZ_CP012897.1; | | | | | |
| 1357; 1358; 20/21 | | | | | |
| 680; 739610197; | 1974 | 3238 | n/a | n/a | n/a |
| NZ_JFZA02000028.1; | | | | | |
| 1359; 1360; 22/23 | | | | | |
| 681; 766589647; | 2081 | 3430 | 4423 | n/a | n/a |
| NZ_CEHJ01000007.1; | | | | | |
| 1361; 1362; 18/19 | | | | | |
| 682; 896667361; | 2130 | 3509 | 4468 | n/a | n/a |
| NZ_JVGV01000030.1; | | | | | |
| 1363; 1364; 18/19 | | | | | |
| 683; 834156795; | 1435 | 2496 | n/a | n/a | n/a |
| BBRO01000001.1; | | | | | |
| 1365; 1366; 20/21 | | | | | |
| 684; 736736050; | 2184 | 3561 | n/a | n/a | n/a |
| NZ_AWFG01000029.1; | | | | | |
| 1367; 1368; 27/28 | | | | | |
| 685; 766589647; | 1754 | 3424 | 4166 | n/a | n/a |
| NZ_CEHJ01000007.1; | | | | | |
| 1369; 1370; 18/19 | | | | | |
| 686; 938956730; | 1363 | 2400 | n/a | n/a | n/a |
| NZ_CP009429.1; | | | | | |
| 1371; 1372; 19/20 | | | | | |
| 687; 938956730; | 1363 | 2400 | n/a | n/a | n/a |
| NZ_CP009429.1; | | | | | |
| 1373; 1374; 21/22 | | | | | |
| 688; 545327527; | n/a | 2893 | 4376 | n/a | n/a |
| NZ_KE951412.1; | | | | | |
| 1375; 1376; 25/26 | | | | | |
| 689; 545327527; | n/a | 2893 | 4376 | n/a | n/a |
| NZ_KE951412.1; | | | | | |
| 1377; 1378; 13/14 | | | | | |
| 690; 545327527; | n/a | 2893 | 4376 | n/a | n/a |
| NZ_KE951412.1; | | | | | |
| 1379; 1380; 19/20 | | | | | |
| 691; 545327527; | n/a | 2893 | 4376 | n/a | n/a |
| NZ_KE951412.1; | | | | | |
| 1381; 1382; 19/20 | | | | | |
| 692; 541473965; | n/a | 2893 | 4376 | n/a | n/a |
| AWSB01000041.1; | | | | | |
| 1383; 1384; 20/21 | | | | | |
| 693; 896567682; | 2128 | 3507 | n/a | n/a | n/a |
| NZ_JUMH01000022.1; | | | | | |
| 1385; 1386; 16/17 | | | | | |
| 694; 728827031; | 2210 | 3178 | n/a | n/a | n/a |
| NZ_JROG01000008.1; | | | | | |
| 1387; 1388; 20/21 | | | | | |
| 695; 896567682; | 2126 | 3502 | n/a | n/a | n/a |
| NZ_JUMH01000022.1; | | | | | |
| 1389; 1390; 16/17 | | | | | |
| 696; 896567682; | 1914 | 3136 | n/a | n/a | n/a |
| NZ_JUMH01000022.1; | | | | | |
| 1391; 1392; 16/17 | | | | | |
| 697; 387783149; | 2035 | 2752 | 4036 | n/a | n/a |
| NC_017595.1; | | | | | |
| 1393; 1394; 18/19 | | | | | |
| 698; 484021228; | 2156 | 2860 | n/a | n/a | n/a |
| NZ_KB895788.1; | | | | | |
| 1395; 1396; 21/22 | | | | | |
| 699; 269095543; | n/a | 3379 | 3997 | n/a | n/a |
| CP001819.1; | | | | | |
| 1397; 1398; 13/14 | | | | | |
| 700; 663372947; | n/a | 3087 | n/a | n/a | n/a |
| NZ_JOFL01000031.1; | | | | | |
| 1399; 1400; 32/33 | | | | | |
| 701; 692233141; | 1913 | 3135 | n/a | n/a | n/a |
| NZ_JQAK01000001.1; | | | | | |
| 1401; 1402; 24/25 | | | | | |
| 702; 692233141; | 1913 | 3135 | n/a | n/a | n/a |
| NZ_JQAK01000001.1; | | | | | |
| 1403; 1404; 24/25 | | | | | |
| 703; 896520167; | 2127 | 3504 | n/a | n/a | n/a |
| NZ_JVUI01000038.1; | | | | | |
| 1405; 1406; 16/17 | | | | | |
| 704; 194363778; | 1600 | 2699 | n/a | n/a | n/a |
| NC_011071.1; | | | | | |
| 1407; 1408; 36/37 | | | | | |
| 705; 737569369; | 1950 | 3204 | n/a | n/a | n/a |
| NZ_ARYL01000059.1; | | | | | |
| 1409; 1410; 27/28 | | | | | |
| 706; 484033611; | 1686 | 2836 | n/a | n/a | n/a |
| NZ_ANFZ01000008.1; | | | | | |
| 1411; 1412; 20/21 | | | | | |
| 707; 780834515; | n/a | 2522 | n/a | n/a | n/a |
| LADU01000087.1; | | | | | |
| 1413; 1414; 27/28 | | | | | |
| 708; 927084736; | 2268 | 3665 | 4535 | n/a | n/a |
| NZ_LITU01000056.1; | | | | | |
| 1415; 1416; 21/22 | | | | | |
| 709; 522837181; | 1406 | 2460 | 3839 | n/a | n/a |
| NZ_KE352807.1; | | | | | |
| 1417; 1418; 22/23 | | | | | |
| 710; 737569369; | 1938 | 3186 | n/a | n/a | n/a |
| NZ_ARYL01000059.1; | | | | | |
| 1419; 1420; 27/28 | | | | | |
| 711; 737577234; | 1952 | 3206 | n/a | n/a | n/a |
| NZ_AWFH01000002.1; | | | | | |
| 1421; 1422; 27/28 | | | | | |
| 712; 522837181; | 1405 | 2459 | 3838 | n/a | n/a |
| NZ_KE352807.1; | | | | | |
| 1423; 1424; 22/23 | | | | | |
| 713; 522837181; | 1505 | 2587 | 3918 | n/a | n/a |
| NZ_KE352807.1; | | | | | |
| 1425; 1426; 22/23 | | | | | |
| 714; 522837181; | 1504 | 2963 | 3918 | n/a | n/a |
| NZ_KE352807.1; | | | | | |
| 1427; 1428; 22/23 | | | | | |
| 715; 522837181; | 1410 | 2464 | 3842 | n/a | n/a |
| NZ_KE352807.1; | | | | | |
| 1429; 1430; 22/23 | | | | | |
| 716; 522837181; | n/a | 2454 | 3835 | n/a | n/a |
| NZ_KE352807.1; | | | | | |
| 1431; 1432; 22/23 | | | | | |
| 717; 522837181; | n/a | 2964 | 3918 | n/a | n/a |
| NZ_KE352807.1; | | | | | |
| 1433; 1434; 22/23 | | | | | |
| 718; 522837181; | 1763 | 2962 | 3918 | n/a | n/a |
| NZ_KE352807.1; | | | | | |
| 1435; 1436; 22/23 | | | | | |
| 719; 522837181; | 1503 | 2586 | 3918 | n/a | n/a |
| NZ_KE352807.1; | | | | | |
| 1437; 1438; 22/23 | | | | | |
| 720; 522837181; | 1372 | 2415 | 3810 | n/a | n/a |
| NZ_KE352807.1; | | | | | |
| 1439; 1440; 22/23 | | | | | |
| 721; 522837181; | n/a | 2439 | 3827 | n/a | n/a |
| NZ_KE352807.1; | | | | | |
| 1441; 1442; 22/23 | | | | | |
| 722; 822535978; | 2097 | 3462 | n/a | n/a | n/a |
| NZ_JPLE01000028.1; | | | | | |
| 1443; 1444; 35/36 | | | | | |
| 723; 924898949; | 1360 | 2395 | n/a | n/a | n/a |
| NZ_CP009452.1; | | | | | |
| 1445; 1446; 18/19 | | | | | |
| 724; 924516300; | 2252 | 3643 | n/a | n/a | n/a |
| NZ_LDVR01000003.1; | | | | | |
| 1447; 1448; 36/37 | | | | | |
| 725; 541473965; | 1413 | 2467 | 3845 | n/a | n/a |
| AWSB01000041.1; | | | | | |
| 1449; 1450; 20/21 | | | | | |
| 726; 483532492; | 1710 | n/a | n/a | n/a | n/a |
| NZ_BARE01000100.1; | | | | | |
| 1451; 1452; 19/20 | | | | | |
| 727; 655095554; | 1824 | 3224 | 4219 | n/a | n/a |
| NZ_AULE01000001.1; | | | | | |
| 1453; 1454; 22/23 | | | | | |
| 728; 541473965; | n/a | 2893 | 4376 | n/a | n/a |
| AWSB01000041.1; | | | | | |
| 1455; 1456; 20/21 | | | | | |
| 729; 545327527; | n/a | 2893 | 4376 | n/a | n/a |
| NZ_KE951412.1; | | | | | |
| 1457; 1458; 20/21 | | | | | |
| 730; 545327527; | n/a | 2893 | 4376 | n/a | n/a |
| NZ_KE951412.1; | | | | | |
| 1459; 1460; 13/14 | | | | | |
| 731; 545327527; | n/a | 2893 | 4376 | n/a | n/a |
| NZ_KE951412.1; | | | | | |
| 1461; 1462; 20/21 | | | | | |
| 732; 651445346; | n/a | 2994 | 4188 | n/a | n/a |
| NZ_AZVC01000006.1; | | | | | |
| 1463; 1464; 21/22 | | | | | |
| 733; 739650776; | 2208 | 3243 | n/a | n/a | n/a |
| NZ_KL662193.1; | | | | | |
| 1465; 1466; 29/30 | | | | | |
| 734; 260447107; | 1559 | 2651 | 3957 | n/a | n/a |
| NZ_GG703879.1; | | | | | |
| 1467; 1468; 13/14 | | | | | |
| 735; 260447107; | 1559 | 2651 | 3957 | n/a | n/a |
| NZ_GG703879.1; | | | | | |
| 1469; 1470; 20/21 | | | | | |
| 736; 260447107; | 1559 | 2651 | 3957 | n/a | n/a |
| NZ_GG703879.1; | | | | | |
| 1471; 1472; 20/21 | | | | | |
| 737; 260447107; | 1559 | 2651 | 3957 | n/a | n/a |
| NZ_GG703879.1; | | | | | |
| 1473; 1474; 20/21 | | | | | |
| 738; 260447107; | 1559 | 2651 | 3957 | n/a | n/a |
| NZ_GG703879.1; | | | | | |
| 1475; 1476; 20/21 | | | | | |
| 739; 737567115; | 1949 | 3203 | n/a | n/a | n/a |
| NZ_ARYL01000020.1; | | | | | |
| 1477; 1478; 26/27 | | | | | |
| 740; 343957487; | 1572 | 2661 | n/a | n/a | n/a |
| NZ_AEWF01000005.1; | | | | | |
| 1479; 1480; 29/30 | | | | | |
| 741; 528200987; | n/a | 3560 | 4135 | n/a | n/a |
| ATMS01000061.1; | | | | | |
| 1481; 1482; 22/23 | | | | | |
| 742; 896535166; | 1579 | 3505 | n/a | n/a | n/a |
| NZ_JVHW01000017.1; | | | | | |
| 1483; 1484; 33/34 | | | | | |
| 743; 896535166; | 2129 | 3508 | n/a | n/a | n/a |
| NZ_JVHW01000017.1; | | | | | |
| 1485; 1486; 33/34 | | | | | |
| 744; 896535166; | 1579 | 3503 | n/a | n/a | n/a |
| NZ_JVHW01000017.1; | | | | | |
| 1487; 1488; 33/34 | | | | | |
| 745; 730274767; | 2216 | 3179 | n/a | n/a | n/a |
| NZ_JSBN01000149.1; | | | | | |
| 1489; 1490; 22/23 | | | | | |
| 746; 896555871; | 1579 | 3506 | n/a | n/a | n/a |
| NZ_JVRD01000056.1; | | | | | |
| 1491; 1492; 33/34 | | | | | |
| 747; 740097110; | 1994 | 3273 | 4359 | n/a | n/a |
| NZ_JABQ01000001.1; | | | | | |
| 1493; 1494; 48/49 | | | | | |
| 748; 930169273; | 2129 | 3679 | n/a | n/a | n/a |
| NZ_LJJH01000098.1; | | | | | |
| 1495; 1496; 33/34 | | | | | |
| 749; 923067758; | 2250 | 3640 | n/a | n/a | n/a |
| NZ_CP011010.1; | | | | | |
| 1497; 1498; 33/34 | | | | | |
| 750; 484978121; | 1841 | 2866 | n/a | n/a | n/a |
| NZ_AGRB01000040.1; | | | | | |
| 1499; 1500; 33/34 | | | | | |
| 751; 664275807; | n/a | 3573 | 4280 | n/a | n/a |
| NZ_JOIX01000031.1; | | | | | |
| 1501; 1502; 39/40 | | | | | |
| 752; 737580759; | 1953 | 3207 | n/a | n/a | n/a |
| NZ_AWFH01000021.1; | | | | | |
| 1503; 1504; 31/32 | | | | | |
| 753; 484978121; | 2249 | 3639 | n/a | n/a | n/a |
| NZ_AGRB01000040.1; | | | | | |
| 1505; 1506; 33/34 | | | | | |
| 754; 896535166; | 1579 | 2667 | n/a | n/a | n/a |
| NZ_JVHW01000017.1; | | | | | |
| 1507; 1508; 33/34 | | | | | |
| 755; 896535166; | 1579 | 3395 | n/a | n/a | n/a |
| NZ_JVHW01000017.1; | | | | | |
| 1509; 1510; 33/34 | | | | | |
| 756; 434402184; | 2027 | 2766 | 4386 | n/a | n/a |
| NC_019757.1; | | | | | |
| 1511; 1512; 27/28 | | | | | |
| 757; 522837181; | n/a | 2440 | 3828 | n/a | n/a |
| NZ_KE352807.1; | | | | | |
| 1513; 1514; 22/23 | | | | | |
| 758; 640451877; | 1759 | 2959 | n/a | n/a | n/a |
| NZ_AYSW01000160.1; | | | | | |
| 1515; 1516; 13/14 | | | | | |
| 759; 640451877; | 1759 | 2959 | n/a | n/a | n/a |
| NZ_AYSW01000160.1; | | | | | |
| 1517; 1518; 17/18 | | | | | |
| 760; 640451877; | 1759 | 2959 | n/a | n/a | n/a |
| NZ_AYSW01000160.1; | | | | | |
| 1519; 1520; 16/17 | | | | | |
| 761; 528200987; | 1411 | 2465 | 3843 | n/a | n/a |
| ATMS01000061.1; | | | | | |
| 1521; 1522; 22/23 | | | | | |
| 762; 780821511; | n/a | 2521 | n/a | n/a | n/a |
| LADW01000068.1; | | | | | |
| 1523; 1524; 24/25 | | | | | |
| 763; 566231608; | 1423 | 2478 | 3854 | n/a | n/a |
| AZMH01000257.1; | | | | | |
| 1525; 1526; 19/20 | | | | | |
| 764; 736764136; | 1940 | 3188 | n/a | n/a | n/a |
| NZ_AWFD01000033.1; | | | | | |
| 1527; 1528; 27/28 | | | | | |
| 765; 737608363; | 1954 | 3208 | n/a | n/a | n/a |
| NZ_ARYJ01000002.1; | | | | | |
| 1529; 1530; 17/18 | | | | | |
| 766; 145690656; | 1322 | 2344 | n/a | n/a | n/a |
| CP000408.1; | | | | | |
| 1531; 1532; 19/20 | | | | | |
| 767; 145690656; | 1322 | 2344 | n/a | n/a | n/a |
| CP000408.1; | | | | | |
| 1533; 1534; 19/20 | | | | | |
| 768; 815863894; | n/a | 3453 | 4436 | n/a | n/a |
| NZ_LAJC01000044.1; | | | | | |
| 1535; 1536; 13/14 | | | | | |
| 769; 145690656; | 1371 | 2413 | 3808 | n/a | n/a |
| CP000408.1; | | | | | |
| 1537; 1538; 19/20 | | | | | |
| 770; 145690656; | 1371 | 2413 | 3808 | n/a | n/a |
| CP000408.1; | | | | | |
| 1539; 1540; 19/20 | | | | | |
| 771; 550281965; | 1416 | 2470 | 3848 | n/a | n/a |
| NZ_ASSJ01000070.1; | | | | | |
| 1541; 1542; 27/28 | | | | | |
| 772; 484113491; | 1731 | 2896 | n/a | n/a | n/a |
| NZ_BACX01000258.1; | | | | | |
| 1543; 1544; 10/11 | | | | | |
| 773; 145690656; | 1592 | 2949 | 3994 | n/a | n/a |
| CP000408.1; | | | | | |
| 1545; 1546; 19/20 | | | | | |
| 774; 145690656; | 1592 | 2949 | 3994 | n/a | n/a |
| CP000408.1; | | | | | |
| 1547; 1548; 19/20 | | | | | |
| 775; 483258918; | 2077 | 3422 | 4419 | n/a | n/a |
| NZ_AMFE01000033.1; | | | | | |
| 1549; 1550; 19/20 | | | | | |
| 776; 483258918; | 2077 | 3422 | 4419 | n/a | n/a |
| NZ_AMFE01000033.1; | | | | | |
| 1551; 1552; 19/20 | | | | | |
| 777; 145690656; | n/a | 2345 | n/a | n/a | n/a |
| CP000408.1; | | | | | |
| 1553; 1554; 19/20 | | | | | |
| 778; 145690656; | n/a | 2345 | n/a | n/a | n/a |
| CP000408.1; | | | | | |
| 1555; 1556; 19/20 | | | | | |
| 779; 483258918; | 2078 | 3425 | 4419 | n/a | n/a |
| NZ_AMFE01000033.1; | | | | | |
| 1557; 1558; 19/20 | | | | | |
| 780; 766595491; | 2078 | 3425 | 4419 | n/a | n/a |
| NZ_CEHM01000004.1; | | | | | |
| 1559; 1560; 19/20 | | | | | |
| 781; 737951550; | 1959 | 3562 | 4334 | n/a | n/a |
| NZ_IAAG01000075.1; | | | | | |
| 1561; 1562; 19/20 | | | | | |
| 782; 879201007; | 1483 | 2557 | 3907 | n/a | n/a |
| CKIK01000005.1; | | | | | |
| 1563; 1564; 19/20 | | | | | |
| 783; 879201007; | 1484 | 3523 | 3907 | n/a | n/a |
| CKIK01000005.1; | | | | | |
| 1565; 1566; 19/20 | | | | | |
| 784; 879201007; | 1483 | 3684 | 3907 | n/a | n/a |
| CKIK01000005.1; | | | | | |
| 1567; 1568; 19/20 | | | | | |
| 785; 879201007; | 1484 | 3524 | 3907 | n/a | n/a |
| CKIK01000005.1; 1 | | | | | |
| 569; 1570; 19/20 | | | | | |
| 786; 879201007; | 1484 | 2558 | 3907 | n/a | n/a |
| CKIK01000005.1; | | | | | |
| 1571; 1572; 19/20 | | | | | |
| 787; 483258918; | 1671 | 2812 | 4082 | n/a | n/a |
| NZ_AMFE01000033.1; | | | | | |
| 1573; 1574; 19/20 | | | | | |
| 788; 483258918; | 1671 | 2812 | 4082 | n/a | n/a |
| NZ_AMFE01000033.1; | | | | | |
| 1575; 1576; 19/20 | | | | | |
| 789; 879201007; | 1382 | 2430 | 3822 | n/a | n/a |
| CKIK01000005.1; | | | | | |
| 1577; 1578; 19/20 | | | | | |
| 790; 950938054; | 1381 | 2429 | 3821 | n/a | n/a |
| NZ_CIHL01000007.1; | | | | | |
| 1579; 1580; 19/20 | | | | | |
| 791; 739748927; | 1986 | 3254 | 4346 | n/a | n/a |
| NZ_JJMT01000011.1; | | | | | |
| 1581; 1582; 19/20 | | | | | |
| 792; 739748927; | 1986 | 3254 | 4346 | n/a | n/a |
| NZ_JJMT01000011.1; | | | | | |
| 1583; 1584; 19/20 | | | | | |
| 793; 655069822; | 1822 | 3044 | 4218 | n/a | n/a |
| NZ_KI912489.1; | | | | | |
| 1585; 1586; 19/20 | | | | | |
| 794; 655069822; | 1822 | 3044 | 4218 | n/a | n/a |
| NZ_KI912489.1; | | | | | |
| 1587; 1588; 19/20 | | | | | |
| 795; 655069822; | 1822 | 3044 | 4218 | n/a | n/a |
| NZ_KI912489.1; | | | | | |
| 1589; 1590; 19/20 | | | | | |
| 796; 655069822; | 1822 | 3044 | 4218 | n/a | n/a |
| NZ_KI912489.1; | | | | | |
| 1591; 1592; 19/20 | | | | | |
| 797; 655069822; | 1822 | 3044 | 4218 | n/a | n/a |
| NZ_KI912489.1; | | | | | |
| 1593; 1594; 19/20 | | | | | |
| 798; 655069822; | 1822 | 3044 | 4218 | n/a | n/a |
| NZ_KI912489.1; | | | | | |
| 1595; 1596; 19/20 | | | | | |
| 799; 664428976; | 1854 | 3116 | 4250 | n/a | n/a |
| NZ_KL585179.1; | | | | | |
| 1597; 1598; 21/22 | | | | | |
| 800; 325680876; | 1393 | 2444 | 3831 | n/a | n/a |
| NZ_ADKM02000123.1; | | | | | |
| 1599; 1600; 19/20 | | | | | |
| 801; 325680876; | 1507 | 3231 | 4344 | n/a | n/a |
| NZ_ADKM02000123.1; | | | | | |
| 1601; 1602; 19/20 | | | | | |
| 802; 759443001; | n/a | 3389 | 4405 | n/a | n/a |
| NZ_JDUV01000004.1; | | | | | |
| 1603; 1604; 20/21 | | | | | |
| 803; 759443001; | n/a | 3406 | 4405 | n/a | n/a |
| NZ_JDUV01000004.1; | | | | | |
| 1605; 1606; 20/21 | | | | | |
| 804; 551695014; | 1417 | 2471 | 3849 | n/a | n/a |
| AXZG01000035.1; | | | | | |
| 1607; 1608; 18/19 | | | | | |
| 805; 551695014; | 1417 | 2471 | 3849 | n/a | n/a |
| AXZG01000035.1; | | | | | |
| 1609; 1610; 9/10 | | | | | |
| 806; 818310996; | 1456 | 2526 | n/a | n/a | n/a |
| LBRK01000013.1; | | | | | |
| 1611; 1612; 29/30 | | | | | |
| 807; 213690928; | n/a | 2700 | 3992 | n/a | n/a |
| NC_011593.1; | | | | | |
| 1613; 1614; 20/21 | | | | | |
| 808; 383809261; | 1538 | 2628 | 4343 | n/a | n/a |
| NZ_AJJQ01000036.1; | | | | | |
| 1615; 1616; 18/19 | | | | | |
| 809; 383809261; | 1538 | 2628 | 4343 | n/a | n/a |
| NZ_AJJQ01000036.1; | | | | | |
| 1617; 1618; 9/10 | | | | | |
| 810; 551695014; | 1738 | 3233 | 4146 | n/a | n/a |
| AXZG01000035.1; | | | | | |
| 1619; 1620; 18/19 | | | | | |
| 811; 551695014; | 1738 | 3233 | 4146 | n/a | n/a |
| AXZG01000035.1; | | | | | |
| 1621; 1622; 9/10 | | | | | |
| 812; 484007841; | 1679 | 2823 | 4088 | n/a | n/a |
| NZ_ANAD01000138.1; | | | | | |
| 1623; 1624; 28/29 | | | | | |
| 813; 739372122; | 2204 | 3592 | 4343 | n/a | n/a |
| NZ_JQHE01000003.1; | | | | | |
| 1625; 1626; 11/12 | | | | | |
| 814; 739372122; | 2204 | 3592 | 4343 | n/a | n/a |
| NZ_JQHE01000003.1; | | | | | |
| 1627; 1628; 13/14 | | | | | |
| 815; 357386972; | 1627 | 2745 | n/a | n/a | n/a |
| NC_016109.1; | | | | | |
| 1629; 1630; 26/27 | | | | | |
| 816; 749295448; | n/a | 2965 | 4173 | n/a | n/a |
| NZ_CP006714.1; | | | | | |
| 1631; 1632; 20/21 | | | | | |
| 817; 260447107; | 1559 | 2651 | 3957 | n/a | n/a |
| NZ_GG703879.1; | | | | | |
| 1633; 1634; 20/21 | | | | | |
| 818; 260447107; | 1559 | 2651 | 3957 | n/a | n/a |
| NZ_GG703879.1; | | | | | |
| 1635; 1636; 13/14 | | | | | |
| 819; 260447107; | 1559 | 2651 | 3957 | n/a | n/a |
| NZ_GG703879.1; | | | | | |
| 1637; 1638; 20/21 | | | | | |
| 820; 260447107; | 1559 | 2651 | 3957 | n/a | n/a |
| NZ_GG703879.1; | | | | | |
| 1639; 1640; 20/21 | | | | | |
| 821; 260447107; | 1559 | 2651 | 3957 | n/a | n/a |
| NZ_GG703879.1; | | | | | |
| 1641; 1642; 20/21 | | | | | |
| 822; 749295448; | n/a | 2397 | 3797 | n/a | n/a |
| NZ_CP006714.1; | | | | | |
| 1643; 1644; 20/21 | | | | | |
| 823; 759443001; | 1442 | n/a | n/a | 2504 | n/a |
| NZ_JDUV01000004.1; | | | | | |
| 1645; 1646; 20/21 | | | | | |
| 824; 67639376; | 1460 | 2531 | n/a | n/a | n/a |
| NZ_AAHO01000116.1; | | | | | |
| 1647; 1648; 28/29 | | | | | |
| 825; 483969755; | 1703 | 2857 | n/a | n/a | n/a |
| NZ_KB891596.1; | | | | | |
| 1649; 1650; 34/35 | | | | | |
| 826; 484026206; | 1684 | 3337 | 4094 | n/a | n/a |
| NZ_ANBH01000093.1; | | | | | |
| 1651; 1652; 31/32 | | | | | |
| 827; 919546672; | n/a | 3630 | n/a | n/a | n/a |
| NZ_JOEL01000066.1; | | | | | |
| 1653; 1654; 31/32 | | | | | |
| 828; 486399859; | 2160 | 2885 | 4130 | n/a | n/a |
| NZ_KB912942.1; | | | | | |
| 1655; 1656; 24/25 | | | | | |
| 829; 815864238; | n/a | 3623 | 4437 | n/a | n/a |
| NZ_LAJC01000053.1; | | | | | |
| 1657; 1658; 22/23 | | | | | |
| 830; 879201007; | 1380 | 2427 | 3820 | n/a | n/a |
| CKIK01000005.1; | | | | | |
| 1659; 1660; 19/20 | | | | | |
| 831; 655414006; | n/a | 3053 | n/a | n/a | 4225 |
| NZ_AUBE01000007.1; | | | | | |
| 1661; 1662; 57/58 | | | | | |
| 832; 749611130; | 2225 | 3331 | n/a | n/a | n/a |
| NZ_CDHL01000044.1; | | | | | |
| 1663; 1664; 22/23 | | | | | |
| 833; 664084661; | 1849 | 3535 | 4480 | n/a | n/a |
| NZ_JOED01000001.1; | | | | | |
| 1665; 1666; 33/34 | | | | | |
| 834; 256374160; | 1650 | 2778 | n/a | n/a | n/a |
| NC_013093.1; | | | | | |
| 1667; 1668; 40/41 | | | | | |
| 835; 822214995; | n/a | 3459 | n/a | n/a | n/a |
| NZ_CP007699.1; | | | | | |
| 1669; 1670; 73/74 | | | | | |
| 836; 664084661; | 1849 | 3533 | 4479 | n/a | n/a |
| NZ_JOED01000001.1; | | | | | |
| 1671; 1672; 33/34 | | | | | |
| 837; 357386972; | 1924 | 2746 | n/a | n/a | n/a |
| NC_016109.1; | | | | | |
| 1673; 1674; 26/27 | | | | | |
| 838; 822214995; | n/a | 2387 | n/a | n/a | n/a |
| NZ_CP007699.1; | | | | | |
| 1675; 1676; 73/74 | | | | | |
| 839; 558542923; | n/a | 3128 | n/a | n/a | 4150 |
| AWQW01000003.1; | | | | | |
| 1677; 1678; 19/20 | | | | | |
| 840; 671535174; | 1909 | 3390 | n/a | n/a | n/a |
| NZ_JOHY01000024.1; | | | | | |
| 1679; 1680; 29/30 | | | | | |
| 841; 671472153; | n/a | n/a | n/a | n/a | n/a |
| NZ_JOFR01000001.1; | | | | | |
| 1681; 1682; 21/22 | | | | | |
| 842; 919546534; | n/a | 3628 | n/a | n/a | n/a |
| NZ_JOEL01000027.1; | | | | | |
| 1683; 1684; 33/34 | | | | | |
| 843; 665530468; | n/a | 3581 | n/a | n/a | n/a |
| NZ_JOCD01000052.1; | | | | | |
| 1685; 1686; 26/27 | | | | | |
| 844; 563312125; | 1420 | 2475 | n/a | n/a | n/a |
| AYTZ01000052.1; | | | | | |
| 1687; 1688; 31/32 | | | | | |
| 845; 654993549; | n/a | 3265 | n/a | n/a | n/a |
| NZ_AZVE01000016.1; | | | | | |
| 1689; 1690; 29/30 | | | | | |
| 846; 663180071; | 1987 | 3081 | n/a | n/a | n/a |
| NZ_JOBE0000043.1; | | | | | |
| 1691; 1692; 28/29 | | | | | |
| 847; 664256887; | n/a | 3578 | n/a | n/a | 4499 |
| NZ_JODF01000036.1; | | | | | |
| 1693; 1694; 51/52 | | | | | |
| 848; 558542923; | n/a | 2473 | n/a | n/a | 3851 |
| AWQW01000003.1; | | | | | |
| 1695; 1696; 19/20 | | | | | |
| 849; 906344341; | 2247 | 3515 | 4472 | n/a | n/a |
| NZ_LFXA01000009.1; | | | | | |
| 1697; 1698; 25/26 | | | | | |
| 850; 563312125; | 1440 | 2502 | n/a | n/a | n/a |
| AYTZ01000052.1; | | | | | |
| 1699; 1700; 31/32 | | | | | |
| 851; 486330103; | 1724 | 2884 | n/a | n/a | n/a |
| NZ_KB913032.1; | | | | | |
| 1701; 1702; 31/32 | | | | | |
| 852; 663693444; | n/a | 3093 | n/a | n/a | n/a |
| NZ_JOFI01000027.1; | | | | | |
| 1703; 1704; 31/32 | | | | | |
| 853; 664299296; | 2198 | 3110 | 4282 | n/a | n/a |
| NZ_JOIK01000008.1; | | | | | |
| 1705; 1706; 25/26 | | | | | |
| 854; 925610911; | 1470 | 2542 | n/a | n/a | n/a |
| LGEE01000058.1; | | | | | |
| 1707; 1708; 28/29 | | | | | |
| 855; 663317502; | 2192 | 3085 | 4500 | n/a | n/a |
| NZ_JNZ001000008.1; | | | | | |
| 1709; 1710; 40/41 | | | | | |
| 856; 384145136; | n/a | 2714 | n/a | n/a | 4004 |
| NC_017186.1; | | | | | |
| 1711; 1712; 53/54 | | | | | |
| 857; 925610911; | 2259 | 3653 | n/a | n/a | n/a |
| LGEE01000058.1; | | | | | |
| 1713; 1714; 28/29 | | | | | |
| 858; 486324513; | 1715 | 2874 | n/a | n/a | n/a |
| NZ_KB913024.1; | | | | | |
| 1715; 1716; 37/38 | | | | | |
| 859; 759802587; | n/a | 3398 | n/a | n/a | 4512 |
| NZ_CP009438.1; | | | | | |
| 1717; 1718; 50/51 | | | | | |
| 860; 921220646; | 2069 | 3636 | n/a | n/a | n/a |
| NZ_JXYI02000059.1; | | | | | |
| 1719; 1720; 27/28 | | | | | |
| 861; 818476494; | n/a | 2391 | n/a | n/a | 3793 |
| KP274854.1; | | | | | |
| 1721; 1722; 53/54 | | | | | |
| 862; 365866490; | n/a | 3547 | n/a | n/a | n/a |
| NZ_AGSW01000226.1; | | | | | |
| 1723; 1724; 28/29 | | | | | |
| 863; 365866490; | n/a | 2446 | n/a | n/a | n/a |
| NZ_AGSW01000226.1; | | | | | |
| 1725; 1726; 28/29 | | | | | |
| 864; 937182893; | 2280 | 3688 | n/a | n/a | n/a |
| NZ_LFCW01000001.1; | | | | | |
| 1727; 1728; 31/32 | | | | | |
| 865; 484022237; | 1683 | 2831 | n/a | n/a | n/a |
| NZ_ANBD01000111.1; | | | | | |
| 1729; 1730; 22/23 | | | | | |
| 866; 747653426; | n/a | 2425 | n/a | n/a | 3818 |
| CDME01000011.1; | | | | | |
| 1731; 1732; 35/36 | | | | | |
| 867; 365866490; | n/a | 3569 | n/a | n/a | n/a |
| NZ_AGSW01000226.1; | | | | | |
| 1733; 1734; 28/29 | | | | | |
| 868; 926317398; | 2258 | 3652 | n/a | n/a | n/a |
| NZ_LGDO01000015.1; | | | | | |
| 1735; 1736; 27/28 | | | | | |
| 869; 746616581; | 1351 | 2383 | n/a | n/a | n/a |
| KF954512.1; | | | | | |
| 1737; 1738; 13/14 | | | | | |
| 870; 749658562; | 2019 | 3616 | n/a | n/a | n/a |
| NZ_CP010519.1; | | | | | |
| 1739; 1740; 29/30 | | | | | |
| 871; 487404592; | n/a | 2888 | n/a | n/a | 4132 |
| NZ_ARVW01000001.1; | | | | | |
| 1741; 1742; 41/42 | | | | | |
| 872; 389759651; | 1397 | 2449 | n/a | n/a | n/a |
| NZ_AJXS01000437.1; | | | | | |
| 1743; 1744; 26/27 | | | | | |
| 873; 930491003; | n/a | 3682 | n/a | n/a | 4542 |
| NZ_LJCU01000287.1; | | | | | |
| 1745; 1746; 29/30 | | | | | |
| 874; 484016556; | 1681 | 2986 | n/a | n/a | n/a |
| NZ_ANAX01000372.1; | | | | | |
| 1747; 1748; 27/28 | | | | | |
| 875; 433601838; | n/a | 3354 | n/a | n/a | 4045 |
| NC_019673.1; | | | | | |
| 1749; 1750; 44/45 | | | | | |
| 876; 483974021; | 1705 | 3270 | n/a | n/a | n/a |
| NZ_KB891893.1; | | | | | |
| 1751; 1752; 23/24 | | | | | |
| 877; 930491003; | n/a | 2545 | n/a | n/a | 3887 |
| NZ_LJCU01000287.1; | | | | | |
| 1753; 1754; 29/30 | | | | | |
| 878; 749658562; | 1352 | 2384 | n/a | n/a | n/a |
| NZ_CP010519.1; | | | | | |
| 1755; 1756; 29/30 | | | | | |
| 879; 759755931; | 2188 | 3396 | n/a | n/a | n/a |
| NZ_JAIY01000003.1; | | | | | |
| 1757; 1758; 27/28 | | | | | |
| 880; 484007204; | 1678 | 2821 | 4086 | n/a | n/a |
| NZ_ANAC01000034.1; | | | | | |
| 1759; 1760; 25/26 | | | | | |
| 881; 433601838; | n/a | 2416 | n/a | n/a | 3811 |
| NC_019673.1; | | | | | |
| 1761; 1762; 44/45 | | | | | |
| 882; 254387191; | 1554 | 3542 | n/a | n/a | n/a |
| NZ_DS570483.1; | | | | | |
| 1763; 1764; 27/28 | | | | | |
| 883; 345007457; | 1623 | 2740 | 4024 | n/a | n/a |
| NC_015951.1; | | | | | |
| 1765; 1766; 38/39 | | | | | |
| 884; 297558985; | 2138 | 2713 | n/a | n/a | n/a |
| NC_014210.1; | | | | | |
| 1767; 1768; 27/28 | | | | | |
| 885; 927872504; | 2270 | 3457 | 4439 | n/a | n/a |
| NZ_CP011452.2; | | | | | |
| 1769; 1770; 12/13 | | | | | |
| 886; 970555001; | 2334 | 3759 | 4593 | n/a | n/a |
| NZ_LNRZ01000006.1; | | | | | |
| 1771; 1772; 25/26 | | | | | |
| 887; 960424655; | 2331 | 3754 | 4589 | n/a | n/a |
| NZ_CYUE01000025.1; | | | | | |
| 1773; 1774; 21/22 | | | | | |
| 888; 483994857; | 1723 | 2989 | 4129 | n/a | n/a |
| NZ_KB893599.1; | | | | | |
| 1775; 1776; 33/34 | | | | | |
| 889; 817524426; | 2093 | 3452 | 4435 | n/a | n/a |
| NZ_CP010429.1; | | | | | |
| 1777; 1778; 33/34 | | | | | |
| 890; 970361514; | 1481 | 2556 | 3896 | n/a | n/a |
| LOCL01000028.1; | | | | | |
| 1779; 1780; 21/22 | | | | | |
| 891; 970574347; | 2335 | 3760 | 4008 | n/a | n/a |
| NZ_LNZF01000001.1; | | | | | |
| 1781; 1782; 20/21 | | | | | |
| 892; 970574347; | 1610 | 3758 | 4373 | n/a | n/a |
| NZ_LNZF01000001.1; | | | | | |
| 1783; 1784; 20/21 | | | | | |
| 893; 961447255; | 1365 | 2402 | 3799 | n/a | n/a |
| CP013653.1; | | | | | |
| 1785; 1786; 20/21 | | | | | |
| 894; 283814236; | 1329 | 2354 | 3766 | n/a | n/a |
| CP001769.1; | | | | | |
| 1787; 1788; 35/36 | | | | | |
| 895; 746187486; | n/a | 3304 | 4506 | n/a | n/a |
| NZ_JWSY01000011.1; | | | | | |
| 1789; 1790; 12/13 | | | | | |
| 896; 960412751; | 2330 | 3753 | 4588 | n/a | n/a |
| NZ_LN881722.1; | | | | | |
| 1791; 1792; 19/20 | | | | | |
| 897; 970293907; | n/a | 2555 | n/a | n/a | n/a |
| LOHP01000076.1; | | | | | |
| 1793; 1794; 22/23 | | | | | |
| 898; 943388237; | 2295 | 3704 | 4547 | n/a | n/a |
| NZ_LIQD01000001.1; | | | | | |
| 1795; 1796; 21/22 | | | | | |
| 899; 944415035; | n/a | 3719 | n/a | n/a | 4562 |
| NZ_LIRG01000370.1; | | | | | |
| 1797; 1798; 51/52 | | | | | |
| 900; 944005810; | 2304 | 3714 | 4557 | n/a | n/a |
| NZ_LIQT01000057.1; | | | | | |
| 1799; 1800; 28/29 | | | | | |
| 901; 944020089; | n/a | 3716 | n/a | n/a | 4559 |
| NZ_LIPR01000230.1; | | | | | |
| 1801; 1802; 51/52 | | | | | |
| 902; 944020089; | n/a | 3718 | n/a | n/a | 4561 |
| NZ_LIPR01000230.1; | | | | | |
| 1803; 1804; 51/52 | | | | | |
| 903; 943922567; | n/a | 3711 | 4554 | n/a | n/a |
| NZ_LIQU01000247.1; | | | | | |
| 1805; 1806; 29/30 | | | | | |
| 904; 969919061; | 2333 | 3756 | 4591 | n/a | n/a |
| NZ_LDRR01000065.1; | | | | | |
| 1807; 1808; 21/22 | | | | | |
| 905; 969919061; | 2333 | 3756 | 4591 | n/a | n/a |
| NZ_LDRR01000065.1; | | | | | |
| 1809; 1810; 21/22 | | | | | |
| 906; 969919061; | 2333 | 3757 | 4592 | n/a | n/a |
| NZ_LDRR01000065.1; | | | | | |
| 1811; 1812; 21/22 | | | | | |
| 907; 969919061; | 2333 | 3757 | 4592 | n/a | n/a |
| NZ_LDRR01000065.1; | | | | | |
| 1813; 1814; 21/22 | | | | | |
| 908; 969919061; | 2332 | 3755 | 4590 | n/a | n/a |
| NZ_LDRR01000065.1; | | | | | |
| 1815; 1816; 21/22 | | | | | |
| 909; 969919061; | 2332 | 3755 | 4590 | n/a | n/a |
| NZ_LDRR01000065.1; | | | | | |
| 1817; 1818; 21/22 | | | | | |
| 910; 483454700; | 1722 | 2987 | 4128 | n/a | n/a |
| NZ_KB903974.1; | | | | | |
| 1819; 1820; 31/32 | | | | | |
| 911; 970579907; | 2336 | 3761 | n/a | n/a | n/a |
| NZ_KQ759763.1; | | | | | |
| 1821; 1822; 27/28 | | | | | |
| 912; 947401208; | 2311 | 3725 | n/a | n/a | n/a |
| NZ_LMKW01000010.1; | | | | | |
| 1823; 1824; 20/21 | | | | | |
| 913; 941965142; | 2293 | 3702 | n/a | n/a | n/a |
| NZ_LKIT01000002.1; | | | | | |
| 1825; 1826; 26/27 | | | | | |
| 914; 941965142; | 2293 | 3702 | n/a | n/a | n/a |
| NZ_LKIT01000002.1; | | | | | |
| 1827; 1828; 29/30 | | | | | |
| 915; 312193897; | n/a | 2720 | n/a | n/a | n/a |
| NC_014666.1; | | | | | |
| 1829; 1830; 35/36 | | | | | |
| 916; 736762362; | 1939 | 3187 | 4323 | n/a | n/a |
| NZ_CCDN010000009.1; | | | | | |
| 1831; 1832; 19/20 | | | | | |
| 917; 651596980; | 1784 | 2997 | 4190 | n/a | n/a |
| NZ_AXVB01000011.1; | | | | | |
| 1833; 1834; 19/20 | | | | | |
| 918; 850356871; | 2110 | 3482 | 4454 | n/a | n/a |
| NZ_LDWN01000016.1; | | | | | |
| 1835; 1836; 11/12 | | | | | |
| 919; 924654439; | 2253 | 3644 | 4523 | n/a | n/a |
| NZ_LIUS01000003.1; | | | | | |
| 1837; 1838; 19/20 | | | | | |
| 920; 238801497; | 1706 | 2620 | 3897 | n/a | n/a |
| NZ_CM000745.1; | | | | | |
| 1839; 1840; 19/20 | | | | | |
| 921; 651983111; | 2171 | 3001 | 4192 | n/a | n/a |
| NZ_KE387239.1; | | | | | |
| 1841; 1842; 23/24 | | | | | |
| 922; 727343482; | 1706 | 2593 | 3897 | n/a | n/a |
| NZ_JMQD01000030.1; | | | | | |
| 1843; 1844; 19/20 | | | | | |
| 923; 423557538; | 1499 | 2580 | 3913 | n/a | n/a |
| NZ_JH792114.1; | | | | | |
| 1845; 1846; 19/20 | | | | | |
| 924; 727343482; | 1706 | 3175 | 3897 | n/a | n/a |
| NZ_JMQD01000030.1; | | | | | |
| 1847; 1848; 19/20 | | | | | |
| 925; 727343482; | 1486 | 2789 | 4066 | n/a | n/a |
| NZ_JMQD01000030.1; | | | | | |
| 1849; 1850; 19/20 | | | | | |
| 926; 727343482; | 1486 | 2785 | 4066 | n/a | n/a |
| NZ_JMQD01000030.1; | | | | | |
| 1851; 1852; 19/20 | | | | | |
| 927; 727343482; | 1486 | 2786 | 4067 | n/a | n/a |
| NZ_JMQD01000030.1; | | | | | |
| 1853; 1854; 19/20 | | | | | |
| 928; 727343482; | 1762 | 2961 | 3897 | n/a | n/a |
| NZ_JMQD01000030.1; | | | | | |
| 1855; 1856; 19/20 | | | | | |
| 929; 487368297; | 1718 | 2877 | 4122 | n/a | n/a |
| NZ_KB910953.1; | | | | | |
| 1857; 1858; 19/20 | | | | | |
| 930; 423614674; | 1488 | 2562 | 3904 | n/a | n/a |
| NZ_JH792165.1; | | | | | |
| 1859; 1860; 19/20 | | | | | |
| 931; 727343482; | 1502 | 2584 | 3916 | n/a | n/a |
| NZ_JMQD01000030.1; | | | | | |
| 1861; 1862; 19/20 | | | | | |
| 932; 727343482; | 1486 | 2788 | 4066 | n/a | n/a |
| NZ_JMQD01000030.1; | | | | | |
| 1863; 1864; 19/20 | | | | | |
| 933; 727343482; | 1486 | 2583 | 3897 | n/a | n/a |
| NZ_JMQD01000030.1; | | | | | |
| 1865; 1866; 19/20 | | | | | |
| 934; 736214556; | 1935 | 3183 | 4321 | n/a | n/a |
| NZ_KN360955.1; | | | | | |
| 1867; 1868; 19/20 | | | | | |
| 935; 507060152; | 1653 | 2787 | 4068 | n/a | n/a |
| NZ_KB976714.1; | | | | | |
| 1869; 1870; 19/20 | | | | | |
| 936; 727343482; | 1486 | 2570 | 3897 | n/a | n/a |
| NZ_JMQD01000030.1; | | | | | |
| 1871; 1872; 19/20 | | | | | |
| 937; 737456981; | 1948 | 3201 | 4502 | n/a | n/a |
| NZ_KN050811.1; | | | | | |
| 1873; 1874; 11/12 | | | | | |
| 938; 880954155; | 2118 | 3491 | 4462 | n/a | n/a |
| NZ_JVPL01000109.1; | | | | | |
| 1875; 1876; 19/20 | | | | | |
| 939; 751619763; | 2026 | 3348 | 4385 | n/a | n/a |
| NZ_JXRP01000009.1; | | | | | |
| 1877; 1878; 13/14 | | | | | |
| 940; 727343482; | 1486 | 3384 | 3897 | n/a | n/a |
| NZ_JMQD01000030.1; | | | | | |
| 1879; 1880; 19/20 | | | | | |
| 941; 806951735; | 1490 | 2561 | 3905 | n/a | n/a |
| NZ_JSFD01000011.1; | | | | | |
| 1881; 1882; 19/20 | | | | | |
| 942; 736160933; | 1934 | 3182 | 4320 | n/a | n/a |
| NZ_JQMI01000015.1; | | | | | |
| 1883; 1884; 19/20 | | | | | |
| 943; 736160933; | 1934 | 3182 | 4320 | n/a | n/a |
| NZ_JQMI01000015.1; | | | | | |
| 1885; 1886; 19/20 | | | | | |
| 944; 872696015; | 2115 | 3485 | 4460 | n/a | n/a |
| NZ_LABO01000035.1; | | | | | |
| 1887; 1888; 19/20 | | | | | |
| 945; 806951735; | 1493 | 2572 | 3905 | n/a | n/a |
| NZ_JSFD01000011.1; | | | | | |
| 1889; 1890; 19/20 | | | | | |
| 946; 806951735; | 2087 | 3444 | 3905 | n/a | n/a |
| NZ_JSFD01000011.1; | | | | | |
| 1891; 1892; 19/20 | | | | | |
| 947; 950170460; | 2323 | 3742 | 4580 | n/a | n/a |
| NZ_LMTA01000046.1; | | | | | |
| 1893; 1894; 19/20 | | | | | |
| 948; 872696015; | 1498 | 2585 | 3917 | n/a | n/a |
| NZ_LAB001000035.1; | | | | | |
| 1895; 1896; 19/20 | | | | | |
| 949; 163938013; | 1596 | 2695 | 3991 | n/a | n/a |
| NC_010184.1; | | | | | |
| 1897; 1898; 13/14 | | | | | |
| 950; 872696015; | 1498 | 2782 | 4064 | n/a | n/a |
| NZ_LABO01000035.1; | | | | | |
| 1899; 1900; 19/20 | | | | | |
| 951; 238801491; | 1487 | 2560 | 3902 | n/a | n/a |
| NZ_CM000739.1; | | | | | |
| 1901; 1902; 19/20 | | | | | |
| 952; 657629081; | 1837 | 3068 | 4237 | n/a | n/a |
| NZ_AYPV01000024.1; | | | | | |
| 1903; 1904; 19/20 | | | | | |
| 953; 507035131; | 1652 | 2783 | 4065 | n/a | n/a |
| NZ_KB976800.1; | | | | | |
| 1905; 1906; 19/20 | | | | | |
| 954; 737576092; | 1951 | 3205 | 4331 | n/a | n/a |
| NZ_JRNX01000441.1; | | | | | |
| 1907; 1908; 3/4 | | | | | |
| 955; 947983982; | 2321 | 3737 | 4578 | n/a | n/a |
| NZ_LMRV01000044.1; | | | | | |
| 1909; 1910; 11/12 | | | | | |
| 956; 946400391; | 2324 | 3743 | 4581 | n/a | n/a |
| LMRY01000003.1; | | | | | |
| 1911; 1912; 23/24 | | | | | |
| 957; 423456860; | 1495 | 2568 | 3906 | n/a | n/a |
| NZ_JH791975.1; | | | | | |
| 1913; 1914; 19/20 | | | | | |
| 958; 514340871; | 1494 | 2575 | 3908 | n/a | n/a |
| NZ_KE150045.1; | | | | | |
| 1915; 1916; 19/20 | | | | | |
| 959; 946400391; | 1480 | 2554 | 3895 | n/a | n/a |
| LMRY01000003.1; | | | | | |
| 1917; 1918; 23/24 | | | | | |
| 960; 655103160; | 1825 | 3046 | 4220 | n/a | n/a |
| NZ_JMLS01000021.1; | | | | | |
| 1919; 1920; 11/12 | | | | | |
| 961; 910095435; | 1930 | 2577 | 3910 | n/a | n/a |
| NZ_JNLY01000005.1; | | | | | |
| 1921; 1922; 19/20 | | | | | |
| 962; 910095435; | 1931 | 2581 | 3910 | n/a | n/a |
| NZ_JNLY01000005.1; | | | | | |
| 1923; 1924; 19/20 | | | | | |
| 963; 910095435; | 1931 | 3519 | 4474 | n/a | n/a |
| NZ_JNLY01000005.1; | | | | | |
| 1925; 1926; 19/20 | | | | | |
| 964; 910095435; | 1930 | 3174 | 3910 | n/a | n/a |
| NZ_JNLY01000005.1; | | | | | |
| 1927; 1928; 19/20 | | | | | |
| 965; 922780240; | 2248 | 3638 | 4521 | n/a | n/a |
| NZ_LIGH01000001.1; | | | | | |
| 1929; 1930; 21/22 | | | | | |
| 966; 929005248; | 2275 | 3676 | 4539 | n/a | n/a |
| NZ_LGHP01000003.1; | | | | | |
| 1931; 1932; 21/22 | | | | | |
| 967; 767005659; | n/a | 3428 | n/a | n/a | n/a |
| NZ_CP010976.1; | | | | | |
| 1933; 1934; 19/20 | | | | | |
| 968; 507017505; | 1651 | 2780 | 4063 | n/a | n/a |
| NZ_KB976530.1; | | | | | |
| 1935; 1936; 19/20 | | | | | |
| 969; 423520617; | 1498 | 2579 | 3912 | n/a | n/a |
| NZ_JH792148.1; | | | | | |
| 1937; 1938; 19/20 | | | | | |
| 970; 910095435; | 1930 | 2574 | 4317 | n/a | n/a |
| NZ_JNLY01000005.1; | | | | | |
| 1939; 1940; 19/20 | | | | | |
| 971; 507020427; | 1497 | 2578 | 3911 | n/a | n/a |
| NZ_KB976152.1; | | | | | |
| 1941; 1942; 19/20 | | | | | |
| 972; 910095435; | 1488 | 2565 | 3900 | n/a | n/a |
| NZ_JNLY01000005.1; | | | | | |
| 1943; 1944; 19/20 | | | | | |
| 973; 483299154; | 1672 | 2813 | 4083 | n/a | n/a |
| NZ_AMGD01000001.1; | | | | | |
| 1945; 1946; 19/20 | | | | | |
| 974; 483299154; | 1672 | 2813 | 4083 | n/a | n/a |
| NZ_AMGD01000001.1; | | | | | |
| 1947; 1948; 19/20 | | | | | |
| 975; 910095435; | 1488 | 2784 | 3900 | n/a | n/a |
| NZ_JNLY01000005.1; | | | | | |
| 1949; 1950; 19/20 | | | | | |
| 976; 423468694; | 1496 | 2576 | 3909 | n/a | n/a |
| NZ_JH804628.1; | | | | | |
| 1951; 1952; 19/20 | | | | | |
| 977; 507020427; | 1491 | 2569 | 3898 | n/a | n/a |
| NZ_KB976152.1; | | | | | |
| 1953; 1954; 19/20 | | | | | |
| 978; 910095435; | 1488 | 2564 | 3900 | n/a | n/a |
| NZ_JNLY01000005.1; | | | | | |
| 1955; 1956; 19/20 | | | | | |
| 979; 910095435; | 1488 | 2566 | 3900 | n/a | n/a |
| NZ_JNLY01000005.1; | | | | | |
| 1957; 1958; 19/20 | | | | | |
| 980; 423609285; | 1501 | 2582 | 3915 | n/a | n/a |
| NZ_JH792232.1; | | | | | |
| 1959; 1960; 19/20 | | | | | |
| 981; 947966412; | 2320 | 3736 | 4576 | n/a | n/a |
| NZ_LMSD01000001.1; | | | | | |
| 1961; 1962; 19/20 | | | | | |
| 982; 947966412; | 2320 | 3736 | 4576 | n/a | n/a |
| NZ_LMSD01000001.1; | | | | | |
| 1963; 1964; 19/20 | | | | | |
| 983; 507020427; | 1497 | 2781 | 3911 | n/a | n/a |
| NZ_KB976152.1; | | | | | |
| 1965; 1966; 19/20 | | | | | |
| 984; 910095435; | 1489 | 2567 | 3899 | n/a | n/a |
| NZ_JNLY01000005.1; | | | | | |
| 1967; 1968; 19/20 | | | | | |
| 985; 950280827; | 2325 | 3744 | 4583 | n/a | n/a |
| NZ_LMSJ01000026.1; | | | | | |
| 1969; 1970; 19/20 | | | | | |
| 986; 656249802; | 1833 | 3062 | 4230 | n/a | n/a |
| NZ_AUGY01000047.1; | | | | | |
| 1971; 1972; 19/20 | | | | | |
| 987; 238801471; | 1500 | 2573 | 3914 | n/a | n/a |
| NZ_CM000719.1; | | | | | |
| 1973; 1974; 19/20 | | | | | |
| 988; 485048843; | 1711 | 2867 | 4111 | n/a | n/a |
| NZ_ALEG01000067.1; | | | | | |
| 1975; 1976; 19/20 | | | | | |
| 989; 647636934; | 1773 | 2979 | 4182 | n/a | n/a |
| NZ_JANV01000106.1; | | | | | |
| 1977; 1978; 19/20 | | | | | |
| 990; 910095435; | 1488 | 2563 | 3901 | n/a | n/a |
| NZ_JNLY01000005.1; | | | | | |
| 1979; 1980; 19/20 | | | | | |
| 991; 817541164; | 2092 | 3454 | 4438 | n/a | n/a |
| NZ_LATZ01000026.1; | | | | | |
| 1981; 1982; 19/20 | | | | | |
| 992; 488570484; | 2032 | 2770 | 4057 | n/a | n/a |
| NC_021171.1; | | | | | |
| 1983; 1984; 19/20 | | | | | |
| 993; 914730676; | 2149 | 3540 | 4481 | n/a | n/a |
| NZ_LFQJ01000032.1; | | | | | |
| 1985; 1986; 19/20 | | | | | |
| 994; 928874573; | 2052 | 3670 | 4404 | n/a | n/a |
| NZ_LIXL01000208.1; | | | | | |
| 1987; 1988; 19/20 | | | | | |
| 995; 928874573; | 2052 | 3670 | 4404 | n/a | n/a |
| NZ_LIXL01000208.1; | | | | | |
| 1989; 1990; 19/20 | | | | | |
| 996; 655165706; | 1969 | 3050 | 4222 | n/a | n/a |
| NZ_KE383843.1; | | | | | |
| 1991; 1992; 11/12 | | | | | |
| 997; 656245934; | 1832 | 3060 | 4229 | n/a | n/a |
| NZ_KE383845.1; | | | | | |
| 1993; 1994; 19/20 | | | | | |
| 998; 928874573; | 2052 | 3385 | 4404 | n/a | n/a |
| NZ_LIXL01000208.1; | | | | | |
| 1995; 1996; 19/20 | | | | | |
| 999; 928874573; | 2052 | 3385 | 4404 | n/a | n/a |
| NZ_LIXL01000208.1; | | | | | |
| 1997; 1998; 19/20 | | | | | |
| 1000; 924371245; | n/a | 3642 | n/a | n/a | n/a |
| NZ_LITP01000001.1; | | | | | |
| 1999; 2000; 19/20 | | | | | |
| 1001; 654948246; | 1819 | 3040 | 4216 | n/a | n/a |
| NZ_KI632505.1; | | | | | |
| 2001; 2002; 11/12 | | | | | |
| 1002; 657210762; | 2051 | 2750 | 4033 | n/a | n/a |
| NZ_AXZS01000018.1; | | | | | |
| 2003; 2004; 19/20 | | | | | |
| 1003; 571146044; | 1747 | 2916 | 4153 | n/a | n/a |
| BAUW01000006.1; | | | | | |
| 2005; 2006; 19/20 | | | | | |
| 1004; 935460965; | n/a | 3685 | n/a | n/a | n/a |
| NZ_LIUT01000006.1; | | | | | |
| 2007; 2008; 19/20 | | | | | |
| 1005; 651516582; | 2175 | 2995 | 4189 | n/a | n/a |
| NZ_JAEK01000001.1; | | | | | |
| 2009; 2010; 19/20 | | | | | |
| 1006; 657210762; | 1820 | 3042 | 4217 | n/a | n/a |
| NZ_AXZS01000018.1; | | | | | |
| 2011; 2012; 19/20 | | | | | |
| 1007; 657210762; | 2105 | 3476 | 4448 | n/a | n/a |
| NZ_AXZS01000018.1; | | | | | |
| 2013; 2014; 19/20 | | | | | |
| 1008; 723602665; | 1929 | 3173 | 4315 | n/a | n/a |
| NZ_JPIE01000001.1; | | | | | |
| 2015; 2016; 19/20 | | | | | |
| 1009; 657210762; | 1834 | 3065 | 4233 | n/a | n/a |
| NZ_AXZS01000018.1; | | | | | |
| 2017; 2018; 19/20 | | | | | |
| 1010; 933903534; | 1475 | 2549 | 3891 | n/a | n/a |
| LIXZ01000017.1; | | | | | |
| 2019; 2020; 11/12 | | | | | |
| 1011; 654954291; | n/a | 3041 | n/a | n/a | n/a |
| NZ_JAEO01000006.1; | | | | | |
| 2021; 2022; 19/20 | | | | | |
| 1012; 238801472; | 1482 | 2559 | 4316 | n/a | n/a |
| NZ_CM000720.1; | | | | | |
| 2023; 2024; 11/12 | | | | | |
| 1013; 651516582; | 2175 | 2995 | 4189 | n/a | n/a |
| NZ_JAEK01000001.1; | | | | | |
| 2025; 2026; 19/20 | | | | | |
| 1014; 910095435; | 1340 | 2369 | 3776 | n/a | n/a |
| NZ_JNLY01000005.1; | | | | | |
| 2027; 2028; 19/20 | | | | | |
| 1015; 403048279; | n/a | 2671 | n/a | n/a | n/a |
| NZ_HE610988.1; | | | | | |
| 2029; 2030; 19/20 | | | | | |
| 1016; 750677319; | 2222 | 3339 | 4509 | n/a | n/a |
| NZ_CBQR020000171.1; | | | | | |
| 2031; 2032; 20/21 | | | | | |
| 1017; 849078078; | 2109 | 3481 | 4453 | n/a | n/a |
| NZ_LFJO01000006.1; | | | | | |
| 2033; 2034; 18/19 | | | | | |
| 1018; 890672806; | 1712 | 3329 | 4112 | n/a | n/a |
| NZ_CP011974.1; | | | | | |
| 2035; 2036; 0/1 | | | | | |
| 1019; 890672806; | 1712 | 3446 | 4112 | n/a | n/a |
| NZ_CP011974.1; | | | | | |
| 2037; 2038; 0/1 | | | | | |
| 1020; 727078508; | n/a | 2514 | n/a | n/a | n/a |
| JRNV01000046.1; | | | | | |
| 2039; 2040; 19/20 | | | | | |
| 1021; 749299172; | 1995 | 3278 | 4363 | n/a | n/a |
| NZ_CP009241.1; | | | | | |
| 2041; 2042; 19/20 | | | | | |
| 1022; 652787974; | 2169 | 3015 | 4203 | n/a | n/a |
| NZ_AUCP01000055.1; | | | | | |
| 2043; 2044; 50/51 | | | | | |
| 1023; 652787974; | 2169 | 3015 | 4203 | n/a | n/a |
| NZ_AUCP01000055.1; | | | | | |
| 2045; 2046; 23/24 | | | | | |
| 1024; 486346141; | 1717 | 2876 | 4121 | n/a | n/a |
| NZ_KB910518.1; | | | | | |
| 2047; 2048; 19/20 | | | | | |
| 1025; 951610263; | 2328 | 3747 | 4586 | n/a | n/a |
| NZ_LMBV01000004.1; | | | | | |
| 2049; 2050; 19/20 | | | | | |
| 1026; 354585485; | n/a | 2629 | n/a | n/a | n/a |
| NZ_AGIP01000020.1; | | | | | |
| 2051; 2052; 19/20 | | | | | |
| 1027; 940346731; | 2292 | 3701 | 4546 | n/a | n/a |
| NZ_LJCO01000107.1; | | | | | |
| 2053; 2054; 19/20 | | | | | |
| 1028; 880997761; | 2119 | 3492 | 4463 | n/a | n/a |
| NZ_JVDT01000118.1; | | | | | |
| 2055; 2056; 20/21 | | | | | |
| 1029; 880997761; | 1910 | 3132 | 4300 | n/a | n/a |
| NZ_JVDT01000118.1; | | | | | |
| 2057; 2058; 20/21 | | | | | |
| 1030; 746258261; | 2038 | 3369 | 4514 | n/a | n/a |
| NZ_JUEI01000069.1; | | | | | |
| 2059; 2060; 19/20 | | | | | |
| 1031; 849059098; | 2108 | 3480 | 4452 | n/a | n/a |
| NZ_LDUE01000022.1; | | | | | |
| 2061; 2062; 22/23 | | | | | |
| 1032; 746258261; | 2003 | 3309 | 4367 | n/a | n/a |
| NZ_JUEI01000069.1; | | | | | |
| 2063; 2064; 19/20 | | | | | |
| 1033; 754884871; | 2038 | 3375 | 4513 | n/a | n/a |
| NZ_CP009282.1; | | | | | |
| 2065; 2066; 19/20 | | | | | |
| 1034; 939708105; | 2291 | 3700 | 4545 | n/a | n/a |
| NZ_LN831205.1; | | | | | |
| 2067; 2068; 19/20 | | | | | |
| 1035; 738803633; | 1970 | 3225 | 4341 | n/a | n/a |
| NZ_ASPS01000022.1; | | | | | |
| 2069; 2070; 19/20 | | | | | |
| 1036; 754841195; | 2044 | 3374 | 4398 | n/a | n/a |
| NZ_CCDG010000069.1; | | | | | |
| 2071; 2072; 19/20 | | | | | |
| 1037; 754841195; | 2016 | 3326 | 4372 | n/a | n/a |
| NZ_CCDG010000069.1; | | | | | |
| 2073; 2074; 19/20 | | | | | |
| 1038; 751586078; | 2227 | 3346 | 4384 | n/a | n/a |
| NZ_JXRR01000001.1; | | | | | |
| 2075; 2076; 19/20 | | | | | |
| 1039; 970574347; | n/a | 2749 | 4032 | n/a | n/a |
| NZ_LNZF01000001.1; | | | | | |
| 2077; 2078; 20/21 | | | | | |
| 1040; 754841195; | 2041 | 3372 | 4395 | n/a | n/a |
| NZ_CCDG010000069.1; | | | | | |
| 2079; 2080; 19/20 | | | | | |
| 1041; 927084730; | 2267 | 3664 | 4534 | n/a | n/a |
| NZ_LITU01000050.1; | | | | | |
| 2081; 2082; 20/21 | | | | | |
| 1042; 738716739; | 1965 | 3220 | 4339 | n/a | n/a |
| NZ_ASPU01000015.1; | | | | | |
| 2083; 2084; 20/21 | | | | | |
| 1043; 738716739; | 1965 | 3220 | 4339 | n/a | n/a |
| NZ_ASPU01000015.1; | | | | | |
| 2085; 2086; 20/21 | | | | | |
| 1044; 639451286; | 1756 | 2956 | 4169 | n/a | n/a |
| NZ_AWUK01000007.1; | | | | | |
| 2087; 2088; 20/21 | | | | | |
| 1045; 738803633; | 1967 | 3223 | 4340 | n/a | n/a |
| NZ_ASPS01000022.1; | | | | | |
| 2089; 2090; 19/20 | | | | | |
| 1046; 484070054; | 1688 | 2838 | 4097 | n/a | n/a |
| NZ_ANHX01000029.1; | | | | | |
| 2091; 2092; 20/21 | | | | | |
| 1047; 484070054; | 1688 | 2838 | 4097 | n/a | n/a |
| NZ_ANHX01000029.1; | | | | | |
| 2093; 2094; 20/21 | | | | | |
| 1048; 754841195; | 2043 | 3373 | 4397 | n/a | n/a |
| NZ_CCDG010000069.1; | | | | | |
| 2095; 2096; 19/20 | | | | | |
| 1049; 948045460; | 2322 | 3739 | 4579 | n/a | n/a |
| NZ_LMFO01000023.1; | | | | | |
| 2097; 2098; 22/23 | | | | | |
| 1050; 652787974; | 2169 | 3016 | 4203 | n/a | n/a |
| NZ_AUCP01000055.1; | | | | | |
| 2099; 2100; 50/51 | | | | | |
| 1051; 652787974; | 2169 | 3016 | 4203 | n/a | n/a |
| NZ_AUCP01000055.1; | | | | | |
| 2101; 2102; 23/24 | | | | | |
| 1052; 924434005; | 1459 | 2530 | 3875 | n/a | n/a |
| LIYK01000027.1; | | | | | |
| 2103; 2104; 20/21 | | | | | |
| 1053; 926268043; | 2257 | 3648 | 4524 | n/a | n/a |
| NZ_CP012600.1; | | | | | |
| 2105; 2106; 19/20 | | | | | |
| 1054; 374605177; | 2023 | 2626 | 3940 | n/a | n/a |
| NZ_AHKH01000064.1; | | | | | |
| 2107; 2108; 19/20 | | | | | |
| 1055; 392955666; | 1541 | 2630 | 3943 | n/a | n/a |
| NZ_AKKV01000020.1; | | | | | |
| 2109; 2110; 19/20 | | | | | |
| 1056; 651937013; | 1786 | 2999 | 4191 | n/a | n/a |
| NZ_JHYI01000013.1; | | | | | |
| 2111; 2112; 19/20 | | | | | |
| 1057; 843088522; | 2106 | 3478 | 4449 | n/a | n/a |
| NZ_BBIW01000001.1; | | | | | |
| 2113; 2114; 17/18 | | | | | |
| 1058; 656245934; | 1832 | 3060 | 4229 | n/a | n/a |
| NZ_KE383845.1; | | | | | |
| 2115; 2116; 19/20 | | | | | |
| 1059; 651937013; | 1786 | 2999 | 4191 | n/a | n/a |
| NZ_JHYI01000013.1; | | | | | |
| 2117; 2118; 19/20 | | | | | |
| 1060; 430748349; | 1640 | 2767 | 4055 | n/a | n/a |
| NC_019897.1; | | | | | |
| 2119; 2120; 20/21 | | | | | |
| 1061; 947983982; | 2321 | 3737 | 4578 | n/a | n/a |
| NZ_LMRV01000044.1; | | | | | |
| 2121; 2122; 11/12 | | | | | |
| 1062; 749182744; | 2015 | 3596 | 4371 | n/a | n/a |
| NZ_CP009416.1; | | | | | |
| 2123; 2124; 19/20 | | | | | |
| 1063; 802929558; | 2235 | 3059 | 4228 | n/a | n/a |
| NZ_CP009933.1; | | | | | |
| 2125; 2126; 20/21 | | | | | |
| 1064; 550916528; | 1733 | 2898 | 4138 | n/a | n/a |
| NC_022571.1; | | | | | |
| 2127; 2128; 25/26 | | | | | |
| 1065; 950938054; | 2326 | 3745 | 3907 | n/a | n/a |
| NZ_CIHL01000007.1; | | | | | |
| 2129; 2130; 19/20 | | | | | |
| 1066; 571146044; | 1431 | 2490 | 3859 | n/a | n/a |
| BAUW01000006.1; | | | | | |
| 2131; 2132; 19/20 | | | | | |
| 1067; 571146044; | 1431 | 2490 | 3859 | n/a | n/a |
| BAUW01000006.1; | | | | | |
| 2133; 2134; 19/20 | | | | | |
| 1068; 427733619; | 2221 | 2760 | 4048 | n/a | n/a |
| NC_019678.1; | | | | | |
| 2135; 2136; 22/23 | | | | | |
| 1069; 657706549; | 1838 | 3070 | n/a | n/a | n/a |
| NZ_JNLM01000001.1; | | | | | |
| 2137; 2138; 44/45 | | | | | |
| 1070; 514429123; | 1654 | 2791 | 4484 | n/a | n/a |
| NZ_KE332377.1; | | | | | |
| 2139; 2140; 29/30 | | | | | |
| 1071; 514429123; | 1654 | 2791 | 4484 | n/a | n/a |
| NZ_KE332377.1; | | | | | |
| 2141; 2142; 29/30 | | | | | |
| 1072; 514429123; | 1654 | 2791 | 4484 | n/a | n/a |
| NZ_KE332377.1; | | | | | |
| 2143; 2144; 29/30 | | | | | |
| 1073; 931536013; | 1474 | 2548 | 3890 | n/a | n/a |
| LJUL01000022.1; | | | | | |
| 2145; 2146; 38/39 | | | | | |
| 1074; 931536013; | 1474 | 2548 | 3890 | n/a | n/a |
| LJUL01000022.1; | | | | | |
| 2147; 2148; 38/39 | | | | | |
| 1075; 931536013; | 1474 | 2548 | 3890 | n/a | n/a |
| LJUL01000022.1; | | | | | |
| 2149; 2150; 38/39 | | | | | |
| 1076; 931536013; | 1474 | 2548 | 3890 | n/a | n/a |
| LJUL01000022.1; | | | | | |
| 2151; 2152; 38/39 | | | | | |
| 1077; 931536013; | 1474 | 2548 | 3890 | n/a | n/a |
| LJUL01000022.1; | | | | | |
| 2153; 2154; 38/39 | | | | | |
| 1078; 931536013; | 1474 | 2548 | 3890 | n/a | n/a |
| LJUL01000022.1; | | | | | |
| 2155; 2156; 38/39 | | | | | |
| 1079; 575082509; | 1432 | 2492 | 3860 | n/a | n/a |
| BAVS01000030.1; | | | | | |
| 2157; 2158; 19/20 | | | | | |
| 1080; 930349143; | 1362 | 2398 | 3798 | n/a | n/a |
| CP012036.1; | | | | | |
| 2159; 2160; 21/22 | | | | | |
| 1081; 575082509; | 1432 | 2492 | 3860 | n/a | n/a |
| BAVS01000030.1; | | | | | |
| 2161; 2162; 19/20 | | | | | |
| 1082; 427705465; | 1637 | 2759 | 4047 | n/a | n/a |
| NC_019676.1; | | | | | |
| 2163; 2164; 21/22 | | | | | |
| 1083; 428303693; | 1639 | 2765 | 4054 | n/a | n/a |
| NC_019753.1; | | | | | |
| 2165; 2166; 15/16 | | | | | |
| 1084; 359367134; | 1578 | 3064 | 3969 | n/a | n/a |
| NZ_AFEJ01000154.1; | | | | | |
| 2167; 2168; 21/22 | | | | | |
| 1085; 359367134; | 1578 | 3064 | 3969 | n/a | n/a |
| NZ_AFEJ01000154.1; | | | | | |
| 2169; 2170; 21/22 | | | | | |
| 1086; 325957759; | 1614 | 2726 | 4012 | n/a | n/a |
| NC_015216.1; | | | | | |
| 2171; 2172; 21/22 | | | | | |
| 1087; 851140085; | 2111 | 3601 | 4456 | n/a | n/a |
| NZ_JQKN01000008.1; | | | | | |
| 2173; 2174; 21/22 | | | | | |
| 1088; 748181452; | 2014 | 3322 | 4370 | n/a | n/a |
| NZ_JTCM01000043.1; | | | | | |
| 2175; 2176; 21/22 | | | | | |
| 1089; 748181452; | 2014 | 3322 | 4370 | n/a | n/a |
| NZ_JTCM01000043.1; | | | | | |
| 2177; 2178; 21/22 | | | | | |
| 1090; 158333233; | 1595 | 2694 | 3990 | n/a | n/a |
| NC_009925.1; | | | | | |
| 2179; 2180; 21/22 | | | | | |
| 1091; 158333233; | 1595 | 2694 | 3990 | n/a | n/a |
| NC_009925.1; | | | | | |
| 2181; 2182; 21/22 | | | | | |
| 1092; 851114167; | 2232 | 3619 | 4455 | n/a | n/a |
| NZ_LN515531.1; | | | | | |
| 2183; 2184; 23/24 | | | | | |
| 1093; 952971377; | 1379 | 2426 | 3819 | n/a | n/a |
| LN734822.1; | | | | | |
| 2185; 2186; 25/26 | | | | | |
| 1094; 428267688; | n/a | 2372 | 3779 | n/a | n/a |
| CP003653.1; | | | | | |
| 2187; 2188; 22/23 | | | | | |
| 1095; 333986242; | 1617 | 2731 | 4017 | n/a | n/a |
| NC_015574.1; | | | | | |
| 2189; 2190; 24/25 | | | | | |
| 1096; 739419616; | 2178 | 3232 | 4490 | n/a | n/a |
| NZ_KK088564.1; | | | | | |
| 2191; 2192; 20/21 | | | | | |
| 1097; 739419616; | 2178 | 3232 | 4490 | n/a | n/a |
| NZ_KK088564.1; | | | | | |
| 2193; 2194; 31/32 | | | | | |
| 1098; 427727289; | 1638 | 2763 | 4052 | n/a | n/a |
| NC_019684.1; | | | | | |
| 2195; 2196; 21/22 | | | | | |
| 1099; 890002594; | 2121 | 3496 | 4466 | n/a | n/a |
| NZ_JXCA01000005.1; | | | | | |
| 2197; 2198; 21/22 | | | | | |
| 1100; 652337551; | 1788 | 3003 | 4194 | n/a | n/a |
| NZ_KI912149.1; | | | | | |
| 2199; 2200; 31/32 | | | | | |
| 1101; 427415532; | 1535 | 2624 | 3937 | n/a | n/a |
| NZ_JH993797.1; | | | | | |
| 2201; 2202; 22/23 | | | | | |
| 1102; 551035505; | 1736 | 2901 | n/a | n/a | n/a |
| NZ_ATVS01000030.1; | | | | | |
| 2203; 2204; 20/21 | | | | | |
| 1103; 553740975; | 2172 | 2907 | 4145 | n/a | n/a |
| NZ_AWNH01000084.1; | | | | | |
| 2205; 2206; 22/23 | | | | | |
| 1104; 851351157; | 2112 | 3483 | 4457 | n/a | n/a |
| NZ_JQLY01000001.1; | | | | | |
| 2207; 2208; 25/26 | | | | | |
| 1105; 485067373; | 1713 | 2868 | 4113 | n/a | n/a |
| NZ_KB217478.1; | | | | | |
| 2209; 2210; 58/59 | | | | | |
| 1106; 451945650; | 1341 | 2373 | 3780 | n/a | n/a |
| NC_020304.1; | | | | | |
| 2211; 2212; 36/37 | | | | | |
| 1107; 938259025; | 1478 | 2552 | 3892 | n/a | n/a |
| LJSW01000006.1; | | | | | |
| 2213; 2214; 25/26 | | | | | |
| 1108; 557371823; | 1741 | 3517 | 4473 | n/a | n/a |
| NZ_ASGZ01000002.1; | | | | | |
| 2215; 2216; 26/27 | | | | | |
| 1109; 336251750; | 1619 | 2735 | 4020 | n/a | n/a |
| NC_015658.1; | | | | | |
| 2217; 2218; 26/27 | | | | | |
| 1110; 557371823; | 1418 | 2472 | 3850 | n/a | n/a |
| NZ_ASGZ01000002.1; | | | | | |
| 2219; 2220; 26/27 | | | | | |
| 1111; 484104632; | 1689 | 2839 | 4098 | n/a | n/a |
| NZ_KB235948.1; | | | | | |
| 2221; 2222; 32/33 | | | | | |
| 1112; 484104632; | 1689 | 2839 | 4098 | n/a | n/a |
| NZ_KB235948.1; | | | | | |
| 2223; 2224; 32/33 | | | | | |
| 1113; 448406329; | 1537 | 2627 | 3941 | n/a | n/a |
| NZ_AOIU01000004.1; | | | | | |
| 2225; 2226; 24/25 | | | | | |
| 1114; 751565075; | 2025 | 3345 | 4383 | n/a | n/a |
| NZ_JXCB01000004.1; | | | | | |
| 2227; 2228; 21/22 | | | | | |
| 1115; 119943794; | 2034 | 2688 | 3984 | n/a | n/a |
| NC_008709.1; | | | | | |
| 2229; 2230; 38/39 | | | | | |
| 1116; 563938926; | 2319 | 3741 | 4575 | n/a | n/a |
| NZ_AYWX01000007.1; | | | | | |
| 2231; 2232; 26/27 | | | | | |
| 1117; 451945650; | 1642 | 3367 | 4508 | n/a | n/a |
| NC_020304.1; | | | | | |
| 2233; 2234; 24/25 | | | | | |
| 1118; 563938926; | 2319 | 3735 | 4575 | n/a | n/a |
| NZ_AYWX01000007.1; | | | | | |
| 2235; 2236; 26/27 | | | | | |
| 1119; 655133038; | 1826 | 3048 | n/a | n/a | n/a |
| NZ_AUCV01000014.1; | | | | | |
| 2237; 2238; 32/33 | | | | | |
| 1120; 947704650; | 2316 | 3731 | 4572 | n/a | n/a |
| NZ_LMID01000016.1; | | | | | |
| 2239; 2240; 22/23 | | | | | |
| 1121; 294505815; | 2153 | 2710 | 4001 | n/a | n/a |
| NC_014032.1; | | | | | |
| 2241; 2242; 21/22 | | | | | |
| 1122; 294505815; | 2153 | 2710 | 4001 | n/a | n/a |
| NC_014032.1; | | | | | |
| 2243; 2244; 18/19 | | | | | |
| 1123; 947919015; | 2318 | 3734 | 4574 | n/a | n/a |
| NZ_LMHP01000012.1; | | | | | |
| 2245; 2246; 26/27 | | | | | |
| 1124; 780791108; | n/a | 2518 | 3869 | n/a | n/a |
| LADS01000058.1; | | | | | |
| 2247; 2248; 22/23 | | | | | |
| 1125; 738999090; | 2176 | 3226 | 4342 | n/a | n/a |
| NZ_KK073873.1; | | | | | |
| 2249; 2250; 26/27 | | | | | |
| 1126; 408381849; | 1519 | 2604 | 3927 | n/a | n/a |
| NZ_AMPO01000004.1; | | | | | |
| 2251; 2252; 28/29 | | | | | |
| 1127; 338209545; | n/a | 2738 | n/a | n/a | n/a |
| NC_015703.1; | | | | | |
| 2253; 2254; 33/34 | | | | | |
| 1128; 294505815; | 2153 | 2710 | 4001 | n/a | n/a |
| NC_014032.1; | | | | | |
| 2255; 2256; 19/20 | | | | | |
| 1129; 294505815; | 2153 | 2710 | 4001 | n/a | n/a |
| NC_014032.1; | | | | | |
| 2257; 2258; 18/19 | | | | | |
| 1130; 427705465; | n/a | 2370 | 3777 | n/a | n/a |
| NC_019676.1; | | | | | |
| 2259; 2260; 35/36 | | | | | |
| 1131; 427705465; | n/a | 3493 | 4046 | n/a | n/a |
| NC_019676.1; | | | | | |
| 2261; 2262; 35/36 | | | | | |
| 1132; 640169055; | 1757 | 2958 | 4487 | n/a | n/a |
| NZ_JAFS01000002.1; | | | | | |
| 2263; 2264; 40/41 | | | | | |
| 1133; 943897669; | 2298 | 3707 | 4550 | n/a | n/a |
| NZ_LIQQ01000007.1; | | | | | |
| 2265; 2266; 21/22 | | | | | |
| 1134; 943674269; | 2296 | 3705 | 4548 | n/a | n/a |
| NZ_LIQO01000205.1; | | | | | |
| 2267; 2268; 21/22 | | | | | |
| 1135; 386348020; | 1587 | 2680 | 3978 | n/a | n/a |
| NC_017584.1; | | | | | |
| 2269; 2270; 36/37 | | | | | |
| 1136; 931421682; | 1473 | 2547 | 3889 | n/a | n/a |
| LJTQ01000030.1; | | | | | |
| 2271; 2272; 29/30 | | | | | |
| 1137; 890444402; | 2122 | 3497 | 4467 | n/a | n/a |
| NZ_CP011310.1; | | | | | |
| 2273; 2274; 30/31 | | | | | |
| 1138; 41582259; | 1316 | 2337 | n/a | n/a | n/a |
| AY458641.2; | | | | | |
| 2275; 2276; 42/43 | | | | | |
| 1139; 41582259; | 2021 | 2631 | n/a | n/a | n/a |
| AY458641.2; | | | | | |
| 2277; 2278; 42/43 | | | | | |
| 1140; 554634310; | n/a | 3555 | 4147 | n/a | n/a |
| NC_022600.1; | | | | | |
| 2279; 2280; 28/29 | | | | | |
| 1141; 947721816; | 2317 | 3732 | 4573 | n/a | n/a |
| NZ_LMIB01000001.1; | | | | | |
| 2281; 2282; 22/23 | | | | | |
| 1142; 554634310; | n/a | 2377 | 3784 | n/a | n/a |
| NC_022600.1; | | | | | |
| 2283; 2284; 28/29 | | | | | |
| 1143; 483724571; | n/a | 2854 | 4106 | n/a | n/a |
| NZ_KB904821.1; | | | | | |
| 2285; 2286; 26/27 | | | | | |
| 1144; 557835508; | 1743 | 2911 | 4149 | n/a | n/a |
| NZ_AWGE01000033.1; | | | | | |
| 2287; 2288; 25/26 | | | | | |
| 1145; 575082509; | 1432 | 2492 | 3860 | n/a | n/a |
| BAVS01000030.1; | | | | | |
| 2289; 2290; 19/20 | | | | | |
| 1146; 553739852; | 1906 | 2905 | 4143 | n/a | n/a |
| NZ_AWNH01000066.1; | | | | | |
| 2291; 2292; 33/34 | | | | | |
| 1147; 484345004; | 1667 | 2806 | 4078 | n/a | n/a |
| NZ_JH947126.1; | | | | | |
| 2293; 2294; 30/31 | | | | | |
| 1148; 482909235; | n/a | 2808 | n/a | n/a | n/a |
| NZ_JH980292.1; | | | | | |
| 2295; 2296; 32/33 | | | | | |
| 1149; 737370143; | 1947 | 3200 | 4330 | n/a | n/a |
| NZ_JQKI01000040.1; | | | | | |
| 2297; 2298; 18/19 | | | | | |
| 1150; 734983081; | n/a | 3180 | n/a | n/a | n/a |
| NZ_JSXI01000073.1; | | | | | |
| 2299; 2300; 24/25 | | | | | |
| 1151; 736965849; | 1941 | 3189 | 4324 | n/a | n/a |
| NZ_JMIW01000009.1; | | | | | |
| 2301; 2302; 26/27 | | | | | |
| 1152; 483219562; | 1697 | 2849 | 4103 | n/a | n/a |
| NZ_KB901875.1; | | | | | |
| 2303; 2304; 38/39 | | | | | |
| 1153; 326793322; | 1615 | 2727 | 4013 | n/a | n/a |
| NC_015276.1; | | | | | |
| 2305; 2306; 40/41 | | | | | |
| 1154; 347753732; | 1626 | 2744 | 4027 | n/a | n/a |
| NC_016024.1; | | | | | |
| 2307; 2308; 41/42 | | | | | |
| 1155; 947472882; | 2312 | 3726 | 4566 | n/a | n/a |
| NZ_LMRH01000002.1; | | | | | |
| 2309; 2310; 21/22 | | | | | |
| 1156; 953813788; | n/a | 3748 | n/a | n/a | n/a |
| NZ_LNBE01000002.1; | | | | | |
| 2311; 2312; 12/13 | | | | | |
| 1157; 943922224; | 2301 | 3710 | 4553 | n/a | n/a |
| NZ_LIQU01000122.1; | | | | | |
| 2313; 2314; 12/13 | | | | | |
| 1158; 944029528; | 2306 | 3717 | 4560 | n/a | n/a |
| NZ_LIQZ01000126.1; | | | | | |
| 2315; 2316; 12/13 | | | | | |
| 1159; 943898694; | 2299 | 3708 | 4551 | n/a | n/a |
| NZ_LIQN01000037.1; | | | | | |
| 2317; 2318; 19/20 | | | | | |
| 1160; 953813789; | n/a | 3749 | n/a | n/a | n/a |
| NZ_LNBE01000003.1; | | | | | |
| 2319; 2320; 49/50 | | | | | |
| 1161; 943881150; | 2297 | 3706 | 4549 | n/a | n/a |
| NZ_LIPP01000138.1; | | | | | |
| 2321; 2322; 35/36 | | | | | |
| 1162; 943927948; | 2302 | 3712 | 4555 | n/a | n/a |
| NZ_LIQV01000315.1; | | | | | |
| 2323; 2324; 24/25 | | | | | |
| 1163; 943949281; | 2303 | 3713 | 4556 | n/a | n/a |
| NZ_LIPN01000124.1; | | | | | |
| 2325; 2326; 21/22 | | | | | |
| 1164; 951121600; | 2327 | 3746 | 4585 | n/a | n/a |
| NZ_LMEQ01000031.1; | | | | | |
| 2327; 2328; 21/22 | | | | | |
| 1165; 944495433; | 2307 | 3720 | 4563 | n/a | n/a |
| NZ_LIRK01000018.1; | | | | | |
| 2329; 2330; 21/22 | | | | | |
| 1166; 943899498; | 2300 | 3709 | 4552 | n/a | n/a |
| NZ_LIQN01000384.1; | | | | | |
| 2331; 2332; 21/22 | | | | | |
| 1167; 483258918; | 1392 | 2443 | 3830 | n/a | n/a |
| NZ_AMFE01000033.1; | | | | | |
| 2333; 2334; 19/20 | | | | | |
| 1168; 483258918; | 1392 | 2443 | 3830 | n/a | n/a |
| NZ_AMFE01000033.1; | | | | | |
| 2335; 2336; 19/20 | | | | | |
| 1169; 944012845; | 2305 | 3715 | 4558 | n/a | n/a |
| NZ_LIPQ01000171.1; | | | | | |
| 2337; 2338; 40/41 | | | | | |
| 1170; 664052786; | 1874 | 3097 | 4270 | n/a | n/a |
| NZ_JOES01000014.1; | | | | | |
| 2339; 2340; 21/22 | | | | | |
| 1171; 652876473; | n/a | 2634 | 3947 | n/a | n/a |
| NZ_KI912267.1; | | | | | |
| 2341; 2342; 34/35 | | | | | |
| 1172; 959926096; | 1815 | 3036 | 4337 | n/a | n/a |
| NZ_LMTZ01000085.1; | | | | | |
| 2343; 2344; 21/22 | | | | | |
| 1173; 959868240; | 2329 | 3751 | 4165 | n/a | n/a |
| NZ_CP013252.1; | | | | | |
| 2345; 2346; 18/19 | | | | | |
| 1174; 483254584; | 2157 | 2881 | 4127 | n/a | n/a |
| NZ_KB902362.1; | | | | | |
| 2347; 2348; 42/43 | | | | | |
| 1175; 655990125; | 1831 | 3600 | 4510 | n/a | n/a |
| NZ_AUBC01000024.1; | | | | | |
| 2349; 2350; 26/27 | | | | | |
| 1176; 746187665; | 2219 | 3305 | 4365 | n/a | n/a |
| NZ_JWSY01000013.1; | | | | | |
| 2351; 2352; 12/13 | | | | | |
| 1177; 443625867; | 1518 | 2603 | 4356 | n/a | n/a |
| NZ_AMLP01000127.1; | | | | | |
| 2353; 2354; 20/21 | | | | | |
| 1178; 386284588; | 1551 | 2641 | 3952 | n/a | n/a |
| NZ_AJLE01000006.1; | | | | | |
| 2355; 2356; 26/27 | | | | | |
| 1179; 826051019; | 2244 | 3631 | 4446 | n/a | n/a |
| NZ_LDES01000074.1; | | | | | |
| 2357; 2358; 22/23 | | | | | |
| 1180; 312128809; | n/a | 2718 | n/a | n/a | n/a |
| NC_014655.1; | | | | | |
| 2359; 2360; 25/26 | | | | | |
| 1181; 482849861; | 1506 | 2589 | 3920 | n/a | n/a |
| NZ_AKBU01000001.1; | | | | | |
| 2361; 2362; 3/4 | | | | | |
| 1182; 879201007; | 1380 | 2427 | 3820 | n/a | n/a |
| CKIK01000005.1; | | | | | |
| 2363; 2364; 19/20 | | | | | |
| 1183; 482849861; | 1585 | 2677 | 3963 | n/a | n/a |
| NZ_AKBU01000001.1; | | | | | |
| 2365; 2366; 3/4 | | | | | |
| 1184; 835319962; | 2213 | 3474 | 4447 | n/a | n/a |
| NZ_JTLD01000119.1; | | | | | |
| 2367; 2368; 22/23 | | | | | |
| 1185; 766607514; | 1839 | 3426 | 4421 | n/a | n/a |
| NZ_JTHO01000003.1; | | | | | |
| 2369; 2370; 20/21 | | | | | |
| 1186; 671525382; | n/a | 3130 | 4496 | n/a | n/a |
| NZ_JODL01000019.1; | | | | | |
| 2371; 2372; 31/32 | | | | | |
| 1187; 146276058; | 1591 | 2691 | 3986 | n/a | n/a |
| NC_009428.1; | | | | | |
| 2373; 2374; 32/33 | | | | | |
| 1188; 563938926; | 1620 | 2736 | 4021 | n/a | n/a |
| NZ_AYWX01000007.1; | | | | | |
| 2375; 2376; 26/27 | | | | | |
| 1189; 739662450; | n/a | n/a | n/a | n/a | n/a |
| NZ_JNFD01000038.1; | | | | | |
| 2377; 2378; 20/21 | | | | | |
| 1190; 739662450; | 1444 | n/a | n/a | n/a | n/a |
| NZ_JNFD01000038.1; | | | | | |
| 2379; 2380; 20/21 | | | | | |
| 1191; 906292938; | 1740 | 2909 | n/a | n/a | n/a |
| CXPB01000073.1; | | | | | |
| 2381; 2382; 18/19 | | | | | |
| 1192; 653556699; | 1813 | 3034 | n/a | n/a | n/a |
| NZ_AUEZ01000087.1; | | | | | |
| 2383; 2384; 26/27 | | | | | |
| 1193; 844809159; | 2107 | 3479 | 4450 | n/a | n/a |
| NZ_LDPH01000011.1; | | | | | |
| 2385; 2386; 20/21 | | | | | |
| 1194; 483961722; | n/a | 2988 | n/a | n/a | n/a |
| NZ_KB890915.1; | | | | | |
| 2387; 2388; 71/72 | | | | | |
| 1195; 739487309; | n/a | 3235 | n/a | n/a | 4504 |
| NZ_JPLW01000007.1; | | | | | |
| 2389; 2390; 27/28 | | | | | |
| 1196; 921170702; | 1884 | 3456 | n/a | n/a | n/a |
| NZ_CP009922.2; | | | | | |
| 2391; 2392; 13/14 | | | | | |
| 1197; 644043488; | 1764 | 3202 | 4174 | n/a | n/a |
| NZ_AZUQ01000001.1; | | | | | |
| 2393; 2394; 19/20 | | | | | |
| 1198; 921170702; | 1356 | 2390 | n/a | n/a | n/a |
| NZ_CP009922.2; | | | | | |
| 2395; 2396; 13/14 | | | | | |
| 1199; 254392242; | 1513 | 2598 | 3922 | n/a | n/a |
| NZ_DS570678.1; | | | | | |
| 2397; 2398; 39/40 | | | | | |
| 1200; 483975550; | 2158 | 3263 | n/a | n/a | n/a |
| NZ_KB892001.1; | | | | | |
| 2399; 2400; 30/31 | | | | | |
| 1201; 550281965; | n/a | 3336 | n/a | n/a | n/a |
| NZ_ASSJ01000070.1; | | | | | |
| 2401; 2402; 27/28 | | | | | |
| 1202; 291297538; | 1330 | 2355 | n/a | n/a | n/a |
| NC_013947.1; | | | | | |
| 2403; 2404; 29/30 | | | | | |
| 1203; 662129456; | n/a | 3532 | n/a | n/a | n/a |
| NZ_KL573544.1; | | | | | |
| 2405; 2406; 28/29 | | | | | |
| 1204; 291297538; | 1606 | 3362 | 4389 | n/a | n/a |
| NC_013947.1; | | | | | |
| 2407; 2408; 29/30 | | | | | |
| 1205; 484015294; | 1777 | 2826 | 4091 | n/a | n/a |
| NZ_ANAX01000026.1; | | | | | |
| 2409; 2410; 29/30 | | | | | |
| 1206; 655370026; | 2166 | 3051 | 4223 | n/a | n/a |
| NZ_ATZF01000001.1; | | | | | |
| 2411; 2412; 21/22 | | | | | |
| 1207; 484016825; | n/a | 2827 | n/a | n/a | n/a |
| NZ_ANAY01000003.1; | | | | | |
| 2413; 2414; 22/23 | | | | | |
| 1208; 926283036; | n/a | 3650 | n/a | n/a | n/a |
| NZ_LGEC01000103.1; | | | | | |
| 2415; 2416; 66/67 | | | | | |
| 1209; 408675720; | 1636 | 2757 | n/a | n/a | n/a |
| NC_018750.1; | | | | | |
| 2417; 2418; 27/28 | | | | | |
| 1210; 254387191; | 1554 | 3634 | n/a | n/a | n/a |
| NZ_DS570483.1; | | | | | |
| 2419; 2420; 27/28 | | | | | |
| 1211; 772744565; | n/a | 2517 | 3868 | n/a | n/a |
| NZ_JYJG01000059.1; | | | | | |
| 2421; 2422; 33/34 | | | | | |
| 1212; 919531973; | 2243 | 3627 | 4519 | n/a | n/a |
| NZ_JOEK01000003.1; | | | | | |
| 2423; 2424; 25/26 | | | | | |
| 1213; 671498318; | 2194 | 3580 | n/a | n/a | n/a |
| NZ_JOFR01000042.1; | | | | | |
| 2425; 2426; 23/24 | | | | | |
| 1214; 671498318; | 2194 | 3580 | n/a | n/a | n/a |
| NZ_JOFR01000042.1; | | | | | |
| 2427; 2428; 34/35 | | | | | |
| 1215; 514917321; | 1660 | 2796 | 4072 | n/a | n/a |
| NZ_AOPZ01000063.1; | | | | | |
| 2429; 2430; 37/38 | | | | | |
| 1216; 739097522; | 2174 | 3227 | n/a | n/a | n/a |
| NZ_KI911740.1; | | | | | |
| 2431; 2432; 28/29 | | | | | |
| 1217; 665618015; | 2187 | 3567 | 4310 | n/a | n/a |
| NZ_JODR01000032.1; | | | | | |
| 2433; 2434; 40/41 | | | | | |
| 1218; 926412094; | n/a | 3662 | n/a | n/a | 4532 |
| NZ_LGDY01000103.1; | | | | | |
| 2435; 2436; 30/31 | | | | | |
| 1219; 935540718; | n/a | 2544 | n/a | n/a | n/a |
| NZ_LGJH01000063.1; | | | | | |
| 2437; 2438; 23/24 | | | | | |
| 1220; 665536304; | 2195 | 3582 | 4297 | n/a | n/a |
| NZ_JOCD01000152.1; | | | | | |
| 2439; 2440; 35/36 | | | | | |
| 1221; 665618015; | 2187 | 3564 | 4310 | n/a | n/a |
| NZ_JODR01000032.1; | | | | | |
| 2441; 2442; 40/41 | | | | | |
| 1222; 772744565; | n/a | 3431 | 4425 | n/a | n/a |
| NZ_JYJG01000059.1; | | | | | |
| 2443; 2444; 33/34 | | | | | |
| 1223; 483112234; | 2212 | 2798 | n/a | n/a | n/a |
| NZ_AGVX02000406.1; | | | | | |
| 2445; 2446; 24/25 | | | | | |
| 1224; 739372122; | n/a | n/a | 3865 | n/a | n/a |
| NZ_JQHE01000003.1; | | | | | |
| 2447; 2448; 11/12 | | | | | |
| 1225; 739372122; | n/a | n/a | 3865 | n/a | n/a |
| NZ_JQHE01000003.1; | | | | | |
| 2449; 2450; 13/14 | | | | | |
| 1226; 664360925; | 2197 | 3114 | 4285 | n/a | n/a |
| NZ_JOGD01000054.1; | | | | | |
| 2451; 2452; 25/26 | | | | | |
| 1227; 358468594; | n/a | 2669 | n/a | n/a | n/a |
| NZ_FR873693.1; | | | | | |
| 2453; 2454; 14/15 | | | | | |
| 1228; 358468594; | n/a | 2669 | n/a | n/a | n/a |
| NZ_FR873693.1; | | | | | |
| 2455; 2456; 26/27 | | | | | |
| 1229; 358468601; | 1580 | 2670 | n/a | n/a | n/a |
| NZ_FR873700.1; | | | | | |
| 2457; 2458; 69/70 | | | | | |
| 1230; 663199697; | n/a | 3082 | n/a | n/a | n/a |
| NZ_JOHO01000012.1; | | | | | |
| 2459; 2460; 30/31 | | | | | |
| 1231; 665671804; | 2145 | 3538 | 4308 | n/a | n/a |
| NZ_JOCK01000052.1; | | | | | |
| 2461; 2462; 40/41 | | | | | |
| 1232; 254387191; | 1388 | 2436 | n/a | n/a | n/a |
| NZ_DS570483.1; | | | | | |
| 2463; 2464; 27/28 | | | | | |
| 1233; 224581098; | 1557 | 2648 | n/a | n/a | n/a |
| NZ_GG657748.1; | | | | | |
| 2465; 2466; 35/36 | | | | | |
| 1234; 110677421; | 1589 | 2685 | 3982 | n/a | n/a |
| NC_008209.1; | | | | | |
| 2467; 2468; 22/23 | | | | | |
| 1235; 563312125; | 1588 | 2682 | n/a | n/a | n/a |
| AYTZ01000052.1; | | | | | |
| 2469; 2470; 31/32 | | | | | |
| 1236; 935540718; | n/a | 3686 | n/a | n/a | n/a |
| NZ_LGJH01000063.1; | | | | | |
| 2471; 2472; 23/24 | | | | | |
| 1237; 326336949; | n/a | 2659 | n/a | n/a | n/a |
| NZ_CM001018.1; | | | | | |
| 2473; 2474; 35/36 | | | | | |
| 1238; 663670981; | n/a | 3092 | n/a | n/a | 4262 |
| NZ_JODQ01000007.1; | | | | | |
| 2475; 2476; 20/21 | | | | | |
| 1239; 546154317; | n/a | n/a | n/a | n/a | n/a |
| NZ_ACVN02000045.1; | | | | | |
| 2477; 2478; 18/19 | | | | | |
| 1240; 563312125; | 1588 | 3211 | n/a | n/a | n/a |
| AYTZ01000052.1; | | | | | |
| 2479; 2480; 31/32 | | | | | |
| 1241; 483258918; | 1392 | 2443 | 3830 | n/a | n/a |
| NZ_AMFE01000033.1; | | | | | |
| 2481; 2482; 19/20 | | | | | |
| 1242; 483258918; | 1392 | 2443 | 3830 | n/a | n/a |
| NZ_AMFE01000033.1; | | | | | |
| 2483; 2484; 19/20 | | | | | |
| 1243; 820820518; | 2237 | 3624 | n/a | n/a | n/a |
| NZ_KQ061219.1; | | | | | |
| 2485; 2486; 31/32 | | | | | |
| 1244; 514348304; | 1657 | 2795 | n/a | n/a | n/a |
| NZ_ASQH01000001.1; | | | | | |
| 2487; 2488; 26/27 | | | | | |
| 1245; 928675838; | 1386 | 2434 | n/a | n/a | n/a |
| CYTQ01000003.1; | | | | | |
| 2489; 2490; 27/28 | | | | | |
| 1246; 652698054; | 1793 | 3009 | 4198 | n/a | n/a |
| NZ_KI912610.1; | | | | | |
| 2491; 2492; 26/27 | | | | | |
| 1247; 759875025; | n/a | 3400 | n/a | n/a | n/a |
| NZ_JONS01000016.1; | | | | | |
| 2493; 2494; 12/13 | | | | | |
| 1248; 664141438; | n/a | 3584 | n/a | n/a | n/a |
| NZ_JOJM01000019.1; | | | | | |
| 2495; 2496; 29/30 | | | | | |
| 1249; 483258918; | 1392 | 2443 | 3830 | n/a | n/a |
| NZ_AMFE01000033.1; | | | | | |
| 2497; 2498; 19/20 | | | | | |
| 1250; 483258918; | 1392 | 2443 | 3830 | n/a | n/a |
| NZ_AMFE01000033.1; | | | | | |
| 2499; 2500; 19/20 | | | | | |
| 1251; 929862756; | 1732 | 2897 | 4137 | n/a | n/a |
| NZ_LGKI01000090.1; | | | | | |
| 2501; 2502; 27/28 | | | | | |
| 1252; 378759075; | 1575 | 2664 | 3966 | n/a | n/a |
| NZ_AFXE01000029.1; | | | | | |
| 2503; 2504; 22/23 | | | | | |
| 1253; 484005069; | n/a | 3551 | n/a | n/a | n/a |
| NZ_KB894416.1; | | | | | |
| 2505; 2506; 18/19 | | | | | |
| 1254; 563478461; | n/a | 2932 | 4154 | n/a | n/a |
| NZ_AYVQ01000029.1; | | | | | |
| 2507; 2508; 30/31 | | | | | |
| 1255; 482984722; | 1780 | 2848 | n/a | n/a | n/a |
| NZ_KB900605.1; | | | | | |
| 2509; 2510; 23/24 | | | | | |
| 1256; 563478461; | n/a | 2923 | 4156 | n/a | n/a |
| NZ_AYVQ01000029.1; | | | | | |
| 2511; 2512; 30/31 | | | | | |
| 1257; 563478461; | n/a | 2920 | 4156 | n/a | n/a |
| NZ_AYVQ01000029.1; | | | | | |
| 2513; 2514; 30/31 | | | | | |
| 1258; 563478461; | n/a | 2917 | 4154 | n/a | n/a |
| NZ_AYVQ01000029.1; | | | | | |
| 2515; 2516; 30/31 | | | | | |
| 1259; 563478461; | n/a | 2940 | 4161 | n/a | n/a |
| NZ_AYVQ01000029.1; | | | | | |
| 2517; 2518; 30/31 | | | | | |
| 1260; 563478461; | n/a | 2924 | 4158 | n/a | n/a |
| NZ_AYVQ01000029.1; | | | | | |
| 2519; 2520; 30/31 | | | | | |
| 1261; 563478461; | n/a | 2933 | 4154 | n/a | n/a |
| NZ_AYVQ01000029.1; | | | | | |
| 2521; 2522; 30/31 | | | | | |
| 1262; 563478461; | n/a | 2926 | 4156 | n/a | n/a |
| NZ_AYVQ01000029.1; | | | | | |
| 2523; 2524; 30/31 | | | | | |
| 1263; 563312125; | 1426 | 2482 | n/a | n/a | n/a |
| AYTZ01000052.1; | | | | | |
| 2525; 2526; 31/32 | | | | | |
| 1264; 563478461; | n/a | 2928 | 4154 | n/a | n/a |
| NZ_AYVQ01000029.1; | | | | | |
| 2527; 2528; 30/31 | | | | | |
| 1265; 652698054; | 1800 | 3014 | 4202 | n/a | n/a |
| NZ_KI912610.1; | | | | | |
| 2529; 2530; 26/27 | | | | | |
| 1266; 652698054; | 1796 | 3011 | 4200 | n/a | n/a |
| NZ_KI912610.1; | | | | | |
| 2531; 2532; 26/27 | | | | | |
| 1267; 484023389; | 2154 | 2832 | n/a | n/a | n/a |
| NZ_ANBF01000087.1; | | | | | |
| 2533; 2534; 24/25 | | | | | |
| 1268; 655569633; | 1971 | 3057 | 4491 | n/a | n/a |
| NZ_JIAI01000002.1; | | | | | |
| 2535; 2536; 32/33 | | | | | |
| 1269; 655569633; | 1971 | 3057 | 4491 | n/a | n/a |
| NZ_JIAI01000002.1; | | | | | |
| 2537; 2538; 43/44 | | | | | |
| 1270; 655569633; | 1971 | 3057 | 4491 | n/a | n/a |
| NZ_JIAI01000002.1; | | | | | |
| 2539; 2540; 32/33 | | | | | |
| 1271; 563478461; | n/a | 2925 | 4158 | n/a | n/a |
| NZ_AYVQ01000029.1; | | | | | |
| 2541; 2542; 30/31 | | | | | |
| 1272; 740292158; | 2186 | 3276 | 4361 | n/a | n/a |
| NZ_AUNB01000028.1; | | | | | |
| 2543; 2544; 22/23 | | | | | |
| 1273; 563478461; | n/a | 2921 | 4157 | n/a | n/a |
| NZ_AYVQ01000029.1; | | | | | |
| 2545; 2546; 30/31 | | | | | |
| 1274; 563478461; | n/a | 2930 | 4154 | n/a | n/a |
| NZ_AYVQ01000029.1; | | | | | |
| 2547; 2548; 30/31 | | | | | |
| 1275; 563478461; | n/a | 2927 | 4154 | n/a | n/a |
| NZ_AYVQ01000029.1; | | | | | |
| 2549; 2550; 30/31 | | | | | |
| 1276; 563478461; | n/a | 2918 | 4155 | n/a | n/a |
| NZ_AYVQ01000029.1; | | | | | |
| 2551; 2552; 30/31 | | | | | |
| 1277; 740220529; | 2185 | 3274 | 4495 | n/a | n/a |
| NZ_JHEH01000002.1; | | | | | |
| 2553; 2554; 13/14 | | | | | |
| 1278; 563478461; | n/a | 2919 | 4154 | n/a | n/a |
| NZ_AYVQ01000029.1; | | | | | |
| 2555; 2556; 30/31 | | | | | |
| 1279; 483454700; | 1722 | 2987 | 4128 | n/a | n/a |
| NZ_KB903974.1; | | | | | |
| 2557; 2558; 31/32 | | | | | |
| 1280; 835355240; | 2103 | 3475 | n/a | n/a | n/a |
| NZ_KN549147.1; | | | | | |
| 2559; 2560; 13/14 | | | | | |
| 1281; 563478461; | n/a | 2929 | 4154 | n/a | n/a |
| NZ_AYVQ01000029.1; | | | | | |
| 2561; 2562; 30/31 | | | | | |
| 1282; 563478461; | n/a | 2944 | 4158 | n/a | n/a |
| NZ_AYVQ01000029.1; | | | | | |
| 2563; 2564; 30/31 | | | | | |
| 1283; 652698054; | 1921 | 3158 | 3972 | n/a | n/a |
| NZ_KI912610.1; | | | | | |
| 2565; 2566; 26/27 | | | | | |
| 1284; 563478461; | n/a | 2931 | 4154 | n/a | n/a |
| NZ_AYVQ01000029.1; | | | | | |
| 2567; 2568; 30/31 | | | | | |
| 1285; 563478461; | n/a | 2943 | 4154 | n/a | n/a |
| NZ_AYVQ01000029.1; | | | | | |
| 2569; 2570; 30/31 | | | | | |
| 1286; 652879634; | 1802 | 3019 | 4204 | n/a | n/a |
| NZ_AZUY01000007.1; | | | | | |
| 2571; 2572; 26/27 | | | | | |
| 1287; 652698054; | 1795 | 3010 | 4199 | n/a | n/a |
| NZ_KI912610.1; | | | | | |
| 2573; 2574; 26/27 | | | | | |
| 1288; 563478461; | n/a | 2922 | 4154 | n/a | n/a |
| NZ_AYVQ01000029.1; | | | | | |
| 2575; 2576; 30/31 | | | | | |
| 1289; 652698054; | 1803 | 3020 | 4205 | n/a | n/a |
| NZ_KI912610.1; | | | | | |
| 2577; 2578; 26/27 | | | | | |
| 1290; 563478461; | n/a | 3012 | 4154 | n/a | n/a |
| NZ_AYVQ01000029.1; | | | | | |
| 2579; 2580; 30/31 | | | | | |
| 1291; 563478461; | n/a | 2945 | 4154 | n/a | n/a |
| NZ_AYVQ01000029.1; | | | | | |
| 2581; 2582; 30/31 | | | | | |
| 1292; 652698054; | 1582 | 2673 | 3972 | n/a | n/a |
| NZ_KI912610.1; | | | | | |
| 2583; 2584; 26/27 | | | | | |
| 1293; 563478461; | n/a | 2942 | 4154 | n/a | n/a |
| NZ_AYVQ01000029.1; | | | | | |
| 2585; 2586; 30/31 | | | | | |
| 1294; 652698054; | 1798 | 3013 | 4201 | n/a | n/a |
| NZ_KI912610.1; | | | | | |
| 2587; 2588; 26/27 | | | | | |
| 1295; 563938926; | 2147 | 2941 | 4162 | n/a | n/a |
| NZ_AYWX01000007.1; | | | | | |
| 2589; 2590; 26/27 | | | | | |
| 1296; 483314733; | 1699 | 2851 | n/a | n/a | n/a |
| NZ_KB902785.1; | | | | | |
| 2591; 2592; 13/14 | | | | | |
| 1297; 652698054; | 1716 | 2875 | 4120 | n/a | n/a |
| NZ_KI912610.1; | | | | | |
| 2593; 2594; 26/27 | | | | | |
| 1298; 652698054; | 1920 | 2954 | 4009 | n/a | n/a |
| NZ_KI912610.1; | | | | | |
| 2595; 2596; 26/27 | | | | | |
| 1299; 652670206; | 1791 | 3008 | 4197 | n/a | n/a |
| NZ_AUEL01000005.1; | | | | | |
| 2597; 2598; 26/27 | | | | | |
| 1300; 657698352; | 1739 | 2908 | n/a | n/a | n/a |
| NZ_JDWO01000067.1; | | | | | |
| 2599; 2600; 25/26 | | | | | |
| 1301; 653526890; | 1961 | 3033 | n/a | n/a | n/a |
| NZ_AXAZ01000002.1; | | | | | |
| 2601; 2602; 26/27 | | | | | |
| 1302; 433771415; | 1749 | 2937 | 4056 | n/a | n/a |
| NC_019973.1; | | | | | |
| 2603; 2604; 26/27 | | | | | |
| 1303; 433771415; | 1749 | 2938 | 4056 | n/a | n/a |
| NC_019973.1; | | | | | |
| 2605; 2606; 26/27 | | | | | |
| 1304; 433771415; | 1641 | 2768 | 4056 | n/a | n/a |
| NC_019973.1; | | | | | |
| 2607; 2608; 26/27 | | | | | |
| 1305; 657698352; | 1739 | 3069 | n/a | n/a | n/a |
| NZ_JDWO01000067.1; | | | | | |
| 2609; 2610; 25/26 | | | | | |
| 1306; 339501577; | 1622 | 2739 | 4023 | n/a | n/a |
| NC_015730.1; | | | | | |
| 2611; 2612; 22/23 | | | | | |
| 1307; 639168743; | 1755 | 2955 | n/a | n/a | n/a |
| NZ_AWZU01000010.1; | | | | | |
| 2613; 2614; 21/22 | | | | | |
| 1308; 433771415; | 1749 | 2935 | 4056 | n/a | n/a |
| NC_019973.1; | | | | | |
| 2615; 2616; 26/27 | | | | | |
| 1309; 484075173; | n/a | 2801 | n/a | n/a | 4076 |
| NZ_AJLK01000109.1; | | | | | |
| 2617; 2618; 27/28 | | | | | |
| 1310; 906292938; | 1384 | 2432 | n/a | n/a | n/a |
| CXPB01000073.1; | | | | | |
| 2619; 2620; 18/19 | | | | | |
| 1311; 652912253; | 1962 | 3021 | 4206 | n/a | n/a |
| NZ_ATYO01000004.1; | | | | | |
| 2621; 2622; 26/27 | | | | | |
| 1312; 906292938; | 2018 | 3332 | n/a | n/a | n/a |
| CXPB01000073.1; | | | | | |
| 2623; 2624; 18/19 | | | | | |
| 1313; 970574347; | 1768 | 2814 | 4084 | n/a | n/a |
| NZ_LNZF01000001.1; | | | | | |
| 2625; 2626; 20/21 | | | | | |
| 1314; 970574347; | 2001 | 3307 | 4074 | n/a | n/a |
| NZ_LNZF01000001.1; | | | | | |
| 2627; 2628; 20/21 | | | | | |
| 1315; 970574347; | 1768 | 3129 | 4084 | n/a | n/a |
| NZ_LNZF01000001.1; | | | | | |
| 2629; 2630; 20/21 | | | | | |

TABLE 3
Exemplary Lasso Peptidase
Lasso Peptidase Peptide No: #; Species of Origin; GI#; Accession#

1316; Uncultured marine bacterium 463 clone EBAC080-L32B05 genomic
sequence; 41582259; AY458641.2
1317; Burkholderia pseudomallei 1710b chromosome I, complete sequence;
76808520; NC_007434.1
1318; Burkholderia thailandensis E555 BTHE555_314, whole genome
shotgun sequence; 485035557; NZ_AECN01000315.1
1319; Frankia sp. CcI6 CcI6DRAFT_scaffold_51.52, whole genome shotgun
sequence; 563312125; AYTZ01000052.1
1320; Sphingopyxis alaskensis RB2256, complete genome; 103485498;
NC_008048.1
1321; Sphingopyxis alaskensis RB2256, complete genome; 103485498;
NC_008048.1
1322; Streptococcus suis SC84 complete genome, strain SC84; 253750923;
NC_012924.1
1323; Geobacter uraniireducens Rf4, complete genome; 148262085; NC_009483.1
1324; Caulobacter sp. K31, complete genome; 167643973; NC_010338.1
1325; Phenylobacterium zucineum HLK1, complete genome; 196476886;
CP000747.1
1326; Phenylobacterium zucineum HLK1, complete genome; 196476886;
CP000747.1
1327; Sanguibacter keddieii DSM 10542, complete genome; 269793358;
NC_013521.1
1328; Xylanimonas cellulosilytica DSM 15894, complete genome;
269954810; NC_013530.1
1329; Spirosoma linguale DSM 74, complete genome; 283814236;
CP001769.1
1330; Stackebrandtia nassauensis DSM 44728, complete genome; 291297538;
NC_013947.1
1331; Caulobacter segnis ATCC 21756, complete genome; 295429362;
CP002008.1
1332; Streptomyces bingchenggensis BCW-1, complete genome; 374982757;
NC_016582.1
1333; Gallionella capsiferriformans ES-2, complete genome; 302877245;
NC_014394.1
1334; Asticcacaulis excentricus CB 48 chromosome 1, complete sequence;
315497051; NC_014816.1
1335; Burkholderia gladioli BSR3 chromosome 1, complete sequence;
327367349; CP002599.1
1336; Sphingobium chlorophenolicum L-1 chromosome 1, complete
sequence; 334100279; CP002798.1
1337; Streptomyces violaceusniger Tu 4113, complete genome; 345007964;
NC_015957.1
1338; Rhodospirillum rubrum F11, complete genome; 386348020; NC_017584.1
1339; Actinoplanes sp. SE50/110, complete genome; 386845069; NC_017803.1
1340; Bacillus thuringiensis MC28, complete genome; 407703236; NC_018693.1
1341; Desulfocapsa sulfexigens DSM 10523, complete genome; 451945650;
NC_020304.1
1342; Xanthomonas citri pv. punicae str. LMG 859, whole genome shotgun
sequence; 390991205; NZ_CAGJ01000031.1
1343; Streptomyces fulvissimus DSM 40593, complete genome; 488607535;
NC_021177.1
1344; Streptomyces rapamycinicus NRRL 5491 genome; 521353217;
CP006567.1
1345; Kutzneria albida strain NRRL B-24060 contig305.1, whole genome
shotgun sequence; 662161093; NZ_JNYH01000515.1
1346; Mesorhizobium huakuii 7653R genome; 657121522; CP006581.1
1347; Mesorhizobium huakuii 7653R genome; 657121522; CP006581.1
1348; Burkholderia thailandensis E555 BTHE555_314, whole genome
shotgun sequence; 485035557; NZ_AECN01000315.1
1349; Sphingopyxis fribergensis strain Kp5.2, complete genome; 749188513;
NZ_CP009122.1
1350; Sphingopyxis fribergensis strain Kp5.2, complete genome; 749188513;
NZ_CP009122.1
1351; Streptomyces sp. ZJ306 hydroxylase, deacetylase, and hypothetical
proteins genes, complete cds; ikarugamycin gene cluster, complete sequence;
and GCN5-related N-acetyltransferase, hypothetical protein, asparagine
synthase, transcriptional regulator, ABC transporter, hypothetical proteins,
putative membrane transport protein, putative acetyltransferase, cytochrome
P450, putative alpha-glucosidase, phosphoketolase, helix-turn-helix domain-
containing protein, membrane protein, NAD-dependent epimera; 746616581;
KF954512.1
1352; Streptomyces albus strain DSM 41398, complete genome; 749658562;
NZ_CP010519.1
1353; Amycolatopsis lurida NRRL 2430, complete genome; 755908329;
CP007219.1
1354; Streptomyces lydicus A02, complete genome; 822214995; NZ_CP007699.1
1355; Streptomyces lydicus A02, complete genome; 822214995; NZ_CP007699.1
1356; Streptomyces xiamenensis strain 318, complete genome; 921170702;
NZ_CP009922.2
1357; Streptomyces sp. PBH53 genome; 852460626; CP011799.1
1358; Streptomyces sp. PBH53 genome; 852460626; CP011799.1
1359; Streptomyces sp. PBH53 genome; 852460626; CP011799.1
1360; Sphingopyxis sp. 113P3, complete genome; 924898949; NZ_CP009452.1
1361; Sphingopyxis sp. 113P3, complete genome; 924898949; NZ_CP009452.1
1362; Nostoc piscinale CENA21 genome; 930349143; CP012036.1
1363; Sphingopyxis macrogoltabida strain 203, complete genome; 938956730;
NZ_CP009429.1
1364; Sphingopyxis macrogoltabida strain 203 plasmid, complete sequence;
938956814; NZ_CP009430.1
1365; Paenibacillus sp. 32O-W, complete genome; 961447255; CP013653.1
1366; Streptomyces avermitilis MA-4680 = NBRC 14893, complete genome;
162960844; NC_003155.4
1367; Kitasatospora setae KM-6054 DNA, complete genome; 357386972;
NC_016109.1
1368; Rhodococcus jostii lariatin biosynthetic gene cluster (larA, larB, larC,
larD, larE), complete cds; 380356103; AB593691.1
1369; Rubrivivax gelatinosus IL144 DNA, complete genome; 383755859;
NC_017075.1
1370; Fischerella thennalis PCC7521 contig00099, whole genome shotgun
sequence; 484076371; NZ_AJLL01000098.1
1371; Streptococcus suis SC84 complete genome, strain SC84; 253750923;
NC_012924.1
1372; Enterococcus faecalis ATCC 29212 contig24, whole genome shotgun
sequence; 401673929; ALOD01000024.1
1373; Roseburia sp. CAG: 197 WGS project CBBL01000000 data, contig,
whole genome shotgun sequence; 524261006; CBBL010000225.1
1374; Clostridium sp. CAG: 221 WGS project CBDC01000000 data, contig,
whole genome shotgun sequence; 524362382; CBDC010000065.1
1375; Clostridium sp. CAG: 411 WGS project CBIY01000000 data, contig,
whole genome shotgun sequence; 524742306; CBIY010000075.1
1376; Novosphingobium sp. KN65.2 WGS project CCBH000000000 data,
contig SPHv1_Contig_228, whole genome shotgun sequence; 808402906;
CCBH010000144.1
1377; Mesorhizobium plurifarium genome assembly Mesorhizobium
plurifarium ORS1032T genome assembly, contig MPL1032_Contig_21,
whole genome shotgun sequence; 927916006; CCND01000014.1
1378; Kibdelosporangium sp. MJ126-NF4, whole genome shotgun sequence;
754819815; NZ_CDME01000002.1
1379; Methanobacterium formicicum genome assembly isolate Mb9,
chromosome: I; 952971377; LN734822.1
1380; Streptococcus pneumoniae strain 37, whole genome shotgun sequence;
912676034; NZ_CMPZ01000004.1
1381; Streptococcus pneumoniae strain type strain: N, whole genome shotgun
sequence; 950938054; NZ_CIHL01000007.1
1382; Streptococcus pneumoniae strain 37, whole genome shotgun sequence;
912676034; NZ_CMPZ01000004.1
1383; Klebsiella variicola genome assembly Kv4880, contig
BN1200_Contig_75, whole genome shotgun sequence; 906292938;
CXPB01000073.1
1384; Klebsiella variicola genome assembly KvT29A, contig
BN1200_Contig_98, whole genome shotgun sequence; 906304012;
CXPA01000125.1
1385; Bacillus cereus genome assembly Bacillus JRS4, contig contig000025,
whole genome shotgun sequence; 924092470; CYHM01000025.1
1386; Achromobacter sp. 2789STDY5663426 genome assembly, contig:
ERS372662SCcontig000003, whole genome shotgun sequence; 928675838;
CYTQ01000003.1
1387; Pedobacter sp. BAL39 1103467000492, whole genome shotgun
sequence; 149277373; NZ_ABCM01000005.1
1388; Streptomyces sp. Mg1 supercont1.100, whole genome shotgun
sequence; 254387191; NZ_DS570483.1
1389; Streptomyces sviceus ATCC 29083 chromosome, whole genome
shotgun sequence; 297196766; NZ_CM000951.1
1390; Streptomyces pristinaespiralis ATCC 25486 chromosome, whole
genome shotgun sequence; 297189896; NZ_CM000950.1
1391; Streptomyces roseosporus NRRL 15998 supercont3.1 genomic scaffold,
whole genome shotgun sequence; 221717172; DS999644.1
1392; Streptococcus vestibularis F0396 ctg1126932565723, whole genome
shotgun sequence; 311100538; AEKO01000007.1
1393; Ruminococcus albus 8 contig00035, whole genome shotgun sequence;
325680876; NZ_ADKM02000123.1
1394; Streptomyces sp. W007 contig00293, whole genome shotgun sequence;
365867746; NZ_AGSW01000272.1
1395; Burkholderia pseudomallei 1258a Contig0089, whole genome shotgun
sequence; 418540998; NZ_AHJB01000089.1
1396; Burkholderia pseudomallei 1258a Contig0089, whole genome shotgun
sequence; 418540998; NZ_AHJB01000089.1
1397; Rhodanobacter sp. 115 contig437, whole genome shotgun sequence;
389759651; NZ_AJXS01000437.1
1398; Rhodanobacter thiooxydans LCS2 contig057, whole genome shotgun
sequence; 389809081; NZ_AJXW01000057.1
1399; Burkholderia thailandensis MSMB43 Scaffold3, whole genome shotgun
sequence; 424903876; NZ_JH692063.1
1400; Streptomyces auratus AGR0001 Scaffold1_85, whole genome shotgun
sequence; 396995461; AJGV01000085.1
1401; Uncultured bacterium ACD_75C02634, whole genome shotgun
sequence; 406886663; AMFJ01033303.1
1402; Amycolatopsis decaplanina DSM 44594 Contig0055, whole genome
shotgun sequence; 458848256; NZ_AOHO01000055.1
1403; Streptomyces mobaraensis NBRC 13819 = DSM 40847 contig024,
whole genome shotgun sequence; 458977979; NZ_AORZ01000024.1
1404; Burkholderia mallei GB8 horse 4 contig_394, whole genome shotgun
sequence; 67639376; NZ_AAHO01000116.1
1405; Enterococcus faecalis EnGen0363 strain RMC5 acAqY-supercont1.4,
whole genome shotgun sequence; 502232520; NZ_KB944632.1
1406; Enterococcus faecalis EnGen0233 strain UAA1014 acvJV-supercont1.10.C18,
whole genome shotgun sequence; 487281881; AIZW01000018.1
1407; Pandoraea sp. SD6-2 scaffold29, whole genome shotgun sequence;
505733815; NZ_KB944444.1
1408; Streptomyces aurantiacus JA 4570 Seq28, whole genome shotgun
sequence; 514916412; NZ_AOPZ01000028.1
1409; Streptomyces aurantiacus JA 4570 Seq17, whole genome shotgun
sequence; 514916021; NZ_AOPZ01000017.1
1410; Enterococcus faecalis LA3B-2 Scaffold22, whole genome shotgun
sequence; 522837181; NZ_KE352807.1
1411; Paenibacillus alvei A6-6i-x PAAL66ix_14, whole genome shotgun
sequence; 528200987; ATMS01000061.1
1412; Dehalobacter sp. UNSWDHB Contig_139, whole genome shotgun
sequence; 544905305; NZ_AUUR01000139.1
1413; Actinobaculum sp. oral taxon 183 str. F0552 Scaffold15, whole genome
shotgun sequence; 545327527; NZ_KE951412.1
1414; Actinobaculum sp. oral taxon 183 str. F0552 A_P1HMPREF0043-1.0_Cont1.1,
whole genome shotgun sequence; 541476958; AWSB01000006.1
1415; Propionibacterium acidifaciens F0233 ctg1127964738299, whole
genome shotgun sequence; 544249812; ACVN02000045.1
1416; Rubidibacter lacunae KORDI 51-2 KR51_contig00121, whole genome
shotgun sequence; 550281965; NZ_ASSJ01000070.1
1417; Rothia aeria F0184 R_aeriaHMPREF0742-1.0_Cont136.4, whole
genome shotgun sequence; 551695014; AXZG01000035.1
1418; Candidatus Halobonum tyrrellensis G22 contig00002, whole genome
shotgun sequence; 557371823; NZ_ASGZ01000002.1
1419; Blastomonas sp. CACIA14H2 contig00049, whole genome shotgun
sequence; 563282524; AYSC01000019.1
1420; Frankia sp. CcI6 CcI6DRAFT_scaffold_51.52, whole genome shotgun
sequence; 563312125; AYTZ01000052.1
1421; Frankia sp. CeD CEDDRAFT_scaffold_22.23, whole genome shotgun
sequence; 737947180; NZ_JPGU01000023.1
1422; Clostridium butyricum DORA_1 Q607_CBUC00058, whole genome
shotgun sequence; 566226100; AZLX01000058.1
1423; Streptococcus sp. DORA_10 Q617_SPSC00257, whole genome
shotgun sequence; 566231608; AZMH01000257.1
1424; Candidatus Entotheonella gemina TSY2_contig00559, whole genome
shotgun sequence; 575423213; AZHX01000559.1
1425; Streptomyces roseosporus NRRL 15998 supercont3.1 genomic scaffold,
whole genome shotgun sequence; 221717172; DS999644.1
1426; Frankia sp. CcI6 CcI6DRAFT_scaffold_51.52, whole genome shotgun
sequence; 563312125; AYTZ01000052.1
1427; Frankia sp. Thr ThrDRAFT_scaffold_28.29, whole genome shotgun
sequence; 602262270; JENI01000029.1
1428; Novosphingobium resinovorum strain KF1 contig000008, whole
genome shotgun sequence; 738615271; NZ_JFYZ01000008.1
1429; Brevundimonas abyssalis TAR-001 DNA, contig: BAB005, whole
genome shotgun sequence; 543418148; BATC01000005.1
1430; Bacillus akibai JCM 9157, whole genome shotgun sequence;
737696658; NZ_BAUV01000025.1
1431; Bacillus boroniphilus JCM 21738 DNA, contig: contig_6, whole
genome shotgun sequence; 571146044; BAUW01000006.1
1432; Gracilibacillus boraciitolerans JCM 21714 DNA, contig: contig_30,
whole genome shotgun sequence; 575082509; BAVS01000030.1
1433; Bacterium endosymbiont of Mortierella elongata FMR23-6, whole
genome shotgun sequence; 779889750; NZ_DF850521.1
1434; Sphingopyxis sp. C-1 DNA, contig: contig_1, whole genome shotgun
sequence; 834156795; BBRO01000001.1
1435; Sphingopyxis sp. C-1 DNA, contig: contig_1, whole genome shotgun
sequence; 834156795; BBRO01000001.1
1436; Ideonella sakaiensis strain 201-F6, whole genome shotgun sequence;
928998724; NZ_BBYR01000007.1
1437; Brevundimonas sp. EAKA contig5, whole genome shotgun sequence;
737322991; NZ_JMQR01000005.1
1438; Streptomyces griseorubens strain JSD-1 contig143, whole genome
shotgun sequence; 657284919; JJMG01000143.1
1439; Frankia sp. CeD CEDDRAFT_scaffold_22.23, whole genome shotgun
sequence; 737947180; NZ_JPGU01000023.1
1440; Frankia sp. CcI6 CcI6DRAFT_scaffold_51.52, whole genome shotgun
sequence; 563312125; AYTZ01000052.1
1441; Frankia sp. CeD CEDDRAFT_scaffold_22.23, whole genome shotgun
sequence; 737947180; NZ_JPGU01000023.1
1442; Bifidobacterium callitrichos DSM 23973 contig4, whole genome
shotgun sequence; 759443001; NZ_JDUV01000004.1
1443; Streptomyces sp. JS01 contig2, whole genome shotgun sequence;
695871554; NZ_JPWW01000002.1
1444; Sphingopyxis sp. LC81 contig43, whole genome shotgun sequence;
686469310; JNFD01000038.1
1445; Sphingopyxis sp. LC81 contig24, whole genome shotgun sequence;
739659070; NZ_JNFD01000017.1
1446; Sphingopyxis sp. LC363 contig36, whole genome shotgun sequence;
739702045; NZ_JNFC01000030.1
1447; Burkholderia pseudomallei strain BEF DP42.Contig323, whole genome
shotgun sequence; 686949962; JPNR01000131.1
1448; Xanthomonas cannabis pv. phaseoli strain Nyagatare scf_52938_7,
whole genome shotgun sequence; 835885587; NZ_KN265462.1
1449; Burkholderia pseudomallei MSHR435 Y033.Contig530, whole genome
shotgun sequence; 715120018; JRFP01000024.1
1450; Candidatus Thiomargarita nelsonii isolate Hydrate Ridge contig_1164,
whole genome shotgun sequence; 723288710; JSZA01001164.1
1451; Novosphingobium sp. P6W scaffold9, whole genome shotgun sequence;
763095630; NZ_JXZE01000009.1
1452; Streptomyces griseus strain S4-7 contig113, whole genome shotgun
sequence; 764464761; NZ_JYBE01000113.1
1453; Peptococcaceae bacterium BRH_c4b BRHa_1001357, whole genome
shotgun sequence; 780813318; LADO01000010.1
1454; Streptomyces rubellomurinus subsp. indigofems strain ATCC 31304 contig-55,
whole genome shotgun sequence; 783374270; NZ_JZKG01000056.1
1455; Streptomyces sp. NRRL S-444 contig322.4, whole genome shotgun
sequence; 797049078; JZWX01001028.1
1456; Candidate division TM6 bacterium GW2011_GWF2_36_131
US03_C0013, whole genome shotgun sequence; 818310996; LBRK01000013.1
1457; Sphingobium czechense LL01 25410_1, whole genome shotgun
sequence; 861972513; JACT01000001.1
1458; Streptomyces caatingaensis strain CMAA 1322 contig02, whole genome
shotgun sequence; 906344334; NZ_LFXA01000002.1
1459; Paenibacillus polymyxa strain YUPP-8 scaffold32, whole genome
shotgun sequence; 924434005; LIYK01000027.1
1460; Burkholderia mallei GB8 horse 4 contig_394, whole genome shotgun
sequence; 67639376; NZ_AAHO01000116.1
1461; Streptomyces rimosus subsp. rimosus ATCC 10970 contig00312, whole
genome shotgun sequence; 441176881; NZ_ANSJ01000243.1
1462; Streptomyces rimosus subsp. rimosus ATCC 10970 contig00333, whole
genome shotgun sequence; 441178796; NZ_ANSJ01000259.1
1463; Streptomyces rimosus subsp. rimosus ATCC 10970 contig00312, whole
genome shotgun sequence; 441176881; NZ_ANSJ01000243.1
1464; Streptomyces rimosus subsp. rimosus ATCC 10970 contig00333, whole
genome shotgun sequence; 441178796; NZ_ANSJ01000259.1
1465; Streptomyces rimosus subsp. rimosus ATCC 10970 contig00333, whole
genome shotgun sequence; 441178796; NZ_ANSJ01000259.1
1466; Streptomyces rimosus subsp. rimosus ATCC 10970 contig00333, whole
genome shotgun sequence; 441178796; NZ_ANSJ01000259.1
1467; Streptomyces rimosus subsp. rimosus strain NRRL WC-3924 contig82.1,
whole genome shotgun sequence; 663379797; NZ_JOBW01000082.1
1468; Streptomyces sp. NRRL F-5755 P309contig7.1, whole genome shotgun
sequence; 926371541; NZ_LGCW01000295.1
1469; Streptomyces sp. NRRL F-5755 P309contig48.1, whole genome
shotgun sequence; 926371517; NZ_LGCW01000271.1
1470; Streptomyces sp. NRRL F-6491 P443contig15.1, whole genome
shotgun sequence; 925610911; LGEE01000058.1
1471; Streptomyces sp. NRRL S-444 contig322.4, whole genome shotgun
sequence; 797049078; JZWX01001028.1
1472; Actinobacteria bacterium OK074 ctg60, whole genome shotgun
sequence; 930473294; NZ_LJCV01000275.1
1473; Betaproteobacteria bacterium SG8_39 WOR_8-12_2589, whole
genome shotgun sequence; 931421682; LJTQ01000030.1
1474; Candidate division BRC1 bacterium SM23_51 WORSMTZ_10094,
whole genome shotgun sequence; 931536013; LJUL01000022.1
1475; Bacillus vietnamensis strain UCD-SED5 scaffold_15, whole genome
shotgun sequence; 933903534; LIXZ01000017.1
1476; Xanthomonas arboricola strain CITA 44 CITA_44_contig_26, whole
genome shotgun sequence; 937505789; NZ_LJGM01000026.1
1477; Xanthomonas sp. Mitacek01 contig_17, whole genome shotgun
sequence; 941965142; NZ_LKIT01000002.1
1478; Erythrobacteraceae bacterium HL-111 ITZY_scaf_51, whole genome
shotgun sequence; 938259025; LJSW01000006.1
1479; Halomonas sp. HL-93 ITZY_scaf_415, whole genome shotgun
sequence; 938285459; LJST01000237.1
1480; Paenibacillus sp. Soil724D2 contig_11, whole genome shotgun
sequence; 946400391; LMRY01000003.1
1481; Streptomyces silvensis strain ATCC 53525 53525_Assembly_Contig_22,
whole genome shotgun sequence; 970361514; LOCL01000028.1
1482; Bacillus cereus R309803 chromosome, whole genome shotgun
sequence; 238801472; NZ_CM000720.1
1483; Streptococcus pneumoniae strain PT8082 isolate E3GXY, whole
genome shotgun sequence; 935445269; NZ_CIEC02000098.1
1484; Streptococcus pneumoniae strain 37, whole genome shotgun sequence;
912676034; NZ_CMPZ01000004.1
1485; Bacillus cereus Rock3-44 chromosome, whole genome shotgun
sequence; 238801485; NZ_CM000733.1
1486; Bacillus cereus VDM006 acrHb-supercont1.1, whole genome shotgun
sequence; 507060269; NZ_KB976864.1
1487; Bacillus cereus AH1271 chromosome, whole genome shotgun
sequence; 238801491; NZ_CM000739.1
1488; Bacillus cereus VD115 supercont1.1, whole genome shotgun sequence;
423614674; NZ_JH792165.1
1489; Bacillus thuringiensis MC28, complete genome; 407703236; NC_018693.1
1490; Bacillus thuringiensis serovar andalousiensis BGSC 4AW1 chromosome,
whole genome shotgun sequence; 238801506; NZ_CM000754.1
1491; Bacillus cereus BAG3X2-1 supercont1.1, whole genome shotgun
sequence; 423416528; NZ_JH791923.1
1492; Escherichia coli strain EC2_3 Contig93, whole genome shotgun
sequence; 742921760; NZ_JWKL01000093.1
1493; Bacillus cereus NVH0597-99 gcontig2_1106483384196, whole genome
shotgun sequence; 196038187; NZ_ABDK02000003.1
1494; Bacillus cereus VD142 actaa-supercont2.2, whole genome shotgun
sequence; 514340871; NZ_KE150045.1
1495; Bacillus cereus BAG5X2-1 supercont1.1, whole genome shotgun
sequence; 423456860; NZ_JH791975.1
1496; Bacillus cereus BAG6O-2 supercont1.1, whole genome shotgun
sequence; 423468694; NZ_JH804628.1
1497; Bacillus cereus HuA2-9 acqVt-supercont1.1, whole genome shotgun
sequence; 507020427; NZ_KB976152.1
1498; Bacillus cereus HuA3-9 acqVv-supercont1.4, whole genome shotgun
sequence; 507024338; NZ_KB976146.1
1499; Bacillus cereus MC67 supercont1.2, whole genome shotgun sequence;
423557538; NZ_JH792114.1
1500; Bacillus cereus AH621 chromosome, whole genome shotgun sequence;
238801471; NZ_CM000719.1
1501; Bacillus cereus VD107 supercont1.1, whole genome shotgun sequence;
423609285; NZ_JH792232.1
1502; Bacillus cereus VDM034 supercont1.1, whole genome shotgun
sequence; 423666303; NZ_JH791809.1
1503; Enterococcus faecalis D6 supercont1.4, whole genome shotgun
sequence; 242358782; NZ_GG688629.1
1504; Enterococcus faecalis EnGen0363 strain RMC5 acAqY-supercont1.4,
whole genome shotgun sequence; 502232520; NZ_KB944632.1
1505; Enterococcus faecalis TX1341 Scfld578, whole genome shotgun
sequence; 422736691; NZ_GL457197.1
1506; Rhodobacter sphaeroides WS8N chromosome chrI, whole genome
shotgun sequence; 332561612; NZ_CM001161.1
1507; Ruminococcus albus 8 contig00035, whole genome shotgun sequence;
325680876; NZ_ADKM02000123.1
1508; Brevundimonas diminuta ATCC 11568 BDIM_scaffold00005, whole
genome shotgun sequence; 329889017; NZ_GL883086.1
1509; Brevundimonas diminuta 470-4 Scfld7, whole genome shotgun
sequence; 444405902; NZ_KB291784.1
1510; Clostridium butyricum 5521 gcontig_1106103650482, whole genome
shotgun sequence; 182420360; NZ_ABDT01000120.2
1511; Clostridium butyricum strain HM-68 Contig83, whole genome shotgun
sequence; 760273878; NZ_JXBT01000001.1
1512; Xanthomonas citri pv. punicae str. LMG 859, whole genome shotgun
sequence; 390991205; NZ_CAGJ01000031.1
1513; Streptomyces clavuligerus ATCC 27064 supercont1.55, whole genome
shotgun sequence; 254392242; NZ_DS570678.1
1514; Streptomyces rimosus subsp. rimosus ATCC 10970 contig00312, whole
genome shotgun sequence; 441176881; NZ_ANSJ01000243.1
1515; Streptomyces rimosus subsp. rimosus ATCC 10970 contig00333, whole
genome shotgun sequence; 441178796; NZ_ANSJ01000259.1
1516; Streptomyces viridochromogenes DSM 40736 supercont1.1, whole
genome shotgun sequence; 224581107; NZ_GG657757.1
1517; Streptomyces viridochromogenes DSM 40736 supercont1.1, whole
genome shotgun sequence; 224581107; NZ_GG657757.1
1518; Streptomyces viridochromogenes Tue57 Seq127, whole genome
shotgun sequence; 443625867; NZ_AMLP01000127.1
1519; Methanobacterium formicicum DSM 3637 Contig04, whole genome
shotgun sequence; 408381849; NZ_AMPO01000004.1
1520; Burkholderia mallei GB8 horse 4 contig_394, whole genome shotgun
sequence; 67639376; NZ_AAHO01000116.1
1521; Sphingobium yanoikuyae ATCC 51230 supercont1.1, whole genome
shotgun sequence; 427407324; NZ_JH992904.1
1522; Sphingobium yanoikuyae strain SHJ scaffold2, whole genome shotgun
sequence; 893711333; NZ_KQ235984.1
1523; Burkholderia mallei GB8 horse 4 contig_394, whole genome shotgun
sequence; 67639376; NZ_AAHO01000116.1
1524; Burkholderia pseudomallei 1710b chromosome I, complete sequence;
76808520; NC_007434.1
1525; Burkholderia pseudomallei 1258a Contig0089, whole genome shotgun
sequence; 418540998; NZ_AHJB01000089.1
1526; Burkholderia pseudomallei strain BEF DP42.Contig323, whole genome
shotgun sequence; 686949962; JPNR01000131.1
1527; [Eubacterium] cellulosolvens 6 chromosome, whole genome shotgun
sequence; 389575461; NZ_CM001487.1
1528; Streptomyces mobaraensis NBRC 13819 = DSM 40847 contig024,
whole genome shotgun sequence; 458977979; NZ_AORZ01000024.1
1529; Streptomyces mobaraensis NBRC 13819 = DSM 40847 contig079,
whole genome shotgun sequence; 458984960; NZ_AORZ01000079.1
1530; Amycolatopsis azurea DSM 43854 contig60, whole genome shotgun
sequence; 451338568; NZ_ANMG01000060.1
1531; Streptomyces pristinaespiralis ATCC 25486 chromosome, whole
genome shotgun sequence; 297189896; NZ_CM000950.1
1532; Xanthomonas axonopodis pv. malvacearum str. GSPB1386 1386_Scaffold6,
whole genome shotgun sequence; 418516056; NZ_AHIB01000006.1
1533; Burkholderia thailandensis MSMB43 Scaffold3, whole genome shotgun
sequence; 424903876; NZ_JH692063.1
1534; Xanthomonas gardneri ATCC 19865 XANTHO7DRAF_Contig52,
whole genome shotgun sequence; 325923334; NZ_AEQX01000392.1
1535; Leptolyngbya sp. PCC 7375 Lepto7375DRAFT_LPA.5, whole genome
shotgun sequence; 427415532; NZ_JH993797.1
1536; Streptomyces auratus AGR0001 Scaffold1, whole genome shotgun
sequence; 398790069; NZ_JH725387.1
1537; Halosimplex carlsbadense 2-9-1 contig_4, whole genome shotgun
sequence; 448406329; NZ_AOIU01000004.1
1538; Rothia aeria F0474 contig00003, whole genome shotgun sequence;
383809261; NZ_AJJQ01000036.1
1539; Sphingobium japonicum BiD32, whole genome shotgun sequence;
494022722; NZ_CAVK010000217.1
1540; Amycolatopsis decaplanina DSM 44594 Contig0055, whole genome
shotgun sequence; 458848256; NZ_AOHO01000055.1
1541; Fictibacillus macauensis ZFHKF-1 Contig20, whole genome shotgun
sequence; 392955666; NZ_AKKV01000020.1
1542; Paenibacillus sp. Aloe-11 GW8_15, whole genome shotgun sequence;
375307420; NZ_JH601049.1
1543; Rhodanobacter denitrificans strain 116-2 contig032, whole genome
shotgun sequence; 389798210; NZ_AJXV01000032.1
1544; Caulobacter sp. AP07 PMI01_contig_53.53, whole genome shotgun
sequence; 399069941; NZ_AKKF01000033.1
1545; Novosphingobium sp. AP12 PMI02_contig_78.78, whole genome
shotgun sequence; 399058618; NZ_AKKE01000021.1
1546; Sphingobium sp. AP49 PMI04_contig490.490, whole genome shotgun
sequence; 398386476; NZ_AJVL01000086.1
1547; Moorea producens 3L scf52054, whole genome shotgun sequence;
332710503; NZ_GL890955.1
1548; Rhodanobacter sp. 115 contig437, whole genome shotgun sequence;
389759651; NZ_AJXS01000437.1
1549; Pedobacter sp. BAL39 1103467000500, whole genome shotgun
sequence; 149277003; NZ_ABCM01000004.1
1550; Pedobacter sp. BAL39 1103467000492, whole genome shotgun
sequence; 149277373; NZ_ABCM01000005.1
1551; Sulfurovum sp. AR contig00449, whole genome shotgun sequence;
386284588; NZ_AJLE01000006.1
1552; Mucilaginibacter paludis DSM 18603 chromosome, whole genome
shotgun sequence; 373951708; NZ_CM001403.1
1553; Magnetospirillum caucaseum strain SO-1 contig00006, whole genome
shotgun sequence; 458904467; NZ_AONQ01000006.1
1554; Streptomyces sp. Mg1 supercont1.100, whole genome shotgun
sequence; 254387191; NZ_DS570483.1
1555; Sphingomonas sp. LH128 Contig3, whole genome shotgun sequence;
402821166; NZ_ALVC01000003.1
1556; Sphingomonas sp. LH128 Contig8, whole genome shotgun sequence;
402821307; NZ_ALVC01000008.1
1557; Streptomyces sp. AA4 supercont1.3, whole genome shotgun sequence;
224581098; NZ_GG657748.1
1558; Cecembia lonarensis LW9 contig000133, whole genome shotgun
sequence; 406663945; NZ_AMGM01000133.1
1559; Actinomyces sp. oral taxon 848 str. F0332 Scfld0, whole genome
shotgun sequence; 260447107; NZ_GG703879.1
1560; Streptomyces ipomoeae 91-03 gcontig_1108499715961, whole genome
shotgun sequence; 429196334; NZ_AEJC01000180.1
1561; Frankia sp. QA3 chromosome, whole genome shotgun sequence;
392941286; NZ_CM001489.1
1562; Fischerella thermalis PCC 7521 contig00099, whole genome shotgun
sequence; 484076371; NZ_AJLL01000098.1
1563; Rhodobacter sp. AKP1 contig19, whole genome shotgun sequence;
429208285; NZ_ANFS01000019.1
1564; Rubrivivax benzoatilyticus JA2 = ATCC BAA-35 strain JA2 contig_155,
whole genome shotgun sequence; 332527785; NZ_AEWG01000155.1
1565; Burkholderia thailandensis E555 BTHE555_314, whole genome
shotgun sequence; 485035557; NZ_AECN01000315.1
1566; Burkholderia thailandensis E555 BTHE555_314, whole genome
shotgun sequence; 485035557; NZ_AECN01000315.1
1567; Streptomyces chartreusis NRRL 12338 12338_Doro1_scaffold19,
whole genome shotgun sequence; 381200190; NZ_JH164855.1
1568; Streptomyces globisporus C-1027 Scaffold24_1, whole genome shotgun
sequence; 410651191 ; NZ_AJUO01000171.1
1569; Streptomyces roseosporus NRRL 15998 supercont3.1 genomic scaffold,
whole genome shotgun sequence; 221717172; DS999644.1
1570; Burkholderia oklahomensis EO147 PMP6xxBPSxxE0147-248, whole
genome shotgun sequence; 149146238; NZ_ABBF01000248.1
1571; Burkholderia oklahomensis C6786 PMP6xxBOKxxC6786-168, whole
genome shotgun sequence; 149147045; NZ_ABBG01000168.1
1572; Candidatus Odyssella thessalonicensis L13 HMO_scaffold00016, whole
genome shotgun sequence; 343957487; NZ_AEWF01000005.1
1573; Candidatus Odyssella thessalonicensis L13 HMO_scaffold00016, whole
genome shotgun sequence; 343957487; NZ_AEWF01000005.1
1574; Sphingobium yanoikuyae XLDN2-5 contig000022, whole genome
shotgun sequence; 378759068; NZ_AFXE01000022.1
1575; Sphingobium yanoikuyae XLDN2-5 contig000029, whole genome
shotgun sequence; 378759075; NZ_AFXE01000029.1
1576; Paenibacillus peoriae KCTC 3763 contig9, whole genome shotgun
sequence; 389822526; NZ_AGFX01000048.1
1577; Citromicrobium sp. JLT1363 contig00009, whole genome shotgun
sequence; 341575924; NZ_AEUE01000009.1
1578; Acaryochloris sp. CCMEE 5410 contig00232, whole genome shotgun
sequence; 359367134; NZ_AFEJ01000154.1
1579; Stenotrophomonas maltophilia strain 419_SMAL
707_128228_1961615_4_{——}642_{——}523_, whole genome shotgun sequence;
896535166; NZ_JVHW01000017.1
1580; Streptomyces sp. S4, whole genome shotgun sequence; 358468601;
NZ_FR873700.1
1581; Pandoraea sp. SD6-2 scaffold29, whole genome shotgun sequence;
505733815; NZ_KB944444.1
1582; Mesorhizobium loti MAFF303099 DNA, complete genome; 57165207;
NC_002678.2
1583; Streptomyces avermitilis MA-4680 = NBRC 14893, complete genome;
162960844; NC_003155.4
1584; Thermobifida fusca TM51 contig028, whole genome shotgun sequence;
510814910; NZ_AOSG01000028.1
1585; Rhodobacter sphaeroides 2.4.1 chromosome 1, whole genome shotgun
sequence; 482849861; NZ_AKBU01000001.1
1586; Rhodospirillum rubrum F11, complete genome; 386348020; NC_017584.1
1587; Rhodospirillum rubrum F11, complete genome; 386348020; NC_017584.1
1588; Frankia sp. CcI6 CcI6DRAFT_scaffold_51.52, whole genome shotgun
sequence; 563312125; AYTZ01000052.1
1589; Roseobacter denitrificans OCh 114, complete genome; 110677421;
NC_008209.1
1590; Rhodobacter sphaeroides ATCC 17029 chromosome 1, complete
sequence; 126460778; NC_009049.1
1591; Rhodobacter sphaeroides ATCC 17025, complete genome; 146276058;
NC_009428.1
1592; Streptococcus suis SC84 complete genome, strain SC84; 253750923;
NC_012924.1
1593; Geobacter uraniireducens Rf4, complete genome; 148262085; NC_009483.1
1594; Sulfurovum sp. NBC37-1 genomic DNA, complete genome;
152991597; NC_009663.1
1595; Acaryochloris marina MBIC11017, complete genome; 158333233;
NC_009925.1
1596; Bacillus weihenstephanensis KBAB4, complete genome; 163938013;
NC_010184.1
1597; Caulobacter sp. K31 plasmid pCAUL01, complete sequence;
167621728; NC_010335.1
1598; Caulobacter sp. K31, complete genome; 167643973; NC_010338.1
1599; Candidatus Amoebophilus asiaticus 5a2, complete genome; 189501470;
NC_010830.1
1600; Stenotrophomonas maltophilia R551-3, complete genome; 194363778;
NC_011071.1
1601; Cyanothece sp. PCC 7425, complete genome; 220905643; NC_011884.1
1602; Chitinophaga pinensis DSM 2588, complete genome; 256419057;
NC_013132.1
1603; Haliangium ochraceum DSM 14365, complete genome; 262193326;
NC_013440.1
1604; Thermobaculum terrenum ATCC BAA-798 chromosome 2, complete
sequence; 269838913; NC_013526.1
1605; Xylanimonas cellulosilytica DSM 15894, complete genome;
269954810; NC_013530.1
1606; Stackebrandtia nassauensis DSM 44728, complete genome; 291297538;
NC_013947.1
1607; Sphingobium japonicum UT26S DNA, chromosome 1, complete
genome; 294009986; NC_014006.1
1608; Sphingobium japonicum UT26S plasmid pCHQ1 DNA, complete
genome; 294023656; NC_014007.1
1609; Butyrivibrio proteoclasticus B316 chromosome 1, complete sequence;
302669374; NC_014387.1
1610; Paenibacillus jamilae strain NS115 contig_27, whole genome shotgun
sequence; 970428876; NZ_LDRX01000027.1
1611; Frankia inefficax, complete genome; 312193897; NC_014666.1
1612; Asticcacaulis excentricus CB 48 chromosome 1, complete sequence;
315497051; NC_014816.1
1613; Terriglobus saanensis SP1PR4, complete genome; 320105246;
NC_014963.1
1614; Methanobacterium lacus strain AL-21, complete genome; 325957759;
NC_015216.1
1615; Marinomonas mediterranea MMB-1, complete genome; 326793322;
NC_015276.1
1616; Desulfobacca acetoxidans DSM 11109, complete genome; 328951746;
NC_015388.1
1617; Methanobacterium paludis strain SWAN1, complete genome;
333986242; NC_015574.1
1618; Frankia symbiont of Datisca glomerata, complete genome; 336176139;
NC_015656.1
1619; Halopiger xanaduensis SH-6 plasmid pHALXA01, complete genome;
336251750; NC_015658.1
1620; Mesorhizobium opportunistum WSM2075, complete genome;
337264537; NC_015675.1
1621; Runella slithyformis DSM 19594, complete genome; 338209545;
NC_015703.1
1622; Roseobacter litoralis Och 149, complete genome; 339501577;
NC_015730.1
1623; Streptomyces violaceusniger Tu 4113 plasmid pSTRVI01, complete
sequence; 345007457; NC_015951.1
1624; Streptomyces violaceusniger Tu 4113, complete genome; 345007964;
NC_015957.1
1625; Sphingobium sp. SYK-6 DNA, complete genome; 347526385;
NC_015976.1
1626; Chloracidobacterium thermophilum B chromosome 1, complete
sequence; 347753732; NC_016024.1
1627; Kitasatospora setae KM-6054 DNA, complete genome; 357386972;
NC_016109.1
1628; Streptomyces cattleya str. NRRL 8057 main chromosome, complete
genome; 357397620; NC_016111.1
1629; Legionella pneumophila subsp. pneumophila ATCC 43290, complete
genome; 378775961; NC_016811.1
1630; Rubrivivax gelatinosus IL144 DNA, complete genome; 383755859;
NC_017075.1
1631; Francisella cf. novicida 3523, complete genome; 387823583;
NC_017449.1
1632; Rhodospirillum rubrum F11, complete genome; 386348020;
NC_017584.1
1633; Actinoplanes sp. SE50/110, complete genome; 386845069;
NC_017803.1
1634; Legionella pneumophila subsp. pneumophila str. Lorraine chromosome,
complete genome; 397662556; NC_018139.1
1635; Emticicia oligotrophica DSM 17448, complete genome; 408671769;
NC_018748.1
1636; Streptomyces venezuelae ATCC 10712 complete genome; 408675720;
NC_018750.1
1637; Nostoc sp. PCC 7107, complete genome; 427705465; NC_019676.1
1638; Nostoc sp. PCC 7524, complete genome; 427727289; NC_019684.1
1639; Crinalium epipsammum PCC 9333, complete genome; 428303693;
NC_019753.1
1640; Thennobacillus composti KWC4, complete genome; 430748349;
NC_019897.1
1641; Mesorhizobium australicum WSM2073, complete genome; 433771415;
NC_019973.1
1642; Desulfocapsa sulfexigens DSM 10523, complete genome; 451945650;
NC_020304.1
1643; Rhodanobacter denitrificans strain 2APBS1, complete genome;
469816339; NC_020541.1
1644; Burkholderia thailandensis MSMB121 chromosome 1, complete
sequence; 488601775; NC_021173.1
1645; Streptomyces fulvissimus DSM 40593, complete genome; 488607535;
NC_021177.1
1646; Streptomyces davawensis strain JCM 4913 complete genome;
471319476; NC_020504.1
1647; Streptomyces davawensis strain JCM 4913 complete genome;
471319476; NC_020504.1
1648; Desulfotomaculum acetoxidans DSM 771, complete genome;
258513366; NC_013216.1
1649; Desulfotomaculum acetoxidans DSM 771, complete genome;
258513366; NC_013216.1
1650; Actinosynnema mirum DSM 43827, complete genome; 256374160;
NC_013093.1
1651; Bacillus cereus BAG2O-3 acfXF-supercont1.1, whole genome shotgun
sequence; 507017505; NZ_KB976530.1
1652; Bacillus cereus VD118 acrHo-supercont1.9, whole genome shotgun
sequence; 507035131; NZ_KB976800.1
1653; Bacillus cereus VDM053 acrGS-supcrcont1.7, whole genome shotgun
sequence; 507060152; NZ_KB976714.1
1654; Halomonas anticariensis FP35 = DSM 16096 strain FP35 Scaffold1,
whole genome shotgun sequence; 514429123; NZ_KE332377.1
1655; Halomonas anticariensis FP35 = DSM 16096 strain FP35 Scaffold1,
whole genome shotgun sequence; 514429123; NZ_KE332377.1
1656; Streptomyces sp. NRRL F-5639 contig75.1, whole genome shotgun
sequence; 664515060; NZ_JOGK01000075.1
1657; Acinetobacter gyllenbeigii MTCC 11365 contig1, whole genome
shotgun sequence; 514348304; NZ_ASQH01000001.1
1658; Streptomyces aurantiacus JA 4570 Seq17, whole genome shotgun
sequence; 514916021; NZ_AOPZ01000017.1
1659; Streptomyces aurantiacus JA 4570 Seq28, whole genome shotgun
sequence; 514916412; NZ_AOPZ01000028.1
1660; Streptomyces aurantiacus JA 4570 Seq63, whole genome shotgun
sequence; 514917321; NZ_AOPZ01000063.1
1661; Streptomyces aurantiacus JA 4570 Seq109, whole genome shotgun
sequence; 514918665; NZ_AOPZ01000109.1
1662; Paenibacillus polymyxa OSY-DF Contig136, whole genome shotgun
sequence; 484036841; NZ_AIPP01000136.1
1663; Fischerella muscicola SAG 1427-1 = PCC 73103 contig00215, whole
genome shotgun sequence; 484073367; NZ_AJLJ01000207.1
1664; Fischerella muscicola PCC 7414 contig00153, whole genome shotgun
sequence; 484075372; NZ_AJLK01000153.1
1665; Xanthomonas arboricola pv. corylina str. NCCB 100457 Contig50,
whole genome shotgun sequence; 507418017; NZ_APMC02000050.1
1666; Sphingobium xenophagum QYY contig015, whole genome shotgun
sequence; 484272664; NZ_AKIB01000015.1
1667; Pedobacter arcticus A12 Scaffold2, whole genome shotgun sequence;
484345004; NZ_JH947126.1
1668; Leptolyngbya boryana PCC 6306 LepboDRAFT_LPC.1, whole
genome shotgun sequence; 482909028; NZ_KB731324.1
1669; Fischerella sp. PCC 9339 PCC9339DRAFT_scaffold1.1, whole genome
shotgun sequence; 482909394; NZ_JH992898.1
1670; Mastigocladopsis repens PCC 10914 Mas10914DRAFT_scaffold1.1,
whole genome shotgun sequence; 482909462; NZ_JH992901.1
1671; Lactococcus garvieae Tac2 Tac2Contig_33, whole genome shotgun
sequence; 483258918; NZ_AMFE01000033.1
1672; Paenisporosarcina sp. TG-14 111.TG14.1_1, whole genome shotgun
sequence; 483299154; NZ_AMGD01000001.1
1673; Amphibacillus jilinensis Y1 Scaffold2, whole genome shotgun
sequence; 483992405; NZ_JH976435.1
1674; Alpha proteobacterium LLX12A LLX12A_contig00014, whole genome
shotgun sequence; 483996931; NZ_AMYX01000014.1
1675; Alpha proteobacterium LLX12A LLX12A_contig00026, whole genome
shotgun sequence; 483996974; NZ_AMYX01000026.1
1676; Alpha proteobacterium LLX12A LLX12A_contig00084, whole genome
shotgun sequence; 483997176; NZ_AMYX01000084.1
1677; Alpha proteobacterium L41A L41A_contig00002, whole genome
shotgun sequence; 483997957; NZ_AMYY01000002.1
1678; Nocardiopsis alba DSM 43377 contig_34, whole genome shotgun
sequence; 484007204; NZ_ANAC01000034.1
1679; Nocardiopsis halophila DSM 44494 contig_138, whole genome shotgun
sequence; 484007841; NZ_ANAD01000138.1
1680; Nocardiopsis halophila DSM 44494 contig_197, whole genome shotgun
sequence; 484008051; NZ_ANAD01000197.1
1681; Nocardiopsis halotolerans DSM 44410 contig_372, whole genome
shotgun sequence; 484016556; NZ_ANAX01000372.1
1682; Nocardiopsis lucentensis DSM 44048 contig_935, whole genome
shotgun sequence; 484021665; NZ_ANBC01000935.1
1683; Nocardiopsis alkaliphila YIM 80379 contig_111, whole genome
shotgun sequence; 484022237; NZ_ANBD01000111.1
1684; Nocardiopsis chromatogenes YIM 90109 contig_93, whole genome
shotgun sequence; 484026206; NZ_ANBH01000093.1
1685; Porphyrobacter sp. AAP82 Contig35, whole genome shotgun sequence;
484033307; NZ_ANFX01000035.1
1686; Blastomonas sp. AAP53 Contig8, whole genome shotgun sequence;
484033611; NZ_ANFZ01000008.1
1687; Blastomonas sp. AAP53 Contig14, whole genome shotgun sequence;
484033631; NZ_ANFZ01000014.1
1688; Paenibacillus sp. PAMC 26794 5104_29, whole genome shotgun
sequence; 484070054; NZ_ANHX01000029.1
1689; Oscillatoria sp. PCC 10802 Osc10802DRAFT_Contig7.7, whole
genome shotgun sequence; 484104632; NZ_KB235948.1
1690; Clostridium botulinum CB11/1-1 CB_contig00105, whole genome
shotgun sequence; 484141779; NZ_AORM01000006.1
1691; Actinopolyspora halophila DSM43834 ActhaDRAFT_contig1.1_C,
whole genome shotgun sequence; 484203522; NZ_AQUI01000002.1
1692; Asticcacaulis benevestitus DSM 16100 = ATCC BAA-896 strain DSM
16100 B060DRAFT_scaffold_12.13_C, whole genome shotgun sequence;
484226753; NZ_AQWM01000013.1
1693; Asticcacaulis benevestitus DSM 16100 = ATCC BAA-896 strain DSM
16100 B060DRAFT_scaffold_31.32_C, whole genome shotgun sequence;
484226810; NZ_AQWM01000032.1
1694; Streptomyces sp. FxanaC1 B074DRAFT_scaffold_1.2_C, whole
genome shotgun sequence; 484227180; NZ_AQWO01000002.1
1695; Streptomyces sp. FxanaC1 B074DRAFT_scaffold_7.8_C, whole
genome shotgun sequence; 484227195; NZ_AQWO01000008.1
1696; Smaragdicoccus niigatensis DSM 44881 = NBRC 103563 strain DSM
44881 F600DRAFT_scaffold00011.11_C, whole genome shotgun sequence;
484234624; NZ_AQXZ01000009.1
1697; Verrucomicrobium sp. 3C A37ADRAFT_scaffold1.1, whole genome
shotgun sequence; 483219562; NZ_KB901875.1
1698; Verrucomicrobium sp. 3C A37ADRAFT_scaffold1.1, whole genome
shotgun sequence; 483219562; NZ_KB901875.1
1699; Bradyrhizobium sp. WSM2793 A3ASDRAFT_scaffold_24.25, whole
genome shotgun sequence; 483314733; NZ_KB902785.1
1700; Streptomyces vitaminophilus DSM 41686 A3IGDRAFT_scaffold_10.11,
whole genome shotgun sequence; 483682977; NZ_KB904636.1
1701; Streptomyces sp. CcalMP-8W B053DRAFT_scaffold_17.18, whole
genome shotgun sequence; 483961830; NZ_KB890924.1
1702; Streptomyces sp. ScaeMP-e10 B061DRAFT_scaffold_0.1, whole
genome shotgun sequence; 483967534; NZ_KB891296.1
1703; Streptomyces sp. KhCrAH-244 B069DRAFT_scaffold_11.12, whole
genome shotgun sequence; 483969755; NZ_KB891596.1
1704; Streptomyces sp. HmicA12 B072DRAFT_scaffold_19.20, whole
genome shotgun sequence; 483972948; NZ_KB891808.1
1705; Streptomyces sp. MspMP-M5 B073DRAFT_scaffold_27.28, whole
genome shotgun sequence; 483974021; NZ_KB891893.1
1706; Bacillus mycoides strain Flugge 10206 DJ94.contig-100_16, whole
genome shotgun sequence; 727343482; NZ_JMQD01000030.1
1707; Streptomyces sp. CNY228 D330DRAFT_scaffold00011.11, whole
genome shotgun sequence; 484057944; NZ_KB898231.1
1708; Streptomyces sp. CNB091 D581DRAFT_scaffold00010.10, whole
genome shotgun sequence; 484070161; NZ_KB898999.1
1709; Sphingobium xenophagum NBRC 107872, whole genome shotgun
sequence; 483527356; NZ_BARE01000016.1
1710; Sphingobium xenophagum NBRC 107872, whole genome shotgun
sequence; 483532492; NZ_BARE01000100.1
1711; Bacillus oceanisediminis 2691 contig2644, whole genome shotgun
sequence; 485048843; NZ_ALEG01000067.1
1712; Bacillus sp. REN51N contig_2, whole genome shotgun sequence;
748816024; NZ_JXAB01000002.1
1713; Calothrix sp. PCC 7103 Cal7103DRAFT_CPM.6, whole genome
shotgun sequence; 485067373; NZ_KB217478.1
1714; Pseudanabaena sp. PCC 6802 Pse6802_scaffold_5, whole genome
shotgun sequence; 485067426; NZ_KB235914.1
1715; Actinopolyspora mortivallis DSM 44261 strain HS-1
ActmoDRAFT_scaffold1.1, whole genome shotgun sequence; 486324513;
NZ_KB913024.1
1716; Mesorhizobium huakuii 7653Rgenome; 657121522; CP006581.1
1717; Paenibacillus sp. HW567 B212DRAFT_scaffold1.1, whole genome
shotgun sequence; 486346141; NZ_KB910518.1
1718; Bacillus sp. 123MFChir2 H280DRAFT_scaffold00030.30, whole
genome shotgun sequence; 487368297; NZ_KB910953.1
1719; Streptomyces canus 299MFChir4.1 H293DRAFT_scaffold00032.32,
whole genome shotgun sequence; 487385965; NZ_KB911613.1
1720; Kribbella catacumbae DSM 19601 A3ESDRAFT_scaffold_7.8_C,
whole genome shotgun sequence; 484207511; NZ_AQUZ01000008.1
1721; Paenibacillus riograndensis SBR5 Contig78, whole genome shotgun
sequence; 485470216; NZ_A
1722; Nonomuraea coxensis DSM 45129 A3G7DRAFT_scaffold_4.5, whole
genome shotgun sequence; 483454700; NZ_KB903974.1
1723; Spirosoma spitsbergense DSM 19989 B157DRAFT_scaffold_76.77,
whole genome shotgun sequence; 483994857; NZ_KB893599.1
1724; Amycolatopsis alba DSM 44262 scaffold1, whole genome shotgun
sequence; 486330103; NZ_KB913032.1
1725; Amycolatopsis nigrescens CSC17Ta-90 AmyniDRAFT_Contig68.1_C,
whole genome shotgun sequence; 487404592; NZ_ARVW01000001.1
1726; Reyranella massiliensis 521, whole genome shotgun sequence;
484038067; NZ_HE997181.1
1727; Acidobacteriaceae bacterium KBS 83 G002DRAFT_scaffold00007.7,
whole genome shotgun sequence; 485076323; NZ_KB906739.1
1728; Novosphingobium lindaniclasticum LE124 contig147, whole genome
shotgun sequence; 544819688; NZ_ATHL01000147.1
1729; Actinobaculum sp. oral taxon 183 str. F0552 A_P1HMPREF0043-1.0_Cont1.1,
whole genome shotgun sequence; 541476958; AWSB01000006.1
1730; Sphingomonas-like bacterium B12, whole genome shotgun sequence;
484113405; NZ_BACX01000237.1
1731; Sphingomonas-like bacterium B12, whole genome shotgun sequence;
484113491; NZ_BACX01000258.1
1732; Thermoactinomyces vulgaris strain NRRL F-5595 F5595contig15.1,
whole genome shotgun sequence; 929862756; NZ_LGKI01000090.1
1733; Clostridium saccharobutylicum DSM 13864, complete genome;
550916528; NC_022571.1
1734; Butyrivibrio fibrisolvens AB2020 G616DRAFT_scaffold00015.15_C,
whole genome shotgun sequence; 551012921; NZ_ATVZ01000015.1
1735; Butyrivibrio sp. XPD2006 G590DRAFT_scaffold00008.8_C, whole
genome shotgun sequence; 551021553; NZ_ATVT01000008.1
1736; Butyrivibrio sp. AE3009 G588DRAFT_scaffold00030.30_C, whole
genome shotgun sequence; 551035505; NZ_ATVS01000030.1
1737; Acidobacteriaceae bacterium TAA166 strain TAA 166
H979DRAFT_scaffold_0.1_C, whole genome shotgun sequence; 551216990;
NZ_ATWD01000001.1
1738; Rothia aeria F0184 R_aeriaHMPREF0742-1.0_Cont136.4, whole
genome shotgun sequence; 551695014; AXZG01000035.1
1739; Klebsiella pneumoniae 4541-2 4541_2_67, whole genome shotgun
sequence; 657698352; NZ_JDWO01000067.1
1740; Klebsiella pneumoniae MGH 19 addTc-supercont1.2, whole genome
shotgun sequence; 556494858; NZ_KI535678.1
1741; Candidatus Halobonum tyrrellensis G22 contig00002, whole genome
shotgun sequence; 557371823; NZ_ASGZ01000002.1
1742; Asticcacaulis sp. AC466 contig00008, whole genome shotgun sequence;
557833377; NZ_AWGE01000008.1
1743; Asticcacaulis sp. AC466 contig00033, whole genome shotgun sequence;
557835508; NZ_AWGE01000033.1
1744; Asticcacaulis sp. YBE204 contig00005, whole genome shotgun
sequence; 557839256; NZ_AWGF01000005.1
1745; Asticcacaulis sp. YBE204 contig00010, whole genome shotgun
sequence; 557839714; NZ_AWGF01000010.1
1746; Streptomyces roseochromogenus subsp. oscitans DS 12.976 chromosome,
whole genome shotgun sequence; 566155502; NZ_CM002285.1
1747; Bacillus boroniphilus JCM 21738 DNA, contig: contig_6, whole
genome shotgun sequence; 571146044; BAUW01000006.1
1748; Mesorhizobium sp. LNHC232B00 scaffold0020, whole genome
shotgun sequence; 563561985; NZ_AYWP01000020.1
1749; Mesorhizobium sp. LNHC220B00 scaffold0002, whole genome
shotgun sequence; 563576979; NZ_AYWS01000002.1
1750; Mesorhizobium sp. LNHC221B00 scaffold0001, whole genome
shotgun sequence; 563570867; NZ_AYWR01000001.1
1751; Clostridium pasteurianum NRRL B-598, complete genome; 930593557;
NZ_CP011966.1
1752; Paenibacillus peoriae strain HS311, complete genome; 922052336;
NZ_CPO11512.1
1753; Magnetospirillum gryphiswaldense MSR-1 v2, complete genome;
568144401; NC_023065.1
1754; Streptococcus suis strain LS8F, whole genome shotgun sequence;
766589647; NZ_CEHJ01000007.1
1755; Bradyrhizobium sp. ARR65 BraARR65DRAFT_scaffold_9.10_C,
whole genome shotgun sequence; 639168743; NZ_AWZU01000010.1
1756; Paenibacillus sp. MAEPY2 contig7, whole genome shotgun sequence;
639451286; NZ_AWUK01000007.1
1757; Verrucomicrobia bacterium LP2A
G346DRAFT_scf7180000000012_quiver.2_C, whole genome shotgun
sequence; 640169055; NZ_JAFS01000002.1
1758; Verrucomicrobia bacterium LP2A
G346DRAFT_scf7180000000012_quiver.2_C, whole genome shotgun
sequence; 640169055; NZ_JAFS01000002.1
1759; Robbsia andropogonis Ba3549 160, whole genome shotgun sequence;
640451877; NZ_AYSW01000160.1
1760; Xanthomonas arboricola 3004 contig00003, whole genome shotgun
sequence; 640500871; NZ_AZQY01000003.1
1761; Bacillus mannanilyticus JCM 10596, whole genome shotgun sequence;
640600411; NZ_BAMO01000071.1
1762; Bacillus sp. H1a Contig1, whole genome shotgun sequence; 640724079;
NZ_AYMH01000001.1
1763; Enterococcus faecalis ATCC 4200 supercont1.2, whole genome shotgun
sequence; 239948580; NZ_GG670372.1
1764; Haloglycomyces albus DSM 45210 HalalDRAFT_chromosome1.1_C,
whole genome shotgun sequence; 644043488; NZ_AZUQ01000001.1
1765; Sphingomonas sanxanigenens NX02, complete genome; 749321911;
NZ_CP006644.1
1766; Kutzneria albida strain NRRL B-24060 contig305.1, whole genome
shotgun sequence; 662161093; NZ_JNYH01000515.1
1767; Kutzneria albida DSM 43870, complete genome; 754862786;
NZ_CP007155.1
1768; Paenibacillus sp. ICGEB2008 Contig_7, whole genome shotgun
sequence; 483624383; NZ_AMQU01000007.1
1769; Sphingobium barthaii strain KK22, whole genome shotgun sequence;
646529442; NZ_BATN01000092.1
1770; Paenibacillus polymyxa 1-43 S143_contig00221, whole genome
shotgun sequence; 647225094; NZ_ASRZ01000173.1
1771; Paenibacillus graminis RSA19 S2_contig00597, whole genome shotgun
sequence; 647256651; NZ_ASSG01000304.1
1772; Paenibacillus polymyxa TD94 STD94_contig00759, whole genome
shotgun sequence; 647274605; NZ_ASSA01000134.1
1773; Bacillus flexus T6186-2 contig_106, whole genome shotgun sequence;
647636934; NZ_JANV01000106.1
1774; Brevundimonas naejangsanensis strain B1 contig000018, whole genome
shotgun sequence; 647728918; NZ_JHOF01000018.1
1775; Sphingomonas-like bacterium B12, whole genome shotgun sequence;
484115568; NZ_BACX01000797.1
1776; Nocardiopsis potens DSM 45234 contig_25, whole genome shotgun
sequence; 484017897; NZ_ANBB01000025.1
1777; Nocardiopsis halotolerans DSM 44410 contig_26, whole genome
shotgun sequence; 484015294; NZ_ANAX01000026.1
1778; Nocardiopsis baichengensis YIM 90130 Scaffold15_1, whole genome
shotgun sequence; 484012558; NZ_ANAS01000033.1
1779; Nocardiopsis alba DSM 43377 contig_10, whole genome shotgun
sequence; 484007121; NZ_ANAC01000010.1
1780; Sphingomonas melonis DAPP-PG 224 Sphme3DRAFT_scaffold1.1,
whole genome shotgun sequence; 482984722; NZ_KB900605.1
1781; Acidobacteriaceae bacterium TAA166 strain TAA 166
H979DRAFT_scaffold_0.1_C, whole genome shotgun sequence; 551216990;
NZ_ATWD01000001.1
1782; Actinomadura oligospora ATCC 43269 P696DRAFT_scaffold00008.8_C,
whole genome shotgun sequence; 651281457; NZ_JADG01000010.1
1783; Butyrivibrio sp. XPD2002 G587DRAFT_scaffold00011.11, whole
genome shotgun sequence; 651381584; NZ_KE384117.1
1784; Bacillus sp. UNC437CL72CviS29 M014DRAFT_scaffold00009.9_C,
whole genome shotgun sequence; 651596980; NZ_AXVB01000011.1
1785; Butyrivibrio sp. FC2001 G601DRAFT_scaffold00001.1, whole genome
shotgun sequence; 651921804; NZ_KE384132.1
1786; Bacillus bogoriensis ATCC BAA-922 T323DRAFT_scaffold00008.8_C,
whole genome shotgun sequence; 651937013; NZ_JHYI01000013.1
1787; Fischerella sp. PCC 9431 Fis9431DRAFT_Scaffold1.2, whole genome
shotgun sequence; 652326780; NZ_KE650771.1
1788; Fischerella sp. PCC 9605 FIS9605DRAFT_scaffold2.2, whole genome
shotgun sequence; 652337551; NZ_KI912149.1
1789; Clostridium akagii DSM 12554 BR66DRAFT_scaffold00010.10_C,
whole genome shotgun sequence; 652488076; NZ_JMLK01000014.1
1790; Glomeribacter sp. 1016415 H174DRAFT_scaffold00001.1, whole
genome shotgun sequence; 652527059; NZ_KE384226.1
1791; Mesorhizobium sp. URHA0056 H959DRAFT_scaffold00004.4_C,
whole genome shotgun sequence; 652670206; NZ_AUEL01000005.1
1792; Mesorhizobium sp. URHA0056 H959DRAFT_scaffold00004.4_C,
whole genome shotgun sequence; 652670206; NZ_AUEL01000005.1
1793; Mesorhizobium loti R88b Meslo2DRAFT_Scaffold1.1, whole genome
shotgun sequence; 652688269; NZ_KI912159.1
1794; Mesorhizobium loti R88b Meslo2DRAFT_Scaffold1.1, whole genome
shotgun sequence; 652688269; NZ_KI912159.1
1795; Mesorhizobium ciceri WSM4083 MESCI2DRAFT_scaffold_0.1,
whole genome shotgun sequence; 652698054; NZ_KI912610.1
1796; Mesorhizobium sp. URHC0008 N549DRAFT_scaffold00001.1_C,
whole genome shotgun sequence; 652699616; NZ_JIAP01000001.1
1797; Mesorhizobium huakuii 7653R genome; 657121522; CP006581.1
1798; Mesorhizobium erdmanii USDA 3471 A3AUDRAFT_scaffold_7.8_C,
whole genome shotgun sequence; 652719874; NZ_AXAE01000013.1
1799; Mesorhizobium erdmanii USDA 3471 A3AUDRAFT_scaffold_7.8_C,
whole genome shotgun sequence; 652719874; NZ_AXAE01000013.1
1800; Mesorhizobium loti CJ3sym A3A9DRAFT_scaffold_25.26_C, whole
genome shotgun sequence; 652734503; NZ_AXAL01000027.1
1801; Cohnella thermotolerans DSM 17683 G485DRAFT_scaffold00003.3,
whole genome shotgun sequence; 652794305; NZ_KE386956.1
1802; Mesorhizobium sp. WSM3626 Mesw3626DRAFT_scaffold_6.7_C,
whole genome shotgun sequence; 652879634; NZ_AZUY01000007.1
1803; Mesorhizobium sp. WSM1293 MesloDRAFT_scaffold_4.5, whole
genome shotgun sequence; 652910347; NZ_KI911320.1
1804; Legionella pneumophila subsp. pneumophila strain ATCC 33155
contig032, whole genome shotgun sequence; 652971687; NZ_JFIN01000032.1
1805; Legionella pneumophila subsp. pneumophila strain ATCC 33154
Scaffold2, whole genome shotgun sequence; 653016013; NZ_KK074241.1
1806; Legionella pneumophila subsp. pneumophila strain ATCC 33823
Scaffold7, whole genome shotgun sequence; 653016661; NZ_KK074199.1
1807; Bacillus sp. URHB0009 H980DRAFT_scaffold00016.16_C, whole
genome shotgun sequence; 653070042; NZ_AUER01000022.1
1808; Lachnospira multipara MC2003 T520DRAFT_scaffold00007.7_C,
whole genome shotgun sequence; 653225243; NZ_JHWY01000011.1
1809; Rhodanobacter sp. OR87 RhoOR87DRAFT_scaffold_24.25_C, whole
genome shotgun sequence; 653308965; NZ_AXBJ01000026.1
1810; Rhodanobacter sp. OR92 RhoOR92DRAFT_scaffold_6.7_C, whole
genome shotgun sequence; 653321547; NZ_ATYF01000013.1
1811; Rhodanobacter sp. OR444
RHOOR444DRAFT_NODE_5_len_27336_cov_289_843719.5_C, whole
genome shotgun sequence; 653325317; NZ_ATYD01000005.1
1812; Rhodanobacter sp. OR444
RHOOR444DRAFT_NODE_39_len_52063_cov_320_872864.39, whole
genome shotgun sequence; 653330442; NZ_KE386531.1
1813; Bradyrhizobium sp. Aila-2 K288DRAFT_scaffold00086.86_C, whole
genome shotgun sequence; 653556699; NZ_AUEZ01000087.1
1814; Streptomyces sp. CNH099 B121DRAFT_scaffold_16.17_C, whole
genome shotgun sequence; 654239557; NZ_AZWL01000018.1
1815; Mastigocoleus testarum BC008 Contig-2, whole genome shotgun
sequence; 959926096; NZ_LMTZ01000085.1
1816; [Eubacterium] cellulosolvens LD2006 T358DRAFT_scaffold00002.2_C,
whole genome shotgun sequence; 654392970; NZ_JHXY01000005.1
1817; Caulobacter sp. URHA0033 H963DRAFT_scaffold00023.23_C, whole
genome shotgun sequence; 654573246; NZ_AUEO01000025.1
1818; Legionella pneumophila subsp. fraseri strain ATCC 35251 contig031,
whole genome shotgun sequence; 654928151; NZ_JFIG01000031.1
1819; Bacillus sp. FJAT-14578 Scaffold2, whole genome shotgun sequence;
654948246; NZ_KI632505.1
1820; Bacillus sp. 278922 107H622DRAFT_scaffold00001.1, whole genome
shotgun sequence; 654964612; NZ_KI911354.1
1821; Streptomyces sp. SolWspMP-sol2th B083DRAFT_scaffold_17.18_C,
whole genome shotgun sequence; 654969845; NZ_ARPF01000020.1
1822; Ruminococcus flavefaciens ATCC 19208 L870DRAFT_scaffold00001.1,
whole genome shotgun sequence; 655069822; NZ_KI912489.1
1823; Paenibacillus sp. UNCCL52 BR01DRAFT_scaffold00001.1, whole
genome shotgun sequence; 655095448; NZ_KK366023.1
1824; Paenibacillus taiwanensis DSM 18679 H509DRAFT_scaffold00010.10_C,
whole genome shotgun sequence; 655095554; NZ_AULE01000001.1
1825; Paenibacillus sp. UNC451MF BP97DRAFT_scaffold00018.18_C,
whole genome shotgun sequence; 655103160; NZ_JMLS01000021.1
1826; Desulfobulbus japonicus DSM 18378 G493DRAFT_scaffold00011.11_C,
whole genome shotgun sequence; 655133038; NZ_AUCV01000014.1
1827; Novosphingobium sp. B-7 scaffold147, whole genome shotgun
sequence; 514419386; NZ_KE148338.1
1828; Streptomyces flavidovirens DSM 40150 G412DRAFT_scaffold00009.9,
whole genome shotgun sequence; 655416831; NZ_KE386846.1
1829; Terasakiella pusilia DSM 6293 Q397DRAFT_scaffold00039.39_C,
whole genome shotgun sequence; 655499373; NZ_JHYO01000039.1
1830; Pseudoxanthomonas suwonensis J43 Psesu2DRAFT_scaffold_44.45_C,
whole genome shotgun sequence; 655566937; NZ_JAES01000046.1
1831; Salinarimonas rosea DSM 21201 G407DRAFT_scaffold00021.21_C,
whole genome shotgun sequence; 655990125; NZ_AUBC01000024.1
1832; Paenibacillus harenae DSM 16969 H581DRAFT_scaffold00004.4,
whole genome shotgun sequence; 656245934; NZ_KE383845.1
1833; Paenibacillus alginolyticus DSM 5050 = NBRC 15375 strain DSM 5050
G519DRAFT_scaffold00043.43_C, whole genome shotgun sequence;
656249802; NZ_AUGY01000047.1
1834; Bacillus sp. RP1137 contig_18, whole genome shotgun sequence;
657210762; NZ_AXZS01000018.1
1835; Streptomyces leeuwenhoekii strain C34(2013) c34_sequence_0501,
whole genome shotgun sequence; 657301257; NZ_AZSD01000480.1
1836; Brevundimonas bacteroides DSM 4726 Q333DRAFT_scaffold00004.4_C,
whole genome shotgun sequence; 657605746; NZ_JNIX01000010.1
1837; Bacillus thuringiensis LM1212 scaffold_08, whole genome shotgun
sequence; 657629081; NZ_AYPV01000024.1
1838; Lachnoclostridium phytofermentans KNHs212
B010DRAFT_scf7180000000004_quiver.1_C, whole genome shotgun
sequence; 657706549; NZ_JNLM01000001.1
1839; Paenibacillus polymyxa strain NRRL B-30509 contig00003, whole
genome shotgun sequence; 766607514; NZ_JTHO01000003.1
1840; Paenibacillus polymyxa strain WLY78 S6_contig00095, whole genome
shotgun sequence; 657719467; NZ_ALJV01000094.1
1841; Stenotrophomonas maltophilia RR-10 STMALcontig40, whole genome
shotgun sequence; 484978121; NZ_AGRB01000040.1
1842; [Scytonema hofmanni] UTEX 2349 Tol9009DRAFT_TPD.8, whole
genome shotgun sequence; 657935980; NZ_KK073768.1
1843; Caulobacter sp. UNC358MFTsu5.1 BR39DRAFT_scaffold00002.2_C,
whole genome shotgun sequence; 659864921; NZ_JONW01000006.1
1844; Sphingomonas sp. UNC305MFCol5.2 BR78DRAFT_scaffold00001.1_C,
whole genome shotgun sequence; 659889283; NZ_JOOE01000001.1
1845; Streptomyces monomycini strain NRRL B-24309 P063_Doro1_scaffold135,
whole genome shotgun sequence; 662059070; NZ_KL571162.1
1846; Streptomyces peruviensis strain NRRL ISP-5592 P181_Doro1_scaffold152,
whole genome shotgun sequence; 662097244; NZ_KL575165.1
1847; Streptomyces natalensis strain NRRL B-5314 P055_Doro1_scaffold13,
whole genome shotgun sequence; 662108422; NZ_KL570019.1
1848; Streptomyces natalensis ATCC 27448 Scaffold_33, whole genome
shotgun sequence; 764439507; NZ_JRKI01000027.1
1849; Streptomyces baarnensis strain NRRL B-2842 P144_Doro1_scaffold6,
whole genome shotgun sequence; 662129456; NZ_KL573544.1
1850; Streptomyces decoyicus strain NRRL ISP-5087 P056_Doro1_scaffold78,
whole genome shotgun sequence; 662133033; NZ_KL570321.1
1851; Streptomyces baarnensis strain NRRL B-2842 P144_Doro1_scaffold26,
whole genome shotgun sequence; 662135579; NZ_KL573564.1
1852; Streptomyces puniceus strain NRRL ISP-5083 contig3.1, whole genome
shotgun sequence; 663149970; NZ_JOBQ01000003.1
1853; Spirillospora albida strain NRRL B-3350 contig1.1, whole genome
shotgun sequence; 663122276; NZ_JOFJ01000001.1
1854; Streptomyces sp. NRRL S-481 P269_Doro1_scaffold20, whole genome
shotgun sequence; 664428976; NZ_KL585179.1
1855; Streptomyces sp. NRRL S-87 contig69.1, whole genome shotgun
sequence; 663169513; NZ_JO
1856; Streptomyces katrae strain NRRL B-16271 contig33.1, whole genome
shotgun sequence; 663300513; NZ_JNZY01000033.1
1857; Streptomyces katrae strain NRRL B-16271 contig37.1, whole genome
shotgun sequence; 663300941; NZ_JNZY01000037.1
1858; Streptomyces sp. NRRL B-3229 contig5.1, whole genome shotgun
sequence; 663316931; NZ_JOGP01000005.1
1859; Streptomyces griseus subsp. griseus strain NRRL F-2227 contig41.1,
whole genome shotgun sequence; 664325626; NZ_JOIT01000041.1
1860; Streptomyces roseoverticillatus strain NRRL B-3500 contig22.1, whole
genome shotgun sequence; 663372343; NZ_JOFL01000022.1
1861; Streptomyces roseoverticillatus strain NRRL B-3500 contig43.1, whole
genome shotgun sequence; 663373497; NZ_JOFL01000043.1
1862; Streptomyces rimosus subsp. rimosus strain NRRL WC-3924 contig19.1,
whole genome shotgun sequence; 663376433; NZ_JQBW01000019.1
1863; Streptomyces rimosus subsp. rimosus strain NRRL WC-3924 contig82.1,
whole genome shotgun sequence; 663379797; NZ_JOBW01000082.1
1864; Streptomyces sp. NRRL F-5917 contig68.1, whole genome shotgun
sequence; 663414324; NZ_JOHQ01000068.1
1865; Streptomyces sp. NRRL S-1448 contig134.1, whole genome shotgun
sequence; 663421576; NZ_JOGE01000134.1
1866; Allokutzneria albata strain NRRL B-24461 contig22.1, whole genome
shotgun sequence; 663596322; NZ_JOEF01000022.1
1867; Sphingobium sp. DC-2 ODE_45, whole genome shotgun sequence;
663818579; NZ_JNAC01000042.1
1868; Streptomyces aureocirculatus strain NRRL ISP-5386 contig11.1, whole
genome shotgun sequence; 664013282; NZ_JOAP01000011.1
1869; Streptomyces cyaneofuscatus strain NRRL B-2570 contig9.1, whole
genome shotgun sequence; 664021017; NZ_JOEM01000009.1
1870; Streptomyces aureocirculatus strain NRRL ISP-5386 contig49.1, whole
genome shotgun sequence; 664026629; NZ_JOAP01000049.1
1871; Streptomyces sclerotialus strain NRRL B-2317 contig7.1, whole genome
shotgun sequence; 664034500; NZ_JODX01000007.1
1872; Streptomyces anulatus strain NRRL B-2873 contig21.1, whole genome
shotgun sequence; 664049400; NZ_JOEZ01000021.1
1873; Streptomyces globisporus subsp. globisporus strain NRRL B-2709 contig24.1,
whole genome shotgun sequence; 664051798; NZ_JNZK01000024.1
1874; Streptomyces rimosus subsp. rimosus strain NRRL B-2660 contig14.1,
whole genome shotgun sequence; 664052786; NZ_JOES01000014.1
1875; Streptomyces rimosus subsp. rimosus strain NRRL B-2660 contig59.1,
whole genome shotgun sequence; 664061406; NZ_JOES01000059.1
1876; Streptomyces achromogenes subsp. achromogenes strain NRRL B-2120
contig2.1, whole genome shotgun sequence; 664063830; NZ_JODT01000002.1
1877; Streptomyces rimosus subsp. rimosus strain NRRL B-2660 contig124.1,
whole genome shotgun sequence; 664066234; NZ_JOES01000124.1
1878; Streptomyces albus subsp. albus strain NRRL B-2445 contig28.1, whole
genome shotgun sequence; 664095100; NZ_JOED01000028.1
1879; Streptomyces rimosus subsp. rimosus strain NRRL WC-3929 contig5.1,
whole genome shotgun sequence; 664104387; NZ_JOJJ01000005.1
1880; Streptomyces rimosus subsp. rimosus strain NRRL WC-3904 contig10.1,
whole genome shotgun sequence; 664126885; NZ_JOCQ01000010.1
1881; Streptomyces rimosus subsp. rimosus strain NRRL WC-3904
contig106.1, whole genome shotgun sequence; 664141810; NZ_JOCQ01000106.1
1882; Streptomyces griseus subsp. griseus strain NRRL F-5144 contig19.1,
whole genome shotgun sequence; 664184565; NZ_JOGA01000019.1
1883; Streptomyces sp. NRRL F-2295 P395contig79.1, whole genome
shotgun sequence; 926288193; NZ_LGCY01000146.1
1884; Streptomyces xiamenensis strain 318, complete genome; 921170702;
NZ_CP0099222
1885; Streptomyces griseus subsp. griseus strain NRRL F-5618 contig4.1,
whole genome shotgun sequence; 664233412; NZ_JOGN01000004.1
1886; Streptomyces lavenduligriseus strain NRRL ISP-5487 contig2.1, whole
genome shotgun sequence; 664244706; NZ_JOBD01000002.1
1887; Streptomyces lavenduligriseus strain NRRL ISP-5487 contig2.1, whole
genome shotgun sequence; 664244706; NZ_JOBD01000002.1
1888; Streptomyces sp. NRRL S-920 contig3.1, whole genome shotgun
sequence; 664245663; NZ_JODF01000003.1
1889; Streptomyces sp. NRRL S-337 contig41.1, whole genome shotgun
sequence; 664277815; NZ_JOIX01000041.1
1890; Streptomyces griseus strain S4-7 contig113, whole genome shotgun
sequence; 764464761; NZ_JYBE01000113.1
1891; Streptomyces sp. NRRL F-4474 contig32.1, whole genome shotgun
sequence; 664323078; NZ_JOIB01000032.1
1892; Streptomyces sp. NRRL S-475 contig32.1, whole genome shotgun
sequence; 664325162; NZ_JOJB01000032.1
1893; Streptomyces sp. NRRL S-646 contig23.1, whole genome shotgun
sequence; 664421883; NZ_JODC01000023.1
1894; Streptomyces sp. NRRL S-1813 contig13.1, whole genome shotgun
sequence; 664466568; NZ_JOHB01000013.1
1895; Streptomyces sp. NRRL WC-3773 contig2.1, whole genome shotgun
sequence; 664478668; NZ_JOJI01000002.1
1896; Streptomyces sp. NRRL WC-3773 contig36.1, whole genome shotgun
sequence; 664487325; NZ_JOJI01000036.1
1897; Streptomyces olivaceus strain NRRL B-3009 contig20.1, whole genome
shotgun sequence; 664523889; NZ_JOFH01000020.1
1898; Streptomyces ochraceiscleroticus strain NRRL ISP-5594 contig9.1,
whole genome shotgun sequence; 664540649; NZ_JOAX01000009.1
1899; Streptomyces sp. NRRL S-218 P205_Doro1_scaffold2, whole genome
shotgun sequence; 664556736; NZ_KL591003.1
1900; Streptomyces sp. NRRL S-218 P205_Doro1_scaffold34, whole genome
shotgun sequence; 664565137; NZ_KL591029.1
1901; Streptomyces olindensis strain DAUFPE 5622 103, whole genome
shotgun sequence; 739918964; NZ_JJOH01000097.1
1902; Streptomyces sp. NRRL S-623 contig14.1, whole genome shotgun
sequence; 665522165; NZ_JOJC01000016.1
1903; Streptomyces durhamensis strain NRRL B-3309 contig3.1, whole
genome shotgun sequence; 665586974; NZ_JNXR01000003.1
1904; Streptomyces durhamensis strain NRRL B-3309 contig23.1, whole
genome shotgun sequence; 665604093; NZ_JNXR01000023.1
1905; Streptomyces roseochromogenus subsp. oscitans DS 12.976 chromosome,
whole genome shotgun sequence; 566155502; NZ_CM002285.1
1906; Leptolyngbya sp. Heron Island J 50, whole genome shotgun sequence;
553739852; NZ_AWNH01000066.1
1907; Leptolyngbya sp. Heron Island J 50, whole genome shotgun sequence;
553739852; NZ_AWNH01000066.1
1908; Sphingobium lactosutens DS20 contig107, whole genome shotgun
sequence; 544811486; NZ_AIDP01000107.1
1909; Streptomyces sp. NRRL F-5123 contig24.1, whole genome shotgun
sequence; 671535174; NZ_JOHY01000024.1
1910; Bacillus sp. MB2021 T349DRAFT_scaffold00010.10_C, whole
genome shotgun sequence; 671553628; NZ_JNJJ01000011.1
1911; Lachnospira multipara LB2003 T537DRAFT_scaffold00010.10_C,
whole genome shotgun sequence; 671578517; NZ_JNKW01000011.1
1912; Clostridium drakei strain SL1 contig_20, whole genome shotgun
sequence; 692121046; NZ_JIBU02000020.1
1913; Candidatus Paracaedibacter symbiosus strain PRA9 Scaffold_1, whole
genome shotgun sequence; 692233141; NZ_JQAK01000001.1
1914; Stenotrophomonas maltophilia strain 53 contig_2, whole genome
shotgun sequence; 692316574; NZ_JRJA01000002.1
1915; Klebsiella variicola genome assembly Kv4880, contig BN1200_Contig_75,
whole genome shotgun sequence; 906292938; CXPB01000073.1
1916; Streptomyces alboviridis strain NRRL B-1579 contig_18.1, whole
genome shotgun sequence; 695845602; NZ_JNWU01000018.1
1917; Streptomyces sp. CNS654 CD02DRAFT_scaffold00023.23_C, whole
genome shotgun sequence; 695856316; NZ_JNLT01000024.1
1918; Streptomyces albus subsp. albus strain NRRL B-16041 contig26.1,
whole genome shotgun sequence; 695869320; NZ_JNWW01000026.1
1919; Streptomyces sp. JS01 contig2, whole genome shotgun sequence;
695871554; NZ_JPWW01000002.1
1920; Mesorhizobium ciceri CMG6 MescicDRAFT_scaffold_1.2_C, whole
genome shotgun sequence; 639162053; NZ_AWZS01000002.1
1921; Mesorhizobium japonicum R7A MesloDRAFT_Scaffold1.1, whole
genome shotgun sequence; 696358903; NZ_KI632510.1
1922; Stenotrophomonas maltophilia RA8, whole genome shotgun sequence;
493412056; NZ_CALM01000701.1
1923; Streptomyces griseus subsp. griseus strain NRRL B-2307 contig15.1,
whole genome shotgun sequence; 702684649; NZ_JNZI01000015.1
1924; Kitasatospora setae KM-6054 DNA, complete genome; 357386972;
NC_016109.1
1925; Streptomyces lydicus strain NRRL ISP-5461 contig41.1, whole genome
shotgun sequence; 702808005; NZ_JNZA01000041.1
1926; Streptomyces iakyrus strain NRRL ISP-5482 contig6.1, whole genome
shotgun sequence; 702914619; NZ_JNXI01000006.1
1927; Kibdelosporangium aridum subsp. largum strain NRRL B-24462 contig91.4,
whole genome shotgun sequence; 703243970; NZ_JNYM01001429.1
1928; Streptomyces galbus strain KCCM 41354 contig00021, whole genome
shotgun sequence; 716912366; NZ_JRHJ01000016.1
1929; Bacillus aryabhattai strain GZ03 contigl_scaffold1, whole genome
shotgun sequence; 723602665; NZ_JPIE01000001.1
1930; Bacillus mycoides FSL H7-687 Contig052, whole genome shotgun
sequence; 727271768; NZ_ASPY01000052.1
1931; Bacillus weihenstephanensis strain JAS 83/3 Bw_JAS-83/3_contig00005,
whole genome shotgun sequence; 910095435; NZ_JNLY01000005.1
1932; Sphingomonas sp. ERG5 Contig80, whole genome shotgun sequence;
734983422; NZ_JSXI01000079.1
1933; Lachnospira multipara ATCC 19207 G600DRAFT_scaffold00009.9_C,
whole genome shotgun sequence; 653218978; NZ_AUJG01000009.1
1934; Bacillus sp. 72 T409DRAFT_scf7180000000077_quiver.15_C, whole
genome shotgun sequence; 736160933; NZ_JQMI01000015.1
1935; Bacillus simplex BA2H3 scaffold2, whole genome shotgun sequence;
736214556; NZ_KN360955.1
1936; Dehalobacter sp. UNSWDHB Contig_139, whole genome shotgun
sequence; 544905305; NZ_AUUR01000139.1
1937; Actinomadura oligospora ATCC 43269 P696DRAFT_scaffold00008.8_C,
whole genome shotgun sequence; 651281457; NZ_JADG01000010.1
1938; Hyphomonas oceanitis SCH89 contig59, whole genome shotgun
sequence; 737569369; NZ_ARYL01000059.1
1939; Bacillus vietnamensis strain HD-02, whole genome shotgun sequence;
736762362; NZ_CCDN010000009.1
1940; Hyphomonas sp. CY54-11-8 contig4, whole genome shotgun sequence;
736764136; NZ_AWFD01000033.1
1941; Erythrobacter longus strain DSM 6997 contig9, whole genome shotgun
sequence; 736965849; NZ_JMIW01000009.1
1942; Caulobacter henricii strain CF287 EW90DRAFT_scaffold00023.23_C,
whole genome shotgun sequence; 737089868; NZ_JQJN01000025.1
1943; Caulobacter henricii strain YR570 EX13DRAFT_scaffold00022.22_C,
whole genome shotgun sequence; 737103862; NZ_JQJP01000023.1
1944; Calothrix sp. 336/3, complete genome; 821032128; NZ_CP011382.1
1945; Bacillus firmus DS1 scaffold33, whole genome shotgun sequence;
737350949; NZ_APVL01000034.1
1946; Bacillus hemicellulosilyticus JCM 9152, whole genome shotgun
sequence; 737360192; NZ_BAUU01000008.1
1947; Edaphobacter aggregans DSM 19364 Q363DRAFT_scaffold00032.32_C,
whole genome shotgun sequence; 737370143; NZ_JQKI01000040.1
1948; Bacillus sp. UNC322MFChir4.1 BR72DRAFT_scaffold00004.4, whole
genome shotgun sequence; 737456981; NZ_KN050811.1
1949; Hyphomonas oceanitis SCH89 contig20, whole genome shotgun
sequence; 737567115; NZ_ARYL01000020.1
1950; Hyphomonas oceanitis SCH89 contig59, whole genome shotgun
sequence; 737569369; NZ_ARYL01000059.1
1951; Halobacillus sp. BBL2006 cont444, whole genome shotgun sequence;
737576092; NZ_JRNX01000441.1
1952; Hyphomonas atlantica strain 22II1-22F38 contig10, whole genome
shotgun sequence; 737577234; NZ_AWFH01000002.1
1953; Hyphomonas atlantica strain 22II1-22F38 contig28, whole genome
shotgun sequence; 737580759; NZ_AWFH01000021.1
1954; Hyphomonas jannaschiana VP2 contig2, whole genome shotgun
sequence; 737608363; NZ_ARYJ01000002.1
1955; Bacillus akibai JCM 9157, whole genome shotgun sequence;
737696658; NZ_BAUV01000025.1
1956; Frankia sp. CeD CEDDRAFT_scaffold_22.23, whole genome shotgun
sequence; 737947180; NZ_JPGU01000023.1
1957; Clostridium butyricum strain NEC8, whole genome shotgun sequence;
960334134; NZ_CBYK010000003.1
1958; Clostridium butyricum AGR2140 G607DRAFT_scaffold00008.8_C,
whole genome shotgun sequence; 653632769; NZ_AUJN01000009.1
1959; Fusobacterium necrophorum BFTR-2 contig0075, whole genome
shotgun sequence; 737951550; NZ_JAAG01000075.1
1960; [Leptolyngbya] sp. JSC-1
Osccy1DRAFT_CYJSC1_DRAF_scaffold00069.1, whole genome shotgun
sequence; 738050739; NZ_KL662191.1
1961; Bradyrhizobium sp. WSM1743 YU9DRAFT_scaffold_1.2_C, whole
genome shotgun sequence; 653526890; NZ_AXAZ01000002.1
1962; Mesorhizobium sp. WSM3224 YU3DRAFT_scaffold_3.4_C, whole
genome shotgun sequence; 652912253; NZ_ATYO01000004.1
1963; Myxosarcina sp. GI1 contig_5, whole genome shotgun sequence;
738529722; NZ_JRFE01000006.1
1964; Novosphingobium resinovorum strain KF1 contig000002, whole
genome shotgun sequence; 738613868; NZ_JFYZ01000002.1
1965; Paenibacillus sp. FSL H7-689 Contig015, whole genome shotgun
sequence; 738716739; NZ_ASPU01000015.1
1966; Paenibacillus wynnii strain DSM 18334 unitig_2, whole genome
shotgun sequence; 738760618; NZ_JQCR01000002.1
1967; Paenibacillus sp. FSL R7-269 Contig022, whole genome shotgun
sequence; 738803633; NZ_ASPS01000022.1
1968; Paenibacillus pinihumi DSM 23905 = JCM 16419 strain DSM 23905
H583DRAFT_scaffold00005.5, whole genome shotgun sequence; 655115689;
NZ_KE383867.1
1969; Paenibacillus harenae DSM 16969 H581DRAFT_scaffold00002.2,
whole genome shotgun sequence; 655165706; NZ_KE383843.1
1970; Paenibacillus sp. FSL R7-277 Contig088, whole genome shotgun
sequence; 738841140; NZ_ASPX01000088.1
1971; Pseudonocardia acaciae DSM 45401 N912DRAFT_scaffold00002.2_C,
whole genome shotgun sequence; 655569633; NZ_JIAI01000002.1
1972; Amycolatopsis orientalis DSM 40040 = KCTC 9412 contig_32, whole
genome shotgun sequence; 499136900; NZ_ASJB01000015.1
1973; Sphingobium chlorophenolicum strain NBRC 16172 contig000025,
whole genome shotgun sequence; 739594477; NZ_JFHR01000025.1
1974; Sphingobium herbicidovorans NBRC 16415 contig000028, whole
genome shotgun sequence; 739610197; NZ_JFZA02000028.1
1975; Sphingobium sp. ba1 seq0028, whole genome shotgun sequence;
739622900; NZ_JPPQ01000069.1
1976; Sphingomonas paucimobilis strain EPA505 contig000016, whole
genome shotgun sequence; 739629085; NZ_JFYY01000016.1
1977; Sphingomonas paucimobilis strain EPA505 contig000027, whole
genome shotgun sequence; 739630357; NZ_JFYY01000027.1
1978; Sphingobium yanoikuyae ATCC 51230 supercont1.1, whole genome
shotgun sequence; 427407324; NZ_JH992904.1
1979; Sphingobium yanoikuyae strain B1 scaffold28, whole genome shotgun
sequence; 739656825; NZ_KL662220.1
1980; Sphingobium yanoikuyae strain B1 contig000002, whole genome
shotgun sequence; 739661773; NZ_JGVR01000002.1
1981; Sphingomonas wittichii strain YR128 EX04DRAFT_scaffold00050.50_C,
whole genome shotgun sequence; 739674258; NZ_JQMC01000050.1
1982; Sphingomonas sp. SKA58 scf_1100007010440, whole genome shotgun
sequence; 211594417; NZ_CH959308.1
1983; Sphingopyxis sp. LC363 contig1, whole genome shotgun sequence;
739699072; NZ_JNFC01000001.1
1984; Sphingopyxis sp. LC363 contig30, whole genome shotgun sequence;
739701660; NZ_JNFC01000024.1
1985; Sphingopyxis sp. LC363 contig5, whole genome shotgun sequence;
739702995; NZ_JNFC01000045.1
1986; Streptococcus salivarius strain NU10 contig_11, whole genome shotgun
sequence; 739748927; NZ_JJMT01000011.1
1987; Streptomyces griseoluteus strain NRRL ISP-5360 contig43.1, whole
genome shotgun sequence; 663180071; NZ_JOBE01000043.1
1988; Streptomyces griseorubens strain JSD-1 contig143, whole genome
shotgun sequence; 657284919; JJMG01000143.1
1989; Streptomyces avermitilis MA-4680 = NBRC 14893, complete genome;
162960844; NC_003155.4
1990; Streptomyces achromogenes subsp. achromogenes strain NRRL B-2120
contig2.1, whole genome shotgun sequence; 664063830; NZ_JODT01000002.1
1991; Streptomyces griseus subsp. griseus strain NRRL WC-3645 contig40.1,
whole genome shotgun sequence; 739830264; NZ_JOJE01000040.1
1992; Streptomyces scabiei strain NCPPB 4086 scf_65433_365.1, whole
genome shotgun sequence; 739854483; NZ_KL997447.1
1993; Streptomyces sp. FXJ7.023 Contig10, whole genome shotgun sequence;
510871397; NZ_APIV01000010.1
1994; Streptomyces sp. PRh5 contig001, whole genome shotgun sequence;
740097110; NZ_JABQ01000001.1
1995; Paenibacillus sp. FSL H7-0357, complete genome; 749299172;
NZ_CP009241.1
1996; Paenibacillus stellifer strain DSM 14472, complete genome; 753871514;
NZ_CP009286.1
1997; Burkholderia pseudomallei strain MSHR4018 scaffold2, whole genome
shotgun sequence; 740942724; NZ_KN323080.1
1998; Burkholderia sp. ABCPW 111 X946.contig-100_0, whole genome
shotgun sequence; 740958729; NZ_JPWT01000001.1
1999; Cupriavidus sp. IDO NODE_7, whole genome shotgun sequence;
742878908; NZ_JWMA01000006.1
2000; Paenibacillus polymyxa strain DSM 365 Contig001, whole genome
shotgun sequence; 746220937; NZ_JMIQ01000001.1
2001; Paenibacillus polymyxa strain CF05 genome; 746228615;
NZ_CP009909.1
2002; Novosphingobium malaysiense strain MUSC 273 Contig9, whole
genome shotgun sequence; 746241774; NZ_JTDI01000009.1
2003; Paenibacillus sp. IHB B 3415 contig_069, whole genome shotgun
sequence; 746258261; NZ_JUEI01000069.1
2004; Novosphingobium subterraneum strain DSM 12447 NJ75_contig000013,
whole genome shotgun sequence; 746288194; NZ_JRVC01000013.1
2005; Pandoraea sputorum strain DSM 21091, complete genome; 749204399;
NZ_CP010431.1
2006; Xanthomonas cannabis pv. cannabis strain NCPPB 3753 contig_67,
whole genome shotgun sequence; 746366822; NZ_JSZF01000067.1
2007; Xanthomonas arboricola pv. pruni MAFF 301420 strain MAFF301420,
whole genome shotgun sequence; 759376814; NZ_BAVC01000017.1
2008; Xanthomonas arboricola pv. celebensis strain NCPPB 1630 scf_49108_10.1,
whole genome shotgun sequence; 746486416; NZ_KL638873.1
2009; Xanthomonas arboricola pv. celebensis strain NCPPB 1832 scf_23466_141.1,
whole genome shotgun sequence; 746494072; NZ_KL638866.1
2010; Xanthomonas cannabis pv. cannabis strain NCPPB 2877 contig_94,
whole genome shotgun sequence; 746532813; NZ_JSZE01000094.1
2011; Sphingopyxis fribergensis strain Kp5.2, complete genome; 749188513;
NZ_CP009122.1
2012; Sphingopyxis fribergensis strain Kp5.2, complete genome; 749188513;
NZ_CP009122.1
2013; Streptomyces sp. 769, complete genome; 749181963; NZ_CP003987.1
2014; Hassallia byssoidea VB512170 scaffold_0, whole genome shotgun
sequence; 748181452; NZ_JTCM01000043.1
2015; Jeotgalibacillus malaysiensis strain D5 chromosome, complete genome;
749182744; NZ_CP009416.1
2016; Paenibacillus sp. FSL R7-0273, complete genome; 749302091;
NZ_CP009283.1
2017; Paenibacillus polymyxa strain Sb3-1, complete genome; 749204146;
NZ_CP010268.1
2018; Klebsiella pneumoniae CCHB01000016, whole genome shotgun
sequence; 749639368; NZ_CCHB01000016.1
2019; Streptomyces albus strain DSM 41398, complete genome; 749658562;
NZ_CP010519.1
2020; Streptomonospora alba strain YIM 90003 contig_9, whole genome
shotgun sequence; 749673329; NZ_JROO01000009.1
2021; Uncultured marine bacterium 463 clone EBAC080-L32B05 genomic
sequence; 41582259; AY458641.2
2022; Nocardiopsis chromatogenes YIM 90109 contig_59, whole genome
shotgun sequence; 484026076; NZ_ANBH01000059.1
2023; Paenibacillus dendritiformis C454 PDENDC1000064, whole genome
shotgun sequence; 374605177; NZ_AHKH01000064.1
2024; Streptomyces auratus AGR0001 Scaffold1_85, whole genome shotgun
sequence; 396995461; AJGV01000085.1
2025; Tolypothrix campylonemoides VB511288 scaffold_0, whole genome
shotgun sequence; 751565075; NZ_JXCB01000004.1
2026; Jeotgalibacillus soli strain P9 contig00009, whole genome shotgun
sequence; 751619763; NZ_JXRP01000009.1
2027; Cylindrospermum stagnate PCC 7417, complete genome; 434402184;
NC_019757.1
2028; Sphingopyxis alaskensis RB2256, complete genome; 103485498;
NC_008048.1
2029; Syntrophobotulus glycolicus DSM 8271, complete genome; 325288201;
NC_015172.1
2030; Novosphingobium aromaticivorans DSM 12444, complete genome;
87198026; NC_007794.1
2031; Novosphingobium sp. PP1Y Lpl large plasmid, complete replicon;
334133217; NC_015579.1
2032; Bacillus sp. 1NLA3E, complete genome; 488570484; NC_021171.1
2033; Burkholderia rhizoxinica HKI 454, complete genome; 312794749;
NC_014722.1
2034; Psychromonas ingrahamii 37, complete genome; 119943794;
NC_008709.1
2035; Streptococcus salivarius JIM8777 complete genome; 387783149;
NC_017595.1
2036; Actinosynnema mirum DSM 43827, complete genome; 256374160;
NC_013093.1
2037; Legionella pneumophila 2300/99 Alcoy, complete genome; 296105497;
NC_014125.1
2038; Paenibacillus sp. FSL R5-0912, complete genome; 754884871;
NZ_CP009282.1
2039; Streptomyces sp. NBRC 110027, whole genome shotgun sequence;
754788309; NZ_BBNO01000002.1
2040; Streptomyces sp. NBRC 110027, whole genome shotgun sequence;
754796661; NZ_BBNO01000008.1
2041; Paenibacillus sp. FSL R7-0331, complete genome; 754821094;
NZ_CP009284.1
2042; Kibdelosporangium sp. MJ126-NF4, whole genome shotgun sequence;
754819815; NZ_CDME01000002.1
2043; Paenibacillus camerounensis strain G4, whole genome shotgun
sequence; 754841195; NZ_CCDG010000069.1
2044; Paenibacillus borealis strain DSM 13188, complete genome;
754859657; NZ_CP009285.1
2045; Legionella pneumophila serogroup 1 strain TUM 13948, whole genome
shotgun sequence; 754875479; NZ_BAYQ01000013.1
2046; Streptacidiphilus neutrinimicus strain NBRC 100921, whole genome
shotgun sequence; 755016073; NZ_BBPO01000030.1
2047; Streptacidiphilus melanogenes strain NBRC 103184, whole genome
shotgun sequence; 755032408; NZ_BBPP01000024.1
2048; Streptacidiphilus anmyonensis strain NBRC 103185, whole genome
shotgun sequence; 755077919; NZ_BBPQ01000048.1
2049; Streptacidiphilus jiangxiensis strain NBRC 100920, whole genome
shotgun sequence; 755108320; NZ_BBPN01000056.1
2050; Mesorhizobium sp. ORS3359, whole genome shotgun sequence;
756828038; NZ_CCNC01000143.1
2051; Bacillus megaterium WSH-002, complete genome; 384044176;
NC_017138.1
2052; Aneurinibacillus migulanus strain Nagano E1 contig_36, whole genome
shotgun sequence; 928874573; NZ_LIXL01000208.1
2053; Sphingobium sp. Ant17 Contig_90, whole genome shotgun sequence;
759431957; NZ_JEMV01000094.1
2054; Pseudomonas sp. HMP271 Pseudomonas_HMP271_contig_7, whole
genome shotgun sequence; 759578528; NZ_JMFZ01000007.1
2055; Streptomyces luteus strain TRM 45540 Scaffold1, whole genome
shotgun sequence; 759659849; NZ_KN039946.1
2056; Streptomyces nodosus strain ATCC 14899 genome; 759739811;
NZ_CP009313.1
2057; Streptomyces fradiae strain ATCC 19609 contig0008, whole genome
shotgun sequence; 759752221; NZ_JNAD01000008.1
2058; Streptomyces bingchenggensis BCW-1, complete genome; 374982757;
NC_016582.1
2059; Streptomyces glaucescens strain GLA.O, complete genome; 759802587;
NZ_CP009438.1
2060; Novosphingobium sp. Rr 2-17 contig98, whole genome shotgun
sequence; 393773868; NZ_AKFJ01000097.1
2061; Nonomuraea Candida strain NRRL B-24552 contig27.1, whole genome
shotgun sequence; 759944049; NZ_JOAG01000029.1
2062; Nonomuraea Candida strain NRRL B-24552 contig28.1, whole genome
shotgun sequence; 759944490; NZ_JOAG01000030.1
2063; Nonomuraea Candida strain NRRL B-24552 contig42.1, whole genome
shotgun sequence; 759948103; NZ_JOAG01000045.1
2064; Paenibacillus polymyxa E681, complete genome; 864439741;
NC_014483.2
2065; Xanthomonas hortorum pv. carotae str. M081 chromosome, whole
genome shotgun sequence; 565808720; NZ_CM002307.1
2066; Novosphingobium sp. P6W scaffold3, whole genome shotgun sequence;
763092879; NZ_JXZE01000003.1
2067; Novosphingobium sp. P6W scaffold9, whole genome shotgun sequence;
763095630; NZ_JXZE01000009.1
2068; Sphingomonas hengshuiensis strain WHSC-8, complete genome;
764364074; NZ_CP010836.1
2069; Streptomyces ahygroscopicus subsp. wuyiensis strain CK-15 contig3,
whole genome shotgun sequence; 921220646; NZ_JXYI02000059.1
2070; Streptomyces cyaneogriseus subsp. noncyanogenus strain NMWT 1,
complete genome; 764487836; NZ_CP010849.1
2071; Bacillus subtilis subsp. spizizenii RFWG1A4 contig00010, whole
genome shotgun sequence; 764657375; NZ_AJHM01000010.1
2072; Mastigocladus laminosus UU774 scaffold_22, whole genome shotgun
sequence; 764671177; NZ_JXUO1000139.1
2073; Moorea producens 3L scf52052, whole genome shotgun sequence;
332710285; NZ_GL890953.1
2074; Streptomyces iranensis genome assembly Siranensis, scaffold
SCAF00002; 765016627; NZ_LK022849.1
2075; Risungbinella massiliensis strain GD1, whole genome shotgun sequence;
765315585; NZ_LN812103.1
2076; Sphingobium sp. YBL2, complete genome; 765344939; NZ_CP010954.1
2077; Streptococcus suis strain LS5J, whole genome shotgun sequence;
765394696; NZ_CEEZ01000028.1
2078; Streptococcus suis strain LS8I, whole genome shotgun sequence;
766595491; NZ_CEHM01000004.1
2079; Thalassospira sp. HJ NODE_2, whole genome shotgun sequence;
766668420; NZ_JYII01000010.1
2080; Frankia sp. CpI1-P FF86_1013, whole genome shotgun sequence;
946950294; NZ_LJJX01000013.1
2081; Streptococcus suis strain B28P, whole genome shotgun sequence;
769231516; NZ_CDTB01000010.1
2082; Streptomyces sp. NRRL F-4428 contig40.2, whole genome shotgun
sequence; 772774737; NZ_JYJI01000131.1
2083; Bacterium endosymbiont of Mortierella elongata FMR23-6, whole
genome shotgun sequence; 779889750; NZ_DF850521.1
2084; Streptomyces sp. FxanaA7 F611DRAFT_scaffold00041.41_C, whole
genome shotgun sequence; 780340655; NZ_LACL01000054.1
2085; Streptomyces rubellomurinus strain ATCC 31215 contig-63, whole
genome shotgun sequence; 783211546; NZ_JZKH01000064.1
2086; Streptomyces rubellomurinus subsp. indigofems strain ATCC 31304
contig-55, whole genome shotgun sequence; 783374270; NZ_JZKG01000056.1
2087; Bacillus sp. UMTAT18 contig000011, whole genome shotgun
sequence; 806951735; NZ_JSFD01000011.1
2088; Paenibacillus wulumuqiensis strain Y24 Scaffold4, whole genome
shotgun sequence; 808051893; NZ_KQ040793.1
2089; Paenibacillus dauci strain H9 Scaffold3, whole genome shotgun
sequence; 808064534; NZ_KQ040798.1
2090; Paenibacillus algorifonticola strain XJ259 Scaffold20_1, whole genome
shotgun sequence; 808072221; NZ_LAQO01000025.1
2091; Xanthomonas campestris strain 17, complete genome; 810489403;
NZ_CP011256.1
2092; Bacillus sp. SA1-12 scf7180000003378, whole genome shotgun
sequence; 817541164; NZ_LATZ01000026.1
2093; Spirosoma radiotolerans strain DG5A, complete genome; 817524426;
NZ_CP010429.1
2094; Streptomyces lydicus A02, complete genome; 822214995; NZ_CP007699.1
2095; Streptomyces lydicus A02, complete genome; 822214995; NZ_CP007699.1
2096; Bacillus cereus strain B4147 NODE_5, whole genome shotgun
sequence; 822530609; NZ_LCYN01000004.1
2097; Xanthomonas pisi DSM 18956 Contig_28, whole genome shotgun
sequence; 822535978; NZ_JPLE01000028.1
2098; Erythrobacter luteus strain KA37 contig1, whole genome shotgun
sequence; 822631216; NZ_LBHB01000001.1
2099; Xanthomonas arboricola strain CFBP 7634 Xarjug-CFBP7634-G11,
whole genome shotgun sequence; 825139250; NZ_JZEH01000001.1
2100; Xanthomonas arboricola strain CFBP 7651 Xarjug-CFBP7651-G11,
whole genome shotgun sequence; 825156557; NZ_JZEI01000001.1
2101; Luteimonas sp. FCS-9 scf7180000000225, whole genome shotgun
sequence; 825314716; NZ_LASZ01000002.1
2102; Streptomyces sp. KE1 Contig11, whole genome shotgun sequence;
82535362ENZ_LAYX01000011.1
2103; Streptomyces sp. M10 Scaffold2, whole genome shotgun sequence;
835355240; NZ_KN549147.1
2104; Xanthomonas cannabis pv. phaseoli strain Nyagatare scf_52938_7,
whole genome shotgun sequence; 835885587; NZ_KN265462.1
2105; Bacillus aryabhattai strain T61 Scaffold1, whole genome shotgun
sequence; 836596561; NZ_KQ087173.1
2106; Paenibacillus sp. TCA20, whole genome shotgun sequence; 843088522;
NZ_BBIW01000001.1
2107; Bacillus circulans strain RIT379 contig11, whole genome shotgun
sequence; 844809159; NZ_LDPH01000011.1
2108; Ornithinibacillus californiensis strain DSM 16628 contig_22, whole
genome shotgun sequence; 849059098; NZ_LDUE01000022.1
2109; Bacillus pseudalcaliphilus strain DSM 8725 super11, whole genome
shotgun sequence; 849078078; NZ_LFJO01000006.1
2110; Bacillus aryabhattai strain LK25 16, whole genome shotgun sequence;
850356871; NZ_LDWN01000016.1
2111; Methanobacterium arcticum strain M2 EI99DRAFT_scaffold00005.5_C,
whole genome shotgun sequence; 851140085; NZ_JQKN01000008.1
2112; Methanobacterium sp. SMA-27 DL91DRAFT_unitig_0_quiver.1_C,
whole genome shotgun sequence; 851351157; NZ_JQLY01000001.1
2113; Cellulomonas sp. A375-1 contig_129, whole genome shotgun sequence;
856992287; NZ_LFKW01000127.1
2114; Streptomyces sp. HNS054 contig28, whole genome shotgun sequence;
860547590; NZ_LDZX01000028.1
2115; Bacillus cereus strain RIMV BC 126 212, whole genome shotgun
sequence; 872696015; NZ_LABO01000035.1
2116; Sphingomonas sp. MEA3-1 contig00021, whole genome shotgun
sequence; 873296042; NZ_LECE01000021.1
2117; Sphingomonas sp. MEA3-1 contig00040, whole genome shotgun
sequence; 873296160; NZ_LECE01000040.1
2118; Bacillus sp. 220_BSPC 1447_75439_1072255, whole genome shotgun
sequence; 880954155; NZ_JVPL01000109.1
2119; Bacillus sp. 522_BSPC 2470_72498_1083579_594_{——}. . ._522_, whole
genome shotgun sequence; 880997761; NZ_JVDT01000118.1
2120; Streptomyces ipomoeae 91-03 gcontig_1108499710267, whole genome
shotgun sequence; 429195484; NZ_AEJC01000118.1
2121; Scytonema tolypothrichoides VB-61278 scaffold_6, whole genome
shotgun sequence; 890002594; NZ_JXCA01000005.1
2122; Erythrobacter atlanticus strain s21-N3, complete genome; 890444402;
NZ_CP011310.1
2123; Sphingobium yanoikuyae strain SHJ scaffold12, whole genome shotgun
sequence; 893711343; NZ_KQ235994.1
2124; Sphingobium yanoikuyae strain SHJ scaffold33, whole genome shotgun
sequence; 893711364; NZ_KQ236015.1
2125; Sphingobium yanoikuyae strain SHJ scaffold47, whole genome shotgun
sequence; 893711378; NZ_KQ236029.1
2126; Stenotrophomonas maltophilia strain 544_SMAL
1161_223966_2976806_599_{——}. . ._882_, whole genome shotgun sequence;
896492362; NZ_JVCU01000107.1
2127; Stenotrophomonas maltophilia strain 131_SMAL
1126_236170_8501292_717_{——}. . ._1018_, whole genome shotgun sequence;
896520167; NZ_JVUI01000038.1
2128; Stenotrophomonas maltophilia stain 951_SMAL 71_125859_2268311,
whole genome shotgun sequence; 896567682; NZ_JUMH01000022.1
2129; Stenotrophomonas maltophilia strain OC194 contig_98, whole genome
shotgun sequence; 930169273; NZ_LJJH01000098.1
2130; Streptococcus pseudopneumoniae stain 445_SPSE
347_91401_2272315_318_{——}. . ._319_, whole genome shotgun sequence;
896667361; NZ_JVGV01000030.1
2131; Streptomyces caatingaensis strain CMAA 1322 contig02, whole genome
shotgun sequence; 906344334; NZ_LFXA01000002.1
2132; Streptomyces caatingaensis strain CMAA 1322 contig02, whole genome
shotgun sequence; 906344334; NZ_LFXA01000002.1
2133; Streptomyces caatingaensis strain CMAA 1322 contig07, whole genome
shotgun sequence; 906344339; NZ_LFXA01000007.1
2134; Sphingopyxis alaskensis RB2256, complete genome; 103485498; NC_008048.1
2135; Sphingomonas wittichii RW1, complete genome; 148552929; NC_009511.1
2136; Caulobacter sp. K31, complete genome; 167643973; NC_010338.1
2137; Asticcacaulis excentricus CB 48 chromosome 2, complete sequence;
315499382; NC_014817.1
2138; Nocardiopsis dassonvillei subsp. dassonvillei DSM 43111 chromosome
1, complete sequence; 297558985; NC_014210.1
2139; Streptomyces wadayamensis strain A23 LGO_A23_AS7_CO0257,
whole genome shotgun sequence; 910050821; NZ_JHDU01000034.1
2140; Tolypothrix bouteillei VB521301 scaffold_1, whole genome shotgun
sequence; 910242069; NZ_JHEG02000048.1
2141; Silvibacterium bohemicum strain S15 contig_3, whole genome shotgun
sequence; 910257956; NZ_LBHJ01000003.1
2142; Silvibacterium bohemicum strain S15 contig_3, whole genome shotgun
sequence; 910257956; NZ_LBHJ01000003.1
2143; Silvibacterium bohemicum strain S15 contig_30, whole genome shotgun
sequence; 910257973; NZ_LBHJ01000020.1
2144; Streptomyces sp. NRRL WC-3773 contig11.1, whole genome shotgun
sequence; 664481891; NZ_JOJI01000011.1
2145; Streptomyces peucetius strain NRRL WC-3868 contig49.1, whole
genome shotgun sequence; 665671804; NZ_JOCK01000052.1
2146; Xanthomonas citri pv. mangiferaeindicae LMG 941, whole genome
shotgun sequence; 381171950; NZ_CAHO01000029.1
2147; Mesorhizobium sp. L2C084A000 scaffold0007, whole genome shotgun
sequence; 563938926; NZ_AYWX01000007.1
2148; Erythrobacter citreus LAMA 915 Contig13, whole genome shotgun
sequence; 914607448; NZ_JYNE01000028.1
2149; Bacillus flexus strain Riq5 contig_32, whole genome shotgun sequence;
914730676; NZ_LFQJ01000032.1
2150; Rhodanobacter thiooxydans LCS2 contig057, whole genome shotgun
sequence; 389809081; NZ_AJXW01000057.1
2151; Frankia alni str. ACN14A chromosome, complete sequence;
111219505; NC_008278.1
2152; Novosphingobium sp. PP1Y main chromosome, complete replicon;
334139601; NC_015580.1
2153; Salinibacter ruber M8 chromosome, complete genome; 294505815;
NC_014032.1
2154; Nocardiopsis salina YIM 90010 contig_87, whole genome shotgun
sequence; 484023389; NZ_ANBF01000087.1
2155; Kitasatospora setae KM-6054 DNA, complete genome; 357386972;
NC_016109.1
2156; Arthrobacter sp. 161MFSha2.1 C567DRAFT_scaffold00006.6, whole
genome shotgun sequence; 484021228; NZ_KB895788.1
2157; Lamprocystis purpurea DSM 4197 A39ODRAFT_scaffold_0.1, whole
genome shotgun sequence; 483254584; NZ_KB902362.1
2158; Streptomyces sp. ATexAB-D23 B082DRAFT_scaffold_0.1, whole
genome shotgun sequence; 483975550; NZ_KB892001.1
2159; Lunatimonas lonarensis strain AK24 S14_contig_18, whole genome
shotgun sequence; 499123840; NZ_AQHR01000021.1
2160; Amycolatopsis benzoatilytica AK 16/65 AmybeDRAFT_scaffold1.1,
whole genome shotgun sequence; 486399859; NZ_KB912942.1
2161; Nocardia transvalensis NBRC 15921, whole genome shotgun sequence;
485125031; NZ_BAGL01000055.1
2162; Sphingomonas sp. YL-JM2C contig056, whole genome shotgun
sequence; 661300723; NZ_ASTM01000056.1
2163; Butyrivibrio sp. XBB1001 G631DRAFT_scaffold00005.5_C, whole
genome shotgun sequence; 651376721; NZ_AUKA01000006.1
2164; Butyrivibrio fibrisolvens MD2001 G635DRAFT_scaffold00033.33_C,
whole genome shotgun sequence; 652963937; NZ_AUKD01000034.1
2165; Butyrivibrio sp.NC3005 G634DRAFT_scaffold00001.1, whole
genome shotgun sequence; 651394394; NZ_KE384206.1
2166; Shimazuella kribbensis DSM 45090 A3GQDRAFT_scaffold_0.1_C,
whole genome shotgun sequence; 655370026; NZ_ATZF01000001.1
2167; Shimazuella kribbensis DSM 45090 A3GQDRAFT_scaffold_5.6_C,
whole genome shotgun sequence; 655371438; NZ_ATZF01000006.1
2168; Desulfobulbus mediterraneus DSM 13871 G494DRAFT_scaffold00028.28_C,
whole genome shotgun sequence; 655138083; NZ_AUCW01000035.1
2169; Cohnella thermotolerans DSM 17683 G485DRAFT_scaffold00041.41_C,
whole genome shotgun sequence; 652787974; NZ_AUCP01000055.1
2170; Azospirillum halopraeferens DSM 3675 G472DRAFT_scaffold00039.39_C,
whole genome shotgun sequence; 655967838; NZ_AUCF01000044.1
2171; Bacillus kribbensis DSM 17871 H539DRAFT_scaffold00003.3, whole
genome shotgun sequence; 651983111; NZ_KE387239.1
2172; Leptolyngbya sp. Heron Island J 67, whole genome shotgun sequence;
553740975; NZ_AWNH01000084.1
2173; Streptomyces sp. GXT6 genomic scaffold Scaffold4, whole genome
shotgun sequence; 654975403; NZ_KI601366.1
2174; Promicromonospora kroppenstedtii DSM 19349 ProkrDRAFT_PKA.71,
whole genome shotgun sequence; 739097522; NZ_KI911740.1
2175; Bacillus sp. J37 BacJ37DRAFT_scaffold_0.1_C, whole genome
shotgun sequence; 651516582; NZ_JAEK01000001.1
2176; Prevotella oryzae DSM 17970 XylorDRAFT_XOA.1, whole genome
shotgun sequence; 738999090; NZ_KK073873.1
2177; Sphingobium sp. Antl7 Contig 45, whole genome shotgun sequence;
759429528; NZ_JEMV01000036.1
2178; Rubellimicrobium mesophilum DSM 19309 scaffold23, whole genome
shotgun sequence; 739419616; NZ_KK088564.1
2179; Butyrivibrio sp. MC2021 T359DRAFT_scaffold00010.10_C, whole
genome shotgun sequence; 651407979; NZ_JHXX01000011.1
2180; Clostridium beijerinckii HUN142 T483DRAFT_scaffold00004.4, whole
genome shotgun sequence; 652494892; NZ_KK211337.1
2181; Streptomyces sp. Tu 6176 scaffold00003, whole genome shotgun
sequence; 740044478; NZ_KK106990.1
2182; Novosphingobium resinovorum strain KF1 contig000008, whole
genome shotgun sequence; 738615271; NZ_JFYZ01000008.1
2183; Novosphingobium resinovorum strain KF1 contig000015, whole
genome shotgun sequence; 738617000; NZ_JFYZ01000015.1
2184; Hyphomonas chukchiensis strain BH-BN04-4 contig29, whole genome
shotgun sequence; 736736050; NZ_AWFG01000029.1
2185; Thioclava dalianensis strain DLFJ1-1 contig2, whole genome shotgun
sequence; 740220529; NZ_JHEH01000002.1
2186; Thioclava indica strain DT23-4 contig29, whole genome shotgun
sequence; 740292158; NZ_AUNB01000028.1
2187; Streptomyces albus subsp. albus strain NRRL B-1811 contig32.1, whole
genome shotgun sequence; 665618015; NZ_JODR01000032.1
2188; Kitasatospora sp. MBT66 scaffold3, whole genome shotgun sequence;
759755931; NZ_JAIY01000003.1
2189; Sphingomonas sp. DC-6 scaffold87, whole genome shotgun sequence;
662140302; NZ_JMUB01000087.1
2190; Sphingobium chlorophenolicum strain NBRC 16172 contig000062,
whole genome shotgun sequence; 739598481; NZ_JFHR01000062.1
2191; Nocardia sp. NRRL WC-3656 contig2.1, whole genome shotgun
sequence; 663737675; NZ_JOJF01000002.1
2192; Streptomyces flavochromogenes strain NRRL B-2684 contig8.1, whole
genome shotgun sequence; 663317502; NZ_JNZO01000008.1
2193; Bacillus indicus strain DSM 16189 Contig01, whole genome shotgun
sequence; 737222016; NZ_JNVC02000001.1
2194; Streptomyces bicolor strain NRRL B-3897 contig42.1, whole genome
shotgun sequence; 671498318; NZ_JOFR01000042.1
2195; Streptomyces sp. NRRL WC-3719 contig152.1, whole genome shotgun
sequence; 665536304; NZ_JOCD01000152.1
2196; Streptomyces sp. NRRL F-5053 contig1.1, whole genome shotgun
sequence; 664356765; NZ_JOHT01000001.1
2197; Streptomyces sp. NRRL S-1868 contig54.1, whole genome shotgun
sequence; 664360925; NZ_JQGD01000054.1
2198; Streptomyces hygroscopicus subsp. hygroscopicus strain NRRL B-1477
contig8.1, whole genome shotgun sequence; 664299296; NZ_JOIK01000008.1
2199; Desulfobacter vibriofonnis DSM 8776 Q366DRAFT_scaffold00036.35_C,
whole genome shotgun sequence; 737257311; NZ_JQKJ01000036.1
2200; Brevundimonas sp. EAKA contig5, whole genome shotgun sequence;
737322991; NZ_JMQR01000005.1
2201; Brevundimonas sp. EAKA contig5, whole genome shotgun sequence;
73732299ENZ_JMQR01000005.1
2202; Actinokineospora spheciospongiae strain EG49 contig1268_1, whole
genome shotgun sequence; 737301464; NZ_AYXG01000139.1
2203; Sphingobium sp. ba1 seq0028, whole genome shotgun sequence;
739622900; NZ_JPPQ01000069.1
2204; Rotlria dentocariosa strain C6B contig_5, whole genome shotgun
sequence; 739372122; NZ_JQHE01000003.1
2205; Rhodococcus fascians A21d2 contig10, whole genome shotgun
sequence; 739287390; NZ_JMFA01000010.1
2206; Rhodococcus fascians LMG 3625 contig38, whole genome shotgun
sequence; 694033726; NZ_JMEM01000016.1
2207; Sphingopyxis sp. MWB1 contig00002, whole genome shotgun
sequence; 696542396; NZ_JQFJ01000002.1
2208; Sphingobium yanoikuyae strain B1 scaffold1, whole genome shotgun
sequence; 739650776; NZ_KL662193.1
2209; Lysobacter daejeonensis GH1-9 contig23, whole genome shotgun
sequence; 738180952; NZ_AVPU01000014.1
2210; Sphingomonas sp. 35-24ZXX contig11_scaffold4, whole genome
shotgun sequence; 728827031; NZ_JROG01000008.1
2211; Sphingomonas sp. 37zxx contig3_scaffold2, whole genome shotgun
sequence; 728813405; NZ_JROH01000003.1
2212; Actinoalloteichus spitiensis RMV-1378 Contig406, whole genome
shotgun sequence; 483112234; NZ_AGVX02000406.1
2213; Alistipes sp. ZOR0009 L990_140, whole genome shotgun sequence;
835319962; NZ_JTLD01000119.1
2214; Sphingopyxis sp. LC363 contig36, whole genome shotgun sequence;
739702045; NZ_JNFC01000030.1
2215; Sphingopyxis sp. LC81 contig24, whole genome shotgun sequence;
739659070; NZ_JNFD01000017.1
2216; Sphingomonas sp. Ant H11 contig_149, whole genome shotgun
sequence; 730274767; NZ_JSBN01000149.1
2217; Novosphingobium malaysiense strain MUSC 273 Contig11, whole
genome shotgun sequence; 746242072; NZ_JTDI01000011.1
2218; Novosphingobium subterraneum strain DSM 12447 NJ75 contig000028,
whole genome shotgun sequence; 746290581; NZ_JRVC01000028.1
2219; Brevundimonas nasdae strain TPW30 Contig_13, whole genome
shotgun sequence; 746187665; NZ_JWSY01000013.1
2220; Desulfosporosinus youngiae DSM 17734 chromosome, whole genome
shotgun sequence; 374578721; NZ_CM001441.1
2221; Rivularia sp. PCC 7116, complete genome; 427733619; NC_019678.1
2222; Gorillibacterium massiliense strain G5, whole genome shotgun
sequence; 750677319; NZ_CBQR020000171.1
2223; Nonomuraea Candida strain NRRL B-24552 contig8.1, whole genome
shotgun sequence; 759934284; NZ_JOAG01000009.1
2224; Mesorhizobium sp. SOD10, whole genome shotgun sequence;
751285871; NZ_CCNA01000001.1
2225; Citrobacter pasteurii strain CIP 55.13, whole genome shotgun sequence;
749611130; NZ_CDHL01000044.1
2226; Cohnella kolymensis strain VKM B-2846 B2846_22, whole genome
shotgun sequence; 751596254; NZ_JXAL01000022.1
2227; Jeotgalibacillus campisalis strain SF-57 contig00001, whole genome
shotgun sequence; 751586078; NZ_JXRR01000001.1
2228; Clostridium beijerinckii strain NCIMB 14988 genome; 754484184;
NZ_CP010086.1
2229; Novosphingobium sp. P6W scaffold17, whole genome shotgun
sequence; 763097360; NZ_JXZE01000017.1
2230; Sphingomonas hengshuiensis strain WHSC-8, complete genome;
764364074; NZ_CP010836.1
2231; Sphingobium sp. YBL2, complete genome; 765344939;
NZ_CP010954.1
2232; Methanobacterium formicicum genome assembly DSM1535,
chromosome: chrI; 851114167; NZ_LN515531.1
2233; Bacillus cereus genome assembly Bacillus JRS4, contig contig000025,
whole genome shotgun sequence; 924092470; CYHM01000025.1
2234; Frankia sp. DC12 FraDC12DRAFT_scaffold1.1, whole genome
shotgun sequence; 797224947; NZ_KQ031391.1
2235; Clostridium scatologenes strain ATCC 25775, complete genome;
802929558; NZ_CP009933.1
2236; Sphingomonas sp. SRS2 contig40, whole genome shotgun sequence;
806905234; NZ_LARW01000040.1
2237; Jiangella alkaliphila strain KCTC 19222 Scaffold1, whole genome
shotgun sequence; 820820518; NZ_KQ061219.1
2238; Erythrobacter marinus strain HWDM-33 contig3, whole genome
shotgun sequence; 823659049; NZ_LBHU01000003.1
2239; Luteimonas sp. FCS-9 scf7180000000226, whole genome shotgun
sequence; 825314728; NZ_LASZ01000003.1
2240; Sphingomonas parapaucimobilis NBRC 15100 BBPI01000030, whole
genome shotgun sequence; 755134941; NZ_BBPI01000030.1
2241; Sphingobium barthaii strain KK22, whole genome shotgun sequence;
646523831; NZ_BATN01000047.1
2242; Erythrobacter marinus strain HWDM-33 contig3, whole genome
shotgun sequence; 823659049; NZ_LBHU01000003.1
2243; Streptomyces avicenniae strain NRRL B-24776 contig3.1, whole
genome shotgun sequence; 919531973; NZ_JOEK01000003.1
2244; Sphingomonas sp. Y57 scaffold74, whole genome shotgun sequence;
826051019; NZ_LDES01000074.1
2245; Xanthomonas campestris strain CFSAN033089 contig_46, whole
genome shotgun sequence; 920684790; NZ_LHBW01000046.1
2246; Croceicoccus naphthovorans strain PQ-2, complete genome;
836676868; NZ_CP011770.1
2247; Streptomyces caatingaensis strain CMAA 1322 contig09, whole genome
shotgun sequence; 906344341; NZ_LFXA01000009.1
2248; Paenibacillus sp. FJAT-27812 scaffold_0, whole genome shotgun
sequence; 922780240; NZ_LIGH01000001.1
2249; Stenotrophomonas maltophilia strain ISMMS2R, complete genome;
923060045; NZ_CP011306.1
2250; Stenotrophomonas maltophilia strain ISMMS3, complete genome;
923067758; NZ_CP011010.1
2251; Hapalosiphon sp. MRB220 contig_91, whole genome shotgun
sequence; 923076229; NZ_LIRN01000111.1
2252; Stenotrophomonas maltophilia strain B4 contig779, whole genome
shotgun sequence; 924516300; NZ_LDVR01000003.1
2253; Bacillus sp. FJAT-21352 Scaffold1, whole genome shotgun sequence;
924654439; NZ_LIUS01000003.1
2254; Sphingopyxis sp. 113P3, complete genome; 924898949; NZ_CP009452.1
2255; Sphingopyxis sp. 113P3, complete genome; 924898949; NZ_CP009452.1
2256; Streptomyces sp. CFMR 7 strain CFMR-7, complete genome;
924911621; NZ_CP011522.1
2257; Bacillus gobiensis strain FJAT-4402 chromosome; 926268043;
NZ_CP012600.1
2258; Streptomyces sp. XY431 P412contig111.1, whole genome shotgun
sequence; 926317398; NZ_LGDO01000015.1
2259; Streptomyces sp. NRRL F-6491 P443contig15.1, whole genome
shotgun sequence; 925610911; LGEE01000058.1
2260; Streptomyces sp. NRRL B-1140 P439contig15.1, whole genome
shotgun sequence; 926344107; NZ_LGEA01000058.1
2261; Streptomyces sp. NRRL B-1140 P439contig32.1, whole genome
shotgun sequence; 926344331; NZ_LGEA01000105.1
2262; Streptomyces sp. NRRL F-5755 P309contig48.1, whole genome
shotgun sequence; 926371517; NZ_LGCW01000271.1
2263; Streptomyces sp. NRRL F-5755 P309contig7.1, whole genome shotgun
sequence; 926371541; NZ_LGCW01000295.1
2264; Streptomyces sp. WM6378 P402contig63.1, whole genome shotgun
sequence; 926403453; NZ_LGDD01000321.1
2265; Streptomyces sp. WM6378 P402contig63.1, whole genome shotgun
sequence; 926403453; NZ_LGDD01000321.1
2266; Nocardia sp. NRRL S-836 P437contig39.1, whole genome shotgun
sequence; 926412104; NZ_LGDY01000113.1
2267; Paenibacillus sp. A59 contig 353, whole genome shotgun sequence;
927084730; NZ_LITU01000050.1
2268; Paenibacillus sp. A59 contig 416, whole genome shotgun sequence;
927084736; NZ_LITU01000056.1
2269; Streptomyces sp. NRRL S-444 contig322.4, whole genome shotgun
sequence; 797049078; JZWX01001028.1
2270; Altererythrobacter atlanticus strain 26DY36, complete genome;
927872504; NZ_CP011452.2
2271; Streptomyces chattanoogensis strain NRRL ISP-5002 ISP5002contig8.1,
whole genome shotgun sequence; 928897585; NZ_LGKG01000196.1
2272; Streptomyces chattanoogensis strain NRRL ISP-5002 ISP5002contig9.1,
whole genome shotgun sequence; 928897596; NZ_LGKG01000207.1
2273; Ideonella sakaiensis strain 201-F6, whole genome shotgun sequence;
928998724; NZ_BBYR01000007.1
2274; Ideonella sakaiensis strain 201-F6, whole genome shotgun sequence;
928998800; NZ_BBYR01000083.1
2275; Bacillus sp. FJAT-28004 scaffold 2, whole genome shotgun sequence;
929005248; NZ_LGHP01000003.1
2276; Novosphingobium sp. AAP1 AAP1Contigs7, whole genome shotgun
sequence; 930029075; NZ_LJHO01000007.1
2277; Novosphingobium sp. AAP1 AAP1Contigs9, whole genome shotgun
sequence; 930029077; NZ_LJHO01000009.1
2278; Actinobacteria bacterium OK074 ctg60, whole genome shotgun
sequence; 930473294; NZ_LJCV01000275.1
2279; Actinobacteria bacterium OK006 ctg112, whole genome shotgun
sequence; 930490730; NZ_LJCU01000014.1
2280; Frankia sp. R43 contig001, whole genome shotgun sequence;
937182893; NZ_LFCW01000001.1
2281; Sphingopyxis macrogoltabida strain EY-1, complete genome;
937372567; NZ_CP012700.1
2282; Xanthomonas arboricola strain CFTA 44 CITA_44_contig_26, whole
genome shotgun sequence; 937505789; NZ_LJGM01000026.1
2283; Stenotrophomonas acidaminiphila strain ZAC14D2_NAIMI4_2,
complete genome; 938883590; NZ_CP012900.1
2284; Sphingopyxis macrogoltabida strain 203, complete genome; 938956730;
NZ_CP009429.1
2285; Sphingopyxis macrogoltabida strain 203, complete genome; 938956730;
NZ_CP009429.1
2286; Sphingopyxis macrogoltabida strain 203 plasmid, complete sequence;
938956814; NZ_CP009430.1
2287; Cellulosilyticum ruminicola JCM 14822, whole genome shotgun
sequence; 938965628; NZ_BBCG01000065.1
2288; Brevundimonas sp. DS20, complete genome; 938989745; NZ_CP012897.1
2289; Brevundimonas sp. DS20, complete genome; 938989745; NZ_CP012897.1
2290; Paenibacillus sp. GD6, whole genome shotgun sequence; 939708098;
NZ_LN831198.1
2291; Paenibacillus sp. GD6, whole genome shotgun sequence; 939708105;
NZ_LN831205.1
2292; Alicyclobacillus ferrooxydans strain TC-34 contig 22, whole genome
shotgun sequence; 940346731; NZ_LJCO01000107.1
2293; Xanthomonas sp. Mitacek01 contig_17, whole genome shotgun
sequence; 941965142; NZ_LKTT01000002.1
2294; Streptomyces bingchenggensis BCW-1, complete genome; 374982757;
NC_016582.1
2295; Streptomyces pactum strain ACT12 scaffold1, whole genome shotgun
sequence; 943388237; NZ_LIQD01000001.1
2296; Streptomyces flocculus strain NRRL B-2465 B2465_contig_205, whole
genome shotgun sequence; 943674269; NZ_LIQO01000205.1
2297; Streptomyces aurantiacus strain NRRL ISP-5412 ISP-5412_contig_138,
whole genome shotgun sequence; 943881150; NZ_LIPP01000138.1
2298; Streptomyces graminilatus strain NRRL B-59124 B59124_contig_7,
whole genome shotgun sequence; 943897669; NZ_LIQQ01000007.1
2299; Streptomyces alboniger strain NRRL B-1832 B-1832_contig_37, whole
genome shotgun sequence; 943898694; NZ_LIQN01000037.1
2300; Streptomyces alboniger strain NRRL B-1832 B-1832_contig_384,
whole genome shotgun sequence; 943899498; NZ_LIQN01000384.1
2301; Streptomyces kanamyceticus strain NRRL B-2535 B-2535_contig_122,
whole genome shotgun sequence; 943922224; NZ_LIQU01000122.1
2302; Streptomyces luridiscabiei strain NRRL B-24455 B24455_contig_315,
whole genome shotgun sequence; 943927948; NZ_LIQV01000315.1
2303; Streptomyces atriruber strain NRRL B-24165 contig_124, whole
genome shotgun sequence; 943949281; NZ_LIPN01000124.1
2304; Streptomyces hirsutus strain NRRL B-2713 B2713_contig_57, whole
genome shotgun sequence; 944005810; NZ_LIQT01000057.1
2305; Streptomyces aureus strain NRRL B-2808 contig_171, whole genome
shotgun sequence; 944012845; NZ_LIPQ01000171.1
2306; Streptomyces phaeochromogenes strain NRRL B-1248 B-1248_contig_126,
whole genome shotgun sequence; 944029528; NZ_LIQZ01000126.1
2307; Streptomyces torulosus strain NRRL B-3889 B-3889_contig_18, whole
genome shotgun sequence; 944495433; NZ_LIRK01000018.1
2308; Frankia alni str. ACN14A chromosome, complete sequence;
111219505; NC_008278.1
2309; Sphingomonas sp. Leaf20 contig_1, whole genome shotgun sequence;
947349881; NZ_LMKN01000001.1
2310; Paenibacillus sp. Leaf72 contig_6, whole genome shotgun sequence;
947378267; NZ_LMLV01000032.1
2311; Sphingomonas sp. Leaf230 contig_4, whole genome shotgun sequence;
947401208; NZ_LMKW01000010.1
2312; Sanguibacter sp. Leaf3 contig_2, whole genome shotgun sequence;
947472882; NZ_LMRH01000002.1
2313; Aeromicrobium sp. Root344 contig_1, whole genome shotgun
sequence; 947552260; NZ_LMDH01000001.1
2314; Sphingopyxis sp. Root1497 contig_3, whole genome shotgun sequence;
947689975; NZ_LMGF01000003.1
2315; Sphingomonas sp. Root720 contig_7, whole genome shotgun sequence;
947704642; NZ_LMID01000015.1
2316; Sphingomonas sp. Root720 contig_8, whole genome shotgun sequence;
947704650; NZ_LMID01000016.1
2317; Sphingomonas sp. Root710 contig_1, whole genome shotgun sequence;
947721816; NZ_LMIB01000001.1
2318; Mesorhizobium sp. Root172 contig_2, whole genome shotgun sequence;
947919015; NZ_LMHP01000012.1
2319; Mesorhizobium sp. Root102 contig_3, whole genome shotgun sequence;
947937119; NZ_LMCP01000023.1
2320; Paenibacillus sp. Soil750 contig_1, whole genome shotgun sequence;
947966412; NZ_LMSD01000001.1
2321; Paenibacillus sp. Soil522 contig_3, whole genome shotgun sequence;
947983982; NZ_LMRV01000044.1
2322; Paenibacillus sp. Root52 contig_3, whole genome shotgun sequence;
948045460; NZ_LMFO01000023.1
2323; Bacillus sp. Soil768D1 contig_5, whole genome shotgun sequence;
950170460; NZ_LMTA01000046.1
2324; Paenibacillus sp. Root444D2 contig_4, whole genome shotgun
sequence; 950271971; NZ_LMEO01000034.1
2325; Paenibacillus sp. Soil766 contig_32, whole genome shotgun sequence;
950280827; NZ_LMSJ01000026.1
2326; Streptococcus pneumoniae strain type strain: N, whole genome shotgun
sequence; 950938054; NZ_CIHL01000007.1
2327; Streptomyces sp. Root1310 contig_5, whole genome shotgun sequence;
951121600; NZ_LMEQ01000031.1
2328; Bacillus muralis strain DSM 16288 Scaffold4, whole genome shotgun
sequence; 951610263; NZ_LMBV01000004.1
2329; Clostridium butyricum strain KNU-L09 chromosome 1, complete
sequence; 959868240; NZ_CP013252.1
2330; Gorillibacterium sp. SN4, whole genome shotgun sequence; 960412751;
NZ_LN881722.1
2331; Thalassobius activus strain CECT 5114, whole genome shotgun
sequence; 960424655; NZ_CYUE01000025.1
2332; Microbacterium testaceum strain NS283 contig_37, whole genome
shotgun sequence; 969836538; NZ_LDRU01000037.1
2333; Microbacterium testaceum strain NS183 contig_65, whole genome
shotgun sequence; 969919061; NZ_LDRR01000065.1
2334; Sphingopyxis sp. H050 H050_contig000006, whole genome shotgun
sequence; 970555001; NZ_LNRZ01000006.1
2335; Paenibacillus polymyxa strain KF-1 scaffold00001, whole genome
shotgun sequence; 970574347; NZ_LNZF01000001.1
2336; Luteimonas abyssi strain XH031 Scaffold1, whole genome shotgun
sequence; 970579907; NZ_KQ759763.1

TABLE 4
Exemplary Lasso Cyclase
Lasso Cyclase Peptide No: #; Species of Origin; GI#; Accession#

2337; Uncultured marine bacterium 463 clone EBAC080-L32B05 genomic
sequence; 41582259; AY458641.2
2338; Burkholderia pseudomallei strain BEF DP42.Contig323, whole genome
shotgun sequence; 686949962; JPNR01000131.1
2339; Burkholderia thailandensis E264 chromosome I, complete sequence;
83718394; NC_007651.1
2340; Frankia sp. Thr ThrDRAFT_scaffold_48.49, whole genome shotgun
sequence; 602261491; JENI01000049.1
2341; Frankia sp. Thr ThrDRAFT_scaffold_48.49, whole genome shotgun
sequence; 602261491; JENI01000049.1
2342; Sphingopyxis alaskensis RB2256, complete genome; 103485498;
NC_008048.1
2343; Sphingopyxis alaskensis RB2256, complete genome; 103485498;
NC_008048.1
2344; Streptococcus suis strain LS8I, whole genome shotgun sequence;
766595491; NZ_CEHM01000004.1
2345; Streptococcus suis SC84 complete genome, strain SC84; 253750923;
NC_012924.1
2346; Geobacter uraniireducens Rf4, complete genome; 148262085;
NC_009483.1
2347; Geobacter uraniireducens Rf4, complete genome; 148262085;
NC_009483.1
2348; Sphingomonas wittichii RW1, complete genome; 148552929;
NC_009511.1
2349; Caulobacter sp. K31, complete genome; 167643973; NC_010338.1
2350; Phenylobacterium zucineum HLK1, complete genome; 196476886;
CP000747.1
2351; Phenylobacterium zucineum HLK1, complete genome; 196476886;
CP000747.1
2352; Sanguibacter keddieii DSM 10542, complete genome; 269793358;
NC_013521.1
2353; Xylanimonas cellulosilytica DSM 15894, complete genome;
269954810; NC_013530.1
2354; Spirosoma linguale DSM 74, complete genome; 283814236;
CP001769.1
2355; Stackebrandtia nassauensis DSM 44728, complete genome; 291297538;
NC_013947.1
2356; Caulobacter segnis ATCC 21756, complete genome; 295429362;
CP002008.1
2357; Streptomyces bingchenggensis BCW-1, complete genome; 374982757;
NC_016582.1
2358; Streptomyces bingchenggensis BCW-1, complete genome; 374982757;
NC_016582.1
2359; Gallionella capsiferriformans ES-2, complete genome; 302877245;
NC_014394.1
2360; Asticcacaulis excentricus CB 48 chromosome 1, complete sequence;
315497051; NC_014816.1
2361; Burkholderia gladioli BSR3 chromosome 1, complete sequence;
327367349; CP002599.1
2362; Mycobacterium sinense strain JDM601, complete genome; 333988640;
NC_015576.1
2363; Sphingobium chlorophenolicum L-1 chromosome 1, complete
sequence; 334100279; CP002798.1
2364; Streptomyces violaceusniger Tu 4113, complete genome; 345007964;
NC_015957.1
2365; Rhodospirillum rubrum F11, complete genome; 386348020;
NC_017584.1
2366; Actinoplanes sp. SE50/110, complete genome; 386845069;
NC_017803.1
2367; Emticicia oligotrophica DSM 17448, complete genome; 408671769;
NC_018748.1
2368; Tistrella mobilis KA081020-065 plasmid pTM1, complete sequence;
442559580; NC_017957.2
2369; Bacillus thuringiensis MC28, complete genome; 407703236;
NC_018693.1
2370; Nostoc sp. PCC 7107, complete genome; 427705465; NC_019676.1
2371; Synechococcus sp. PCC 6312, complete genome; 427711179;
NC_019680.1
2372; Stanieria cyanosphaera PCC 7437, complete genome; 428267688;
CP003653.1
2373; Desulfocapsa sulfexigens DSM 10523, complete genome; 451945650;
NC_020304.1
2374; Xanthomonas citri pv. mangiferaeindicae LMG 941, whole genome
shotgun sequence; 381169556; NZ_CAHO01000002.1
2375; Streptomyces fulvissimus DSM 40593, complete genome; 488607535;
NC_021177.1
2376; Streptomyces rapamycinicus NRRL 5491 genome; 521353217;
CP006567.1
2377; Gloeobacter kilaueensis JS1, complete genome; 554634310;
NC_022600.1
2378; Kutzneria albida DSM 43870, complete genome; 754862786;
NZ_CP007155.1
2379; Mesorhizobium huakuii 7653R genome; 657121522; CP006581.1
2380; Burkholderia thailandensis E264 chromosome I, complete sequence;
83718394; NC_007651.1
2381; Sphingopyxis fribergensis strain Kp5.2, complete genome; 749188513;
NZ_CP009122.1
2382; Sphingopyxis fribergensis strain Kp5.2, complete genome; 749188513;
NZ_CP009122.1
2383; Streptomyces sp. ZJ306 hydroxylase, deacetylase, and hypothetical
proteins genes, complete cds; ikarugamycin gene cluster, complete sequence;
and GCN5-relaled N-acetyltransferase, hypothetical protein, asparagine
synthase, transcriptional regulator, ABC transporter, hypothetical proteins,
putative membrane transport protein, putative acetyltransferase, cytochrome
P450, putative alpha-glucosidase, phosphoketolase, helix-turn-helix domain-
containing protein, membrane protein, NAD-dependent epimera; 746616581;
KF954512.1
2384; Streptomyces albus strain DSM 41398, complete genome; 749658562;
NZ_CP010519.1
2385; Amycolatopsis lurida NRRL 2430, complete genome; 755908329;
CP007219.1
2386; Streptomyces lydicus A02, complete genome; 822214995; NZ_CP007699J
2387; Streptomyces lydicus A02, complete genome; 822214995; NZ_CP0076994
2388; Streptomyces lydicus A02, complete genome; 822214995; NZ_CP007699.1
2389; Streptomyces xiamenensis strain 318, complete genome; 921170702;
NZ_CP009922.2
2390; Streptomyces xiamenensis strain 318, complete genome; 921170702;
NZ_CP009922.2
2391; Uncultured bacterium clone AZ25P121 genomic sequence; 818476494;
KP274854.1
2392; Streptomyces sp. PBH53 genome; 852460626; CP011799.1
2393; Streptomyces sp. PBH53 genome; 852460626; CP011799.1
2394; Streptomyces sp. PBH53 genome; 852460626; CP011799.1
2395; Sphingopyxis sp. 113P3, complete genome; 924898949; NZ_CP009452.1
2396; Sphingopyxis sp. 113P3, complete genome; 924898949; NZ_CP009452.1
2397; Bifidobacterium longum subsp. infantis strain BT1, complete genome;
927296881; CP010411.1
2398; Nostoc piscinale CENA21 genome; 930349143; CP012036.1
2399; Citromicrobium sp. JL477, complete genome; 932136007; CP011344.1
2400; Sphingopyxis macrogoltabida strain 203, complete genome; 938956730;
NZ_CP009429.1
2401; Sphingopyxis macrogoltabida strain 203 plasmid, complete sequence;
938956814; NZ_CP009430.1
2402; Paenibacillus sp. 320-W, complete genome; 961447255; CP013653.1
2403; Streptomyces avermitilis MA-4680 = NBRC 14893, complete genome;
162960844; NC_003155.4
2404; Streptomyces avermitilis MA-4680 = NBRC 14893, complete genome;
162960844; NC_003155.4
2405; Kitasatospora setae KM-6054 DNA, complete genome; 357386972;
NC_016109.1
2406; Rhodococcus jostii lariatin biosynthetic gene cluster (larA, larB, larC,
larD, larE), complete cds; 380356103dbjAB593691.1; 0
2407; Rubrivivax gelatinosus IL144 DNA, complete genome; 383755859;
NC_017075.1
2408; Pseudomonas sp. Os17 DNA, complete genome;
771839907dbjAP014627.1; 0
2409; Pseudomonas sp. St29 DNA, complete genome;
771846103dbjAP014628.1; 0
2410; Fischerella sp. NIES-3754 DNA, complete genome;
965684975dbjAP017305.1; 0
2411; Magnetospirillum gryphiswaldense MSR-1 v2, complete genome;
568144401; NC_023065.1
2412; Magnetospirillum gryphiswaldense MSR-1 v2, complete genome;
568144401; NC_023065.1
2413; Streptococcus suis SC84 complete genome, strain SC84; 253750923;
NC_012924.1
2414; Salinibacter ruber M8 chromosome, complete genome; 294505815;
NC_014032.1
2415; Enterococcus faecalis ATCC 29212 contig24, whole genome shotgun
sequence; 401673929; ALOD01000024.1
2416; Saccharothrix espanaensis DSM 44229 complete genome; 433601838;
NC_019673.1
2417; Roseburia sp. CAG: 197 WGS project CBBL01000000 data, contig,
whole genome shotgun sequence; 524261006; CBBL010000225.1
2418; Roseburia sp. CAG: 197 WGS project CBBL01000000 data, contig,
whole genome shotgun sequence; 524261006; CBBL010000225.1
2419; Clostridium sp. CAG: 221 WGS project CBDC01000000 data, contig,
whole genome shotgun sequence; 524362382; CBDC010000065.1
2420; Clostridium sp. CAG: 411 WGS project CBIY01000000 data, contig,
whole genome shotgun sequence; 524742306; CBIY010000075.1
2421; Roseburia sp. CAG: 100 WGS project CBKV01000000 data, contig,
whole genome shotgun sequence; 524842500; CBKV010000277.1
2422; Novosphingobium sp. KN65.2 WGS project CCBH000000000 data,
contig SPHv1_Contig_228, whole genome shotgun sequence; 808402906;
CCBH010000144.1
2423; Mesorhizobium plurifarium genome assembly Mesorhizobium
plurifarium ORS1032T genome assembly, contig MPL1032_Contig_21,
whole genome shotgun sequence; 927916006; CCND01000014.1
2424; Kibdelosporangium sp. MJ 126-NF4, whole genome shotgun sequence;
754819815; NZ_CDME01000002.1
2425; Kibdelosporangium sp. MJ126-NF4 genome assembly High
quaKibdelosporangium sp. MJ126-NF4, scaffold BPA 8, whole genome
shotgun sequence; 747653426; CDME01000011.1
2426; Methanobacterium formicicum genome assembly isolate Mb9,
chromosome: I; 952971377; LN734822.1
2427; Streptococcus pneumoniae strain 37, whole genome shotgun sequence;
912648153; NZ_CKHR01000004.1
2428; Streptococcus pneumoniae genome assembly 6631_3#4, scaffold
ERS019570SCcontig000005, whole genome shotgun sequence; 879201007;
CKIK01000005.1
2429; Streptococcus pneumoniae strain type strain: N, whole genome shotgun
sequence; 950938054; NZ_CIHL01000007.1
2430; Streptococcus pneumoniae strain 37, whole genome shotgun sequence;
912648153; NZ_CKHR01000004.1
2431; Klebsiella variicola genome assembly Kv4880, contig
BN1200_Contig_75, whole genome shotgun sequence; 906292938;
CXPB01000073.1
2432; Klebsiella variicola genome assembly KvT29A, contig
BN1200_Contig_98, whole genome shotgun sequence; 906304012;
CXPA01000125.1
2433; Bacillus cereus genome assembly Bacillus JRS4, contig contig000025,
whole genome shotgun sequence; 924092470; CYHM01000025.1
2434; Achromobacter sp. 2789STDY5663426 genome assembly, contig:
ERS372662SCcontig000003, whole genome shotgun sequence; 928675838;
CYTQ01000003.1
2435; Pedobacter sp. BAL39 1103467000492, whole genome shotgun
sequence; 149277373; NZ_ABCM01000005.1
2436; Streptomyces sp. Mg1 supercont1.100, whole genome shotgun
sequence; 254387191; NZ_DS570483.1
2437; Streptomyces sviceus ATCC 29083 chromosome, whole genome
shotgun sequence; 297196766; NZ_CM000951.1
2438; Streptomyces pristinaespiralis ATCC 25486 chromosome, whole
genome shotgun sequence; 297189896; NZ_CM000950.1
2439; Enterococcus faecalis ATCC 4200 supercont1.2, whole genome shotgun
sequence; 239948580; NZ_GG670372.1
2440; Enterococcus faecalis ATCC 29212 contig24, whole genome shotgun
sequence; 401673929; ALOD01000024.1
2441; Streptomyces roseosporus NRRL 15998 supercont3.1 genomic scaffold,
whole genome shotgun sequence; 221717172; DS999644.1
2442; Streptococcus vestibularis F0396 ctg1126932565723, whole genome
shotgun sequence; 311100538; AEKO01000007.1
2443; Streptococcus vestibularis F0396 ctg1126932565723, whole genome
shotgun sequence; 311100538; AEKO01000007.1
2444; Ruminococcus albus 8 contig00035, whole genome shotgun sequence;
325680876; NZ_ADKM02000123.1
2445; Streptomyces sp. W007 contig00293, whole genome shotgun sequence;
365867746; NZ_AGSW01000272.1
2446; Streptomyces sp. W007 contig00241, whole genome shotgun sequence;
365866490; NZ_AGSW01000226.1
2447; Burkholderia pseudomallei 1258a Contig0089, whole genome shotgun
sequence; 418540998; NZ_AHJB01000089.1
2448; Burkholderia pseudomallei 1026a Contig0036, whole genome shotgun
sequence; 385360120; AHJA01000036.1
2449; Rhodanobacter sp. 115 contig437, whole genome shotgun sequence;
389759651; NZ_AJXS01000437.1
2450; Rhodanobacter thiooxydans LCS2 contig057, whole genome shotgun
sequence; 389809081; NZ_AJXW01000057.1
2451; Burkholderia thailandensis MSMB43 Scaffold3, whole genome shotgun
sequence; 424903876; NZ_JH692063.1
2452; Streptomyces auratus AGR0001 Scaffold1, whole genome shotgun
sequence; 398790069; NZ_JH725387.1
2453; Actinomyces naeslundii str. Howell 279 ctg1130888818142, whole
genome shotgun sequence; 399903251; ALJK01000024.1
2454; Enterococcus faecalis ATCC 29212 contig24, whole genome shotgun
sequence; 401673929; ALOD01000024.1
2455; Uncultured bacterium ACD_75C02634, whole genome shotgun
sequence; 406886663; AMFJ01033303.1
2456; Amycolatopsis decaplanina DSM 44594 Contig0055, whole genome
shotgun sequence; 458848256; NZ_AOHO01000055.1
2457; Streptomyces mobaraensis NBRC 13819 = DSM 40847 contig024,
whole genome shotgun sequence; 458977979; NZ_AORZ01000024.1
2458; Burkholderia pseudomallei MSHR1043 seq0003, whole genome
shotgun sequence; 469643984; AOGU01000003.1
2459; Enterococcus faecalis EnGen0363 strain RMC5 acAqY-supercont1.4,
whole genome shotgun sequence; 502232520; NZ_KB944632.1
2460; Enterococcus faecalis EnGen0233 strain UAA1014 acvJV-
supercont1.10.C18, whole genome shotgun sequence; 487281881;
AIZW01000018.1
2461; Pandoraea sp. SD6-2 scaffold29, whole genome shotgun sequence;
505733815; NZ_KB944444.1
2462; Streptomyces aurantiacus JA 4570 Seq28, whole genome shotgun
sequence; 514916412; NZ_AOPZ01000028.1
2463; Streptomyces aurantiacus JA 4570 Seq17, whole genome shotgun
sequence; 514916021; NZ_AOPZ01000017.1
2464; Enterococcus faecalis LA3B-2 Scaffold22, whole genome shotgun
sequence; 522837181; NZ_KE352807.1
2465; Paenibacillus alvei A6-6i-x PAAL66ix_14, whole genome shotgun
sequence; 528200987; ATMS01000061.1
2466; Dehalobacter sp. UNSWDHB Contig_139, whole genome shotgun
sequence; 544905305; NZ_AUUR01000139.1
2467; Actinobaculum sp. oral taxon 183 str. F0552 Scaffold15, whole genome
shotgun sequence; 545327527; NZ_KE951412.1
2468; Actinobaculum sp. oral taxon 183 str. F0552 Scaffold1, whole genome
shotgun sequence; 545327174; NZ_KE951406.1
2469; Propionibacterium acidifaciens F0233 ctg1127964738299, whole
genome shotgun sequence; 544249812; ACVN02000045.1
2470; Rubidibacter lacunae KORDI 51-2 KR51_contig00121, whole genome
shotgun sequence; 550281965; NZ_ASSJ01000070.1
2471; Rothia aeria F0184 R_aeriaHMPREF0742-1.0_Cont136.4, whole
genome shotgun sequence; 551695014; AXZG01000035.1
2472; Candidatus Halobonum tyrrellensis G22 contig00002, whole genome
shotgun sequence; 557371823; NZ_ASGZ01000002.1
2473; Streptomyces niveus NCIMB 11891 chromosome, whole genome
shotgun sequence; 566146291; NZ_CM002280.1
2474; Blastomonas sp. CACIA14H2 contig00049, whole genome shotgun
sequence; 563282524; AYSC01000019.1
2475; Frankia sp. CcI6 CcI6DRAFT_scaffold_51.52, whole genome shotgun
sequence; 563312125; AYTZ01000052.1
2476; Frankia sp. CcI6 CcI6DRAFT_scaffold_16.17, whole genome shotgun
sequence; 564016690; NZ_AYTZ01000017.1
2477; Clostridium butyricum DORA_1 Q607_CBUC00058, whole genome
shotgun sequence; 566226100; AZLX01000058.1
2478; Streptococcus sp. DORA_10 Q617_SPSC00257, whole genome
shotgun sequence; 566231608; AZMH01000257.1
2479; Candidatus Entotheonella factor TSY1_contig00913, whole genome
shotgun sequence; 575408569; AZHW01000959.1
2480; Candidatus Entotheonella gemina TSY2_contig00559, whole genome
shotgun sequence; 575423213; AZHX01000559.1
2481; Streptomyces roseosporus NRRL 11379 supercont4.1, whole genome
shotgun sequence; 588273405; NZ_ABYX02000001.1
2482; Frankia sp. Thr ThrDRAFT_scaffold_48.49, whole genome shotgun
sequence; 602261491; JENI01000049.1
2483; Frankia sp. CcI6 CcI6DRAFT_scaffold_51.52, whole genome shotgun
sequence; 563312125; AYTZ01000052.1
2484; Frankia sp. Thr ThrDRAFT_scaffold_28.29, whole genome shotgun
sequence; 602262270; JENI01000029.1
2485; Novosphingobium resinovorum strain KF1 contig000008, whole
genome shotgun sequence; 738615271; NZ_JFYZ01000008.1
2486; Novosphingobium resinovorum strain KF1 contig000008, whole
genome shotgun sequence; 738615271; NZ_JFYZ01000008.1
2487; Brevundimonas abyssalis TAR-001 DNA, contig: BAB005, whole
genome shotgun sequence; 543418148dbjBATC01000005.1; 0
2488; Bacillus akibai JCM 9157, whole genome shotgun sequence;
737696658; NZ_BAUV01000025.1
2489; Bacillus akibai JCM 9157, whole genome shotgun sequence;
737696658; NZ_BAUV01000025.1
2490; Bacillus boroniphilus JCM 21738 DNA, contig: contig_6, whole
genome shotgun sequence; 571146044dbjBAUW01000006.1; 0
2491; Bacillus sp. 17376 scaffold00002, whole genome shotgun sequence;
560433869; NZ_KI547189.1
2492; Gracilibacillus boraciitolerans JCM 21714 DNA, contig: contig_30,
whole genome shotgun sequence; 575082509dbjBAVS01000030.1; 0
2493; Gracilibacillus boraciitolerans JCM 21714 DNA, contig: contig_30,
whole genome shotgun sequence; 575082509dbjBAVS01000030.1; 0
2494; Bacterium endosymbiont of Mortierella elongata FMR23-6, whole
genome shotgun sequence; 779889750; NZ_DF850521.1
2495; Sphingopyxis sp. C-1 DNA, contig: contig_1, whole genome shotgun
sequence; 834156795dbjBBR001000001.1; 0
2496; Sphingopyxis sp. C-1 DNA, contig: contig_1, whole genome shotgun
sequence; 834156795dbjBBR001000001.1; 0
2497; Sphingopyxis sp. C-1 DNA, contig: contig_1, whole genome shotgun
sequence; 834156795dbjBBR001000001.1; 0
2498; Ideonella sakaiensis strain 201-F6, whole genome shotgun sequence;
928998724; NZ_BBYR01000007.1
2499; Brevundimonas sp. EAKA contig5, whole genome shotgun sequence;
737322991; NZ_JMQR01000005.1
2500; Streptomyces griseorubens strain JSD-1 scaffold1, whole genome
shotgun sequence; 739792456; NZ_KL503830.1
2501; Frankia sp. Thr ThrDRAFT_scaffold_28.29, whole genome shotgun
sequence; 602262270; JENI01000029.1
2502; Frankia sp. Allo2 ALLO2DRAFT_scaffold_25.26, whole genome
shotgun sequence; 737764929; NZ_JPHT01000026.1
2503; Frankia sp. CcI6 CcI6DRAFT_scaffold_16.17, whole genome shotgun
sequence; 564016690; NZ_AYTZ01000017.1
2504; Bifidobacterium reuteri DSM 23975 Contig04, whole genome shotgun
sequence; 672991374; JGZK01000004.1
2505; Streptomyces sp. JS01 contig2, whole genome shotgun sequence;
695871554; NZ_JPWW01000002.1
2506; Sphingopyxis sp. LC81 contig28, whole genome shotgun sequence;
686470905; JNFD01000021.1
2507; Sphingopyxis sp. LC81 contig24, whole genome shotgun sequence;
739659070; NZ_JNFD01000017.1
2508; Sphingopyxis sp. LC363 contig36, whole genome shotgun sequence;
739702045; NZ_JNFC01000030.1
2509; Burkholderia pseudomallei strain BEF DP42.Contig323, whole genome
shotgun sequence; 686949962; JPNR01000131.1
2510; Xanthomonas cannabis pv. phaseoli strain Nyagatare scf_52938_7,
whole genome shotgun sequence; 835885587; NZ_KN265462.1
2511; Burkholderia pseudomallei MSHR1000 scaffold1, whole genome
shotgun sequence; 740963677; NZ_KN323065.1
2512; Burkholderia pseudomallei MSHR435 Y033.Contig530, whole genome
shotgun sequence; 715120018; JRFP01000024.1
2513; Candidatus Thiomargarita nelsonii isolate Hydrate Ridge contig_1164,
whole genome shotgun sequence; 723288710; JSZA01001164.1
2514; Paenibacillus sp. P1XP2 CM49_contig000046, whole genome shotgun
sequence; 727078508; JRNV01000046.1
2515; Novosphingobium sp. P6W scaffold9, whole genome shotgun sequence;
763095630; NZ_JXZE01000009.1
2516; Streptomyces griseus strain S4-7 contig113, whole genome shotgun
sequence; 764464761; NZ_JYBE01000113.1
2517; Lechevalieria aerocolonigenes strain NRRL B-16140 contig11.3, whole
genome shotgun sequence; 772744565; NZ_JYJG01000059.1
2518; Desulfobulbaceae bacterium BRH_c16a BRHa_1001515, whole
genome shotgun sequence; 780791108; LADS01000058.1
2519; Peptococcaceae bacterium BRH_c4b BRHa_1001357, whole genome
shotgun sequence; 780813318; LADO01000010.1
2520; Peptococcaceae bacterium BRH_c4b BRHa_1001357, whole genome
shotgun sequence; 780813318; LADO01000010.1
2521; Hyphomonadaceae bacterium BRH_c29 BRHa_1005676, whole
genome shotgun sequence; 780821511; LADW01000068.1
2522; Hyphomonas sp. BRH_c22 BRHa_1001979, whole genome shotgun
sequence; 780834515; LADU01000087.1
2523; Streptomyces rubellomurinus subsp. indigofems strain ATCC 31304
contig-55, whole genome shotgun sequence; 783374270; NZ_JZKG01000056.1
2524; Streptomyces sp. NRRL S-444 contig322.4, whole genome shotgun
sequence; 797049078; JZWX01001028.1
2525; Streptomyces sp. NRRL B-1568 contig-76, whole genome shotgun
sequence; 799161588; NZ_JZWZ01000076.1
2526; Candidate division TM6 bacterium GW2011_GWF2_36_131
US03_C0013, whole genome shotgun sequence; 818310996;
LBRK01000013.1
2527; Sphingobium czechense LL01 25410_1, whole genome shotgun
sequence; 861972513; JACT01000001.1
2528; Streptomyces caatingaensis strain CMAA 1322 contig02, whole genome
shotgun sequence; 906344334; NZ_LFXA01000002.1
2529; Erythrobacter citreus LAMA 915 Contig13, whole genome shotgun
sequence; 914607448; NZ_JYNE01000028.1
2530; Paenibacillus polymyxa strain YUPP-8 scaffold32, whole genome
shotgun sequence; 924434005; LIYK01000027.1
2531; Burkholderia mallei GB8 horse 4 contig_394, whole genome shotgun
sequence; 67639376; NZ_AAHO01000116.1
2532; Streptomyces rimosus subsp. rimosus strain NRRL WC-3909
P217contig95.1, whole genome shotgun sequence; 925286515;
LGC001000284.1
2533; Streptomyces rimosus subsp. rimosus strain NRRL WC-3909
P217contig56.1, whole genome shotgun sequence; 925291008;
LGCO01000241.1
2534; Streptomyces rimosus subsp. rimosus strain NRRL WC-3869
P248contig50.1, whole genome shotgun sequence; 925315417;
LGCQ01000244.1
2535; Streptomyces rimosus subsp. rimosus strain NRRL WC-3869
P248contig20.1, whole genome shotgun sequence; 925322461;
LGCQ01000113.1
2536; Streptomyces rimosus subsp. rimosus strain NRRL WC-3898
P259contig86.1, whole genome shotgun sequence; 927279089;
NZ_LGCU01000353.1
2537; Streptomyces rimosus subsp. pseudoverticillatus strain NRRL WC-3896
P270contig8.1, whole genome shotgun sequence; 927292684;
NZ_LGCV01000415.1
2538; Streptomyces rimosus subsp. pseudoverticillatus strain NRRL WC-3896
P270contig51.1, whole genome shotgun sequence; 927292651;
NZ_LGCV01000382.1
2539; Streptomyces sp. NRRL F-5755 P309contig7.1, whole genome shotgun
sequence; 926371541; NZ_LGCW01000295.1
2540; Streptomyces sp. NRRL F-5755 P309contig50.1, whole genome
shotgun sequence; 926371520; NZ_LGCW01000274.1
2541; Streptomyces sp. NRRL F-5755 P309contig48.1, whole genome
shotgun sequence; 926371517; NZ_LGCW01000271.1
2542; Streptomyces sp. NRRL F-6492 P446contig3.1, whole genome shotgun
sequence; 926315769; NZ_LGEG01000211.1
2543; Streptomyces sp. XY332 P409contig34.1, whole genome shotgun
sequence; 927093145; NZ_LGHN01000166.1
2544; Novosphingobium sp. ST904 contig_104, whole genome shotgun
sequence; 935540718; NZ_LGJH01000063.1
2545; Actinobacteria bacterium OK006 ctg96, whole genome shotgun
sequence; 930491003; NZ_LJCU01000287.1
2546; Actinobacteria bacterium OK074 ctg60, whole genome shotgun
sequence; 930473294; NZ_LJCV01000275.1
2547; Betaproteobacteria bacterium SG8_39 WOR_8-12_2589, whole
genome shotgun sequence; 931421682; LJTQ01000030.1
2548; Candidate division BRC1 bacterium SM23_51WORSMTZ 10094,
whole genome shotgun sequence; 931536013; LJUL01000022.1
2549; Bacillus vietnamensis strain UCD-SED5 scaffold_15, whole genome
shotgun sequence; 933903534; LIXZ01000017.1
2550; Xanthomonas arboricola strain CITA 44 CITA_44_contig 26, whole
genome shotgun sequence; 937505789; NZ_LJGM01000026.1
2551; Xanthomonas sp. Mitacek01 contig 17, whole genome shotgun
sequence; 941965142; NZ_LKTT01000002.1
2552; Erythrobacteraceae bacterium HL-111 ITZY_scaf_51, whole genome
shotgun sequence; 938259025; LJSW01000006.1
2553; Halomonas sp. HL-93 ITZY_scaf_415, whole genome shotgun
sequence; 938285459; LJST01000237.1
2554; Paenibacillus sp. Soil724D2 contig 11, whole genome shotgun
sequence; 946400391; LMRY01000003.1
2555; Leucobacter sp. G161 contig50, whole genome shotgun sequence;
970293907; LOHP01000076.1
2556; Streptomyces silvensis strain ATCC 53525 53525_Assembly_Contig_22,
whole genome shotgun sequence; 970361514; LOCL01000028.1
2557; Streptococcus pneumoniae 2071004 gspj3.contig.3, whole genome
shotgun sequence; 421236283; NZ_ALBJ01000004.1
2558; Streptococcus pneumoniae 70585, complete genome; 225857809;
NC_012468.1
2559; Bacillus cereus R309803 chromosome, whole genome shotgun
sequence; 238801472; NZ_CM000720.1
2560; Bacillus cereus AH1271 chromosome, whole genome shotgun
sequence; 238801491; NZ_CM000739.1
2561; Bacillus thuringiensis serovar andalousiensis BGSC 4AW1 chromosome,
whole genome shotgun sequence; 238801506; NZ_CM000754.1
2562; Bacillus cereus VD115 supercont1.1, whole genome shotgun sequence;
423614674; NZ_JH792165.1
2563; Bacillus cereus Rock4-18 chromosome, whole genome shotgun
sequence; 238801487; NZ_CM000735.1
2564; Bacillus cereus Rock1-3 chromosome, whole genome shotgun sequence;
238801480; NZ_CM000728.1
2565; Bacillus cereus Rock3-29 chromosome, whole genome shotgun
sequence; 238801483; NZ_CM000731.1
2566; Bacillus cereus VD148 supercont1.1, whole genome shotgun sequence;
423621402; NZ_JH792156.1
2567; Bacillus thuringiensis MC28, complete genome; 407703236; NC_018693.1
2568; Bacillus cereus BAG5X2-1 supercont1.1, whole genome shotgun
sequence; 423456860; NZ_JH791975.1
2569; Bacillus cereus BAG3X2-1 supercont1.1, whole genome shotgun
sequence; 423416528; NZ_JH791923.1
2570; Bacillus cereus BAG1X1-3 supercont1.1, whole genome shotgun
sequence; 423388152; NZ_JH792182.1
2571; Escherichia coli KTE150 acwoI-supercont1.4, whole genome shotgun
sequence; 433109554; NZ_ANYF01000004.1
2572; Bacillus cereus NVH0597-99 gcontig2_1106483384196, whole genome
shotgun sequence; 196038187; NZ_ABDK02000003.1
2573; Bacillus cereus AH621 chromosome, whole genome shotgun sequence;
238801471; NZ_CM000719.1
2574; Bacillus cereus AH603 chromosome, whole genome shotgun sequence;
238801489; NZ_CM000737.1
2575; Bacillus cereus VD142 actaa-supercont2.2, whole genome shotgun
sequence; 514340871; NZ_KE150045.1
2576; Bacillus cereus BAG6O-2 supercont1.1, whole genome shotgun
sequence; 423468694; NZ_JH804628.1
2577; Bacillus cereus BtB2-4 supercont1.1, whole genome shotgun sequence;
423485377; NZ_JH804642.1
2578; Bacillus cereus HuA2-1 supercont1.1, whole genome shotgun sequence;
423508503; NZ_JH804672.1
2579; Bacillus cereus HuA4-10 supercont1.1, whole genome shotgun
sequence; 423520617; NZ_JH792148.1
2580; Bacillus cereus MC67 supercont1.2, whole genome shotgun sequence;
423557538; NZ_JH792114.1
2581; Bacillus cereus VD078 supercont1.1, whole genome shotgun sequence;
423597198; NZ_JH792251.1
2582; Bacillus cereus VD107 supercont1.1, whole genome shotgun sequence;
423609285; NZ_JH792232.1
2583; Bacillus mycoides DSM 2048 chromosome, whole genome shotgun
sequence; 238801494; NZ_CM000742.1
2584; Bacillus cereus VDM034 supercont1.1, whole genome shotgun
sequence; 423666303; NZ_JH791809.1
2585; Bacillus cereus BAG5X1-1 supercont1.1, whole genome shotgun
sequence; 423451256; NZ_JH791996.1
2586; Enterococcus faecalis ATCC 29212 contig24, whole genome shotgun
sequence; 401673929; ALOD01000024.1
2587; Enterococcus faecalis TX1341 Scfld578, whole genome shotgun
sequence; 422736691; NZ_GL457197.1
2588; Clostridium butyricum 60E.3 actYk-supercont1.1, whole genome
shotgun sequence; 488644557; NZ_KB851128.1
2589; Rhodobacter sphaeroides WS8N chromosome chrI, whole genome
shotgun sequence; 332561612; NZ_CM001161.1
2590; Microcystis aeruginosa PCC 9807, whole genome shotgun sequence;
425454132; NZ_HE973326.1
2591; Brevundimonas diminuta ATCC 11568 BDIM_scaffold00005, whole
genome shotgun sequence; 329889017; NZ_GL883086.1
2592; Brevundimonas diminuta 470-4 Scfld7, whole genome shotgun
sequence; 444405902; NZ_KB291784.1
2593; Bacillus mycoides Rock1-4 chromosome, whole genome shotgun
sequence; 238801495; NZ_CM000743.1
2594; Clostridium butyricum 5521 gcontig_1106103650482, whole genome
shotgun sequence; 182420360; NZ_ABDT01000120.2
2595; Xanthomonas citri pv. mangiferaeindicae LMG 941, whole genome
shotgun sequence; 381169556; NZ_CAHO01000002.1
2596; Xanthomonas citri pv. mangiferaeindicae LMG 941, whole genome
shotgun sequence; 381171950; NZ_CAHO01000029.1
2597; Methylosinus trichosporium OB3b MettrDRAFT_Contig106_C, whole
genome shotgun sequence; 639846426; NZ_ADVE02000001.1
2598; Streptomyces clavuligerus ATCC 27064 supercont1.55, whole genome
shotgun sequence; 254392242; NZ_DS570678.1
2599; Streptomyces rimosus subsp. rimosus strain NRRL WC-3909 P217contig95.1,
whole genome shotgun sequence; 925286515; LGCO01000284.1
2600; Streptomyces rimosus subsp. rimosus strain NRRL WC-3909 P217contig56.1,
whole genome shotgun sequence; 925291008; EGCO01000241.1
2601; Streptomyces viridochromogenes DSM 40736 supercont1.1, whole
genome shotgun sequence; 224581107; NZ_GG657757.1
2602; Streptomyces viridochromogenes DSM 40736 supercont1.1, whole
genome shotgun sequence; 224581107; NZ_GG657757.1
2603; Streptomyces viridochromogenes Tue57 Seq127, whole genome
shotgun sequence; 443625867; NZ_AMLP01000127.1
2604; Methanobacterium formicicum DSM 3637 Contig04, whole genome
shotgun sequence; 408381849; NZ_AMPO01000004.1
2605; Burkholderia pseudomallei MSHR435 Y033.Contig530, whole genome
shotgun sequence; 715120018; JRFP01000024.1
2606; Burkholderia mallei GB8 horse 4 contig_394, whole genome shotgun
sequence; 67639376; NZ_AAHO01000116.1
2607; Sphingobium yanoikuyae ATCC 51230 supercont1.1, whole genome
shotgun sequence; 427407324; NZ_JH992904.1
2608; Sphingobium yanoikuyae ATCC 51230 supercont1.1, whole genome
shotgun sequence; 427407324; NZ_JH992904.1
2609; Sphingobium yanoikuyae ATCC 51230 supercont1.1, whole genome
shotgun sequence; 427407324; NZ_JH992904.1
2610; Burkholderia pseudomallei MSHR1043 seq0003, whole genome
shotgun sequence; 469643984; AOGU01000003.1
2611; Burkholderia pseudomallei strain BEF DP42.Contig323, whole genome
shotgun sequence; 686949962; JPNR01000131.1
2612; Burkholderia pseudomallei S13 scf_1041068450778, whole genome
shotgun sequence; 254197184; NZ_CH899773.1
2613; Burkholderia pseudomallei 1026a Contig0036, whole genome shotgun
sequence; 385360120; AHJA01000036.1
2614; Burkholderia pseudomallei 305 g_contig_BUA.Contig1097, whole
genome shotgun sequence; 134282186; NZ_AAYX01000011.1
2615; Burkholderia pseudomallei 576 BUC.Contig184, whole genome
shotgun sequence; 217421258; NZ_ACCE01000004.1
2616; [Eubacterium] cellulosolvens 6 chromosome, whole genome shotgun
sequence; 389575461; NZ_CM001487.1
2617; Amycolatopsis azurea DSM 43854 contig60, whole genome shotgun
sequence; 451338568; NZ_ANMG01000060.1
2618; Xanthomonas axonopodis pv. malvacearum str. GSPB1386
1386_Scaffold6, whole genome shotgun sequence; 418516056;
NZ_AHIB01000006.1
2619; Xanthomonas citri pv. punicae str. LMG 859, whole genome shotgun
sequence; 390991205; NZ_CAGJ01000031.1
2620; Bacillus pseudomycoides DSM 12442 chromosome, whole genome
shotgun sequence; 238801497; NZ_CM000745.1
2621; Mesorhizobium amorphae CCNWGS0123 contig00204, whole genome
shotgun sequence; 357028583; NZ_AGSN01000187.1
2622; Xanthomonas gardneri ATCC 19865 XANTHO7DRAF_Contig52,
whole genome shotgun sequence; 325923334; NZ_AEQX01000392.1
2623; Xenococcus sp. PCC 7305 scaffold_00124, whole genome shotgun
sequence; 443325429; NZ_ALVZ01000124.1
2624; Leptolyngbya sp. PCC 7375 Lepto7375DRAFT_LPA.5, whole genome
shotgun sequence; 427415532; NZ_JH993797.1
2625; Streptomyces auratus AGR0001 Scaffold1, whole genome shotgun
sequence; 398790069; NZ_JH725387.1
2626; Paenibacillus dendritiformis C454 PDENDC1000064, whole genome
shotgun sequence; 374605177; NZ_AHKH01000064.1
2627; Halosimplex carlsbadense 2-9-1 contig_4, whole genome shotgun
sequence; 448406329; NZ_AOIU01000004.1
2628; Rothia aeria F0474 contig00003, whole genome shotgun sequence;
383809261; NZ_AJJQ01000036.1
2629; Paenibacillus lactis 154 ctg179, whole genome shotgun sequence;
354585485; NZ_AGIP01000020.1
2630; Fictibacillus macauensis ZFHKF-1 Contig20, whole genome shotgun
sequence; 392955666; NZ_AKKV01000020.1
2631; Marine gamma proteobacterium HTCC2148 scf_1106774214169,
whole genome shotgun sequence; 254480798; NZ_DS999224.1
2632; Paenibacillus sp. Aloe-11 GW8_15, whole genome shotgun sequence;
375307420; NZ_JH601049.1
2633; Rhodanobacter denitrificans strain 116-2 contig032, whole genome
shotgun sequence; 389798210; NZ_AJXV01000032.1
2634; Frankia saprophytica strain CN3 FrCN3DRAFT_FCB.2, whole genome
shotgun sequence; 652876473; NZ_KI912267.1
2635; Caulobacter sp. AP07 PMI01_contig_53.53, whole genome shotgun
sequence; 399069941; NZ_AKKF01000033.1
2636; Novosphingobium sp. AP12 PMI02_contig_78.78, whole genome
shotgun sequence; 399058618; NZ_AKKE01000021.1
2637; Sphingobium sp. AP49 PMI04_contig490.490, whole genome shotgun
sequence; 398386476; NZ_AJVL01000086.1
2638; Desulfosporosinus youngiae DSM 17734 chromosome, whole genome
shotgun sequence; 374578721; NZ_CM001441.1
2639; Moorea producens 3L scf52054, whole genome shotgun sequence;
332710503; NZ_GL890955.1
2640; Pedobacter sp. BAL39 1103467000500, whole genome shotgun
sequence; 149277003; NZ_ABCM01000004.1
2641; Sulfurovum sp. AR contig00449, whole genome shotgun sequence;
386284588; NZ_AJLE01000006.1
2642; Mucilaginibacter paludis DSM 18603 chromosome, whole genome
shotgun sequence; 373951708; NZ_CM001403.1
2643; Mucilaginibacter paludis DSM 18603 chromosome, whole genome
shotgun sequence; 373951708; NZ_CM001403.1
2644; Magnetospirillum caucaseum strain SO-1 contig00006, whole genome
shotgun sequence; 458904467; NZ_AONQ01000006.1
2645; Sphingomonas sp. LH128 Contig3, whole genome shotgun sequence;
402821166; NZ_ALVC01000003.1
2646; Sphingomonas sp. LH128 Contig8, whole genome shotgun sequence;
402821307; NZ_ALVC01000008.1
2647; Novosphingobium sp. Rr 2-17 contig98, whole genome shotgun
sequence; 393773868; NZ_AKFJ01000097.1
2648; Streptomyces sp. AA4 supercont1.3, whole genome shotgun sequence;
224581098; NZ_GG657748.1
2649; Moorea producens 3L scf52052, whole genome shotgun sequence;
332710285; NZ_GL890953.1
2650; Cecembia lonarensis LW9 contig000133, whole genome shotgun
sequence; 406663945; NZ_AMGM01000133.1
2651; Actinomyces sp. oral taxon 848 str. F0332 Scfld0, whole genome
shotgun sequence; 260447107; NZ_GG703879.1
2652; Actinomyces sp. oral taxon 848 str. F0332 Scfld0, whole genome
shotgun sequence; 260447107; NZ_GG703879.1
2653; Streptomyces ipomoeae 91-03 gcontig_1108499710267, whole genome
shotgun sequence; 429195484; NZ_AEJC01000118.1
2654; Frankia sp. QA3 chromosome, whole genome shotgun sequence;
392941286; NZ_CM001489.1
2655; Fischerella sp. JSC-11 ctg112, whole genome shotgun sequence;
354566316; NZ_AGIZ01000005.1
2656; Rhodobacter sp. AKP1 contigl9, whole genome shotgun sequence;
429208285; NZ_ANFS01000019.1
2657; Sphingomonas sp. SKA58 scf_1100007010440, whole genome shotgun
sequence; 211594417; NZ_CH959308.1
2658; Rubrivivax benzoatilyticus JA2 = ATCC BAA-35 strain JA2
contig_155, whole genome shotgun sequence; 332527785;
NZ_AEWG01000155.1
2659; Streptomyces clavuligerus ATCC 27064 plasmid pSCL3, whole
genome shotgun sequence; 326336949; NZ_CM001018.1
2660; Streptomyces chartreusis NRRL 12338 12338_Doro1_scaffold19,
whole genome shotgun sequence; 381200190; NZ_JH164855.1
2661; Candidatus Odyssella thessalonicensis L13 HMO_scaffold00016, whole
genome shotgun sequence; 343957487; NZ_AEWF01000005.1
2662; Candidatus Odyssella thessalonicensis L13 HMO_scaffold00016, whole
genome shotgun sequence; 343957487; NZ_AEWF01000005.1
2663; Sphingobium yanoikuyae XLDN2-5 contig000022, whole genome
shotgun sequence; 378759068; NZ_AFXE01000022.1
2664; Sphingobium yanoikuyae XLDN2-5 contig000029, whole genome
shotgun sequence; 378759075; NZ_AFXE01000029.1
2665; Paenibacillus peoriae KCTC 3763 contig9, whole genome shotgun
sequence; 389822526; NZ_AGFX01000048.1
2666; Citromicrobium sp. JLT1363 contig00009, whole genome shotgun
sequence; 341575924; NZ_AEUE01000009.1
2667; [Pseudomonas] geniculata N1 contig35, whole genome shotgun
sequence; 921165904; NZ_AJLO02000014.1
2668; Pseudomonas extremaustralis 14-3 substr. 14-3b strain 14-3
contig00001, whole genome shotgun sequence; 394743069;
NZ_AHIP01000001.1
2669; Streptomyces sp. S4, whole genome shotgun sequence; 358468594;
NZ_FR873693.1
2670; Streptomyces sp. S4, whole genome shotgun sequence; 358468601;
NZ_FR873700.1
2671; Bacillus timonensis strain MM10403188, whole genome shotgun
sequence; 403048279; NZ_HE610988.1
2672; Lunatimonas lonarensis strain AK24 S14_contig_18, whole genome
shotgun sequence; 499123840; NZ_AQHR01000021.1
2673; Mesorhizobium loti MAFF303099 DNA, complete genome; 57165207;
NC_002678.2
2674; Legionella pneumophila subsp. pneumophila ATCC 43290, complete
genome; 378775961; NC_016811.1
2675; Xanthomonas axonopodis pv. citri str. 306, complete genome;
21240774; NC_003919.1
2676; Thermobifida fusca YX, complete genome; 72160406; NC_007333.1
2677; Rhodobacter sphaeroides 2.4.1 chromosome 1, whole genome shotgun
sequence; 482849861; NZ_AKBU01000001.1
2678; Rhodospirillum rubrum F11, complete genome; 386348020; NC_017584.1
2679; Rhodospirillum rubrum F11, complete genome; 386348020; NC_017584.1
2680; Rhodospirillum rubrum F11, complete genome; 386348020; NC_017584.1
2681; Hahella chejuensis KCTC 2396, complete genome; 83642913; NC_007645.1
2682; Frankia sp. Thr ThrDRAFT_scaffold_48.49, whole genome shotgun
sequence; 602261491; JENI01000049.1
2683; Frankia sp. Thr ThrDRAFT_scaffold_28.29, whole genome shotgun
sequence; 602262270; JENI01000029.1
2684; Novosphingobium aromaticivorans DSM 12444, complete genome;
87198026; NC_007794.1
2685; Roseobacter denitrificans OCh 114, complete genome; 110677421;
NC_008209.1
2686; Frankia alni str. ACN14A chromosome, complete sequence;
111219505; NC_008278.1
2687; Pelobacter propionicus DSM 2379, complete genome; 118578449;
NC_008609.1
2688; Psychromonas ingrahamii 37, complete genome; 119943794;
NC_008709.1
2689; Rhodobacter sphaeroides ATCC 17029 chromosome 1, complete
sequence; 126460778; NC_009049.1
2690; Burkholderia pseudomallei 668 chromosome I, complete sequence;
126438353; NC_009074.1
2691; Rhodobacter sphaeroides ATCC 17025, complete genome; 146276058;
NC_009428.1
2692; Geobacter uraniireducens Rf4, complete genome; 148262085;
NC_009483.1
2693; Sulfurovum sp. NBC37-1 genomic DNA, complete genome;
152991597; NC_009663.1
2694; Acaryochloris marina MBIC11017, complete genome; 158333233;
NC_009925.1
2695; Bacillus weihenstephanensis KBAB4, complete genome; 163938013;
NC_010184.1
2696; Caulobacter sp. K31 plasmid pCAUL01, complete sequence;
167621728; NC_010335.1
2697; Caulobacter sp. K31, complete genome; 167643973; NC_010338.1
2698; Candidatus Amoebophilus asiaticus 5a2, complete genome; 189501470;
NC_010830.1
2699; Stenotrophomonas maltophilia R551-3, complete genome; 194363778;
NC_011071.1
2700; Bifidobacterium longum subsp. infantis ATCC 15697, complete
genome; 213690928; NC_011593.1
2701; Cyanothece sp. PCC 7425, complete genome; 220905643; NC_011884.1
2702; Chitinophaga pinensis DSM 2588, complete genome; 256419057;
NC_013132.1
2703; Haliangium ochraceum DSM 14365, complete genome; 262193326;
NC_013440.1
2704; Rhodothermus marinus DSM 4252, complete genome; 268315578;
NC_013501.1
2705; Thermobaculum terrenum ATCC BAA-798 chromosome 1, complete
sequence; 269925123; NC_013525.1
2706; Thermobaculum terrenum ATCC BAA-798 chromosome 2, complete
sequence; 269838913; NC_013526.1
2707; Thermobaculum terrenum ATCC BAA-798 chromosome 2, complete
sequence; 269838913; NC_013526.1
2708; Sphingobium japonicum UT26S DNA, chromosome 1, complete
genome; 294009986; NC_014006.1
2709; Sphingobium japonicum UT26S plasmid pCHQ1 DNA, complete
genome; 294023656; NC_014007.1
2710; Salinibacter ruber M8 chromosome, complete genome; 294505815;
NC_014032.1
2711; Salinibacter ruber M8 chromosome, complete genome; 294505815;
NC_014032.1
2712; Legionella pneumophila 2300/99 Alcoy, complete genome; 296105497;
NC_014125.1
2713; Nocardiopsis dassonvillei subsp. dassonvillei DSM 43111 chromosome
1, complete sequence; 297558985; NC_014210.1
2714; Amycolatopsis mediterranei S699, complete genome; 384145136;
NC_017186.1
2715; Butyrivibrio proteoclasticus B316 chromosome 1, complete sequence;
302669374; NC_014387.1
2716; Paenibacillus polymyxa E681, complete genome; 864439741;
NC_014483.2
2717; Paenibacillus polymyxa M1 main chromosome, complete genome;
386038690; NC_017542.1
2718; Leadbetterella byssophila DSM 17132, complete genome; 312128809;
NC_014655.1
2719; Frankia inefficax, complete genome; 312193897; NC_014666.1
2720; Frankia inefficax, complete genome; 312193897; NC_014666.1
2721; Burkholderia rhizoxinica HKI 454, complete genome; 312794749;
NC_014722.1
2722; Burkholderia rhizoxinica HKI 454, complete genome; 312794749;
NC_014722.1
2723; Asticcacaulis excentricus CB 48 chromosome 2, complete sequence;
315499382; NC_014817.1
2724; Terriglobus saanensis SP1PR4, complete genome; 320105246;
NC_014963.1
2725; Syntrophobotulus glycolicus DSM 8271, complete genome; 325288201;
NC_015172.1
2726; Methanobacterium lacus strain AL-21, complete genome; 325957759;
NC_015216.1
2727; Marinomonas mediterranea MMB-1, complete genome; 326793322;
NC_015276.1
2728; Desulfobacca acetoxidans DSM 11109, complete genome; 328951746;
NC_015388.1
2729; Methylomonas methanica MC09, complete genome; 333981747;
NC_015572.1
2730; Methylomonas methanica MC09, complete genome; 333981747;
NC_015572.1
2731; Methanobacterium paludis strain SWAN1, complete genome;
333986242; NC_015574.1
2732; Novosphingobium sp. PP1Y Lpl large plasmid, complete replicon;
334133217; NC_015579.1
2733; Novosphingobium sp. PP1Y main chromosome, complete replicon;
334139601; NC_015580.1
2734; Frankia symbiont of Datisca glomerata, complete genome; 336176139;
NC_015656.1
2735; Halopiger xanaduensis SH-6 plasmid pHALXA01, complete genome;
336251750; NC_015658.1
2736; Mesorhizobium opportunistum WSM2075, complete genome;
337264537; NC_015675.1
2737; Runella slithyformis DSM 19594, complete genome; 338209545;
NC_015703.1
2738; Runella slithyformis DSM 19594, complete genome; 338209545;
NC_015703.1
2739; Roseobacter litoralis Och 149, complete genome; 339501577;
NC_015730.1
2740; Streptomyces violaceusniger Tu 4113 plasmid pSTRVI01, complete
sequence; 345007457; NC_015951.1
2741; Rhodothermus marinus SG0.5JP17-172, complete genome; 345301888;
NC_015966.1
2742; Sphingobium sp. SYK-6 DNA, complete genome; 347526385;
NC_015976.1
2743; Sphingobium sp. SYK-6 DNA, complete genome; 347526385;
NC_015976.1
2744; Chloracidobacterium thermophilum B chromosome 1, complete
sequence; 347753732; NC_016024.1
2745; Kitasatospora setae KM-6054 DNA, complete genome; 357386972;
NC_016109.1
2746; Kitasatospora setae KM-6054 DNA, complete genome; 357386972;
NC_016109.1
2747; Streptomyces cattleya str. NRRL 8057 main chromosome, complete
genome; 357397620; NC_016111.1
2748; Desulfosporosinus orientis DSM 765, complete genome; 374992780;
NC_016584.1
2749; Paenibacillus terrae HPL-003, complete genome; 374319880;
NC_016641.1
2750; Bacillus megaterium WSH-002, complete genome; 384044176;
NC_017138.1
2751; Francisella cf. novicida 3523, complete genome; 387823583;
NC_017449.1
2752; Streptococcus salivarius JIM8777 complete genome; 387783149;
NC_017595.1
2753; Tistrella mobilis KA081020-065, complete genome; 389875858;
NC_017956.1
2754; Tistrella mobilis KA081020-065 plasmid pTM3, complete sequence;
389874236; NC_017958.1
2755; Legionella pneumophila subsp. pneumophila str. Lorraine chromosome,
complete genome; 397662556; NC_018139.1
2756; Nocardiopsis alba ATCC BAA-2165, complete genome; 403507510;
NC_018524.1
2757; Streptomyces venezuelae ATCC 10712 complete genome; 408675720;
NC_018750.1
2758; Saccharothrix espanaensis DSM 44229 complete genome; 433601838;
NC_019673.1
2759; Nostoc sp. PCC 7107, complete genome; 427705465; NC_019676.1
2760; Rivularia sp. PCC 7116, complete genome; 427733619; NC_019678.1
2761; Rivularia sp. PCC 7116, complete genome; 427733619; NC_019678.1
2762; Synechococcus sp. PCC 6312, complete genome; 427711179;
NC_019680.1
2763; Nostoc sp. PCC 7524, complete genome; 427727289; NC_019684.1
2764; Calothrix sp. PCC 6303, complete genome; 428296779; NC_019751.1
2765; Crinalium epipsammum PCC 9333, complete genome; 428303693;
NC_019753.1
2766; Cylindrospermum stagnate PCC 7417, complete genome; 434402184;
NC_019757.1
2767; Thermobacillus composti KWC4, complete genome; 430748349;
NC_019897.1
2768; Mesorhizobium australicum WSM2073, complete genome; 433771415;
NC_019973.1
2769; Rhodanobacter denitrificans strain 2APBS1, complete genome;
469816339; NC_020541.1
2770; Bacillus sp. 1NLA3E, complete genome; 488570484; NC_021171.1
2771; Bacillus sp. 1NLA3E, complete genome; 488570484; NC_021171.1
2772; Burkholderia thailandensis MSMB121 chromosome 1, complete
sequence; 488601775; NC_021173.1
2773; Streptomyces davawensis strain JCM 4913 complete genome;
471319476; NC_020504.1
2774; Streptomyces davawensis strain JCM 4913 complete genome;
471319476; NC_020504.1
2775; Desulfotomaculum acetoxidans DSM 771, complete genome;
258513366; NC_013216.1
2776; Desulfotomaculum acetoxidans DSM 771, complete genome;
258513366; NC_013216.1
2777; Actinosynnema mirum DSM 43827, complete genome; 256374160;
NC_013093.1
2778; Actinosynnema mirum DSM 43827, complete genome; 256374160;
NC_013093.1
2779; Rhodobacter sphaeroides KD131 chromosome 1, complete sequence;
221638099; NC_011963.1
2780; Bacillus cereus BAG2O-3 acfXF-supercont1.1, whole genome shotgun
sequence; 507017505; NZ_KB976530.1
2781; Bacillus cereus HuA2-9 acqVt-supercont1.1, whole genome shotgun
sequence; 507020427; NZ_KB976152.1
2782; Bacillus cereus HuA3-9 acqVv-supercont1.4, whole genome shotgun
sequence; 507024338; NZ_KB976146.1
2783; Bacillus cereus VD118 acrHo-supercont1.9, whole genome shotgun
sequence; 507035131; NZ_KB976800.1
2784; Bacillus cereus VD131 acrHi-supercont1.9, whole genome shotgun
sequence; 507037581; NZ_KB976660.1
2785; Bacillus cercus VD136 acrHc-supercont1.1, whole genome shotgun
sequence; 507041177; NZ_KB976717.1
2786; Bacillus cereus VDM019 achrj-supercont1.2, whole genome shotgun
sequence; 507056808; NZ_KB976199.1
2787; Bacillus cereus VDM053 acrGS-supcrcont1.7, whole genome shotgun
sequence; 507060152; NZ_KB976714.1
2788; Bacillus cereus VDM006 acrHb-supercont1.1, whole genome shotgun
sequence; 507060269; NZ_KB976864.1
2789; Bacillus cereus VDM021 acrHe-supercont1.1, whole genome shotgun
sequence; 507061629; NZ_KB976905.1
2790; Thermobifida fusca TM51 contig028, whole genome shotgun sequence;
510814910; NZ_AOSG01000028.1
2791; Halomonas anticariensis FP35 = DSM 16096 strain FP35 Scaffold1,
whole genome shotgun sequence; 514429123; NZ_KE332377.1
2792; Halomonas anticariensis FP35 = DSM 16096 strain FP35 Scaffold1,
whole genome shotgun sequence; 514429123; NZ_KE332377.1
2793; Halomonas anticariensis FP35 = DSM 16096 strain FP35 Scaffold1,
whole genome shotgun sequence; 514429123; NZ_KE332377.1
2794; Streptomyces sp. HPH0547 aczHZ-supercont1.2, whole genome
shotgun sequence; 512676856; NZ_KE150472.1
2795; Acinetobacter gyllenbergii MTCC 11365 contig1, whole genome
shotgun sequence; 514348304; NZ_ASQH01000001.1
2796; Streptomyces aurantiacus JA 4570 Seq63, whole genome shotgun
sequence; 514917321; NZ_AOPZ01000063.1
2797; Streptomyces aurantiacus JA 4570 Seq109, whole genome shotgun
sequence; 514918665; NZ_AOPZ01000109.1
2798; Actinoalloteichus spitiensis RMV-1378 Contig406, whole genome
shotgun sequence; 483112234; NZ_AGVX02000406.1
2799; Paenibacillus polymyxa OSY-DF Contig136, whole genome shotgun
sequence; 484036841; NZ_AIPP01000136.1
2800; Fischerella muscicola SAG 1427-1 = PCC 73103 contig00215, whole
genome shotgun sequence; 484073367; NZ_AJLJ01000207.1
2801; Fischerella muscicola PCC 7414 contig00109, whole genome shotgun
sequence; 484075173; NZ_AJLK01000109.1
2802; Fischerella muscicola PCC 7414 contig00153, whole genome shotgun
sequence; 484075372; NZ_AJLK01000153.1
2803; Fischerella thermalis PCC 7521 contig00099, whole genome shotgun
sequence; 484076371; NZ_AJLL01000098.1
2804; Xanthomonas arboricola pv. juglandis str. NCPPB 1447 contig00105,
whole genome shotgun sequence; 484083029; NZ_AJTL01000105.1
2805; Sphingobium xenophagum QYY contig015, whole genome shotgun
sequence; 484272664; NZ_AKIB01000015.1
2806; Pedobacter arcticus A12 Scaffold2, whole genome shotgun sequence;
484345004; NZ_JH947126.1
2807; Leptolyngbya boryana PCC 6306 LepboDRAFT_LPC.1, whole
genome shotgun sequence; 482909028; NZ_KB731324.1
2808; Spirulina subsalsa PCC 9445 Contig210, whole genome shotgun
sequence; 482909235; NZ_JH980292.1
2809; Fischerella sp. PCC 9339 PCC9339DRAFT_scaffold1.1, whole genome
shotgun sequence; 482909394; NZ_JH992898.1
2810; Mastigocladopsis repens PCC 10914 Mas10914DRAFT_scaffold1.1,
whole genome shotgun sequence; 482909462; NZ_JH992901.1
2811; Methylococcus capsulatus str. Texas = ATCC 19069 strain Texas
contig0129, whole genome shotgun sequence; 483090991;
NZ_AMCE01000064.1
2812; Lactococcus garvieae Tac2 Tac2Contig_33, whole genome shotgun
sequence; 483258918; NZ_AMFE01000033.1
2813; Paenisporosarcina sp. TG-14 111.TG14.1_1, whole genome shotgun
sequence; 483299154; NZ_AMGD01000001.1
2814; Paenibacillus sp. ICGEB2008 Contig_7, whole genome shotgun
sequence; 483624383; NZ_AMQU01000007.1
2815; Amphibacillus jilinensis Y1 Scaffold2, whole genome shotgun
sequence; 483992405; NZ_JH976435.1
2816; Alpha proteobacterium LLX12A LLX12A_contig00014, whole genome
shotgun sequence; 483996931; NZ_AMYX01000014.1
2817; Alpha proteobacterium LLX12A LLX12A_contig00026, whole genome
shotgun sequence; 483996974; NZ_AMYX01000026.1
2818; Alpha proteobacterium LLX12A LLX12A_contig00084, whole genome
shotgun sequence; 483997176; NZ_AMYX01000084.1
2819; Alpha proteobacterium L41A L41A_contig00002, whole genome
shotgun sequence; 483997957; NZ_AMYY01000002.1
2820; Nocardiopsis alba DSM 43377 contig_10, whole genome shotgun
sequence; 484007121; NZ_ANAC01000010.1
2821; Nocardiopsis sp. TP-A0876 strain NBRC 110039, whole genome
shotgun sequence; 754924215; NZ_BAZE01000001.1
2822; Nocardiopsis halophila DSM 44494 contig_138, whole genome shotgun
sequence; 484007841; NZ_ANAD01000138.1
2823; Nocardiopsis halophila DSM 44494 contig_138, whole genome shotgun
sequence; 484007841; NZ_ANAD01000138.1
2824; Nocardiopsis halophila DSM 44494 contig_197, whole genome shotgun
sequence; 484008051; NZ_ANAD01000197.1
2825; Nocardiopsis baichengensis YIM 90130 Scaffold15_1, whole genome
shotgun sequence; 484012558; NZ_ANAS01000033.1
2826; Nocardiopsis halotolerans DSM 44410 contig_26, whole genome
shotgun sequence; 484015294; NZ_ANAX01000026.1
2827; Nocardiopsis kunsanensis DSM 44524 contig_3, whole genome shotgun
sequence; 484016825; NZ_ANAY01000003.1
2828; Nocardiopsis kunsanensis DSM 44524 contig_16, whole genome
shotgun sequence; 484016872; NZ_ANAY01000016.1
2829; Nocardiopsis potens DSM 45234 contig_25, whole genome shotgun
sequence; 484017897; NZ_ANBB01000025.1
2830; Nocardiopsis lucentensis DSM 44048 contig_935, whole genome
shotgun sequence; 484021665; NZ_ANBC01000935.1
2831; Nocardiopsis alkaliphila YIM 80379 contig_111, whole genome
shotgun sequence; 484022237; NZ_ANBD01000111.1
2832; Nocardiopsis salina YIM 90010 contig_87, whole genome shotgun
sequence; 484023389; NZ_ANBF01000087.1
2833; Nocardiopsis salina YIM 90010 contig_204, whole genome shotgun
sequence; 484023808; NZ_ANBF01000204.1
2834; Nocardiopsis chromatogenes YIM 90109 contig_59, whole genome
shotgun sequence; 484026076; NZ_ANBH01000059.1
2835; Porphyrobacter sp. AAP82 Contig35, whole genome shotgun sequence;
484033307; NZ_ANFX01000035.1
2836; Blastomonas sp. AAP53 Contig8, whole genome shotgun sequence;
484033611; NZ_ANFZ01000008.1
2837; Blastomonas sp. AAP53 Contig14, whole genome shotgun sequence;
484033631; NZ_ANFZ01000014.1
2838; Paenibacillus sp. PAMC 26794 5104_29, whole genome shotgun
sequence; 484070054; NZ_ANHX01000029.1
2839; Oscillatoria sp. PCC 10802 Osc10802DRAFT_Contig7.7, whole
genome shotgun sequence; 484104632; NZ_KB235948.1
2840; Oscillatoria sp. PCC 10802 Osc10802DRAFT_Contig7.7, whole
genome shotgun sequence; 484104632; NZ_KB235948.1
2841; Clostridium botulinum CB11/1-1 CB_contig00105, whole genome
shotgun sequence; 484141779; NZ_AORM01000006.1
2842; Actinopolyspora halophila DSM 43834 ActhaDRAFT_contig1.1_C,
whole genome shotgun sequence; 484203522; NZ_AQUI01000002.1
2843; Asticcacaulis benevestitus DSM 16100 = ATCC BAA-896 strain DSM
16100 B060DRAFT_scaffold_12.13_C, whole genome shotgun sequence;
484226753; NZ_AQWM01000013.1
2844; Asticcacaulis benevestitus DSM 16100 = ATCC BAA-896 strain DSM
16100 B060DRAFT_scaffold_31.32_C, whole genome shotgun sequence;
484226810; NZ_AQWM01000032.1
2845; Streptomyces sp. FxanaC1 B074DRAFT_scaffold_1.2_C, whole
genome shotgun sequence; 484227180; NZ_AQWO01000002.1
2846; Streptomyces sp. FxanaC1 B074DRAFT_scaffold_7.8_C, whole
genome shotgun sequence; 484227195; NZ_AQWO01000008.1
2847; Smaragdicoccus niigatensis DSM 44881 = NBRC 103563 strain DSM
44881 F600DRAFT_scaffold00011.11_C, whole genome shotgun sequence;
484234624; NZ_AQXZ01000009.1
2848; Sphingomonas melonis DAPP-PG 224 Sphme3DRAFT_scaffold1.1,
whole genome shotgun sequence; 482984722; NZ_KB900605.1
2849; Verrucomicrobium sp. 3C A37ADRAFT_scaffold1.1, whole genome
shotgun sequence; 483219562; NZ_KB901875.1
2850; Verrucomicrobium sp. 3C A37ADRAFT_scaffold1.1, whole genome
shotgun sequence; 483219562; NZ_KB901875.1
2851; Bradyrhizobium sp. WSM2793 A3ASDRAFT_scaffold_24.25, whole
genome shotgun sequence; 483314733; NZ_KB902785.1
2852; Streptomyces vitaminophilus DSM 41686 A3IGDRAFT_scaffold_10.11,
whole genome shotgun sequence; 483682977; NZ_KB904636.1
2853; Ancylobacter sp. FA202 A3M1DRAFT_scaffold1.1, whole genome
shotgun sequence; 483720774; NZ_KB904818.1
2854; Filamentous cyanobacterium ESFC-1 A3MYDRAFT_scaffold1.1,
whole genome shotgun sequence; 483724571; NZ_KB904821.1
2855; Streptomyces sp. CcalMP-8W B053DRAFT_scaffold_17.18, whole
genome shotgun sequence; 483961830; NZ_KB890924.1
2856; Streptomyces sp. ScaeMP-e10 B061DRAFT_scaffold_0.1, whole
genome shotgun sequence; 483967534; NZ_KB891296.1
2857; Streptomyces sp. KhCrAH-244 B069DRAFT_scaffold_11.12, whole
genome shotgun sequence; 483969755; NZ_KB891596.1
2858; Streptomyces sp. HmicA12 B072DRAFT_scaffold_19.20, whole
genome shotgun sequence; 483972948; NZ_KB891808.1
2859; Streptomyces sp. MspMP-M5 B073DRAFT_scaffold_27.28, whole
genome shotgun sequence; 483974021; NZ_KB891893.1
2860; Arthrobacter sp. 161MFSha2.1 C567DRAFT_scaffold00006.6, whole
genome shotgun sequence; 484021228; NZ_KB895788.1
2861; Streptomyces sp. CNY228 D330DRAFT_scaffold00011.11, whole
genome shotgun sequence; 484057944; NZ_KB898231.1
2862; Streptomyces sp. CNB091 D581DRAFT_scaffold00010.10, whole
genome shotgun sequence; 484070161; NZ_KB898999.1
2863; Sphingobium xenophagum NBRC 107872, whole genome shotgun
sequence; 483527356; NZ_BARE01000016.1
2864; Streptomyces sp. TOR3209 Contig612, whole genome shotgun
sequence; 484867900; NZ_AGNH01000612.1
2865; Streptomyces sp. TOR3209 Contig613, whole genome shotgun
sequence; 484867902; NZ_AGNH01000613.1
2866; Stenotrophomonas maltophilia RR-10 STMALcontig40, whole genome
shotgun sequence; 484978121; NZ_AGRB01000040.1
2867; Bacillus oceanisediminis 2691 contig2644, whole genome shotgun
sequence; 485048843; NZ_ALEG01000067.1
2868; Calothrix sp. PCC 7103 Cal7103DRAFT_CPM.6, whole genome
shotgun sequence; 485067373; NZ_KB217478.1
2869; Pseudanabaena sp. PCC 6802 Pse6802_scaffold_5, whole genome
shotgun sequence; 485067426; NZ_KB235914.1
2870; Actinomadura atramentaria DSM 43919 strain SF2197
G339DRAFT_scaffold00002.2, whole genome shotgun sequence; 485090585;
NZ_KB907209.1
2871; Novispirillum itersonii subsp. itersonii ATCC 12639
G365DRAFT_scaffold00001.1, whole genome shotgun sequence; 485091510;
NZ_KB907337.1
2872; Novispirillum itersonii subsp. itersonii ATCC 12639
G365DRAFT_scaffold00001.1, whole genome shotgun sequence; 485091510;
NZ_KB907337.1
2873; Paenibacillus polymyxa ATCC 842 PPt02_scaffold1, whole genome
shotgun sequence; 485269841; NZ_GL905390.1
2874; Actinopolyspora mortivallis DSM 44261 strain HS-1
ActmoDRAFT_scaffold1.1, whole genome shotgun sequence; 486324513;
NZ_KB913024.1
2875; Mesorhizobium loti NZP2037 Meslo3DRAFT_scaffold1.1, whole
genome shotgun sequence; 486325193; NZ_KB913026.1
2876; Paenibacillus sp. HW567 B212DRAFT_scaffold1.1, whole genome
shotgun sequence; 486346141; NZ_KB910518.1
2877; Bacillus sp. 123MFChir2 H280DRAFT_scaffold00030.30, whole
genome shotgun sequence; 487368297; NZ_KB910953.1
2878; Streptomyces canus 299MFChir4.1 H293DRAFT_scaffold00032.32,
whole genome shotgun sequence; 487385965; NZ_KB911613.1
2879; Kribbella catacumbae DSM 19601 A3ESDRAFT_scaffold_7.8_C,
whole genome shotgun sequence; 484207511; NZ_AQUZ01000008.1
2880; Paenibacillus riograndensis SBR5 Contig78, whole genome shotgun
sequence; 485470216; NZ_A
2881; Lamprocystis purpurea DSM 4197 A39ODRAFT_scaffold_0.1, whole
genome shotgun sequence; 483254584; NZ_KB902362.1
2882; Nonomuraea coxensis DSM 45129 A3G7DRAFT_scaffold_4.5, whole
genome shotgun sequence; 483454700; NZ_KB903974.1
2883; Streptomyces scabnsporus DSM 41855 A3ICDRAFT_scaffold_0.1,
whole genome shotgun sequence; 483624586; NZ_KB889561.1
2884; Amycolatopsis alba DSM 44262 scaffold1, whole genome shotgun
sequence; 486330103; NZ_KB913032.1
2885; Amycolatopsis benzoatilytica AK 16/65 AmybeDRAFT_scaffold1.1,
whole genome shotgun sequence; 486399859; NZ_KB912942.1
2886; Amycolatopsis nigrescens CSC17Ta-90 AmyniDRAFT_Contig68.1_C,
whole genome shotgun sequence; 487404592; NZ_ARVW01000001.1
2887; Amycolatopsis nigrescens CSC17Ta-90 AmyniDRAFT_Contig68.1_C,
whole genome shotgun sequence; 487404592; NZ_ARVW01000001.1
2888; Amycolatopsis nigrescens CSC17Ta-90 AmyniDRAFT_Contig68.1_C,
whole genome shotgun sequence; 487404592; NZ_ARVW01000001.1
2889; Reyranella massiliensis 521, whole genome shotgun sequence;
484038067; NZ_HE997181.1
2890; Acidobacteriaceae bacterium KBS 83 G002DRAFT_scaffold00007.7,
whole genome shotgun sequence; 485076323; NZ_KB906739.1
2891; Sphingobium lactosutens DS20 contig 107, whole genome shotgun
sequence; 544811486; NZ_ATDP01000107.1
2892; Novosphingobium lindaniclasticum LE124 contig147, whole genome
shotgun sequence; 544819688; NZ_ATHL01000147.1
2893; Actinobaculum sp. oral taxon 183 str. F0552 Scaffold15, whole genome
shotgun sequence; 545327527; NZ_KE951412.1
2894; Novosphingobium sp. B-7 scaffold147, whole genome shotgun
sequence; 514419386; NZ_KE148338.1
2895; Sphingomonas-like bacterium B12, whole genome shotgun sequence;
484113405; NZ_BACX01000237.1
2896; Sphingomonas-like bacterium B12, whole genome shotgun sequence;
484113491; NZ_BACX01000258.1
2897; Thermoactinomyces vulgaris strain NRRL F-5595 F5595contig15.1,
whole genome shotgun sequence; 929862756; NZ_LGKI01000090.1
2898; Clostridium saccharobutylicum DSM 13864, complete genome;
550916528; NC_022571.1
2899; Butyrivibrio fibrisolvens AB2020 G616DRAFT_scaffold00015.15_C,
whole genome shotgun sequence; 551012921; NZ_ATVZ01000015.1
2900; Butyrivibrio sp. XPD2006 G590DRAFT_scaffold00008.8_C, whole
genome shotgun sequence; 551021553; NZ_ATVT01000008.1
2901; Butyrivibrio sp. AE3009 G588DRAFT_scaffold00030.30_C, whole
genome shotgun sequence; 551035505; NZ_ATVS01000030.1
2902; Acidobacteriaceae bacterium TAA166 strain TAA 166
H979DRAFT_scaffold_0.1_C, whole genome shotgun sequence; 551216990;
NZ_ATWD01000001.1
2903; Acidobacteriaceae bacterium TAA166 strain TAA 166
H979DRAFT_scaffold_0.1_C, whole genome shotgun sequence; 551216990;
NZ_ATWD01000001.1
2904; Acidobacteriaceae bacterium TAA166 strain TAA 166
H979DRAFT_scaffold_0.1_C, whole genome shotgun sequence; 551216990;
NZ_ATWD01000001.1
2905; Leptolyngbya sp. Heron Island J 50, whole genome shotgun sequence;
553739852; NZ_AWNH01000066.1
2906; Leptolyngbya sp. Heron Island J 50, whole genome shotgun sequence;
553739852; NZ_AWNH01000066.1
2907; Leptolyngbya sp. Heron Island J 67, whole genome shotgun sequence;
553740975; NZ_AWNH01000084.1
2908; Klebsiella pneumoniae BIDMC 22 addSE-supercont1.4, whole genome
shotgun sequence; 556268595; NZ_KI535436.1
2909; Klebsiella pneumoniae MGH 19 addTc-supercont1.2, whole genome
shotgun sequence; 556494858; NZ_KI535678.1
2910; Asticcacaulis sp. AC466 contig00008, whole genome shotgun sequence;
557833377; NZ_AWGE01000008.1
2911; Asticcacaulis sp. AC466 contig00033, whole genome shotgun sequence;
557835508; NZ_AWGE01000033.1
2912; Asticcacaulis sp. YBE204 contig00005, whole genome shotgun
sequence; 557839256; NZ_AWGF01000005.1
2913; Asticcacaulis sp. YBE204 contig00010, whole genome shotgun
sequence; 557839714; NZ_AWGF01000010.1
2914; Streptomyces roseochromogenus subsp. oscitans DS 12.976
chromosome, whole genome shotgun sequence; 566155502;
NZ_CM002285.1
2915; Streptomyces roseochromogenus subsp. oscitans DS 12.976
chromosome, whole genome shotgun sequence; 566155502;
NZ_CM002285.1
2916; Bacillus sp. 17376 scaffold00002, whole genome shotgun sequence;
560433869; NZ_KI547189.1
2917; Mesorhizobium sp. LSJC285A00 scaffold0007, whole genome shotgun
sequence; 563442031; NZ_AYVK01000007.1
2918; Mesorhizobium sp. LSJC277A00 scaffold0014, whole genome shotgun
sequence; 563459186; NZ_AYVM01000014.1
2919; Mesorhizobium sp. LSJC269B00 scaffold0015, whole genome shotgun
sequence; 563464990; NZ_AYVN01000015.1
2920; Mesorhizobium sp. LSJC268A00 scaffold0012, whole genome shotgun
sequence; 563469252; NZ_AYVO01000012.1
2921; Mesorhizobium sp. LSJC265A00 scaffold0015, whole genome shotgun
sequence; 563472037; NZ_AYVP01000015.1
2922; Mesorhizobium sp. LSJC264A00 scaffold0029, whole genome shotgun
sequence; 563478461; NZ_AYVQ01000029.1
2923; Mesorhizobium sp. LSJC255A00 scaffold0001, whole genome shotgun
sequence; 563480247; NZ_AYVR01000001.1
2924; Mesorhizobium sp. LSHC426A00 scaffold0005, whole genome shotgun
sequence; 563492715; NZ_AYWO1000005.1
2925; Mesorhizobium sp. LSHC422A00 scaffold0012, whole genome shotgun
sequence; 563497640; NZ_AYVX01000012.1
2926; Mesorhizobium sp. LNJC405B00 scaffold0005, whole genome shotgun
sequence; 563523441; NZ_AYWC01000005.1
2927; Mesorhizobium sp. LNJC403B00 scaffold0001, whole genome shotgun
sequence; 563526426; NZ_AYWD01000001.1
2928; Mesorhizobium sp. LNJC399B00 scaffold0004, whole genome shotgun
sequence; 563530011; NZ_AYWE01000004.1
2929; Mesorhizobium sp. LNJC398B00 scaffold0002, whole genome shotgun
sequence; 563532486; NZ_AYWF01000002.1
2930; Mesorhizobium sp. LNJC395A00 scaffold0011, whole genome shotgun
sequence; 563536456; NZ_AYWG01000011.1
2931; Mesorhizobium sp. LNJC394B00 scaffold0005, whole genome shotgun
sequence; 563539234; NZ_AYWH01000005.1
2932; Mesorhizobium sp. LNJC384A00 scaffold0009, whole genome shotgun
sequence; 563544477; NZ_AYWK01000009.1
2933; Mesorhizobium sp. LNJC380A00 scaffold0009, whole genome shotgun
sequence; 563546593; NZ_AYWL01000009.1
2934; Mesorhizobium sp. LNHC232B00 scaffold0020, whole genome
shotgun sequence; 563561985; NZ_AYWP01000020.1
2935; Mesorhizobium sp. LNHC229A00 scaffold0006, whole genome
shotgun sequence; 563567190; NZ_AYWQ01000006.1
2936; Mesorhizobium sp. LNHC221B00 scaffold0001, whole genome
shotgun sequence; 563570867; NZ_AYWR01000001.1
2937; Mesorhizobium sp. LNHC220B00 scaffold0002, whole genome
shotgun sequence; 563576979; NZ_AYWS01000002.1
2938; Mesorhizobium sp. LNHC209A00 scaffold0002, whole genome
shotgun sequence; 563784877; NZ_AYWT01000002.1
2939; Mesorhizobium sp. L48C026A00 scaffold0030, whole genome shotgun
sequence; 563848676; NZ_AYWU01000030.1
2940; Mesorhizobium sp. L2C089B000 scaffold0011, whole genome shotgun
sequence; 563888034; NZ_AYWV01000011.1
2941; Mesorhizobium sp. L2C084A000 scaffold0007, whole genome shotgun
sequence; 563938926; NZ_AYWX01000007.1
2942; Mesorhizobium sp. L2C067A000 scaffold0014, whole genome shotgun
sequence; 563977521; NZ_AYWY01000014.1
2943; Mesorhizobium sp. L2C066B000 scaffold0012, whole genome shotgun
sequence; 563993080; NZ_AYWZ01000012.1
2944; Mesorhizobium sp. L103C119B0 scaffold0005, whole genome shotgun
sequence; 564005047; NZ_AYXE01000005.1
2945; Mesorhizobium sp. L103C105A0 scaffold0004, whole genome shotgun
sequence; 564008267; NZ_AYXF01000004.1
2946; Xanthomonas hortorum pv. carotae str. M081 chromosome, whole
genome shotgun sequence; 565808720; NZ_CM002307.1
2947; Clostridium pasteurianum NRRL B-598, complete genome; 930593557;
NZ_CP011966.1
2948; Paenibacillus polymyxa CR1, complete genome; 734699963;
NC_023037.2
2949; Streptococcus suis SC84 complete genome, strain SC84; 253750923;
NC_012924.1
2950; Streptococcus suis 10581 Contig00069, whole genome shotgun
sequence; 636868927; NZ_ALKQ01000069.1
2951; Burkholderia pseudomallei HBPUB10134a BP_10134a_103, whole
genome shotgun sequence; 638832186; NZ_AVAL01000102.1
2952; Mycobacterium sp. UM_WGJ Contig_32, whole genome shotgun
sequence; 638971293; NZ_AUWR01000032.1
2953; Mycobacterium iranicum UM_TJL Contig_42, whole genome shotgun
sequence; 638987534; NZ_AUWT01000042.1
2954; Mesorhizobium ciceri CMG6 MescicDRAFT_scaffold_1.2_C, whole
genome shotgun sequence; 639162053; NZ_AWZS01000002.1
2955; Bradyrhizobium sp. ARR65 BraARR65DRAFT_scaffold_9.10_C.
whole genome shotgun sequence; 639168743; NZ_AWZU01000010.1
2956; Paenibacillus sp. MAEPY2 contig7, whole genome shotgun sequence;
639451286; NZ_AWUK01000007.1
2957; Verrucomicrobia bacterium LP2A G346DRAFT_scf7180000000012_quiver.2_C,
whole genome shotgun sequence; 640169055; NZ_JAFS01000002.1
2958; Verrucomicrobia bacterium LP2A G346DRAFT_scf7180000000012_quiver.2_C,
whole genome shotgun sequence; 640169055; NZ_JAFS01000002.1
2959; Robbsia andropogonis Ba3549 160, whole genome shotgun sequence;
640451877; NZ_AYSW01000160.1
2960; Bacillus mannanilyticus JCM 10596, whole genome shotgun sequence;
640600411; NZ_BAMO01000071.1
2961; Bacillus sp. H1a Contig1, whole genome shotgun sequence; 640724079;
NZ_AYMH01000001.1
2962; Enterococcus faecalis ATCC 4200 supercont1.2, whole genome shotgun
sequence; 239948580; NZ_GG670372.1
2963; Enterococcus faecalis EnGen0363 strain RMC5 acAqY-supercont1.4,
whole genome shotgun sequence; 502232520; NZ_KB944632.1
2964; Enterococcus faecalis LA3B-2 Scaffold22, whole genome shotgun
sequence; 522837181; NZ_KE352807.1
2965; Bifidobacterium breve NCFB 2258, complete genome; 749295448;
NZ_CP006714.1
2966; Sphingomonas sanxanigenens NX02, complete genome; 749321911;
NZ_CP006644.1
2967; Nocardia nova SH22a, complete genome; 753809381; NZ_CP006850.1
2968; Kutzneria albida DSM 43870, complete genome; 754862786;
NZ_CP007155.1
2969; Paenibacillus polymyxa SQR-21, complete genome; 749205063;
NZ_CP006872.1
2970; Burkholderia thailandensis E264 chromosome I, complete sequence;
83718394; NC_007651.1
2971; Burkholderia thailandensis H0587 chromosome 1, complete sequence;
759581710; NZ_CP004089.1
2972; Sphingobium barthaii strain KK22, whole genome shotgun sequence;
646523831; NZ_BATN01000047.1
2973; Sphingobium barthaii strain KK22, whole genome shotgun sequence;
646529442; NZ_BATN01000092.1
2974; Paenibacillus polymyxa 1-43 S143_contig00221, whole genome
shotgun sequence; 647225094; NZ_ASRZ01000173.1
2975; Paenibacillus sp. 1-49 S149_contig00281, whole genome shotgun
sequence; 647230448; NZ_ASRY01000102.1
2976; Paenibacillus graminis RSA19 S2_contig00597, whole genome shotgun
sequence; 647256651; NZ_ASSG01000304.1
2977; Paenibacillus sp. 1-18 S118_contig00103, whole genome shotgun
sequence; 647269417; NZ_ASSB01000031.1
2978; Paenibacillus polymyxa TD94 STD94_contig00759, whole genome
shotgun sequence; 647274605; NZ_ASSA01000134.1
2979; Bacillus flexus T6186-2 contig_106, whole genome shotgun sequence;
647636934; NZ_JANV01000106.1
2980; Brevundimonas naejangsanensis strain B1 contig000018, whole genome
shotgun sequence; 647728918; NZ_JHOF01000018.1
2981; Burkholderia thailandensis E555 BTHE555_314, whole genome
shotgun sequence; 485035557; NZ_AECN01000315.1
2982; Burkholderia oklahomensis C6786 chromosome I, complete sequence;
780352952; NZ_CP009555.1
2983; Bacillus endophyticus 2102 contig21, whole genome shotgun sequence;
485049179; NZ_ALIM01000014.1
2984; Methylococcus capsulatus str. Texas = ATCC 19069 strain Texas
contig0129, whole genome shotgun sequence; 483090991;
NZ_AMCE01000064.1
2985; Sphingomonas-like bacterium B12, whole genome shotgun sequence;
484115568; NZ_BACX01000797.1
2986; Nocardiopsis halotolerans DSM 44410 contig_372, whole genome
shotgun sequence; 484016556; NZ_ANAX01000372.1
2987; Nonomuraea coxensis DSM 45129 A3G7DRAFT_scaffold_4.5, whole
genome shotgun sequence; 483454700; NZ_KB903974.1
2988; Streptomyces sp. CcalMP-8W B053DRAFT_scaffold_0.1, whole
genome shotgun sequence; 483961722; NZ_KB890915.1
2989; Spirosoma spitsbergense DSM 19989 B157DRAFT_scaffold_76.77,
whole genome shotgun sequence; 483994857; NZ_KB893599.1
2990; Butyrivibrio sp. XBB1001 G631DRAFT_scaffold00005.5_C, whole
genome shotgun sequence; 651376721; NZ_AUKA01000006.1
2991; Butyrivibrio sp. XPD2002 G587DRAFT_scaffold00011.11, whole
genome shotgun sequence; 651381584; NZ_KE384117.1
2992; Butyrivibrio sp. NC3005 G634DRAFT_scaffold00001.1, whole
genome shotgun sequence; 651394394; NZ_KE384206.1
2993; Butyrivibrio sp. MC2021 T359DRAFT_scaffold00010.10_C, whole
genome shotgun sequence; 651407979; NZ_JHXX01000011.1
2994; Paenarthrobacter nicotinovorans 231Sha2.1M6
I960DRAFT_scaffold00004.4_C, whole genome shotgun sequence;
651445346; NZ_AZVC01000006.1
2995; Bacillus sp. J37 BacJ37DRAFT_scaffold_0.1_C, whole genome
shotgun sequence; 651516582; NZ_JAEK01000001.1
2996; Bacillus sp. J37 BacJ37DRAFT_scaffold_0.1_C, whole genome
shotgun sequence; 651516582; NZ_JAEK01000001.1
2997; Bacillus sp. UNC437CL72CviS29 M014DRAFT_scaffold00009.9_C,
whole genome shotgun sequence; 651596980; NZ_AXVB01000011.1
2998; Butyrivibrio sp. FC2001 G601DRAFT_scaffold00001.1, whole genome
shotgun sequence; 651921804; NZ_KE384132.1
2999; Bacillus bogoriensis ATCC BAA-922
T323DRAFT_scaffold00008.8_C, whole genome shotgun sequence;
651937013; NZ_JHYI01000013.1
3000; Bacillus bogoriensis ATCC BAA-922
T323DRAFT_scaffold00008.8_C, whole genome shotgun sequence;
651937013; NZ_JHYI01000013.1
3001; Bacillus kribbensis DSM 17871 H539DRAFT_scaffold00003.3, whole
genome shotgun sequence; 651983111; NZ_KE387239.1
3002; Fischerella sp. PCC 9431 Fis9431DRAFT_Scaffold1.2, whole genome
shotgun sequence; 652326780; NZ_KE650771.1
3003; Fischerella sp. PCC 9605 FIS9605DRAFT_scaffold2.2, whole genome
shotgun sequence; 652337551; NZ_KI912149.1
3004; Clostridium akagii DSM 12554 BR66DRAFT_scaffold00010.10_C,
whole genome shotgun sequence; 652488076; NZ_JMLK01000014.1
3005; Clostridium beijerinckii HUN142 T483DRAFT_scaffold00004.4, whole
genome shotgun sequence; 652494892; NZ_KK211337.1
3006; Glomeribacter sp. 1016415 H174DRAFT_scaffold00001.1, whole
genome shotgun sequence; 652527059; NZ_KE384226.1
3007; Glomeribacter sp. 1016415 H174DRAFT_scaffold00001.1, whole
genome shotgun sequence; 652527059; NZ_KE384226.1
3008; Mesorhizobium sp. URHA0056 H959DRAFT_scaffold00004.4_C,
whole genome shotgun sequence; 652670206; NZ_AUEL01000005.1
3009; Mesorhizobium loti R88b Meslo2DRAFT_Scaffold1.1, whole genome
shotgun sequence; 652688269; NZ_KI912159.1
3010; Mesorhizobium ciceri WSM4083 MESCI2DRAFT_scaffold_0.1,
whole genome shotgun sequence; 652698054; NZ_KI912610.1
3011; Mesorhizobium sp. URHC0008 N549DRAFT_scaffold00001.1_C,
whole genome shotgun sequence; 652699616; NZ_JIAP01000001.1
3012; Mesorhizobium sp. URHB0007 N550DRAFT_scaffold00001.1_C,
whole genome shotgun sequence; 652714310; NZ_JIAO01000011.1
3013; Mesorhizobium erdmanii USDA 3471 A3AUDRAFT_scaffold_7.8_C,
whole genome shotgun sequence; 652719874; NZ_AXAE01000013.1
3014; Mesorhizobium loti CJ3sym A3A9DRAFT_scaffold_25.26_C, whole
genome shotgun sequence; 652734503; NZ_AXAL01000027.1
3015; Cohnella thermotolerans DSM 17683
G485DRAFT_scaffold00041.41_C, whole genome shotgun sequence;
652787974; NZ_AUCP01000055.1
3016; Cohnella thermotolerans DSM 17683
G485DRAFT_scaffold00041.41_C, whole genome shotgun sequence;
652787974; NZ_AUCP01000055.1
3017; Cohnella thermotolerans DSM 17683 G485DRAFT_scaffold00003.3,
whole genome shotgun sequence; 652794305; NZ_KE386956.1
3018; Lachnospiraceae bacterium NK4A144 G619DRAFT_scaffold00002.2_C,
whole genome shotgun sequence; 652826657; NZ_AUJT01000002.1
3019; Mesorhizobium sp. WSM3626 Mesw3626DRAFT_scaffold_6.7_C,
whole genome shotgun sequence; 652879634; NZ_AZUY01000007.1
3020; Mesorhizobium sp. WSM1293 MesloDRAFT_scaffold_4.5, whole
genome shotgun sequence; 652910347; NZ_KI911320.1
3021; Mesorhizobium sp. WSM3224 YU3DRAFT_scaffold_3.4_C, whole
genome shotgun sequence; 652912253; NZ_ATYO01000004.1
3022; Butyrivibrio fibrisolvens MD2001 G635DRAFT_scaffold00033.33_C,
whole genome shotgun sequence; 652963937; NZ_AUKD01000034.1
3023; Legionella pneumophila subsp. pneumophila strain ATCC 33155
contig032, whole genome shotgun sequence; 652971687;
NZ_JFIN01000032.1
3024; Legionella pneumophila subsp. pneumophila strain ATCC 33154
Scaffold2, whole genome shotgun sequence; 653016013; NZ_KK074241.1
3025; Legionella pneumophila subsp. pneumophila strain ATCC 33823
Scaffold7, whole genome shotgun sequence; 653016661; NZ_KK074199.1
3026; Bacillus sp. URHB0009 H980DRAFT_scaffold00016.16_C, whole
genome shotgun sequence; 653070042; NZ_AUER01000022.1
3027; Lachnospira multipara ATCC 19207 G600DRAFT_scaffold00009.9_C,
whole genome shotgun sequence; 653218978; NZ_AUJG01000009.1
3028; Lachnospira multipara MC2003 T520DRAFT_scaffold00007.7_C,
whole genome shotgun sequence; 653225243; NZ_JHWY01000011.1
3029; Rhodanobacter sp. OR87 RhoOR87DRAFT_scaffold_24.25_C, whole
genome shotgun sequence; 653308965; NZ_AXBJ01000026.1
3030; Rhodanobacter sp. OR92 RhoOR92DRAFT_scaffold_6.7_C, whole
genome shotgun sequence; 653321547; NZ_ATYF01000013.1
3031; Rhodanobacter sp. OR444
RHOOR444DRAFT_NODE_5_len_27336_cov_289_843719.5_C, whole
genome shotgun sequence; 653325317; NZ_ATYD01000005.1
3032; Rhodanobacter sp. OR444
RHOOR444DRAFT_NODE_39_len_52063_cov_320_872864.39, whole
genome shotgun sequence; 653330442; NZ_KE386531.1
3033; Bradyrhizobium sp. WSM1743 YU9DRAFT_scaffold_1.2_C, whole
genome shotgun sequence; 653526890; NZ_AXAZ01000002.1
3034; Bradyrhizobium sp. Aila-2 K288DRAFT_scaffold00086.86_C, whole
genome shotgun sequence; 653556699; NZ_AUEZ01000087.1
3035; Clostridium butyricum AGR2140 G607DRAFT_scaffold00008.8_C,
whole genome shotgun sequence; 653632769; NZ_AUJN01000009.1
3036; Mastigocoleus testanun BC008 Contig-2, whole genome shotgun
sequence; 959926096; NZ_LMTZ01000085.1
3037; [Eubacterium] cellulosolvens LD2006 T358DRAFT_scaffold00002.2_C,
whole genome shotgun sequence; 654392970; NZ_JHXY01000005.1
3038; Desulfatiglans anilini DSM 4660 H567DRAFT_scaffold00005.5_C,
whole genome shotgun sequence; 654868823; NZ_AULM01000005.1
3039; Legionella pneumophila subsp. fraseri strain ATCC 35251 contig031,
whole genome shotgun sequence; 654928151; NZ_JFIG01000031.1
3040; Bacillus sp. FJAT-14578 Scaffold2, whole genome shotgun sequence;
654948246; NZ_KI632505.1
3041; Bacillus sp. J13 PaeJ13DRAFT_scaffold_4.5_C, whole genome shotgun
sequence; 654954291; NZ_JAEO01000006.1
3042; Bacillus sp. 278922_107 H622DRAFT_scaffold00001.1, whole genome
shotgun sequence; 654964612; NZ_KI911354.1
3043; Streptomyces sp. GXT6 genomic scaffold Scaffold4, whole genome
shotgun sequence; 654975403; NZ_KI601366.1
3044; Ruminococcus flavefaciens ATCC 19208 L870DRAFT_scaffold00001.1,
whole genome shotgun sequence; 655069822; NZ_KI912489.1
3045; Paenibacillus sp. UNCCL52 BR01DRAFT_scaffold00001.1, whole
genome shotgun sequence; 655095448; NZ_KK366023.1
3046; Paenibacillus sp. UNC451MF BP97DRAFT_scaffold00018.18_C,
whole genome shotgun sequence; 655103160; NZ_JMLS01000021.1
3047; Paenibacillus pinihumi DSM 23905 = JCM 16419 strain DSM 23905
H583DRAFT_scaffold00005.5, whole genome shotgun sequence; 655115689;
NZ_KE383867.1
3048; Desulfobulbus japonicus DSM 18378 G493DRAFT_scaffold00011.11_C,
whole genome shotgun sequence; 655133038; NZ_AUCV01000014.1
3049; Desulfobulbus mediterraneus DSM 13871 G494DRAFT_scaffold00028.28_C,
whole genome shotgun sequence; 655138083; NZ_AUCW01000035.1
3050; Paenibacillus harenae DSM 16969 H581DRAFT_scaffold00002.2,
whole genome shotgun sequence; 655165706; NZ_KE383843.1
3051; Shimazuella kribbensis DSM 45090 A3GQDRAFT_scaffold_0.1_C,
whole genome shotgun sequence; 655370026; NZ_ATZF01000001.1
3052; Shimazuella kribbensis DSM 45090 A3GQDRAFT_scaffold_5.6_C,
whole genome shotgun sequence; 655371438; NZ_ATZF01000006.1
3053; Streptomyces flavidovirens DSM 40150
G412DRAFT_scaffold00007.7_C, whole genome shotgun sequence;
655414006; NZ_AUBE01000007.1
3054; Streptomyces flavidovirens DSM 40150
G412DRAFT_scaffold00009.9, whole genome shotgun sequence; 655416831;
NZ_KE386846.1
3055; Terasakiella pusilia DSM 6293 Q397DRAFT_scaffold00039.39_C,
whole genome shotgun sequence; 655499373; NZ_JHYO01000039.1
3056; Pseudoxanthomonas suwonensis J43 Psesu2DRAFT_scaffold_44.45_C,
whole genome shotgun sequence; 655566937; NZ_JAES01000046.1
3057; Pseudonocardia acaciae DSM 45401 N912DRAFT_scaffold00002.2_C,
whole genome shotgun sequence; 655569633; NZ_JIAI01000002.1
3058; Azospirillum halopraeferens DSM 3675
G472DRAFT_scaffold00039.39_C, whole genome shotgun sequence;
655967838; NZ_AUCF01000044.1
3059; Clostridium scatologenes strain ATCC 25775, complete genome;
802929558; NZ_CP009933.1
3060; Paenibacillus harenae DSM 16969 H581DRAFT_scaffold00004.4,
whole genome shotgun sequence; 656245934; NZ_KE383845.1
3061; Paenibacillus harenae DSM 16969 H581DRAFT_scaffold00004.4,
whole genome shotgun sequence; 656245934; NZ_KE383845.1
3062; Paenibacillus alginolyticus DSM 5050 = NBRC 15375 strain DSM 5050
G519DRAFT_scaffold00043.43_C, whole genome shotgun sequence;
656249802; NZ_AUGY01000047.1
3063; Bacillus indicus strain DSM 16189 Contig01, whole genome shotgun
sequence; 737222016; NZ_JNVC02000001.1
3064; Acaryochloris sp. CCMEE 5410 contig00232, whole genome shotgun
sequence; 359367134; NZ_AFEJ01000154.1
3065; Bacillus sp. RP1137 contig_18, whole genome shotgun sequence;
657210762; NZ_AXZS01000018.1
3066; Streptomyces leeuwenhoekii strain C34(2013) c34_sequence_0501,
whole genome shotgun sequence; 657301257; NZ_AZSD01000480.1
3067; Brevundimonas bacteroides DSM 4726
Q333DRAFT_scaffold00004.4_C, whole genome shotgun sequence;
657605746; NZ_JNIX01000010.1
3068; Bacillus thuringiensis LM1212 scaffold_08, whole genome shotgun
sequence; 657629081; NZ_AYPV01000024.1
3069; Klebsiella pneumoniae 4541-2 4541_2_67, whole genome shotgun
sequence; 657698352; NZ_JDWO01000067.1
3070; Lachnoclostridium phytofermentans KNHs212
BO10DRAFT_scf7180000000004_quiver.1_C, whole genome shotgun
sequence; 657706549; NZ_JNLM01000001.1
3071; Paenibacillus polymyxa strain WLY78 S6_contig00095, whole genome
shotgun sequence; 657719467; NZ_ALJV01000094.1
3072; Bacillus indicus strain DSM 16189 Contig01, whole genome shotgun
sequence; 737222016; NZ_JNVC02000001.1
3073; [Scytonema hofmanni] UTEX 2349 Tol9009DRAFT_TPD.8, whole
genome shotgun sequence; 657935980; NZ_KK073768.1
3074; Caulobacter sp. UNC358MFTsu5.1 BR39DRAFT_scaffold00002.2_C,
whole genome shotgun sequence; 659864921; NZ_JONW01000006.1
3075; Sphingomonas sp. YL-JM2C contig056, whole genome shotgun
sequence; 661300723; NZ_ASIM01000056.1
3076; Streptomyces monomycini strain NRRL B-24309
P063_Doro1_scaffold135, whole genome shotgun sequence; 662059070;
NZ_KL571162.1
3077; Streptomyces flavotricini strain NRRL B-5419 contig237.1, whole
genome shotgun sequence; 662063073; NZ_JNXV01000303.1
3078; Streptomyces peruviensis strain NRRL ISP-5592
P181_Doro1_scaffold152, whole genome shotgun sequence; 662097244;
NZ_KL575165.1
3079; Sphingomonas sp. DC-6 scaffold87, whole genome shotgun sequence;
662140302; NZ_JMUB01000087.1
3080; Streptomyces sp. NRRL S-455 contig1.1, whole genome shotgun
sequence; 663192162; NZ_JOCT01000001.1
3081; Streptomyces griseoluteus strain NRRL ISP-5360 contig43.1, whole
genome shotgun sequence; 663180071; NZ_JOBE01000043.1
3082; Streptomyces sp. NRRL S-350 contig12.1, whole genome shotgun
sequence; 663199697; NZ_JOHO01000012.1
3083; Streptomyces katrae strain NRRL B-16271 contig37.1, whole genome
shotgun sequence; 663300941; NZ_JNZY01000037.1
3084; Streptomyces sp. NRRL B-3229 contig5.1, whole genome shotgun
sequence; 663316931; NZ_JOGP01000005.1
3085; Streptomyces flavochromogenes strain NRRL B-2684 contig8.1, whole
genome shotgun sequence; 663317502; NZ_JNZO01000008.1
3086; Streptomyces roseoverticillatus strain NRRL B-3500 contig22.1, whole
genome shotgun sequence; 663372343; NZ_JOFL01000022.1
3087; Streptomyces roseoverticillatus strain NRRL B-3500 contig31.1, whole
genome shotgun sequence; 663372947; NZ_JOFL01000031.1
3088; Streptomyces roseoverticillatus strain NRRL B-3500 contig43.1, whole
genome shotgun sequence; 663373497; NZ_JOFL01000043.1
3089; Streptomyces rimosus subsp. rimosus strain NRRL WC-3924 contig19.1,
whole genome shotgun sequence; 663376433; NZ_JOBW01000019.1
3090; Streptomyces rimosus subsp. rimosus strain NRRL WC-3924 contig82.1,
whole genome shotgun sequence; 663379797; NZ_JOBW01000082.1
3091; Streptomyces sp. NRRL B-12105 contig1.1, whole genome shotgun
sequence; 663380895; NZ_JNZW01000001.1
3092; Herbidospora cretacea strain NRRL B-16917 contig7.1, whole genome
shotgun sequence; 663670981; NZ_JODQ01000007.1
3093; Lechevalieria aerocolonigenes strain NRRL B-3298 contig27.1, whole
genome shotgun sequence; 663693444; NZ_JOFJ01000027.1
3094; Microbispora rosea subsp. nonnitritogenes strain NRRL B-2631 contig12.1,
whole genome shotgun sequence; 663732121; NZ_JNZQ01000012.1
3095; Sphingobium sp. DC-2 ODE_45, whole genome shotgun sequence;
663818579; NZ_JNAC01000042.1
3096; Streptomyces aureocirculatus strain NRRL ISP-5386 contig49.1, whole
genome shotgun sequence; 664026629; NZ_JOAP01000049.1
3097; Streptomyces rimosus subsp. rimosus strain NRRL B-2660 contig14.1,
whole genome shotgun sequence; 664052786; NZ_JOES01000014.1
3098; Streptomyces achromogenes subsp. achromogenes strain NRRL B-2120 contig2.1,
whole genome shotgun sequence; 664063830; NZ_JODT01000002.1
3099; Streptomyces rimosus subsp. rimosus strain NRRL B-2660 contig124.1,
whole genome shotgun sequence; 664066234; NZ_JOES01000124.1
3100; Streptomyces rimosus subsp. rimosus strain NRRL WC-3927 contig5.1,
whole genome shotgun sequence; 664091759; NZ_JOBO01000005.1
3101; Streptomyces rimosus subsp. rimosus strain NRRL WC-3869 P248contig50.1,
whole genome shotgun sequence; 925315417; LGCQ01000244.1
3102; Streptomyces rimosus subsp. rimosus strain NRRL WC-3929 contig5.1,
whole genome shotgun sequence; 664104387; NZ_JOJJ01000005.1
3103; Streptomyces rimosus subsp. rimosus strain NRRL WC-3929 contig46.1,
whole genome shotgun sequence; 664115745; NZ_JOJJ01000046.1
3104; Streptomyces rimosus subsp. rimosus strain NRRL WC-3904 contig10.1,
whole genome shotgun sequence; 664126885; NZ_JOCQ01000010.1
3105; Streptomyces rimosus subsp. rimosus strain NRRL WC-3904 contig106.1,
whole genome shotgun sequence; 664141810; NZ_JOCQ01000106.1
3106; Streptomyces sp. NRRL F-2890 contig2.1, whole genome shotgun
sequence; 664194528; NZ_JOIG01000002.1
3107; Streptomyces griseus subsp. griseus strain NRRL F-5618 contig4.1,
whole genome shotgun sequence; 664233412; NZ_JOGN01000004.1
3108; Streptomyces lavenduligriseus strain NRRL ISP-5487 contig2.1, whole
genome shotgun sequence; 664244706; NZ_JOBD01000002.1
3109; Streptomyces sp. NRRL S-920 contig3.1, whole genome shotgun
sequence; 664245663; NZ_JODF01000003.1
3110; Streptomyces hygroscopicus subsp. hygroscopicus strain NRRL B-1477 contig8.1,
whole genome shotgun sequence; 664299296; NZ_JOIK01000008.1
3111; Streptomyces sp. NRRL F-4474 contig32.1, whole genome shotgun
sequence; 664323078; NZ_JOIB01000032.1
3112; Streptomyces sp. NRRL S-475 contig32.1, whole genome shotgun
sequence; 664325162; NZ_JOJB01000032.1
3113; Streptomyces sp. NRRL F-5053 contig1.1, whole genome shotgun
sequence; 664356765; NZ_JOHT01000001.1
3114; Streptomyces sp. NRRL S-1868 contig54.1, whole genome shotgun
sequence; 664360925; NZ_JOGD01000054.1
3115; Streptomyces sp. NRRL S-646 contig23.1, whole genome shotgun
sequence; 664421883; NZ_JODC01000023.1
3116; Streptomyces sp. NRRL S-455 contig1.1, whole genome shotgun
sequence; 663192162; NZ_JOCT01000001.1
3117; Streptomyces sp. NRRL S-481 P269_Doro1_scaffold20, whole genome
shotgun sequence; 664428976; NZ_KL585179.1
3118; Streptomyces sp. NRRL F-5140 contig927.1, whole genome shotgun
sequence; 664434000; NZ_JOIA01001078.1
3119; Streptomyces sp. NRRL WC-3773 contig2.1, whole genome shotgun
sequence; 664478668; NZ_JOJI01000002.1
3120; Streptomyces sp. NRRL WC-3773 contig5.1, whole genome shotgun
sequence; 664479796; NZ_JOJI01000005.1
3121; Streptomyces sp. NRRL WC-3773 contig11.1, whole genome shotgun
sequence; 664481891; NZ_JOJI01000011.1
3122; Streptomyces sp. NRRL WC-3773 contig11.1, whole genome shotgun
sequence; 664481891; NZ_JOJI01000011.1
3123; Streptomyces puniceus strain NRRL ISP-5083 contig3.1, whole genome
shotgun sequence; 663149970; NZ_JOBQ01000003.1
3124; Streptomyces ochraceiscleroticus strain NRRL ISP-5594 contig9.1,
whole genome shotgun sequence; 664540649; NZ_JOAX01000009.1
3125; Streptomyces durhamensis strain NRRL B-3309 contig3.1, whole
genome shotgun sequence; 665586974; NZ_JNXR01000003.1
3126; Streptomyces durhamensis strain NRRL B-3309 contig23.1, whole
genome shotgun sequence; 665604093; NZ_JNXR01000023.1
3127; Streptomyces rimosus subsp. rimosus strain NRRL WC-3869 P248contig20.1,
whole genome shotgun sequence; 925322461; LGCQ01000113.1
3128; Streptomyces niveus NCIMB 11891 chromosome, whole genome
shotgun sequence; 566146291; NZ_CM002280.1
3129; Paenibacillus polymyxa strain CICC 10580 contig_11, whole genome
shotgun sequence; 670516032; NZ_JNCB01000011.1
3130; Streptomyces megasporus strain NRRL B-16372 contig19.1, whole
genome shotgun sequence; 671525382; NZ_JODL01000019.1
3131; Dyadobacter crusticola DSM 16708 Q369DRAFT_scaffold00002.2,
whole genome shotgun sequence; 671546962; NZ_KL370786.1
3132; Bacillus sp. MB2021 T349DRAFT_scaffold00010.10_C, whole
genome shotgun sequence; 671553628; NZ_JNJJ01000011.1
3133; Lachnospira multipara LB2003 T537DRAFT_scaffold00010.10_C,
whole genome shotgun sequence; 671578517; NZ_JNKW01000011.1
3134; Clostridium drakei strain SL1 contig_20, whole genome shotgun
sequence; 692121046; NZ_JIBU02000020.1
3135; Candidatus Paracaedibacter symbiosus strain PRA9 Scaffold_1, whole
genome shotgun sequence; 692233141; NZ_JQAK01000001.1
3136; Stenotrophomonas maltophilia strain 53 contig_2, whole genome
shotgun sequence; 692316574; NZ_JRJA01000002.1
3137; Rhodococcus fascians LMG 3625 contig38, whole genome shotgun
sequence; 694033726; NZ_JMEM01000016.1
3138; Rhodococcus fascians 04-516 contig54, whole genome shotgun
sequence; 694058371; NZ_JMFD01000020.1
3139; Klebsiella michiganensis strain R8A contig_44, whole genome shotgun
sequence; 695806661; NZ_JNCH01000044.1
3140; Streptomyces globisporus C-1027 Scaffold24_1, whole genome shotgun
sequence; 410651191; NZ_AJUO01000171.1
3141; Streptomyces sp. NRRL B-1381 contig33.1, whole genome shotgun
sequence; 663334964; NZ_JOHG01000033.1
3142; Streptomyces sp. SolWspMP-sol2th B083DRAFT_scaffold_17.18_C,
whole genome shotgun sequence; 654969845; NZ_ARPF01000020.1
3143; Streptomyces alboviridis strain NRRL B-1579 contig18.1, whole
genome shotgun sequence; 695845602; NZ_JNWU01000018.1
3144; Streptomyces sp. NRRL F-5681 contig10.1, whole genome shotgun
sequence; 663292631; NZ_JOHA01000010.1
3145; Streptomyces globisporus subsp. globisporus strain NRRL B-2709 contig24.1,
whole genome shotgun sequence; 664051798; NZ_JNZK01000024.1
3146; Streptomyces griseus subsp. griseus strain NRRL F-5144 contig19.1,
whole genome shotgun sequence; 664184565; NZ_JOGA01000019.1
3147; Streptomyces floridae strain NRRL 2423 contig7.1, whole genome
shotgun sequence; 663343774; NZ_JOAC01000007.1
3148; Streptomyces roseosporus NRRL 11379 supercont4.1, whole genome
shotgun sequence; 588273405; NZ_ABYX02000001.1
3149; Streptomyces cyaneofuscatus strain NRRL B-2570 contig9.1, whole
genome shotgun sequence; 664021017; NZ_JOEM01000009.1
3150; Streptomyces sp. NRRL S-623 contig14.1, whole genome shotgun
sequence; 665522165; NZ_JOJC01000016.1
3151; Streptomyces sp. JS01 contig2, whole genome shotgun sequence;
695871554; NZ_JPWW01000002.1
3152; Streptomyces albus subsp. albus strain NRRL B-2445 contig28.1, whole
genome shotgun sequence; 664095100; NZ_JOED01000028.1
3153; Streptomyces baarnensis strain NRRL B-2842 P144_Doro1_scaffold26,
whole genome shotgun sequence; 662135579; NZ_KL573564.1
3154; Streptomyces griseus subsp. griseus strain NRRL F-2227 contig41.1,
whole genome shotgun sequence; 664325626; NZ_JOIT01000041.1
3155; Streptomyces sp. W007 contig00293, whole genome shotgun sequence;
365867746; NZ_AGSW01000272.1
3156; Streptomyces mediolani strain NRRL WC-3934 contig31.1, whole
genome shotgun sequence; 664285409; NZ_JOJK01000031.1
3157; Streptomyces sp. NRRL WC-3773 contig36.1, whole genome shotgun
sequence; 664487325; NZ_JOJI01000036.1
3158; Mesorhizobium japonicum R7A MesloDRAFT_Scaffold1.1, whole
genome shotgun sequence; 696358903; NZ_KI632510.1
3159; Stenotrophomonas maltophilia RA8, whole genome shotgun sequence;
493412056; NZ_CALM01000701.1
3160; Streptomyces rimosus subsp. rimosus strain NRRL B-2660 contig59.1,
whole genome shotgun sequence; 664061406; NZ_JOES01000059.1
3161; Streptomyces rimosus subsp. rimosus strain NRRL B-16073 contig7.1,
whole genome shotgun sequence; 696493030; NZ_JNWX01000007.1
3162; Streptomyces rimosus subsp. rimosus strain NRRL B-16073 contig48.1,
whole genome shotgun sequence; 696497741; NZ_JNWX01000048.1
3163; Sphingopyxis sp. MWB1 contig00002, whole genome shotgun
sequence; 696542396; NZ_JQFJ01000002.1
3164; Blautia producta strain ER3 contig_8, whole genome shotgun sequence;
696661199; NZ_JPJF01000008.1
3165; Streptomyces griseus subsp. griseus strain NRRL B-2307 contig15.1,
whole genome shotgun sequence; 702684649; NZ_JNZI01000015.1
3166; Kitasatospora setae KM-6054 DNA, complete genome; 357386972;
NC_016109.1
3167; Streptomyces lydicus strain NRRL ISP-5461 contig41.1, whole genome
shotgun sequence; 702808005; NZ_JNZA01000041.1
3168; Streptomyces iakyrus strain NRRL ISP-5482 contig6.1, whole genome
shotgun sequence; 702914619; NZ_JNXI01000006.1
3169; Kibdelosporangium aridum subsp. largum strain NRRL B-24462 contig4.56,
whole genome shotgun sequence; 703210604; NZ_JNYM01000124.1
3170; Kibdelosporangium aridum subsp. largum strain NRRL B-24462 contig91.4,
whole genome shotgun sequence; 703243970; NZ_JNYM01001429.1
3171; Xanthomonas campestris pv. viticola strain LMG 965, whole genome
shotgun sequence; 704493846; NZ_CBZT010000006.1
3172; Streptomyces galbus strain KCCM 41354 contig00021, whole genome
shotgun sequence; 716912366; NZ_JRHJ01000016.1
3173; Bacillus aryabhattai strain GZ03 contig1_scaffold1, whole genome
shotgun sequence; 723602665; NZ_JPIE01000001.1
3174; Bacillus mycoides FSL H7-687 Contig052, whole genome shotgun
sequence; 727271768; NZ_ASPY01000052.1
3175; Bacillus mycoides strain Flugge 10206 DJ94.contig-100_16, whole
genome shotgun sequence; 727343482; NZ_JMQD01000030.1
3176; Streptomyces anulatus strain NRRL B-2873 contig21.1, whole genome
shotgun sequence; 664049400; NZ_JOEZ01000021.1
3177; Sphingomonas sp. 37zxx contig3_scaffold2, whole genome shotgun
sequence; 728813405; NZ_JRQH01000003.1
3178; Sphingomonas sp. 35-24ZXX contig11_scaffold1, whole genome
shotgun sequence; 728827031; NZ_JROG01000008.1
3179; Sphingomonas sp. Ant H11 contig_149, whole genome shotgun
sequence; 730274767; NZ_JSBN01000149.1
3180; Sphingomonas sp. ERG5 Contig74, whole genome shotgun sequence;
734983081; NZ_JSXI01000073.1
3181; Sphingomonas sp. ERG5 Contig80, whole genome shotgun sequence;
734983422; NZ_JSXI01000079.1
3182; Bacillus sp. 72 T409DRAFT_scf7180000000077_quiver.15_C, whole
genome shotgun sequence; 736160933; NZ_JQMI01000015.1
3183; Bacillus simplex BA2H3 scaffold2, whole genome shotgun sequence;
736214556; NZ_KN360955.1
3184; Dehalobacter sp. UNSWDEIB Contig_139, whole genome shotgun
sequence; 544905305; NZ_AUUR01000139.1
3185; Bacillus manliponensis strain JCM 15802 contig4, whole genome
shotgun sequence; 736629899; NZ_JOTN01000004.1
3186; Hyphomonas chukchiensis strain BH-BN04-4 contig6, whole genome
shotgun sequence; 736739493; NZ_AWFG01000063.1
3187; Bacillus vietnamensis steam HD-02, w hole genome shotgun sequence;
736762362; NZ_CCDN010000009.1
3188; Hyphomonas sp. CY54-11-8 contig4, whole genome shotgun sequence;
736764136; NZ_AWFD01000033.1
3189; Erythrobacter longus strain DSM 6997 contig9, whole genome shotgun
sequence; 736965849; NZ_JMIW01000009.1
3190; Caulobacter henricii strain CF287 EW90DRAFT_scaffold00023.23_C,
whole genome shotgun sequence; 737089868; NZ_JQJN01000025.1
3191; Caulobacter henricii strain YR570 EX13DRAFT_scaffold00022.22_C,
whole genome shotgun sequence; 737103862; NZ_JQJP01000023.1
3192; Calothrix sp. 336/3, complete genome; 821032128; NZ_CP011382.1
3193; Desulfobacter vibrioformis DSM 8776
Q366DRAFT_scaffold00036.35_C, whole genome shotgun sequence;
737257311; NZ_JQKJ01000036.1
3194; Actinokineospora spheciospongiae strain EG49 contig1268_1, whole
genome shotgun sequence; 737301464; NZ_AYXG01000139.1
3195; Brevundimonas sp. EAKA contig5, whole genome shotgun sequence;
737322991; NZ_JMQR01000005.1
3196; Brevundimonas sp. EAKA contig5, whole genome shotgun sequence;
737322991; NZ_JMQR01000005.1
3197; Brevundimonas sp. EAKA contig12, whole genome shotgun sequence;
737323704; NZ_JMQR01000012.1
3198; Bacillus firmus DS1 scaffold33, whole genome shotgun sequence;
737350949; NZ_APVL01000034.1
3199; Bacillus hemicellulosilyticus JCM 9152, whole genome shotgun
sequence; 737360192; NZ_BAUU01000008.1
3200; Edaphobacter aggregans DSM 19364
Q363DRAFT_scaffold00032.32_C, whole genome shotgun sequence;
737370143; NZ_JQKI01000040.1
3201; Bacillus sp. UNC322MFChir4.1 BR72DRAFT_scaffold00004.4, whole
genome shotgun sequence; 737456981; NZ_KN050811.1
3202; Haloglycomyces albus DSM 45210 HalalDRAFT_chromosome1.1_C,
whole genome shotgun sequence; 644043488; NZ_AZUQ01000001.1
3203; Hyphomonas oceanites SCH89 contig20, whole genome shotgun
sequence; 737567115; NZ_ARYL01000020.1
3204; Hyphomonas oceanites SCH89 contig59, whole genome shotgun
sequence; 737569369; NZ_ARYL01000059.1
3205; Halobacillus sp. BBL2006 cont444, whole genome shotgun sequence;
737576092; NZ_JRNX01000441.1
3206; Hyphomonas atlantica strain 22II1-22F38 contig10, whole genome
shotgun sequence; 737577234; NZ_AWFH01000002.1
3207; Hyphomonas atlantica strain 22II1-22F38 contig28, whole genome
shotgun sequence; 737580759; NZ_AWFH01000021.1
3208; Hyphomonas jannaschiana VP2 contig2, whole genome shotgun
sequence; 737608363; NZ_ARYJ01000002.1
3209; Bacillus akibai JCM 9157, whole genome shotgun sequence;
737696658; NZ_BAUV01000025.1
3210; Frankia sp. CcI6 CcI6DRAFT_scaffold_16.17, whole genome shotgun
sequence; 564016690; NZ_AYTZ01000017.1
3211; Frankia sp. Thr ThrDRAFTscaffold_48.49, whole genome shotgun
sequence; 602261491; JENI01000049.1
3212; Frankia sp. Thr ThrDRAFT_scaffold_28.29, whole genome shotgun
sequence; 602262270; JENI01000029.1
3213; Frankia sp. CcI6 CcI6DRAFT_scaffold_16.17, whole genome shotgun
sequence; 564016690; NZ_AYTZ01000017.1
3214; [Leptolyngbya] sp. JSC-1 Osccy1DRAFT_CYJSC1_DRAF_scaffold00069.1,
whole genome shotgun sequence; 738050739; NZ_KL662191.1
3215; Lysobacter daejeonensis GH1-9 contig23, whole genome shotgun
sequence; 738180952; NZ_AVPU01000014.1
3216; Myxosarcina sp. GI1 contig_5, whole genome shotgun sequence;
738529722; NZ_JRFE01000006.1
3217; Novosphingobium resinovorum stain KF1 contig000002, whole
genome shotgun sequence; 738613868; NZ_JFYZ01000002.1
3218; Novosphingobium resinovorum stain KF1 contig000008, whole
genome shotgun sequence; 738615271; NZ_JFYZ01000008.1
3219; Novosphingobium resinovorum stain KF1 contig000015, whole
genome shotgun sequence; 738617000; NZ_JFYZ01000015.1
3220; Paenibacillus sp. FSL H7-689 Contig015, whole genome shotgun
sequence; 738716739; NZ_ASPU01000015.1
3221; Paenibacillus wynnii strain DSM 18334 unitig_2, whole genome
shotgun sequence; 738760618; NZ_JQCR01000002.1
3222; Pandoraea sp. SD6-2 scaffold29, whole genome shotgun sequence;
505733815; NZ_KB944444.1
3223; Paenibacillus sp. FSL R7-269 Contig022, whole genome shotgun
sequence; 738803633; NZ_ASPS01000022.1
3224; Paenibacillus taiwanensis DSM 18679 H509DRAFT_scaffold00010.10_C,
whole genome shotgun sequence; 655095554; NZ_AULE01000001.1
3225; Paenibacillus sp. FSL R7-277 Contig088, whole genome shotgun
sequence; 738841140; NZ_ASPX01000088.1
3226; Prevotella oryzae DSM 17970 XylorDRAFT_XOA.1, whole genome
shotgun sequence; 738999090; NZ_KK073873.1
3227; Promicromonospora kroppenstedtii DSM 19349 ProkrDRAFT_PKA.71,
whole genome shotgun sequence; 739097522; NZ_KI911740.1
3228; Pseudonocardia acaciae DSM 45401 N912DRAFT_scaffold00002.2_C,
whole genome shotgun sequence; 655569633; NZ_JIAI01000002.1
3229; Rhodanobacter sp. 115 contig437, whole genome shotgun sequence;
389759651; NZ_AJXS01000437.1
3230; Rhodococcus fascians A21d2 contig10, whole genome shotgun
sequence; 739287390; NZ_JMFA01000010.1
3231; Ruminococcus albus 8 contig00035, whole genome shotgun sequence;
325680876; NZ_ADKM02000123.1
3232; Rubellimicrobium mesophilum DSM 19309 scaffold23, whole genome
shotgun sequence; 739419616; NZ_KK088564.1
3233; Rothia aeria F0184 Scaffold136, whole genome shotgun sequence;
553804563; NZ_KI518028.1
3234; Amycolatopsis orientalis DSM 40040 = KCTC 9412 contig_32, whole
genome shotgun sequence; 499136900; NZ_ASJB01000015.1
3235; Amycolatopsis sp. MJM2582 contig00007, whole genome shotgun
sequence; 739487309; NZ_JPLW01000007.1
3236; Sphingobium chlorophenolicum strain NBRC 16172 contig000025,
whole genome shotgun sequence; 739594477; NZ_JFHR01000025.1
3237; Sphingobium chlorophenolicum strain NBRC 16172 contig000062,
whole genome shotgun sequence; 739598481; NZ_JFHR01000062.1
3238; Sphingobium herbicidovorans NBRC 16415 contig000028, whole
genome shotgun sequence; 739610197; NZ_JFZA02000028.1
3239; Sphingobium sp. ba1 seq0028, whole genome shotgun sequence;
739622900; NZ_JPPQ01000069.1
3240; Sphingobium sp. ba1 seq0028, whole genome shotgun sequence;
739622900; NZ_JPPQ01000069.1
3241; Sphingomonas paucimobilis strain EPA505 contig000016, whole
genome shotgun sequence; 739629085; NZ_JFYY01000016.1
3242; Sphingobium japonicum BiD32, whole genome shotgun sequence;
494022722; NZ_CAVK010000217.1
3243; Sphingobium yanoikuyae strain B1 scaffold1, whole genome shotgun
sequence; 739650776; NZ_KL662193.1
3244; Sphingobium yanoikuyae strain B1 scaffold28, whole genome shotgun
sequence; 739656825; NZ_KL662220.1
3245; Sphingopyxis sp. LC81 contig24, whole genome shotgun sequence;
739659070; NZ_JNFD01000017.1
3246; Sphingobium yanoikuyae strain B1 contig000019, whole genome
shotgun sequence; 739665456; NZ_JGVR01000019.1
3247; Sphingomonas wittichii strain YR128 EX04DRAFT_scaffold00050.50_C,
whole genome shotgun sequence; 739674258; NZ_JQMC01000050.1
3248; Sphingopyxis fribergensis strain Kp5.2, complete genome; 749188513;
NZ_CP009122.1
3249; Sphingopyxis sp. LC363 contig30, whole genome shotgun sequence;
739701660; NZ_JNFC01000024.1
3250; Sphingopyxis sp. LC363 contig36, whole genome shotgun sequence;
739702045; NZ_JNFC01000030.1
3251; Sphingopyxis sp. LC363 contig5, whole genome shotgun sequence;
739702995; NZ_JNFC01000045.1
3252; Spirillospora albida strain NRRL B-3350 contig1.1, whole genome
shotgun sequence; 663122276; NZ_JOFJ01000001.1
3253; Sphingomonas sp. UNC305MFCol5.2 BR78DRAFT_scaffold00001.1_C,
whole genome shotgun sequence; 659889283; NZ_JOOE01000001.1
3254; Streptococcus salivarius strain NU10 contig_11, whole genome shotgun
sequence; 739748927; NZ_JJMT01000011.1
3255; Streptomyces katrae strain NRRL B-16271 contig33.1, whole genome
shotgun sequence; 663300513; NZ_JNZY01000033.1
3256; Streptomyces avermitilis MA-4680 = NBRC 14893, complete genome;
162960844; NC_003155.4
3257; Streptomyces avermitilis MA-4680 = NBRC 14893, complete genome;
162960844; NC_003155.4
3258; Streptomyces aurantiacus JA 4570 Seq17, whole genome shotgun
sequence; 514916021; NZ_AOPZ01000017.1
3259; Streptomyces griseus subsp. griseus strain NRRL WC-3645 contig39.1,
whole genome shotgun sequence; 739830131; NZ_JOJE01000039.1
3260; Streptomyces griseus subsp. griseus strain NRRL WC-3645 contig40.1,
whole genome shotgun sequence; 739830264; NZ_JOJE01000040.1
3261; Streptomyces aureocirculatus strain NRRL ISP-5386 contig11.1, whole
genome shotgun sequence; 664013282; NZ_JOAP01000011.1
3262; Streptomyces scabiei strain NCPPB 4086 scf_65433_365.1, whole
genome shotgun sequence; 739854483; NZ_KL997447.1
3263; Streptomyces sp. ATexAB-D23 B082DRAFT_scaffold_0.1, whole
genome shotgun sequence; 483975550; NZ_KB892001.1
3264; Streptomyces lavendulae strain Fujisawa #8006 contig417.1, whole
genome shotgun sequence; 662043624; NZ_JNXL01000469.1
3265; Streptomyces sp. DpondAA-B6 K379DRAFT_scaffold00015.15_C,
whole genome shotgun sequence; 654993549; NZ_AZVE01000016.1
3266; Streptomyces sclerotialus strain NRRL B-2317 contig7.1, whole genome
shotgun sequence; 664034500; NZ_JODX01000007.1
3267; Streptomyces olindensis strain DAUFPE 5622 103, whole genome
shotgun sequence; 739918964; NZ_JJOH01000097.1
3268; Streptomyces pristinaespiralis ATCC 25486 chromosome, whole
genome shotgun sequence; 297189896; NZ_CM000950.1
3269; Streptomyces sp. CNH099 B121DRAFT_scaffold_16.17_C, whole
genome shotgun sequence; 654239557; NZ_AZWL01000018.1
3270; Streptomyces sp. MspMP-M5 B073DRAFT_scaffold_27.28, whole
genome shotgun sequence; 483974021; NZ_KB891893.1
3271; Streptomyces sp. NRRL S-1813 contig13.1, whole genome shotgun
sequence; 664466568; NZ_JOHB01000013.1
3272; Streptomyces sp. NRRL S-87 contig69.1, whole genome shotgun
sequence; 663169513; NZ_JO
3273; Streptomyces sp. PRh5 contig001, whole genome shotgun sequence;
740097110; NZ_JABQ01000001.1
3274; Thioclava dalianensis strain DLFJ1-1 contig2, whole genome shotgun
sequence; 740220529; NZ_JHEH01000002.1
3275; Tolypothrix bouteillei VB521301 scaffold_1, whole genome shotgun
sequence; 910242069; NZ_JHEG02000048.1
3276; Thioclava indica strain DT23-4 contig29, whole genome shotgun
sequence; 740292158; NZ_AUNB01000028.1
3277; Streptomyces albulus strain NK660, complete genome; 754221033;
NZ_CP007574.1
3278; Paenibacillus sp. FSL H7-0357, complete genome; 749299172;
NZ_CP009241.1
3279; Paenibacillus stellifer strain DSM 14472, complete genome; 753871514;
NZ_CP009286.1
3280; Burkholderia pseudomallei 1258a Contig0089, whole genome shotgun
sequence; 418540998; NZ_AHJB01000089.1
3281; Burkholderia pseudomallei ABCPW 91 scaffold1, whole genome
shotgun sequence; 740941050; NZ_KN323016.1
3282; Burkholderia pseudomallei strain MSHR4018 scaffold2, whole genome
shotgun sequence; 740942724; NZ_KN323080.1
3283; Burkholderia pseudomallei MSHR1357 scaffold1, whole genome
shotgun sequence; 740944663; NZ_KN323054.1
3284; Burkholderia pseudomallei ABCPW 30 scaffold1, whole genome
shotgun sequence; 740947478; NZ_KN323024.1
3285; Burkholderia pseudomallei MSHR465J scaffold1, whole genome
shotgun sequence; 740992312; NZ_KN322994.1
3286; Burkholderia pseudomallei TSV32 Y025.contig-100_19, whole genome
shotgun sequence; 740951623; NZ_JQHT01000093.1
3287; Burkholderia pseudomallei MSHR2990 scaffold2, whole genome
shotgun sequence; 740957131; NZ_KN323051.1
3288; Burkholderia sp. ABCPW 111 X946.contig-100_0, whole genome
shotgun sequence; 740958729; NZ_JPWT01000001.1
3289; Burkholderia pseudomallei MSHR1000 scaffold1, whole genome
shotgun sequence; 740963677; NZ_KN323065.1
3290; Burkholderia pseudomallei strain BDM scaffold1, whole genome
shotgun sequence; 740964046; NZ_KN150935.1
3291; Burkholderia pseudomallei strain BEG scaffold1, whole genome
shotgun sequence; 740978899; NZ_KN150957.1
3292; Burkholderia pseudomallei strain BDZ scaffold40, whole genome
shotgun sequence; 740989169; NZ_KN150904.1
3293; Burkholderia pseudomallei MSHR4377 scaffold1, whole genome
shotgun sequence; 740998359; NZ_KN322996.1
3294; Burkholderia pseudomallei strain BGH scaffold1, whole genome
shotgun sequence; 741001323; NZ_KN150943.1
3295; Burkholderia pseudomallei MSHR7343 X962.contig-100_14, whole
genome shotgun sequence; 741003124; NZ_JQDM01000047.1
3296; Burkholderia pseudomallei strain PFGE_B T6 scaffold1, whole genome
shotgun sequence; 741007242; NZ_KN150983.1
3297; Burkholderia pseudomallei MSHR3965 chromosome 1 sequence;
752520733; NZ_CP009153.1
3298; Burkholderia pseudomallei MSHR5492 X992.contig-100_0, whole
genome shotgun sequence; 741015160; NZ_JQDO01000001.1
3299; Burkholderia oklahomensis strain EO147 chromosome 1, complete
sequence; 752612400; NZ_CP008726.1
3300; Burkholderia oklahomensis strain EO147 chromosome 1, complete
sequence; 752612400; NZ_CP008726.1
3301; Cupriavidus sp. IDO NODE_7, whole genome shotgun sequence;
742878908; NZ_JWMA01000006.1
3302; Cupriavidus sp. IDO NODE_7, whole genome shotgun sequence;
742878908; NZ_JWMA01000006.1
3303; Escherichia coli strain EC2_3 Contig93, whole genome shotgun
sequence; 742921760; NZ_JWKL01000093.1
3304; Brevundimonas nasdae strain TPW30 Contig_11, whole genome
shotgun sequence; 746187486; NZ_JWSY01000011.1
3305; Brevundimonas nasdae strain TPW30 Contig_13, whole genome
shotgun sequence; 746187665; NZ_JWSY01000013.1
3306; Paenibacillus polymyxa strain DSM 365 Contig001, whole genome
shotgun sequence; 746220937; NZ_JMIQ01000001.1
3307; Paenibacillus polymyxa strain CF05 genome; 746228615;
NZ_CP009909.1
3308; Novosphingobium malaysiense strain MUSC 273 Contig11, whole
genome shotgun sequence; 746242072; NZ_JTDI01000011.1
3309; Paenibacillus sp. IHB B 3415 contig_069, whole genome shotgun
sequence; 746258261; NZ_JUEI01000069.1
3310; Novosphingobium subterraneum strain DSM 12447
NJ75_contig000013, whole genome shotgun sequence; 746288194;
NZ_JRVC01000013.1
3311; Pandoraea sputorum strain DSM 21091, complete genome; 749204399;
NZ_CP010431.1
3312; Xanthomonas cannabis pv. cannabis strain NCPPB 3753 contig_67,
whole genome shotgun sequence; 746366822; NZ_JSZF01000067.1
3313; Xanthomonas arboricola pv. pruni MAFF 301420 strain MAFF301420,
whole genome shotgun sequence; 759376814; NZ_BAVC01000017.1
3314; Xanthomonas arboricola pv. celebensis strain NCPPB 1630
scf_49108_10.1, whole genome shotgun sequence; 746486416;
NZ_KL638873.1
3315; Xanthomonas arboricola pv. celebensis strain NCPPB 1832
scf_23466_141.1, whole genome shotgun sequence; 746494072;
NZ_KL638866.1
3316; Xanthomonas cannabis pv. cannabis strain NCPPB 2877 contig_94,
whole genome shotgun sequence; 746532813; NZ_JSZE01000094.1
3317; Sphingopyxis fribergensis strain Kp5.2, complete genome; 749188513;
NZ_CP009122.1
3318; Sphingopyxis fribergensis strain Kp5.2, complete genome; 749188513;
NZ_CP009122.1
3319; Sphingopyxis fribergensis strain Kp5.2, complete genome; 749188513;
NZ_CP009122.1
3320; Xanthomonas phaseoli pv. phaseoli strain NCPPB 557
scf_22337_104.contig 1, whole genome shotgun sequence; 821373081;
NZ_JWTE02000036.1
3321; Corynebacterium minutissimum strain ATCC 23348
Ordered Contig 015, whole genome shotgun sequence; 746717390;
NZ_JSEF01000015.1
3322; Hassallia byssoidea VB512170 scaffold_0, whole genome shotgun
sequence; 748181452; NZ_JTCM01000043.1
3323; Xanthomonas arboricola pv. corylina str. NCCB 100457 Contig50,
whole genome shotgun sequence; 507418017; NZ_APMC02000050.1
3324; Paenibacillus sonchi X19-5 S5_contig01138, whole genome shotgun
sequence; 484099183; NZ_AJTY01001072.1
3325; Pedobacter sp. BAL39 1103467000492, whole genome shotgun
sequence; 149277373; NZ_ABCM01000005.1
3326; Paenibacillus sp. FSL R7-0273, complete genome; 749302091;
NZ_CP009283.1
3327; Amycolatopsis decaplanina DSM 44594 Contig0055, whole genome
shotgun sequence; 458848256; NZ_AOHO01000055.1
3328; Rhodanobacter thiooxydans LCS2 contig057, whole genome shotgun
sequence; 389809081; NZ_AJXW01000057.1
3329; Bacillus sp. REN51N contig_2, whole genome shotgun sequence;
748816024; NZ_JXAB01000002.1
3330; Paenibacillus polymyxa strain Sb3-1, complete genome; 749204146;
NZ_CP010268.1
3331; Citrobacter pasteurii strain CIP 55.13, whole genome shotgun sequence;
749611130; NZ_CDHL01000044.1
3332; Klebsiella pneumoniae CCHB01000016, whole genome shotgun
sequence; 749639368; NZ_CCHB01000016.1
3333; Streptomonospora alba strain YIM 90003 contig_9, whole genome
shotgun sequence; 749673329; NZ_JROO01000009.1
3334; Actinobaculum sp. oral taxon 183 str. F0552 Scaffold1, whole genome
shotgun sequence; 545327174; NZ_KE951406.1
3335; Actinobaculum sp. oral taxon 183 str. F0552 Scaffold15, whole genome
shotgun sequence; 545327527; NZ_KE951412.1
3336; Rubidibacter lacunae KORDI 51-2 KR51_contig00121, whole genome
shotgun sequence; 550281965; NZ_ASSJ01000070.1
3337; Nocardiopsis chromatogenes YIM 90109 contig_93, whole genome
shotgun sequence; 484026206; NZ_ANBH01000093.1
3338; Streptomyces auratus AGR0001 Scaffold1, whole genome shotgun
sequence; 398790069; NZ_JH725387.1
3339; Gorillibacterium massiliense strain G5, whole genome shotgun
sequence; 750677319; NZ_CBQR020000171.1
3340; Mesorhizobium sp. ORS3324, whole genome shotgun sequence;
751265275; NZ_CCMY01000220.1
3341; Mesorhizobium plurifarium, whole genome shotgun sequence;
751280166; NZ_CCNB01000034.1
3342; Mesorhizobium sp. SOD10, whole genome shotgun sequence;
751285871; NZ_CCNA01000001.1
3343; Mesorhizobium plurifarium, whole genome shotgun sequence;
751292755; NZ_CCNE01000004.1
3344; Mesorhizobium plurifarium, whole genome shotgun sequence;
751299847; NZ_CCMZ01000015.1
3345; Tolypothrix campylonemoides VB511288 scaffold_0, whole genome
shotgun sequence; 751565075; NZ_JXCB01000004.1
3346; Jeotgalibacillus campisalis strain SF-57 contig00001, whole genome
shotgun sequence; 751586078; NZ_JXRR01000001.1
3347; Cohnella kolymensis strain VKM B-2846 B2846_22, whole genome
shotgun sequence; 751596254; NZ_JXAL01000022.1
3348; Jeotgalibacillus soli strain P9 contig00009, whole genome shotgun
sequence; 751619763; NZ_JXRP01000009.1
3349; Burkholderia pseudomallei MSHR4000 scaffold1, whole genome
shotgun sequence; 752517538; NZ_KN323041.1
3350; Burkholderia pseudomallei MSHR4303 scaffold1, whole genome
shotgun sequence; 752519380; NZ_KN323039.1
3351; Burkholderia pseudomallei MSHR4300 scaffold1, whole genome
shotgun sequence; 752522535; NZ_KN322998.1
3352; Burkholderia pseudomallei MSHR4032 scaffold1, whole genome
shotgun sequence; 752526735; NZ_KN323008.1
3353; Geobacter uraniireducens Rf4, complete genome; 148262085;
NC_009483.1
3354; Saccharothrix espanaensis DSM 44229 complete genome; 433601838;
NC_019673.1
3355; Mycobacterium sinense strain JDM601, complete genome; 333988640;
NC_015576.1
3356; Sphingomonas wittichii RW1, complete genome; 148552929;
NC_009511.1
3357; Sphingopyxis alaskensis RB2256, complete genome; 103485498;
NC_008048.1
3358; Sphingopyxis alaskensis RB2256, complete genome; 103485498;
NC_008048.1
3359; Synechococcus sp. PCC 6312, complete genome; 427711179;
NC_019680.1
3360; Caulobacter sp. K31, complete genome; 167643973; NC_010338.1
3361; Tistrella mobilis KA081020-065 plasmid pTM1, complete sequence;
442559580; NC_017957.2
3362; Stackebrandtia nassauensis DSM 44728, complete genome; 291297538;
NC_013947.1
3363; Magnetospirillum gryphiswaldense MSR-1 v2, complete genome;
568144401; NC_023065.1
3364; Asticcacaulis excentricus CB 48 chromosome 1, complete sequence;
315497051; NC_014816.1
3365; Emticicia oligotrophica DSM 17448, complete genome; 408671769;
NC_018748.1
3366; Clostridium beijerinckii strain NCIMB 14988 genome; 754484184;
NZ_CP010086.1
3367; Desulfocapsa sulfexigens DSM 10523, complete genome; 451945650;
NC_020304.1
3368; Gallionella capsiferriformans ES-2, complete genome; 302877245;
NC_014394.1
3369; Paenibacillus sp. FSL P4-0081, complete genome; 754777894;
NZ_CP009280.1
3370; Streptomyces sp. NBRC 110027, whole genome shotgun sequence;
754788309; NZ_BBNO01000002.1
3371; Streptomyces sp. NBRC 110027, whole genome shotgun sequence;
754796661; NZ_BBNO01000008.1
3372; Paenibacillus sp. FSL R7-0331, complete genome; 754821094;
NZ_CP009284.1
3373; Paenibacillus camerounensis strain G4, whole genome shotgun
sequence; 754841195; NZ_CCDG010000069.1
3374; Paenibacillus borealis strain DSM 13188, complete genome;
754859657; NZ_CP009285.1
3375; Paenibacillus sp. FSL R5-0912, complete genome; 754884871;
NZ_CP009282.1
3376; Legionella pneumophila serogroup 1 strain TUM 13948, whole genome
shotgun sequence; 754875479; NZ_BAYQ01000013.1
3377; Nocardiopsis sp. TP-A0876 strain NBRC 110039, whole genome
shotgun sequence; 754924215; NZ_BAZE01000001.1
3378; Streptacidiphilus neutrinimicus strain NBRC 100921, whole genome
shotgun sequence; 755016073; NZ_BBPO01000030.1
3379; Sanguibacter keddieii DSM 10542, complete genome; 269793358;
NC_013521.1
3380; Streptacidiphilus jiangxiensis strain NBRC 100920, whole genome
shotgun sequence; 755108320; NZ_BBPN01000056.1
3381; Mesorhizobium sp. ORS3359, whole genome shotgun sequence;
756828038; NZ_CCNC01000143.1
3382; Streptomyces rimosus strain R6-500MV9-R8 contig021, whole genome
shotgun sequence; 757577710; NZ_JMGY01000021.1
3383; Burkholderia pseudomallei Bp22 chromosome I, whole genome shotgun
sequence; 485065055; NZ_CM001156.1
3384; Bacillus mycoides strain BHP DJ93.Contig42, whole genome shotgun
sequence; 757763573; NZ_JMQC01000008.1
3385; Aneurinibacillus migulanus strain NCTC 7096 contig_153, whole
genome shotgun sequence; 759007555; NZ_JYBO01000079.1
3386; Xanthomonas arboricola pv. pruni strain Xap33 contig_176, whole
genome shotgun sequence; 759358038; NZ_JHUQ01000175.1
3387; Sphingobium sp. Ant17 Contig_45, whole genome shotgun sequence;
759429528; NZ_JEMV01000036.1
3388; Sphingobium sp. Ant17 Contig_90, whole genome shotgun sequence;
759431957; NZ_JEMV01000094.1
3389; Bifidobacterium callitrichos DSM 23973 contig4, whole genome
shotgun sequence; 759443001; NZ_JDUV01000004.1
3390; Streptomyces sp. NRRL F-5123 contig24.1, whole genome shotgun
sequence; 671535174; NZ_JOHY01000024.1
3391; Streptomyces vinaceus strain NRRL ISP-5257 contig5.1, whole genome
shotgun sequence; 759527818; NZ_JNYP01000005.1
3392; Burkholderia pseudomallei MSHR1153 chromosome 1, complete
sequence; 759555751; NZ_CP009271.1
3393; Burkholderia thailandensis MSMB43 Scaffold3, whole genome shotgun
sequence; 424903876; NZ_JH692063.1
3394; Pseudomonas sp. HMP271 Pseudomonas_HMP271_contig_7, whole
genome shotgun sequence; 759578528; NZ_JMFZ01000007.1
3395; Stenotrophomonas maltophilia strain B418 Contig_4_, whole genome
shotgun sequence; 759679095; NZ_JSXG01000004.1
3396; Kitasatospora sp. MBT66 scaffold3, whole genome shotgun sequence;
759755931; NZ_JAIY01000003.1
3397; Streptomyces bingchenggensis BCW-1, complete genome; 374982757;
NC_016582.1
3398; Streptomyces glaucescens strain GLA.O, complete genome; 759802587;
NZ_CP009438.1
3399; Streptomyces glaucescens strain GLA.O, complete genome; 759802587;
NZ_CP009438.1
3400; Actinomyces israelii DSM 43320 O145DRAFT_scaffold00014.14_C,
whole genome shotgun sequence; 759875025; NZ_JONS01000016.1
3401; Rubrivivax gelatinosus IL144 DNA, complete genome; 383755859;
NC_017075.1
3402; Clostridium butyricum strain HM-68 Contig83, whole genome shotgun
sequence; 760273878; NZ_JXBT01000001.1
3403; Novosphingobium sp. P6W scaffold3, whole genome shotgun sequence;
763092879; NZ_JXZE01000003.1
3404; Streptomyces fulvissimus DSM 40593, complete genome; 488607535;
NC_021177.1
3405; Novosphingobium sp. P6W scaffold9, whole genome shotgun sequence;
763095630; NZ_JXZE01000009.1
3406; Bifidobacterium reuteri DSM 23975 contig4, whole genome shotgun
sequence; 763216595; NZ_JDUW01000004.1
3407; Sphingomonas hengshuiensis strain WHSC-8, complete genome;
764364074; NZ_CP010836.1
3408; Sphingomonas hengshuiensis strain WHSC-8, complete genome;
764364074; NZ_CP010836.1
3409; Burkholderia pseudomallei strain QCMRI_BP13 Contig_7, whole
genome shotgun sequence; 764427571; NZ_JYBH01000021.1
3410; Streptomyces natalensis ATCC 27448 Scaffold_33, whole genome
shotgun sequence; 764439507; NZ_JRKI01000027.1
3411; Streptomyces griseus strain S4-7 contig113, whole genome shotgun
sequence; 764464761; NZ_JYBE01000113.1
3412; Streptomyces cyaneogriseus subsp. noncyanogenus strain NMWT 1,
complete genome; 764487836; NZ_CP010849.1
3413; Bacillus subtilis subsp. spizizenii RFWG1A4 contig00010, whole
genome shotgun sequence; 764657375; NZ_AJHM01000010.1
3414; Mastigocladus laminosus UU774 scaffold_22, whole genome shotgun
sequence; 764671177; NZ_JXIJ01000139.1
3415; Bacillus subtilis subsp. spizizenii RFWG5B15 contig00010, whole
genome shotgun sequence; 764677272; NZ_AJHO01000010.1
3416; Streptomyces iranensis genome assembly Siranensis, scaffold
SCAF00002; 765016627; NZ_LK022849.1
3417; Risungbinella massiliensis strain GD1, whole genome shotgun sequence;
765315585; NZ_LN812103.1
3418; Risungbinella massiliensis strain GD1, whole genome shotgun sequence;
765315585; NZ_LN812103.1
3419; Paenibacillus terrae strain NRRL B-30644 contig00007, whole genome
shotgun sequence; 765319397; NZ_JTHP01000007.1
3420; Sphingobium sp. YBL2, complete genome; 765344939;
NZ_CP010954.1
3421; Sphingobium sp. YBL2, complete genome; 765344939;
NZ_CP010954.1
3422; Streptococcus suis strain LS5J, whole genome shotgun sequence;
765394696; NZ_CEEZ01000028.1
3423; Bacillus mycoides strain 11kri323 LG56_082, whole genome shotgun
sequence; 765533368; NZ_JYCJ01000082.1
3424; Streptococcus suis strain LS8F, whole genome shotgun sequence;
766589647; NZ_CEHJ01000007.1
3425; Streptococcus suis strain LS8I, whole genome shotgun sequence;
766595491; NZ_CEHM01000004.1
3426; Paenibacillus polymyxa strain NRRL B-30509 contig00003, whole
genome shotgun sequence; 766607514; NZ_JTHO01000003.1
3427; Thalassospira sp. HJ NODE_2, whole genome shotgun sequence;
766668420; NZ_JYII01000010.1
3428; Paenibacillus sp. IHBB 10380, complete genome; 767005659;
NZ_CP010976.1
3429; Frankia sp. CpI1-S FF36_scaffold_9.10, whole genome shotgun
sequence; 768715243; NZ_JYFN01000010.1
3430; Streptococcus suis strain B28P, whole genome shotgun sequence;
769231516; NZ_CDTB01000010.1
3431; Lechevalieria aerocolonigenes strain NRRL B-16140 contig11.3, whole
genome shotgun sequence; 772744565; NZ_JYJG01000059.1
3432; Streptomyces sp. NRRL F-4428 contig40.2, whole genome shotgun
sequence; 772774737; NZ_JYJI01000131.1
3433; Bacterium endosymbiont of Mortierella elongata FMR23-6, whole
genome shotgun sequence; 779889750; NZ_DF850521.1
3434; Bacterium endosymbiont of Mortierella elongata FMR23-6, whole
genome shotgun sequence; 779889750; NZ_DF850521.1
3435; Streptomyces sp. FxanaA7 F611DRAFT_scaffold00041.41_C, whole
genome shotgun sequence; 780340655; NZ_LACL01000054.1
3436; Burkholderia oklahomensis C6786 chromosome I, complete sequence;
780352952; NZ_CP009555.1
3437; Burkholderia pseudomallei MSHR2543 chromosome I, complete
sequence; 782642065; NZ_CP009478.1
3438; Burkholderia thailandensis 34 chromosome 1, complete sequence;
782674607; NZ_CP010017.1
3439; Streptomyces rubellomurinus strain ATCC 31215 contig-63, whole
genome shotgun sequence; 783211546; NZ_JZKH01000064.1
3440; Burkholderia pseudomallei strain MSHR5107 Contig_3, whole genome
shotgun sequence; 785595141; NZ_JZXP01000013.1
3441; Elstera litoralis strain Dia-1 c21, whole genome shotgun sequence;
788026242; NZ_LAJY01000021.1
3442; Frankia sp. DC12 FraDC12DRAFT_scaffold1.1, whole genome
shotgun sequence; 797224947; NZ_KQ031391.1
3443; Sphingomonas sp. SRS2 contig40, whole genome shotgun sequence;
806905234; NZ_LARW01000040.1
3444; Bacillus sp. UMTAT18 contig00001 1, whole genome shotgun
sequence; 806951735; NZ_JSFD01000011.1
3445; Paenibacillus wulumuqiensis strain Y24 Scaffold4, whole genome
shotgun sequence; 808051893; NZ_KQ040793.1
3446; Bacillus endophyticus strain Hbe603, complete genome; 890672806;
NZ_CP011974.1
3447; Paenibacillus algorifonticola strain XJ259 Scaffold20_1, whole genome
shotgun sequence; 808072221; NZ_LAQO01000025.1
3448; Streptomyces sp. MBT28 contig_50, whole genome shotgun sequence;
808090008; NZ_LARV01000050.1
3449; Mycobacterium sp. UM_Kg27 contig000002, whole genome shotgun
sequence; 809025315; NZ_JRMM01000002.1
3450; Mycobacterium sp. UM_Kg1 contig000164, whole genome shotgun
sequence; 809073490; NZ_JRMK01000164.1
3451; Xanthomonas campestris strain 17, complete genome; 810489403;
NZ_CP011256.1
3452; Spirosoma radiotolerans strain DG5A, complete genome; 817524426;
NZ_CP010429.1
3453; Allosalinactinospora lopnorensis strain CA15-2 contig00044, whole
genome shotgun sequence; 815863894; NZ_LAJC01000044.1
3454; Bacillus sp. SA1-12 scf7180000003378, whole genome shotgun
sequence; 817541164; NZ_LATZ01000026.1
3455; Streptomyces xiamenensis strain 318, complete genome; 921170702;
NZ_CP009922.2
3456; Streptomyces xiamenensis strain 318, complete genome; 921170702;
NZ_CP009922.2
3457; Altererythrobacter atlanticus strain 26DY36, complete genome;
927872504; NZ_CP011452.2
3458; Streptomyces lydicus A02, complete genome; 822214995;
NZ_CP007699.1
3459; Streptomyces lydicus A02, complete genome; 822214995;
NZ_CP007699.1
3460; Streptomyces lydicus A02, complete genome; 822214995;
NZ_CP007699.1
3461; Bacillus cereus strain B4147 NODE_5, whole genome shotgun
sequence; 822530609; NZ_LCYN01000004.1
3462; Xanthomonas pisi DSM 18956 Contig_28, whole genome shotgun
sequence; 822535978; NZ_JPLE01000028.1
3463; Erythrobacter luteus strain KA37 contig1, whole genome shotgun
sequence; 822631216; NZ_LBHB01000001.1
3464; Erythrobacter marinus strain KCTC 23554 KCTC23554_C3, whole
genome shotgun sequence; 829088381; NZ_LDCP01000003.1
3465; Streptomyces leeuwenhoekii strain C34(2013) c34_sequence_0041,
whole genome shotgun sequence; 657295264; NZ_AZSD01000040.1
3466; Streptomyces leeuwenhoekii strain C34(2013) c34_sequence_0012,
whole genome shotgun sequence; 657294764; NZ_AZSD01000012.1
3467; Xanthomonas arboricola strain CFBP 7634 Xarjug-CFBP7634-G11,
whole genome shotgun sequence; 825139250; NZ_JZEH01000001.1
3468; Xanthomonas arboricola strain CFBP 7651 Xarjug-CFBP7651-G11,
whole genome shotgun sequence; 825156557; NZ_JZEI01000001.1
3469; Luteimonas sp. FCS-9 scf7180000000225, whole genome shotgun
sequence; 825314716; NZ_LASZ01000002.1
3470; Luteimonas sp. FCS-9 scf7180000000226, whole genome shotgun
sequence; 825314728; NZ_LASZ01000003.1
3471; Streptomyces sp. KE1 Contig11, whole genome shotgun sequence;
825353621; NZ_LAYX01000011.1
3472; Frankia coriariae strain BMG5.1 scaffold41.42, whole genome shotgun
sequence; 827465632; NZ_JWIO01000042.1
3473; Erythrobacter marinus strain KCTC 23554 KCTC23554_C3, whole
genome shotgun sequence; 829088381; NZ_LDCP01000003.1
3474; Alistipes sp. ZOR0009 L990_140, whole genome shotgun sequence;
835319962; NZ_JTLD01000119.1
3475; Streptomyces sp. M10 Scaffold2, whole genome shotgun sequence;
835355240; NZ_KN549147.1
3476; Bacillus aryabhattai strain T61 Scaffold1, whole genome shotgun
sequence; 836596561; NZ_KQ087173.1
3477; Croceicoccus naphthovorans strain PQ-2, complete genome;
836676868; NZ_CP011770.1
3478; Paenibacillus sp. TCA20, whole genome shotgun sequence; 843088522;
NZ_BBIW01000001.1
3479; Bacillus circulans strain RIT379 contig11, whole genome shotgun
sequence; 844809159; NZ_LDPH01000011.1
3480; Ornithinibacillus californiensis strain DSM 16628 contig_22, whole
genome shotgun sequence; 849059098; NZ_LDUE01000022.1
3481; Bacillus pseudalcaliphilus strain DSM 8725 super11, whole genome
shotgun sequence; 849078078; NZ_LFJO01000006.1
3482; Bacillus aryabhattai strain LK25 16, whole genome shotgun sequence;
850356871; NZ_LDWN01000016.1
3483; Methanobacterium sp. SMA-27 DL91DRAFT_unitig_0_quiver.1_C,
whole genome shotgun sequence; 851351157; NZ_JQLY01000001.1
3484; Cellulomonas sp. A375-1 contig_129, whole genome shotgun sequence;
856992287; NZ_LFKW01000127.1
3485; Bacillus cereus strain RIMV BC 126 212, whole genome shotgun
sequence; 872696015; NZ_LABO01000035.1
3486; Streptomyces leeuwenhoekii strain C58 contig69, whole genome
shotgun sequence; 873282617; NZ_LFEH01000068.1
3487; Streptomyces leeuwenhoekii strain C58 contig126, whole genome
shotgun sequence; 873282818; NZ_LFEH01000123.1
3488; Sphingomonas sp. MEA3-1 contig00021, whole genome shotgun
sequence; 873296042; NZ_LECE01000021.1
3489; Sphingomonas sp. MEA3-1 contig00040, whole genome shotgun
sequence; 873296160; NZ_LECE01000040.1
3490; Sphingomonas sp. MEA3-1 contig00071, whole genome shotgun
sequence; 873296295; NZ_LECE01000071.1
3491; Bacillus sp. 220_BSPC 1447_75439_1072255, whole genome shotgun
sequence; 880954155; NZ_JVPL01000109.1
3492; Bacillus sp. 522_BSPC 2470_72498_1083579_594_{——}. . ._522_, whole
genome shotgun sequence; 880997761; NZ_JVDT01000118.1
3493; Nostoc sp. PCC 7107, complete genome; 427705465; NC_019676.1
3494; Streptomyces decoyicus strain NRRL ISP-5087
P056_Doro1_scaffold78, whole genome shotgun sequence; 662133033;
NZ_KL570321.1
3495; Streptomyces varsoviensis strain NRRL B-3589 contig2.1, whole
genome shotgun sequence; 664348063; NZ_JOFN01000002.1
3496; Scytonema tolypothrichoides VB-61278 scaffold_6, whole genome
shotgun sequence; 890002594; NZ_JXCA01000005.1
3497; Erythrobacter atlanticus strain s21-N3, complete genome; 890444402;
NZ_CP011310.1
3498; Sphingobium yanoikuyae strain SHJ scaffold2, whole genome shotgun
sequence; 893711333; NZ_KQ235984.1
3499; Sphingobium yanoikuyae strain SHJ scaffold12, whole genome shotgun
sequence; 893711343; NZ_KQ235994.1
3500; Sphingobium yanoikuyae strain SHJ scaffold33, whole genome shotgun
sequence; 893711364; NZ_KQ236015.1
3501; Sphingobium yanoikuyae strain SHJ scaffold47, whole genome shotgun
sequence; 893711378; NZ_KQ236029.1
3502; Stenotrophomonas maltophilia strain 544_SMAL
1161_223966_2976806_599_{——}. . ._882_, whole genome shotgun sequence;
896492362; NZ_JVCU01000107.1
3503; Stenotrophomonas maltophilia strain 517_SMAL
472_405557_4951990_20_{——}. . ._115_, whole genome shotgun sequence;
896506125; NZ_JVDZ01000045.1
3504; Stenotrophomonas maltophilia strain 131_SMAL
1126_236170_8501292_717_{——}. . ._1018_, whole genome shotgun sequence;
896520167; NZ_JVUI01000038.1
3505; Stenotrophomonas maltophilia strain 419_SMAL
707_128228_1961615_4_{——}642_{——}523_, whole genome shotgun sequence;
896535166; NZ_JVHW01000017.1
3506; Stenotrophomonas maltophilia strain 179_SMAL
631_468538_7028045_522_{——}. . ._127_, whole genome shotgun sequence;
896555871; NZ_JVRD01000056.1
3507; Stenotrophomonas maltophilia strain 951_SMAL 71_125859_2268311,
whole genome shotgun sequence; 896567682; NZ_JUMH01000022.1
3508; Stenotrophomonas maltophilia strain 22_SMAL
361_494818_13518495_244_{——}194_{——}203_, whole genome shotgun sequence;
896599318; NZ_JVPM01000019.1
3509; Streptococcus pseudopneumoniae strain 445_SPSE
347_91401_2272315_318_{——}. . ._319_, whole genome shotgun sequence;
896667361; NZ_JVGV01000030.1
3510; Streptomyces sp. SBT349 scaffold307_size9018, whole genome
shotgun sequence; 898301838; NZ_LAVK01000307.1
3511; Kitasatospora sp. MY 5-36 Contig_703_, whole genome shotgun
sequence; 902792184; NZ_LFVW01000692.1
3512; Streptomyces caatingaensis strain CMAA 1322 contig02, whole genome
shotgun sequence; 906344334; NZ_LFXA01000002.1
3513; Streptomyces caatingaensis strain CMAA 1322 contig02, whole genome
shotgun sequence; 906344334; NZ_LFXA01000002.1
3514; Streptomyces caatingaensis strain CMAA 1322 contig07, whole genome
shotgun sequence; 906344339; NZ_LFXA01000007.1
3515; Streptomyces caatingaensis strain CMAA 1322 contig09, whole genome
shotgun sequence; 906344341; NZ_LFXA01000009.1
3516; Xanthomonas arboricola 3004 contig00003, whole genome shotgun
sequence; 640500871; NZ_AZQY01000003.1
3517; Candidatus Halobonum tyrrellensis G22 contig00002, whole genome
shotgun sequence; 557371823; NZ_ASGZ01000002.1
3518; Streptomyces wadayamensis strain A23 LGO_A23_AS7_CO0257,
whole genome shotgun sequence; 910050821; NZ_JHDU01000034.1
3519; Bacillus weihenstephanensis strain JAS 83/3 Bw_JAS-
83/3_contig00005, whole genome shotgun sequence; 910095435;
NZ_JNLY01000005.1
3520; Silvibacterium bohemicum strain S15 contig_3, whole genome shotgun
sequence; 910257956; NZ_LBHJ01000003.1
3521; Silvibacterium bohemicum strain S15 contig_3, whole genome shotgun
sequence; 910257956; NZ_LBHJ01000003.1
3522; Silvibacterium bohemicum strain S15 contig_30, whole genome shotgun
sequence; 910257973; NZ_LBHJ01000020.1
3523; Streptococcus pneumoniae strain 37, whole genome shotgun sequence;
912648153; NZ_CKHR01000004.1
3524; Streptococcus pneumoniae strain 37, whole genome shotgun sequence;
912676034; NZ_CMPZ01000004.1
3525; Streptomyces fradiae strain ATCC 19609 contig0008, whole genome
shotgun sequence; 759752221; NZ_JNAD01000008.1
3526; Streptomyces sp. CNS654 CD02DRAFT_scaffold00023.23_C, whole
genome shotgun sequence; 695856316; NZ_JNLT01000024.1
3527; Streptomyces griseus subsp. rhodochrous strain NRRL B-2931 contig3.1,
whole genome shotgun sequence; 664191782; NZ_JOFE01000003.1
3528; Streptomyces sp. NRRL F-2202 contig25.1, whole genome shotgun
sequence; 695860443; NZ_JOIH01000025.1
3529; Streptomyces purpeochromogenes strain NRRL B-3012 contig5.1,
whole genome shotgun sequence; 663242068; NZ_JODK01000005.1
3530; Streptomyces griseus subsp. rhodochrous strain NRRL B-2932 contig37.1,
whole genome shotgun sequence; 664207653; NZ_JOFF01000037.1
3531; Streptomyces sp. NRRL F-5702 contig3.1, whole genome shotgun
sequence; 664537198; NZ_JOHD01000003.1
3532; Streptomyces albus subsp. albus strain NRRL B-2445 contig1.1, whole
genome shotgun sequence; 664084661; NZ_JOED01000001.1
3533; Streptomyces baarnensis strain NRRL B-2842 P144_Doro1_scaffold6,
whole genome shotgun sequence; 662129456; NZ_KL573544.1
3534; Streptomyces sp. NRRL F-3218 contig19.1, whole genome shotgun
sequence; 664170107; NZ_JOIP01000019.1
3535; Streptomyces albus subsp. albus strain NRRL B-2445 contig1.1, whole
genome shotgun sequence; 664084661; NZ_JOED01000001.1
3536; Streptomyces albus subsp. albus strain NRRL B-16041 contig26.1,
whole genome shotgun sequence; 695869320; NZ_JNWW01000026.1
3537; Streptomyces albus subsp. albus strain NRRL B-16041 contig28.1,
whole genome shotgun sequence; 695870063; NZ_JNWW01000028.1
3538; Streptomyces peucetius strain NRRL WC-3868 contig49.1, whole
genome shotgun sequence; 665671804; NZ_JOCK01000052.1
3539; Erythrobacter citreus LAMA 915 Contig13, whole genome shotgun
sequence; 914607448; NZ_JYNE01000028.1
3540; Bacillus flexus strain Riq5 contig_32, whole genome shotgun sequence;
914730676; NZ_LFQJ01000032.1
3541; Xylanimonas cellulosilytica DSM 15894, complete genome;
269954810; NC_013530.1
3542; Streptomyces sp. Mg1, complete genome; 847063800; NZ_CP011664.1
3543; Streptomyces sviceus ATCC 29083 chromosome, whole genome
shotgun sequence; 297196766; NZ_CM000951.1
3544; Burkholderia pseudomallei strain MSHR0169 Contig_2, whole genome
shotgun sequence; 915621003; NZ_LGKL01000002.1
3545; Burkholderia pseudomallei strain E25, whole genome shotgun sequence;
915671105; NZ_CSLP01000001.1
3546; Streptomyces xinghaiensis S187 contig_1763_1, whole genome shotgun
sequence; 485454803; NZ_AFRP01001656.1
3547; Streptomyces sp. W007 contig00241, whole genome shotgun sequence;
365866490; NZ_AGSW01000226.1
3548; Streptomyces violaceusniger Tu 4113, complete genome; 345007964;
NC_015957.1
3549; Streptomyces mobaraensis NBRC 13819 = DSM 40847 contig024,
whole genome shotgun sequence; 458977979; NZ_AORZ01000024.1
3550; Streptomyces mobaraensis NBRC 13819 = DSM 40847 contig079,
whole genome shotgun sequence; 458984960; NZ_AORZ01000079.1
3551; Actinokineospora enzanensis DSM 44649
C503DRAFT_scaffold00014.14, whole genome shotgun sequence;
484005069; NZ_KB894416.1
3552; Streptomyces sp. FXJ7.023 Contig10, whole genome shotgun sequence;
510871397; NZ_APIV01000010.1
3553; Nocardia transvalensis NBRC 15921, whole genome shotgun sequence;
485125031; NZ_BAGL01000055.1
3554; Caulobacter sp. URHA0033 H963DRAFT_scaffold00023.23_C, whole
genome shotgun sequence; 654573246; NZ_AUEO01000025.1
3555; Gloeobacter kilaueensis JS1, complete genome; 554634310;
NC_022600.1
3556; Actinomadura oligospora ATCC 43269 P696DRAFT_scaffold00008.8_C,
whole genome shotgun sequence; 651281457; NZ_JADG01000010.1
3557; Actinomadura oligospora ATCC 43269 P696DRAFT_scaffold00008.8_C,
whole genome shotgun sequence; 651281457; NZ_JADG01000010.1
3558; Streptomyces sp. Tu 6176 scaffold00003, whole genome shotgun
sequence; 740044478; NZ_KK106990.1
3559; Sphingomonas paucimobilis strain EPA505 contig000027, whole
genome shotgun sequence; 739630357; NZ_JFYY01000027.1
3560; Paenibacillus sp. UNC217MF BP95DRAFT_scaffold00011.11_C,
whole genome shotgun sequence; 655084059; NZ_JMLT01000016.1
3561; Hyphomonas chukchiensis strain BH-BN04-4 contig29, whole genome
shotgun sequence; 736736050; NZ_AWFG01000029.1
3562; Fusobacterium necrophorum BFTR-2 contig0075, whole genome
shotgun sequence; 737951550; NZ_JAAG01000075.1
3563; Streptomyces sp. NRRL F-5917 contig68.1, whole genome shotgun
sequence; 663414324; NZ_JOHQ01000068.1
3564; Streptomyces sp. NRRL F-5639 contig31.1, whole genome shotgun
sequence; 664512262; NZ_JOGK01000031.1
3565; Streptomyces sp. NRRL F-5639 contig75.1, whole genome shotgun
sequence; 664515060; NZ_JOGK01000075.1
3566; Streptomyces megasporus strain NRRL B-16372 contig19.1, whole
genome shotgun sequence; 671525382; NZ_JODL01000019.1
3567; Streptomyces albus subsp. albus strain NRRL B-1811 contig32.1, whole
genome shotgun sequence; 665618015; NZ_JODR01000032.1
3568; Streptomyces albus subsp. albus strain NRRL B-1811 contig49.1, whole
genome shotgun sequence; 665618560; NZ_JODR01000049.1
3569; Streptomyces griseus subsp. griseus strain NRRL WC-3480 contig2.1,
whole genome shotgun sequence; 664166765; NZ_JOBR01000002.1
3570; Streptomyces griseorubens strain JSD-1 scaffold1, whole genome
shotgun sequence; 739792456; NZ_KL503830.1
3571; Streptomyces achromogenes subsp. achromogenes strain NRRL B-2120 contig2.1,
whole genome shotgun sequence; 664063830; NZ_JODT01000002.1
3572; Nocardia sp. NRRL WC-3656 contig2.1, whole genome shotgun
sequence; 663737675; NZ_JOJF01000002.1
3573; Streptomyces sp. NRRL S-337 contig31.1, whole genome shotgun
sequence; 664275807; NZ_JOIX01000031.1
3574; Streptomyces sp. NRRL S-337 contig41.1, whole genome shotgun
sequence; 664277815; NZ_JOIX01000041.1
3575; Streptomyces albus subsp. albus strain NRRL B-2362 contig48.1, whole
genome shotgun sequence; 739761647; NZ_JODZ01000048.1
3576; Streptomyces ruber strain NRRL ISP-5378 contig2.1, whole genome
shotgun sequence; 665674644; NZ_JOAQ01000002.1
3577; Streptomyces lavenduligriseus strain NRRL ISP-5487 contig2.1, whole
genome shotgun sequence; 664244706; NZ_JOBD01000002.1
3578; Streptomyces sp. NRRL S-920 contig36.1, whole genome shotgun
sequence; 664256887; NZ_JODF01000036.1
3579; Streptomyces sp. NRRL S-1448 contig134.1, whole genome shotgun
sequence; 663421576; NZ_JOGE01000134.1
3580; Streptomyces bicolor strain NRRL B-3897 contig42.1, whole genome
shotgun sequence; 671498318; NZ_JOFR01000042.1
3581; Streptomyces sp. NRRL WC-3719 contig52.1, whole genome shotgun
sequence; 665530468; NZ_JOCD01000052.1
3582; Streptomyces sp. NRRL WC-3719 contig152.1, whole genome shotgun
sequence; 665536304; NZ_JOCD01000152.1
3583; Streptomyces sp. NRRL WC-3641 P206_Doro1_scaffold18, whole
genome shotgun sequence; 664607641; NZ_KL579016.1
3584; Streptomyces sp. NRRL B-1347 contig19.1, whole genome shotgun
sequence; 664141438; NZ_JOJM01000019.1
3585; Streptomyces toyocaensis strain NRRL 15009 contig00064, whole
genome shotgun sequence; 740092143; NZ_JFCB01000064.1
3586; Streptomyces natalensis strain NRRL B-5314 P055_Doro1_scaffold13,
whole genome shotgun sequence; 662108422; NZ_KL570019.1
3587; Sphingobium yanoikuyae strain B1 contig000002, whole genome
shotgun sequence; 739661773; NZ_JGVR01000002.1
3588; Kibdelosporangium aridum subsp. largum strain NRRL B-24462 contig91.5,
whole genome shotgun sequence; 703243990; NZ_JNYM01001430.1
3589; Streptomyces ruber strain NRRL ISP-5378 contig2.1, whole genome
shotgun sequence; 665674644; NZ_JOAQ01000002.1
3590; Kutzneria albida DSM 43870, complete genome; 754862786;
NZ_CP007155.1
3591; Streptomyces aurantiacus JA 4570 Seq28, whole genome shotgun
sequence; 514916412; NZ_AOPZ01000028.1
3592; Rothia dentocariosa strain C6B contig_5, whole genome shotgun
sequence; 739372122; NZ_JQHE01000003.1
3593; Xanthomonas cannabis pv. phaseoli strain Nyagatare scf_52938_7,
whole genome shotgun sequence; 835885587; NZ_KN265462.1
3594; Novosphingobium malaysiense strain MUSC 273 Contig9, whole
genome shotgun sequence; 746241774; NZ_JTDI01000009.1
3595; Novosphingobium subterraneum strain DSM 12447 NJ75_contig000028,
whole genome shotgun sequence; 746290581; NZ_JRVC01000028.1
3596; Jeotgalibacillus malaysiensis strain D5 chromosome, complete genome;
749182744; NZ_CP009416.1
3597; Microcystis panniformis FACHB-1757, complete genome; 917764592;
NZ_CP011339.1
3598; Streptomyces sp. 769, complete genome; 749181963; NZ_CP003987.1
3599; Actinoplanes sp. SE50/110, complete genome; 386845069;
NC_017803.1
3600; Salinarimonas rosea DSM 21201 G407DRAFT_scaffold00021.21_C,
whole genome shotgun sequence; 655990125; NZ_AUBC01000024.1
3601; Methanobacterium arcticum strain M2 EI99DRAFT_scaffold00005.5_C,
whole genome shotgun sequence; 851140085; NZ_JQKN01000008.1
3602; Allokutzneria albata strain NRRL B-24461 contig22.1, whole genome
shotgun sequence; 663596322; NZ_JOEF01000022.1
3603; Streptomyces olivaceus strain NRRL B-3009 contig20.1, whole genome
shotgun sequence; 664523889; NZ_JOFH01000020.1
3604; Streptomyces sp. NRRL S-118 P205_Doro1_scaffold2, whole genome
shotgun sequence; 664556736; NZ_KL591003.1
3605; Streptomyces sp. NRRL S-118 P205_Doro1_scaffold34, whole genome
shotgun sequence; 664565137; NZ_KL591029.1
3606; Streptomyces luteus strain TRM 45540 Scaffold1, whole genome
shotgun sequence; 759659849; NZ_KN039946.1
3607; Nonomuraea Candida strain NRRL B-24552 contig8.1, whole genome
shotgun sequence; 759934284; NZ_JOAG01000009.1
3608; Nonomuraea Candida strain NRRL B-24552 contig19.1, whole genome
shotgun sequence; 759941310; NZ_JOAG01000020.1
3609; Nonomuraea Candida strain NRRL B-24552 contig27.1, whole genome
shotgun sequence; 759944049; NZ_JOAG01000029.1
3610; Nonomuraea Candida strain NRRL B-24552 contig28.1, whole genome
shotgun sequence; 759944490; NZ_JOAG01000030.1
3611; Nonomuraea Candida strain NRRL B-24552 contig42.1, whole genome
shotgun sequence; 759948103; NZ_JOAG01000045.1
3612; Streptacidiphilus melanogenes strain NBRC 103184, whole genome
shotgun sequence; 755032408; NZ_BBPP01000024.1
3613; Streptacidiphilus anmyonensis strain NBRC 103185, whole genome
shotgun sequence; 755077919; NZ_BBPQ01000048.1
3614; Streptomyces nodosus strain ATCC 14899 genome; 759739811;
NZ_CP009313.1
3615; Kibdelosporangium sp. MJ126-NF4, whole genome shotgun sequence;
754819815; NZ_CDME01000002.1
3616; Streptomyces albus strain DSM 41398, complete genome; 749658562;
NZ_CP010519.1
3617; Novosphingobium sp. P6W scaffold17, whole genome shotgun
sequence; 763097360; NZ_JXZE01000017.1
3618; Magnetospirillum gryphiswaldense MSR-1 v2, complete genome;
568144401; NC_023065.1
3619; Methanobacterium formicicum genome assembly DSM1535,
chromosome: chrI; 851114167; NZ_LN515531.1
3620; Streptomyces sp. NRRL B-1568 contig-76, whole genome shotgun
sequence; 799161588; NZ_JZWZ01000076.1
3621; Streptomyces rubellomurinus subsp. indigofems strain ATCC 31304 contig-55,
whole genome shotgun sequence; 783374270; NZ_JZKG01000056.1
3622; Paenibacillus dauci strain H9 Scaffold3, whole genome shotgun
sequence; 808064534; NZ_KQ040798.1
3623; Allosalinactinospora lopnorensis strain CA15-2 contig00053, whole
genome shotgun sequence; 815864238; NZ_LAJC01000053.1
3624; Jiangella alkaliphila strain KCTC 19222 Scaffold1, whole genome
shotgun sequence; 820820518; NZ_KQ061219.1
3625; Streptomyces natalensis ATCC 27448 Scaffold_46, whole genome
shotgun sequence; 764442321; NZ_JRKI01000041.1
3626; Sphingomonas parapaucimobilis NBRC 15100 BBPI01000030, whole
genome shotgun sequence; 755134941; NZ_BBPI01000030.1
3627; Streptomyces avicenniae strain NRRL B-24776 contig3.1, whole
genome shotgun sequence; 919531973; NZ_JOEK01000003.1
3628; Streptomyces celluloflavus strain NRRL B-2493 contig27.1, whole
genome shotgun sequence; 919546534; NZ_JOEL01000027.1
3629; Streptomyces celluloflavus strain NRRL B-2493 contig60.1, whole
genome shotgun sequence; 919546651; NZ_JOEL01000060.1
3630; Streptomyces celluloflavus strain NRRL B-2493 contig66.1, whole
genome shotgun sequence; 919546672; NZ_JOEL01000066.1
3631; Sphingomonas sp. Y57 scaffold74, whole genome shotgun sequence;
826051019; NZ_LDES01000074.1
3632; Xanthomonas arboricola pv. juglandis strain Xaj 417 genome;
920673152; NZ_CP012251.1
3633; Xanthomonas campestris strain CFSAN033089 contig_46, whole
genome shotgun sequence; 920684790; NZ_LHBW01000046.1
3634; Streptomyces sp. Mg1 supercont1.100, whole genome shotgun
sequence; 254387191; NZ_DS570483.1
3635; Streptomyces sp. HNS054 contig28, whole genome shotgun sequence;
860547590; NZ_LDZX01000028.1
3636; Streptomyces ahygroscopicus subsp. wuyiensis strain CK-15 contig3,
whole genome shotgun sequence; 921220646; NZ_JXYI02000059.1
3637; Paenibacillus peoriae strain HS311, complete genome; 922052336;
NZ_CP011512.1
3638; Paenibacillus sp. FJAT-27812 scaffold_0, whole genome shotgun
sequence; 922780240; NZ_LIGH01000001.1
3639; Stenotrophomonas maltophilia strain ISMMS2R, complete genome;
923060045; NZ_CP011306.1
3640; Stenotrophomonas maltophilia strain ISMMS3, complete genome;
923067758; NZ_CP011010.1
3641; Hapalosiphon sp. MRB220 contig_91, whole genome shotgun
sequence; 923076229; NZ_LIRN01000111.1
3642; Bacillus sp. FJAT-18019 super1, whole genome shotgun sequence;
924371245; NZ_LITP01000001.1
3643; Stenotrophomonas maltophilia strain B4 contig779, whole genome
shotgun sequence; 924516300; NZ_LDVR01000003.1
3644; Bacillus sp. FJAT-21352 Scaffold1, whole genome shotgun sequence;
924654439; NZ_LIUS01000003.1
3645; Sphingopyxis sp. 113P3, complete genome; 924898949;
NZ_CP009452.1
3646; Sphingopyxis sp. 113P3, complete genome; 924898949;
NZ_CP009452.1
3647; Streptomyces sp. CFMR 7 strain CFMR-7, complete genome;
924911621; NZ_CP011522.1
3648; Bacillus gobiensis strain FJAT-4402 chromosome; 926268043;
NZ_CP012600.1
3649; Streptomyces sp. MMG1522 P406contig11.1, whole genome shotgun
sequence; 926270045; NZ_LGDF01000013.1
3650; Nocardiopsis sp. NRRL B-16309 P441contig5.1, whole genome
shotgun sequence; 926283036; NZ_LGEC01000103.1
3651; Streptomyces sp. NRRL F-2295 P395contig79.1, whole genome
shotgun sequence; 926288193; NZ_LGCY01000146.1
3652; Streptomyces sp. XY431 P412contig111.1, whole genome shotgun
sequence; 926317398; NZ_LGDO01000015.1
3653; Streptomyces sp. NRRL F-6492 P446contig3.1, whole genome shotgun
sequence; 926315769; NZ_LGEG01000211.1
3654; Streptomyces sp. NRRL B-1140 P439contig15.1, whole genome
shotgun sequence; 926344107; NZ_LGEA01000058.1
3655; Streptomyces sp. NRRL B-1140 P439contig32.1, whole genome
shotgun sequence; 926344331; NZ_LGEA01000105.1
3656; Streptomyces sp. NRRL F-5755 P309contig48.1, whole genome
shotgun sequence; 926371517; NZ_LGCW01000271.1
3657; Streptomyces sp. NRRL F-5755 P309contig50.1, whole genome
shotgun sequence; 926371520; NZ_LGCW01000274.1
3658; Streptomyces sp. NRRL F-5755 P309contig7.1, whole genome shotgun
sequence; 926371541; NZ_LGCW01000295.1
3659; Saccharothrix sp. NRRL B-16348 P442contig71.1, whole genome
shotgun sequence; 926395199; NZ_LGED01000246.1
3660; Streptomyces sp. WM6378 P402contig63.1, whole genome shotgun
sequence; 926403453; NZ_LGDD01000321.1
3661; Streptomyces sp. WM6378 P402contig63.1, whole genome shotgun
sequence; 926403453; NZ_LGDD01000321.1
3662; Nocardia sp. NRRL S-836 P437contig3.1b, whole genome shotgun
sequence; 926412094; NZ_LGDY01000103.1
3663; Nocardia sp. NRRL S-836 P437contig39.1, whole genome shotgun
sequence; 926412104; NZ_LGDY01000113.1
3664; Paenibacillus sp. A59 contig_353, whole genome shotgun sequence;
927084730; NZ_LITU01000050.1
3665; Paenibacillus sp. A59 contig_416, whole genome shotgun sequence;
927084736; NZ_LITU01000056.1
3666; Streptomyces sp. XY332 P409contig34.1, whole genome shotgun
sequence; 927093145; NZ_LGHN01000166.1
3667; Streptomyces rimosus subsp. rimosus strain NRRL WC-3898 P259contig86.1,
whole genome shotgun sequence; 927279089; NZ_LGCU01000353.1
3668; Streptomyces rimosus subsp. pseudoverticillatus strain NRRL WC-3896
P270contig51.1, whole genome shotgun sequence; 927292651; NZ_LGCV01000382.1
3669; Streptomyces rimosus subsp. pseudoverticillatus strain NRRL WC-3896
P270contig8.1, whole genome shotgun sequence; 927292684; NZ_LGCV01000415.1
3670; Aneurinibacillus migulanus strain Nagano E1 contig_36, whole genome
shotgun sequence; 928874573; NZ_LIXL01000208.1
3671; Streptomyces chattanoogensis strain NRRL ISP-5002 ISP5002contig8.1,
whole genome shotgun sequence; 928897585; NZ_LGKG01000196.1
3672; Streptomyces chattanoogensis strain NRRL ISP-5002 ISP5002contig9.1,
whole genome shotgun sequence; 928897596; NZ_LGKG01000207.1
3673; Streptomyces sp. NRRL F-6602 F6602contig54.1, whole genome
shotgun sequence; 928910033; NZ_LGKH01004848.1
3674; Ideonella sakaiensis strain 201-F6, whole genome shotgun sequence;
928998724; NZ_BBYR01000007.1
3675; Ideonella sakaiensis strain 201-F6, whole genome shotgun sequence;
928998800; NZ_BBYR01000083.1
3676; Bacillus sp. FJAT-28004 scaffold_2, whole genome shotgun sequence;
929005248; NZ_LGHP01000003.1
3677; Novosphingobium sp. AAP1 AAP1Contigs7, whole genome shotgun
sequence; 930029075; NZ_LJHO01000007.1
3678; Novosphingobium sp. AAP1 AAP1Contigs9, whole genome shotgun
sequence; 930029077; NZ_LJHO01000009.1
3679; Stenotrophomonas maltophilia strain OC194 contig_98, whole genome
shotgun sequence; 930169273; NZ_LJJH01000098.1
3680; Actinobacteria bacterium OK074 ctg60, whole genome shotgun
sequence; 930473294; NZ_LJCV01000275.1
3681; Actinobacteria bacterium OK006 ctg112, whole genome shotgun
sequence; 930490730; NZ_LJCU01000014.1
3682; Actinobacteria bacterium OK006 ctg96, whole genome shotgun
sequence; 930491003; NZ_LJCU01000287.1
3683; Kibdelosporangium phytohabitans strain KLBMP1111, complete
genome; 931609467; NZ_CP012752.1
3684; Streptococcus pneumoniae strain PT8082 isolate E3GXY, whole
genome shotgun sequence; 935445269; NZ_CIEC02000098.1
3685; Paenibacillus solani strain FJAT-22460 super3, whole genome shotgun
sequence; 935460965; NZ_LIUT01000006.1
3686; Novosphingobium sp. ST904 contig_104, whole genome shotgun
sequence; 935540718; NZ_LGJH01000063.1
3687; Citromicrobium sp. RCC1878 contig2, whole genome shotgun
sequence; 936191447; NZ_LBLZ01000002.1
3688; Frankia sp. R43 contig001, whole genome shotgun sequence;
937182893; NZ_LFCW01000001.1
3689; Sphingopyxis macrogoltabida strain EY-1, complete genome;
937372567; NZ_CP012700.1
3690; Sphingopyxis macrogoltabida strain EY-1, complete genome;
937372567; NZ_CP012700.1
3691; Xanthomonas arboricola strain CITA 44 CITA_44_contig_26, whole
genome shotgun sequence; 937505789; NZ_LJGM01000026.1
3692; Stenotrophomonas acidaminiphila strain ZAC14D2_NAIMI4_2,
complete genome; 938883590; NZ_CP012900.1
3693; Sphingopyxis macrogoltabida strain 203, complete genome; 938956730;
NZ_CP009429.1
3694; Sphingopyxis macrogoltabida strain 203, complete genome; 938956730;
NZ_CP009429.1
3695; Sphingopyxis macrogoltabida strain 203 plasmid, complete sequence;
938956814; NZ_CP009430.1
3696; Cellulosilyticum ruminicola JCM 14822, whole genome shotgun
sequence; 938965628; NZ_BBCG01000065.1
3697; Brevundimonas sp. DS20, complete genome; 938989745; NZ_CP012897.1
3698; Brevundimonas sp. DS20, complete genome; 938989745; NZ_CP012897.1
3699; Paenibacillus sp. GD6, whole genome shotgun sequence; 939708098;
NZ_LN831198.1
3700; Paenibacillus sp. GD6, whole genome shotgun sequence; 939708105;
NZ_LN831205.1
3701; Alicyclobacillus ferrooxydans strain TC-34 contig_22, whole genome
shotgun sequence; 940346731; NZ_LJCO01000107.1
3702; Xanthomonas sp. Mitacek01 contig_17, whole genome shotgun
sequence; 941965142; NZ_LKIT01000002.1
3703; Streptomyces bingchenggensis BCW-1, complete genome; 374982757;
NC_016582.1
3704; Streptomyces pactum strain ACT12 scaffold1, whole genome shotgun
sequence; 943388237; NZ_LIQD01000001.1
3705; Streptomyces flocculus strain NRRL B-2465 B2465_contig_205, whole
genome shotgun sequence; 943674269; NZ_LIQO01000205.1
3706; Streptomyces aurantiacus strain NRRL ISP-5412 ISP-5412_contig_138,
whole genome shotgun sequence; 943881150; NZ_LIPP01000138.1
3707; Streptomyces graminilatus strain NRRL B-59124 B59124_contig_7,
whole genome shotgun sequence; 943897669; NZ_LIQQ01000007.1
3708; Streptomyces alboniger strain NRRL B-1832 B-1832_contig_37, whole
genome shotgun sequence; 943898694; NZ_LIQN01000037.1
3709; Streptomyces alboniger strain NRRL B-1832 B-1832_contig_384,
whole genome shotgun sequence; 943899498; NZ_LIQN01000384.1
3710; Streptomyces kanamyceticus strain NRRL B-2535 B-2535_contig_122,
whole genome shotgun sequence; 943922224; NZ_LIQU01000122.1
3711; Streptomyces kanamyceticus strain NRRL B-2535 B-2535_contig_247,
whole genome shotgun sequence; 943922567; NZ_LIQU01000247.1
3712; Streptomyces luridiscabiei strain NRRL B-24455 B24455_contig_315,
whole genome shotgun sequence; 943927948; NZ_LIQV01000315.1
3713; Streptomyces atriruber strain NRRL B-24165 contig_124, whole
genome shotgun sequence; 943949281; NZ_LIPN01000124.1
3714; Streptomyces hirsutus strain NRRL B-2713 B2713_contig_57, whole
genome shotgun sequence; 944005810; NZ_LIQT01000057.1
3715; Streptomyces aureus strain NRRL B-2808 contig_171, whole genome
shotgun sequence; 944012845; NZ_LIPQ01000171.1
3716; Streptomyces prasinus strain NRRL B-12521 B12521_contig_230,
whole genome shotgun sequence; 944020089; NZ_LIPR01000230.1
3717; Streptomyces phaeochromogenes strain NRRL B-1248 B-1248_contig_126,
whole genome shotgun sequence; 944029528; NZ_LIQZ01000126.1
3718; Streptomyces prasinus strain NRRL B-2712 B2712_contig_323, whole
genome shotgun sequence; 944410649; NZ_LIRH01000323.1
3719; Streptomyces prasinopilosus strain NRRL B-2711 B2711_contig_370,
whole genome shotgun sequence; 944415035; NZ_LIRG01000370.1
3720; Streptomyces torulosus strain NRRL B-3889 B-3889_contig_18, whole
genome shotgun sequence; 944495433; NZ_LIRK01000018.1
3721; Frankia alni str. ACN14A chromosome, complete sequence;
111219505; NC_008278.1
3722; Frankia sp. CpI1-S FF36_scaffold_9.10, whole genome shotgun
sequence; 768715243; NZ_JYFN01000010.1
3723; Sphingomonas sp. Leaf20 contig_1, whole genome shotgun sequence;
947349881; NZ_LMKN01000001.1
3724; Paenibacillus sp. Leaf72 contig_6, whole genome shotgun sequence;
947378267; NZ_LMLV01000032.1
3725; Sphingomonas sp. Leaf230 contig_4, whole genome shotgun sequence;
947401208; NZ_LMKW01000010.1
3726; Sanguibacter sp. Leaf3 contig_2, whole genome shotgun sequence;
947472882; NZ_LMRH01000002.1
3727; Aeromicrobium sp. Root344 contig_1, whole genome shotgun
sequence; 947552260; NZ_LMDH01000001.1
3728; Sphingopyxis sp. Root1497 contig_3, whole genome shotgun sequence;
947689975; NZ_LMGF01000003.1
3729; Sphingomonas sp. Root1294 contig_7, whole genome shotgun
sequence; 947890193; NZ_LMEJ01000014.1
3730; Sphingomonas sp. Root720 contig_7, whole genome shotgun sequence;
947704642; NZ_LMID01000015.1
3731; Sphingomonas sp. Root720 contig_8, whole genome shotgun sequence;
947704650; NZ_LMID01000016.1
3732; Sphingomonas sp. Root710 contig_1, whole genome shotgun sequence;
947721816; NZ_LMIB01000001.1
3733; Sphingomonas sp. Root1294 contig_7, whole genome shotgun
sequence; 947890193; NZ_LMEJ01000014.1
3734; Mesorhizobium sp. Root172 contig_2, whole genome shotgun sequence;
947919015; NZ_LMHP01000012.1
3735; Mesorhizobium sp. Root102 contig_3, whole genome shotgun sequence;
947937119; NZ_LMCP01000023.1
3736; Paenibacillus sp. Soil750 contig_1, whole genome shotgun sequence;
947966412; NZ_LMSD01000001.1
3737; Paenibacillus sp. Soil522 contig_3, whole genome shotgun sequence;
947983982; NZ_LMRV01000044.1
3738; Paenibacillus sp. Soil522 contig_3, whole genome shotgun sequence;
947983982; NZ_LMRV01000044.1
3739; Paenibacillus sp. Root52 contig_3, whole genome shotgun sequence;
948045460; NZ_LMFO01000023.1
3740; Enterococcus faecalis ATCC 29212 contig24, whole genome shotgun
sequence; 401673929; ALOD01000024.1
3741; Mesorhizobium sp. Root695 contig_1, whole genome shotgun sequence;
950019035; NZ_LMHO01000001.1
3742; Bacillus sp. Soil768D1 contig_5, whole genome shotgun sequence;
950170460; NZ_LMTA01000046.1
3743; Paenibacillus sp. Root444D2 contig_4, whole genome shotgun
sequence; 950271971; NZ_LMEO01000034.1
3744; Paenibacillus sp. Soil766 contig_32, whole genome shotgun sequence;
950280827; NZ_LMSJ01000026.1
3745; Streptococcus pneumoniae strain type strain: N, whole genome shotgun
sequence; 950938054; NZ_CIHL01000007.1
3746; Streptomyces sp. Root1310 contig_5, whole genome shotgun sequence;
951121600; NZ_LMEQ01000031.1
3747; Bacillus muralis strain DSM 16288 Scaffold4, whole genome shotgun
sequence; 951610263; NZ_LMBV01000004.1
3748; Streptomyces sp. MBT76 scaffold_2, whole genome shotgun sequence;
953813788; NZ_LNBE01000002.1
3749; Streptomyces sp. MBT76 scaffold_3, whole genome shotgun sequence;
953813789; NZ_LNBE01000003.1
3750; Streptomyces sp. MBT76 scaffold_4, whole genome shotgun sequence;
953813790; NZ_LNBE01000004.1
3751; Clostridium butyricum strain KNU-L09 chromosome 1, complete
sequence; 959868240; NZ_CP013252.1
3752; Clostridium butyricum strain NEC8, whole genome shotgun sequence;
960334134; NZ_CBYK010000003.1
3753; Gorillibacterium sp. SN4, whole genome shotgun sequence; 960412751;
NZ_LN881722.1
3754; Thalassobius activus strain CECT 5114, whole genome shotgun
sequence; 960424655; NZ_CYUE01000025.1
3755; Microbacterium testaceum strain NS283 contig_37, whole genome
shotgun sequence; 969836538; NZ_LDRU01000037.1
3756; Microbacterium testaceum strain NS206 contig_27, whole genome
shotgun sequence; 969912012; NZ_LDRS01000027.1
3757; Microbacterium testaceum strain NS183 contig_65, whole genome
shotgun sequence; 969919061; NZ_LDRR01000065.1
3758; Paenibacillus jamilae strain NS115 contig_27, whole genome shotgun
sequence; 970428876; NZ_LDRX01000027.1
3759; Sphingopyxis sp. H050 H050_contig000006, whole genome shotgun
sequence; 970555001; NZ_LNRZ01000006.1
3760; Paenibacillus polymyxa strain KF-1 scaffold00001, whole genome
shotgun sequence; 970574347; NZ_LNZF01000001.1
3761; Luteimonas abyssi strain XH031 Scaffold1, whole genome shotgun
sequence; 970579907; NZ_KQ759763.1

TABLE 5
Exemplary Lasso RRE
Lasso RRE Peptide No: #; Species of Origin; GI#; Accession#

3762; Geobacter uraniireducens Rf4, complete genome; 148262085;
NC_009483.1
3763; Sphingomonas wittichii RW1, complete genome; 148552929;
NC_009511.1
3764; Sanguibacter keddieii DSM 10542, complete genome; 269793358;
NC_013521.1
3765; Xylanimonas cellulosilytica DSM 15894, complete genome;
269954810; NC_013530.1
3766; Spirosoma linguale DSM 74, complete genome; 283814236;
CP001769.1
3767; Streptomyces bingchenggensis BCW-1, complete genome; 374982757;
NC_016582.1
3768; Streptomyces bingchenggensis BCW-1, complete genome; 374982757;
NC_016582.1
3769; Gallionella capsiferriformans ES-2, complete genome; 302877245;
NC_014394.1
3770; Mycobacterium sinense strain JDM601, complete genome; 333988640;
NC_015576.1
3771; Streptomyces violaceusniger Tu 4113, complete genome; 345007964;
NC_015957.1
3772; Rhodospirillum rubrum F11, complete genome; 386348020;
NC_017584.1
3773; Actinoplanes sp. SE50/110, complete genome; 386845069;
NC_017803.1
3774; Emticicia oligotrophica DSM 17448, complete genome; 408671769;
NC_018748.1
3775; Tistrella mobilis KA081020-065 plasmid pTM1, complete sequence;
442559580; NC_017957.2
3776; Bacillus thuringiensis MC28, complete genome; 407703236;
NC_018693.1
3777; Nostoc sp. PCC 7107, complete genome; 427705465; NC_019676.1
3778; Synechococcus sp. PCC 6312, complete genome; 427711179;
NC_019680.1
3779; Stanieria cyanosphaera PCC 7437, complete genome; 428267688;
CP003653.1
3780; Desulfocapsa sulfexigens DSM 10523, complete genome; 451945650;
NC_020304.1
3781; Xanthomonas citri pv. punicae str. LMG 859, whole genome shotgun
sequence; 390991205; NZ_CAGJ01000031.1
3782; Streptomyces fulvissimus DSM 40593, complete genome; 488607535;
NC_021177.1
3783; Streptomyces rapamycinicus NRRL 5491 genome; 521353217;
CP006567.1
3784; Gloeobacter kilaueensis JS1, complete genome; 554634310;
NC_022600.1
3785; Gloeobacter kilaueensis JS1, complete genome; 554634310;
NC_022600.1
3786; Kutzneria albida strain NRRL B-24060 contig305.1, whole genome
shotgun sequence; 662161093; NZ_JNYH01000515.1
3787; Kutzneria albida strain NRRL B-24060 contig305.1, whole genome
shotgun sequence; 662161093; NZ_JNYH01000515.1
3788; Mesorhizobium huakuii 7653R genome; 657121522; CP006581.1
3789; Sphingopyxis fribergensis strain Kp5.2, complete genome; 749188513;
NZ_CP009122.1
3790; Amycolatopsis lurida NRRL 2430, complete genome; 755908329;
CP007219.1
3791; Streptomyces lydicus A02, complete genome; 822214995;
NZ_CP007699.1
3792; Streptomyces lydicus A02, complete genome; 822214995;
NZ_CP007699.1
3793; Uncultured bacterium clone AZ25P121 genomic sequence; 818476494;
KP274854.1
3794; Streptomyces sp. PBH53 genome; 852460626; CP011799.1
3795; Streptomyces sp. PBH53 genome; 852460626; CP011799.1
3796; Streptomyces sp. PBH53 genome; 852460626; CP011799.1
3797; Bifidobacterium longum subsp. infantis strain BT1, complete genome;
927296881; CP010411.1
3798; Nostoc piscinale CENA21 genome; 930349143; CP012036.1
3799; Paenibacillus sp. 32O-W, complete genome; 961447255; CP013653.1
3800; Streptomyces avermitilis MA-4680 = NBRC 14893, complete genome;
162960844; NC_003155.4
3801; Streptomyces avermitilis MA-4680 = NBRC 14893, complete genome;
162960844; NC_003155.4
3802; Kitasatospora setae KM-6054 DNA, complete genome; 357386972;
NC_016109.1
3803; Rhodococcus jostii lariatin biosynthetic gene cluster (larA, larB, larC,
larD, larE), complete cds; 380356103; AB593691.1
3804; Pseudomonas sp. St29 DNA, complete genome; 771846103;
AP014628.1
3805; Pseudomonas sp. St29 DNA, complete genome; 771846103;
AP014628.1
3806; Fischerella sp. NIES-3754 DNA, complete genome; 965684975;
AP017305.1
3807; Magnetospirillum gryphiswaldense MSR-1, WORKING DRAFT
SEQUENCE, 373 unordered pieces; 144897097; CU459003.1
3808; Streptococcus suis 98HAH33, complete genome; 145690656;
CP000408.1
3809; Salinibacter ruber M8 chromosome, complete genome; 294505815;
NC_014032.1
3810; Enterococcus faecalis strain P9-1 scaffold484.1, whole genome shotgun
sequence; 949763393; NZ_LKGS01000484.1
3811; Saccharothrix espanaensis DSM 44229 complete genome; 433601838;
NC_019673.1
3812; Roseburia sp. CAG: 197 WGS project CBBL01000000 data, contig,
whole genome shotgun sequence; 524261006; CBBL010000225.1
3813; Clostridium sp. CAG: 221 WGS project CBDC01000000 data, contig,
whole genome shotgun sequence; 524362382; CBDC010000065.1
3814; Clostridium sp. CAG: 411 WGS project CBIY01000000 data, contig,
whole genome shotgun sequence; 524742306; CBIY010000075.1
3815; Roseburia sp. CAG: 100 WGS project CBKV01000000 data, contig,
whole genome shotgun sequence; 524842500; CBKV010000277.1
3816; Mesorhizobium plurifarium, whole genome shotgun sequence;
751292755; NZ_CCNE01000004.1
3817; Kibdelosporangium sp. MJ126-NF4, whole genome shotgun sequence;
754819815; NZ_CDME01000002.1
3818; Kibdelosporangium sp. MJ126-NF4 genome assembly High
quaKibdelosporangium sp. MJ126-NF4, scaffold BPA_8, whole genome
shotgun sequence; 747653426; CDME0100001
3819; Methanobacterium formicicum genome assembly isolate Mb9,
chromosome: I; 952971377; LN734822.1
3820; Streptococcus pneumoniae strain type strain: N, whole genome shotgun
sequence; 950938054; NZ_CIHL01000007.1
3821; Streptococcus pneumoniae strain type strain: N, whole genome shotgun
sequence; 950938054; NZ_CIHL01000007.1
3822; Streptococcus pneumoniae strain type strain: N, whole genome shotgun
sequence; 950938054; NZ_CIHL01000007.1
3823; Bacillus cereus genome assembly Bacillus JRS4, contig contig000025,
whole genome shotgun sequence; 924092470; CYHM01000025.1
3824; Pedobacter sp. BAL39 1103467000492, whole genome shotgun
sequence; 149277373; NZ_ABCM01000005.1
3825; Streptomyces sviceus ATCC 29083 chromosome, whole genome
shotgun sequence; 297196766; NZ_CM000951.1
3826; Streptomyces pristinaespiralis ATCC 25486 chromosome, whole
genome shotgun sequence; 297189896; NZ_CM000950.1
3827; Enterococcus faecalis strain P9-1 scaffold484.1, whole genome shotgun
sequence; 949763393; NZ_LKGS01000484.1
3828; Enterococcus faecalis strain P9-1 scaffold484.1, whole genome shotgun
sequence; 949763393; NZ_LKGS01000484.1
3829; Streptomyces sp. CNS654 CD02DRAFT_scaffold00023.23_C, whole
genome shotgun sequence; 695856316; NZ_JNLT01000024.1
3830; Streptococcus vestibularis F0396 ctg1126932565723, whole genome
shotgun sequence; 311100538; AEKO01000007.1
3831; Ruminococcus albus 8 contig00035, whole genome shotgun sequence;
325680876; NZ_ADKM02000123.1
3832; Streptomyces sp. W007 contig00293, whole genome shotgun sequence;
365867746; NZ_AGSW01000272.1
3833; Streptomyces auratus AGR0001 Scaffold1_85, whole genome shotgun
sequence; 396995461; AJGV01000085.1
3834; Actinomyces naeslundii str. Howell 279 ctg1130888818142, whole
genome shotgun sequence; 399903251; ALJK01000024.1
3835; Enterococcus faecalis strain P9-1 scaffold484.1, whole genome shotgun
sequence; 949763393; NZ_LKGS01000484.1
3836; Amycolatopsis decaplanina DSM 44594 Contig0055, whole genome
shotgun sequence; 458848256; NZ_AOHO01000055.1
3837; Streptomyces mobaraensis NBRC 13819 = DSM 40847 contig024,
whole genome shotgun sequence; 458977979; NZ_AORZ01000024.1
3838; Enterococcus faecalis strain P9-1 scaffold484.1, whole genome shotgun
sequence; 949763393; NZ_LKGS01000484.1
3839; Enterococcus faecalis strain P9-1 scaffold484.1, whole genome shotgun
sequence; 949763393; NZ_LKGS01000484.1
3840; Streptomyces aurantiacus JA 4570 Seq28, whole genome shotgun
sequence; 514916412; NZ_AOPZ01000028.1
3841; Streptomyces aurantiacus JA 4570 Seq17, whole genome shotgun
sequence; 514916021; NZ_AOPZ01000017.1
3842; Enterococcus faecalis strain P9-1 scaffold484.1, whole genome shotgun
sequence; 949763393; NZ_LKGS01000484.1
3843; Paenibacillus alvei A6-6i-x PAAL66ix_14, whole genome shotgun
sequence; 528200987; ATMS01000061.1
3844; Dehalobacter sp. UNSWDHB Contig_139, whole genome shotgun
sequence; 544905305; NZ_AUUR01000139.1
3845; Actinobaculum sp. oral taxon 183 str. F0552 A_P1HMPREF0043-
1.0_Cont15.2, whole genome shotgun sequence; 541473965;
AWSB01000041.1
3846; Actinobaculum sp. oral taxon 183 str. F0552 A_P1HMPREF0043-
1.0_Cont1.1, whole genome shotgun sequence; 541476958;
AWSB01000006.1
3847; Propionibacterium acidifaciens F0233 ctg1127964738299, whole
genome shotgun sequence; 544249812; ACVN02000045.1
3848; Rubidibacter lacunae KORDI 51-2 KR51_contig00121, whole genome
shotgun sequence; 550281965; NZ_ASSJ01000070.1
3849; Rothia aeria F0184 R_aeriaHMPREF0742-1.0_Cont136.4, whole
genome shotgun sequence; 551695014; AXZG01000035.1
3850; Candidatus Halobonum tyrrellensis G22 contig00002, whole genome
shotgun sequence; 557371823; NZ_ASGZ01000002.1
3851; Streptomyces niveus NCIMB 11891 contig00003, whole genome
shotgun sequence; 558542923; AWQW01000003.1
3852; Frankia sp. Thr ThrDRAFT_scaffold_28.29, whole genome shotgun
sequence; 602262270; JENI01000029.1
3853; Clostridium butyricum DORA_1 Q607_CBUC00058, whole genome
shotgun sequence; 566226100; AZLX01000058.1
3854; Streptococcus sp. DORA_10 Q617_SPSC00257, whole genome
shotgun sequence; 566231608; AZMH01000257.1
3855; Candidatus Entotheonella factor TSY1_contig00913, whole genome
shotgun sequence; 575408569; AZHW01000959.1
3856; Streptomyces sp. CNS654 CD02DRAFT_scaffold00023.23_C, whole
genome shotgun sequence; 695856316; NZ_JNLT01000024.1
3857; Frankia sp. Thr ThrDRAFT_scaffold_28.29, whole genome shotgun
sequence; 602262270; JENI01000029.1
3858; Bacillus akibai JCM 9157, whole genome shotgun sequence;
737696658; NZ_BAUV01000025.1
3859; Bacillus boroniphilus JCM 21738 DNA, contig: contig_6, whole
genome shotgun sequence; 571146044; BAUW01000006.1
3860; Gracilibacillus boraciitolerans JCM 21714 DNA, contig: contig_30,
whole genome shotgun sequence; 575082509; BAVS01000030.1
3861; Streptomyces griseorubens strain JSD-1 contig143, whole genome
shotgun sequence; 657284919; JJMG01000143.1
3862; Frankia sp. CeD CEDDRAFT_scaffold_22.23, whole genome shotgun
sequence; 737947180; NZ_JPGU01000023.1
3863; Frankia sp. Thr ThrDRAFT_scaffold_28.29, whole genome shotgun
sequence; 602262270; JENI01000029.1
3864; Streptomyces sp. JS01 contig2, whole genome shotgun sequence;
695871554; NZ_JPWW01000002.1
3865; Rothia dentocariosa strain C6B contig_5, whole genome shotgun
sequence; 739372122; NZ_JQHE01000003.1
3866; Candidatus Thiomargarita nelsonii isolate Hydrate Ridge contig_1164,
whole genome shotgun sequence; 723288710; JSZA01001164.1
3867; Streptomyces globisporus C-1027 Scaffold24_1, whole genome shotgun
sequence; 41065119; NZ_AJUO01000171.1
3868; Lechevalieria aerocolonigenes strain NRRL B-16140 contig11.3, whole
genome shotgun sequence; 772744565; NZ_JYJG01000059.1
3869; Desulfobulbaceae bacterium BRH_c16a BRHa_1001515, whole
genome shotgun sequence; 780791108; LADS01000058.1
3870; Peptococcaceae bacterium BRH_c4b BRHa_1001357, whole genome
shotgun sequence; 780813318; LADO01000010.1
3871; Streptomyces rubellomurinus subsp. indigofems strain ATCC 31304
contig-55, whole genome shotgun sequence; 783374270;
NZ_JZKG01000056.1
3872; Streptomyces sp. NRRL S-444 contig322.4, whole genome shotgun
sequence; 797049078; JZWX01001028.1
3873; Streptomyces sp. NRRL B-1568 contig-76, whole genome shotgun
sequence; 799161588; NZ_JZWZ01000076.1
3874; Streptomyces caatingaensis strain CMAA 1322 contig02, whole genome
shotgun sequence; 906344334; NZ_LFXA01000002.1
3875; Paenibacillus polymyxa strain YUPP-8 scaffold32, whole genome
shotgun sequence; 924434005; LIYK01000027.1
3876; Streptomyces rimosus subsp. rimosus strain NRRL B-16073 contig48.1,
whole genome shotgun sequence; 696497741; NZ_JNWX01000048.1
3877; Streptomyces rimosus subsp. rimosus ATCC 10970 contig00333, whole
genome shotgun sequence; 441178796; NZ_ANSJ01000259.1
3878; Streptomyces rimosus subsp. rimosus strain NRRL B-16073 contig48.1,
whole genome shotgun sequence; 696497741; NZ_JNWX01000048.1
3879; Streptomyces rimosus subsp. rimosus strain NRRL WC-3869
P248contig20.1, whole genome shotgun sequence; 925322461;
LGCQ01000113.1
3880; Streptomyces rimosus subsp. rimosus ATCC 10970 contig00333, whole
genome shotgun sequence; 441178796; NZ_ANSJ01000259.1
3881; Streptomyces rimosus subsp. rimosus ATCC 10970 contig00333, whole
genome shotgun sequence; 441178796; NZ_ANSJ01000259.1
3882; Streptomyces rimosus subsp. rimosus strain NRRL B-16073 contig48.1,
whole genome shotgun sequence; 696497741; NZ_JNWX01000048.1
3883; Streptomyces rimosus subsp. rimosus strain NRRL B-2660 contigl24.1,
whole genome shotgun sequence; 664066234; NZ_JOES01000124.1
3884; Streptomyces sp. NRRL F-5755 P309contig50.1, whole genome
shotgun sequence; 926371520; NZ_LGCW01000274.1
3885; Streptomyces sp. NRRL F-5755 P309contig48.1, whole genome
shotgun sequence; 926371517; NZ_LGCW01000271.1
3886; Streptomyces sp. NRRL S-444 contig322.4, whole genome shotgun
sequence; 797049078; JZWX01001028.1
3887; Actinobacteria bacterium OK006 ctg96, whole genome shotgun
sequence; 930491003; NZ_LJCU01000287.1
3888; Actinobacteria bacterium OK074 ctg60, whole genome shotgun
sequence; 930473294; NZ_LJCV01000275.1
3889; Betaproteobacteria bacterium SG8_39 WOR_8-12_2589, whole
genome shotgun sequence; 931421682; LJTQ01000030.1
3890; Candidate division BRC1 bacterium SM23_51 WORSMTZ_10094,
whole genome shotgun sequence; 931536013; LJUL01000022.1
3891; Bacillus vietnamensis strain UCD-SED5 scaffold_15, whole genome
shotgun sequence; 933903534; LIXZ01000017.1
3892; Erythrobacteraceae bacterium HL-111 ITZY_scaf_51, whole genome
shotgun sequence; 938259025; LJSW01000006.1
3893; Halomonas sp. HL-93 ITZY_scaf_415, whole genome shotgun
sequence; 938285459; LJST01000237.1
3894; Paenibacillus sp. Soil724D2 contig_11, whole genome shotgun
sequence; 946400391; LMRY01000003.1
3895; Paenibacillus sp. Root444D2 contig_4, whole genome shotgun
sequence; 950271971; NZ_LMEO01000034.1
3896; Streptomyces silvensis strain ATCC 53525
53525_Assembly_Contig_22, whole genome shotgun sequence; 970361514;
LOCL01000028.1
3897; Bacillus mycoides strain Flugge 10206 DJ94.contig-100_16, whole
genome shotgun sequence; 727343482; NZ_JMQD01000030.1
3898; Bacillus cereus BAG3X2-1 supercont1.1, whole genome shotgun
sequence; 423416528; NZ_JH791923.1
3899; Bacillus thuringiensis MC28, complete genome; 407703236;
NC_018693.1
3900; Bacillus cereus VD131 acrHi-supercont2.9, whole genome shotgun
sequence; 507037581; NZ_KB976660.1
3901; Bacillus cereus Rock4-18 chromosome, whole genome shotgun
sequence; 238801487; NZ_CM000735.1
3902; Bacillus cereus AH1271 chromosome, whole genome shotgun
sequence; 238801491; NZ_CM000739.1
3903; Bacillus cereus Rock3-44 chromosome, whole genome shotgun
sequence; 238801485; NZ_CM000733.1
3904; Bacillus cereus VD115 supercont1.1, whole genome shotgun sequence;
423614674; NZ_JH792165.1
3905; Bacillus sp. UMTAT18 contig000011, whole genome shotgun
sequence; 806951735;NZ_JSFD01000011.1
3906; Bacillus cereus BAG5X2-1 supercont1.1, whole genome shotgun
sequence; 423456860; NZ_JH791975.1
3907; Streptococcus pneumoniae strain type strain: N, whole genome shotgun
sequence; 950938054; NZ_CIHL01000007.1
3908; Bacillus census VD142 actaa-supercont2.2, whole genome shotgun
sequence; 514340871; NZ_KE150045.1
3909; Bacillus census BAG6O-2 supercont1.1, whole genome shotgun
sequence; 423468694; NZ_JH804628.1
3910; Bacillus mycoides FSLH7-687 Contig052, whole genome shotgun
sequence; 727271768; NZ_ASPY01000052.1
3911; Bacillus census HuA2-9 acqVt-supercont1.1, whole genome shotgun
sequence; 507020427; NZ_KB976152.1
3912; Bacillus census HuA4-10 supercont1.1, whole genome shotgun
sequence; 423520617; NZ_JH792148.1
3913; Bacillus census MC67 supercont1.2, whole genome shotgun sequence;
423557538; NZ_JH792114.1
3914; Bacillus census AH621 chromosome, whole genome shotgun sequence;
238801471; NZ_CM000719.1
3915; Bacillus census VD107 supercont1.1, whole genome shotgun sequence;
423609285; NZ_JH792232.1
3916; Bacillus census VDM034 supercont1.1, whole genome shotgun
sequence; 423666303; NZ_JH791809.1
3917; Bacillus cereus BAG5X1-1 supercont1.1, whole genome shotgun
sequence; 423451256; NZ_JH791996.1
3918; Enterococcus faecalis strain P9-1 scaffold484.1, whole genome shotgun
sequence; 949763393; NZ_LKGS01000484.1
3919; Clostridium butyricum 5521 gcontig_1106103650482, whole genome
shotgun sequence; 182420360; NZ_ABDT01000120.2
3920; Rhodobacter sphaeroides WS8N chromosome chrI, whole genome
shotgun sequence; 332561612; NZ_CM001161.1
3921; Methylosinus trichosporium OB3b MettrDRAFT_Contig_106_C, whole
genome shotgun sequence; 639846426; NZ_ADVE02000001.1
3922; Streptomyces clavuligerus ATCC 27064 supercont1.55, whole genome
shotgun sequence; 254392242; NZ_DS570678.1
3923; Streptomyces rimosus subsp. rimosus strain NRRL B-16073 contig48.1,
whole genome shotgun sequence; 696497741; NZ_JNWX01000048.1
3924; Streptomyces rimosus subsp. rimosus ATCC 10970 contig00333, whole
genome shotgun sequence; 441178796; NZ_ANSJ01000259.1
3925; Streptomyces viridochromogenes DSM 40736 supercont1.1, whole
genome shotgun sequence; 224581107; NZ_GG657757.1
3926; Streptomyces viridochromogenes DSM 40736 supercont1.1, whole
genome shotgun sequence; 224581107; NZ_GG657757.1
3927; Methanobacterium formicicum DSM 3637 Contig04, whole genome
shotgun sequence; 408381849; NZ_AMPO01000004.1
3928; Methanobacterium formicicum DSM 3637 Contig04, whole genome
shotgun sequence; 408381849; NZ_AMPO01000004.1
3929; Sphingobium yanoikuyae strain SHJ scaffold2, whole genome shotgun
sequence; 893711333; NZ_KQ235984.1
3930; Streptomyces mobaraensis NBRC 13819 = DSM 40847 contig024,
whole genome shotgun sequence; 458977979; NZ_AORZ01000024.1
3931; Streptomyces mobaraensis NBRC 13819 = DSM 40847 contig079,
whole genome shotgun sequence; 458984960; NZ_AORZ01000079.1
3932; Amycolatopsis azurea DSM 43854 contig60, whole genome shotgun
sequence; 451338568; NZ_ANMG01000060.1
3933; Streptomyces pristinaespiralis ATCC 25486 chromosome, whole
genome shotgun sequence; 297189896; NZ_CM000950.1
3934; Xanthomonas citri pv. punicae str. LMG 859, whole genome shotgun
sequence; 390991205; NZ_CAGJ01000031.1
3935; Streptomyces sp. CNS654 CD02DRAFT_scaffold00023.23_C, whole
genome shotgun sequence; 695856316; NZ_JNLT01000024.1
3936; Mesorhizobium amorphae CCNWGS0123 contig00204, whole genome
shotgun sequence; 357028583; NZ_AGSN01000187.1
3937; Leptolyngbya sp. PCC 7375 Lepto7375DRAFT_LPA.5, whole genome
shotgun sequence; 427415532; NZ_JH993797.1
3938; Streptomyces auratus AGR0001 Scaffold1, whole genome shotgun
sequence; 398790069; NZ_JH725387.1
3939; Streptomyces auratus AGR0001 Scaffold1_85, whole genome shotgun
sequence; 396995461; AJGV01000085.1
3940; Paenibacillus dendritiformis C454 PDENDC1000064, whole genome
shotgun sequence; 374605177; NZ_AHKH01000064.1
3941; Halosimplex carlsbadense 2-9-1 contig_4, whole genome shotgun
sequence; 448406329: NZ_AOIU01000004.1
3942; Amycolatopsis decaplanina DSM 44594 Contig0055, whole genome
shotgun sequence; 458848256; NZ_AOHO01000055.1
3943; Fictibacillus macauensis ZFHKF-1 Contig20, whole genome shotgun
sequence; 392955666; NZ_AKKV01000020.1
3944; Streptomyces sviceus ATCC 29083 chromosome, whole genome
shotgun sequence; 297196766; NZ_CM000951.1
3945; Paenibacillus sp. Aloe-11 GW8_15, whole genome shotgun sequence;
375307420; NZ_JH601049.1
3946; Streptomyces sp. W007 contig00293, whole genome shotgun sequence;
365867746; NZ_AGSW01000272.1
3947; Frankia saprophytica strain CN3 FrCN3DRAFT_FCB.2, whole genome
shotgun sequence; 652876473; NZ_KI912267.1
3948; Desulfosporosinus youngiae DSM 17734 chromosome, whole genome
shotgun sequence; 374578721; NZ_CM001441.1
3949; Moorea producens 3L scf52054, whole genome shotgun sequence;
332710503; NZ_GL890955.1
3950; Pedobacter sp. BAL39 1103467000500, whole genome shotgun
sequence; 149277003; NZ_ABCM01000004.1
3951; Pedobacter sp. BAL39 1103467000492, whole genome shotgun
sequence; 149277373; NZ_ABCM01000005.1
3952; Sulfurovum sp. AR contig00449, whole genome shotgun sequence;
386284588; NZ_AJLE01000006.1
3953; Mucilaginibacter paludis DSM 18603 chromosome, whole genome
shotgun sequence; 373951708; NZ_CM001403.1
3954; Magnetospirillum caucaseum strain SO-1 contig00006, whole genome
shotgun sequence; 458904467; NZ_AONQ01000006.1
3955; Moorea producens 3L scf52052, whole genome shotgun sequence;
332710285; NZ_GL890953.1
3956; Cecembia lonarensis LW9 contig000133, whole genome shotgun
sequence; 406663945; NZ_AMGM01000133.1
3957; Actinomyces sp. oral taxon 848 str. F0332 Scfld0, whole genome
shotgun sequence; 260447107; NZ_GG703879.1
3958; Actinomyces sp. oral taxon 848 str. F0332 Scfld0, whole genome
shotgun sequence; 260447107; NZ_GG703879.1
3959; Streptomyces ipomoeae 91-03 gcontig_1108499710267, whole genome
shotgun sequence; 429195484; NZ_AEJC01000118.1
3960; Streptomyces ipomoeae 91-03 gcontig_1108499715961, whole genome
shotgun sequence; 429196334; NZ_AEJC01000180.1
3961; Frankia sp. QA3 chromosome, whole genome shotgun sequence;
392941286; NZ_CM001489.1
3962; Fischerella thermalis PCC 7521 contig00099, whole genome shotgun
sequence; 484076371; NZ_AJLL01000098.1
3963; Rhodobacter sp. AKP1 contig19, whole genome shotgun sequence;
429208285; NZ_ANFS01000019.1
3964; Streptomyces chartreusis NRRL 12338 12338_Doro1_scaffold19,
whole genome shotgun sequence; 381200190; NZ_JH164855.1
3965; Streptomyces globisporus C-1027 Scaffold24_1, whole genome shotgun
sequence; 410651191; NZ_AJUO01000171.1
3966; Sphingobium yanoikuyae XLDN2-5 contig000029, whole genome
shotgun sequence; 378759075; NZ_AFXE01000029.1
3967; Paenibacillus peoriae KCTC 3763 contig9, whole genome shotgun
sequence; 389822526; NZ_AGFX01000048.1
3968; Citromicrobium sp. JLT1363 contig00009, whole genome shotgun
sequence; 341575924; NZ_AEUE01000009.1
3969; Acaryochloris sp. CCMEE 5410 contig00232, whole genome shotgun
sequence; 359367134; NZ_AFEJ01000154.1
3970; Pseudomonas extremaustralis 14-3 substr. 14-3b strain 14-3
contig00001, whole genome shotgun sequence; 394743069;
NZ_AHIP01000001.1
3971; Lunatimonas lonarensis strain AK24 S14_contig_18, whole genome
shotgun sequence; 499123840; NZ_AQHR01000021.1
3972; Mesorhizobium japonicum R7A MesloDRAFT_Scaffold1.1, whole
genome shotgun sequence; 696358903; NZ_KI632510.1
3973; Legionella pneumophila subsp. pneumophila ATCC 43290, complete
genome; 378775961; NC_016811.1
3974; Methylococcus capsulatus str. Texas = ATCC 19069 strain Texas
contig0129, whole genome shotgun sequence; 483090991;
NZ_AMCE01000064.1
3975; Thermobifida fusca TM51 contig028, whole genome shotgun sequence;
510814910; NZ_AOSG01000028.1
3976; Rhodospirillum rubrum F11, complete genome; 386348020;
NC_017584.1
3977; Rhodospirillum rubrum F11, complete genome; 386348020;
NC_017584.1
3978; Rhodospirillum rubrum F11, complete genome; 386348020;
NC_017584.1
3979; Hahella chejuensis KCTC 2396, complete genome; 83642913;
NC_007645.1
3980; Frankia sp. Thr ThrDRAFT_scaffold_28.29, whole genome shotgun
sequence; 602262270; JENI01000029.1
3981; Novosphingobium aromaticivorans DSM 12444, complete genome;
87198026; NC_007794.1
3982; Roseobacter denitrificans OCh 114, complete genome; 110677421;
NC_008209.1
3983; Pelobacter propionicus DSM 2379, complete genome; 118578449;
NC_008609.1
3984; Psychromonas ingrahamii 37, complete genome; 119943794;
NC_008709.1
3985; Rhodobacter sphaeroides ATCC 17029 chromosome 1, complete
sequence; 126460778; NC_009049.1
3986; Rhodobacter sphaeroides ATCC 17025, complete genome; 146276058;
NC_009428.1
3987; Geobacter uraniireducens Rf4, complete genome; 148262085;
NC_009483.1
3988; Sphingomonas wittichii RW1, complete genome; 148552929;
NC_009511.1
3989; Sulfurovum sp. NBC37-1 genomic DNA, complete genome;
152991597; NC_009663.1
3990; Acaryochloris marina MBIC11017, complete genome; 158333233;
NC_009925.1
3991; Bacillus weihenstephanensis KBAB4, complete genome; 163938013;
NC_010184.1
3992; Bifidobacterium longum subsp. infantis ATCC 15697, complete
genome; 213690928; NC_011593.1
3993; Cyanothece sp. PCC 7425, complete genome; 220905643;
NC_011884.1
3994; Streptococcus suis 98HAH33, complete genome; 145690656;
CP000408.1
3995; Chitinophaga pinensis DSM 2588, complete genome; 256419057;
NC_013132.1
3996; Rhodothermus marinus DSM 4252, complete genome; 268315578;
NC_013501.1
3997; Sanguibacter keddieii DSM 10542, complete genome; 269793358;
NC_013521.1
3998; Thermobaculum terrenum ATCC BAA-798 chromosome 1, complete
sequence; 269925123; NC_013525.1
3999; Thermobaculum terrenum ATCC BAA-798 chromosome 2, complete
sequence; 269838913; NC_013526.1
4000; Xylanimonas cellulosilytica DSM 15894, complete genome;
269954810; NC_013530.1
4001; Salinibacter ruber M8 chromosome, complete genome; 294505815;
NC_014032.1
4002; Salinibacter ruber M8 chromosome, complete genome; 294505815;
NC_014032.1
4003; Legionella pneumophila 2300/99 Alcoy, complete genome; 296105497;
NC_014125.1
4004; Amycolatopsis mediterranei S699, complete genome; 384145136;
NC_017186.1
4005; Butyrivibrio proteoclasticus B316 chromosome 1, complete sequence;
302669374; NC_014387.1
4006; Gallionella capsiferriformans ES-2, complete genome; 302877245;
NC_014394.1
4007; Paenibacillus polymyxa E681, complete genome; 864439741;
NC_014483.2
4008; Paenibacillus polymyxa 1-43 S143_contig00221, whole genome
shotgun sequence; 647225094; NZ_ASRZ01000173.1
4009; Mesorhizobium ciceri CMG6 MescicDRAFT_scaffold_1.2_C, whole
genome shotgun sequence; 639162053; NZ_AWZS01000002.1
4010; Terriglobus saanensis SP1PR4, complete genome; 320105246;
NC_014963.1
4011; Syntrophobotulus glycolicus DSM 8271, complete genome; 325288201;
NC_015172.1
4012; Methanobacterium lacus strain AL-21, complete genome; 325957759;
NC_015216.1
4013; Marinomonas mediterranea MMB-1, complete genome; 326793322;
NC_015276.1
4014; Desulfobacca acetoxidans DSM 11109, complete genome; 328951746;
NC_015388.1
4015; Methylomonas methanica MC09, complete genome; 333981747;
NC_015572.1
4016; Methylomonas methanica MC09, complete genome; 333981747;
NC_015572.1
4017; Methanobacterium paludis strain SWAN1, complete genome;
333986242; NC_015574.1
4018; Mycobacterium sinense strain JDM601, complete genome; 333988640;
NC_015576.1
4019; Frankia coriariae strain BMG5.1 scaffold41.42, whole genome shotgun
sequence; 827465632; NZ_JWIO01000042.1
4020; Halopiger xanaduensis SH-6 plasmid pHALXA01, complete genome;
336251750; NC_015658.1
4021; Mesorhizobium opportunistum WSM2075, complete genome;
337264537;NC_015675.1
4022; Runella slithyformis DSM 19594, complete genome; 338209545;
NC_015703.1
4023; Roseobacter litoralis Och 149, complete genome; 339501577;
NC_015730.1
4024; Streptomyces violaceusniger Tu 4113 plasmid pSTRVI01, complete
sequence; 345007457; NC_015951.1
4025; Streptomyces violaceusniger Tu 4113, complete genome; 345007964;
NC_015957.1
4026; Rhodothermus marinus SG0.5JP17-172, complete genome; 345301888;
NC_015966.1
4027; Chloracidobacterium thermophilum B chromosome 1, complete
sequence; 347753732; NC_016024.1
4028; Kitasatospora setae KM-6054 DNA, complete genome; 357386972;
NC_016109.1
4029; Streptomyces bingchenggensis BCW-1, complete genome; 374982757;
NC_016582.1
4030; Desulfosporosinus orientis DSM 765, complete genome; 374992780;
NC_016584.1
4031; Desulfosporosinus orientis DSM 765, complete genome; 374992780;
NC_016584.1
4032; Paenibacillus terrae HPL-003, complete genome; 374319880;
NC_016641.1
4033; Bacillus megaterium WSH-002. complete genome; 384044176;
NC_017138.1
4034; Francisella cf. novicida 3523, complete genome; 387823583;
NC_017449.1
4035; Streptomyces cattleya str. NRRL 8057 main chromosome, complete
genome; 357397620; NC_016111.1
4036; Streptococcus salivarius JIM8777 complete genome; 387783149;
NC_017595.1
4037; Actinoplanes sp. SE50/110, complete genome; 386845069;
NC_017803.1
4038; Tistrella mobilis KA081020-065 plasmid pTM1, complete sequence;
442559580; NC_017957.2
4039; Tistrella mobilis KA081020-065 plasmid pTM3, complete sequence;
389874236; NC_017958.1
4040; Tistrella mobilis KA081020-065 plasmid pTM3, complete sequence;
389874236; NC_017958.1
4041; Legionella pneumophila subsp. pneumophila str. Lorraine chromosome,
complete genome; 397662556; NC_018139.1
4042; Nocardiopsis sp. TP-A0876 strain NBRC 110039, whole genome
shotgun sequence; 754924215; NZ_BAZE01000001.1
4043; Emticicia oligotrophica DSM 17448, complete genome; 408671769;
NC_018748.1
4044; Saccharothrix espanaensis DSM 44229 complete genome; 433601838;
NC_019673.1
4045; Saccharothrix espanaensis DSM 44229 complete genome; 433601838;
NC_019673.1
4046; Nostoc sp. PCC 7107, complete genome; 427705465; NC_019676.1
4047; Nostoc sp. PCC 7107, complete genome; 427705465; NC_019676.1
4048; Rivularia sp. PCC 7116, complete genome; 427733619; NC_019678.1
4049; Rivularia sp. PCC 7116, complete genome; 427733619; NC_019678.1
4050; Synechococcus sp. PCC 6312, complete genome; 427711179;
NC_019680.1
4051; Synechococcus sp. PCC 6312, complete genome; 427711179;
NC_019680.1
4052; Nostoc sp. PCC 7524, complete genome; 427727289; NC_019684.1
4053; Calothrix sp. PCC 6303, complete genome; 428296779; NC_019751.1
4054; Crinalium epipsammum PCC 9333, complete genome; 428303693;
NC_019753.1
4055; Thermobacillus composti KWC4, complete genome; 430748349;
NC_019897.1
4056; Mesorhizobium sp. LNHC220B00 scaffold0002, whole genome
shotgun sequence; 563576979; NZ_AYWS01000002.1
4057; Bacillus sp. 1NLA3E, complete genome; 488570484; NC_021171.1
4058; Streptomyces davawensis strain JCM 4913 complete genome;
471319476; NC_020504.1
4059; Streptomyces davawensis strain JCM 4913 complete genome;
471319476; NC_020504.1
4060; Desulfotomaculum acetoxidans DSM 771, complete genome;
258513366; NC_013216.1
4061; Desulfotomaculum acetoxidans DSM 771, complete genome;
258513366; NC_013216.1
4062; Actinosynnema minim DSM 43827, complete genome; 256374160;
NC_013093.1
4063; Bacillus cereus BAG2O-3 acfXF-supercont1.1, whole genome shotgun
sequence; 507017505; NZ_KB976530.1
4064; Bacillus cereus HuA3-9 acqVv-supercont1.4, whole genome shotgun
sequence; 507024338; NZ_KB976146.1
4065; Bacillus cereus VD118 acrHo-supercont1.9, whole genome shotgun
sequence; 507035131; NZ_KB976800.1
4066; Bacillus cereus VDM006 acrHb-supercont1.1, whole genome shotgun
sequence; 507060269; NZ_KB976864.1
4067; Bacillus cereus VDM019 achrj-supercont1.2, whole genome shotgun
sequence; 507056808; NZ_KB976199.1
4068; Bacillus cereus VDM053 acrGS-supcrcont1.7, whole genome shotgun
sequence; 507060152; NZ_KB976714.1
4069; Halomonas anticariensis FP35 = DSM 16096 strain FP35 Scaffold1,
whole genome shotgun sequence; 514429123; NZ_KE332377.1
4070; Streptomyces sp. NRRL F-5917 contig68.1, whole genome shotgun
sequence; 663414324; NZ_JOHQ01000068.1
4071; Streptomyces aurantiacus JA 4570 Seq17, whole genome shotgun
sequence; 514916021; NZ_AOPZ01000017.1
4072; Streptomyces aurantiacus JA 4570 Seq63, whole genome shotgun
sequence; 514917321; NZ_AOPZ01000063.1
4073; Streptomyces aurantiacus JA 4570 Seq109, whole genome shotgun
sequence; 514918665; NZ_AOPZ01000109.1
4074; Paenibacillus polymyxa OSY-DF Contig136, whole genome shotgun
sequence; 484036841; NZ_AIPP01000136.1
4075; Fischerella muscicola SAG 1427-1 = PCC 73103 contig00215, whole
genome shotgun sequence; 484073367; NZ_AJLJ01000207.1
4076; Fischerella muscicola PCC 7414 contig00109, whole genome shotgun
sequence; 484075173; NZ_AJLK01000109.1
4077; Fischerella muscicola PCC 7414 contig00153, whole genome shotgun
sequence; 484075372; NZ_AJLK01000153.1
4078; Pedobacter arcticus A12 Scaffold2, whole genome shotgun sequence;
484345004; NZ_JH947126.1
4079; Leptolyngbya boryana PCC 6306 LepboDRAFT_LPC.1, whole
genome shotgun sequence; 482909028; NZ_KB731324.1
4080; Mastigocladus laminosus UU774 scaffold_22, whole genome shotgun
sequence; 764671177; NZ_JXIJ01000139.1
4081; Fischerella sp. PCC 9339 PCC9339DRAFT_scaffold1.1, whole genome
shotgun sequence; 482909394; NZ_JH992898.1
4082; Lactococcus garvieae Tac2 Tac2Contig_33, whole genome shotgun
sequence; 483258918; NZ_AMFE01000033.1
4083; Paenisporosarcina sp. TG-14 111.TG14.1_1, whole genome shotgun
sequence; 483299154; NZ_AMGD01000001.1
4084; Paenibacillus sp. ICGEB2008 Contig_7, whole genome shotgun
sequence; 483624383; NZ_AMQU01000007.1
4085; Amphibacillus jilinensis Y1 Scaffold2, whole genome shotgun
sequence; 483992405; NZ_JH976435.1
4086; Nocardiopsis alba DSM 43377 contig_34, whole genome shotgun
sequence; 484007204; NZ_ANAC01000034.1
4087; Nocardiopsis halophila DSM 44494 contig_138, whole genome shotgun
sequence; 484007841; NZ_ANAD01000138.1
4088; Nocardiopsis halophila DSM 44494 contig_138, whole genome shotgun
sequence; 484007841; NZ_ANAD01000138.1
4089; Nocardiopsis halophila DSM 44494 contig_197, whole genome shotgun
sequence; 484008051; NZ_ANAD01000197.1
4090; Nocardiopsis baichengensis YIM 90130 Scaffold15_1, whole genome
shotgun sequence; 484012558; NZ_ANAS01000033.1
4091; Nocardiopsis halotolerans DSM 44410 contig_26, whole genome
shotgun sequence; 484015294; NZ_ANAX01000026.1
4092; Nocardiopsis salina YIM 90010 contig_204, whole genome shotgun
sequence; 484023808; NZ_ANBF01000204.1
4093; Nocardiopsis chromatogenes YIM 90109 contig_59, whole genome
shotgun sequence; 484026076; NZ_ANBH01000059.1
4094; Nocardiopsis chromatogenes YIM 90109 contig_93, whole genome
shotgun sequence; 484026206; NZ_ANBH01000093.1
4095; Porphyrobacter sp. AAP82 Contig35, whole genome shotgun sequence;
484033307; NZ_ANFX01000035.1
4096; Blastomonas sp. AAP53 Contig14, whole genome shotgun sequence;
484033631;NZ_ANFZ01000014.1
4097; Paenibacillus sp. PAMC 26794 5104_29, whole genome shotgun
sequence; 484070054; NZ_ANHX01000029.1
4098; Oscillatoria sp. PCC 10802 Osc10802DRAFT_Contig7.7, whole
genome shotgun sequence; 484104632; NZ_KB235948.1
4099; Clostridium botulinum CB11/1-1 CB_contig000105, whole genome
shotgun sequence; 484141779; NZ_AORM01000006.1
4100; Actinopolyspora halophila DSM 43834 ActhaDRAFT_contig1.1_C,
whole genome shotgun sequence; 484203522; NZ_AQUI01000002.1
4101; Streptomyces sp. FxanaC1 B074DRAFT_scaffold_1.2_C, whole
genome shotgun sequence; 484227180; NZ_AQWO01000002.1
4102; Smaragdicoccus niigatensis DSM44881 = NBRC 103563 strain DSM
44881 F600DRAFT_scaffold00011.11_C, whole genome shotgun sequence;
484234624; NZ_AQXZ01000009.1
4103; Verrucomicrobium sp. 3C A37ADRAFT_scaffold1.1, whole genome
shotgun sequence; 483219562; NZ_KB901875.1
4104; Verrucomicrobium sp. 3C A37ADRAFT_scaffold1.1, whole genome
shotgun sequence; 483219562; NZ_KB901875.1
4105; Ancylobacter sp. FA202 A3M1DRAFT_scaffold1.1, whole genome
shotgun sequence; 483720774; NZ_KB904818.1
4106; Filamentous cyanobacterium ESFC-1 A3MYDRAFT_scaffold1.1,
whole genome shotgun sequence; 483724571; NZ_KB904821.1
4107; Streptomyces sp. CcalMP-8W B053DRAFT_scaffold_17.18, whole
genome shotgun sequence; 483961830; NZ_KB890924.1
4108; Streptomyces sp. ScaeMP-e10 B061DRAFT_scaffold_0.1, whole
genome shotgun sequence; 483967534; NZ_KB891296.1
4109; Streptomyces sp. CNB091 D581DRAFT_scaffold00010.10, whole
genome shotgun sequence; 484070161; NZ_KB898999.1
4110; Streptomyces sp. TOR3209 Contig613, whole genome shotgun
sequence; 484867902; NZ_AGNH01000613.1
4111; Bacillus oceanisediminis 2691 contig2644, whole genome shotgun
sequence; 485048843; NZ_ALEG01000067.1
4112; Bacillus sp. REN51N contig 2, whole genome shotgun sequence;
748816024; NZ_JXAB01000002.1
4113; Calothrix sp. PCC 7103 Cal7103DRAFT_CPM.6, whole genome
shotgun sequence; 485067373; NZ_KB217478.1
4114; Pseudanabaena sp. PCC 6802 Pse6802_scaffold_5, whole genome
shotgun sequence; 485067426; NZ_KB235914.1
4115; Actinomadura atramentaria DSM 43919 strain SF2197
G339DRAFT_scaffold00002.2, whole genome shotgun sequence; 485090585;
NZ_KB907209.1
4116; Novispirillum itersonii subsp. itersonii ATCC 12639
G365DRAFT_scaffold00001.1, whole genome shotgun sequence; 485091510;
NZ_KB907337.1
4117; Novispirillum itersonii subsp. itersonii ATCC 12639
G365DRAFT_scaffold00001.1, whole genome shotgun sequence; 485091510;
NZ_KB907337.1
4118; Paenibacillus polymyxa ATCC 842 PPt02_scaffold1, whole genome
shotgun sequence; 485269841; NZ_GL905390.1
4119; Streptomyces sp. SolWspMP-sol2th B083DRAFT_scaffold_17.18_C,
whole genome shotgun sequence; 654969845; NZ_ARPF01000020.1
4120; Mesorhizobium huakuii 7653Rgenome; 657121522; CP006581.1
4121; Paenibacillus sp. HW567 B212DRAFT_scaffold1.1, whole genome
shotgun sequence; 486346141; NZ_KB910518.1
4122; Bacillus sp. 123MFChir2 H280DRAFT_scaffold00030.30, whole
genome shotgun sequence; 487368297; NZ_KB910953.1
4123; Streptomyces canus 299MFChir4.1 H293DRAFT_scaffold00032.32,
whole genome shotgun sequence; 487385965; NZ_KB911613.1
4124; Nocardiopsis potens DSM 45234 contig 25, whole genome shotgun
sequence; 484017897; NZ_ANBB01000025.1
4125; Kribbella catacumbae DSM 19601 A3ESDRAFT_scaffold_7.8_C,
whole genome shotgun sequence; 484207511; NZ_AQUZ01000008.1
4126; Paenibacillus riograndensis SBR5 Contig78, whole genome shotgun
sequence; 485470216; NZ_A
4127; Lamprocystis purpurea DSM 4197 A39ODRAFT_scaffold_0.1, whole
genome shotgun sequence; 483254584; NZ_KB902362.1
4128; Nonomuraea coxensis DSM 45129 A3G7DRAFT_scaffold_4.5, whole
genome shotgun sequence; 483454700; NZ_KB903974.1
4129; Spirosoma spitsbergense DSM 19989 B157DRAFT_scaffold_76.77,
whole genome shotgun sequence; 483994857; NZ_KB893599.1
4130; Amycolatopsis benzoatilytica AK 16/65 AmybeDRAFT_scaffold1.1,
whole genome shotgun sequence; 486399859; NZ_KB912942.1
4131; Amycolatopsis nigrescens CSC17Ta-90 AmyniDRAFT_Contig68.1_C,
whole genome shotgun sequence; 487404592; NZ_ARVW01000001.1
4132; Amycolatopsis nigrescens CSC17Ta-90 AmyniDRAFT_Contig68.1_C,
whole genome shotgun sequence; 487404592; NZ_ARVW01000001.1
4133; Reyranella massiliensis 521, whole genome shotgun sequence;
484038067; NZ_HE997181.1
4134; Acidobacteriaceae bacterium KBS 83 G002DRAFT_scaffold00007.7,
whole genome shotgun sequence; 485076323; NZ_KB906739.1
4135; Paenibacillus alvei A6-6i-x PAAL66ix_14, whole genome shotgun
sequence; 528200987; ATMS01000061.1
4136; Dehalobacter sp. UNSWDHB Contig_139, whole genome shotgun
sequence; 544905305; NZ_AUUR01000139.1
4137; Thermoactinomyces vulgaris strain NRRL F-5595 F5595contig15.1,
whole genome shotgun sequence; 929862756; NZ_LGKI01000090.1
4138; Clostridium saccharobutylicum DSM 13864, complete genome;
550916528; NC_022571.1
4139; Butyrivibrio fibrisolvens AB2020 G616DRAFT_scaffold00015.15_C,
whole genome shotgun sequence; 551012921; NZ_ATVZ01000015.1
4140; Butyrivibrio sp. XPD2006 G590DRAFT_scaffold00008.8_C, whole
genome shotgun sequence; 551021553; NZ_ATVT01000008.1
4141; Acidobacteriaceae bacterium TAA166 strain TAA 166
H979DRAFT_scaffold_0.1_C, whole genome shotgun sequence; 551216990;
NZ_ATWD01000001.1
4142; Acidobacteriaceae bacterium TAA166 strain TAA 166
H979DRAFT_scaffold_0.1_C, whole genome shotgun sequence; 551216990;
NZ_ATWD01000001.1
4143; Leptolyngbya sp. Heron Island J 50, whole genome shotgun sequence;
553739852; NZ_AWNH01000066.1
4144; Leptolyngbya sp. Heron Island J 50, whole genome shotgun sequence;
553739852; NZ_AWNH01000066.1
4145; Leptolyngbya sp. Heron Island J 67, whole genome shotgun sequence;
553740975; NZ_AWNH01000084.1
4146; Rothia aeria F0184 R_aeriaHMPREF0742-1.0_Cont136.4, whole
genome shotgun sequence; 551695014; AXZG01000035.1
4147; Gloeobacter kilaueensis JS1, complete genome; 554634310;
NC_022600.1
4148; Gloeobacter kilaueensis JS1, complete genome; 554634310;
NC_022600.1
4149; Asticcacaulis sp. AC466 contig00033, whole genome shotgun sequence;
557835508; NZ_AWGE01000033.1
4150; Streptomyces niveus NCIMB 11891 contig00003, whole genome
shotgun sequence; 558542923; AWQW01000003.1
4151; Streptomyces roseochromogenus subsp. oscitans DS 12.976
chromosome, whole genome shotgun sequence; 566155502;
NZ_CM002285.1
4152; Streptomyces roseochromogenus subsp. oscitans DS 12.976
chromosome, whole genome shotgun sequence; 566155502;
NZ_CM002285.1
4153; Bacillus boroniphilus JCM 21738 DNA, contig: contig_6, whole
genome shotgun sequence; 571146044; BAUW01000006.1
4154; Mesorhizobium sp. LSJC285A00 scaffold0007, whole genome shotgun
sequence; 563442031; NZ_AYVK01000007.1
4155; Mesorhizobium sp. LSJC277A00 scaffold0014, whole genome shotgun
sequence; 563459186; NZ_AYVM01000014.1
4156; Mesorhizobium sp. LNJC405B00 scaffold0005, whole genome shotgun
sequence; 563523441; NZ_AYWC01000005.1
4157; Mesorhizobium sp. LSJC265A00 scaffold0015, whole genome shotgun
sequence; 563472037; NZ_AYVP01000015.1
4158; Mesorhizobium sp. LSHC426A00 scaffold0005, whole genome shotgun
sequence; 563492715; NZ_AYVV01000005.1
4159; Mesorhizobium sp. LNHC232B00 scaffold0020, whole genome
shotgun sequence; 563561985; NZ_AYWP01000020.1
4160; Mesorhizobium sp. L48C026A00 scaffold0030, whole genome shotgun
sequence; 563848676; NZ_AYWU01000030.1
4161; Mesorhizobium sp. L2C089B000 scaffold0011, whole genome shotgun
sequence; 563888034; NZ_AYWV01000011.1
4162; Mesorhizobium sp. L2C084A000 scaffold0007, whole genome shotgun
sequence; 563938926; NZ_AYWX01000007.1
4163; Clostridium pasteurianum NRRL B-598, complete genome; 930593557;
NZ_CP011966.1
4164; Paenibacillus polymyxa CR1, complete genome; 734699963;
NC_023037.2
4165; Clostridium butyricum DORA_1 Q607_CBUC00058, whole genome
shotgun sequence; 566226100; AZLX01000058.1
4166; Streptococcus suis strain LS8F, whole genome shotgun sequence;
766589647; NZ_CEHJ01000007.1
4167; Mycobacterium sp. UM_Kg27 contig000002, whole genome shotgun
sequence; 809025315; NZ_JRMM01000002.1
4168; Mycobacterium iranicum UM_TJL Contig_42, whole genome shotgun
sequence; 638987534; NZ_AUWT01000042.1
4169; Paenibacillus sp. MAEPY2 contig7, whole genome shotgun sequence;
639451286;NZ_AWUK01000007.1
4170; Verrucomicrobia bacterium LP2A
G346DRAFT_scf7180000000012_quiver.2_C, whole genome shotgun
sequence; 640169055; NZ_JAFS01000002.1
4171; Verrucomicrobia bacterium LP2A
G346DRAFT_scf7180000000012_quiver.2_C, whole genome shotgun
sequence; 640169055; NZ_JAFS01000002.1
4172; Bacillus mannanilyticus JCM 10596, whole genome shotgun sequence;
640600411; NZ_BAMO01000071.1
4173; Bifidobacterium breve NCFB 2258, complete genome; 749295448;
NZ_CP006714.1
4174; Haloglycomyces albus DSM 45210 HalalDRAFT_chromosome1.1_C,
whole genome shotgun sequence; 644043488; NZ_AZUQ01000001.1
4175; Kutzneria albida strain NRRL B-24060 contig305.1, whole genome
shotgun sequence; 662161093; NZ_JNYH01000515.1
4176; Kutzneria albida strain NRRL B-24060 contig305.1, whole genome
shotgun sequence; 662161093; NZ_JNYH01000515.1
4177; Kutzneria albida DSM 43870, complete genome; 754862786;
NZ_CP007155.1
4178; Paenibacillus sp. 1-49 S149_contig00281, whole genome shotgun
sequence; 647230448; NZ_ASRY01000102.1
4179; Paenibacillus graminis RSA19 S2_contig00597, whole genome shotgun
sequence; 647256651; NZ_ASSG01000304.1
4180; Paenibacillus sp. 1-18 S118_contig00103, whole genome shotgun
sequence; 647269417; NZ_ASSB01000031.1
4181; Paenibacillus polymyxa TD94 STD94_contig00759, whole genome
shotgun sequence; 647274605; NZ_ASSA01000134.1
4182; Bacillus flexus T6186-2 contig_106, whole genome shotgun sequence;
647636934; NZ_JANV01000106.1
4183; Mastigocladopsis repens PCC 10914 Mas10914DRAFT_scaffold1.1,
whole genome shotgun sequence; 482909462; NZ_JH992901.1
4184; Streptomyces sp. FxanaC1 B074DRAFT_scaffold_7.8_C, whole
genome shotgun sequence; 484227195; NZ_AQWO01000008.1
4185; Streptomyces sp. HmicA12 B072DRAFT_scaffold_19.20, whole
genome shotgun sequence; 483972948; NZ_KB891808.1
4186; Butyrivibrio sp. XPD2002 G587DRAFT_scaffold00011.11, whole
genome shotgun sequence; 651381584; NZ_KE384117.1
4187; Butyrivibrio sp. NC3005 G634DRAFT_scaffold00001.1, whole
genome shotgun sequence; 651394394; NZ_KE384206.1
4188; Paenarthrobacter nicotinovorans 231Sha2.1M6
I960DRAFT_scaffold00004.4_C, whole genome shotgun sequence;
651445346; NZ_AZVC01000006.1
4189; Bacillus sp. J37 BacJ37DRAFT_scaffold_0.1_C, whole genome
shotgun sequence; 651516582; NZ_JAEK01000001.1
4190; Bacillus sp. UNC437CL72CviS29 M014DRAFT_scaffold00009.9_C,
whole genome shotgun sequence; 651596980; NZ_AXVB01000011.1
4191; Bacillus bogoriensis ATCC BAA-922
T323DRAFT_scaffold00008.8_C, whole genome shotgun sequence;
651937013; NZ_JHYI01000013.1
4192; Bacillus kribbensis DSM 17871 H539DRAFT_scaffold00003.3, whole
genome shotgun sequence; 651983111; NZ_KE387239.1
4193; Fischerella sp. PCC 9431 Fis9431DRAFT_Scaffold1.2, whole genome
shotgun sequence; 652326780; NZ_KE650771.1
4194; Fischerella sp. PCC 9605 FIS9605DRAFT_scaffold2.2, whole genome
shotgun sequence; 652337551; NZ_KI912149.1
4195; Clostridium akagii DSM 12554 BR66DRAFT_scaffold00010.10_C,
whole genome shotgun sequence; 652488076; NZ_JMLK01000014.1
4196; Clostridium beijerinckii HUN142 T483DRAFT_scaffold00004.4, whole
genome shotgun sequence; 652494892; NZ_KK211337.1
4197; Mesorhizobium sp. URHA0056 H959DRAFT_scaffold00004.4_C,
whole genome shotgun sequence; 652670206; NZ_AUEL01000005.1
4198; Mesorhizobium loti R88b Meslo2DRAFT_Scaffold1.1, whole genome
shotgun sequence; 652688269; NZ_KI912159.1
4199; Mesorhizobium ciceri WSM4083 MESCI2DRAFT_scaffold_0.1,
whole genome shotgun sequence; 652698054; NZ_KI912610.1
4200; Mesorhizobium sp. URHC0008 N549DRAFT_scaffold00001.1_C,
whole genome shotgun sequence; 652699616; NZ_JIAP01000001.1
4201; Mesorhizobium erdmanii USDA 3471 A3AUDRAFT_scaffold_7.8_C,
whole genome shotgun sequence; 652719874; NZ_AXAE01000013.1
4202; Mesorhizobium loti CJ3sym A3A9DRAFT_scaffold_25.26_C, whole
genome shotgun sequence; 652734503; NZ_AXAL01000027.1
4203; Cohnella thermotolerans DSM 17683
G485DRAFT_scaffold00041.41_C, whole genome shotgun sequence;
652787974; NZ_AUCP01000055.1
4204; Mesorhizobium sp. WSM3626 Mesw3626DRAFT_scaffold_6.7_C,
whole genome shotgun sequence; 652879634; NZ_AZUY01000007.1
4205; Mesorhizobium sp. WSM1293 MesloDRAFT_scaffold_4.5, whole
genome shotgun sequence; 652910347; NZ_KI911320.1
4206; Mesorhizobium sp. WSM3224 YU3DRAFT_scaffold_3.4_C, whole
genome shotgun sequence; 652912253; NZ_AIY001000004.1
4207; Butyrivibrio fibrisolvens MD2001 G635DRAFT_scaffold00033.33_C,
whole genome shotgun sequence; 652963937; NZ_AUKD01000034.1
4208; Legionella pneumophila subsp. pneumophila strain ATCC 33155
contig032, whole genome shotgun sequence; 652971687;
NZ_JFIN01000032.1
4209; Legionella pneumophila subsp. pneumophila strain ATCC 33154
Scaffold2, whole genome shotgun sequence; 653016013; NZ_KK074241.1
4210; Legionella pneumophila subsp. pneumophila strain ATCC 33823
Scaffold7. whole genome shotgun sequence; 653016661; NZ_KK074199.1
4211; Bacillus sp. URHB0009 H980DRAFT_scaffold00016.16_C, whole
genome shotgun sequence; 653070042; NZ_AUER01000022.1
4212; Lachnospira multipara ATCC 19207 G600DRAFT_scaffold00009.9_C,
whole genome shotgun sequence; 653218978; NZ_AUJG01000009.1
4213; Streptomyces sp. CNH099 B121DRAFT_scaffold_16.17_C, whole
genome shotgun sequence; 654239557; NZ_AZWL01000018.1
4214; Desulfatiglans anilini DSM 4660 H567DRAFT_scaffold00005.5_C,
whole genome shotgun sequence; 654868823; NZ_AULM01000005.1
4215; Legionella pneumophila subsp. fraseri strain ATCC 35251 contig031,
whole genome shotgun sequence; 654928151; NZ_JFIG01000031.1
4216; Bacillus sp. FJAT-14578 Scaffold2, whole genome shotgun sequence;
654948246; NZ_KI632505.1
4217; Bacillus sp. 278922_107 H622DRAFT_scaffold00001.1, whole genome
shotgun sequence; 654964612; NZ_KI911354.1
4218; Ruminococcus flavefaciens ATCC 19208
L870DRAFT_scaffold00001.1, whole genome shotgun sequence; 655069822;
NZ_KI912489.1
4219; Paenibacillus taiwanensis DSM 18679
H509DRAFT_scaffold00010.10_C, whole genome shotgun sequence;
655095554; NZ_AULE01000001.1
4220; Paenibacillus sp. UNC451MF BP97DRAFT_scaffold0008.18_C,
whole genome shotgun sequence; 655103160; NZ_JMLS01000021.1
4221; Paenibacillus pinihumi DSM 23905 = JCM 16419 strain DSM 23905
H583DRAFT_scaffold00005.5, whole genome shotgun sequence; 655115689;
NZ_KE383867.1
4222; Paenibacillus harenae DSM 16969 H581DRAFT_scaffold00002.2,
whole genome shotgun sequence; 655165706; NZ_KE383843.1
4223; Shimazuella kribbensis DSM 45090 A3GQDRAFT_scaffold_0.1_C,
whole genome shotgun sequence; 655370026; NZ_ATZF01000001.1
4224; Shimazuella kribbensis DSM 45090 A3GQDRAFT_scaffold_5.6_C,
whole genome shotgun sequence; 655371438; NZ_ATZF01000006.1
4225; Streptomyces flavidovirens DSM 40150
G412DRAFT_scaffold00007.7_C, whole genome shotgun sequence;
655414006; NZ_AUBE01000007.1
4226; Streptomyces flavidovirens DSM 40150
G412DRAFT_scaffold00009.9, whole genome shotgun sequence; 655416831;
NZ_KE386846.1
4227; Azospirillum halopraeferens DSM 3675
G472DRAFT_scaffold00039.39_C, whole genome shotgun sequence;
655967838; NZ_AUCF01000044.1
4228; Clostridium scatologenes strain ATCC 25775, complete genome;
802929558; NZ_CP009933.1
4229; Paenibacillus harenae DSM 16969 H581DRAFT_scaffold00004.4,
whole genome shotgun sequence; 656245934; NZ_KE383845.1
4230; Paenibacillus alginolyticus DSM 5050 = NBRC 15375 strain DSM 5050
G519DRAFT_scaffold00043.43_C, whole genome shotgun sequence;
656249802; NZ_AUGY01000047.1
4231; Paenibacillus alginolyticus DSM 5050 = NBRC 15375 strain DSM 5050
G519DRAFT_scaffold00043.43_C, whole genome shotgun sequence;
656249802; NZ_AUGY01000047.1
4232; Bacillus indicus strain DSM 16189 Contig01, whole genome shotgun
sequence; 737222016; NZ_JNVC02000001.1
4233; Bacillus sp. RP1137 contig_18, whole genome shotgun sequence;
657210762; NZ_AXZS01000018.1
4234; Streptomyces leeuwenhoekii strain C34(2013) c34_sequence_0012,
whole genome shotgun sequence; 657294764; NZ_AZSD01000012.1
4235; Streptomyces leeuwenhoekii strain C34(2013) c34_sequence_0041,
whole genome shotgun sequence; 657295264; NZ_AZSD01000040.1
4236; Streptomyces leeuwenhoekii strain C58 contig69, whole genome
shotgun sequence; 873282617; NZ_LFEH01000068.1
4237; Bacillus thuringiensis LM1212 scaffold_08, whole genome shotgun
sequence; 657629081; NZ_AYPV01000024.1
4238; Paenibacillus polymyxa strain WLY78 S6_contig00095, whole genome
shotgun sequence; 657719467; NZ_ALJV01000094.1
4239; [Scytonema hofmanni] UTEX 2349 Tol9009DRAFT_TPD.8, whole
genome shotgun sequence; 657935980; NZ_KK073768.1
4240; Sphingomonas sp. DC-6 scaffold87, whole genome shotgun sequence;
662140302; NZ_JMUB01000087.1
4241; Streptomyces lavendulae strain Fujisawa #8006 contig417.1, whole
genome shotgun sequence; 662043624; NZ_JNXL01000469.1
4242; Streptomyces sp. NRRL WC-3773 contig36.1, whole genome shotgun
sequence; 664487325; NZ_JOJI01000036.1
4243; Streptomyces flavotricini strain NRRL B-5419 contig237.1, whole
genome shotgun sequence; 662063073; NZ_JNXV01000303.1
4244; Streptomyces peruviensis strain NRRL ISP-5592
P181_Doro1_scaffold152, whole genome shotgun sequence; 662097244;
NZ_KL575165.1
4245; Streptomyces natalensis ATCC 27448 Scaffold_33, whole genome
shotgun sequence; 764439507; NZ_JRKI01000027.1
4246; Streptomyces decoyicus strain NRRL ISP-5087
P056_Doro1_scaffold78, whole genome shotgun sequence; 662133033;
NZ_KL570321.1
4247; Streptomyces baarnensis strain NRRL B-2842 P144_Doro1_scaffold26,
whole genome shotgun sequence; 662135579; NZ_KL573564.1
4248; Streptomyces vinaceus strain NRRL ISP-5257 contig5.1, whole genome
shotgun sequence; 759527818; NZ_JNYP01000005.1
4249; Spirillospora albida strain NRRL B-3350 contig1.1, whole genome
shotgun sequence; 663122276; NZ_JOFJ01000001.1
4250; Streptomyces sp. NRRL S-455 contig1.1, whole genome shotgun
sequence; 663192162; NZ_JOCT01000001.1
4251; Streptomyces sp. NRRL S-87 contig69.1, whole genome shotgun
sequence; 663169513; NZ_JO
4252; Streptomyces katrae strain NRRL B-16271 contig33.1, whole genome
shotgun sequence; 663300513; NZ_JNZY01000033.1
4253; Streptomyces katrae strain NRRL B-16271 contig37.1, whole genome
shotgun sequence; 663300941; NZ_JNZY01000037.1
4254; Streptomyces sp. NRRL B-3229 contig5.1, whole genome shotgun
sequence; 663316931; NZ_JOGP01000005.1
4255; Streptomyces ruber strain NRRL B-1661 contig94.1, whole genome
shotgun sequence; 663365281; NZ_JODN01000094.1
4256; Streptomyces roseoverticillatus strain NRRL B-3500 contig22.1, whole
genome shotgun sequence; 663372343; NZ_JOFL01000022.1
4257; Streptomyces roseoverticillatus strain NRRL B-3500 contig43.1, whole
genome shotgun sequence; 663373497; NZ_JOFL01000043.1
4258; Streptomyces rimosus subsp. rimosus strain NRRL WC-3869
P248contig20.1, whole genome shotgun sequence; 925322461;
LGCQ01000113.1
4259; Streptomyces sp. NRRL B-12105 contig1.1, whole genome shotgun
sequence; 663380895; NZ_JNZW01000001.1
4260; Streptomyces sp. NRRL S-1448 contig 134.1, whole genome shotgun
sequence; 663421576; NZ_JOGE01000134.1
4261; Allokutzneria albata strain NRRL B-24461 contig22.1, whole genome
shotgun sequence; 663596322; NZ_JOEF01000022.1
4262; Herbidospora cretacea strain NRRL B-16917 contig7.1, whole genome
shotgun sequence; 663670981; NZ_JODQ01000007.1
4263; Nocardia sp. NRRL WC-3656 contig2.1, whole genome shotgun
sequence; 663737675; NZ_JOJF01000002.1
4264; Streptomyces aureocirculatus strain NRRL ISP-5386 contig11.1, whole
genome shotgun sequence; 664013282; NZ_JOAP01000011.1
4265; Streptomyces cyaneofuscatus strain NRRL B-2570 contig9.1, whole
genome shotgun sequence; 664021017; NZ_JOEM01000009.1
4266; Streptomyces aureocirculatus strain NRRL ISP-5386 contig49.1, whole
genome shotgun sequence; 664026629; NZ_JOAP01000049.1
4267; Streptomyces sclerotialus strain NRRL B-2317 contig7.1, whole genome
shotgun sequence; 664034500; NZ_JODX01000007.1
4268; Streptomyces anulatus strain NRRL B-2873 contig21.1, whole genome
shotgun sequence; 664049400; NZ_JOEZ01000021.1
4269; Streptomyces globisporus subsp. globisporus strain NRRL B-2709
contig24.1, whole genome shotgun sequence; 664051798;
NZ_JNZK01000024.1
4270; Streptomyces rimosus subsp. rimosus strain NRRL B-2660 contig14.1,
whole genome shotgun sequence; 664052786; NZ_JOES01000014.1
4271; Streptomyces achromogenes subsp. achromogenes strain NRRL B-2120
contig2.1, whole genome shotgun sequence; 664063830;
NZ_JODT01000002.1
4272; Streptomyces rimosus subsp. rimosus strain NRRL B-2660 contig124.1,
whole genome shotgun sequence; 664066234; NZ_JOES01000124.1
4273; Streptomyces rimosus subsp. rimosus strain NRRL WC-3927 contig5.1,
whole genome shotgun sequence; 664091759; NZ_JOBO01000005.1
4274; Streptomyces rimosus subsp. rimosus strain NRRL WC-3904
contig10.1, whole genome shotgun sequence; 664126885;
NZ_JOCQ01000010.1
4275; Streptomyces rimosus subsp. rimosus strain NRRL WC-3904
contig106.1, whole genome shotgun sequence; 664141810;
NZ_JOCQ01000106.1
4276; Streptomyces sp. NRRL F-2295 P395contig79.1, whole genome
shotgun sequence; 926288193; NZ_LGCY01000146.1
4277; Streptomyces lavenduligriseus strain NRRL ISP-5487 contig2.1, whole
genome shotgun sequence; 664244706; NZ_JOBD01000002.1
4278; Streptomyces lavenduligriseus strain NRRL ISP-5487 contig2.1, whole
genome shotgun sequence; 664244706; NZ_JOBD01000002.1
4279; Streptomyces alboniger strain NRRL B-1832 B-1832_contig_384,
whole genome shotgun sequence; 943899498; NZ_LIQN01000384.1
4280; Streptomyces sp. NRRL S-337 contig31.1, whole genome shotgun
sequence; 664275807; NZ_JOIX01000031.1
4281; Streptomyces sp. NRRL S-337 contig41.1, whole genome shotgun
sequence; 664277815; NZ_JOIX01000041.1
4282; Streptomyces hygroscopicus subsp. hygroscopicus strain NRRL B-1477
contig8.1, whole genome shotgun sequence; 664299296;
NZ_JOIK01000008.1
4283; Streptomyces sp. NRRL F-4474 contig32.1, whole genome shotgun
sequence; 664323078; NZ_JOIB01000032.1
4284; Streptomyces sp. NRRL S-475 contig32.1, whole genome shotgun
sequence; 664325162; NZ_JOJB01000032.1
4285; Streptomyces sp. NRRL S-1868 contig54.1, whole genome shotgun
sequence; 664360925; NZ_JOGD01000054.1
4286; Streptomyces sp. NRRL S-646 contig23.1, whole genome shotgun
sequence; 664421883; NZ_JODC01000023.1
4287; Streptomyces sp. NRRL S-1813 contig13.1, whole genome shotgun
sequence; 664466568; NZ_JOHB01000013.1
4288; Streptomyces sp. NRRL WC-3773 contig2.1, whole genome shotgun
sequence; 664478668; NZ_JOJI01000002.1
4289; Streptomyces sp. NRRL WC-3773 contig11.1, whole genome shotgun
sequence; 664481891; NZ_JOJI01000011.1
4290; Streptomyces sp. NRRL WC-3773 contig36.1, whole genome shotgun
sequence; 664487325; NZ_JOJI01000036.1
4291; Streptomyces olivaceus strain NRRL B-3009 contig20.1, whole genome
shotgun sequence; 664523889; NZ_JOFH01000020.1
4292; Streptomyces sp. NRRL F-5702 contig3.1, whole genome shotgun
sequence; 664537198; NZ_JOHD01000003.1
4293; Streptomyces ochraceiscleroticus strain NRRL ISP-5594 contig9.1,
whole genome shotgun sequence; 664540649; NZ_JOAX01000009.1
4294; Streptomyces sp. NRRL S-118 P205_Doro1_scaffold2, whole genome
shotgun sequence; 664556736; NZ_KL591003.1
4295; Streptomyces sp. NRRL WC-3641 P206_Doro1_scaffold18, whole
genome shotgun sequence; 664607641; NZ_KL579016.1
4296; Streptomyces sp. NRRL S-623 contig14.1, whole genome shotgun
sequence; 665522165; NZ_JOJC01000016.1
4297; Streptomyces sp. NRRL WC-3719 contig152.1, whole genome shotgun
sequence; 665536304; NZ_JOCD01000152.1
4298; Streptomyces duihamensis strain NRRL B-3309 contig3.1, whole
genome shotgun sequence; 665586974; NZ_JNXR01000003.1
4299; Streptomyces duihamensis strain NRRL B-3309 contig23.1, whole
genome shotgun sequence; 665604093; NZ_JNXR01000023.1
4300; Bacillus sp. MB2021 T349DRAFT_scaffold00010.10_C, whole
genome shotgun sequence; 671553628; NZ_JNJJ01000011.1
4301; Lachnospira multipara LB2003 T537DRAFT_scaffold00010.10_C,
whole genome shotgun sequence; 671578517; NZ_JNKW01000011.1
4302; Clostridium drakei strain SL1 contig_20, whole genome shotgun
sequence; 692121046; NZ_JIBU02000020.1
4303; Rhodococcus fascians A21d2 contig10, whole genome shotgun
sequence; 739287390; NZ_JMFA01000010.1
4304; Streptomyces alboviridis strain NRRL B-1579 contig18.1, whole
genome shotgun sequence; 695845602; NZ_JNWU01000018.1
4305; Streptomyces sp. JS01 contig2, whole genome shotgun sequence;
695871554; NZ_JPWW01000002.1
4306; Streptomyces albus subsp. albus strain NRRL B-16041 contig28.1,
whole genome shotgun sequence; 695870063; NZ_JNWW01000028.1
4307; Streptomyces rimosus subsp. rimosus strain NRRL B-16073 contig7.1,
whole genome shotgun sequence; 696493030; NZ_JNWX01000007.1
4308; Streptomyces peucetius strain NRRL WC-3868 contig49.1, whole
genome shotgun sequence; 665671804; NZ_JOCK01000052.1
4309; Blautia producta strain ER3 contig 8, whole genome shotgun sequence;
696661199; NZ_JPJF01000008.1
4310; Streptomyces albus subsp. albus strain NRRL B-1811 contig32.1, whole
genome shotgun sequence; 665618015; NZ_JODR01000032.1
4311; Streptomyces lydicus strain NRRL ISP-5461 contig41.1, whole genome
shotgun sequence; 702808005; NZ_JNZA01000041.1
4312; Streptomyces iakyrus strain NRRL ISP-5482 contig6.1, whole genome
shotgun sequence; 702914619; NZ_JNXI01000006.1
4313; Kibdelosporangium aridum subsp. largum strain NRRL B-24462
contig91.4, whole genome shotgun sequence; 703243970;
NZ_JNYM01001429.1
4314; Streptomyces galbus strain KCCM 41354 contig00021, whole genome
shotgun sequence; 716912366; NZ_JRHJ01000016.1
4315; Bacillus aryabhattai strain GZ03 contig1_scaffold1, whole genome
shotgun sequence; 723602665; NZ_JPIE01000001.1
4316; Bacillus cereus R309803 chromosome, whole genome shotgun
sequence; 238801472; NZ_CM000720.1
4317; Bacillus cereus AH603 chromosome, whole genome shotgun sequence;
238801489; NZ_CM000737.1
4318; Sphingomonas sp. 37zxx contig3_scaffold2, whole genome shotgun
sequence; 728813405; NZ_JROH01000003.1
4319; Lachnospira multipara MC2003 T520DRAFT_scaffold00007.7_C,
whole genome shotgun sequence; 653225243; NZ_JHWY01000011.1
4320; Bacillus sp. 72 T409DRAFT_scf7180000000077_quiver.15_C, whole
genome shotgun sequence; 736160933; NZ_JQMI01000015.1
4321; Bacillus simplex BA2H3 scaffold2, whole genome shotgun sequence;
736214556; NZ_KN360955.1
4322; Bacillus manliponensis strain JCM 15802 contig4, whole genome
shotgun sequence; 736629899; NZ_JOTN01000004.1
4323; Bacillus vietnamensis strain HD-02, whole genome shotgun sequence;
736762362; NZ_CCDN010000009.1
4324; Erythrobacter longus strain DSM 6997 contig9, whole genome shotgun
sequence; 736965849; NZ_JMIW01000009.1
4325; Calothrix sp. 336/3, complete genome; 821032128; NZ_CP011382.1
4326; Desulfobacter vibrioformis DSM 8776
Q366DRAFT_scaffold00036.35_C, whole genome shotgun sequence;
737257311; NZ_JQKJ01000036.1
4327; Actinokineospora spheciospongiae strain EG49 contig1268_1, whole
genome shotgun sequence; 737301464; NZ_AYXG01000139.1
4328; Bacillus firmus DS1 scaffold33, whole genome shotgun sequence;
737350949; NZ_APVL01000034.1
4329; Bacillus hemicellulosilyticus JCM 9152, whole genome shotgun
sequence; 737360192; NZ_BAUU01000008.1
4330; Edaphobacter aggregans DSM 19364
Q363DRAFT_scaffold00032.32_C, whole genome shotgun sequence;
737370143; NZ_JQKI01000040.1
4331; Halobacillus sp. BBL2006 cont444, whole genome shotgun sequence;
737576092; NZ_JRNX01000441.1
4332; Bacillus akibai JCM 9157, whole genome shotgun sequence;
737696658; NZ_BAUV01000025.1
4333; Frankia sp. CeD CEDDRAFT_scaffold_22.23, whole genome shotgun
sequence; 737947180; NZ_JPGU01000023.1
4334; Fusobacterium necrophorum BFIR-2 contig0075, whole genome
shotgun sequence; 737951550; NZ_JAAG01000075.1
4335; [Leptolyngbya] sp. JSC-1
Osccy1DRAFT_CYJSC1_DRAF_scaffold00069.1, whole genome shotgun
sequence; 738050739; NZ_KL662191.1
4336; Lysobacter daejeonensis GH1-9 contig23, whole genome shotgun
sequence; 738180952; NZ_AVPU01000014.1
4337; Mastigocoleus testarum BC008 Contig-2, whole genome shotgun
sequence; 959926096; NZ_LMTZ01000085.1
4338; Myxosarcina sp. GI1 contig_5, whole genome shotgun sequence;
738529722; NZ_JRFE01000006.1
4339; Paenibacillus sp. FSL H7-689 Contig015, whole genome shotgun
sequence; 738716739; NZ_ASPU01000015.1
4340; Paenibacillus sp. FSL R7-269 Contig022, whole genome shotgun
sequence; 738803633; NZ_ASPS01000022.1
4341; Paenibacillus sp. FSL R7-277 Contig088, whole genome shotgun
sequence; 738841140; NZ_ASPX01000088.1
4342; Prevotella oryzae DSM 17970 XylorDRAFT_XOA.1, whole genome
shotgun sequence; 738999090; NZ_KK073873.1
4343; Rothia dentocariosa strain C6B contig_5, whole genome shotgun
sequence; 739372122; NZ_JQHE01000003.1
4344; Ruminococcus albus 8 contig00035, whole genome shotgun sequence;
325680876; NZ_ADKM02000123.1
4345; Amycolatopsis orientalis DSM 40040 = KCTC 9412 contig 32, whole
genome shotgun sequence; 499136900; NZ_ASJB01000015.1
4346; Streptococcus salivarius strain NU10 contig_11, whole genome shotgun
sequence; 739748927; NZ_JJMT01000011.1
4347; Streptomyces griseorubens strain JSD-1 contig143, whole genome
shotgun sequence; 657284919; JJMG01000143.1
4348; Streptomyces avermitilis MA-4680 = NBRC 14893, complete genome;
162960844; NC_003155.4
4349; Streptomyces avermitilis MA-4680 = NBRC 14893, complete genome;
162960844; NC_003155.4
4350; Streptomyces aurantiacus JA 4570 Seq28, whole genome shotgun
sequence; 514916412; NZ_AOPZ01000028.1
4351; Streptomyces griseus subsp. griseus strain NRRL WC-3645 contig39.1,
whole genome shotgun sequence; 739830131; NZ_JOJE01000039.1
4352; Streptomyces griseus subsp. griseus strain NRRL WC-3645 contig40.1,
whole genome shotgun sequence; 739830264; NZ_JOJE01000040.1
4353; Streptomyces scabiei strain NCPPB 4086 scf_65433_365.1, whole
genome shotgun sequence; 739854483; NZ_KL997447.1
4354; Streptomyces sp. FXJ7.023 Contig10, whole genome shotgun sequence;
510871397; NZ_APIV01000010.1
4355; Streptomyces sp. NRRL F-5053 contig1.1, whole genome shotgun
sequence; 664356765; NZ_JOHT01000001.1
4356; Streptomyces viridochromogenes Tue57 Seq127, whole genome
shotgun sequence; 443625867; NZ_AMLP01000127.1
4357; Streptomyces sp. Tu 6176 scaffold00003, whole genome shotgun
sequence; 740044478; NZ_KK106990.1
4358; Streptomyces toyocaensis strain NRRL 15009 contig00064, whole
genome shotgun sequence; 740092143; NZ_JFCB01000064.1
4359; Streptomyces sp. PRh5 contig001, whole genome shotgun sequence;
740097110; NZ_JABQ01000001.1
4360; Tolypothrix bouteillei VB521301 scaffold_1, whole genome shotgun
sequence; 910242069; NZ_JHEG02000048.1
4361; Thioclava indica strain DT23-4 contig29, whole genome shotgun
sequence; 740292158; NZ_AUNB01000028.1
4362; Streptomyces albulus strain NK660, complete genome; 754221033;
NZ_CP007574J
4363; Paenibacillus sp. FSLH7-0357, complete genome; 749299172;
NZ_CP009241.1
4364; Paenibacillus stellifer strain DSM 14472, complete genome; 753871514;
NZ_CP009286.1
4365; Brevundimonas nasdae strain TPW30 Contig_13, whole genome
shotgun sequence; 746187665; NZ_JWSY01000013.1
4366; Paenibacillus polymyxa strain DSM 365 Contig001, whole genome
shotgun sequence; 746220937; NZ_JMIQ01000001.1
4367; Paenibacillus sp. IHB B 3415 contig_069, whole genome shotgun
sequence; 746258261; NZ_JUEI01000069.1
4368; Streptomyces sp. 769, complete genome; 749181963; NZ_CP003987.1
4369; Hassallia byssoidea VB512170 scaffold_0, whole genome shotgun
sequence; 748181452; NZ_JTCM01000043.1
4370; Hassallia byssoidea VB512170 scaffold_0, whole genome shotgun
sequence; 748181452; NZ_JTCM01000043.1
4371; Jeotgalibacillus malaysiensis strain D5 chromosome, complete genome;
749182744; NZ_CP009416.1
4372; Paenibacillus sp. FSL R7-0273, complete genome; 749302091;
NZ_CP009283.1
4373; Paenibacillus jamilae strain NS115 contig_27, whole genome shotgun
sequence; 970428876; NZ_LDRX01000027.1
4374; Streptomonospora alba strain YIM 90003 contig_9, whole genome
shotgun sequence; 749673329; NZ_JROO01000009.1
4375; Actinobaculum sp. oral taxon 183 str. F0552 A_P1HMPREF0043-
1.0_Cont1.1, whole genome shotgun sequence; 541476958;
AWSB01000006.1
4376; Actinobaculum sp. oral taxon 183 str. F0552 Scaffold15, whole genome
shotgun sequence; 545327527; NZ_KE951412.1
4377; Actinobaculum sp. oral taxon 183 str. F0552 A_P1HMPREF0043-
1.0_Cont15.2, whole genome shotgun sequence; 541473965;
AWSB01000041.1
4378; Nocardia transvalensis NBRC 15921, whole genome shotgun sequence;
485125031; NZ_BAGL01000055.1
4379; Xenococcus sp. PCC 7305 scaffold00124, whole genome shotgun
sequence; 443325429; NZ_ALVZ01000124.1
4380; Mesorhizobium sp. ORS3324, whole genome shotgun sequence;
751265275; NZ_CCMY01000220.1
4381; Mesorhizobium plurifarium, whole genome shotgun sequence;
751292755; NZ_CCNE01000004.1
4382; Mesorhizobium sp. SOD10, whole genome shotgun sequence;
751285871; NZ_CCNA01000001.1
4383; Tolypothrix campylonemoides VB511288 scalfold_0, whole genome
shotgun sequence; 751565075; NZ_JXCB01000004.1
4384; Jeotgalibacillus campisalis strain SF-57 contig00001, whole genome
shotgun sequence; 751586078; NZ_JXRR01000001.1
4385; Jeotgalibacillus soli strain P9 contig00009, whole genome shotgun
sequence; 751619763; NZ_JXRP01000009.1
4386; Cylindrospermum stagnate PCC 7417, complete genome; 434402184;
NC_019757.1
4387; Bacillus sp. 1NLA3E, complete genome; 488570484; NC_021171.1
4388; Tistrella mobilis KA081020-065, complete genome; 389875858;
NC_017956.1
4389; Stackebrandtia nassauensis DSM 44728, complete genome; 291297538;
NC_013947.1
4390; Magnetospirillum gryphiswaldense MSR-1, WORKING DRAFT
SEQUENCE, 373 unordered pieces; 144897097; CU459003.1
4391; Clostridium beijerinckii strain NCIMB 14988 genome; 754484184;
NZ_CP010086.1
4392; Frankia alni str. ACN14A chromosome, complete sequence;
111219505; NC_008278.1
4393; Streptomyces sp. NBRC 110027, whole genome shotgun sequence;
754788309; NZ_BBNO01000002.1
4394; Streptomyces sp. NBRC 110027, whole genome shotgun sequence;
754796661; NZ_BBNO01000008.1
4395; Paenibacillus sp. FSL R7-0331, complete genome; 754821094;
NZ_CP009284.1
4396; Kibdelosporangium sp. MJ126-NF4, whole genome shotgun sequence;
754819815; NZ_CDME01000002.1
4397; Paenibacillus camerounensis strain G4, whole genome shotgun
sequence; 754841195; NZ_CCDG010000069.1
4398; Paenibacillus borealis strain DSM 13188, complete genome;
754859657; NZ_CP009285.1
4399; Streptacidiphilus neutrinimicus strain NBRC 100921, whole genome
shotgun sequence; 755016073; NZ_BBPO01000030.1
4400; Streptacidiphilus melanogenes strain NBRC 103184, whole genome
shotgun sequence; 755032408; NZ_BBPP01000024.1
4401; Streptacidiphilus anmyonensis strain NBRC 103185, whole genome
shotgun sequence; 755077919; NZ_BBPQ01000048.1
4402; Streptacidiphilus jiangxiensis strain NBRC 100920, whole genome
shotgun sequence; 755108320; NZ_BBPN01000056.1
4403; Mesorhizobium sp. ORS3359, whole genome shotgun sequence;
756828038; NZ_CCNC01000143.1
4404; Aneurinibacillus migulanus strain Nagano E1 contig_36, whole genome
shotgun sequence; 928874573; NZ_LIXL01000208.1
4405; Bifidobacterium reuteri DSM 23975 Contig04, whole genome shotgun
sequence; 672991374; JGZK01000004.1
4406; Streptomyces luteus strain TRM 45540 Scaffold1, whole genome
shotgun sequence; 759659849; NZ_KN039946.1
4407; Streptomyces nodosus strain ATCC 14899 genome; 759739811;
NZ_CP009313.1
4408; Streptomyces fradiae strain ATCC 19609 contig0008, whole genome
shotgun sequence; 759752221; NZ_JNAD01000008.1
4409; Streptomyces glaucescens strain GLA.O, complete genome; 759802587;
NZ_CP009438.1
4410; Nonomuraea Candida strain NRRL B-24552 contig8.1, whole genome
shotgun sequence; 759934284; NZ_JOAG01000009.1
4411; Nonomuraea Candida strain NRRL B-24552 contig28.1, whole genome
shotgun sequence; 759944490; NZ_JOAG01000030.1
4412; Nonomuraea Candida strain NRRL B-24552 contig42.1, whole genome
shotgun sequence; 759948103; NZ_JOAG01000045.1
4413; Streptomyces fulvissimus DSM 40593, complete genome; 488607535;
NC_021177.1
4414; Microcystis aeruginosa PCC 9807, whole genome shotgun sequence;
425454132; NZ_HE973326.1
4415; Streptomyces natalensis ATCC 27448 Scaffold_46, whole genome
shotgun sequence; 764442321; NZ_JRKI01000041.1
4416; Streptomyces iranensis genome assembly Siranensis, scaffold
SCAF00002; 765016627; NZ_LK022849.1
4417; Risungbinella massiliensis strain GD1, whole genome shotgun sequence;
765315585; NZ_LN812103.1
4418; Paenibacillus terrae strain NRRL B-30644 contig00007, whole genome
shotgun sequence; 765319397; NZ_JTHP01000007.1
4419; Streptococcus suis strain LS8I, whole genome shotgun sequence;
766595491; NZ_CEHM01000004.1
4420; Bacillus mycoides strain 11kri323 LG56_082, whole genome shotgun
sequence; 765533368; NZ_JYCJ01000082.1
4421; Paenibacillus polymyxa strain NRRL B-30509 contig00003, whole
genome shotgun sequence; 766607514; NZ_JTHO01000003.1
4422; Frankia sp. CpI1-P FF86 1013, whole genome shotgun sequence;
946950294; NZ_LJJX01000013.1
4423; Streptococcus suis strain B28P, whole genome shotgun sequence;
769231516; NZ_CDTB01000010.1
4424; Lachnospiraceae bacterium NK4A144
G619DRAFT_scaffold00002.2_C, whole genome shotgun sequence;
652826657; NZ_AUJT01000002.1
4425; Lechevalieria aerocolonigenes strain NRRL B-16140 contig11.3, whole
genome shotgun sequence; 772744565; NZ_JYJG01000059.1
4426; Streptomyces sp. NRRL F-4428 contig40.2, whole genome shotgun
sequence; 772774737; NZ_JYJI01000131.1
4427; Streptomyces sp. FxanaA7 F611DRAFT_scaffold00041.41_C, whole
genome shotgun sequence; 780340655; NZ_LACL01000054.1
4428; Streptomyces rubellomurinus strain ATCC 31215 contig-63, whole
genome shotgun sequence; 783211546; NZ_JZKH01000064.1
4429; Streptomyces rubellomurinus subsp. indigofems strain ATCC 31304
contig-55, whole genome shotgun sequence; 783374270;
NZ_JZKG01000056.1
4430; Elstera litoralis strain Dia-1 c21, whole genome shotgun sequence;
788026242; NZ_LAJY01000021.1
4431; Streptomyces sp. NRRL B-1568 contig-76, whole genome shotgun
sequence; 799161588; NZ_JZWZ01000076.1
4432; Sphingomonas sp. SRS2 contig40, whole genome shotgun sequence;
806905234; NZ_LARW01000040.1
4433; Paenibacillus wulumuqiensis strain Y24 Scaffold4, whole genome
shotgun sequence; 808051893; NZ_KQ040793.1
4434; Paenibacillus dauci strain H9 Scaffold3, whole genome shotgun
sequence; 808064534; NZ_KQ040798.1
4435; Spirosoma radiotolerans strain DG5A, complete genome; 817524426;
NZ_CP010429.1
4436; Allosalinactinospora lopnorensis strain CA15-2 contig00044, whole
genome shotgun sequence; 815863894; NZ_LAJC01000044.1
4437; Allosalinactinospora lopnorensis strain CA15-2 contig00053, whole
genome shotgun sequence; 815864238; NZ_LAJC01000053.1
4438; Bacillus sp. SA1-12 scf7180000003378, whole genome shotgun
sequence; 817541164; NZ_LATZ01000026.1
4439; Altererythrobacter atlanticus strain 26DY36, complete genome;
927872504; NZ_CP011452.2
4440; Streptomyces lydicus A02, complete genome; 822214995;
NZ_CP007699.1
4441; Streptomyces lydicus A02, complete genome; 822214995;
NZ_CP007699.1
4442; Bacillus census strain B4147 NODE_5, whole genome shotgun
sequence; 822530609; NZ_LCYN01000004.1
4443; Erythrobacter luteus strain KA37 contig1, whole genome shotgun
sequence; 822631216; NZ_LBHB01000001.1
4444; Erythrobacter marinus strain HWDM-33 contig3, whole genome
shotgun sequence; 823659049; NZ_LBHU01000003.1
4445; Streptomyces sp. KE1 Contig11, whole genome shotgun sequence;
825353621; NZ_LAYX01000011.1
4446; Sphingomonas sp. Y57 scaffold74, whole genome shotgun sequence;
826051019; NZ_LDES01000074.1
4447; Alistipes sp. ZOR0009 L990_140, whole genome shotgun sequence;
835319962; NZ_JTLD01000119.1
4448; Bacillus aryabhattai strain T61 Scaffold1, whole genome shotgun
sequence; 836596561; NZ_KQ087173.1
4449; Paenibacillus sp. TCA20, whole genome shotgun sequence; 843088522;
NZ_BBIW01000001.1
4450; Bacillus circulans strain RIT379 contig11, whole genome shotgun
sequence; 844809159; NZ_LDPH01000011.1
4451; Bacillus circulans strain RIT379 contig11, whole genome shotgun
sequence; 844809159; NZ_LDPH01000011.1
4452; Ornithinibacillus californiensis strain DSM 16628 contig_22, whole
genome shotgun sequence; 849059098; NZ_LDUE01000022.1
4453; Bacillus pseudalcaliphilus strain DSM 8725 super11, whole genome
shotgun sequence; 849078078; NZ_LFJO01000006.1
4454; Bacillus aryabhattai strain LK25 16, whole genome shotgun sequence;
850356871; NZ_LDWN01000016.1
4455; Methanobacterium formicicum genome assembly DSM1535,
chromosome: chrI; 851114167; NZ_LN515531.1
4456; Methanobacterium arcticum strain M2
EI99DRAFT_scaffold00005.5_C, whole genome shotgun sequence;
851140085; NZ_JQKN01000008.1
4457; Methanobacterium sp. SMA-27 DL91DRAFT_nitig_0_quiver.1_C,
whole genome shotgun sequence; 851351157; NZ_JQLY01000001.1
4458; Cellulomonas sp. A375-1 contig_129, whole genome shotgun sequence;
856992287; NZ_LFKW01000127.1
4459; Streptomyces sp. HNS054 contig28, whole genome shotgun sequence;
860547590; NZ_LDZX01000028.1
4460; Bacillus census strain RIMV BC 126 212, whole genome shotgun
sequence; 872696015; NZ_LABO01000035.1
4461; Streptomyces leeuwenhoekii strain C58 contig126, whole genome
shotgun sequence; 873282818; NZ_LFEH01000123.1
4462; Bacillus sp. 220 BSPC 1447_75439_1072255, whole genome shotgun
sequence; 880954155; NZ_JVPL01000109.1
4463; Bacillus sp. 522_BSPC 2470_72498_1083579_594_{——}. . ._522_, whole
genome shotgun sequence; 880997761; NZ_JVDT01000118.1
4464; Bacillus sp. 522_BSPC 2470_72498_1083579_594_{——}. . ._522_, whole
genome shotgun sequence; 880997761; NZ_JVDT01000118.1
4465; Streptomyces varsoviensis strain NRRL B-3589 contig2.1, whole
genome shotgun sequence; 664348063; NZ_JOFN01000002.1
4466; Scytonema tolypothrichoides VB-61278 scaffold_6, whole genome
shotgun sequence; 890002594; NZ_JXCA01000005.1
4467; Erythrobacter atlanticus strain s21-N3, complete genome; 890444402;
NZ_CP011310.1
4468; Streptococcus pseudopneumoniae strain 445_SPSE
347_91401_2272315_318_{——}. . ._319_, whole genome shotgun sequence;
896667361; NZ_JVGV01000030.1
4469; Kitasatospora sp. MY 5-36 Contig_703_, whole genome shotgun
sequence; 902792184; NZ_LFVW01000692.1
4470; Streptomyces caatingaensis strain CMAA 1322 contig02, whole genome
shotgun sequence; 906344334; NZ_LFXA01000002.1
4471; Streptomyces caatingaensis strain CMAA 1322 contig07, whole genome
shotgun sequence; 906344339; NZ_LFXA01000007.1
4472; Streptomyces caatingaensis strain CMAA 1322 contig09, whole genome
shotgun sequence; 906344341; NZ_LFXA01000009.1
4473; Candidatus Halobonum tyrrellensis G22 contig00002, whole genome
shotgun sequence; 557371823; NZ_ASGZ01000002.1
4474; Bacillus weihenstephanensis strain JAS 83/3 Bw_JAS-
83/3_contig00005, whole genome shotgun sequence; 910095435;
NZ_JNLY01000005.1
4475; Silvibacterium bohemicum strain S15 contig_3, whole genome shotgun
sequence; 910257956; NZ_LBHJ01000003.1
4476; Silvibacterium bohemicum strain S15 contig_3, whole genome shotgun
sequence; 910257956; NZ_LBHJ01000003.1
4477; Silvibacterium bohemicum strain S15 contig_30, whole genome shotgun
sequence; 910257973; NZ_LBHJ01000020.1
4478; Xanthomonas campestris pv. viticola strain LMG 965, whole genome
shotgun sequence; 704493846; NZ_CBZT010000006.1
4479; Streptomyces baamensis strain NRRL B-2842 P144_Doro1_scaffold6,
whole genome shotgun sequence; 662129456; NZ_KL573544.1
4480; Streptomyces albus subsp. albus strain NRRL B-2445 contig1.1, whole
genome shotgun sequence; 664084661; NZ_JOED01000001.1
4481; Bacillus flexus strain Riq5 contig_32, whole genome shotgun sequence;
914730676; NZ_LFQJ01000032.1
4482; Salinibacter ruber M8 chromosome, complete genome; 294505815;
NC_014032.1
4483; Streptomyces vitaminophilus DSM 41686
A3IGDRAFT_scaffold_10.11, whole genome shotgun sequence; 483682977;
NZ_KB904636.1
4484; Halomonas anticariensis FP35 = DSM 16096 strain FP35 Scaffold1,
whole genome shotgun sequence; 514429123; NZ_KE332377.1
4485; Cohnella thermotolerans DSM 17683 G485DRAFT_scaffold00003.3,
whole genome shotgun sequence; 652794305; NZ_KE386956.1
4486; Streptomyces sp. GXT6 genomic scaffold Scaffold4, whole genome
shotgun sequence; 654975403; NZ_KI601366.1
4487; Verrucomicrobia bacterium LP2A
G346DRAFT_scf7180000000012_quiver.2_C, whole genome shotgun
sequence; 640169055; NZ_JAFS01000002.1
4488; Actinomadura oligospora ATCC 43269
P696DRAFT_scaffold00008.8_C, whole genome shotgun sequence;
651281457; NZ_JADG01000010.1
4489; Actinomadura oligospora ATCC 43269
P696DRAFT_scaffold00008.8_C, whole genome shotgun sequence;
651281457; NZ_JADG01000010.1
4490; Rubellimicrobium mesophilum DSM 19309 scaffold23, whole genome
shotgun sequence; 739419616; NZ_KK088564.1
4491; Pseudonocardia acaciae DSM 45401 N912DRAFT_scaffold00002.2_C,
whole genome shotgun sequence; 655569633; NZ_JIAI01000002.1
4492; Terasakiella pusilia DSM 6293 Q397DRAFT_scaffold00039.39_C,
whole genome shotgun sequence; 655499373; NZ_JHYO01000039.1
4493; Bacillus sp. MB2021 T349DRAFT_scaffold00010.10_C, whole
genome shotgun sequence; 671553628; NZ_JNJJ01000011.1
4494; Streptomyces olindensis strain DAUFPE 5622 103, whole genome
shotgun sequence; 739918964; NZ_JJOH01000097.1
4495; Thioclava dalianensis strain DLFJ1-1 contig2, whole genome shotgun
sequence; 740220529; NZ_JHEH01000002.1
4496; Streptomyces megasporus strain NRRL B-16372 contig19.1, whole
genome shotgun sequence; 671525382; NZ_JODL01000019.1
4497; Streptomyces achromogenes subsp. achromogenes strain NRRL B-2120
contig2.1, whole genome shotgun sequence; 664063830;
NZ_JODT01000002.1
4498; Microbispora rosea subsp. nonnitritogenes strain NRRL B-2631
contig12.1, whole genome shotgun sequence; 663732121;
NZ_JNZQ01000012.1
4499; Streptomyces sp. NRRL S-920 contig36.1, whole genome shotgun
sequence; 664256887; NZ_JODF01000036.1
4500; Streptomyces flavochromogenes strain NRRL B-2684 contig8.1, whole
genome shotgun sequence; 663317502; NZ_JNZO01000008.1
4501; Streptomyces natalensis strain NRRL B-5314 P055_Doro1_scaffold13,
whole genome shotgun sequence; 662108422; NZ_KL570019.1
4502; Bacillus sp. UNC322MFChir4.1 BR72DRAFT_scaffold00004.4, whole
genome shotgun sequence; 737456981; NZ_KN050811.1
4503; Paenibacillus wynnii strain DSM 18334 unitig_2, whole genome
shotgun sequence; 738760618; NZ_JQCR01000002.1
4504; Amycolatopsis sp. MJM2582 contig00007, whole genome shotgun
sequence; 739487309; NZ_JPLW01000007.1
4505; Sphingopyxis fribergensis strain Kp5.2, complete genome; 749188513;
NZ_CP009122.1
4506; Brevundimonas nasdae strain TPW30 Contig_11, whole genome
shotgun sequence; 746187486; NZ_JWSY01000011.1
4507; Microcystis panniformis FACHB-1757, complete genome; 917764592;
NZ_CP011339.1
4508; Desulfocapsa sulfexigens DSM 10523, complete genome; 451945650;
NC_020304.1
4509; Gorillibacterium massiliense strain G5, whole genome shotgun
sequence; 750677319; NZ_CBQR020000171.1
4510; Salinarimonas rosea DSM 21201 G407DRAFT_scaffold00021.21_C,
whole genome shotgun sequence; 655990125; NZ_AUBC01000024.1
4511; Streptomyces sp. NRRL S-118 P205_Doro1_scaffold34, whole genome
shotgun sequence; 664565137; NZ_KL591029.1
4512; Streptomyces glaucescens strain GLA.O, complete genome; 759802587;
NZ_CP009438.1
4513; Paenibacillus sp. FSL R5-0912, complete genome; 754884871;
NZ_CP009282.1
4514; Paenibacillus sp. FSL P4-0081, complete genome; 754777894;
NZ_CP009280.1
4515; Bacillus subtilis subsp. spizizenii RFWG1A4 contig00010, whole
genome shotgun sequence; 764657375; NZ_AJHM01000010.1
4516; Paenibacillus algorifonticola strain XJ259 Scaffold20_1, whole genome
shotgun sequence; 808072221; NZ_LAQO01000025.1
4517; Mycobacterium sp. UM_Kg27 contig000002, whole genome shotgun
sequence; 809025315; NZ_JRMM01000002.1
4518; Mycobacterium sp. UM_Kg1 contig000164, whole genome shotgun
sequence; 809073490; NZ_JRMK01000164.1
4519; Streptomyces avicenniae strain NRRL B-24776 contig3.1, whole
genome shotgun sequence; 919531973; NZ_JOEK01000003.1
4520; Paenibacillus peoriae strain HS311, complete genome; 922052336;
NZ_CP011512.1
4521; Paenibacillus sp. FJAT-27812 scaffold_0, whole genome shotgun
sequence; 922780240; NZ_LIGH01000001.1
4522; Hapalosiphon sp. MRB220 contig_91, whole genome shotgun
sequence; 923076229; NZ_LIRN01000111.1
4523; Bacillus sp. FJAT-21352 Scaffold1, whole genome shotgun sequence;
924654439; NZ_LIUS01000003.1
4524; Bacillus gobiensis strain FJAT-4402 chromosome; 926268043;
NZ_CP012600.1
4525; Streptomyces sp. NRRL B-1140 P439contig15.1, whole genome
shotgun sequence; 926344107; NZ_LGEA01000058.1
4526; Streptomyces sp. NRRL B-1140 P439contig32.1, whole genome
shotgun sequence; 926344331; NZ_LGEA01000105.1
4527; Streptomyces sp. NRRL F-5755 P309contig48.1, whole genome
shotgun sequence; 926371517; NZ_LGCW01000271.1
4528; Streptomyces sp. NRRL F-5755 P309contig50.1, whole genome
shotgun sequence; 926371520; NZ_LGCW01000274.1
4529; Saccharothrix sp. NRRL B-16348 P442contig71.1, whole genome
shotgun sequence; 926395199; NZ_LGED01000246.1
4530; Streptomyces sp. WM6378 P402contig63.1, whole genome shotgun
sequence; 926403453; NZ_LGDD01000321.1
4531; Streptomyces sp. WM6378 P402contig63.1, whole genome shotgun
sequence; 926403453; NZ_LGDD01000321.1
4532; Nocardia sp. NRRL S-836 P437contig3.1b, whole genome shotgun
sequence; 926412094; NZ_LGDY01000103.1
4533; Nocardia sp. NRRL S-836 P437contig39.1, whole genome shotgun
sequence; 926412104; NZ_LGDY01000113.1
4534; Paenibacillus sp. A59 contig_353, whole genome shotgun sequence;
927084730; NZ_LITU01000050.1
4535; Paenibacillus sp. A59 contig_416, whole genome shotgun sequence;
927084736; NZ_LITU01000056.1
4536; Streptomyces sp. NRRL S-444 contig322.4, whole genome shotgun
sequence; 797049078; JZWX01001028.1
4537; Streptomyces chattanoogensis strain NRRL ISP-5002 ISP5002contig8.1,
whole genome shotgun sequence; 928897585; NZ_LGKG01000196.1
4538; Streptomyces chattanoogensis strain NRRL ISP-5002 ISP5002contig9.1,
whole genome shotgun sequence; 928897596; NZ_LGKG01000207.1
4539; Bacillus sp. FJAT-28004 scaffold_2, whole genome shotgun sequence;
929005248; NZ_LGHP01000003.1
4540; Actinobacteria bacterium OK074 ctg60, whole genome shotgun
sequence; 930473294; NZ_LJCV01000275.1
4541; Actinobacteria bacterium OK006 ctg112, whole genome shotgun
sequence; 930490730; NZ_LJCU01000014.1
4542; Actinobacteria bacterium OK006 ctg96, whole genome shotgun
sequence; 930491003; NZ_LJCU01000287.1
4543; Kibdelosporangium phytohabitans strain KLBMP1 111, complete
genome; 931609467; NZ_CP012752.1
4544; Paenibacillus sp. GD6, whole genome shotgun sequence; 939708098;
NZ_LN831198.1
4545; Paenibacillus sp. GD6, whole genome shotgun sequence; 939708105;
NZ_LN831205.1
4546; Alicyclobacillus ferrooxydans strain TC-34 contig_22, whole genome
shotgun sequence; 940346731; NZ_LJCO01000107.1
4547; Streptomyces pactum strain ACT12 scaffold1, whole genome shotgun
sequence; 943388237; NZ_LIQD01000001.1
4548; Streptomyces flocculus strain NRRL B-2465 B2465 contig_205, whole
genome shotgun sequence; 943674269; NZ_LIQO01000205.1
4549; Streptomyces aurantiacus strain NRRL ISP-5412 ISP-5412_contig_138,
whole genome shotgun sequence; 943881150; NZ_LIPP01000138.1
4550; Streptomyces graminilatus strain NRRL B-59124 B59124_contig_7,
whole genome shotgun sequence; 943897669; NZ_LIQQ01000007.1
4551; Streptomyces alboniger strain NRRL B-1832 B-1832_contig_37, whole
genome shotgun sequence; 943898694; NZ_LIQN01000037.1
4552; Streptomyces alboniger strain NRRL B-1832 B-1832_contig_384,
whole genome shotgun sequence; 943899498; NZ_LIQN01000384.1
4553; Streptomyces kanamyceticus strain NRRL B-2535 B-2535_contig_122,
whole genome shotgun sequence; 943922224; NZ_LIQU01000122.1
4554; Streptomyces kanamyceticus strain NRRL B-2535 B-2535_contig_247,
whole genome shotgun sequence; 943922567; NZ_LIQU01000247.1
4555; Streptomyces luridiscabiei strain NRRL B-24455 B24455_contig_315,
whole genome shotgun sequence; 943927948; NZ_LIQV01000315.1
4556; Streptomyces atriruber strain NRRL B-24165 contig_124, whole
genome shotgun sequence; 943949281; NZ_LIPN01000124.1
4557; Streptomyces hirsutus strain NRRL B-2713 B2713 contig_57, whole
genome shotgun sequence; 944005810; NZ_LIQT01000057.1
4558; Streptomyces aureus strain NRRL B-2808 contig_171, whole genome
shotgun sequence; 944012845; NZ_LIPQ01000171.1
4559; Streptomyces prasinus strain NRRL B-12521 B12521_contig_230,
whole genome shotgun sequence; 944020089; NZ_LIPR01000230.1
4560; Streptomyces phaeochromogenes strain NRRL B-1248 B-
1248_contig_126, whole genome shotgun sequence; 944029528;
NZ_LIQZ01000126.1
4561; Streptomyces prasinus strain NRRL B-2712 B2712_contig_323, whole
genome shotgun sequence; 944410649; NZ_LIRH01000323.1
4562; Streptomyces prasinopilosus strain NRRL B-2711 B2711_contig_370,
whole genome shotgun sequence; 944415035; NZ_LIRG01000370.1
4563; Streptomyces torulosus strain NRRL B-3889 B-3889_contig_18, whole
genome shotgun sequence; 944495433; NZ_LIRK01000018.1
4564; Frankia alni str. ACN14A chromosome, complete sequence;
111219505; NC_008278.1
4565; Paenibacillus sp. Leaf72 contig_6, whole genome shotgun sequence;
947378267; NZ_LMLV01000032.1
4566; Sanguibacter sp. Leaf3 contig_2, whole genome shotgun sequence;
947472882; NZ_LMRH01000002.1
4567; Aeromicrobium sp. Root344 contig_1, whole genome shotgun
sequence; 947552260; NZ_LMDH01000001.1
4568; Sphingopyxis sp. Root1497 contig_3, whole genome shotgun sequence;
947689975; NZ_LMGF01000003.1
4569; Sphingopyxis sp. Root1497 contig_3, whole genome shotgun sequence;
947689975; NZ_LMGF01000003.1
4570; Sphingomonas sp. Root1294 contig_7, whole genome shotgun
sequence; 947890193; NZ_LMEJ01000014.1
4571; Sphingomonas sp. Root720 contig_7, whole genome shotgun sequence;
947704642; NZ_LMID01000015.1
4572; Sphingomonas sp. Root720 contig_8, whole genome shotgun sequence;
947704650; NZ_LMID01000016.1
4573; Sphingomonas sp. Root710 contig_1, whole genome shotgun sequence;
947721816; NZ_LMIB01000001.1
4574; Mesorhizobium sp. Root172 contig_2, whole genome shotgun sequence;
947919015; NZ_LMHP01000012.1
4575; Mesorhizobium sp. Root102 contig_3, whole genome shotgun sequence;
947937119; NZ_LMCP01000023.1
4576; Paenibacillus sp. Soil750 contig_1, whole genome shotgun sequence;
947966412; NZ_LMSD01000001.1
4577; Paenibacillus sp. Soil750 contig_1, whole genome shotgun sequence;
947966412; NZ_LMSD01000001.1
4578; Paenibacillus sp. Soil522 contig_3, whole genome shotgun sequence;
947983982; NZ_LMRV01000044.1
4579; Paenibacillus sp. Root52 contig_3, whole genome shotgun sequence;
948045460; NZ_LMFO01000023.1
4580; Bacillus sp. Soil768D1 contig_5, whole genome shotgun sequence;
950170460; NZ_LMTA01000046.1
4581; Paenibacillus sp. Soil724D2 contig_11, whole genome shotgun
sequence; 946400391; LMRY01000003.1
4582; Paenibacillus sp. Root444D2 contig_4, whole genome shotgun
sequence; 950271971; NZ_LMEO01000034.1
4583; Paenibacillus sp. Soil766 contig_32, whole genome shotgun sequence;
950280827; NZ_LMSJ01000026.1
4584; Paenibacillus sp. Soil766 contig_32, whole genome shotgun sequence;
950280827; NZ_LMSJ01000026.1
4585; Streptomyces sp. Root1310 contig_5, whole genome shotgun sequence;
951121600; NZ_LMEQ01000031.1
4586; Bacillus muralis strain DSM 16288 Scaffold4, whole genome shotgun
sequence; 951610263; NZ_LMBV01000004.1
4587; Streptomyces sp. MBT76 scaffold_4, whole genome shotgun sequence;
953813790; NZ_LNBE01000004.1
4588; Gorillibacterium sp. SN4, whole genome shotgun sequence; 960412751;
NZ_LN881722.1
4589; Thalassobius activus strain CECT 5114, whole genome shotgun
sequence; 960424655; NZ_CYUE01000025.1
4590; Microbacterium testaceum strain NS283 contig_37, whole genome
shotgun sequence; 969836538; NZ_LDRU01000037.1
4591; Microbacterium testaceum strain NS206 contig_27, whole genome
shotgun sequence; 969912012; NZ_LDRS01000027.1
4592; Microbacterium testaceum strain NS183 contig_65, whole genome
shotgun sequence; 969919061; NZ_LDRR01000065.1
4593; Sphingopyxis sp. H050 H050_contig000006, whole genome shotgun
sequence; 970555001; NZ_LNRZ01000006.1

What is claimed:

1. A fusion protein comprising a bacteriophage coat protein fused to a lasso peptide component.

2. The fusion protein of claim 1, wherein the bacteriophage coat protein comprises p3, p6, p7, p8 or p9 of filamentous phages, small outer capsid (SOC) protein or highly antigenic outer capsid (HOC) protein of a T4 phage, pX of a T7 phage, pD or pV of a λ (lambda) phage or a functional variant thereof.

3. The fusion protein of claim 2, wherein the functional variant is selected from a truncation, deletion, insertion, mutation, conjugation, domain-shuffling or domain-swapping.

4. The fusion protein of claim 1, wherein the lasso peptide component is a lasso precursor peptide, a lasso core peptide, a lasso peptide or a functional fragment of lasso peptide.

5. The fusion protein of claim 4, wherein the lasso precursor peptide comprises a sequence of any one of the even numbers of SEQ ID NOS:1-2630, or a sequence having greater than 30% identity of any one of the even numbers of SEQ ID NOS:1-2630.

6. The fusion protein of claim 1, wherein the fusion protein further comprises a periplasmic secretion signal.

7. The fusion protein of claim 6, wherein the periplasmic secretion signal is a periplasmic space-targeting signal sequence derived from TorA, PelB, OmpA, pi, PhoA, DsbA, TolB, TorT, a substrate of the Type II Secretion System (T2SS), or a functional variant thereof.

8. The fusion protein of claim 1, wherein the bacteriophage coat protein is fused to the lasso peptide component via a first linker.

9. The fusion protein of claim 8, wherein the first linker is a cleavable linker.

10. The fusion protein of any one of claims 1 to 10, wherein the lasso peptide fragment comprises at least one unusual amino acid or unnatural amino acid.

11. A nucleic acid molecule encoding the fusion protein according to any one of claims 1 to 10.

12. The nucleic acid molecule of claim 11, wherein the nucleic acid comprises a sequence of any one of the odd numbers of SEQ ID NOS:1-2630, or a sequence having greater than 30% identity of any one of the odd numbers of SEQ ID NOS:1-2630.

13. The fusion protein of claim 10 or 12, wherein the nucleic acid molecule is a phagemid.

14. The fusion protein of any one of claims 1 to 13, wherein the bacteriophage coat protein is derived from a filamentous bacteriophage, a polyhedral bacteriophage, a tailed bacteriophage, or a pleomorphic bacteriophage.

15. The fusion protein of any one of claims 1 to 15, wherein the bacteriophage coat protein is derived from an M13 phage, T4 phage, T7 phage or λ (lambda) phage.

16. A fusion protein comprising at least one lasso peptide biosynthesis component fused to a secretion signal.

17. The fusion protein of claim 16, wherein the secretion signal is a periplasmic secretion signal.

18. The fusion protein of claim 17, wherein the periplasmic secretion signal is a periplasmic space targeting signal sequence derived from TorA, PelB, OmpA, pIII, PhoA, DsbA, TolB, TorT, a substrate of the Type II Secretion System (T2SS), or a functional variant thereof.

19. The fusion protein of claim 16, wherein the secretion signal is an extracellular secretion signal.

20. The fusion protein of claim 19, wherein the extracellular secretion signal is an extracellular space targeting signal sequence derived from HlyA, a substrate of the Type 1 Secretion System (T1 SS), or a functional variant thereof.

21. The fusion protein of any one of claims 16 to 20, wherein the at least one lasso peptide biosynthesis component is a lasso peptidase, a lasso cyclase or a lasso RiPP Recognition Element (RRE).

22. The fusion protein of claim 21, wherein the lasso peptidase comprises a sequence of any one of peptide Nos: 1316-2336, or a sequence having greater than 30% identity of any one of peptide Nos: 1316-2336.

23. The fusion protein of claim 21 or 22, wherein the lasso cyclase comprises a sequence of any one of peptide Nos: 2337-3761, or a sequence having greater than 30% identity of any one of peptide Nos: 2337-3761.

24. The fusion protein of any one of claim 21 to 23, wherein the lasso RRE comprises a sequence of any one of peptide Nos: 3762-4593, or a sequence having greater than 30% identity of any one of peptide Nos: 3762-4593.

25. The fusion protein of any one of claims 16 to 21, wherein the fusion protein comprises the lasso peptidase and the lasso RRE.

26. The fusion protein of claim 25, wherein the fusion protein comprises a sequence of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562, or a sequence having greater than 30% identity of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562.

27. The fusion protein of any one of claims 16 to 21, wherein the fusion protein comprises the lasso cyclase and the lasso RRE.

28. The fusion protein of claim 27, wherein the fusion protein comprises a sequence selected from peptide Nos: 2504, 3608 or a sequence having greater than 30% identity of any one of peptide Nos: 2504 and 3608.

29. The fusion protein of any one of claims 16 to 21, wherein the fusion protein comprises the lasso peptidase and the lasso cyclase.

30. The fusion protein of claim 29, wherein the fusion protein comprises a sequence having peptide No: 2903 or a sequence having greater than 30% identity thereof.

31. The fusion protein of any one of claims 16 to 21, wherein the fusion protein comprises the lasso peptidase, the lasso cyclase and the lasso RRE.

32. The fusion protein of any one of claims 16 to 21, wherein the fusion protein comprises more than one lasso peptide biosynthesis component fused together via a first cleavable linker.

33. The fusion protein of any one of claims 16 to 32, wherein the lasso peptide biosynthesis component is fused to the secretion signal via a second cleavable linker.

34. A nucleic acid molecule encoding the fusion protein according to any one of claims 16 to 33.

35. The nucleic acid molecule of claim 34, wherein the nucleic acid comprises a sequence encoding any one of peptide Nos. 1316-2336, 2337-3761, and 3762-4593.

36. A system comprising (i) a first nucleic acid sequence encoding one or more structural proteins of a bacteriophage; (ii) a second nucleic acid sequence encoding at least one lasso peptide component; and (iii) a third nucleic acid sequence encoding at least one lasso peptide biosynthesis component.

37. The system according to claim 36, wherein the first nucleic acid sequence is one or more plasmid.

38. The system according to claim 36 or 37, wherein the bacteriophage is an M13 phage, a fd phage or a fl phage.

39. The system according to claim 36, wherein the first nucleic acid sequence encodes one or more of p3, p6, p7, p8 or p9 of filamentous phages, or a functional variant thereof.

40. The system according to any one of 36 to 39, wherein the third nucleic acid sequence encodes one or more fusion protein each comprising at least one lasso peptide biosynthesis component fused to a (a) first secretion signal or (b) purification tag.

41. The system according to claim 40, wherein the at least one lasso peptide biosynthesis component comprises one or more of a lasso peptidase, a lasso cyclase and a lasso RRE.

42. The system according to claim 40, wherein the third nucleic acid sequence encodes a first fusion protein comprising a lasso peptidase and the (a) first secretion signal or (b) purification tag.

43. The system according to claim 42, wherein the third nucleic acid sequence further encodes a second fusion protein comprising a lasso cyclase and the (a) first secretion signal or (b) purification tag.

44. The system according to claim 43, wherein the third nucleic acid sequence further encodes a third fusion protein comprising a lasso RRE and the (a) first secretion signal or (b) purification tag.

45. The system according to claim 40, wherein the third nucleic acid sequence encodes a first fusion protein comprising a lasso peptidase, a lasso cyclase and the (a) first secretion signal or (b) purification tag.

46. The system according to claim 45, wherein the third nucleic acid sequence further encodes a second fusion protein comprising an RRE and the (a) first secretion signal or (b) purification tag.

47. The system according to claim 40, wherein the third nucleic acid sequence encodes a first fusion protein comprising a lasso peptidase, a lasso RRE and the (a) first secretion signal or (b) purification tag.

48. The system according to claim 47, wherein the third nucleic acid sequence further encodes a second fusion protein comprising a lasso cyclase and the (a) first secretion signal or (b) purification tag.

49. The system according to claim 40, wherein the third nucleic acid sequence encodes a first fusion protein comprising a lasso cyclase, a lasso RRE and the (a) first secretion signal or (b) purification tag.

50. The system according to claim 49, wherein the third nucleic acid sequence further encodes a second fusion protein comprising a lasso peptidase and the (a) first secretion signal or (b) purification tag.

51. The system according to claim 40, wherein the third nucleic acid sequence encodes a fusion protein comprising a lasso peptidase, a lasso cyclase, a lasso RRE and the (a) first secretion signal or (b) purification tag.

52. The system according to any one of claims 36 to 51, wherein the first secretion signal is a periplasmic secretion signal.

53. The system according to any one of claims 36 to 52, wherein the first secretion signal is an extracellular secretion signal.

54. The system according to any one of claims 36 to 53, wherein the third nucleic acid sequence is one or more plasmid.

55. The system according to any one of claims 36 to 54, wherein the second nucleic acid sequence encodes a fourth fusion protein comprising a lasso peptide component, a bacteriophage coat protein and a second secretion signal, and wherein the second secretion signal is a periplasmic secretion signal.

56. The system according to any one of claims 36 to 55, wherein the lasso peptide component is a lasso precursor peptide, a lasso core peptide, a lasso peptide or a functional fragment of lasso peptide.

57. The system according to claim 55 or 56, wherein the lasso precursor peptide or the lasso core peptide is fused to the bacteriophage coat protein via a cleavable linker.

58. The system according to any one of claims 55 to 57, wherein the bacteriophage coat protein comprises p3, p6, p8 or p9 of filamentous phages, or a functional variant thereof.

59. The system according to any one of claims 55 to 58, wherein the second nucleic acid sequence is a plasmid or a phagemid.

60. The system according to any one of claims 36 to 59, wherein the second nucleic acid sequence comprises a sequence of (i) any one of the odd numbers of SEQ ID NOS:1-2630, (ii) a sequence having greater than 30% identity of any one of the odd numbers of SEQ ID NOS:1-2630, or (iii) a sequence encoding a polypeptide having greater than 30% identity of any one of the even numbers of SEQ ID NOS:1-2630.

61. The system according to any one of claims 36 to 60, wherein the third nucleic acid sequence comprises a sequence encoding a polypeptide having greater than 30% identify of any one of peptide Nos: 1316-2336, peptide Nos: 2337-3761, and peptide Nos: 3762-4593.

62. The system according to any one of claims 36 to 61, wherein two or more of the first nucleic acid sequence, the second nucleic acid sequence and the third nucleic acid sequence are in the same nucleic acid molecule.

63. The system according to claim 62, wherein the nucleic acid molecule is a phagemid.

64. The system according to any one of claims 36 to 63, wherein the periplasmic secretion signal is a periplasmic space targeting signal sequence derived from TorA, PelB, OmpA, pIII, PhoA, DsbA, TolB, TorT, a substrate of the Type II Secretion System (T2SS), or a functional variant thereof.

65. The system according to any one of claims 36 to 64, wherein the extracellular secretion signal is an extracellular space targeting signal sequence derived from HlyA or a substrate of the Type 1 Secretion System (T1SS), or a functional variant thereof.

66. The system according to any one of claims 36 to 65, wherein the purification tag is Albumin-binding protein (ABP), Alkaline Phosphatase (AP), AU1 epitope, AU5 epitope, Bacteriophage T7 epitope (T7 tag), Bacteriophage V5 epitope (V5 tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1-tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

67. The system according to any one of claims 36 to 66, further comprising a bacterial cell having an intracellular space, wherein the first and second nucleic acid sequences are in the intracellular space of the bacterial cell.

68. The system according to claim 67, wherein the third nucleic acid sequence is in the intracellular space of the bacterial cell.

69. The system according to claim 68, wherein the bacterial cell further comprises a periplasmic space, and wherein the at least one lasso peptide biosynthesis component encoded by the third nucleic acid sequence is in the periplasmic space or the extracellular space.

70. The system according to claim 67, wherein the third nucleic acid sequence is not in the intracellular space of the bacterial cell.

71. The system according to any one of claims 67 to 70, wherein the bacterial cell is a cell of E. coli.

72. The system according to any one of claims 67-71, wherein the lasso peptide fragment comprises at least one unusual amino acid or unnatural amino acid.

73. A non-naturally existing bacteriophage comprising a first coat protein and a phagemid, wherein the first coat protein is fused to a lasso peptide component, and wherein the phagemid encodes at least a portion of the lasso peptide component.

74. The non-naturally existing bacteriophage of claim 73, wherein the phagemid encodes a fusion protein comprising the first coat protein and the lasso peptide component.

75. The non-naturally existing bacteriophage of claim 74, wherein the fusion protein further comprises a periplasmic secretion signal.

76. The non-naturally existing bacteriophage of claim 74, wherein the fusion protein further comprises a cleavable linker.

77. The non-naturally existing bacteriophage of claim 73, wherein the first coat protein is p3, p6, p7, p8 or p9 of filamentous phages or a functional variant thereof.

78. The non-naturally existing bacteriophage of claim 73, wherein the phagemid further encodes at least one lasso peptide biosynthesis component.

79. The non-naturally existing bacteriophage of claim 78, wherein the phagemid encodes a fusion protein comprising the lasso peptide biosynthesis component and a secretion signal.

80. The non-naturally existing bacteriophage of claim 79, wherein the secretion signal is a periplasmic secretion signal or an extracellular secretion signal.

81. The non-naturally existing bacteriophage of claim 73, wherein the phagemid comprises a nucleic acid sequence of (i) any one of the odd numbers of SEQ ID NOS:1-2630, (ii) a sequence having greater than 30% identity of any one of the odd numbers of SEQ ID NOS:1-2630, or (iii) a sequence encoding a polypeptide having greater than 30% identify of any one of the even numbers of SEQ ID NOS:1-2630, peptide Nos: 1316-2336, peptide Nos: 2337-3761, and peptide Nos: 3762-4593.

82. The non-naturally existing bacteriophage of claim 73, wherein the phagemid further encodes at least one structural protein.

83. The non-naturally existing bacteriophage of claim 82, wherein the at least one structural protein comprises p3, p6, p7, p8 or p9 of filamentous phages or a functional variant thereof.

84. The non-naturally existing bacteriophage of claim 83, wherein the phage is an M13 phage.

85. The non-naturally existing bacteriophage of any one of claims 73 to 84, wherein the bacteriophage is in a culture medium of bacteria.

86. The non-naturally existing bacteriophage of claim 85, wherein the culture medium further comprises a bacterial host of the bacteriophage.

87. The non-naturally existing bacteriophage of claim 86, wherein the culture medium further comprises at least one lasso peptide biosynthesis component secreted by the bacterial host.

88. The non-naturally existing bacteriophage of claim 86 or 87, wherein the bacterial host is E. coli.

89. The non-naturally existing bacteriophage of any one of claims 73 to 84, wherein the bacteriophage is purified.

90. The non-naturally existing bacteriophage of any one of claim 89, wherein the bacteriophage is in contact with at least one lasso peptide biosynthesis component.

91. The non-naturally existing bacteriophage of claim 18, wherein the at least one lasso peptide biosynthesis component is recombinantly produced or purified.

92. The non-naturally existing bacteriophage of any one of claims 87 to 91, wherein the lasso peptide component is a lasso precursor peptide and the at least one lasso biosynthesis component comprises a lasso peptidase and a lasso cyclase.

93. The non-naturally existing bacteriophage of any one of claims 87 to 91, wherein the lasso peptide component is a lasso core peptide and the at least one lasso biosynthesis component comprises a lasso cyclase.

94. The non-naturally existing bacteriophage of claim 92 or 93, wherein the lasso biosynthesis component further comprises a lasso RRE.

95. The non-naturally existing bacteriophage of claim 94, wherein two or more of the lasso peptidase, lasso cyclase and lasso RRE are fused together.

96. The non-naturally existing bacteriophage of any one of claims 73 to 96, wherein the lasso peptide component is a lasso peptide or a functional fragment of lasso peptide.

97. The non-naturally existing bacteriophage of any one of claims 73 to 97, wherein the lasso peptide component comprises at least one unusual or unnatural amino acid.

98. The non-naturally existing bacteriophage of any one of claims 73 to 98, wherein the bacteriophage is a filamentous bacteriophage, a polyhedral bacteriophage, a tailed bacteriophage, or a pleomorphic bacteriophage.

99. A composition comprising at least two non-naturally existing bacteriophages according to any one of claims 73 to 96.

100. The composition according to claim 99, wherein the lasso peptide components of the at least two non-naturally existing bacteriophages are the same.

101. The composition according to claim 99, wherein each of the lasso peptide components of the at least two non-naturally existing bacteriophages is unique.

102. A bacteriophage display library comprising the composition of any one of claims 99 to 101.

103. A bacterial cell comprising the system according to any one of claims 36 to 66 or the non-naturally existing bacteriophage according to any one of claims 73 to 96.

104. The bacterial cell according to claim 103, wherein the bacterial cell is a cell of E. coli.

105. The bacterial cell according to claim 103 or 104, wherein the bacterial cell is a cell of genetically engineered E. coli.

106. The bacterial cell according to claim 105, wherein the genetically engineered E. coli cell comprises a nucleic acid sequence encoding a modified aminoacyl-tRNA synthetase (aaRS) capable of recognizing an unusual or unnatural amino acid residue.

107. The bacterial cell according to claim 106 further comprises a complementary tRNA that is aminoacylated by the modified aminoacyl-tRNA synthetase (aaRS).

108. A cultural medium comprising the bacterial cell according to claim 103 to 107.

109. The culture medium of claim 108, wherein the culture medium comprises natural, non-natural or unusual amino acid residues.

110. The non-naturally existing bacteriophage according to any one of claims 73 to 96, or the composition according to any one of claims 99 to 101, or the bacteriophage display library of claim 102, or the bacterial cell according to claim 103 to 107, or the cultural medium according to claim 108 or 109, in contact with a target molecule that is capable of binding to the lasso peptide component.

111. The non-naturally existing bacteriophage according to any one of claims 73 to 96, or the composition according to any one of claims 99 to 101, or the bacteriophage display library of claim 102, or the bacterial cell according to claim 103 to 107, or the cultural medium according to claim 108 or 109, wherein the target molecule is a cell surface protein or a secreted protein.

112. The non-naturally existing bacteriophage according to claim 111, wherein the cell surface protein comprises a transmembrane domain.

113. The non-naturally existing bacteriophage according to claim 111, wherein the cell surface protein does not comprise a transmembrane domain.

114. The non-naturally existing bacteriophage according to any one of claims 73 to 96, or the composition according to any one of claims 99 to 101, or the bacteriophage display library of claim 102, or the bacterial cell according to claim 103 to 107, or the cultural medium according to claim 108 or 109, wherein the target molecule is capable of modulating a cellular activity in a cell expressing the target molecule.

115. A method for making a member of a bacteriophage display library comprising
providing a system comprising (i) a first nucleic acid sequence encoding one or more structural proteins of a bacteriophage; (ii) a phagemid comprising a second nucleic acid sequence encoding a lasso peptide component fused to a bacteriophage coat protein; and (iii) a third nucleic acid sequence encoding at least one lasso peptide biosynthesis component;
introducing the system into a population of bacterial cells;
culturing the population of bacterial cells under a suitable condition to produce a plurality of bacteriophages each displaying the lasso peptide component on the coat protein; and
wherein the lasso peptide biosynthesis component processes the lasso peptide component into a lasso peptide or a functional fragment of lasso peptide.

116. The method of claim 115, wherein the bacterial cell comprises a periplasmic space, and wherein the lasso peptide component is fused to a first periplasmic secretion signal.

117. The method of claim 116, wherein the lasso peptide biosynthesis component is fused to a second periplasmic secretion signal; and wherein the lasso peptide biosynthesis component processes the lasso peptide component into the lasso peptide or functional fragment of lasso peptide in the periplasmic space.

118. The method of claim 116, wherein the lasso peptide biosynthesis component is fused to an extracellular secretion signal; and wherein the lasso peptide biosynthesis component processes the lasso peptide component into the lasso peptide or functional fragment of lasso peptide in the extracellular space.

119. A method for making a member of bacteriophage display library comprising
providing a system comprising (i) a first nucleic acid sequence encoding one or more structural proteins of a bacteriophage; and (ii) a phagemid comprising a second nucleic acid sequence encoding a lasso peptide component fused to a bacteriophage coat protein;
introducing the system into a population of bacterial cells; and
culturing the population of bacterial cells under a first suitable condition to produce a plurality of bacteriophages each displaying the lasso peptide component on the coat protein;
contacting the plurality of bacteriophages with at least one purified lasso peptide biosynthesis component under a second suitable condition to allow the lasso peptide biosynthesis component to process the lasso peptide component into a lasso peptide or functional fragment of lasso peptide.

120. The method of 119, wherein the plurality of bacteriophages are purified before the step of contacting.

121. The method of 119, wherein the contacting is performed by adding a purified lasso peptide biosynthesis component into a culture medium containing the bacteriophages.

122. The method of any one of claims 115 to 121, wherein the population of bacterial cells are cells of E. coli of one of claims 103 to 107.

123. The method of any one of claims 115 to 122, wherein the lasso peptide components of the plurality of bacteriophages are the same.

124. The method of any one of claims 115 to 122, wherein each of the lasso peptide components of the plurality of bacteriophages is unique.

125. The method of any one of claims 115 to 124, wherein the system is the system of any one of claims 36 to 71.

126. A method for evolving a lasso peptide of interest for a target property, comprising
a. providing a first bacteriophage display library comprising members derived from the lasso peptide of interest, wherein each member of the first lasso peptide display library comprises at least one mutation to the lasso peptide of interest;
b. subjecting the library to a first assay under a first condition to identify members having the target property;
c. identifying the mutations of the identified members as beneficial mutations; and
d. introducing the beneficial mutations into the lasso peptide of interest to provide an evolved lasso peptide.

127. The method of claim 126, wherein the method further comprises:
f. providing an evolved bacteriophage display library of lasso peptides comprising members derived from the evolved lasso peptide, wherein the members of the evolved bacteriophage display library retain at least one beneficial mutation;
g. repeating steps b through d.

128. The method of claim 127, wherein the method further comprises repeating steps f and g for at least one more round.

129. The method of any one of claims 126 to 128, wherein the evolved bacteriophage display library is subjected to the first assay under a second condition more stringent for the target property than the first condition.

130. The method of any one of claims 127 to 129, wherein the evolved bacteriophage display library is subjected to a second assay to identify members having the target property.

131. The method of any one of claims 126 to 130, wherein the method further comprises validating the evolved lasso peptide using at least one additional assay different from the first or second assay.

132. The method of any one of claims 126 to 131, wherein the target property comprises binding affinity for a target molecule.

133. The method of any one of claims 126 to 131, wherein the target property comprises binding specificity for a target molecule.

134. The method of any one of claims 126 to 131, wherein the target property comprises capability of modulating a cellular activity or cell phenotype.

135. The method of claim 134, wherein the modulation is antagonist modulation or agonist modulation.

136. The method of any one of claims 126 to 135, wherein the mutation comprises substituting at least one amino acid with an unusual or unnatural amino acid.

137. The method of any one of claims 126 to 136, wherein the target property is at least two target properties screened simultaneously.

138. A method for identifying a lasso peptide that specifically binds to a target molecule, the method comprising:
providing a bacteriophage display library comprising a plurality of members, each member comprising a lasso peptide or a functional fragment of lasso peptide;
contacting the library with the target molecule under a suitable condition that allows at least one member of the library to form a complex with the target molecule; and
identifying the member of in the complex.

139. The method of claim 138,
wherein the contacting is performed by contacting the library with the target molecule in the presence of a reference binding partner of the target molecule under a suitable condition that allows at least one member of the library to compete with the reference binding partner for binding to the target molecule; and
wherein the identifying step is performed by detecting reduced binding of the reference binding partner to the target molecule; and identifying the member responsible for the reduced binding.

140. The method of claim 139, wherein the reference binding partner is a ligand for the target molecule.

141. The method of claim 139 or 140, wherein the target molecule comprises one or more target sites, and the reference binding partner specifically binds to a target site of the target molecule.

142. The method of claim 140, wherein the reference binding partner is a natural ligand or synthetic ligand for the target molecule.

143. The method of any one of methods 138 to 142, wherein the target molecule is at least two target molecules.

144. A method for identifying a lasso peptide that modulates a cellular activity, the method comprising
a. providing a bacteriophage display library comprising a plurality of members, each member comprising a lasso peptide or a functional fragment of lasso peptide;
b. subjecting the library to a suitable biological assay configured for measuring the cellular activity;
c. detecting a change in the cellular activity; and
d. identifying the members responsible for the detected change.

145. The method of claim 144, wherein the step b is performed by subjecting the library to multiple biological assays configured for measuring the cellular activity; and the method further comprises selecting the members that have a high probability of being identified as responsible for the detected change in the cellular activity.

146. A method for identifying an agonist or antagonist lasso peptide for a target molecule, the method comprising:
providing a bacteriophage display library comprising a plurality of members, each member comprising a lasso peptide or a functional fragment of lasso peptide;
contacting the library with a cell expressing the target molecule under a suitable condition that allows at least one member of the library to bind to the target molecule;
measuring a cellular activity mediated by the target molecule; and
identifying the member as an agonist ligand for the target molecule if said cellular activity is increased; or identifying the member as an antagonist ligand if said cellular activity is decreased.

147. A nucleic acid molecule comprising a first sequence encoding one or more structural proteins of a bacteriophage and a second sequence encoding a first fusion protein comprising a lasso peptide component fused to a first coat protein of the bacteriophage.

148. The nucleic acid molecule of claim 147, wherein the second sequence further encodes a second fusion protein comprising an identification peptide fused to a second coat protein of the bacteriophage.

149. The nucleic acid molecule of claim 147 or 148, wherein the nucleic acid molecule is a mutated genome of the bacteriophage, wherein one or more endogenous sequence encoding the first and/or second coat protein(s) is deleted from the genome.

150. The nucleic acid molecule of any one of claims 147 to 149, wherein at least one of the first and second coat proteins is a nonessential outer capsid protein of the bacteriophage.

151. The nucleic acid molecule of claim 150, wherein the second sequence is an exogenous sequence.

152. The nucleic acid molecule of any one of claims 147 to 151, wherein the bacteriophage is a non-naturally occurring T4 phage, T7 phage or λ (lambda) phage.

153. The nucleic acid molecule of claim 152, wherein the nucleic acid molecule is a mutated genome of the T4 phage with endogenous sequences coding for HOC and/or SOC deleted.

154. The nucleic acid molecule of claim, wherein the second sequence encodes a fusion protein comprising the lasso peptide component fused to HOC.

155. The nucleic acid molecule of claim 154, wherein the second sequence encodes a fusion protein comprising the identification peptide fused to SOC.

156. The nucleic acid molecule according to any one of claims 147 to 155, wherein the lasso peptide component is a lasso precursor peptide, a lasso core peptide, a lasso peptide or a functional fragment of lasso peptide.

157. The nucleic acid molecule according to claim 156, wherein the lasso precursor peptide comprises a sequence of any one of the even numbers of SEQ ID NOS:1-2630, or a sequence having greater than 30% identity of any one of the even numbers of SEQ ID NOS:1-2630.

158. The nucleic acid molecule according to any one of claims 147 to 157, wherein the nucleic acid comprises a sequence of any one of the odd numbers of SEQ ID NOS:1-2630, or a sequence having greater than 30% identity of any one of the odd numbers of SEQ ID NOS:1-2630.

159. The nucleic acid molecule according to any one of claim 148 to 158, wherein the identification peptide is a purification tag.

160. The nucleic acid molecule according to claim 159, wherein the purification tag is Albumin-binding protein (ABP), Alkaline Phosphatase (AP), AU1 epitope, AU5 epitope, Bacteriophage T7 epitope (T7 tag), Bacteriophage V5 epitope (V5 tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1-tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

161. The nucleic acid molecule according to any one of claim 147 to 160, wherein the first fusion protein further comprises a linker between the first protein and the lasso peptide component.

162. The nucleic acid molecule according to claim 161, wherein the linker is a cleavable linker.

163. A system comprising (i) a first nucleic acid sequence encoding one or more structural proteins of a bacteriophage; (ii) a second nucleic acid sequence encoding a first fusion protein comprising a lasso peptide component fused to a first coat protein of the bacteriophage; and (iii) a third nucleic acid sequence encoding at least one lasso peptide biosynthesis component.

164. The system according to 163, wherein the second nucleic acid sequence further encodes a second fusion protein comprising an identification peptide fused to a second coat protein of the bacteriophage.

165. The system according to claim 163 or 164, wherein the first nucleic acid sequence does not encode the first and/or second nonessential outer capsid protein(s) of the bacteriophage.

166. The system according to claim 165, wherein the first nucleic acid sequence is a mutated genome of the bacteriophage.

167. The system according to claim 163 or 164, wherein the first nucleic acid sequence encodes the first and/or second coat protein(s) of the bacteriophage.

168. The system according to claim 167, wherein the first nucleic acid sequence is a wild-type genome of the bacteriophage.

169. The system according to any one of claims 163 to 168, wherein at least one of the first and second coat proteins is a nonessential outer capsid protein of the bacteriophage.

170. The system according to any one of claims 163 to 168, wherein the bacteriophage is a non-naturally occurring T4 phage, T7 phage, or λ (lambda) phage.

171. The system according to any one of claims 163 to 170, wherein the first nucleic acid sequence and the second nucleic acid sequence are in separate nucleic acid molecules.

172. The system according to claim 171, further comprising a site-specific recombinase capable of catalyzing homologous recombination between the first and second nucleic acid sequences to produce a recombinant sequence; wherein the recombinant sequence encodes for the one or more structural proteins of the bacteriophage and the first and/or second fusion protein.

173. The system according to claim 171 or 172, wherein the mutated phage genome is T4 phage genome devoid of one or more sequence coding for the first and/or second nonessential outer capsid protein(s).

174. The system according to any one of claims 171 to 173, wherein the second nucleic acid sequence is a plasmid.

175. The system according to any one of claims 163 to 170, wherein the first nucleic acid sequence and the second nucleic acid sequence are in the same nucleic acid molecule.

176. The system according to claim 175, wherein the nucleic acid molecule is a mutated genome of the bacteriophage devoid of one or more endogenous sequence encoding the first and/or second nonessential outer capsid protein(s).

177. The system according to claim 176, wherein the second sequence is an exogenous sequence.

178. The system according to any one of claims 175 to 177, wherein the nucleic acid molecule is a mutated genome of the T4 phage with endogenous sequences coding for HOC and/or SOC deleted.

179. The system according to claim 178, wherein the second sequence encodes a fusion protein comprising the lasso peptide component fused to HOC.

180. The system according to claim 179, wherein the second sequence encodes a fusion protein comprising the identification peptide fused to SOC.

181. The system according to any one of claims 163 to 180, wherein the lasso peptide component is a lasso precursor peptide, a lasso core peptide, a lasso peptide or a functional fragment of lasso peptide.

182. The system according to claim 181, wherein the lasso precursor peptide comprises a sequence of any one of the even numbers of SEQ ID NOS:1-2630, or a sequence having greater than 30% identity of any one of the even numbers of SEQ ID NOS:1-2630.

183. The system according to any one of claims 163 to 182, wherein the nucleic acid comprises (i) a sequence of any one of the odd numbers of SEQ ID NOS:1-2630, (ii) a sequence having greater than 30% identity of any one of the odd numbers of SEQ ID NOS:1-2630, or (iii) a sequence encoding a polypeptide having greater than 30% identity of any one of the even numbers of SEQ ID NOS:1-2630.

184. The system according to any one of claims 163 to 183, wherein the third nucleic acid sequence encodes one or more lasso peptide biosynthesis component.

185. The system according to claim 184, wherein the at least one lasso peptide biosynthesis component comprises one or more of a lasso peptidase, a lasso cyclase and a lasso RRE.

186. The system according to claim 185, wherein the third nucleic acid sequence encodes a lasso peptidase.

187. The system according to claim 186, wherein the third nucleic acid sequence further encodes a lasso cyclase.

188. The system according to claim 187, wherein the third nucleic acid sequence further encodes a lasso RRE.

189. The system according to claim 185, wherein the third nucleic acid sequence encodes a fusion protein comprising a lasso peptidase and a lasso cyclase.

190. The system according to claim 189, wherein the third nucleic acid sequence further encodes a lasso RRE.

191. The system according to claim 185, wherein the third nucleic acid sequence encodes a fusion protein comprising a lasso peptidase and a lasso RRE.

192. The system according to claim 190, wherein the third nucleic acid sequence further encodes a lasso cyclase.

193. The system according to claim 185, wherein the third nucleic acid sequence encodes a fusion protein comprising a lasso cyclase and a lasso RRE.

194. The system according to claim 193, wherein the third nucleic acid sequence further encodes a lasso peptidase.

195. The system according to claim 185, wherein the third nucleic acid sequence encodes a fusion protein comprising a lasso peptidase, a lasso cyclase, and a lasso RRE.

196. The system according to any one of claims 163 to 195, wherein the third nucleic acid sequence comprises a sequence encoding a polypeptide having greater than 30% identify of any one of peptide Nos: 1316-2336, peptide Nos: 2337-3761, and peptide Nos: 3762-4593.

197. The system according to any one of claims 163 to 196, wherein the third nucleic acid sequence is one or more plasmid.

198. The system according to any one of claims 163 to 197, further comprising a microbial cell having cytoplasm, wherein the first, second and third nucleic acid sequences are in the cytoplasm of the microbial cell.

199. The system according to any one of claims 163 to 198, wherein the microbial cell is a bacterial cell or an archaea cell.

200. The system according to claim 199, wherein the bacterial cell is E. coli.

201. The system according to any one of claims 163 to 200, further comprising a cell-free biosynthesis reaction mixture, wherein the first, second and third nucleic acid sequence are in the cell-free biosynthesis reaction mixture.

202. The system according to any one of claim 163 to 201, wherein the identification peptide is a purification tag.

203. The nucleic acid molecule according to claim 202, wherein the purification tag is Albumin-binding protein (ABP), Alkaline Phosphatase (AP), AU1 epitope, AU5 epitope, Bacteriophage T7 epitope (T7 tag), Bacteriophage V5 epitope (V5 tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1-tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

204. The system according to any one of claim 163 to 203, wherein the first fusion protein further comprises a linker between the first protein and the lasso peptide component.

205. The system according to 204, wherein the liner is a cleavable linker.

206. A system comprising a bacteriophage devoid of a first nonessential outer capsid protein, and a first fusion protein comprising a lasso peptide component fused to the first nonessential outer capsid protein of the bacteriophage.

207. The system according to claim 206, wherein the bacteriophage is devoid of a second nonessential outer capsid protein, and wherein the system further comprises a second fusion protein comprising an identification peptide fused to the second nonessential outer capsid protein of the bacteriophage.

208. The system according to claim 206 or 207, wherein the bacteriophage comprises a mutated genome having one or more endogenous sequence encoding the first and/or second nonessential outer capsid protein(s) of the bacteriophage deleted.

209. The system according to claim 208, wherein the mutated genome further comprising an exogenous sequence encoding the first and/or second fusion protein.

210. The system according to any one of claims 206 to 209, wherein the bacteriophage is a non-naturally occurring T4 phage, T7 phage or λ (lambda) phage.

211. The system according to any one of claims 206 to 210, wherein the bacteriophage is a non-naturally occurring T4 phage, and wherein the first nonessential outer capsid protein is HOC and the second nonessential outer capsid protein is SOC.

212. The system according to any one of claims 206 to 211, wherein the lasso peptide component is a lasso precursor peptide, a lasso core peptide, a lasso peptide or a functional fragment of lasso peptide.

213. The system according to claim 212, further comprises at least one lasso peptide biosynthesis component.

214. The system according to any one of claims 206 to 213, wherein the bacteriophage, the first and/or second fusion protein(s), and/or the at least one lasso peptide biosynthesis component is in a cytoplasm of the host microbial cell.

215. The system according to any one of claims 206 to 213, wherein the bacteriophage, the first and/or second fusion protein(s), and/or the at least one lasso peptide biosynthesis component is in a cell-free biosynthesis reaction mixture.

216. The system according to any one of claims 206 to 213, wherein the bacteriophage, the first and/or second fusion protein(s), and/or the at least one lasso peptide biosynthesis component is purified.

217. The system according to any one of claims 206 to 216 further comprising a solid support having at least one unique location, wherein the bacteriophage, the first and/or second fusion protein(s), and/or the at least one lasso peptide biosynthesis component is located at the unique location.

218. The system according to claim 217, wherein the lasso precursor peptide comprises a sequence of any one of the even numbers of SEQ ID NOS:1-2630, or a sequence having greater than 30% identity of any one of the even numbers of SEQ ID NOS:1-2630.

219. The system according to any one of claims 213 to 218, wherein the at least one lasso peptide biosynthesis component comprises one or more of a lasso peptidase, a lasso cyclase and a lasso RRE.

220. The system according to claim 219, wherein the lasso peptidase comprises a sequence of any one of peptide Nos: 1316-2336, or a sequence having greater than 30% identity of any one of peptide Nos: 1316-2336.

221. The system according to claim 219, wherein the lasso cyclase comprises a sequence of any one of peptide Nos: 2337-3761, or a sequence having greater than 30% identity of any one of peptide Nos: 2337-3761.

222. The system according to claim 219, wherein the lasso RRE comprises a sequence of any one of peptide Nos: 3762-4593, or a sequence having greater than 30% identity of any one of peptide Nos: 3762-4593.

223. The system according to any one of claims 213 to 218, wherein the at least one lasso peptide biosynthesis component comprises a fusion protein comprising a lasso peptidase and a lasso cyclase.

224. The system according to any one of claims 213 to 218, wherein the at least one lasso peptide biosynthesis component comprises a fusion protein comprising a lasso peptidase and a lasso RRE.

225. The system according to claim 224, wherein the fusion protein comprising the lasso peptidase and the lasso RRE comprises a sequence of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562, or a sequence having greater than 30% identity of any one of peptide Nos: 3768, 3770, 3793, 3811, 3818, 3851, 3855, 3887, 4004, 4018, 4045, 4076, 4132, 4150, 4167, 4168, 4225, 4262, 4379, 4414, 4499, 4504, 4507, 4512, 4517, 4518, 4529, 4532, 4542, 4559, 4561, 4562.

226. The system according to any one of claims 213 to 218, wherein the at least one lasso peptide biosynthesis component comprises a fusion protein comprising a lasso cyclase and a lasso RRE.

227. The system according to claim 226, wherein the fusion protein comprising the lasso cyclase and the lasso RRE comprises a sequence selected from peptide Nos: 2504, 3608 or a sequence having greater than 30% identity of any one of peptide Nos: 2504 and 3608.

228. The system according to any one of claims 213 to 218, wherein the at least one lasso peptide biosynthesis component comprises a fusion protein comprising a lasso peptidase and a lasso cyclase.

229. The system according to claim 228, wherein the fusion protein comprising the lasso peptidase and the lasso cyclase comprises a sequence having peptide No: 2903 or a sequence having greater than 30% identity thereof.

230. The system according to any one of claims 213 to 218, wherein the at least one lasso peptide biosynthesis component comprises a fusion protein comprising a lasso peptidase, a lasso cyclase, and a lasso RRE.

231. The system according to claim 214, wherein the host microbial cell is a bacterial cell or an archaeal cell.

232. The system according to claim 231, wherein the host microbial cell is E. coli.

233. The system according to any one of claims 207 to 232, wherein the identification peptide is a purification tag.

234. The system according to any one of claims 206 to 233, wherein the system further comprises a solid support having at least one unique location.

235. The system according to claim 233, wherein the purification tag is Albumin-binding protein (ABP), Alkaline Phosphatase (AP), AU1 epitope, AU5 epitope, Bacteriophage 17 epitope (T7 tag), Bacteriophage V5 epitope (V5-tag), Biotin-carboxy carrier protein (BCCP), Bluetongue virus tag (B tag), Calmodulin binding peptide (CBP), Chloramphenicol Acetyl Transferase (CAT), Cellulose binding domain (CBD), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione-S transferase (GST), Human influenza hemagglutinin (HA), HaloTag®, Histidine affinity tag (HAT), Horseradish peroxidase (HRP), HSV epitope, Ketosteroid isomerase (KSI), KT3 epitope, LacZ, Luciferase, Maltose-binding protein (MBP), Myc epitope, NusA, PDZ ligand, Polyarginine (Arg-tag), Polyaspartate (Asp-tag), Polycysteine (Cys-tag), Polyhistidine (His-tag), Polyphenylalanine (Poly-tag), Profinity eXact™, Protein C, S1-tag, S-tag, Streptavidin-binding peptide (SBP), Staphylococcal protein A (Protein A), Staphylococcal protein G (Protein G), Strep-tag, Streptavidin, Small Ubiquitin-like Modifier (SUMO), Tandem Affinity Purification (TAP), T7 epitope, Thioredoxin (Trx), TrpE, Ubiquitin, Universal, VSV-G.

236. The system according to any one of claim 206 to 235, wherein the first fusion protein further comprises a linker between the first protein and the lasso peptide component.

237. The system according to 236, wherein the liner is a cleavable linker.

238. A bacteriophage comprising a genome and a capsid, wherein the capsid comprises a plurality of a first coat proteins, and wherein at least one of the first coat proteins is fused to a lasso peptide component in a first fusion protein.

239. The bacteriophage according to claim 238, further comprises a plurality of a second coat protein, and wherein at least one of the second coat protein is fused to an identification peptide in a second fusion protein.

240. The bacteriophage according to claim 238 or 239, wherein the genome is devoid of one or more endogenous sequence encoding the first and/or second coat protein(s).

241. The bacteriophage according to claim 240, wherein the genome further comprises an exogenous sequence encoding the first and/or second fusion protein.

242. The bacteriophage according to claim 236 or 239, wherein the genome is a wild-type genome.

243. The bacteriophage according to any one of claims 238 to 242, wherein at least one first coat protein is wild-type.

244. The bacteriophage according to any one of claims 238 to 243, wherein at least one second coat protein is wild-type.

245. The bacteriophage according to claim 238, wherein the genome is wild-type, and wherein the capsid comprises at least one first coat protein in the first fusion protein, and at least one first coat protein that is wild-type.

246. The bacteriophage according to claim 245, wherein the capsid further comprises at least one second coat protein in the second fusion protein, and at least one second coat protein that is wild-type.

247. The bacteriophage according to claim 238, wherein the genome is devoid of an endogenous sequence coding for the first coat protein, and wherein the capsid comprises at least one first coat protein in the first fusion protein.

248. The bacteriophage according to claim 247, wherein the genome further comprises an exogenous sequence encoding the first fusion protein.

249. The bacteriophage according to claim 248, wherein the capsid further comprises at least one first coat protein that is wild-type.

250. The bacteriophage according to any one of claims 247 to 249, wherein the genome is further devoid of an endogenous sequence coding for the second coat protein, and wherein the capsid comprises at least one second coat protein in the second fusion protein.

251. The bacteriophage according to claim 250, wherein the capsid further comprises at least one second coat protein that is wild-type.

252. The bacteriophage according to any one of claims 238 to 251, wherein the first coat protein is a nonessential outer capsid protein.

253. The bacteriophage according to claim 252, wherein the second coat protein is a nonessential outer capsid protein.

254. The bacteriophage according to any one of claims 238 to 253, wherein the bacteriophage is a non-naturally occurring T4 phage, T7 phage or a λ (lambda) phage.

255. The bacteriophage according to any one of claims 238 to 254, wherein the bacteriophage is a non-naturally occurring T4 phage, and wherein the first coat protein is HOC and the second coat protein is SOC.

256. The bacteriophage according to any one of claims 238 to 255, wherein the bacteriophage is capable of infection of a host microbial cell.

257. The bacteriophage according to any one of claims 238 to 256, herein the host microbial organism is a bacterial cell or an archaea cell.

258. The bacteriophage according to any one of claims 238 to 257, wherein the host microbial organism is E. coli.

259. The bacteriophage according to any one of claims 238 to 258, wherein the lasso peptide component is a lasso precursor peptide, a lasso core peptide, a lasso peptide or a functional fragment of lasso peptide.

260. The bacteriophage according to claim 259, wherein the lasso precursor peptide comprises a sequence of any one of the even numbers of SEQ ID NOS:1-2630, or a sequence having greater than 30% identity of any one of the even numbers of SEQ ID NOS:1-2630.

261. A library comprising a plurality of distinct members, wherein each member is bacteriophage according any one of claims 238 to 260, wherein the first fusion proteins in the distinct members comprise distinct lasso peptide components.

262. The library according to claim 261, further comprising a solid support comprising a plurality of unique locations, wherein each unique location contains a distinct member.

263. A method for making a member of a bacteriophage display library comprising
providing a system comprising (i) a first nucleic acid sequence encoding one or more structural proteins of a bacteriophage; (ii) a second nucleic acid sequence encoding a first fusion protein comprising a lasso peptide component fused to a first coat protein of the bacteriophage; and (iii) a third nucleic acid sequence encoding at least one lasso peptide biosynthesis component.
introducing the system into a population of microbial cells or a cell-free biosynthesis reaction mixture;
incubating the population of microbial cells or the cell-free biosynthesis reaction mixture under a suitable condition to produce a plurality of bacteriophages each displaying the lasso peptide component on the first coat protein; and
wherein the lasso peptide biosynthesis component processes the lasso peptide component into a lasso peptide or a functional fragment of lasso peptide.

264. The method of claim 263, wherein the first nucleic acid sequence comprises a mutated genome of the bacteriophage devoid of an endogenous sequence encoding the first coat protein.

265. The method of claim 264, wherein the first nucleic acid sequence and the second nucleic acid sequence are in the same nucleic acid molecule.

266. The method of claim 264, wherein the first, second and third nucleic acid sequences are in the same nucleic acid molecule.

267. The method of claim 264, wherein the first nucleic acid sequence and the second nucleic acid sequence in different nucleic acid molecules that are configured to undergo homologous recombination to produce a recombinant sequence encoding the structural proteins and the first fusion protein.

268. The method of any one of claim 263 to 267, wherein the step of introducing the system into the population of microbial cells comprises infecting the population of microbial cells with a bacteriophage having a mutated genome comprising the first nucleic acid.

269. The method of any one of claim 263 to 268, wherein the step of introducing the system into the population of microbial cells comprises transfecting the population of microbial cells with one or more vectors comprising the second and/or third nucleic acid sequence.

270. The method of any one of claims 264 to 269
wherein the first nucleic acid comprises a mutated genome of the bacteriophage devoid of an endogenous sequence encoding a second coat protein of the bacteriophage,
wherein the second nucleic acid sequence further encodes a second fusion protein comprising an identification peptide fused to the second coat protein; and
wherein the step of incubating comprises incubating the population of microbial cells or cell-free biosynthesis reaction mixture under a suitable condition to produce a plurality of bacteriophages each displaying the lasso peptide component on the first coat protein and the identification peptide on the second coat protein.

271. The method of claim 270, further comprising identifying the lasso peptide component based on the identification peptide.

272. The method of claim 271, wherein the identification peptide is a purification tag, and the method further comprises purifying the produced plurality of bacteriophages.

273. The method of claim 263, wherein the first nucleic acid sequence comprises a wild-type genome of the bacteriophage.

274. The method of claim 263, wherein the one or more structural proteins encoded by the first nucleic acid sequence comprises wild-type first coat protein.

275. The method of claim 274, wherein the first and second nucleic acid sequences are in the same nucleic acid molecule.

276. The method of claim 274,
wherein the one or more structural proteins encoded by the first nucleic acid sequence further comprises a wild-type second coat protein;
wherein the second nucleic acid sequence further encodes a second fusion protein comprising an identification peptide fused to the second coat protein; and
wherein the step of incubating comprises incubating the population of microbial cells or cell-flee biosynthesis reaction mixture under a suitable condition to produce a plurality of bacteriophages each comprising the wild-type second coat protein and the second fusion protein.

277. The method of claim 276, further comprising identifying the lasso peptide component based on the identification peptide.

278. The method of claim 276, wherein the identification peptide is a purification tag, and the method further comprises purifying the produced plurality of bacteriophages.

279. The method of any one of claims 275 to 276, wherein the first, second and third nucleic acid sequences are in the same nucleic acid molecule.

280. The method of any one of claim 275 to 279, wherein the nucleic acid molecule comprises a mutated genome of the bacteriophage.

281. The method of any one of claims 263 to 280, wherein the step of incubating is performed at a unique location configured to identify the lasso peptide component.

282. The method of claim 281, further comprising identifying the lasso peptide component based on the unique location.

283. The method of any one of claims 263 to 282, wherein the bacteriophage is a non-naturally occurring T4 page, T7 phage or λ (lambda) phage.

284. The method of any one of claims 263 to 283, wherein the bacteriophage is a non-naturally occurring T4 page, and wherein the first coat protein is HOC and the second coat protein is SOC.

285. A method for making a member of a bacteriophage display library comprising contacting a first bacteriophage devoid of a first nonessential outer capsid protein with a first fusion protein comprising a lasso peptide component fused to the first nonessential outer capsid protein of the bacteriophage under a suitable condition to produce a second bacteriophage displaying the lasso peptide component on the first coat protein.

286. The method of claim 285,
wherein the first bacteriophage is further devoid of a second nonessential outer capsid protein, and
wherein the method further comprises contacting the second bacteriophage with a second fusion protein comprising an identification peptide fused with the second nonessential outer capsid protein under a suitable condition to produce a third bacteriophage displaying the lasso peptide component on the first coat protein and the identification peptide on the second coat protein.

287. The method of claim 285 or 286, further comprising contacting the second or the third bacteriophage with at least one lasso peptide biosynthesis component under a suitable condition to process the lasso peptide component into a lasso peptide or a functional fragment of lasso peptide.

288. The method of any one of claims 285 to 287, wherein the first bacteriophage comprises a mutated genome devoid of an endogenous sequence encoding the first nonessential outer capsid protein.

289. The method of any one of claims 285 to 288, wherein the first bacteriophage comprises a mutated genome devoid of an endogenous sequence encoding the second nonessential outer capsid protein.

290. The method of any one of claims 285 to 289, wherein the first bacteriophage comprises a mutated genome comprising an exogenous sequence encoding the first fusion protein.

291. The method of any one of claims 285 to 290, wherein the first bacteriophage comprises a mutated genome comprising an exogenous sequence encoding the second fusion protein.

292. The method of any one of claims 285 to 287, wherein the first bacteriophage comprises a wild-type genome of the bacteriophage.

293. The method of any one of claims 285 to 292, wherein the second or third bacteriophage is a non-naturally existing T4 phage, T7 phage or λ (lambda) phage.

294. The method of any one of claims 285 to 293, wherein the second or third bacteriophage is a non-naturally existing T4 phage, and wherein the first nonessential outer capsid protein is HOC, and the second nonessential outer capsid protein is SOC.
